(19) United States
(12) Patent Application Publication
(10) Pub. No.: US 2004/0142331 A1
(43) Pub. Date: Jul. 22, 2004

(54) Molecules for disease detection and treatment

(76) Inventors: Stuart E Jackson, Santa Cruz, CA (US); Stephen E Lincoln, Potomac, MD (US); Christina M Altus, Campbell, CA (US); Gerard E Dufour, Castro Valley, CA (US); Michael S Chalup, Livingston, TX (US); Jennier L Jackson, Santa Cruz, CA (US); Anissa L Jones, San Jose, CA (US); Jimmy Y Yu, Fremont, CA (US); Rachel J Wright, Mountain View, CA (US); Darryl Gietzen, San Jose, CA (US); Tommy F Liu, Daly City, CA (US); Pierre E Yap, Lafayette, CA (US); Christopher R Dahl, Longview, TX (US); Monika G Momiyama, Hayward, CA (US); Diana L Bradley, Soquel, CA (US); Sameer D Rohatgi, San Francisco, CA (US); Bernard Harris, Sunnyvale, CA (US); Ann M Roseberry Lincoln, Potomac, MD (US); Edward H Gerstin JR., San Jose, CA (US); Careyna H Peralta, Santa Clara, CA (US); Marie H David, Daly City, CA (US); Scott R Panzer, Sunnyvale, CA (US); Vincent Z Flores, Union City, CA (US); Abel Daffo, San Jose, CA (US); Rakesh Marwaha, Burnaby (CA); Alice J Chen, San Jose, CA (US); Simon C Chang, Sunnyvale, CA (US); Alan P Au, Mountain View, CA (US); Rebekah R Inman, Mountain View, CA (US)

Correspondence Address: INCYTE CORPORATION, 3160 PORTER DRIVE, PALO ALTO, CA, 94304, US

(21) Appl. No.: 10/363,829

(22) PCT Filed: Sep. 5, 2001
(86) PCT No.: PCT/US01/27628

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention provides purified disease detection and treatment molecule polynucleotides (mddt). Also encompassed are the polypeptides (MDDT) encoded by mddt. The invention also provides for the use of mddt, or complements, oligonucleotides, or fragments thereof in diagnostic assays. The invention further provides for vectors and host cells containing mddt for the expression of MDDT. The invention additionally provides for the use of isolated and purified MDDT to induce antibodies and to screen libraries of compounds and the use of anti-MDDT antibodies in diagnostic assays. Also provided are microarrays containing mddt and methods of use.

### TECHNICAL FIELD

The present invention relates to molecules for disease detection and treatment and to the use of these sequences in the diagnosis, study, prevention, and treatment of diseases associated with, as well as effects of exogenous compounds on, the expression of molecules for disease detection and treatment.

### BACKGROUND OF THE INVENTION

The human genome is comprised of thousands of genes, many encoding gene products that function in the maintenance and growth of the various cells and tissues in the body. Aberrant expression or mutations in these genes and their products is the cause of, or is associated with, a variety of human diseases such as cancer and other cell proliferative disorders. The identification of these genes and their products is the basis of an ever-expanding effort to find markers for early detection of diseases, and targets for their prevention and treatment.

For example, cancer represents a type of cell proliferative disorder that affects nearly every tissue in the body. A wide variety of molecules, either aberrantly expressed or mutated, can be the cause of, or involved with, various cancers because tissue growth involves complex and ordered patterns of cell proliferation, cell differentiation, and apoptosis. Cell proliferation must be regulated to maintain both the number of cells and their spatial organization. This regulation depends upon the appropriate expression of proteins which control cell cycle progression in response to extracellular signals such as growth factors and other mitogens, and intracellular cues such as DNA damage or nutrient starvation. Molecules which directly or indirectly modulate cell cycle progression fall into several categories, including growth factors and their receptors, second messenger and signal transduction proteins, oncogene products, tumor-suppressor proteins, and mitosis-promoting factors. Aberrant expression or mutations in any of these gene products can result in cell proliferative disorders such as cancer. Oncogenes are genes generally derived from normal genes that, through abnormal expression or mutation, can effect the transformation of a normal cell to a malignant one (oncogenesis). Oncoproteins, encoded by oncogenes, can affect cell proliferation in a variety of ways and include growth factors, growth factor receptors, intracellular signal transducers, nuclear transcription factors, and cell-cycle control proteins. In contrast, tumor-suppressor genes are involved in inhibiting cell proliferation. Mutations which cause reduced or loss of function in tumor-suppressor genes result in aberrant cell proliferation and cancer. Thus a wide variety of genes and their products have been found that are associated with cell proliferative disorders such as cancer, but many more may exist that are yet to be discovered.

DNA-based arrays can provide a simple way to explore the expression of a single polymorphic gene or a large number of genes. When the expression of a single gene is explored, DNA-based arrays are employed to detect the expression of specific gene variants. For example, a p53 tumor suppressor gene array is used to determine whether individuals are carrying mutations that predispose them to cancer. A cytochrome p450 gene array is useful to determine whether individuals have one of a number of specific mutations that could result in increased drug metabolism, drug resistance or drug toxicity.

DNA-based array technology is especially relevant for the rapid screening of expression of a large number of genes. There is a growing awareness that gene expression is affected in a global fashion. A genetic predisposition, disease or therapeutic treatment may affect, directly or indirectly, the expression of a large number of genes. In some cases the interactions may be expected, such as when the genes are part of the same signaling pathway. In other cases, such as when the genes participate in separate signaling pathways, the interactions may be totally unexpected. Therefore, DNA-based arrays can be used to investigate how genetic predisposition, disease, or therapeutic treatment affects the expression of a large number of genes.

The discovery of new molecules for disease detection and treatment satisfies a need in the art by providing new compositions which are useful in the diagnosis, study, prevention, and treatment of diseases associated with, as well as effects of exogenous compounds on, the expression of molecules for disease detection and treatment.

### SUMMARY OF THE INVENTION

The present invention relates to human disease detection and treatment molecule polynucleotides (mddt) as presented in the Sequence Listing. The mddt uniquely identify genes encoding structural, functional, and regulatory disease detection and treatment molecules.

The invention provides an isolated polynucleotide selected from the group consisting of a) a polynucleotide comprising a polynucleotide sequence selected from the group consisting of SEQ ID NO:1-252; b) a polynucleotide comprising a naturally occurring polynucleotide sequence at least 90% identical to a polynucleotide sequence selected from the group consisting of SEQ ID NO:1-252; c) a polynucleotide complementary to the polynucleotide of a); d) a polynucleotide complementary to the polynucleotide of b); and e) an RNA equivalent of a) through d). In one alternative, the polynucleotide comprises a polynucleotide sequence selected from the group consisting of SEQ ID NO:1-252. In another alternative, the polynucleotide comprises at least 30 contiguous nucleotides of a polynucleotide selected from the group consisting of a) a polynucleotide comprising a polynucleotide sequence selected from the group consisting of SEQ ID NO:1-252; b) a polynucleotide comprising a naturally occurring polynucleotide comprising a polynucleotide sequence at lease identical to a polynucleotide sequence selected from the group consisting of SEQ ID NO:1-252; c) a polynucleotide complementary to the polynucleotide of a); d) a polynucleotide complementary to the polynucleotide of b); and e) an RNA equivalent of a) through d). In another alternative, the polynucleotide comprises at least 60 contiguous nucleotides of a polynucleotide selected from the group consisting of a) a polynucleotide comprising a polynucleotide sequence selected from the group consisting of SEQ ID NO:1-252; b) a polynucleotide comprising a naturally occurring polynucleotide comprising a polynucleotide sequence at least 90% identical to a polynucleotide sequence selected from the group consisting of SEQ ID NO:1-252; c) a polynucleotide complementary to the polynucleotide of a); d) a polynucleotide complementary to the polynucleotide of b); and e) an RNA equivalent of a) through d). The invention further provides a composition for the detection of expression of disease detection and treatment molecule polynucleotides comprising at least one isolated polynucleotide comprising a polynucleotide selected from the group consisting of a) a polynucleotide comprising a polynucleotide sequence selected from the group consisting of SEQ ID NO:1-252; b) a polynucleotide comprising a naturally occurring polynucleotide sequence at least 90% identical to a polynucleotide sequence selected from the group consisting of SEQ ID NO:1-252; c) a polynucleotide complementary to the polynucleotide of a); d) a polynucleotide complementary to the polynucleotide of b); and e) an RNA equivalent of a) through d); and a detectable label.

The invention also provides a method for detecting a target polynucleotide in a sample, said target polynucleotide having a polynucleotide sequence of a polynucleotide selected from the group consisting of a) a polynucleotide comprising a polynucleotide sequence of a polynucleotide selected from the group consisting of SEQ ID NO:1-252; b) a polynucleotide comprising a naturally occurring polynucleotide sequence at least 90% identical to a polynucleotide sequence selected from the group consisting of SEQ ID NO:1-252; c) a polynucleotide complementary to the polynucleotide of a); d) a polynucleotide complementary to the polynucleotide of b); and e) an RNA equivalent of a) through d). The method comprises a) amplifying said target polynucleotide or fragment thereof using polymerase chain reaction amplification, and b) detecting the presence or absence of said amplified target polynucleotide or fragment thereof, and, optionally, if present, the amount thereof.

The invention also provides a method for detecting a target polynucleotide in a sample, said target polynucleotide having a polynucleotide selected from the group consisting of a) a polynucleotide comprising a polynucleotide sequence selected from the group consisting of SEQ ID NO:1-252; b) a polynucleotide comprising a naturally occurring polynucleotide sequence at least 90% identical to a polynucleotide sequence selected from the group consisting of SEQ ID NO:1-252; c) a polynucleotide complementary to the polynucleotide of a); d) a polynucleotide complementary to the polynucleotide of b); and e) an RNA equivalent of a) through d). The method comprises a) hybridizing the sample with a probe comprising at least 60 contiguous nucleotides comprising a sequence complementary to said target polynucleotide in sample, and which probe specifically hybridizes to said target polynucleotide, under conditions whereby a hybridization complex is formed between said probe and said target polynucleotide, and b) detecting the presence or absence of said hybridization complex, and, optionally, if present, the amount thereof. In one alternative, the invention provides a composition comprising a target polynucleotide of the method, wherein said probe comprises at least 30 contiguous nucleotides. In one alternative, the invention provides a composition comprising a target polynucleotide of the method, wherein said probe comprises at least 60 contiguous nucleotides.

The invention further provides a recombinant polynucleotide comprising a promoter sequence operably linked to an isolated polynucleotide selected from the group consisting of a) a polynucleotide comprising a polynucleotide sequence selected from the group consisting of SEQ ID NO:1-252; b) a polynucleotide comprising a naturally occurring polynucleotide sequence at least 90% identical to a polynucleotide sequence selected from the group consisting of SEQ ID NO:1-252; c) a polynucleotide complementary to the polynucleotide of a); d) a polynucleotide complementary to the polynucleotide of b); and e) an RNA equivalent of a) through d). In one alternative, the invention provides a cell transformed with the recombinant polynucleotide. In another alternative, the invention provides a transgenic organism comprising the recombinant polynucleotide.

The invention also provides a method for producing a disease detection and treatment polypeptide, the method comprising a) culturing a cell under conditions suitable for expression of the disease detection and treatment polypeptide, wherein said cell is transformed with a recombinant polynucleotide, said recombinant polynucleotide comprising an isolated polynucleotide selected from the group consisting of i) a polynucleotide comprising a polynucleotide sequence selected from the group consisting of SEQ ID NO:1-252; ii) a polynucleotide comprising a naturally occurring polynucleotide sequence at least 90% identical to a polynucleotide sequence selected from the group consisting of SEQ ID NO:1-252; iii) a polynucleotide complementary to the polynucleotide of i); iv) a polynucleotide complementary to the polynucleotide of ii); and v) an RNA equivalent of i) through iv), and b) recovering the disease detection and treatment polypeptide so expressed. The invention additionally provides a method wherein the polypeptide has an amino acid sequence selected from the group consisting of SEQ ID NO:253-506.

The invention also provides an isolated disease detection and treatment polypeptide (MDDT) encoded by at least one polynucleotide comprising a polynucleotide sequence selected from the group consisting of SEQ ID NO:1-252. The invention further provides a method of screening for a test compound that specifically binds to the polypeptide having an amino acid sequence selected from the group consisting of SEQ ID NO:253-506. The method comprises a) combining the polypeptide having an amino acid sequence selected from the group consisting of SEQ ID NO:253-506 with at least one test compound under suitable conditions, and b) detecting binding of the polypeptide having an amino acid sequence selected from the group consisting of SEQ ID NO:253-506 to the test compound, thereby identifying a compound that specifically binds to the polypeptide having an amino acid sequence selected from the group consisting of SEQ ID NO:253-506.

The invention further provides a microarray wherein at least one element of the microarray is an isolated polynucleotide comprising at least 30 contiguous nucleotides of a polynucleotide selected from the group consisting of a) a polynucleotide comprising a polynucleotide sequence selected from the group consisting of SEQ ID NO:1-252; b) a polynucleotide comprising a naturally occurring polynucleotide sequence at least 90% identical to a polynucleotide sequence selected from the group consisting of SEQ ID NO:1-252; c) a polynucleotide complementary to the polynucleotide of a); d) a polynucleotide complementary to the polynucleotide of b); and e) an RNA equivalent of a) through d). The invention also provides a method for generating a transcript image of a sample which contains polynucleotides. The method comprises a) labeling the polynucleotides of the sample, b) contacting the elements of the microarray with the labeled polynucleotides of the sample under conditions suitable for the formation of a hybridization complex, and c) quantifying the expression of the polynucleotides in the sample.

Additionally, the invention provides a method for screening a compound for effectiveness in altering expression of a target polynucleotide, wherein said target polynucleotide comprises a polynucleotide selected from the group consisting of a) a polynucleotide comprising a polynucleotide sequence selected from the group consisting of SEQ ID NO:1-252; b) a polynucleotide comprising a naturally occurring polynucleotide sequence at least 90% identical to a polynucleotide sequence selected from the group consisting of SEQ ID NO:1-252; c) a polynucleotide complementary to the polynucleotide of a); d) a polynucleotide complementary to the polynucleotide of b); and e) an RNA equivalent of a) through d). The method comprises a) exposing a sample comprising the target polynucleotide to a compound, b) detecting altered expression of the target polynucleotide, and c) comparing the expression of the target polynucleotide in the presence of varying amounts of the compound and in the absence of the compound.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:1-252; ii) a polynucleotide comprising a naturally occurring polynucleotide sequence at least 90% identical to a polynucleotide sequence selected from the group consisting of SEQ ID NO:1-252; iii) a polynucleotide complementary to the polynucleotide of i); iv) a polynucleotide complementary to the polynucleotide of ii); and v) an RNA equivalent of i) through iv). Hybridization occurs under conditions whereby a specific hybridization complex is formed between said probe and a target polynucleotide in the biological sample, said target polynucleotide comprising a polynucleotide sequence of a polynucleotide selected from the group consisting of i) a polynucleotide comprising a polynucleotide sequence selected from the group consisting of SEQ ID NO:11-252; ii) a polynucleotide comprising a naturally occurring polynucleotide sequence at least 90% identical to a polynucleotide sequence selected from the group consisting of SEQ ID NO:11-252; iii) a polynucleotide complementary to the polynucleotide of i); iv) a polynucleotide complementary to the polynucleotide of ii); and v) an RNA equivalent of i) through iv), and alternatively, the target polynucleotide comprises a polynucleotide sequence of a fragment of a polynucleotide selected from the group consisting of i-v above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

The invention further provides an isolated polypeptide selected from the group consisting of a) a polypeptide comprising an amino acid sequence selected from the group consisting of SEQ ID NO:253-506, b) a polypeptide comprising a naturally occurring amino acid sequence at least 90% identical to an amino acid sequence selected from the group consisting of SEQ ID NO:253-506, c) a biologically active fragment of a polypeptide having an amino acid sequence selected from the group consisting of SEQ ID NO:253-506, and d) an immunogenic fragment of a polypeptide having an amino acid sequence selected from the group consisting of SEQ ID NO:253-506. In one alternative, the invention provides an isolated polypeptide comprising an amino acid sequence selected from the group consisting of SEQ ID NO:253-506.

The invention further provides an isolated polynucleotide encoding a polypeptide selected from the group consisting of a) a polypeptide comprising an amino acid sequence selected from the group consisting of SEQ ID NO:253-506, b) a polypeptide comprising a naturally occurring amino acid sequence at least 90% identical to an amino acid sequence selected from the group consisting of SEQ ID NO:253-506, c) a biologically active fragment of a polypeptide having an amino acid sequence selected from the group consisting of SEQ ID NO:253-506, and d) an immunogenic fragment of a polypeptide having an amino acid sequence selected from the group consisting of SEQ ID NO:253-506. In one alternative, the polynucleotide encodes a polypeptide comprising an amino acid sequence selected from the group consisting of SEQ ID NO:253-506. In another alternative, the polynucleotide comprises a polynucleotide sequence selected from the group consisting of SEQ ID NO:1-252.

Additionally, the invention provides an isolated antibody which specifically binds to a polypeptide selected from the group consisting of a) a polypeptide comprising an amino acid sequence selected from the group consisting of SEQ ID NO:253-506, b) a polypeptide comprising a naturally occurring amino acid sequence at least 90% identical to an amino acid sequence selected from the group consisting of SEQ ID NO:253-506, c) a biologically active fragment of a polypeptide having an amino acid sequence selected from the group consisting of SEQ ID NO:253-506, and d) an immunogenic fragment of a polypeptide having an amino acid sequence selected from the group consisting of SEQ ID NO:253-506.

The invention further provides a composition comprising a polypeptide selected from the group consisting of a) a polypeptide comprising an amino acid sequence selected from the group consisting of SEQ ID NO:253-506, b) a polypeptide comprising a naturally occurring amino acid sequence at least 90% identical to an amino acid sequence selected from the group consisting of SEQ ID NO:253-506, c) a biologically active fragment of a polypeptide having an amino acid sequence selected from the group consisting of SEQ ID NO:253-506, and d) an immunogenic fragment of a polypeptide having an amino acid sequence selected from the group consisting of SEQ ID NO:253-506, and a pharmaceutically acceptable excipient. In one embodiment, the composition comprises a polypeptide having an amino acid sequence selected from the group consisting of SEQ ID NO:253-506. The invention additionally provides a method of treating a disease or condition associated with decreased expression of functional MDDT, comprising administering to a patient in need of such treatment the composition.

The invention also provides a method for screening a compound for effectiveness as an agonist of a polypeptide selected from the group consisting of a) a polypeptide comprising an amino acid sequence selected from the group consisting of SEQ ID NO:253-506, b) a polypeptide comprising a naturally occurring amino acid sequence at least 90% identical to an amino acid sequence selected from the group consisting of SEQ ID NO:253-506, c) a biologically active fragment of a polypeptide having an amino acid sequence selected from the group consisting of SEQ ID NO:253-506, and d) an immunogenic fragment of a polypeptide having an amino acid sequence selected from the group consisting of SEQ ID NO:253-506. The method comprises a) exposing a sample comprising the polypeptide to a compound, and b) detecting agonist activity in the sample. In one alternative, the invention provides a composition comprising an agonist compound identified by the method and a pharmaceutically acceptable excipient. In another alternative, the invention provides a method of treating a disease or condition associated with decreased expression of functional MDDT, comprising administering to a patient in need of such treatment the composition.

Additionally, the invention provides a method for screening a compound for effectiveness as an antagonist of a polypeptide selected from the group consisting of a) a polypeptide comprising an amino acid sequence selected from the group consisting of SEQ ID NO:253-506, b) a polypeptide comprising a naturally occurring amino acid sequence at least 90% identical to an amino acid sequence selected from the group consisting of SEQ ID NO:253-506, c) a biologically active fragment of a polypeptide having an amino acid sequence selected from the group consisting of SEQ ID NO:253-506, and d) an immunogenic fragment of a polypeptide having an amino acid sequence selected from the group consisting of SEQ ID NO:253-506. The method comprises a) exposing a sample comprising the polypeptide to a compound, and b) detecting antagonist activity in the sample. In one alternative, the invention provides a composition comprising an antagonist compound identified by the method and a pharmaceutically acceptable excipient. In another alternative, the invention provides a method of treating a disease or condition associated with overexpression of functional MDDT, comprising administering to a patient in need of such treatment the composition.

The invention further provides a method of screening for a compound that modulates the activity of a polypeptide selected from the group consisting of a) a polypeptide comprising an amino acid sequence selected from the group consisting of SEQ ID NO:253-506, b) a polypeptide comprising a naturally occurring amino acid sequence at least 90% identical to an amino acid sequence selected from the group consisting of SEQ ID NO:253-506, c) a biologically active fragment of a polypeptide having an amino acid sequence selected from the group consisting of SEQ ID NO:253-506, and d) an immunogenic fragment of a polypeptide having an amino acid sequence selected from the group consisting of SEQ ID NO:253-506. The method comprises a) combining the polypeptide with at least one test compound under conditions permissive for the activity of the polypeptide. b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

### DESCRIPTION OF THE TABLES

Table 1 shows the sequence identification numbers (SEQ ID NO:s) and template identification numbers (template IDs) corresponding to the polynucleotides of the present invention, along with the sequence identification numbers (SEQ ID NO:s) and open reading frame identification numbers (ORF IDs) corresponding to polypeptides encoded by the template ID.

Table 2 shows the sequence identification numbers (SEQ ID NO:s) and template identification numbers (template IDs) corresponding to the polynucleotides of the present invention, along with their GenBank hits (GI Numbers), probability scores, and functional annotations corresponding to the GenBank hits.

Table 3 shows the sequence identification numbers (SEQ ID NO:s) and template identification numbers (template IDs) corresponding to the polynucleotides of the present invention, along with polynucleotide segments of each template sequence as defined by the indicated “start” and “stop” nucleotide positions. The reading frames of the polynucleotide segments and the Pfam hits. Pfam descriptions, and E-values corresponding to the polypeptide domains encoded by the polynucleotide segments are indicated.

Table 4 shows the sequence identification numbers (SEQ ID NO:s) and template identification numbers (template IDs) corresponding to the polynucleotides of the present invention, along with polynucleotide segments of each template sequence as defined by the indicated “start” and “stop” nucleotide positions. The reading frames of the polynucleotide segments are shown, and the polypeptides encoded by the polynucleotide segments constitute either signal peptide (SP) or transmembrane (TM) domains, as indicated. The membrane topology of the encoded polypeptide sequence is indicated, the N-terminus (N) listed as being oriented to either the cytosolic (N in) or non-cytosolic (N out) side of the cell membrane or organelle.

Table 5 shows the sequence identification numbers (SEQ ID NO:s) and template identification numbers (template IDs) corresponding to the polynucleotides of the present invention, along with component sequence identification numbers (component IDs) corresponding to each template. The component sequences, which were used to assemble the template sequences, are defined by the indicated “start” and “stop” nucleotide positions along each template.

Table 6 shows the tissue distribution profiles for the templates of the invention.

Table 7 shows the sequence identification numbers (SEQ ID NO:s) corresponding to the polypeptides of the present invention, along with the reading frames used to obtain the polypeptide segments, the lengths of the polypeptide segments, the “start” and “stop” nucleotide positions of the polynucleotide sequences used to define the encoded polypeptide segments, the GenBank hits (GI Numbers), probability scores, and functional annotations corresponding to the GenBank hits.

Table 8 summarizes the bioinformatics tools which are useful for analysis of the polynucleotides of the present invention. The first column of Table 8 lists analytical tools, programs, and algorithms, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score, the greater the homology between two sequences).

### DETAILED DESCRIPTION OF THE INVENTION

Before the nucleic acid sequences and methods are presented, it is to be understood that this invention is not limited to the particular machines, methods, and materials described. Although particular embodiments are described, machines, methods, and materials similar or equivalent to these embodiments may be used to practice the invention. The preferred makes, methods, and materials set forth are not intended to limit the scope of the invention which is limited only by the appended claims.

The singular forms “a”, “an”, and “the” include plural reference unless the context clearly dictates otherwise. All technical and scientific terms have the meanings commonly understood by one of ordinary skill in the art. All publications are incorporated by reference for the purpose of describing and disclosing the cell lines, vectors, and methodologies which are presented and which might be used in connection with the invention. Nothing in the specification is to be construed as an admission that the invention is not entitled to antedate such disclosure by virtue of prior invention.

Definitions

As used herein, the lower case “mddt” refers to a nucleic acid sequence, while the upper case “MDDT” refers to an amino acid sequence encoded by mddt. A “full-length” mddt refers to a nucleic acid sequence containing the entire coding region of a gene endogenously expressed in human tissue.

“Adjuvants” are materials such as Freund's adjuvant, mineral gels (aluminum hydroxide), and surface active substances (lysolecithin, pluronic polyols, polyanions, peptides, oil emulsions, keyhole limpet hemocyanin, and dinitrophenol) which may be administered to increase a host's immunological response.

“Allele” refers to an alternative form of a nucleic acid sequence. Alleles result from a “mutation,” a change or an alternative reading of the genetic code. Any given gene may have none, one, or many allelic forms. Mutations which give rise to alleles include deletions, additions, or substitutions of nucleotides. Each of these changes may occur alone, or in combination with the others, one or more times in a given nucleic acid sequence. The present invention encompasses allelic mddt.

“Amino acid sequence” refers to a peptide, a polypeptide, or a protein of either natural or synthetic origin. The amino acid sequence is not limited to the complete, endogenous amino acid sequence and may be a fragment, epitope, variant, or derivative of a protein expressed by a nucleic acid sequence.

“Amplification” refers to the production of additional copies of a sequence and is carried out using polymerase chain reaction (PCR) technologies well known in the art.

“Antibody” refers to intact molecules as well as to fragments thereof, such as Fab, F(ab′)₂, and Fv fragments, which are capable of binding the epitopic determinant. Antibodies that bind MDDT polypeptides can be prepared using intact polypeptides or using fragments containing small peptides of interest as the immunizing antigen. The polypeptide or peptide used to immunize an animal (e.g., a mouse, a rat, or a rabbit) can be derived from the translation of RNA, or synthesized chemically, and can be conjugated to a carrier protein if desired. Commonly used carrier that are chemically coupled to peptides include bovine serum albumin, thyroglobulin, and keyhole limpet hemocyanin (KLH). The coupled peptide is then used to immunize the animal.

“Antisense sequence” refers to a sequence capable of specifically hybridizing to a target sequence. The antisense sequence may include DNA, RNA, or any nucleic acid mimic or analog such as peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine.

“Antisense sequence” refers to a sequence capable of specifically hybridizing to a target sequence. The antisense sequence can be DNA, RNA, or any nucleic acid mimic or analog.

“Antisense technology” refers to any technology which relies on the specific hybridization of an antisense sequence to a target sequence.

A “bin” is a portion of computer memory space used by a computer program for storage of data, and bounded in such a manner that data stored in a bin may be retrieved by the program.

“Biologically active” refers to an amino acid sequence having a structural, regulatory, or biochemical function of a naturally occurring amino acid sequence.

“Clone joining” is a process for combining gene bins based upon the bins' containing sequence information from the same clone. The sequences may assemble into a primary gene transcript as well as one or more splice variants.

“Complementary” describes the relationship between two single-stranded nucleic acid sequences that anneal by base-pairing (5′-A-G-T-3′ pairs with its complement 3′-T-C-A-5′).

A “component sequence” is a nucleic acid sequence selected by a computer program such as PHRED and used to assemble a consensus or template sequence from one or more component sequences.

A “consensus sequence” or “template sequence” is a nucleic acid sequence which has been assembled from overlapping sequences, using a computer program for fragment assembly such as the GELVIEW fragment assembly system (Genetics Computer Group (GCG), Madison Wis.) or using a relational database management system (RDMS).

“Conservative amino acid substitutions” are those substitutions that, when made, least interfere with the properties of the original protein, i.e., the structure and especially the function of the protein is conserved and not significantly changed by such substitutions. The table below shows amino acids which may be substituted for an original amino acid in a protein and which are regarded as conservative substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative substitutions generally maintain (a) the structure of the polypeptide backbone in the area of the substitution, for example, as a beta sheet or alpha helical conformation, (b) the charge or hydrophobicity of the molecule at the target site, or (c) the bulk of the side chain.

“Deletion” refers to a change in either a nucleic or amino acid sequence in which at least one nucleotide or amino acid residue, respectively, is absent.

“Derivative” refers to the chemical modification of a nucleic acid sequence, such as by replacement of hydrogen by an alkyl, acyl, amino, hydroxyl, or other group.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

The terms “element” and “array element” refer to a polynucleotide, polypeptide, or other chemical compound having a unique and defined position on a microarray.

“E-value” refers to the statistical probability that a match between two sequences occurred by chance.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of mddt or MDDT which is identical in sequence to but shorter in length than the parent sequence. A fragment may comprise up 0 the entire length of the defined sequence, minus one nucleotide/amino acid residue. For example, a fragment may comprise from 10 to 1000 contiguous amino acid residues or nucleotides. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous amino acid residues or nucleotides in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing and the figures, may be encompassed by the present embodiments.

A fragment of mddt comprises a region of unique polynucleotide sequence that specifically identifies mddt, for example, as distinct from any other sequence in the same genome. A fragment of mddt is useful, for example, in hybridization and amplification technologies and in analogous methods that distinguish mddt from related polynucleotide sequences. The precise length of a fragment of mddt and the region of mddt to which the fragment corresponds are routinely determinable by one of ordinary skill in the art based on the intended purpose for the fragment.

A fragment of MDDT is encoded by a fragment of mddt. A fragment of MDDT comprises a region of unique amino acid sequence that specifically identifies MDDT. For example, a fragment of MDDT is useful as an immunogenic peptide for the development of antibodies that specifically recognize MDDT. The precise length of a fragment of MDDT and the region of MDDT to which the fragment corresponds are routinely determinable by one of ordinary skill in the art based on the intended purpose for the fragment.

A “full length” nucleotide sequence is one containing at least a start site for translation to a protein sequence, followed by an open reading frame and a stop site, and encoding a “full length” polypeptide.

“Hit” refers to a sequence whose annotation will be used to describe a given template. Criteria for selecting the top hit are as follows: if the template has one or more exact nucleic acid matches, the top hit is the exact match with highest percent identity. If the template has no exact matches but has significant protein hits, the top hit is the protein hit with the lowest E-value. If the template has no significant protein hits, but does have significant non-exact nucleotide hits, the top hit is the nucleotide hit with the lowest E-value.

“Homology” refers to sequence similarity either between a reference nucleic acid sequence and at least a fragment of an mddt or between a reference amino acid sequence and a fragment of an MDDT.

“Hybridization”refers to the process by which a strand of nucleotides anneals with a complementary strand through base pairing. Specific hybridization is an indication that two nucleic acid sequences share a high degree of identity. Specific hybridization complexes form under defined annealing conditions, and remain hybridized after the “washing” step. The defined hybridization conditions include the annealing conditions and the washing step(s), the latter of which is particularly important in determining the stringency of the hybridization process, with more stringent conditions allowing less non-specific binding, i.e., binding between pairs of nucleic acid probes that are not perfectly matched. Permissive conditions for annealing of nucleic acid sequences are routinely determinable and may be consistent among hybridization experiments, whereas wash conditions may be varied among experiments to achieve the desired stringency.

Generally, stringency of hybridization is expressed with reference to the temperature under which the wash step is carried out. Generally, such wash temperatures are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization is well known and can be found in Sambrook et al., 1989, Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., or 55° C. may be used. SSC concentration may be varied from about 0.2 to 2×SSC, with SDS being present at about 0.1%. Typically, blocking reagents are used to block non-specific hybridization. Such blocking reagents include, for instance, denatured salmon sperm DNA at about 100-200 μg/ml. Useful variations on these conditions will be readily apparent to those skilled in the art. Hybridization, particularly under high stringency conditions, may be suggestive of evolutionary similarity between the nucleotides. Such similarity is strongly indicative of a similar role for the nucleotides and their resultant proteins.

Other parameters, such as temperature, salt concentration, and detergent concentration may be varied to achieve the desired stringency. Denaturants, such as formamide at a concentration of about 35-50% v/v, may also be used under particular circumstances, such as RNA:DNA hybridizations. Appropriate hybridization conditions are routinely determinable by one of ordinary skill in the art.

“Immunologically active” or “immunogenic” describes the potential for a natural, recombinant, or synthetic peptide, epitope, polypeptide, or protein to induce antibody production in appropriate animals, cells, or cell lines.

“Insertion” or “assertion” refers to a change in either a nucleic or an amino acid sequence in which at least one nucleon or residue, respectively, is added to the sequence.

“Labeling” refers to the covalent or noncovalent joining of a polynucleotide, polypeptide, or antibody with a reporter molecule capable of producing a detectable or measurable signal.

“Microarray” is any arrangement of nucleic acids, amino acids, antibodies, etc., on a substrate. The substrate may be a solid support such as beads, glass, paper, nitrocellulose, nylon, or an appropriate membrane.

“Linkers” are short stretches of nucleotide sequence which may be added to a vector or an mddt to create restriction endonuclease sites to facilitate cloning. “Polylinkers” are engineered to incorporate multiple restriction enzyme sites and to provide for the use of enzymes which leave 5′ or 3′ overhangs (e.g., BamHI, EcoRI, and HindIII) and those which provide blunt ends (e.g., EcoRV, SnaBI, and StuI).

“Naturally occurring” refers to an endogenous polynucleotide or polypeptide that may be isolated from viruses or prokaryotic or eukaryotic cells.

“Nucleic acid sequence” refers to the specific order of nucleotides joined by phosphodiester bonds in a linear, polymeric arrangement. Depending on the number of nucleotides, the nucleic acid sequence can be considered an oligomer, oligonucleotide, or polynucleotide. The nucleic acid can be DNA, RNA, or any nucleic acid analog, such as PNA, may be of genomic or synthetic origin, may be either double-stranded or single-stranded, and can represent either the sense or antisense (complementary) strand.

“Oligomer” refers to a nucleic acid sequence of at least about 6 nucleotides and as many as about 60 nucleotides, preferably about 15 to 40 nucleotides, and most preferably between about 20 and 30 nucleotides, that may be used in hybridization or amplification technologies. Oligomers may be used as, e.g., primers for PCR, and are usually chemically synthesized.

“Operably linked” refers to the situation in which a first nucleic acid sequence is placed in a functional relationship with the second nucleic acid sequence. For instance, a promoter is operably linked to a coding sequence if the promoter affects the transcription or expression of the coding sequence. Generally, operably linked DNA sequences may be in close proximity or contiguous and, where necessary to join two protein coding regions, in the same reading frame.

“Peptide nucleic acid” (PNA) refers to a DNA mimic in which nucleotide bases are attached to a pseudopeptide backbone to increase stability. PNAs, also designated antigene agents, can prevent gene expression by targeting complementary messenger RNA.

The phrases “percent identity” and “% identity”, as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and Sharp, P. M. (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequence pairs.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to determine alignment between a known polynucleotide sequence and other sequences on a variety of databases. Also available is a tool called “BLAST 2 Sequences” that is used for direct pairwise comparison of two nucleotide sequences. “BLAST 2 Sequences” can be accessed and used interactively at http://www.ncbi.nim.nih.gov/gorf/bl2/. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.9 (May-07-1999) set at default parameters. Such default parameters may be, for example:

Matrix: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in figures or Sequence Listings, may be used to describe a length over which percentage identity may be measured.

Nucleic acid sequences that do not show a high degree of identity may nevertheless encode similar amino acid sequences due to the degeneracy of the genetic code. It is understood that changes in nucleic acid sequence can be made using this degeneracy to produce multiple nucleic acid sequences that all encode substantially the same protein.

The phrases “percent identity” and “% identity”, as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the hydrophobicity and acidity of the substituted residue, thus preserving the structure (and therefore function) of the folded polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.9 (May-07-1999) with blastp set at default parameters. Such default parameters may be, for example:

Matrix: BLOSUM62

Open Gap: 11 and Extension Gap: 1 penalty

Gap x drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in figures or Sequence listings, may be used to describe a length over which percentage identity may be measured.

“Post-translational modification” of an MDDT may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu and the MDDT.

“Probe” refers to mddt or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the figures and Sequence Listing, may be used.

Methods for preparing and using probes and primers are described in the references, for example Sambrook et al., 1989, Molecular Cloning: A Laboratory Manual, 2^{nd} ed., vol. 1-3, Cold Spring Harbor Press, Plainview N.Y.; Ausubel et al.,1987, Current Protocols in Molecular Biology, Greene Publ. Assoc. & Wiley-Intersciences, New York N.Y.; Innis et al., 1990, PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/ [symbol] Center for Genome Research, Cambridge Mass.) allows the user to input a “mispriming library.” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

“Purified” refers to molecules, either polynucleotides or polypeptides that are isolated or separated from their natural environment and are at least 60% free, preferably at least 75% free, and most preferably at least 90% free from other compounds with which they are naturally associated.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

“Regulatory element” refers to a nucleic acid sequence from nontranslated regions of a gene, and includes enhancers, promoters, introns, and 3′ untranslated regions, which interact with host proteins to carry out or regulate transcription or translation.

“Reporter” molecules are chemical or biochemical moieties used for labeling a nucleic acid, an amino acid, or an antibody. They include radionuclides; enzymes; fluorescent, chemiluminescent, or chromogenic agents; substrates; cofactors; inhibitors; magnetic particles; and other moieties known in the art.

An “RNA equivalent” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

“Sample” is used in its broadest sense. Samples may contain nucleic or amino acids, antibodies, or other materials, and may be derived from any source (e.g., bodily fluids including, but not limited to, saliva, blood, and urine; chromosome(s), organelles, or membranes isolated from a cell; genomic DNA, RNA, or cDNA in solution or bound to a substrate; and cleared cells or tissues or blots or imprints from such cells or tissues).

“Specific binding” or “specifically binding” refers to the interaction between a protein or peptide and its agonist, antibody, antagonist, or other binding partner. The interaction is dependent upon the presence of a particular structure of the protein, e.g., the antigenic determinant or epitope, recognized by the binding molecule. For example, if an antibody is specific for epitope “A,” the presence of a polypeptide containing epitope A, or the presence of free unlabeled A, in a reaction containing free labeled A and the antibody will reduce the amount of labeled A that binds to the antibody.

“Substitution” refers to the replacement of at least one nucleotide or amino acid by a different nucleotide or amino acid.

“Substrate” refers to any suitable rigid or semi-rigid support including, e.g., membranes, filters, chips, slides, wafers, fibers, magnetic or nonmagnetic beads, gels, tubing, plates, polymers, microparticles or capillaries. The substrate can have a variety of surface forms, such as wells, trenches, pins, channels and pores, to which polynucleotides or polypeptides are bound.

A “transcript image” refers to the collective pattern of gene expression by a particular tissue or cell type under given conditions at a given time.

“Transformation” refers to a process by which exogenous DNA enters a recipient cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed.

“Transformants” include stably transformed cells in which the inserted DNA is capable of replication either as an autonomously replicating plasmid or as part of the host chromosome, as well as cells which transiently express inserted DNA or RNA.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduced into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, and plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 25% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 30%, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length. The variant may result in “conservative” amino acid changes which do not affect structural and/or chemical properties. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

In an alternative, variants of the polynucleotides of the present invention may be generated through recombinant methods. One possible method is a DNA shuffling technique such as MOLECULARBREEDING (Maxygen Inc., Santa Clara Calif.; described in U.S. Pat. No. 5,837,458; Chang, C. -C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of MDDT, such as its biological or enzymatic activity or its ability to bind to other molecules or compounds. DNA shuffling is a process by which a library of gene variants is produced using PCR-method recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May-07-1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

### THE INVENTION

In a particular embodiment, cDNA sequences derived from human tissues and cell lines were aligned based on nucleotide sequence identity and assembled into “consensus” or “template” sequences which are designated by the template identification numbers (template IDs) in column 2 of Table 2. The sequence identification numbers (SEQ ID NO:s) corresponding to the template IDs are shown in column 1. The template sequences have similarity to GenBank sequences, or “hits,” as designated by the GI Numbers in column 3. The statistical probability of each GenBank hit is indicated by a probability score in column 4, and the functional annotation corresponding to each GenBank hit is listed in column 5.

The invention incorporates the nucleic acid sequences of these templates as disclosed in the Sequence Listing and the use of these sequences in the diagnosis and treatment of disease states characterized by defects in disease detection and treatment molecules. The invention further utilizes these sequences in hybridization and amplification technologies, and in particular, in technologies which assess gene expression patterns correlated with specific cells or tissues and their responses in vivo or in vitro to pharmaceutical agents, toxins, and other treatments. In this manner, the sequences of the present invention are used to develop a transcript image for a particular cell or tissue.

Derivation of Nucleic Acid Sequences

cDNA was isolated from libraries constructed using RNA derived from normal and diseased human tissues and cell lines. The human tissues and cell lines used for cDNA library construction were selected from a broad range of sources to provide a diverse population of cDNAs representative of gene transcription throughout the human body. Descriptions of the human tissues and cell lines used for cDNA library construction are provided in the LIFESEQ database (Incyte Genomics, Inc. (Incyte), Palo Alto Calif.). Human tissues were broadly selected from, for example, cardiovascular, dermatologic, endocrine, gastrointestinal, hematopoietic/immune system, musculoskeletal, neural, reproductive, and urologic sources.

Cell lines used for cDNA library construction were derived from, for example, leukemic cells, teratocarcinomas, neuroepitheliomas, cervical carcinoma, lung fibroblasts, and endothelial cells. Such cell lines include, for example, THP-1, Jurkat, HUVEC, hNT2, WI38, HeLa, and other cell lines commonly used and available from public depositories (American Type Culture Collection, Manassas Va.). Prior to mRNA isolation, cell lines were untreated, treated with a pharmaceutical agent such as 5′-aza-2′-deoxycytidine, treated with an activating agent such as lipopolysaccharide in the case of leukocytic cell lines, or, in the case of endothelial cell lines, subjected to shear stress.

Sequencing of the cDNAs

Methods for DNA sequencing are well known in the art. Conventional enzymatic methods employ the Klenow fragment of DNA polymerase I, SEQUENASE DNA polymerase (U.S. Biochemical Corporation, Cleveland Ohio), Taq polymerase (Applied Biosystems, Foster City Calif.), thermostable T7 polymerase (Amersham Pharmacia Biotech, Inc. (Amersham Pharmacia Biotech), Piscataway N.J.), or combinations of polymerases and proofreading exonucleases such as those found in the ELONGASE amplification system (Life Technologies Inc. (Life Technologies), Gaithersburg Md.), to extend the nucleic acid sequence from an oligonucleotide primer annealed to the DNA template of interest. Methods have been developed for the use of both single-stranded and double-stranded templates. Chain termination reaction products may be electrophoresed on urea-polyacrylamide gels and detected either by autoradiography (for radioisotope-labeled nucleotides) or by fluorescence (for fluorophore-labeled nucleotides). Automated methods for mechanized reaction preparation, sequencing, and analysis using fluorescence detection methods have been developed. Machines used to prepare cDNAs for sequencing can include the MICROLAB 2200 liquid transfer system (Hamilton Company (Hamilton), Reno Nev.), Peltier thermal cycler (PTC200; MJ Research, Inc. (MJ Research), Watertown Mass.), and ABI CATALYST 800 thermal cycler (Applied Biosystems). Sequencing can be carried out using, for example, the ABI 373 or 377 (Applied Biosystems) or MEGABACE 1000 (Molecular Dynamics, Inc. (Molecular Dynamics), Sunnyvale Calif.) DNA sequencing systems, or other automated and manual sequencing systems well known in the art.

The nucleotide sequences of the Sequence Listing have been prepared by current, state-of-the-art, automated methods and, as such, may contain occasional sequencing errors or unidentified nucleotides. Such unidentified nucleotides are designated by an N. These infrequent unidentified bases do not represent a hindrance to practicing the invention for those skilled in the art. Several methods employing standard recombinant techniques may be used to correct errors and complete the missing sequence information. (See, e.g., those described in Ausubel, F. M. et al. (1997) Short Protocols in Molecular Biology, John Wiley & Sons, New York N.Y.; and Sambrook, J. et al. (1989) Molecular Cloning, A Laboratory Manual, Cold Spring Harbor Press, Plainview N.Y.)

Assembly of cDNA Sequences

Human polynucleotide sequences may be assembled using programs or algorithms well known in the art. Sequences to be assembled are related, wholly or in part, and may be derived from a single or many different transcripts. Assembly of the sequences can be performed using such programs as PHRAP (Phils Revised Assembly Program) and the GELVIEW fragment assembly system (GCG), or other methods known in the art.

Alternatively, cDNA sequences are used as “component” sequences that are assembled into “template” or “consensus” sequences as follows. Sequence chromatograms are processed, verified, and quality scores are obtained using PHRED. Raw sequences are edited using an editing pathway known as Block 1 (See, e.g., the LIFESEQ Assembled User Guide, Incyte Genomics, Palo Alto, Calif.). A series of BLAST comparisons is performed and low-information segments and repetitive elements (e.g., dinucleotide repeats, Alu repeats, etc.) are replaced by “n's”, or masked, to prevent spurious matches. Mitochondrial and ribosomal RNA sequences are also removed. The processed sequences are then loaded into a relational database management system (RDMS) which assigns edited sequences to existing templates, if available. When additional sequences are added into the RDMS, a process is initiated which modifies existing templates or creates new templates from works in progress (i.e., nonfinal assembled sequences) containing queued sequences or the sequences themselves. After the new sequences have been assigned to templates, the templates can be merged into bins. If multiple templates exist in one bin, the bin can be split and the templates reannotated.

Once gene bins have been generated based upon sequence alignments, bins are “clone joined” based upon clone information. Clone joining occurs when the 5′ sequence of one clone is present in one bin and the 3′ sequence from the same clone is present in a different bin, indicating that the two bins should be merged into a single bin. Only bins which share at least two different clones are merged.

A resultant template sequence may contain either a partial or a full length open reading frame, or all or part of a genetic regulatory element. This variation is due in part to the fact that the full length cDNAs of many genes are several hundred, and sometimes several thousand, bases in length. With current technology, cDNAs comprising the coding regions of large genes cannot be cloned because of vector limitations, incomplete reverse transcription of the mRNA, or incomplete “second strand” synthesis. Template sequences may be extended to include additional contiguous sequences derived from the parent RNA transcript using a variety of methods known to those of skill in the art. Extension may thus be used to achieve the full length coding sequence of a gene.

Analysis of the cDNA Sequences

The cDNA sequences are analyzed using a variety of programs and algorithms which are well known in the art. (See, e.g., Ausubel, 1997, supra, Chapter 7.7; Meyers, R. A. (Ed.) (1995) Molecular Biology and Biotechnology, Wiley VCH, New York N.Y., pp. 856-853; and Table 8.) These analyses comprise both reading frame determinations, e.g., based on triplet codon periodicity for particular organisms (Fickett, J. W. (1982) Nucleic Acids Res. 10:5303-5318); analyses of potential start and stop codons; and homology searches.

Computer programs known to those of skill in the art for performing computer-assisted searches for amino acid and nucleic acid sequence similarity, include, for example, Basic Local Alignment Search Tool (BLAST; Altschul, S. F. (1993) J. Mol. Evol. 36:290-300; Altschul, S. F. et al. (1990) J. Mol. Biol. 215:403-410). BLAST is especially useful in determining exact matches and comparing two sequence fragments of arbitrary but equal lengths, whose alignment is locally maximal and for which the alignment score meets or exceeds a threshold or cutoff score set by the user (Karlin, S. et al. (1988) Proc. Natl. Acad. Sci. USA 85:841-845). Using an appropriate search tool (e.g., BLAST or HMM), GenBank, SwissProt, BLOCKS, PFAM and other databases may be searched for sequences containing regions of homology to a query mddt or MDDT of the present invention.

Other approaches to the identification, assembly, storage, and display of nucleotide and polypeptide sequences are provided in “Relational Database for Storing Biomolecule Information,” U.S. Ser. No. 08/947,845, filed Oct. 9, 1997; “Project-Based Full-Length Biomolecular Sequence Database,” U.S. Ser. No. 08/811,758, filed Mar. 6, 1997; and “Relational Database and System for Storing Information Relating to Biomolecular Sequences,” U.S. Ser. No. 09/034,807, filed Mar. 4, 1998, all of which are incorporated by reference herein in their entirety.

Protein hierarchies can be assigned to the putative encoded polypeptide based on, e.g., motif, BLAST, or biological analysis. Methods for assigning these hierarchies are described, for example, in “Database System Employing Protein Function Hierarchies for Viewing Biomolecular Sequence Data,” U.S. Ser. No. 08/812,290, filed Mar. 6, 1997, incorporated herein by reference.

Human Disease Detection and Treatment Molecule Sequences

The mddt of the present invention may be used for a variety of diagnostic and therapeutic purposes. For example, an mddt may be used to diagnose a particular condition, disease, or disorder associated with disease detection and treatment molecules. Such conditions, diseases, and disorders include, but are not limited to, a cell proliferative disorder, such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; and an autoimmune/inflammatory disorder, such as actinic keratosis, acquired immunodeficiency syndrome (AIDS), Addison's disease, adult respiratory distress syndrome, allergies, ankylosing spondylitis, amyloidosis, anemia, arteriosclerosis, asthma, atherosclerosis, autoimmune hemolytic anemia, autoimmune thyroiditis, bronchitis, bursitis, cholecystitis, cirrhosis, contact dermatitis, Crohn's disease, atopic dermatitis, dermatomyositis, diabetes mellitus, emphysema, erythroblastosis fetalis, erythema nodosum, atrophic gastritis, glomerulonephritis, Goodpasture's syndrome, gout, Graves' disease, Hashimoto's thyroiditis, paroxysmal nocturnal hemoglobinuria, hepatitis, hypereosinophilia, irritable bowel syndrome, episodic lymphopenia with lymphocytotoxins, mixed connective tissue disease (MCTD), multiple sclerosis, myasthenia gravis, myocardial or pericardial inflammation, myelofibrosis, osteoarthritis, osteoporosis, pancreatitis, polycythemia vera, polymyositis, psoriasis, Reiter's syndrome, rheumatoid arthritis, scleroderma, Sjögren's syndrome, systemic anaphylaxis, systemic lupus erythematosus, systemic sclerosis, primary thrombocythemia, thrombocytopenic purpura, ulcerative colitis, uveitis. Werner syndrome, complications of cancer, hemodialysis, and extracorporeal circulation, trauma, and hematopoietic cancer including lymphoma, leukemia, and myeloma. The mddt can be used to detect the presence of, or to quantify the amount of, an mddt-related polynucleotide in a sample. This information is then compared to information obtained from appropriate reference samples, and a diagnosis is established. Alternatively, a polynucleotide complementary to a given mddt can inhibit or inactivate a therapeutically relevant gene related to the mddt.

Analysis of mddt Expression Patterns

The expression of mddt may be routinely assessed by hybridization-based methods to determine, for example, the tissue-specificity, disease-specificity, or developmental stage-specificity of mddt expression. For example, the level of expression of mddt may be prepared among different cell types or tissues, among diseased and normal cell types or tissues, among cell types or tissues at different developmental stages, or among cell types or tissues undergoing various treatments. This type of analysis is useful, for example, to assess the relative levels of mddt expression in fully or partially differentiated cells or tissues, to determine if changes in mddt expression levels are correlated with the development or progression of specific disease states, and to assess the response of a cell or tissue to a specific therapy, for example, in pharmacological or toxicological studies. Methods for the analysis of mddt expression are based on hybridization and amplification technologies and include membrane-based procedures such as northern blot analysis, high-throughput procedures that utilize, for example, microarrays, and PCR-based procedures.

Hybridization and Genetic Analysis

The mddt, their fragments, or complementary sequences, may be used to identify the presence of and/or to determine the degree of similarity between two (or more) nucleic acid sequences. The mddt may be hybridized to naturally occurring or recombinant nucleic acid sequences under appropriately selected temperatures and salt concentrations. Hybridization with a probe based on the nucleic acid sequence of at least one of the mddt allows for the detection of nucleic acid sequences, including genomic sequences, which are identical or related to the mddt of the Sequence Listing. Probes may be selected from non-conserved or unique regions of at least one of the polynucleotides of SEQ ID NO:1-252 and tested for their ability to identify or amplify the target nucleic acid sequence using standard protocols.

Polynucleotide sequences that are capable of hybridizing, in particular, to those shown in SEQ ID NO:1-252 and fragments thereof, can be identified using various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions are discussed in “Definitions.”

A probe for use in Southern or northern hybridization may be derived from a fragment of an mddt sequence, or its complement, that is up to several hundred nucleotides in length and is either single-stranded or double-stranded. Such probes may be hybridized in solution to biological materials such as plasmids, bacterial, yeast, or human artificial chromosomes, cleared or sectioned tissues, or to artificial substrates containing mddt. Microarrays are particularly suitable for identifying the presence of and detecting the level of expression for multiple genes of interest by examining gene expression correlated with, e.g., various stages of development, treatment with a drug or compound, or disease progression. An array analogous to a dot or slot blot may be used to arrange and link polynucleotides to the surface of a substrate using one or more of the following: mechanical (vacuum), chemical, thermal, or UV bonding procedures. Such an array may contain any number of mddt and may be produced by hand or by [symbol] available devices, materials, and machines.

Microarrays may prepared, used, and analyzed using methods own in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.)

Probes may be labeled by either PCR or enzymatic techniques using a variety of commercially available reporter molecules. For example, commercial kits are available for radioactive and chemiluminescent labeling (Amersham Pharmacia Biotech) and for alkaline phosphatase labeling (Life Technologies). Alternatively, mddt may be cloned into commercially available vectors for the production of RNA probes. Such probes may be transcribed in the presence of at least one labeled nucleotide (e.g., ³²P-ATP, Amersham Pharmacia Biotech).

Additionally the polynucleotides of SEQ ID NO:1-252 or suitable fragments thereof can be used to isolate full length cDNA sequences utilizing hybridization and/or amplification procedures well known in the art, e.g., cDNA library screening, PCR amplification, etc. The molecular cloning of such full length cDNA sequences may employ the method of cDNA library screening with probes using the hybridization, stringency, washing, and probing strategies described above and in Ausubel, supra, Chapters 3, 5, and 6. These procedures may also be employed with genomic libraries to isolate genomic sequences of mddt in order to analyze, e.g., regulatory elements.

Genetic Mapping

Gene identification and mapping are important in the investigation and treatment of almost all conditions, diseases, and disorders. Cancer, cardiovascular disease, Alzheimer's disease, arthritis, diabetes, and mental illnesses are of particular interest. Each of these conditions is more complex than the single gene defects of sickle cell anemia or cystic fibrosis, with select groups of genes being predictive of predisposition for a particular condition, disease, or disorder. For example, cardiovascular disease may result from malfunctioning receptor molecules that fail to clear cholesterol from the bloodstream, and diabetes may result when a particular individual's immune system is activated by an infection and attacks the insulin-producing cells of the pancreas. In some studies, Alzheimer's disease has been linked to a gene on chromosome 21; other studies predict a different gene and location. Mapping of disease genes is a complex and reiterative process and generally proceeds from genetic linkage analysis to physical mapping.

As a condition is noted among members of a family, a genetic linkage map traces parts of chromosomes that are inherited in the same pattern as the condition. Statistics link the inheritance of particular conditions to particular regions of chromosomes, as defined by RFLP or other markers. (See, for example, Lande [symbol] S. and Botstein, D. (1986) Proc. Natl. Acad. Sci. USA 83:7353-7357.) Occasionally, genetic markers and their locations are known from previous studies. More often, however, the markers are simply stretches of DNA that differ among individuals. Examples of genetic linkage maps can be found in various scientific journals or at the Online Mendelian Inheritance in Man (OMIM) World Wide Web site.

In another embodiment of the invention, mddt sequences may be used to generate hybridization probes useful in chromosomal mapping of naturally occurring genomic sequences. Either coding or noncoding sequences of mddt may be used, and in some instances, noncoding sequences may be preferable over coding sequences. For example, conservation of an mddt coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.)

Fluorescent in situ hybridization (FISH) may be correlated with other physical chromosome mapping techniques and genetic map data. (See, e.g., Meyers, supra, pp. 965-968.) Correlation between the location of mddt on a physical chromosomal map and a specific disorder, or a predisposition to a specific disorder, may help define the region of DNA associated with that disorder. The mddt sequences may also be used to detect polymorphisms that are genetically linked to the inheritance of a particular condition, disease, or disorder.

In situ hybridization of chromosomal preparations and genetic mapping techniques, such as linkage analysis using established chromosomal markers, may be used for extending existing genetic maps. Often the placement of a gene on the chromosome of another mammalian species, such as mouse, may reveal associated markers even if the number or arm of the corresponding human chromosome is not known. These new marker sequences can be mapped to human chromosomes and may provide valuable information to investigators searching for disease genes using positional cloning or other gene discovery techniques. Once a disease or syndrome has been crudely correlated by genetic linkage with a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequences of the subject invention may also be used to detect differences in chromosomal architecture due to translocation, inversion, etc., among normal, carrier, or affected individuals.

Once a disease-associated gene is mapped to a chromosomal region, the gene must be cloned in order to identify mutant or other alterations (e.g., translocations o [symbol] esions) that may be correlated with disease. This process requires a physical map of the chromosomal region containing the disease-gene of interest along with associated markers. A physical map is necessary for determining the nucleotide sequence of and order of marker genes on a particular chromosomal region. Physical mapping techniques are well known in the art and require the generation of overlapping sets of cloned DNA fragments from a particular organelle, chromosome, or genome. These clones are analyzed to reconstruct and catalog their order. Once the position of a marker is determined, the DNA from that region is obtained by consulting the catalog and selecting clones from that region. The gene of interest is located through positional cloning techniques using hybridization or similar methods.

Diagnostic Uses

The mddt of the present invention may be used to design probes useful in diagnostic assays. Such assays, well known to those skilled in the art, may be used to detect or confirm conditions, disorders, or diseases associated with abnormal levels of mddt expression. Labeled probes developed from mddt sequences are added to a sample under hybridizing conditions of desired stringency. In some instances, mddt, or fragments or oligonucleotides derived from mddt, may be used as primers in amplification steps prior to hybridization. The amount of hybridization complex formed is quantified and compared with standards for that cell or tissue. If mddt expression varies significantly from the standard, the assay indicates the presence of the condition, disorder, or disease. Qualitative or quantitative diagnostic methods may include northern, dot blot, or other membrane or dip-stick based technologies or multiple-sample format technologies such as PCR, enzyme-linked immunosorbent assay (ELISA)-like, pin, or chip-based assays.

The probes described above may also be used to monitor the progress of conditions, disorders, or diseases associated with abnormal levels of mddt expression, or to evaluate the efficacy of a particular therapeutic treatment. The candidate probe may be identified from the mddt that are specific to a given human tissue and have not been observed in GenBank or other genome databases. Such a probe may be used in animal studies, preclinical tests, clinical trials, or in monitoring the treatment of an individual patient. In a typical process, standard expression is established by methods well known in the art for use as a basis of comparison, samples from patients affected by the disorder or disease are combined with the probe to evaluate any deviation from the standard profile, and a therapeutic agent is administered and effects are monitored to generate a treatment profile. Efficacy is evaluated by determining whether the expression progresses toward or returns to the standard normal pattern. Treatment profiles may be generated over a period of several days or several months. Statistical methods well known to those skilled in the art may be use to determine the significance of such therapeutic agents.

The polynucleotides are also useful for identifying individuals from minute biological samples, for example, by matching the RFLP pattern of a sample's DNA to that of an individual's DNA. The polynucleotides of the present invention can also be used to determine the actual base-by-base DNA sequence of selected portions of an individual's genome. These sequences can be used to prepare PCR primers for amplifying and isolating such selected DNA, which can then be sequenced. Using this technique, an individual can be identified through a unique set of DNA sequences. Once a unique ID database is established for an individual, positive identification of that individual can be made from extremely small tissue samples.

In a particular aspect, oligonucleotide primers derived from the mddt of the invention may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from mddt are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (is SNP), are capable of identifying polymorphisms by comparing the sequences of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

DNA-based identification techniques are critical in forensic technology. DNA sequences taken from very small biological samples such as tissues, e.g., hair or skin, or body fluids, e.g., blood, saliva, semen, etc., can be amplified using, e.g., PCR, to identify individuals. (See, e.g., Erlich, H. (1992) PCR Technology, Freeman and Co., New York, N.Y.). Similarly, polynucleotides of the present invention can be used as polymorphic markers.

There is also a need for reagents capable of identifying the source of a particular tissue. Appropriate reagents can comprise, for example, DNA probes or primers prepared from the sequences of the present invention that are specific for particular tissues. Panels of such reagents can identify tissue by species and/or by organ type. In a similar fashion, these reagents can be used to screen tissue cultures for examination.

The polynucleotides of the present invention can also be used as molecular weight markers on nucleic acid gels or Southern blots, as diagnostic probes for the presence of a specific mRNA in a particular cell type, in the creation of subtracted cDNA libraries which aid in the discovery of novel polynucleotides, in selection and synthesis of oligomers for attachment to an array or other support, and as an antigen to elicit an immune response.

Disease Model Systems Using mddt

The mddt of the invention or their mammalian homologs may be “knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

The mddt of the invention may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

The mddt of the invention can also be used to create “knockin” humanized animals (pigs) or transgenic animals (mice or rats) to model human disease. With knockin technology, a region of mddt is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress mddt, resulting, e.g., in the secretion of MDDT in its milk, may also serve as a convenient source of that protein (Janne, J. et al. (1998) Biotechnol. Annu. Rev. 4:55-74).

Screening Assays

MDDT encoded by polynucleotides of the present invention may be used to screen for molecules that bind to or are bound by the encoded polypeptides. The binding of the polypeptide and the molecule may activate (agonist), increase, inhibit (antagonist), or decrease activity of the polypeptide or the bound molecule. Examples of such molecules include antibodies, oligonucleotides, proteins (e.g., receptors), or small molecules.

Preferably, the molecule is closely related to the natural ligand of the polypeptide, e.g., a ligand or fragment thereof, a natural substrate, or a structural or functional mimetic. (See, Coligan et al., (1991) Current Protocols in Immunology 1(2): Chapter 5.) Similarly, the molecule can be closely related to the natural receptor to which the polypeptide binds, or to at least a fragment of the receptor, e.g., the active site. In either case, the molecule can be rationally designed using known techniques. Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide or cell membrane fractions which contain the expressed polypeptide are then contacted with a test compound and binding, stimulation, or inhibition of activity of either the polypeptide or the molecule is analyzed.

An assay may simply test binding of a candidate compound to the polypeptide, wherein binding is detected by a fluorophore, radioisotope, enzyme conjugate, or other detectable label. Alternatively, the assay may assess binding in the presence of a labeled competitor.

Additionally, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay using, e.g., a monoclonal or polyclonal antibody, can measure polypeptide level in a sample. The antibody can measure polypeptide level by either binding, directly or indirectly, to the polypeptide or by competing with the polypeptide for a substrate.

All of the above assays can be used in a diagnostic or prognostic context. The molecules discovered using these assays can be used to treat disease or to bring about a particular result in a patient (e.g., blood vessel growth) by activating or inhibiting the polypeptide/molecule. Moreover, the assays can discover agents which may inhibit or enhance the production of the polypeptide from suitably manipulated cells or tissues.

Transcript Imaging and Toxicological Testing

Another embodiment relates to the use of mddt to develop a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity pertaining to disease detection and treatment molecules.

Transcript images which profile mddt expression may be generated using transcripts isolated from tissues, cell lines, biopsies, or other biological samples. The transcript image may thus reflect mddt expression in vivo, as in the case of a tissue or biopsy sample, or in vitro, as in the case of a cell line.

Transcript images which profile mddt expression may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and Anderson, N. L. (2000) Toxicol. Lett. 112-113:467-71, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nih.gov/oc/news/toxchip.htm.) Therefore, it is important and desirable in toxicological screening using toxicant signatures to include all expressed gene sequences.

In one embodiment, the toxicity of a test compound is assessed in treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another particular embodiment relates to the use of MDDT encoded by polynucleotides of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomic profile may also be generated using antibodies specific for MDDT to quantify the levels of MDDT expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-11; Mendoze, L. G. et al. (1999) Biotechniques 27:778-88). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson. N. L. and Seilhamer, J. (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the MDDT encoded by polynucleotides of the present invention.

In another embodiment, the toxicity of a test compound is assessed by treating a biological sample containing proteins with the test compound. Proteins from the biological sample are incubated with antibodies specific to the MDDT encoded by polynucleotides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Transcript images may be used to profile mddt expression in distinct tissue types. This process can be used to determine disease detection and treatment molecule activity in a particular tissue type relative to this activity in a different tissue type. Transcript images may be used to generate a profile of mddt expression characteristic of diseased tissue. Transcript images of tissues before and after treatment may be used for diagnostic purposes, to monitor the progression of disease, and to monitor the efficacy of drug treatments for diseases which affect the activity of disease detection and treatment molecules.

Transcript images of cell lines can be used to assess disease detection and treatment molecule activity and/or to identify cell lines that lack or misregulate this activity. Such cell lines may then be treated with pharmaceutical agents, and a transcript image following treatment may indicate the efficacy of these agents in restoring desired levels of this activity. A similar approach may be used to assess the toxicity of pharmaceutical agents as reflected by undesirable changes in disease detection and treatment molecule [symbol] ity. Candidate pharmaceutical agents may be evaluated by comparing their associated transcript images with those of pharmaceutical agents of known effectiveness.

Antisense Molecules

The polynucleotides of the present invention are useful in antisense technology. Antisense technology or therapy relies on the modulation of expression of a target protein through the specific binding of an antisense sequence to a target sequence encoding the target protein or directing its expression. (See, e.g., Agrawal, S., ed. (1996) Antisense Therapeutics, Humana Press Inc. Totawa N.J.; Alama, A. et al. (1997) Pharmacol. Res. 36(3): 171-178; Crooke, S. T. (1997) Adv. Pharmacol. 40:1-49; Sharma, H. W. and R. Narayanan (1995) Bioessays 17(12):1055-1063; and Lavrosky, Y. et al. (1997) Biochem. Mol. Med. 62(1):11-22.) An antisense sequence is a polynucleotide sequence capable of specifically hybridizing to at least a portion of the target sequence. Antisense sequences bind to cellular mRNA and/or genomic DNA, affecting translation and/or transcription. Antisense sequences can be DNA, RNA, or nucleic acid mimics and analogs. (See, e.g., Rossi, J. J. et al. (1991) Antisense Res. Dev. 1(3):285-288; Lee, R. et al. (1998) Biochemistry 37(3):900-1010; Pardridge, W. M. et al. (1995) Proc. Natl. Acad. Sci. USA 92(12):5592-5596; and Nielsen, P. E. and Haaima, G. (1997) Chem. Soc. Rev. 96:73-78.) Typically, the binding which results in modulation of expression occurs through hybridization or binding of complementary base pairs. Antisense sequences can also bind to DNA duplexes through specific interactions in the major groove of the double helix.

The polynucleotides of the present invention and fragments thereof can be used as antisense sequences to modify the expression of the polypeptide encoded by mddt. The antisense sequences can be produced ex vivo, such as by using any of the ABI nucleic acid synthesizer series (Applied Biosystems) or other automated systems known in the art. Antisense sequences can also be produced biologically, such as by transforming an appropriate host cell with an expression vector containing the sequence of interest. (See, e.g., Agrawal, supra.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E., et al. (1998) J. Allergy Clin. Immunol. 102(3):469-475; and Scanlon, K. J., et al. (1995) 9(13): 1288-1296.) Antisense sequences can also be introduced intracellularly through the use of viral vectors, such as retrovirus and adeno-associated virus vectors. (See, e.g., Miller, A. D. (1990) Blood 76:271; Ausubel, F. M. et al. (1995) Current Protocols in Molecular Biology, John Wiley & Sons, New York N.Y.; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, [symbol] 1995) Br. Med. Bull. 51(1):217-225; Boa [symbol] J. et al. (1998) J. Pharm. Sci. 87(11):1308-1315; and Morris, M. C. et al. (1997) Nucleic Acids Res. 25(14):2730-2736.)

Expression

In order to express a biologically active MDDT, the nucleotide sequences encoding MDDT or fragments thereof may be inserted into an appropriate expression vector. i.e., a vector which contains the necessary elements for transcriptional and translational control of the inserted coding sequence in a suitable host. Methods which are well known to those skilled in the art may be used to construct expression vectors containing sequences encoding MDDT and appropriate transcriptional and translational control elements. These methods include in vitro recombinant DNA techniques, synthetic techniques, and in vivo genetic recombination. (See, e.g., Sambrook, supra, Chapters 4, 8, 16, and 17; and Ausubel, supra, Chapters 9, 10, 13, and 16.)

A variety of expression vector/host systems may be utilized to contain and express sequences encoding MDDT. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal (mammalian) cell systems. (See, e.g., Sambrook, supra; Ausubel, 1995, supra, Van Heeke, G. and S. M. Schuster (1989) J. Biol. Chem. 264:5503-5509; Bitter, G. A. et al. (1987) Methods Enzymol. 153:516-544; Scorer, C. A. et al. (1994) Bio/Technology 12:181-184; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; Coruzzi, G. et al. (1984) EMBO J. 3:1671-1680; Broglie, R. et al. (1984) Science 224:838-843; Winter, J. et al. (1991) Results Probl. Cell Differ. 17:85-105; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356: Yu, M. et al., (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

For long term production of recombinant proteins in mammalian systems, stable expression of MDDT in cell lines is preferred. For example, sequences encoding MDDT can be transformed into cell lines using expression vectors which may contain viral origins of relation and/or endogenous expression elements and a selectable marker gene on the same or on a separate vector. Any number of selection systems may be used to recover transformed cell lines. (See, e.g., Wigler, M. et al. (1977) Cell 11:223-232; Lowy, I. et al. (1980) Cell 22:817-823.; Wigler, M. et al. (1980) Proc. Natl. Acad. Sci. USA 77:3567-3570; Colbere-Garapin, F. et al. (1981) J. Mol. Biol. 150:1-14; Hartman, S. C. and R. C. Mulligan (1988) Proc. Natl. Acad. Sci. USA 85:8047-8051; Rhodes, C. A. (1995) Methods Mol. Biol. 55:121-131.)

Therapeutic Uses of mddt

The mddt of the invention may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassemias, familial hypercholesterolemia, and hemophilia resulting from Factor VIII or Factor IX deficiencies (Crystal, R. G. (1995) Science 270:404-410; Verma, I. M. and Somia, N. (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore. D. (1988) Nature 335:395-396; Poeschla, E. et al. (1996) Proc. Natl. Acad. Sci. USA. 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in mddt expression or regulation causes disease, the expression of mddt from an appropriate population of transduced cells may alleviate the clinical manifestations caused by the genetic deficiency.

In a further embodiment of the invention, diseases or disorders caused by deficiencies in mddt are treated by constructing mammalian expression vectors comprising mddt and introducing these vectors by mechanical means into mddt-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and Anderson, W. F. (1993) Annu. Rev. Biochem. 62:191-217; Ivics, Z. (1997) Cell 91:501-510; Boulay, J-L. and Récipon, H. (1998) Curr. Opin. Biotechnol. 9:445-450).

Expression vector that may be effective for the expression of may include, but are not limited to, the PCDNA 3.1, EPITAG, PRCCMV2, PREP, PVAX vectors (Invitrogen, Carlsbad Calif.). PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech. Palo Alto Calif.). The mddt of the invention may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and Bujard, H. (1992) Proc. Natl. Acad. Sci. U.S.A. 89:5547-5551; Gossen, M. et al., (1995) Science 268:1766-1769; Rossi, F. M. V. and Blau, H. M. (1998) Curr. Opin. Biotechnol. 9:451-456), commercially available in the T-REX plasmid (Invitrogen); the ecdysone-inducible promoter (available in the plasmids PVGRXR and PIND: Invitrogen); the FK506/rapamycin inducible promoter; or the RU486/mifepristone inducible promoter (Rossi, F. M. V. and Blau, H. M. supra), or (iii) a tissue-specific promoter or the native promoter of the endogenous gene encoding MDDT from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and Eb, A. J. (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to mddt expression are treated by constructing a retrovirus vector consisting of (i) mddt under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. U.S.A. 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and Miller, A. D. (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g. CD4⁺T-cells), and the return of transduced [symbol] to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:47074716; Ranga, U. et al. (1998) Proc. Natl. Acad. Sci. U.S.A. 95:1201-1206; Su, L. (1997) Blood 89:2283-2290).

In the alternative, an adenovirus-based gene therapy delivery system is used to deliver mddt to cells which have one or more genetic abnormalities with respect to the expression of mddt. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and Somia, N. (1997) Nature 18:389:239-242, both incorporated by reference herein.

In another alternative, a herpes-based, gene therapy delivery system is used to deliver mddt to target cells which have one or more genetic abnormalities with respect to the expression of mddt. The use of herpes simplex virus (HSV)-based vectors may be especially valuable for introducing mddt to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. 1999 J. Virol. 73:519-532 and Xu, H. et al., (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver mddt to target cells. The biology of the prototypic alphavirus, Semliki Forest Virus (SFV), has been studied extensively and gene transfer vectors have been based on the SFV genome (Garoff, H. and Li, K-J. (1998) Current Biotech. 9:464-469). During alphavirus RNA replication, a subgenomic RNA is generated that normally encodes the viral capsid proteins. This subgenomic RNA replicates to higher levels than the full-length genomic RNA, resulting in the overproduction of capsid proteins relative to the viral proteins with enzymatic activity (e.g., protease and polymerase). Similarly, inserting mddt into the alphavirus genome in place of the capsid-coding region results in the production of a large number of mddt RNAs and the synthesis of high levels of MDDT in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of mddt into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Antibodies

Anti-MDDT antibodies may be used to analyze protein expression levels. Such antibodies include, but are not limited to, polyclonal, monoclonal, chimeric, single chain, and Fab fragments. For descriptions of and protocols of antibody technologies, see, e.g., Pound J. D. (1998) Immunochemical Protocols, Humana Press, Totowa, N.J.

The amino acid sequence encoded by the mddt of the Sequence Listing may be analyzed by appropriate software (e.g., LASERGENE NAVIGATOR software, DNASTAR) to determine regions of high immunogenicity. The optimal sequences for immunization are selected from the C-terminus, the N-terminus, and those intervening, hydrophilic regions of the polypeptide which are likely to be exposed to the external environment when the polypeptide is in its natural conformation. Analysis used to select appropriate epitopes is also described by Ausubel (1997, supra, Chapter 11.7). Peptides used for antibody induction do not need to have biological activity; however, they must be antigenic. Peptides used to induce specific antibodies may have an amino acid sequence consisting of at least five amino acids, preferably at least 10 amino acids, and most preferably at least 15 amino acids. A peptide which mimics an antigenic fragment of the natural polypeptide may be fused with another protein such as keyhole hemolimpet cyanin (KLH; Sigma, St. Louis Mo.) for antibody production. A peptide encompassing an antigenic region may be expressed from an mddt, synthesized as described above, or purified from human cells.

Procedures well known in the art may be used for the production of antibodies. Various hosts including mice, goats, and rabbits, may be immunized by injection with peptide. Depending on the host species, various adjuvants may be used to increase immunological response.

In one procedure, peptides about 15 residues in length may be synthesized using an ABI 431 A peptide synthesizer (Applied Biosystems) using fmoc-chemistry and coupled to KLH (Sigma) by reaction with M-maleimidobenzoyl-N-hydroxysuccinimide ester (Ausubel, 1995, supra). Rabbits are immunized with the peptide-KLH complex in complete Freund's adjuvant. The resulting antisera are tested for antipeptide activity by binding the peptide to plastic, blocking with 1% bovine serum albumin (BSA), reacting with rabbit antisera, washing, and reacting with radioiodinated goat anti-rabbit IgG. Antisera with antipeptide activity are tested for anti-MDDT activity using protocols well known in the art, including ELISA, radioimmunoassay (RIA), and immunoblotting.

In another procedure, isolated and purified peptide may be used to immunize mice (about 100 μg of peptide) or rabbits (about 1 mg of peptide). Subsequently, the peptide is radioiodinated and used to screen the immunized animals' B-lymphocytes for production of antipeptide antibodies. Positive cells are then used to produce hybridomas using standard techniques. About 20 mg of peptide is sufficient for labeling and screening several thousand clones. Hybridomas of interest are detected by screening with radioiodinated peptide to identify those fusions producing peptide-specific monoclonal antibody. In a typical protocol, wells of a multi-well plate (FAST, Becton-Dickinson, Palo Alto, Calif.) are coated with affinity-purified, specific rabbit-anti-mouse (or suitable anti-species IgG) antibodies at 10 mg/ml. The coated wells are blocked with 1% BSA and washed and exposed to supernatants from hybridomas. After incubation, the wells are exposed to radiolabeled peptide at 1 mg/ml.

Clones producing antibodies bind a quantity of labeled peptide that is detectable above background. Such clones are expanded and subjected to 2 cycles of cloning. Cloned hybridomas are injected into pristane-treated mice to produce ascites, and monoclonal antibody is purified from the ascitic fluid by affinity chromatography on protein A (Amersham Pharmacia Biotech). Several procedures for the production of monoclonal antibodies, including in vitro production, are described in Pound (supra). Monoclonal antibodies with antipeptide activity are tested for anti-MDDT activity using protocols well known in the art, including ELISA, RIA, and immunoblotting.

Antibody fragments containing specific binding sites for an epitope may also be generated. For example, such fragments include, but are not limited to, the F(ab′)₂ fragments produced by pepsin digestion of the antibody molecule, and the Fab fragments generated by reducing the disulfide bridges of the F(ab′)₂ fragments. Alternatively, construction of Fab expression libraries in filamentous bacteriophage allows rapid and easy identification of monoclonal fragments with desired specificity (Pound, supra, Chaps. 45-47). Antibodies generated against polypeptide encoded by mddt can be used to purify and characterize full-length MDDT protein and its activity, binding partners, etc.

Assays Using Antibodies

Anti-MDDT antibodies may be used in assays to quantify the amount of MDDT found in a particular human cell. Such assays include methods utilizing the antibody and a label to detect expression level under normal or disease conditions. The peptides and antibodies of the invention may be used with or without modification or labeled by joining them, either covalently or noncovalently, with a reporter molecule.

Protocols for detecting and measuring protein expression using either polyclonal or monoclonal antibodies are well known in the art. Examples include ELISA, RIA, and fluorescent activated cell sorting (FACS). Such immunoassays typically involve the formation of complexes between the MDDT and its specific antibody and the measurement of such complexes. These and other assays are described in Pound (supra).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications, and publications mentioned above and below, including U.S. Serial No. 60/230,517, U.S. Serial No. 60/230,599, U.S. Serial No. 60/230,514, U.S. Serial No. 60/231,167, U.S. Serial No. 60/230,598, U.S. Serial No. 60/230,988, U.S. Serial No. 60/230,518, U.S. Serial No. 60/230,515, U.S. Serial No. 60/229,751, U.S. Serial No. 60/230,610, U.S. Serial No. 60/229,749, U.S. Serial No. 60/229,750, U.S. Serial No. 60/230,597, U.S. Serial No. 60/230,505, U.S. Serial No. 60/231,163, U.S. Serial No. 60/229,747, U.S. Serial No. 60/229,748, U.S. Serial No. 60/230,583, U.S. Serial No. 60/230,519, U.S. Serial No. 60/230,595, U.S. Serial No. 60/230,865, U.S. Serial No. 60/230,989, and U.S. Serial No. 60/230,951, are hereby expressly incorporated by reference.

### EXAMPLES

I. Construction of cDNA Libraries

RNA was purchased from CLONTECH Laboratories, Inc. (Palo Alto Calif.) or isolated from various tissues. Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated with either isopropanol or sodium acetate and ethanol, or by other routine methods.

Phenol extraction and precipitation of RNA were repeated as necessary to increase RNA purity. In most cases. RNA was treated with DNase. For most libraries, poly(A+) RNA was isolated using oligo d(T)coupled [symbol] magnetic particles (Promega Corporation, Omega), Madison Wis.), OLIGOTEX latex particles (QIAGEN, Inc. (QIAGEN), Valencia Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Inc. Austin Tex.).

In some cases, Stratagene was provided with RNA and constructed the corresponding cDNA libraries. Otherwise, cDNA was synthesized and cDNA libraries were constructed with the UNIZAP vector system (Stratagene Cloning Systems, Inc. (Stratagene), La Jolla Calif.) or SUPERSCRIPT plasmid system (Life Technologies), using the recommended procedures or similar methods known in the art. (See, e.g., Ausubel, 1997, supra, Chapters 5.1 through 6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g. PBLUESCRIPT plasmid (Stratagene), PSPORT1 plasmid (Life Technologies), PCDNA2.1 plasmid (Invitrogen, Carlsbad Calif.), PBK-CMV plasmid (Stratagene), or pINCY (Incyte Genomics, Palo Alto Calif.), or derivatives thereof. Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids were recovered from host cells by in vivo excision using the UNIZAP vector system (Stratagene) or by cell lysis. Plasmids were purified using at least one of the following: the Magic or WIZARD Minipreps DNA purification system (Promega); the AGTC Miniprep purification kit (Edge BioSystems, Gaithersburg Md.); and the QIAWELL 8, QIAWELL 8 Plus, and QIAWELL 8 Ultra plasmid purification systems or the R.E.A.L. PREP 96 plasmid purification kit (QIAGEN). Following precipitation, plasmids were resuspended in 0.1 ml of distilled water and stored, with or without lyophilization, at 4° C.

Alternatively, plasmid DNA was amplified from host cell lysates using direct link PCR in a high-throughput format. (Rao, V. B. (1994) Anal. Biochem. 216:1-14.) Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Inc. (Molecular Probes), Eugene Oreg.) and a FLUOROSKAN II fluorescence scanner (Labsystems Oy, Helsinki, Finland).

III. Sequencing and Analysis

cDNA sequencing reactions were processed using standard methods or high-throughput instrumentation such as the ABI CATALYST 800 thermal cycler (Applied Biosystems) or the PTC-200 thermal cycler (MJ Research) in conjunction with the HYDRA microdispenser (Robbins Scientific Corp., Sunnyvale Calif.) or the MICROLAB 2200 liquid transfer system (Hamilton). cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, Chapter 7.7). Some of the cDNA sequences were selected for extension using the techniques disclosed in Example VIII.

IV. Assembly and Analysis of Sequences

Component sequences from chromatograms were subject to PHRED analysis and assigned a quality score. The sequences having at least a required quality score were subject to various pre-processing editing pathways to eliminate, e.g., low quality 3′ ends, vector and linker sequences, polyA tails, Alu repeats, mitochondrial and ribosomal sequences, bacterial contamination sequences, and sequences smaller than 50 base pairs. In particular, low-information sequences and repetitive elements (e.g., dinucleotide repeats, Alu repeats, etc.) were replaced by “n's”, or masked, to prevent spurious matches.

Processed sequences were then subject to assembly procedures in which the sequences were assigned to gene bins (bins). Each sequence could only belong to one bin. Sequences in each gene bin were assembled to produce consensus sequences (templates). Subsequent new sequences were added to existing bins using BLASTn (v. 1.4 WashU) and CROSSMATCH. Candidate pairs were identified as all BLAST hits having a quality score greater than or equal to 150. Alignments of at least 82% local identity were accepted into the bin. The component sequences from each bin were assembled using a version of PHRAP. Bins with several overlapping component sequences were assembled using DEEP PHRAP. The orientation (sense or antisense) of each assembled template was determined based on the number and orientation of its component sequences. Template sequences as disclosed in the sequence listing correspond to sense strand sequences (the “forward” reading frames), to the best determination. The complementary (antisense) strands are inherently disclosed herein. The component sequences which were used to assemble each template consensus sequence are listed in Table 5, along with their positions along the template nucleotide sequences.

Bins were compared against each other and those having local similarity of at least 82% were combined and reassembled. Reassembled bins having templates of insufficient overlap (less than 95% local identity) were re-split. Assembled templates were also subject to analysis by STITCHER/EXON MAPPER algorithms which analyze the probabilities of the presence of splice variants, alternatively spliced exons, splice junctions, differential expression of alternative spliced genes across tissue types or disease states, etc. These resulting bins were subject to several rounds of the above assembly procedures.

Once gene bins were generated based upon sequence alignments, bins were clone joined based upon clone information. If the 5′sequence of one clone was present in one bin and the 3′ sequence from the same clone was present in a different bin, it was likely that the two bins actually belonged together in a single bin. The resulting combined bins underwent assembly procedures to regenerate the consensus sequences.

The final assembled templates were subsequently annotated using the following procedure. Template sequences were analyzed using BLASTn (v2.0, NCBI) versus gbpri (GenBank version 124). “Hits” were defined as an exact match having from 95% local identity over 200 base pairs through 100% local identity over 100 base pairs, or a homolog match having an E-value, i.e. a probability score, of <1×10⁻⁸. The hits were subject to frameshift FASTx versus GENPEPT (GenBank version 124). (See Table 8). In this analysis, a homolog match was defined as having an E-value of <1×10⁻⁸. The assembly method used above was described in “System and Methods for Analyzing Biomolecular Sequences,” U.S. Ser. No. 09/276,534, filed Mar. 25, 1999, and the LIFESEQ Gold user manual (Incyte) both incorporated by reference herein.

Following assembly, template sequences were subjected to motif, BLAST, and functional analyses, and categorized in protein hierarchies using methods described in, e.g., “Database System Employing Protein Function Hierarchies for Viewing Biomolecular Sequence Data,” U.S. Ser. No. 08/812,290, filed Mar. 6, 1997; “Relational Database for Storing Biomolecule Information,” U.S. Ser. No. 08/947,845, filed Oct. 9, 1997; “Project-Based Full-Length Biomolecular Sequence Database,” U.S. Ser. No. 08/811,758, filed Mar. 6, 1997; and “Relational Database and System for Storing Information Relating to Biomolecular Sequences,” U.S. Ser. No. 09/034,807, filed Mar. 4, 1998, all of which are incorporated by reference herein.

The template sequences were further analyzed by translating each template in all three forward reading frames and searching each translation against the Pfam database of hidden Markov model-based protein families and domains using the HMMER software package (available to the public from Washington University School of Medicine, St. Louis Mo.). Regions of templates which, when translated, contained polarity to Pfam consensus sequences are reported in Table 3, along with descriptions of Pfam protein domains and families. Only those Pfam hits with an E-value of <1×10⁻³ are reported. (See also World Wide Web site http://pfam.wustl.edu/ for detailed descriptions of Pfam protein domains and families.)

Additionally, the template sequences were translated in all three forward reading frames, and each translation was searched against hidden Markov models for signal peptides using the HMMER software package. Construction of hidden Markov models and their usage in sequence analysis has been described. (See, for example, Eddy, S. R. (1996) Curr. Opin. Str. Biol. 6:361-365.) Only those signal peptide hits with a cutoff score of 11 bits or greater are reported. A cutoff score of 11 bits or greater corresponds to at least about 91-94% true-positives in signal peptide prediction. Template sequences were also translated in all three forward reading frames, and each translation was searched against TMAP, a program that uses weight matrices to delineate transmembrane segments on protein sequences and determine orientation, with respect to the cell cytosol (Persson, B. and P. Argos (1994) J. Mol. Biol. 237:182-192; Persson, B. and P. Argos (1996) Protein Sci. 5:363-371.) Regions of templates which, when translated, contain similarity to signal peptide or transmembrane consensus sequences are reported in Table 4.

The results of HMMER analysis as reported in Tables 3 and 4 may support the results of BLAST analysis as reported in Table 2 or may suggest alternative or additional properties of template-encoded polypeptides not previously uncovered by BLAST or other analyses.

Template sequences are further analyzed using the bioinformatics tools listed in Table 8, or using sequence analysis software known in the art such as MACDNASIS PRO software (Hitachi Software Engineering, South San Francisco Calif.) and LASERGENE software (DNASTAR). Template sequences may be further queried against public databases such as the GenBank rodent, mammalian, vertebrate, prokaryote, and eukaryote databases.

The template sequences were translated to derive the corresponding longest open reading frame as presented by the polypeptide sequences as reported in Table 2. Alternatively, a polypeptide of the invention may begin at any of the methionine residues within the full length translated polypeptide. Polypeptide sequences were subsequently analyzed by querying against the GenBank protein database (GENPEPT, (GenBank version 124)). Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 shows sequences with homology to the polypeptides of the invention as identified by BLAST analysis against a GenBank protein (GENPEPT) database. Column 1 shows the polypeptide sequence identification number (SEQ ID NO:) for the polypeptide segments of the invention. Column 2 shows the reading frame used in the translation of the polynucleotide sequences encoding the polypeptide segments. Column 3 shows the length of the translated polypeptide segments. Columns 4 and 5 show the start and stop nucleotide positions of the polynucleotide sequences encoding the polypeptide segments. Column 6 shows the GenBank identification number (GI Number) of the nearest GenBank homolog. Column 7 shows the probability score for the match between each polypeptide and its GenBank homolog. Column 8 shows the annotation of the GenBank homolog.

V. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel, 1995, supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score [symbol] is produced either by 100% identity and 50% overlap at one end, or 79% identity and 100% overlap.

VI. Tissue Distribution Profiling

A tissue distribution profile is determined for each template by compiling the cDNA library tissue classifications of its component cDNA sequences. Each component sequence, is derived from a cDNA library constructed from a human tissue. Each human tissue is classified into one of the following categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. Template sequences, component sequences, and cDNA library/tissue information are found in the LIFESEQ GOLD database (Incyte Genomics, Palo Alto Calif.).

Table 6 shows the tissue distribution profile for the templates of the invention. For each template, the three most frequently observed tissue categories are shown in column 3, along with the percentage of component sequences belonging to each category. Only tissue categories with percentage values of ≧10% are shown. A tissue distribution of “widely distributed” in column 3 indicates percentage values of <10% in all tissue categories.

VII. Transcript Image Analysis

Transcript images are generated as described in Seilhamer et al., “Comparative Gene Transcript Analysis,” U.S. Pat. No. 5,840,484, incorporated herein by reference.

VIII. Extension of Polynucleotide Sequences and Isolation of a Full-Length cDNA

Oligonucleotide primers designed using an mddt of the Sequence Listing are used to extend the nucleic acid sequence. One primer is synthesized to initiate 5′ extension of the template, and the other primer, to initiate 3′ extension of the template. The initial primers may be designed using OLIGO 4.06 software (National Biosciences, Inc. (National Biosciences), Plymouth Minn.), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations are avoided. Selected human cDNA libraries are used to extend the sequence. If more than one extension is necessary or desired, additional or nested sets of primers are designed.

High fidelity amplification is obtained by PCR using methods well known in the art. PCR is performed in 96-well plates using the PTC-200 thermal cycler (MJ Research). The reaction mix contains DNA template, [symbol] nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C. 15 sec; Step 3: 60° C., 1 min: Step 4: 68° C. 2 min; Step 5: Steps 2, 3, and 4 repeated 20 times; Step 6: 68° C., 5 min; Step 7: storage at 4° C. In the alternative, the parameters for primer pair T7 and SK+ are as follows: Step 1: 94° C. 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well is determined by dispensing 100 μl PICOGREEN quantitation reagent (0.25% (v/v); Molecular Probes) dissolved in 1× Tris-EDTA (TE) and 0.5 μl of undiluted PCR product into each well of an opaque fluorimeter plate (Corning Incorporated (Corning), Corning N.Y.), allowing the DNA to bind to the reagent. The plate is scanned in a FLUOROSKAN II (Labsystems Oy) to measure the fluorescence of the sample and to quantify the concentration of DNA. A 5 μl to 10 μl aliquot of the reaction mixture is analyzed by electrophoresis on a 1% agarose mini-gel to determine which reactions are successful in extending the sequence.

The extended nucleotides are desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides are separated on low concentration (0.6 to 0.8%) agarose gels, fragments are excised, and agar digested with AGAR ACE (Promega). Extended clones are religated using T4 ligase (New England Biolabs, Inc., Beverly Mass.) into pUC 18 vector (Amersham Pharmacia Biotech), treated with Pfu DNA polymerase (Stratagene) to fill-in restriction site overhangs, and transfected into competent E. coli cells. Transformed cells are selected on antibiotic-containing media, individual colonies are picked and cultured overnight at 37° C. in 384-well plates in LB/2× carbenicillin liquid media.

The cells are lysed, and DNA is amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA is quantified by PICOGREEN reagent (Molecular Probes) as described above. Samples with low DNA recoveries are reamplified using the same conditions as described above. Samples are diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Applied Biosystems).

In like manner, the mddt is used to obtain regulatory sequences (promoters, introns, and enhancers) using the procedure above, oligonucleotides designed for su [symbol] extension, and an appropriate genomic library.

IX. Labeling of Probes and Southern Hybridization Analyses

Hybridization probes derived from the mddt of the Sequence Listing are employed for screening cDNAs, mRNAs, or genomic DNA. The labeling of probe nucleotides between 100 and 1000 nucleotides in length is specifically described, but essentially the same procedure may be used with larger cDNA fragments. Probe sequences are labeled at room temperature for 30 minutes using a T4 polynucleotide kinase, γ³²P-ATP, and 0.5×One-Phor-AII Plus (Amersham Pharmacia Biotech) buffer and purified using a ProbeQuant G-50 Microcolumn (Amersham Pharmacia Biotech). The probe mixture is diluted to 10⁷ dpm/μg/ml hybridization buffer and used in a typical membrane-based hybridization analysis.

The DNA is digested with a restriction endonuclease such as Eco RV and is electrophoresed through a 0.7% agarose gel. The DNA fragments are transferred from the agarose to nylon membrane (NYTRAN Plus, Schleicher & Schuell, Inc., Keene N.H.) using procedures specified by the manufacturer of the membrane. Prehybridization is carried out for three or more hours at 68° C., and hybridization is carried out overnight at 68° C. To remove non-specific signals, blots are sequentially washed at room temperature under increasingly stringent conditions, up to 0.1× saline sodium citrate (SSC) and 0.5% sodium dodecyl sulfate. After the blots are placed in a PHOSPHORIMAGER cassette (Molecular Dynamics) or are exposed to autoradiography film, hybridization patterns of standard and experimental lanes are compared. Essentially the same procedure is employed when screening RNA.

X. Chromosome Mapping of mddt

The cDNA sequences which were used to assemble SEQ ID NO:1-252 are compared with sequences from the Incyte LIFESEQ database and public domain databases using BLAST and other implementations of the Smith-Waterman algorithm. Sequences from these databases that match SEQ ID NO:1-252 are assembled into clusters of contiguous and overlapping sequences using assembly algorithms such as PHRAP (Table 8). Radiation hybrid and genetic mapping data available from public resources such as the Stanford Human Genome Center (SHGC), Whitehead Institute for Genome Research (WIGR), and Généthon are used to determine if any of the clustered sequences have been previously mapped. Inclusion of a mapped sequence in a cluster will result in the assignment of all sequences of that cluster, including its particular SEQ ID NO:, to that map location. The genetic map locations of SEQ ID NO:1-252 are described as ranges, or intervals, of human chromosomes. The map position of an interval, in centiMorgans, is measured relative to the terminus of the chromosome's p- [symbol] . The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters.

XI. Microarray Analysis

Probe Preparation from Tissue or Cell Samples

Total RNA is isolated from tissue samples using the guanidinium thiocyanate method and polyA⁺ RNA is purified using the oligo (dT) cellulose method. Each polyA⁺ RNA sample is reverse transcribed using MMLV reverse-transcriptase, 0.05 pg/μl oligo-dT primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Pharmacia Biotech). The reverse transcription reaction is performed in a 25 ml volume containing 200 ng polyA⁺ RNA with GEMBRIGHT kits (Incyte). Specific control polyA⁺ RNAs are synthesized by in vitro transcription from non-coding yeast genomic DNA (W. Lei, unpublished). As quantitative controls, the control mRNAs at 0.002 ng, 0.02 ng, 0.2 ng, and 2 ng are diluted into reverse transcription reaction at ratios of 1:100,000, 1:10,000, 1:1000, 1:100 (w/w) to sample mRNA respectively. The control mRNAs are diluted into reverse transcription reaction at ratios of 1:3, 3:1, 1:10, 10:1, 1:25, 25:1 (w/w) to sample mRNA differential expression patterns. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Probes are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The probe is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester, Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford, Mass.) for 30 minutes at 60° C. followed by washes in 0.2% SDS and distilled water as before.

Hybridization

Hybridization reactions contain 9 μl of probe mixture consisting of 0.2 μg each of Cy3 and Cy5 labeled cDNA synthesis products in 5×SSC, 0.2% SDS hybridization buffer. The probe mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the probe mix at a known concentration. A specific location on the array contains a complementary DNA sequence, allowing the intensity of the signal at that location to be correlated with a weight ratio of hybridizing species of 1:100,000. When two probes from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood, Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte).

XII. Complementary Nucleic Acids

Sequences complementary to the mddt are used to detect, decrease, or inhibit expression of the naturally occurring nucleotide. The use of oligonucleotides comprising from about 15 to 30 base pairs is typical in the art. However, smaller or larger sequence fragments can also be used. Appropriate oligonucleotides are designed from the mddt using OLIGO 4.06 software (National Biosciences) or other appropriate programs and are synthesized using methods standard in the art or ordered from a commercial supplier. To inhibit transcription, a complementary oligonucleotide is designed from the most unique 5′ sequence and used to prevent transcription factor binding to the promoter sequence. To inhibit translation, a complementary oligonucleotide is designed to prevent ribosomal binding and processing of the transcript.

XIII. Expression of MDDT

Expression and purification of MDDT is accomplished using bacterial or virus-based expression systems. For expression of MDDT in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts. e.g., BL21(DE3). Antibiotic resistant bacteria express MDDT upon induction with isopropyl beta-D-thiogalactopyranoside (IPTG). Expression of MDDT in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding MDDT by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See e.g., Engelhard, supra, and Sandig, supra.)

In most expression systems, MDDT is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Pharmacia Biotech). Following purification, the GST moiety can be proteolytically cleaved from MDDT at specifically engineered sites. FLAG, an 8-amino acid peptide, enables immunoaffinity purification using commercially available monoclonal and polyclonal anti-FLAG antibodies (Eastman Kodak Company, Rochester N.Y.). 6-His, a stretch of six consecutive histidine residues, enables purification on metal-chelate resins (QIAGEN). Methods for protein expression and purification are discussed in Ausubel (1995, supra, Chapters 10 and 16). Purified MDDT obtained by these methods can be used directly in the following activity assay.

XIV. Demonstration of MDDT Activity

MDDT, or biologically active fragments thereof, are labeled with ²⁵¹I Bolton-Hunter reagent. (See, e.g., Bolton, A. E. and W. M. Hunter (1973) Biochem. J. 133:529-539.) Candidate molecules previously arrayed in the wells of a multi-well plate are incubated with the labeled MDDT, washed, and any wells with labeled MDDT complex are assayed. Data obtained using different concentrations of MDDT are used to calculate values for the number, affinity, and association of MDDT with the candidate molecules.

Alternatively, molecules interacting with MDDT are analyzed using the yeast two-hybrid system as described in Fields, S. and O. Song (1989) Nature 340:245-246, or using commercially available kits based on the two-hybrid system, such as the MATCHMAKER system (CLONTECH).

MDDT may also be used in the PATHCALLING process (CuraGen Corp. New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XV. Functional Assays

MDDT function is assessed by expressing mddt at physiologically elevated levels in mammalian cell culture systems. cDNA is subcloned into a mammalian expression vector containing a strong promoter that drives high levels of cDNA expression. Vectors of choice include pCMV SPORT (Life Technologies) and pCR3.1 (Invitrogen Corporation, Carlsbad Calif.), both of which contain the cytomegalovirus promoter. 5-10 μg of recombinant vector are transiently transfected into a human cell line, preferably of endothelial or hematopoietic origin, using either liposome formulations or electroporation. 1-2 μg of an additional plasmid containing sequences encoding a marker protein are co-transfected.

Expression of a marker protein provides a means to distinguish transfected cells from nontransfected cells and is a reliable predictor of cDNA expression from the recombinant vector. Marker proteins of choice include, e.g., Green Fluorescent Protein (GFP; CLONTECH), CD64, or a CD64-GFP fusion protein. Flow cytometry (FCM), an automated laser optics-based technique, is used to identify transfected cells expressing GFP or CD64-GFP and to evaluate the apoptotic state of the cells and other cellular properties.

FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of MDDT on gene expression can be assessed using highly purified populations of cells transfected with sequences encoding MDDT and either CD64 or CD64-GFP. CD64 and CD64-GFP are expressed as the surface of transfected cells and bind to observed regions of human immunoglobulin G (IgG). Transfected cells are efficiently separated from nontransfected cells using magnetic beads coated with either human IgG or antibody against CD64 (DYNAL, Inc., Lake Success N.Y.). mRNA can be purified from the cells using methods well known by those of skill in the art. Expression of mRNA encoding MDDT and other genes of interest can be analyzed by northern analysis or microarray techniques.

XVI. Production of Antibodies

MDDT substantially purified using polyacrylamide gel electrophoresis (PAGE; see, e.g., Harrington, M. G. (1990) Methods Enzymol. 182:488-495), or other purification techniques, is used to immunize rabbits and to produce antibodies using standard protocols.

Alternatively, the MDDT amino acid sequence is analyzed using LASERGENE software (DNASTAR) to determine regions of high immunogenicity, and a corresponding peptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, Chapter 11.)

Typically, peptides 15 residues in length are synthesized using an ABI 431A peptide synthesizer (Applied Biosystems) using fmoc-chemistry and coupled to KLH (Sigma) by reaction with N-maleimidobenzoyl-N-hydroxysuccinimide ester (MBS) to increase immunogenicity. (See, e.g., Ausubel, supra.) Rabbits are immunized with the peptide-KLH complex in complete Freund's adjuvant. Resulting antisera are tested for antipeptide activity by, for example, binding the peptide to plastic, blocking with 1% BSA, reacting with rabbit antisera, washing, and reacting with radio-iodinated goat anti-rabbit IgG. Antisera with antipeptide activity are tested for anti-MDDT activity using protocols well known in the art, including ELISA, RIA, and immunoblotting.

XVII. Purification of Naturally Occurring MDDT Using Specific Antibodies

Naturally occurring or recombinant MDDT is substantially purified by immunoaffinity chromatography using antibodies specific for MDDT. An immunoaffinity column is constructed by covalently coupling anti-MDDT antibody to an activated chromatographic resin, such as CNBr-activated SEPHAROSE (Amersham Pharmacia Biotech). After the coupling, the resin is blocked and washed according to the manufacturer's instructions.

Media containing MDDT are passed over the immunoaffinity column, and the column is washed under conditions that allow the preferential absorbance of MDDT (e.g., high ionic strength buffers in the presence of detergent). The column is eluted under conditions that disrupt antibody/MDDT binding (e.g., a buffer of pH 2 to pH 3, or a high concentration of a chaotrope, such as urea or thiocyanate io [symbol] nd MDDT is collected.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

TABLE 1

| SEQ ID NO: | Template ID | SEQ ID NO: | ORF ID |
|---|---|---|---|
| 1 | LG:150318.1:2000SEP08 | 253 | LG:150318.1.orf2:2000SEP08 |
| 2 | LG:022529.1:2000SEP08 | 254 | LG:022529.1.orf1:2000SEP08 |
| 3 | LG:352559.1:2000SEP08 | 255 | LG:352559.1.orf2:2000SEP08 |
| 4 | LG:175223.1:2000SEP08 | 256 | LG:175223.1.orf3:2000SEP08 |
| 5 | LG:476989.1:2000SEP08 | 257 | LG:476989.1.orf2:2000SEP08 |
| 6 | LG:253268.7:2000SEP08 | 258 | LG:253268.7.orf1:2000SEP08 |
| 7 | LG:401322.1:2000SEP08 | 259 | LG:401322.1.orf1:2000SEP08 |
| 8 | LG:1328436.1:2000SEP08 | 260 | LG:1328436.1.orf2:2000SEP08 |
| 9 | LG:475404.1:2000SEP08 | 261 | LG:475404.1.orf2:2000SEP08 |
| 10 | LG:1384132.1:2000SEP08 | 262 | LG:1384132.1.orf1:2000SEP08 |
| 11 | LG:410804.18:2000SEP08 | 263 | LG:410804.18.orf1:2000SEP08 |
| 12 | LG:1082306.1:2000SEP08 | 264 | LG:1082306.1.orf1:2000SEP08 |
| 13 | LG:233814.4:2000SEP08 | 265 | LG:233814.4.orf1:2000SEP08 |
| 14 | LG:977478.5:2000SEP08 | 266 | LG:977478.5.orf3:2000SEP08 |
| 15 | LG:025931.1:2000SEP08 | 267 | LG:025931.1.orf2:2000SEP08 |
| 16 | LG:885368.1:2000SEP08 | 268 | LG:885368.1.orf1:2000SEP08 |
| 17 | LG:1054900.1:2000SEP08 | 269 | LG:1054900.1.orf3:2000SEP08 |
| 18 | LG:995186.2:2000SEP08 | 270 | LG:995186.2.orf3:2000SEP08 |
| 19 | LG:435048.23:2000SEP08 | 271 | LG:435048.23.orf2:2000SEP08 |
| 20 | LG:954859.1:2000SEP08 | 272 | LG:954859.1.orf2:2000SEP08 |
| 21 | LG:364370.1:2000SEP08 | 273 | LG:364370.1.orf3:2000SEP08 |
| 22 | LG:1098789.1:2000SEP08 | 274 | LG:1098789.1.orf3:2000SEP08 |
| 23 | LG:201540.2:2000SEP08 | 275 | LG:201540.2.orf2:2000SEP08 |
| 24 | LG:1077357.1:2000SEP08 | 276 | LG:1077357.1.orf1:2000SEP08 |
| 25 | LG:1048846.4:2000SEP08 | 277 | LG:1048846.4.orf3:2000SEP08 |
| 26 | LG:336685.1:2000SEP08 | 278 | LG:336685.1.orf3:2000SEP08 |
| 27 | LG:1076253.1:2000SEP08 | 279 | LG:1076253.1.orf3:2000SEP08 |
| 28 | LG:1400601.2:2000SEP08 | 280 | LG:1400601.2.orf3:2000SEP08 |
| 29 | LG:1079092.3:2000SEP08 | 281 | LG:1079092.3.orf2:2000SEP08 |
| 30 | LG:1086064.1:2000SEP08 | 282 | LG:1086064.1.orf2:2000SEP08 |
| 31 | LG:1400608.1:2000SEP08 | 283 | LG:1400608.1.orf3:2000SEP08 |
| 32 | LG:399275.5:2000SEP08 | 284 | LG:399275.5.orf1:2000SEP08 |
| 33 | LG:293943.1:2000SEP08 | 285 | LG:293943.1.orf1:2000SEP08 |
| 34 | LG:345884.1:2000SEP08 | 286 | LG:345884.1.orf2:2000SEP08 |
| 35 | LG:400967.1:2000SEP08 | 287 | LG:400967.1.orf1:2000SEP08 |
| 36 | LG:024556.6:2000SEP08 | 288 | LG:024556.6.orf1:2000SEP08 |
| 37 | LG:081189.3:2000SEP08 | 289 | LG:081189.3.orf2:2000SEP08 |
| 38 | LG:018258.1:2000SEP08 | 290 | LG:018258.1.orf3:2000SEP08 |
| 39 | LG:450399.3:2000SEP08 | 291 | LG:450399.3.orf3:2000SEP08 |
| 40 | LG:451122.1:2000SEP08 | 292 | LG:451122.1.orf1:2000SEP08 |
| 41 | LG:451682.1:2000SEP08 | 293 | LG:451682.1.orf3:2000SEP08 |
| 42 | LG:238631.4:2000SEP08 | 294 | LG:238631.4.orf3:2000SEP08 |
| 43 | LG:236654.1:2000SEP08 | 295 | LG:236654.1.orf3:2000SEP08 |
| 44 | LG:332655.1:2000SEP08 | 296 | LG:332655.1.orf1:2000SEP08 |
| 45 | LG:217396.2:2000SEP08 | 297 | LG:217396.2.orf1:2000SEP08 |
| 46 | LG:090574.1:2000SEP08 | 298 | LG:090574.1.orf3:2000SEP08 |
| 47 | LG:202943.1:2000SEP08 | 299 | LG:202943.1.orf1:2000SEP08 |
| 48 | LG:236928.1:2000SEP08 | 300 | LG:236928.1.orf1:2000SEP08 |
| 49 | LG:215169.2:2000SEP08 | 301 | LG:215169.2.orf2:2000SEP08 |
| 50 | LG:410726.1:2000SEP08 | 302 | LG:410726.1.orf1:2000SEP08 |
| 51 | LG:234372.2:2000SEP08 | 303 | LG:234372.2.orf2:2000SEP08 |
| 52 | LG:022629.1:2000SEP08 | 304 | LG:022629.1.orf3:2000SEP08 |
| 53 | LG:068682.1:2000SEP08 | 305 | LG:068682.1.orf2:2000SEP08 |
| 54 | LG:222335.1:2000SEP08 | 306 | LG:222335.1.orf1:2000SEP08 |
| 55 | LG:331342.1:2000SEP08 | 307 | LG:331342.1.orf3:2000SEP08 |
| 56 | LG:021770.1:2000SEP08 | 308 | LG:021770.1.orf3:2000SEP08 |
| 57 | LG:181607.9:2000SEP08 | 309 | LG:181607.9.orf2:2000SEP08 |
| 58 | LG:1042768.1:2000SEP08 | 310 | LG:1042768.1.orf3:2000SEP08 |
| 59 | LG:282729.1:2000SEP08 | 311 | LG:282729.1.orf1:2000SEP08 |
| 60 | LG:998305.3:2000SEP08 | 312 | LG:998305.3.orf2:2000SEP08 |
| 61 | LG:1135213.1:2000SEP08 | 313 | LG:1135213.1.orf1:2000SEP08 |
| 62 | LG:267762.1:2000SEP08 | 314 | LG:267762.1.orf1:2000SEP08 |
| 63 | LG:120744.1:2000SEP08 | 315 | LG:120744.1.orf1:2000SEP08 |
| 64 | LG:403409.1:2000SEP08 | 316 | LG:403409.1.orf3:2000SEP08 |
| 65 | LG:226874.3:2000SEP08 | 317 | LG:226874.3.orf1:2000SEP08 |
| 66 | LG:1045521.4:2000SEP08 | 318 | LG:1045521.4.orf1:2000SEP08 |
| 67 | LG:275876.1:2000SEP08 | 319 | LG:275876.1.orf1:2000SEP08 |
| 68 | LG:475127.7:2000SEP08 | 320 | LG:475127.7.orf1:2000SEP08 |
| 69 | LG:157263.1:2000SEP08 | 321 | LG:157263.1.orf1:2000SEP08 |
| 70 | LG:247382.7:2000SEP08 | 322 | LG:247382.7.orf1:2000SEP08 |
| 71 | LG:197367.5:2000SEP08 | 323 | LG:197367.5.orf2:2000SEP08 |
| 72 | LG:218090.5:2000SEP08 | 324 | LG:218090.5.orf2:2000SEP08 |
| 73 | LG:216612.4:2000SEP08 | 325 | LG:216612.4.orf3:2000SEP08 |
| 74 | LG:197614.1:2000SEP08 | 326 | LG:197614.1.orf1:2000SEP08 |
| 75 | LG:378428.1:2000SEP08 | 327 | LG:378428.1.orf3:2000SEP08 |
| 76 | LG:286639.1:2000SEP08 | 328 | LG:286639.1.orf3:2000SEP08 |
| 77 | LG:389870.1:2000SEP08 | 329 | LG:389870.1.orf1:2000SEP08 |
| 78 | LG:1387485.6:2000SEP08 | 330 | LG:1387485.6.orf2:2000SEP08 |
| 79 | LG:230151.1:2000SEP08 | 331 | LG:230151.1.orf1:2000SEP08 |
| 80 | LG:215158.5:2000SEP08 | 332 | LG:215158.5.orf1:2000SEP08 |
| 81 | LG:235840.1:2000SEP08 | 333 | LG:235840.1.orf2:2000SEP08 |
| 82 | LG:350272.1:2000SEP08 | 334 | LG:350272.1.orf2:2000SEP08 |
| 83 | LG:232190.1:2000SEP08 | 335 | LG:232190.1.orf2:2000SEP08 |
| 84 | LG:1068127.1:2000SEP08 | 336 | LG:1068127.1.orf2:2000SEP08 |
| 85 | LG:408751.3:2000SEP08 | 337 | LG:408751.3.orf2:2000SEP08 |
| 86 | LG:1078933.1:2000SEP08 | 338 | LG:1078933.1.orf1:2000SEP08 |
| 87 | LG:958731.1:2000SEP08 | 339 | LG:958731.1.orf3:2000SEP08 |
| 88 | LG:024125.5:2000SEP08 | 340 | LG:024125.5.orf3:2000SEP08 |
| 89 | LG:373637.3:2000SEP08 | 341 | LG:373637.3.orf1:2000SEP08 |
| 90 | LG:1053229.1:2000SEP08 | 342 | LG:1053229.1.orf1:2000SEP08 |
| 91 | LG:248364.1:2000SEP08 | 343 | LG:248364.1.orf2:2000SEP08 |
| 92 | LG:477130.1:2000SEP08 | 344 | LG:477130.1.orf3:2000SEP08 |
| 93 | LG:113786.17:2000SEP08 | 345 | LG:113786.17.orf2:2000SEP08 |
| 94 | LG:347635.1:2000SEP08 | 346 | LG:347635.1.orf3:2000SEP08 |
| 95 | LG:242966.4:2000SEP08 | 347 | LG:242966.4.orf1:2000SEP08 |
| 96 | LG:217814.1:2000SEP08 | 348 | LG:217814.1.orf3:2000SEP08 |
| 97 | LG:476452.1:2000SEP08 | 349 | LG:476452.1.orf3:2000SEP08 |
| 98 | LG:1100657.1:2000SEP08 | 350 | LG:1100657.1.orf1:2000SEP08 |
| 99 | LG:1132418.2:2000SEP08 | 351 | LG:1132418.2.orf1:2000SEP08 |
| 100 | LG:1098570.1:2000SEP08 | 352 | LG:1098570.1.orf3:2000SEP08 |
| 101 | LG:1097987.1:2000SEP08 | 353 | LG:1097987.1.orf3:2000SEP08 |
| 102 | LG:337818.2:2000SEP08 | 354 | LG:337818.2.orf1:2000SEP08 |
| 103 | LG:1040582.1:2000SEP08 | 355 | LG:1040582.1.orf2:2000SEP08 |
| 104 | LG:1099122.1:2000SEP08 | 356 | LG:1099122.1.orf3:2000SEP08 |
| 105 | LG:1327449.1:2000SEP08 | 357 | LG:1327449.1.orf3:2000SEP08 |
| 106 | LG:227933.5:2000SEP08 | 358 | LG:227933.5.orf1:2000SEP08 |
| 107 | LG:1043709.2:2000SEP08 | 359 | LG:1043709.2.orf3:2000SEP08 |
| 108 | LG:1099871.1:2000SEP08 | 360 | LG:1099871.1.orf1:2000SEP08 |
| 109 | LG:1399139.4:2000SEP08 | 361 | LG:1399139.4.orf3:2000SEP08 |
| 110 | LG:236386.1:2000SEP08 | 362 | LG:236386.1.orf3:2000SEP08 |
| 111 | LG:1015157.1:2000SEP08 | 363 | LG:1015157.1.orf1:2000SEP08 |
| 112 | LG:1065433.1:2000SEP08 | 364 | LG:1065433.1.orf3:2000SEP08 |
| 113 | LG:236992.4:2000SEP08 | 365 | LG:236992.4.orf1:2000SEP08 |
| 114 | LG:1071124.1:2000SEP08 | 366 | LG:1071124.1.orf1:2000SEP08 |
| 115 | LG:206425.2:2000SEP08 | 367 | LG:206425.2.orf2:2000SEP08 |
| 116 | LG:885747.2:2000SEP08 | 368 | LG:885747.2.orf3:2000SEP08 |
| 117 | LG:1140501.1:2000SEP08 | 369 | LG:1140501.1.orf1:2000SEP08 |
| 118 | LG:001239.1:2000SEP08 | 370 | LG:001239.1.orf3:2000SEP08 |
| 119 | LG:018980.1:2000SEP08 | 371 | LG:018980.1.orf1:2000SEP08 |
| 120 | LG:1083120.3:2000SEP08 | 372 | LG:1083120.3.orf3:2000SEP08 |
| 121 | LG:233258.3:2000SEP08 | 373 | LG:233258.3.orf3:2000SEP08 |
| 122 | LG:999062.1:2000SEP08 | 374 | LG:999062.1.orf3:2000SEP08 |
| 123 | LG:887776.1:2000SEP08 | 375 | LG:887776.1.orf3:2000SEP08 |
| 124 | LG:1400301.2:2000SEP08 | 376 | LG:1400301.2.orf1:2000SEP08 |
| 125 | LG:1329362.1:2000SEP08 | 377 | LG:1329362.1.orf2:2000SEP08 |
| 126 | LG:1096498.1:2000SEP08 | 378 | LG:1096498.1.orf2:2000SEP08 |
| 127 | LG:1096337.1:2000SEP08 | 379 | LG:1096337.1.orf2:2000SEP08 |
| 128 | LG:1400579.1:2000SEP08 | 380 | LG:1400579.1.orf2:2000SEP08 |
| 129 | LG:1080091.1:2000SEP08 | 381 | LG:1080091.1.orf2:2000SEP08 |
| 129 | LG:1080091.1:2000SEP08 | 382 | LG:1080091.1.orf3:2000SEP08 |
| 130 | LG:1082203.1:2000SEP08 | 383 | LG:1082203.1.orf1:2000SEP08 |
| 131 | LG:1084051.1:2000SEP08 | 384 | LG:1084051.1.orf3:2000SEP08 |
| 132 | LG:1082393.1:2000SEP08 | 385 | LG:1082393.1.orf3:2000SEP08 |
| 133 | LG:1086183.1:2000SEP08 | 386 | LG:1086183.1.orf2:2000SEP08 |
| 134 | LG:1090268.1:2000SEP08 | 387 | LG:1090268.1.orf3:2000SEP08 |
| 135 | LG:1400597.5:2000SEP08 | 388 | LG:1400597.5.orf1:2000SEP08 |
| 136 | LG:1080307.2:2000SEP08 | 389 | LG:1080307.2.orf3:2000SEP08 |
| 137 | LG:1400603.2:2000SEP08 | 390 | LG:1400603.2.orf2:2000SEP08 |
| 138 | LG:1052984.1:2000SEP08 | 391 | LG:1052984.1.orf1:2000SEP08 |
| 139 | LG:1091259.1:2000SEP08 | 392 | LG:1091259.1.orf3:2000SEP08 |
| 140 | LG:1082263.2:2000SEP08 | 393 | LG:1082263.2.orf3:2000SEP08 |
| 141 | LG:1048604.2:2000SEP08 | 394 | LG:1048604.2.orf3:2000SEP08 |
| 142 | LG:1085254.3:2000SEP08 | 395 | LG:1085254.3.orf2:2000SEP08 |
| 143 | LG:1400606.2:2000SEP08 | 396 | LG:1400606.2.orf1:2000SEP08 |
| 144 | LG:1090358.2:2000SEP08 | 397 | LG:1090358.2.orf1:2000SEP08 |
| 145 | LG:1079064.2:2000SEP08 | 398 | LG:1079064.2.orf1:2000SEP08 |
| 146 | LG:1076866.1:2000SEP08 | 399 | LG:1076866.1.orf2:2000SEP08 |
| 147 | LG:969359.1:2000SEP08 | 400 | LG:969359.1.orf2:2000SEP08 |
| 148 | LG:366783.1:2000SEP08 | 401 | LG:366783.1.orf1:2000SEP08 |
| 149 | LG:332176.3:2000SEP08 | 402 | LG:332176.3.orf2:2000SEP08 |
| 150 | LG:994938.1:2000SEP08 | 403 | LG:994938.1.orf3:2000SEP08 |
| 151 | LG:982800.1:2000SEP08 | 404 | LG:982800.1.orf1:2000SEP08 |
| 152 | LG:977850.7:2000SEP08 | 405 | LG:977850.7.orf1:2000SEP08 |
| 153 | LG:234748.2:2000SEP08 | 406 | LG:234748.2.orf3:2000SEP08 |
| 154 | LG:306284.1:2000SEP08 | 407 | LG:306284.1.orf2:2000SEP08 |
| 155 | LI:333170.3:2000SEP08 | 408 | LI:333170.3.orf1:2000SEP08 |
| 156 | LI:336685.2:2000SEP08 | 409 | LI:336685.2.orf3:2000SEP08 |
| 157 | LI:279013.5:2000SEP08 | 410 | LI:279013.5.orf3:2000SEP08 |
| 158 | LI:1037075.1:2000SEP08 | 411 | LI:1037075.1.orf1:2000SEP08 |
| 159 | LI:1073403.1:2000SEP08 | 412 | LI:1073403.1.orf2:2000SEP08 |
| 160 | LI:1075296.1:2000SEP08 | 413 | LI:1075296.1.orf1:2000SEP08 |
| 161 | LI:1085501.1:2000SEP08 | 414 | LI:1085501.1.orf3:2000SEP08 |
| 162 | LI:1086181.1:2000SEP08 | 415 | LI:1086181.1.orf2:2000SEP08 |
| 163 | LI:1164493.1:2000SEP08 | 416 | LI:1164493.1.orf1:2000SEP08 |
| 164 | LI:1175097.1:2000SEP08 | 417 | LI:1175097.1.orf1:2000SEP08 |
| 165 | LI:1092948.1:2000SEP08 | 418 | LI:1092948.1.orf2:2000SEP08 |
| 166 | LI:380378.2:2000SEP08 | 419 | LI:380378.2.orf3:2000SEP08 |
| 167 | LI:1029674.1:2000SEP08 | 420 | LI:1029674.1.orf3:2000SEP08 |
| 168 | LI:2048601.3:2000SEP08 | 421 | LI:2048601.3.orf2:2000SEP08 |
| 169 | LI:1186208.1:2000SEP08 | 422 | LI:1186208.1.orf2:2000SEP08 |
| 170 | LI:1170753.1:2000SEP08 | 423 | LI:1170753.1.orf1:2000SEP08 |
| 171 | LI:1180908.1:2000SEP08 | 424 | LI:1180908.1.orf2:2000SEP08 |
| 172 | LI:1182900.2:2000SEP08 | 425 | LI:1182900.2.orf1:2000SEP08 |
| 173 | LI:1169548.2:2000SEP08 | 426 | LI:1169548.2.orf3:2000SEP08 |
| 174 | LI:1039974.1:2000SEP08 | 427 | LI:1039974.1.orf1:2000SEP08 |
| 175 | LI:1175765.2:2000SEP08 | 428 | LI:1175765.2.orf3:2000SEP08 |
| 176 | LI:313948.1:2000SEP08 | 429 | LI:313948.1.orf3:2000SEP08 |
| 177 | LI:335923.2:2000SEP08 | 430 | LI:335923.2.orf2:2000SEP08 |
| 178 | LI:345884.1:2000SEP08 | 431 | LI:345884.1.orf1:2000SEP08 |
| 179 | LI:417127.1:2000SEP08 | 432 | LI:417127.1.orf1:2000SEP08 |
| 180 | LI:451710.1:2000SEP08 | 433 | LI:451710.1.orf1:2000SEP08 |
| 181 | LI:406882.2:2000SEP08 | 434 | LI:406882.2.orf1:2000SEP08 |
| 182 | LI:728223.1:2000SEP08 | 435 | LI:728223.1.orf2:2000SEP08 |
| 183 | LI:289783.19:2000SEP08 | 436 | LI:289783.19.orf2:2000SEP08 |
| 184 | LI:235255.8:2000SEP08 | 437 | LI:235255.8.orf2:2000SEP08 |
| 185 | LI:237693.5:2000SEP08 | 438 | LI:237693.5.orf3:2000SEP08 |
| 186 | LI:433670.3:2000SEP08 | 439 | LI:433670.3.orf2:2000SEP08 |
| 187 | LI:202943.4:2000SEP08 | 440 | LI:202943.4.orf2:2000SEP08 |
| 188 | LI:068682.1:2000SEP08 | 441 | LI:068682.1.orf1:2000SEP08 |
| 189 | LI:203301.3:2000SEP08 | 442 | LI:203301.3.orf2:2000SEP08 |
| 190 | LI:020726.3:2000SEP08 | 443 | LI:020726.3.orf2:2000SEP08 |
| 191 | LI:027209.1:2000SEP08 | 444 | LI:027209.1.orf1:2000SEP08 |
| 192 | LI:108819.1:2000SEP08 | 445 | LI:108819.1.orf1:2000SEP08 |
| 193 | LI:021759.1:2000SEP08 | 446 | LI:021759.1.orf2:2000SEP08 |
| 194 | LI:1165967.1:2000SEP08 | 447 | LI:1165967.1.orf1:2000SEP08 |
| 195 | LI:1166315.1:2000SEP08 | 448 | LI:1166315.1.orf3:2000SEP08 |
| 196 | LI:204626.1:2000SEP08 | 449 | LI:204626.1.orf1:2000SEP08 |
| 197 | LI:801140.1:2000SEP08 | 450 | LI:801140.1.orf1:2000SEP08 |
| 198 | LI:286639.1:2000SEP08 | 451 | LI:286639.1.orf1:2000SEP08 |
| 199 | LI:288905.4:2000SEP08 | 452 | LI:288905.4.orf3:2000SEP08 |
| 200 | LI:332161.1:2000SEP08 | 453 | LI:332161.1.orf3:2000SEP08 |
| 201 | LI:184867.1:2000SEP08 | 454 | LI:184867.1.orf1:2000SEP08 |
| 202 | LI:229932.4:2000SEP08 | 455 | LI:229932.4.orf1:2000SEP08 |
| 203 | LI:1189932.1:2000SEP08 | 456 | LI:1189932.1.orf1:2000SEP08 |
| 204 | LI:1076689.1:2000SEP08 | 457 | LI:1076689.1.orf1:2000SEP08 |
| 205 | LI:415181.2:2000SEP08 | 458 | LI:415181.2.orf1:2000SEP08 |
| 206 | LI:296358.1:2000SEP08 | 459 | LI:296358.1.orf3:2000SEP08 |
| 207 | LI:205186.3:2000SEP08 | 460 | LI:205186.3.orf2:2000SEP08 |
| 208 | LI:220537.2:2000SEP08 | 461 | LI:220537.2.orf3:2000SEP08 |
| 209 | LI:248364.2:2000SEP08 | 462 | LI:248364.2.orf2:2000SEP08 |
| 210 | LI:2048338.1:2000SEP08 | 463 | LI:2048338.1.orf3:2000SEP08 |
| 211 | LI:1185203.8:2000SEP08 | 464 | LI:1185203.8.orf3:2000SEP08 |
| 212 | LI:021770.3:2000SEP08 | 465 | LI:021770.3.orf2:2000SEP08 |
| 213 | LI:1185841.1:2000SEP08 | 466 | LI:1185841.1.orf2:2000SEP08 |
| 214 | LI:1181710.1:2000SEP08 | 467 | LI:1181710.1.orf1:2000SEP08 |
| 215 | LI:2048959.1:2000SEP08 | 468 | LI:2048959.1.orf3:2000SEP08 |
| 216 | LI:798494.1:2000SEP08 | 469 | LI:798494.1.orf3:2000SEP08 |
| 217 | LI:2049223.1:2000SEP08 | 470 | LI:2049223.1.orf1:2000SEP08 |
| 218 | LI:1177833.1:2000SEP08 | 471 | LI:1177833.1.orf3:2000SEP08 |
| 219 | LI:2049267.1:2000SEP08 | 472 | LI:2049267.1.orf3:2000SEP08 |
| 220 | LI:1165939.1:2000SEP08 | 473 | LI:1165939.1.orf1:2000SEP08 |
| 221 | LI:1170958.1:2000SEP08 | 474 | LI:1170958.1.orf1:2000SEP08 |
| 222 | LI:1089827.1:2000SEP08 | 475 | LI:1089827.1.orf2:2000SEP08 |
| 223 | LI:792112.1:2000SEP08 | 476 | LI:792112.1.orf2:2000SEP08 |
| 224 | LI:282219.2:2000SEP08 | 477 | LI:282219.2.orf3:2000SEP08 |
| 225 | LI:1088010.2:2000SEP08 | 478 | LI:1088010.2.orf3:2000SEP08 |
| 226 | LI:1165276.1:2000SEP08 | 479 | LI:1165276.1.orf3:2000SEP08 |
| 227 | LI:1169524.2:2000SEP08 | 480 | LI:1169524.2.orf1:2000SEP08 |
| 228 | LI:1180255.1:2000SEP08 | 481 | LI:1180255.1.orf1:2000SEP08 |
| 229 | LI:1091903.1:2000SEP08 | 482 | LI:1091903.1.orf2:2000SEP08 |
| 230 | LI:1169219.1:2000SEP08 | 483 | LI:1169219.1.orf1:2000SEP08 |
| 231 | LI:2050313.1:2000SEP08 | 484 | LI:2050313.1.orf2:2000SEP08 |
| 232 | LI:209351.3:2000SEP08 | 485 | LI:209351.3.orf2:2000SEP08 |
| 232 | LI:209351.3:2000SEP08 | 486 | LI:209351.3.orf3:2000SEP08 |
| 233 | LI:119900.1:2000SEP08 | 487 | LI:119900.1.orf2:2000SEP08 |
| 234 | LI:2052274.1:2000SEP08 | 488 | LI:2052274.1.orf1:2000SEP08 |
| 235 | LI:1075502.1:2000SEP08 | 489 | LI:1075502.1.orf2:2000SEP08 |
| 236 | LI:813697.1:2000SEP08 | 490 | LI:813697.1.orf3:2000SEP08 |
| 237 | LI:814261.1:2000SEP08 | 491 | LI:814261.1.orf3:2000SEP08 |
| 238 | LI:775334.1:2000SEP08 | 492 | LI:775334.1.orf1:2000SEP08 |
| 239 | LI:1180325.1:2000SEP08 | 493 | LI:1180325.1.orf3:2000SEP08 |
| 240 | LI:1183147.3:2000SEP08 | 494 | LI:1183147.3.orf2:2000SEP08 |
| 241 | LI:1175373.3:2000SEP08 | 495 | LI:1175373.3.orf2:2000SEP08 |
| 242 | LI:813757.1:2000SEP08 | 496 | LI:813757.1.orf1:2000SEP08 |
| 243 | LI:1182979.2:2000SEP08 | 497 | LI:1182979.2.orf3:2000SEP08 |
| 244 | LI:1177823.2:2000SEP08 | 498 | LI:1177823.2.orf2:2000SEP08 |
| 245 | LI:1174279.1:2000SEP08 | 499 | LI:1174279.1.orf3:2000SEP08 |
| 246 | LI:1178411.1:2000SEP08 | 500 | LI:1178411.1.orf1:2000SEP08 |
| 247 | LI:1182739.1:2000SEP08 | 501 | LI:1182739.1.orf3:2000SEP08 |
| 248 | LI:234937.4:2000SEP08 | 502 | LI:234937.4.orf1:2000SEP08 |
| 249 | LI:1170660.1:2000SEP08 | 503 | LI:1170660.1.orf2:2000SEP08 |
| 250 | LI:1144409.1:2000SEP08 | 504 | LI:1144409.1.orf1:2000SEP08 |
| 251 | LI:246290.10:2000SEP08 | 505 | LI:246290.10.orf1:2000SEP08 |
| 252 | LI:280034.1:2000SEP08 | 506 | LI:280034.1.orf2:2000SEP08 |

TABLE 2

| SEQ ID NO: | Template ID | GI Number | Probability Score | Annotation |
|---|---|---|---|---|
| 1 | LG:150318.1:2000SEP08 | g11643581 | 0 | Homo sapiens PR- |
| | | | | domain containing |
| | | | | protein 14 |
| | | | | (PRDM14) mRNA, |
| 2 | LG:022529.1:2000SEP08 | g10047272 | 0 | Homo sapiens |
| | | | | mRNA for KIAA1599 |
| | | | | protein, partial cds. |
| 3 | LG:352559.1:2000SEP08 | g13560887 | 1.00E−11 | Homo sapiens EZFIT- |
| | | | | related protein 1 |
| | | | | mRNA, complete |
| 4 | LG:175223.1:2000SEP08 | g10433955 | 1.00E−43 | (fl)(Homo sapiens) |
| | | | | unnamed protein |
| 5 | LG:476989.1:2000SEP08 | g12407394 | 0 | Homo sapiens |
| | | | | tripartite motif |
| | | | | protein TRIM7 |
| | | | | (TRIM7) mRNA, |
| 6 | LG:253268.7:2000SEP08 | g12239368 | 0 | Homo sapiens LYST- |
| | | | | interacting protein |
| | | | | LIP9 mRNA, partial |
| 7 | LG:401322.1:2000SEP08 | g13623682 | 5.00E−26 | Homo sapiens, |
| | | | | tubulin alpha 1, |
| | | | | clone MGC: 2321, |
| 8 | LG:1328436.1:2000SEP08 | g4589587 | 2.00E−55 | Homo sapiens |
| | | | | mRNA for KIAA0972 |
| | | | | protein, complete |
| 9 | LG:475404.1:2000SEP08 | g10434194 | 0 | Homo sapiens |
| | | | | cDNA FLJ12606 fis, |
| | | | | clone |
| | | | | NT2RM4001483, |
| | | | | moderately similar |
| 10 | LG:1384132.1:2000SEP08 | g14042849 | 1.00E−71 | Homo sapiens |
| | | | | cDNA FLJ14959 fis, |
| | | | | clone |
| | | | | PLACE4000156, |
| | | | | moderately similar |
| 11 | LG:410804.18:2000SEP08 | g13543325 | 3.00E−74 | Homo sapiens, |
| | | | | hypothetical |
| | | | | protein MGC8407, |
| | | | | clone MGC: 1820, |
| | | | | mRNA, complete |
| 12 | LG:1082306.1:2000SEP08 | g13325336 | 0 | Homo sapiens, |
| | | | | clone MGC: 10520, |
| | | | | mRNA, complete |
| 13 | LG:233814.4:2000SEP08 | g10434873 | 1.00E−166 | Homo sapiens |
| | | | | cDNA FLJ13044 fis, |
| | | | | clone |
| | | | | NT2RP3001355, |
| | | | | weakly similar to |
| | | | | TRICARBOXYLATE |
| | | | | TRANSPORT |
| 14 | LG:977478.5:2000SEP08 | g12698000 | 0 | Homo sapiens |
| | | | | mRNA for KIAA1728 |
| | | | | protein, partial cds. |
| 15 | LG:025931.1:2000SEP08 | g10436359 | 0 | Homo sapiens |
| | | | | cDNA FLJ14011 fis, |
| | | | | clone |
| | | | | Y79AA1002472, |
| | | | | weakly similar to |
| 16 | LG:885368.1:2000SEP08 | g440824 | 9.00E−60 | (fl)(Arabidopsis |
| | | | | thaliana) ribosomal |
| 17 | LG:1054900.1:2000SEP08 | g12052982 | 0 | Homo sapiens |
| | | | | mRNA; cDNA |
| | | | | DKFZp434l1610 |
| | | | | (from clone |
| | | | | DKFZp434l1610); |
| 18 | LG:995186.2:2000SEP08 | g12052982 | 0 | Homo sapiens |
| | | | | mRNA; cDNA |
| | | | | DKFZp434l1610 |
| | | | | (from clone |
| | | | | DKFZp434l1610); |
| 19 | LG:435048.23:2000SEP08 | g10432866 | 1.00E−177 | Homo sapiens |
| | | | | cDNA FLJ11583 fis, |
| | | | | clone |
| | | | | HEMBA1003680, |
| | | | | weakly similar to |
| | | | | PUTATIVE |
| | | | | AMINOPEPTIDASE |
| | | | | ZK353.6 IN |
| 20 | LG:954859.1:2000SEP08 | g10438224 | 1.00E−176 | Homo sapiens |
| | | | | cDNA: FLJ21990 fis, |
| | | | | clone HEP06386. |
| 21 | LG:364370.1:2000SEP08 | g14043150 | 1.00E−163 | Homo sapiens, |
| | | | | ribosomal protein |
| | | | | L13, clone |
| | | | | MGC: 15490, mRNA, |
| 22 | LG:1098789.1:2000SEP08 | g14029703 | 1.00E−137 | Homo sapiens |
| | | | | myosin regulatory |
| | | | | light chain 2 |
| | | | | (MRLC2) mRNA, |
| 23 | LG:201540.2:2000SEP08 | g12407386 | 0 | Homo sapiens |
| | | | | tripartite motif |
| | | | | protein TRIM5 |
| | | | | isoform delta |
| | | | | (TRIM5) mRNA, |
| 24 | LG:1077357.1:2000SEP08 | g10436361 | 4.00E−66 | Homo sapiens |
| | | | | cDNA FLJ14012 fis, |
| | | | | clone |
| | | | | Y79AA1002482, |
| | | | | moderately similar |
| 25 | LG:1048846.4:2000SEP08 | g10439974 | 1.00E−154 | Homo sapiens |
| | | | | cDNA: FLJ23327 fis, |
| | | | | clone HEP12630, |
| | | | | highly similar to |
| | | | | HSZNF37 Homo |
| | | | | sapiens ZNF37A |
| 26 | LG:336685.1:2000SEP08 | g12697317 | 0 | Homo sapiens |
| | | | | partial mRNA for |
| 27 | LG:1076253.1:2000SEP08 | g7959276 | 5.00E−34 | Homo sapiens |
| | | | | mRNA for KIAA1508 |
| | | | | protein, partial cds. |
| 28 | LG:1400601.2:2000SEP08 | g14042292 | 0 | Homo sapiens |
| | | | | cDNA FLJ14636 fis, |
| | | | | clone |
| | | | | NT2RP2001233, |
| | | | | weakly similar to |
| 29 | LG:1079092.3:2000SEP08 | g12052982 | 6.00E−07 | Homo sapiens |
| | | | | mRNA; cDNA |
| | | | | DKFZp434l1610 |
| | | | | (from clone |
| | | | | DKFZp434l1610); |
| 30 | LG:1086064.1:2000SEP08 | g10436361 | 3.00E−76 | Homo sapiens |
| | | | | cDNA FLJ14012 fis, |
| | | | | clone |
| | | | | Y79AA1002482, |
| | | | | moderately similar |
| 31 | LG:1400608.1:2000SEP08 | g12052731 | 1.00E−175 | Homo sapiens |
| | | | | mRNA; cDNA |
| | | | | DKFZp761G18121 |
| | | | | (from clone |
| | | | | DKFZp761G18121); |
| 32 | LG:399275.5:2000SEP08 | g14042034 | 0 | Homo sapiens |
| | | | | cDNA FLJ14486 fis, |
| | | | | clone |
| | | | | MAMMA1002650, |
| | | | | weakly similar to |
| 33 | LG:293943.1:2000SEP08 | g7022603 | 2.00E−24 | (fl)(Homo sapiens) |
| | | | | unnamed protein |
| 34 | LG:345884.1:2000SEP08 | g13591711 | 0 | Homo sapiens |
| | | | | immunoglobulin |
| | | | | receptor |
| | | | | translocation |
| | | | | associated protein |
| | | | | 2b (IRTA2) mRNA, |
| 35 | LG:400967.1:2000SEP08 | g10047182 | 1.00E−176 | Homo sapiens |
| | | | | mRNA for KIAA1559 |
| | | | | protein, partial cds. |
| 36 | LG:024556.6:2000SEP08 | g11999276 | 0 | Homo sapiens |
| | | | | solute carrier |
| | | | | (SLC25A18) mRNA, |
| | | | | complete cds; |
| | | | | nuclear gene for |
| 37 | LG:081189.3:2000SEP08 | g14325768 | 0 | Homo sapiens |
| | | | | mRNA for KIAA1776 |
| | | | | protein (fibrillin3), |
| 38 | LG:018258.1:2000SEP08 | g12832288 | 3.00E−97 | 0(Mus musculus) |
| 39 | LG:450399.3:2000SEP08 | g13097599 | 1.00E−10 | Homo sapiens, |
| | | | | Similar to ribosomal |
| | | | | protein L23, clone |
| | | | | IMAGE: 3606198, |
| 40 | LG:451122.1:2000SEP08 | g6002102 | 2.00E−38 | (fl)(Digitalis lanata) |
| | | | | Acyl-CoA binding |
| | | | | protein (ACBP) |
| 41 | LG:451682.1:2000SEP08 | g8671496 | 1.00E−134 | (fl)(Oryza sativa) |
| | | | | alpha 3 subunit of |
| | | | | 20S proteasome |
| 42 | LG:238631.4:2000SEP08 | g12803994 | 0 | Homo sapiens, |
| | | | | Similar to HLA class |
| | | | | II region expressed |
| | | | | gene KE2, clone |
| | | | | MGC: 4178, mRNA, |
| 43 | LG:236654.1:2000SEP08 | g11558487 | 2.00E−08 | Homo sapiens |
| | | | | mRNA for B-cell |
| | | | | lymphoma/leukaemia |
| | | | | 11B (BCL11B |
| 44 | LG:332655.1:2000SEP08 | g12856559 | 7.00E−93 | 0(Mus musculus) |
| 45 | LG:217396.2:2000SEP08 | g10047294 | 0 | Homo sapiens |
| | | | | mRNA for KIAA1610 |
| | | | | protein, partial cds. |
| 46 | LG:090574.1:2000SEP08 | g12845416 | 5.00E−37 | 0(Mus musculus) |
| 47 | LG:202943.1:2000SEP08 | g12060829 | 0 | Homo sapiens |
| | | | | serologically |
| | | | | defined breast |
| | | | | cancer antigen NY- |
| | | | | BR-38 mRNA, |
| 48 | LG:236928.1:2000SEP08 | g14388574 | 0 | Macaca |
| | | | | fascicularis brain |
| | | | | cDNA clone: QtrA- |
| 49 | LG:215169.2:2000SEP08 | g790349 | 1.00E−22 | Homo sapiens |
| | | | | (clone NE68) gene |
| 50 | LG:410726.1:2000SEP08 | g9963805 | 2.00E−17 | Homo sapiens zinc |
| | | | | finger protein |
| | | | | ZNF287 (ZNF287) |
| 51 | LG:234372.2:2000SEP08 | g10436854 | 0 | Homo sapiens |
| | | | | cDNA: FLJ20896 fis, |
| | | | | clone ADKA03527. |
| 52 | LG:022629.1:2000SEP08 | g13879442 | 1.00E−178 | (fl)(Mus musculus) |
| | | | | Similar to RIKEN |
| | | | | cDNA 2310035M22 |
| 53 | LG:068682.1:2000SEP08 | g13898616 | 0 | Homo sapiens |
| | | | | serine/threonine |
| | | | | protein kinase SSTK |
| | | | | (SSTK) mRNA. |
| 54 | LG:222335.1:2000SEP08 | g14250137 | 0 | Homo sapiens, |
| | | | | Similar to RIKEN |
| | | | | cDNA 5730421E18 |
| | | | | gene, clone |
| 55 | LG:331342.1:2000SEP08 | g10434081 | 0 | Homo sapiens |
| | | | | cDNA FLJ12538 fis, |
| | | | | clone |
| | | | | NT2RM4000356, |
| | | | | moderately similar |
| | | | | to RAS-RELATED |
| 56 | LG:021770.1:2000SEP08 | g9408105 | 2.00E−47 | Homo sapiens dNT- |
| | | | | 2 gene for |
| | | | | mitochondrial 5′(3′) |
| | | | | deoxyribonucleotid |
| 57 | LG:181607.9:2000SEP08 | g12834244 | 8.00E−91 | 0(Mus musculus) |
| 58 | LG:1042768.1:2000SEP08 | g13937982 | 1.00E−180 | Homo sapiens, |
| | | | | translocase of inner |
| | | | | mitochondrial |
| | | | | membrane 17 |
| | | | | (yeast) homolog A, |
| | | | | clone MGC: 14756, |
| 59 | LG:282729.1:2000SEP08 | g12314268 | 1.00E−107 | (5′ incom)(Homo |
| | | | | sapiens) dJ14N1.2 |
| | | | | (novel S-100/ICaBP |
| | | | | type calcium |
| | | | | binding domain |
| 60 | LG:998305.3:2000SEP08 | g12053098 | 6.00E−88 | Homo sapiens |
| | | | | mRNA; cDNA |
| | | | | DKFZp434A171 |
| | | | | (from clone |
| | | | | DKFZp434A171); |
| 61 | LG:1135213.1:2000SEP08 | g6692607 | 2.00E−69 | (fl)(Mus musculus) |
| | | | | MGA protein |
| 62 | LG:267762.1:2000SEP08 | g12854977 | 1.00E−135 | 0(Mus musculus) |
| 63 | LG:120744.1:2000SEP08 | g12052773 | 0 | Homo sapiens |
| | | | | mRNA; cDNA |
| | | | | DKFZp564B052 |
| | | | | (from clone |
| | | | | DKFZp564B052); |
| 64 | LG:403409.1:2000SEP08 | g8896163 | 0 | Homo sapiens |
| | | | | kinesin-like protein |
| | | | | GAKIN mRNA, |
| 65 | LG:226874.3:2000SEP08 | g3688393 | 3.00E−05 | Homo sapiens |
| | | | | mRNA for triple LIM |
| 66 | LG:1045521.4:2000SEP08 | g10436742 | 4.00E−58 | Homo sapiens |
| | | | | cDNA FLJ14310 fis, |
| | | | | clone |
| 67 | LG:275876.1:2000SEP08 | g5912051 | 6.00E−30 | (3′ and 5′ |
| | | | | incom)(Homo |
| | | | | sapiens) |
| 68 | LG:475127.7:2000SEP08 | g7294107 | 6.00E−44 | (fl)(Drosophila |
| | | | | melanogaster) |
| | | | | CG4638 gene |
| 69 | LG:157263.1:2000SEP08 | g3047402 | 3.00E−40 | (fl)(Homo sapiens) |
| | | | | monocarboxylate |
| | | | | transporter 2 |
| 70 | LG:247382.7:2000SEP08 | g4240292 | 5.00E−25 | Homo sapiens |
| | | | | mRNA for KIAA0902 |
| | | | | protein, complete |
| 71 | LG:197367.5:2000SEP08 | g10172680 | 7.00E−14 | (fl)(Bacillus |
| | | | | halodurans) stage |
| | | | | V sporulation |
| | | | | protein C (peptidyl- |
| 72 | LG:218090.5:2000SEP08 | g9295344 | 0 | Homo sapiens |
| | | | | HSKM-B (HSKM-B) |
| | | | | mRNA, complete |
| 73 | LG:216612.4:2000SEP08 | g8655677 | 0 | Homo sapiens |
| | | | | mRNA; cDNA |
| | | | | DKFZp547M236 |
| | | | | (from clone |
| 74 | LG:197614.1:2000SEP08 | g13358641 | 0 | Macaca |
| | | | | fascicularis brain |
| 75 | LG:378428.1:2000SEP08 | g7264026 | 0 | (fl)(Homo sapiens) |
| | | | | dJ876B10.2 (novel |
| | | | | protein (ortholog of |
| 76 | LG:286639.1:2000SEP08 | g13383264 | 0 | Homo sapiens |
| | | | | mRNA for actin |
| | | | | related protein, |
| 77 | LG:389870.1:2000SEP08 | g14388335 | 9.00E−63 | Macaca |
| | | | | fascicularis brain |
| | | | | cDNA clone: QflA- |
| 78 | LG:1387485.6:2000SEP08 | g10435209 | 0 | Homo sapiens |
| | | | | cDNA FLJ13261 fis, |
| | | | | clone |
| | | | | OVARC1000885, |
| | | | | weakly similar to |
| | | | | OXIDOREDUCTASE |
| 79 | LG:230151.1:2000SEP08 | g8655647 | 0 | Homo sapiens |
| | | | | mRNA; cDNA |
| | | | | DKFZp762M115 |
| | | | | (from clone |
| 80 | LG:215158.5:2000SEP08 | g10440162 | 0 | Homo sapiens |
| | | | | cDNA: FLJ23465 fis, |
| | | | | clone HSI10904. |
| 81 | LG:235840.1:2000SEP08 | g14042343 | 0 | Homo sapiens |
| | | | | cDNA FLJ14666 fis, |
| | | | | clone |
| | | | | NT2RP2003000, |
| | | | | weakly similar to |
| | | | | TUMOR NECROSIS |
| | | | | FACTOR, ALPHA- |
| 82 | LG:350272.1:2000SEP08 | g13477234 | 0 | Homo sapiens, |
| | | | | Similar to RIKEN |
| | | | | cDNA 0610037N03 |
| | | | | gene, clone |
| 83 | LG:232190.1:2000SEP08 | g10434968 | 1.00E−143 | Homo sapiens |
| | | | | cDNA FLJ13105 fis, |
| | | | | clone |
| | | | | NT2RP3002351, |
| | | | | weakly similar to |
| | | | | Human mRNA for |
| | | | | NAD-dependent |
| | | | | methylene |
| | | | | tetrahydrofolate |
| 84 | LG:1068127.1:2000SEP08 | g10436724 | 1.00E−140 | Homo sapiens |
| | | | | cDNA FLJ14297 fis, |
| | | | | clone |
| 85 | LG:408751.3:2000SEP08 | g8886024 | 0 | Homo sapiens |
| | | | | collapsin response |
| | | | | mediator protein-5 |
| | | | | (CRMP5) mRNA, |
| 86 | LG:1078933.1:2000SEP08 | g14042843 | 0 | Homo sapiens |
| | | | | cDNA FLJ14954 fis, |
| | | | | clone |
| | | | | PLACE3000169, |
| | | | | weakly similar to |
| 87 | LG:958731.1:2000SEP08 | g9758769 | 6.00E−88 | (fl) (Arabidopsis |
| | | | | thaliana) 11-beta- |
| | | | | hydroxysteroid |
| 88 | LG:024125.5:2000SEP08 | g12052958 | 0 | Homo sapiens |
| | | | | mRNA; cDNA |
| | | | | DKFZp566J2046 |
| | | | | (from clone |
| | | | | DKFZp566J2046); |
| 89 | LG:373637.3:2000SEP08 | g11118740 | 0 | Homo sapiens |
| | | | | UGT1 gene locus, |
| | | | | complete |
| 90 | LG:1053229.1:2000SEP08 | g12804322 | 0 | Homo sapiens, |
| | | | | clone MGC: 4054, |
| | | | | mRNA, complete |
| 91 | LG:248364.1:2000SEP08 | g12847599 | 0 | 0(Mus musculus) |
| 92 | LG:477130.1:2000SEP08 | g6650751 | 1.00E−55 | (fl)(Ceratopteris |
| | | | | richardii) ribosomal |
| 93 | LG:113786.17:2000SEP08 | g10440515 | 0 | Homo sapiens |
| | | | | mRNA for FLJ00106 |
| | | | | protein, partial cds. |
| 94 | LG:347635.1:2000SEP08 | g11527996 | 0 | Homo sapiens |
| | | | | NOTCH2 protein |
| | | | | (NOTCH2) mRNA, |
| 95 | LG:242966.4:2000SEP08 | g9955987 | 0 | Homo sapiens |
| | | | | clone |
| | | | | TCCCIA00164 |
| 96 | LG:217814.1:2000SEP08 | g10438977 | 0 | Homo sapiens |
| | | | | cDNA: FLJ22551 fis, |
| | | | | clone HSI00804. |
| 97 | LG:476452.1:2000SEP08 | g4126809 | 1.00E−121 | (fl)(Oryza sativa) |
| | | | | glyoxalase I |
| 98 | LG:1100657.1:2000SEP08 | g340298 | 1.00E−107 | Human vasopressin |
| | | | | mRNA, complete |
| 99 | LG:1132418.2:2000SEP08 | g12653472 | 2.00E−86 | Homo sapiens, |
| | | | | proteasome |
| | | | | (prosome, |
| | | | | macropain) |
| | | | | subunit, beta type, |
| 100 | LG:1098570.1:2000SEP08 | g35069 | 1.00E−171 | H. sapiens RNA for |
| | | | | nm23-H2 gene. |
| 101 | LG:1097987.1:2000SEP08 | g9368838 | 0 | Homo sapiens |
| | | | | mRNA; cDNA |
| | | | | DKFZp547l014 (from |
| | | | | clone |
| 102 | LG:337818.2:2000SEP08 | g14042395 | 0 | Homo sapiens |
| | | | | cDNA FLJ14699 fis, |
| | | | | clone |
| | | | | NT2RP2006571, |
| | | | | moderately similar |
| | | | | to CYTOCHROME |
| 103 | LG:1040582.1:2000SEP08 | g13529277 | 1.00E−119 | Homo sapiens, |
| | | | | aldo-keto |
| | | | | reductase family 1, |
| | | | | member A1 |
| | | | | (aldehyde |
| | | | | reductase), clone |
| 104 | LG:1099122.1:2000SEP08 | g13097716 | 8.00E−86 | Homo sapiens, |
| | | | | guanine |
| | | | | nucleotide binding |
| | | | | protein (G protein), |
| | | | | gamma 5, clone |
| 105 | LG:1327449.1:2000SEP08 | g14042109 | 2.00E−96 | Homo sapiens |
| | | | | cDNA FLJ14531 fis, |
| | | | | clone |
| | | | | NT2RM2000371, |
| | | | | weakly similar to |
| | | | | POLYRIBONUCLEOTIDE |
| 106 | LG:227933.5:2000SEP08 | g9280028 | 0 | Macaca |
| | | | | fascicularis brain |
| 107 | LG:1043709.2:2000SEP08 | g12804588 | 4.00E−30 | Homo sapiens, |
| | | | | Similar to CG9172 |
| | | | | gene product, |
| | | | | clone MGC: 886, |
| 108 | LG:1099871.1:2000SEP08 | g12652698 | 1.00E−154 | Homo sapiens, |
| | | | | purine-rich element |
| | | | | binding protein B, |
| | | | | clone MGC: 1947, |
| 109 | LG:1399139.4:2000SEP08 | g10437077 | 0 | Homo sapiens |
| | | | | cDNA: FLJ21069 fis, |
| | | | | clone CAS01594. |
| 110 | LG:236386.1:2000SEP08 | g13477131 | 6.00E−87 | (fl)(Homo sapiens) |
| | | | | SH3 and PX |
| | | | | domain-containing |
| 111 | LG:1015157.1:2000SEP08 | g1881781 | 5.00E−72 | glyoxalase I |
| | | | | (human, HeLa cells, |
| | | | | mRNA Partial, 572 |
| 112 | LG:1065433.1:2000SEP08 | g4589587 | 8.00E−29 | Homo sapiens |
| | | | | mRNA for KIAA0972 |
| | | | | protein, complete |
| 113 | LG:236992.4:2000SEP08 | g12248381 | 0 | Homo sapiens |
| | | | | mRNA for SEMB, |
| 114 | LG:1071124.1:2000SEP08 | g13543418 | 0 | Homo sapiens, |
| | | | | Similar to zinc finger |
| | | | | protein 304, clone |
| | | | | MGC: 4079, mRNA, |
| 115 | LG:206425.2:2000SEP08 | g12052883 | 2.00E−90 | Homo sapiens |
| | | | | mRNA; cDNA |
| | | | | DKFZp564C2478 |
| | | | | (from clone |
| | | | | DKFZp564C2478); |
| 116 | LG:885747.2:2000SEP08 | g12804504 | 2.00E−49 | Homo sapiens, |
| | | | | Similar to ribosomal |
| | | | | protein L31, clone |
| | | | | MGC: 1641, mRNA, |
| 117 | LG:1140501.1:2000SEP08 | g12052919 | 0 | Homo sapiens |
| | | | | mRNA; cDNA |
| | | | | DKFZp564l1782 |
| | | | | (from clone |
| | | | | DKFZp564l1782); |
| 118 | LG:001239.1:2000SEP08 | g14164612 | 0 | Homo sapiens sialic |
| | | | | acid binding |
| | | | | immunoglobulin- |
| | | | | like lectin 10 |
| 119 | LG:018980.1:2000SEP08 | g10435149 | 0 | Homo sapiens |
| | | | | cDNA FLJ13220 fis, |
| | | | | clone |
| | | | | NT2RP4002047, |
| | | | | moderately similar |
| | | | | to GTP-BINDING |
| 120 | LG:1083120.3:2000SEP08 | g515786 | 3.00E−66 | H. sapiens (MAR7) |
| | | | | chromosome 19 |
| | | | | DNA, 302bp. |
| 121 | LG:233258.3:2000SEP08 | g10433125 | 0 | Homo sapiens |
| | | | | cDNA FLJ11790 fis, |
| | | | | clone |
| 122 | LG:999062.1:2000SEP08 | g9759463 | 3.00E−60 | (fl)(Arabidopsis |
| | | | | thaliana) 40S |
| 123 | LG:887776.1:2000SEP08 | g342294 | 1.00E−66 | Macaca mulatta |
| | | | | serum albumin |
| 124 | LG:1400301.2:2000SEP08 | g12751104 | 1.00E−23 | Homo sapiens |
| | | | | PNAS-130 mRNA, |
| 125 | LG:1329362.1:2000SEP08 | g14042849 | 0 | Homo sapiens |
| | | | | cDNA FLJ14959 fis, |
| | | | | clone |
| | | | | PLACE4000156, |
| | | | | moderately similar |
| 126 | LG:1096498.1:2000SEP08 | g13097206 | 1.00E−110 | Homo sapiens, |
| | | | | ribosomal protein, |
| | | | | large, P1, clone |
| | | | | MGC: 5215, mRNA, |
| 127 | LG:1096337.1:2000SEP08 | g13097206 | 1.00E−121 | Homo sapiens, |
| | | | | ribosomal protein, |
| | | | | large, P1, clone |
| | | | | MGC: 5215, mRNA, |
| 128 | LG:1400579.1:2000SEP08 | g10437945 | 4.00E−61 | Homo sapiens |
| | | | | cDNA: FLJ21781 fis, |
| | | | | clone HEP00223. |
| 129 | LG:1080091.1:2000SEP08 | g10436724 | 1.00E−154 | Homo sapiens |
| | | | | cDNA FLJ14297 fis, |
| | | | | clone |
| 130 | LG:1082203.1:2000SEP08 | g10439929 | 0 | Homo sapiens |
| | | | | cDNA: FLJ23296 fis, |
| | | | | clone HEP10656. |
| 131 | LG:1084051.1:2000SEP08 | g6807586 | 1.00E−104 | Novel human gene |
| | | | | mapping to |
| | | | | chomosome 1. |
| 132 | LG:1082393.1:2000SEP08 | g10954043 | 0 | Homo sapiens |
| | | | | KRAB zinc finger |
| | | | | protein ZFQR |
| 133 | LG:1086183.1:2000SEP08 | g12655164 | 6.00E−58 | Homo sapiens, zinc |
| | | | | finger protein 256, |
| | | | | clone MGC: 1413, |
| | | | | mRNA, complete |
| 134 | LG:1090268.1:2000SEP08 | g13938479 | 0 | Homo sapiens, |
| | | | | Similar to |
| | | | | hypothetical |
| | | | | protein FLJ22301, |
| | | | | clone |
| 135 | LG:1400597.5:2000SEP08 | g14250145 | 1.00E−156 | Homo sapiens, |
| | | | | hypothetical |
| | | | | protein FLJ23407, |
| | | | | clone MGC: 14819, |
| | | | | mRNA, complete |
| 136 | LG:1080307.2:2000SEP08 | g14043840 | 2.00E−34 | Homo sapiens, |
| | | | | clone MGC: 14429, |
| | | | | mRNA, complete |
| 137 | LG:1400603.2:2000SEP08 | g4589565 | 4.00E−31 | Homo sapiens |
| | | | | mRNA for KIAA0961 |
| | | | | protein, complete |
| 138 | LG:1052984.1:2000SEP08 | g13937998 | 0 | Homo sapiens, |
| | | | | Similar to DNA- |
| | | | | binding protein, |
| | | | | clone MGC: 14780, |
| 139 | LG:1091259.1:2000SEP08 | g10436675 | 0 | Homo sapiens |
| | | | | cDNA FLJ14260 fis, |
| | | | | clone |
| | | | | PLACE1001118. |
| | | | | weakly similar to |
| 140 | LG:1082263.2:2000SEP08 | g13560887 | 5.00E−13 | Homo sapiens EZFIT- |
| | | | | related protein 1 |
| | | | | mRNA, complete |
| 141 | LG:1048604.2:2000SEP08 | g14249843 | 6.00E−67 | Homo sapiens, |
| | | | | Similar to |
| | | | | hypothetical |
| | | | | protein FLJ23233, |
| | | | | clone MGC: 14876, |
| 142 | LG:1085254.3:2000SEP08 | g10437559 | 0 | Homo sapiens |
| | | | | cDNA: FLJ21457 fis, |
| | | | | clone COL04705. |
| 143 | LG:1400606.2:2000SEP08 | g14042549 | 9.00E−54 | Homo sapiens |
| | | | | cDNA FLJ14779 fis, |
| | | | | clone |
| | | | | NT2RP4000398, |
| | | | | moderately similar |
| 144 | LG:1090358.2:2000SEP08 | g10047182 | 6.00E−30 | Homo sapiens |
| | | | | mRNA for KIAA1559 |
| | | | | protein, partial cds. |
| 145 | LG:1079064.2:2000SEP08 | g10434649 | 0 | Homo sapiens |
| | | | | cDNA FLJ12895 fis, |
| | | | | clone |
| | | | | NT2RP2004187, |
| | | | | weakly similar to |
| 146 | LG:1076866.1:2000SEP08 | g10436460 | 0 | Homo sapiens |
| | | | | cDNA FLJ14087 fis, |
| | | | | clone |
| | | | | MAMMA1000183, |
| | | | | weakly similar to |
| 147 | LG:969359.1:2000SEP08 | g13279004 | 1.00E−120 | Homo sapiens, |
| | | | | ferritin, light |
| | | | | polypeptide, clone |
| | | | | MGC: 10465, mRNA, |
| 148 | LG:366783.1:2000SEP08 | g9651703 | 0 | Homo sapiens |
| | | | | carboxypeptidase |
| | | | | B precursor (CPAH) |
| | | | | mRNA, complete |
| 149 | LG:332176.3:2000SEP08 | g13365900 | 0 | Macaca |
| | | | | fascicularis brain |
| | | | | cDNA clone: QflA- |
| 150 | LG:994938.1:2000SEP08 | g7023331 | 1.00E−76 | Homo sapiens |
| | | | | cDNA FLJ10961 fis, |
| | | | | clone |
| | | | | PLACE1000588, |
| | | | | highly similar to |
| | | | | INTERFERON- |
| 151 | LG:982800.1:2000SEP08 | g12053280 | 0 | Homo sapiens |
| | | | | mRNA; cDNA |
| | | | | DKFZp434J037 |
| | | | | (from clone |
| | | | | DKFZp434J037); |
| 152 | LG:977850.7:2000SEP08 | g10436959 | 5.00E−64 | Homo sapiens |
| | | | | cDNA: FLJ20984 fis, |
| | | | | clone CAE00871. |
| 153 | LG:234748.2:2000SEP08 | g13182754 | 0 | Homo sapiens |
| | | | | HPHRP mRNA, |
| 154 | LG:306284.1:2000SEP08 | g14043648 | 0 | Homo sapiens, |
| | | | | clone MGC: 14161, |
| | | | | mRNA, complete |
| 155 | LI:333170.3:2000SEP08 | g12314083 | 1.00E−93 | (fl)(Homo sapiens) |
| | | | | dJ1007G16.5 (novel |
| | | | | high-mobility group |
| | | | | (nonhistone |
| | | | | chromosomal) |
| 156 | LI:336685.2:2000SEP08 | g12697317 | 0 | Homo sapiens |
| | | | | partial mRNA for |
| 157 | LI:279013.5:2000SEP08 | g12840673 | 3.00E−55 | 0(Mus musculus) |
| 158 | LI:1037075.1:2000SEP08 | g11342540 | 0 | Homo sapiens |
| | | | | mRNA for putative |
| | | | | white family ATP- |
| | | | | binding cassette |
| | | | | transporter (ABCG4 |
| 159 | LI:1073403.1:2000SEP08 | g12804680 | 9.00E−63 | Homo sapiens, |
| | | | | S100 calcium- |
| | | | | binding protein, |
| | | | | beta (neural), |
| | | | | clone MGC: 1323, |
| 160 | LI:1075296.1:2000SEP08 | g12803084 | 1.00E−112 | Homo sapiens, |
| | | | | mitochondrial |
| | | | | ribosomal protein |
| | | | | L12, clone |
| | | | | MGC: 8610, mRNA, |
| 161 | LI:1085501.1:2000SEP08 | g12653784 | 1.00E−140 | Homo sapiens, |
| | | | | clone |
| | | | | IMAGE: 3349601, |
| 162 | LI:1086181.1:2000SEP08 | g3043444 | 1.00E−146 | Homo sapiens |
| | | | | mRNA for EDF-1 |
| 163 | LI:1164493.1:2000SEP08 | g13436439 | 0 | Homo sapiens, |
| | | | | clone MGC: 4400, |
| | | | | mRNA, complete |
| 164 | LI:1175097.1:2000SEP08 | g13937908 | 4.00E−45 | Homo sapiens, |
| | | | | Similar to KIAA0961 |
| | | | | protein, clone |
| | | | | MGC: 12515, mRNA, |
| 165 | LI:1092948.1:2000SEP08 | g12804414 | 2.00E−49 | Homo sapiens, |
| | | | | Similar to |
| | | | | hypothetical |
| | | | | protein FLJ10891, |
| | | | | clone MGC: 925, |
| 166 | LI:380378.2:2000SEP08 | g8655612 | 3.00E−79 | Homo sapiens |
| | | | | mRNA; cDNA |
| | | | | DKFZp762O1415 |
| | | | | (from clone |
| 167 | LI:1029674.1:2000SEP08 | g9651088 | 4.00E−07 | Macaca |
| | | | | fascicularis brain |
| 168 | LI:2048601.3:2000SEP08 | g10434880 | 1.00E−106 | Homo sapiens |
| | | | | cDNA FLJ13048 fis, |
| | | | | clone |
| | | | | NT2RP3001399, |
| | | | | weakly similar to |
| 169 | LI:1186208.1:2000SEP08 | g12052731 | 1.00E−175 | Homo sapiens |
| | | | | mRNA; cDNA |
| | | | | DKFZp761G18121 |
| | | | | (from clone |
| | | | | DKFZp761G18121); |
| 170 | LI:1170753.1:2000SEP08 | g14249995 | 1.00E−101 | Homo sapiens, |
| | | | | clone MGC: 12518, |
| | | | | mRNA, complete |
| 171 | LI:1180908.1:2000SEP08 | g14042849 | 0 | Homo sapiens |
| | | | | cDNA FLJ14959 fis, |
| | | | | clone |
| | | | | PLACE4000156, |
| | | | | moderately similar |
| 172 | LI:1182900.2:2000SEP08 | g10047304 | 0 | Homo sapiens |
| | | | | mRNA for KIAA1615 |
| | | | | protein, partial cds. |
| 173 | LI:1169548.2:2000SEP08 | g14017832 | 0 | Homo sapiens |
| | | | | mRNA for KIAA1808 |
| | | | | protein, partial cds. |
| 174 | LI:1039974.1:2000SEP08 | g13366083 | 0 | Homo sapiens |
| | | | | MARKL1 mRNA for |
| | | | | MAP/microtubule |
| | | | | affinity-regulating |
| | | | | kinase like 1, |
| 175 | LI:1175765.2:2000SEP08 | g13752753 | 5.00E−16 | Homo sapiens zinc |
| | | | | finger 1111 mRNA, |
| | | | | complete cds. |
| 176 | LI:313948.1:2000SEP08 | g9651098 | 0 | Macaca |
| | | | | fascicularis brain |
| 177 | LI:335923.2:2000SEP08 | g11990770 | 3.00E−73 | (fl)(Homo sapiens) |
| | | | | bA534G20.1.1 |
| | | | | (novel protein |
| | | | | similar to Lysozyme |
| | | | | C-1 (1,4-beta-N- |
| | | | | acylmuramidase C, |
| 178 | LI:345884.1:2000SEP08 | g13591713 | 0 | Homo sapiens |
| | | | | immunoglobulin |
| | | | | receptor |
| | | | | translocation |
| | | | | associated protein |
| | | | | 2c (IRTA2) mRNA, |
| 179 | LI:417127.1:2000SEP08 | g12652726 | 1.00E−66 | Homo sapiens, |
| | | | | clone |
| | | | | IMAGE: 3352566, |
| 180 | LI:451710.1:2000SEP08 | g13899057 | 1.00E−61 | (fl)(Mercurialis |
| | | | | annua) ribosomal |
| 181 | LI:406882.2:2000SEP08 | g7019948 | 2.00E−57 | Homo sapiens |
| | | | | cDNA FLJ20081 fis, |
| | | | | clone COL03242. |
| 182 | LI:728223.1:2000SEP08 | g2624328 | 2.00E−44 | (fl)(Oryza sativa) |
| 183 | LI:289783.19:2000SEP08 | g12654714 | 0 | Homo sapiens, |
| | | | | Similar to glucose |
| | | | | regulated protein, |
| | | | | 58 kDa, clone |
| 184 | LI:235255.8:2000SEP08 | g12597311 | 0 | Homo sapiens |
| | | | | clone IMAGE: 72154 |
| | | | | tRNA-guanine |
| | | | | transglycosylase |
| | | | | (TGT) mRNA, |
| 185 | LI:237693.5:2000SEP08 | g12406772 | 4.00E−52 | (fl)(Homo sapiens) |
| | | | | unnamed protein |
| 186 | LI:433670.3:2000SEP08 | g14133250 | 0 | Homo sapiens |
| | | | | mRNA for KIAA1479 |
| | | | | protein, partial cds. |
| 187 | LI:202943.4:2000SEP08 | g12060829 | 0 | Homo sapiens |
| | | | | serologically |
| | | | | defined breast |
| | | | | cancer antigen NY- |
| | | | | BR-38 mRNA, |
| 188 | LI:068682.1:2000SEP08 | g13540325 | 0 | Homo sapiens |
| | | | | serine/threonine |
| | | | | kinase FKSG82 |
| | | | | (FKSG82) mRNA, |
| 189 | LI:203301.3:2000SEP08 | g10047160 | 0 | Homo sapiens |
| | | | | mRNA for KIAA1548 |
| | | | | protein, partial cds. |
| 190 | LI:020726.3:2000SEP08 | g12834087 | 1.00E−157 | 0(Mus musculus) |
| 191 | LI:027209.1:2000SEP08 | g13159480 | 1.00E−127 | (fl)(Homo sapiens) |
| | | | | Translation may |
| | | | | initiate at the ATG |
| | | | | codon at |
| | | | | nucleotides 40-42 |
| 192 | LI:108819.1:2000SEP08 | g12052773 | 0 | Homo sapiens |
| | | | | mRNA; cDNA |
| | | | | DKFZp564B052 |
| | | | | (from clone |
| | | | | DKFZp564B052); |
| 193 | LI:021759.1:2000SEP08 | g12006103 | 0 | Homo sapiens IRA1 |
| | | | | mRNA, complete |
| | | | | cds, alternatively |
| 194 | LI:1165967.1:2000SEP08 | g13529103 | 1.00E−172 | Homo sapiens, |
| | | | | ribosomal protein |
| | | | | S27a, clone |
| | | | | MGC: 12414, mRNA, |
| 195 | LI:1166315.1:2000SEP08 | g12653800 | 1.00E−116 | Homo sapiens, |
| | | | | peptidylprolyl |
| | | | | isomerase A |
| | | | | (cyclophilin A), |
| | | | | clone MGC: 2351, |
| 196 | LI:204626.1:2000SEP08 | g12844770 | 1.00E−138 | 0(Mus musculus) |
| 197 | LI:801140.1:2000SEP08 | g12804322 | 0 | Homo sapiens, |
| | | | | clone MGC: 4054, |
| | | | | mRNA, complete |
| 198 | LI:286639.1:2000SEP08 | g13938318 | 0 | Homo sapiens, |
| | | | | clone MGC: 15664, |
| | | | | mRNA, complete |
| 199 | LI:288905.4:2000SEP08 | g12698056 | 0 | Homo sapiens |
| | | | | mRNA for KIAA1756 |
| | | | | protein, partial cds. |
| 200 | LI:332161.1:2000SEP08 | g14388335 | 2.00E−62 | Macaca |
| | | | | fascicularis brain |
| | | | | cDNA clone: QflA- |
| 201 | LI:184867.1:2000SEP08 | g7209586 | 4.00E−47 | (fl)(Rattus |
| | | | | norvegicus) DA41 |
| 202 | LI:229932.4:2000SEP08 | g10438187 | 0 | Homo sapiens |
| | | | | cDNA: FLJ21963 fis, |
| | | | | clone HEP05583. |
| 203 | LI:1189932.1:2000SEP08 | g12483887 | 0 | Homo sapiens |
| | | | | solute carrier 19A3 |
| | | | | mRNA, complete |
| 204 | LI:1076689.1:2000SEP08 | g12804758 | 6.00E−20 | Homo sapiens, |
| | | | | ribonuclease 6 |
| | | | | precursor, clone |
| | | | | MGC: 3554, mRNA, |
| 205 | LI:415181.2:2000SEP08 | g11762100 | 0 | (fl)(Zea mays) myoinositol |
| | | | | 1- |
| 206 | LI:296358.1:2000SEP08 | g12053148 | 0 | Homo sapiens |
| | | | | mRNA; cDNA |
| | | | | DKFZp434G2226 |
| | | | | (from clone |
| | | | | DKFZp434G2226); |
| 207 | LI:205186.3:2000SEP08 | g567232 | 4.00E−17 | (fl)(Mus musculus) |
| | | | | proline-rich protein |
| 208 | LI:220537.2:2000SEP08 | g13365896 | 0 | Macaca |
| | | | | fascicularis brain |
| | | | | cDNA clone: QflA- |
| 209 | LI:248364.2:2000SEP08 | g12847599 | 0 | 0(Mus musculus) |
| 210 | LI:2048338.1:2000SEP08 | g12848905 | 3.00E−78 | 0(Mus musculus) |
| 211 | LI:1185203.8:2000SEP08 | g11231084 | 3.00E−85 | Macaca |
| | | | | fascicularis brain |
| 212 | LI:021770.3:2000SEP08 | g5931541 | 2.00E−45 | Homo sapiens |
| | | | | genomic DNA, |
| | | | | chromosome |
| | | | | 22q11.2, BCRL2 |
| 213 | LI:1185841.1:2000SEP08 | g14269501 | 0 | Homo sapiens |
| | | | | unconventional |
| | | | | myosin 1G valine |
| | | | | form (MYO1G) |
| | | | | mRNA, MYO1G-V |
| 214 | LI:1181710.1:2000SEP08 | g7959206 | 2.00E−49 | Homo sapiens |
| | | | | mRNA for KIAA1473 |
| | | | | protein, partial cds. |
| 215 | LI:2048959.1:2000SEP08 | g5817101 | 1.00E−16 | Homo sapiens |
| | | | | mRNA; cDNA |
| | | | | DKFZp434G1621 |
| | | | | (from clone |
| 216 | LI:798494.1:2000SEP08 | g10047182 | 2.00E−23 | Homo sapiens |
| | | | | mRNA for KIAA1559 |
| | | | | protein, partial cds. |
| 217 | LI:2049223.1:2000SEP08 | g7959206 | 2.00E−43 | Homo sapiens |
| | | | | mRNA for KIAA1473 |
| | | | | protein, partial cds. |
| 218 | LI:1177833.1:2000SEP08 | g12052982 | 0 | Homo sapiens |
| | | | | mRNA; cDNA |
| | | | | DKFZp434l1610 |
| | | | | (from clone |
| | | | | DKFZp434l1610); |
| 219 | LI:2049267.1:2000SEP08 | g515786 | 6.00E−61 | H. sapiens (MAR7) |
| | | | | chromosome 19 |
| | | | | DNA, 302bp. |
| 220 | LI:1165939.1:2000SEP08 | g7020753 | 9.00E−13 | Homo sapiens |
| | | | | cDNA FLJ20562 fis, |
| | | | | clone KAT11992. |
| 221 | LI:1170958.1:2000SEP08 | g5262556 | 1.00E−14 | Homo sapiens |
| | | | | mRNA; cDNA |
| | | | | DKFZp569D2231 |
| | | | | (from clone |
| | | | | DKFZp569D2231); |
| 222 | LI:1089827.1:2000SEP08 | g14042292 | 0 | Homo sapiens |
| | | | | cDNA FLJ14636 fis, |
| | | | | clone |
| | | | | NT2RP2001233, |
| | | | | weakly similar to |
| 223 | LI:792112.1:2000SEP08 | g10437945 | 4.00E−61 | Homo sapiens |
| | | | | cDNA: FLJ21781 fis, |
| | | | | clone HEP00223. |
| 224 | LI:282219.2:2000SEP08 | g12656630 | 7.00E−63 | Homo sapiens |
| | | | | Kruppel-like zinc |
| | | | | finger protein GLIS2 |
| | | | | mRNA, complete |
| 225 | LI:1088010.2:2000SEP08 | g13623632 | 0 | Homo sapiens, |
| | | | | clone MGC: 13105, |
| | | | | mRNA, complete |
| 226 | LI:1165276.1:2000SEP08 | g6807586 | 1.00E−106 | Novel human gene |
| | | | | mapping to |
| | | | | chomosome 1. |
| 227 | LI:1169524.2:2000SEP08 | g10047182 | 5.00E−30 | Homo sapiens |
| | | | | mRNA for KIAA1559 |
| | | | | protein, partial cds. |
| 228 | LI:1180255.1:2000SEP08 | g14017870 | 0 | Homo sapiens |
| | | | | mRNA for KIAA1827 |
| | | | | protein, partial cds. |
| 229 | LI:1091903.1:2000SEP08 | g14042372 | 2.00E−49 | Homo sapiens |
| | | | | cDNA FLJ14686 fis, |
| | | | | clone |
| | | | | NT2RP2004961, |
| | | | | moderately similar |
| | | | | to Rattus |
| | | | | norvegicus |
| | | | | KRAB/zinc finger |
| 230 | LI:1169219.1:2000SEP08 | g13937998 | 0 | Homo sapiens, |
| | | | | Similar to DNA- |
| | | | | binding protein, |
| | | | | clone MGC: 14780, |
| 231 | LI:2050313.1:2000SEP08 | g12862319 | 0 | Homo sapiens |
| | | | | mRNA for WDC146, |
| 232 | LI:209351.3:2000SEP08 | g14042794 | 0 | Homo sapiens |
| | | | | cDNA FLJ14923 fis, |
| | | | | clone |
| | | | | PLACE1008244, |
| | | | | weakly similar to |
| | | | | VEGETATIBLE |
| 233 | LI:119900.1:2000SEP08 | g14042821 | 2.00E−66 | Homo sapiens |
| | | | | cDNA FLJ14939 fis, |
| | | | | clone |
| | | | | PLACE1010702, |
| | | | | moderately similar |
| 234 | LI:2052274.1:2000SEP08 | g8698839 | 4.00E−07 | Homo sapiens |
| | | | | genomic DNA, |
| | | | | chromosome 8q23, |
| 235 | LI:1075502.1:2000SEP08 | g13960141 | 1.00E−114 | Homo sapiens, |
| | | | | uridine |
| | | | | monophosphate |
| | | | | synthetase (orotate |
| | | | | phosphoribosyl |
| | | | | transferase and |
| | | | | orotidine-5′- |
| | | | | decarboxylase), |
| 236 | LI:813697.1:2000SEP08 | g7020744 | 6.00E−59 | Homo sapiens |
| | | | | cDNA FLJ20557 fis, |
| | | | | clone KAT11869. |
| 237 | LI:814261.1:2000SEP08 | g7023331 | 1.00E−76 | Homo sapiens |
| | | | | cDNA FLJ10961 fis, |
| | | | | clone |
| | | | | PLACE1000588, |
| | | | | highly similar to |
| | | | | INTERFERON- |
| 238 | LI:775334.1:2000SEP08 | g4589587 | 3.00E−23 | Homo sapiens |
| | | | | mRNA for KIAA0972 |
| | | | | protein, complete |
| 239 | LI:1180325.1:2000SEP08 | g10438159 | 0 | Homo sapiens |
| | | | | cDNA: FLJ21941 fis, |
| | | | | clone HEP04524. |
| 240 | LI:1183147.3:2000SEP08 | g10440084 | 0 | Homo sapiens |
| | | | | cDNA: FLJ23407 fis, |
| | | | | clone HEP19601. |
| 241 | LI:1175373.3:2000SEP08 | g7243242 | 5.00E−51 | Homo sapiens |
| | | | | mRNA for KIAA1431 |
| | | | | protein, partial cds. |
| 242 | LI:813757.1:2000SEP08 | g4589587 | 1.00E−31 | Homo sapiens |
| | | | | mRNA for KIAA0972 |
| | | | | protein, complete |
| 243 | LI:1182979.2:2000SEP08 | g14042292 | 2.00E−87 | Homo sapiens |
| | | | | cDNA FLJ14636 fis, |
| | | | | clone |
| | | | | NT2RP2001233, |
| | | | | weakly similar to |
| 244 | LI:1177823.2:2000SEP08 | g12052982 | 0 | Homo sapiens |
| | | | | mRNA; cDNA |
| | | | | DKFZp434l1610 |
| | | | | (from clone |
| | | | | DKFZp434l1610); |
| 245 | LI:1174279.1:2000SEP08 | g10047182 | 1.00E−27 | Homo sapiens |
| | | | | mRNA for KIAA1559 |
| | | | | protein, partial cds. |
| 246 | LI:1178411.1:2000SEP08 | g14042843 | 0 | Homo sapiens |
| | | | | cDNA FLJ14954 fis, |
| | | | | clone |
| | | | | PLACE3000169, |
| | | | | weakly similar to |
| 247 | LI:1182739.1:2000SEP08 | g12655164 | 8.00E−31 | Homo sapiens, zinc |
| | | | | finger protein 256, |
| | | | | clone MGC: 1413, |
| | | | | mRNA, complete |
| 248 | LI:234937.4:2000SEP08 | g12804418 | 0 | Homo sapiens, |
| | | | | clone MGC: 1136, |
| | | | | mRNA, complete |
| 249 | LI:1170660.1:2000SEP08 | g14388419 | 0 | Macaca |
| | | | | fascicularis brain |
| | | | | cDNA clone: QmoA- |
| 250 | LI:1144409.1:2000SEP08 | g12053280 | 0 | Homo sapiens |
| | | | | mRNA; cDNA |
| | | | | DKFZp434J037 |
| | | | | (from clone |
| | | | | DKFZp434J037); |
| 251 | LI:246290.10:2000SEP08 | g10438695 | 0 | Homo sapiens |
| | | | | cDNA: FLJ22347 fis, |
| | | | | clone HRC06188. |
| 252 | LI:280034.1:2000SEP08 | g12847023 | 1.00E−132 | 0(Mus musculus) |

TABLE 3

| SEQ ID NO: | Template ID | Start | Stop | Frame | Pfam Hit | Pfam Description | E-value |
|---|---|---|---|---|---|---|---|
| 1 | LG:150318.1:2000SEP08 | 11 | 79 | forward 2 | zf-C2H2 | Zinc finger, C2H2 type | 2.00E−06 |
| 2 | LG:022529.1:2000SEP08 | 426 | 701 | forward 3 | C2 | C2 domain | 3.30E−06 |
| 3 | LG:352559.1:2000SEP08 | 125 | 313 | forward 2 | KRAB | KRAB box | 1.60E−41 |
| 4 | LG:175223.1:2000SEP08 | 210 | 431 | forward 3 | CSD | ‘Cold-shock’ DNA-binding domain | 1.40E−18 |
| 5 | LG:476989.1:2000SEP08 | 149 | 223 | forward 2 | zf-C3HC4 | Zinc finger, C3HC4 type (RING finger) | 3.00E−10 |
| 6 | LG:253268.7:2000SEP08 | 211 | 435 | forward 1 | rrm | RNA recognition motif. (a.k.a. RRM, | 3.50E−16 |
| | | | | | | RBD, or RNP domain) | |
| 7 | LG:401322.1:2000SEP08 | 156 | 341 | forward 3 | tubulin | Tubulin/FtsZ family | 7.10E−20 |
| 7 | LG:401322.1:2000SEP08 | 371 | 478 | forward 2 | tubulin | Tubulin/FtsZ family | 2.50E−06 |
| 8 | LG:1328436.1:2000SEP08 | 134 | 322 | forward 2 | KRAB | KRAB box | 1.70E−42 |
| 9 | LG:475404.1:2000SEP08 | 176 | 328 | forward 2 | KRAB | KRAB box | 1.10E−15 |
| 10 | LG:1384132.1:2000SEP08 | 157 | 225 | forward 1 | zf-C2H2 | Zinc finger, C2H2 type | 9.00E−04 |
| 11 | LG:410804.18:2000SEP08 | 133 | 243 | forward 1 | pkinase | Protein kinase domain | 7.90E−04 |
| 12 | LG:1082306.1:2000SEP08 | 199 | 378 | forward 1 | KRAB | KRAB box | 4.90E−20 |
| 12 | LG:1082306.1:2000SEP08 | 559 | 627 | forward 1 | zf-C2H2 | Zinc finger, C2H2 type | 9.60E−05 |
| 13 | LG:233814.4:2000SEP08 | 624 | 947 | forward 3 | mito_carr | Mitochondrial carrier protein | 2.50E−04 |
| 14 | LG:977478.5:2000SEP08 | 351 | 449 | forward 3 | ank | Ank repeat | 1.10E−08 |
| 15 | LG:025931.1:2000SEP08 | 660 | 728 | forward 3 | zf-C2H2 | Zinc finger, C2H2 type | 4.50E−06 |
| 15 | LG:025931.1:2000SEP08 | 29 | 97 | forward 2 | zf-C2H2 | Zinc finger, C2H2 type | 7.20E−06 |
| 16 | LG:885368.1:2000SEP08 | 133 | 462 | forward 1 | Ribosomal_S8 | Ribosomal protein S8 | 7.50E−48 |
| 17 | LG:1054900.1:2000SEP08 | 78 | 218 | forward 3 | KRAB | KRAB box | 2.30E−17 |
| 18 | LG:995186.2:2000SEP08 | 151 | 357 | forward 1 | KRAB | KRAB box | 1.10E−04 |
| 19 | LG:435048.23:2000SEP08 | 2 | 457 | forward 2 | Peptidase_M17 | Cytosol aminopeptidase family, | 5.30E−09 |
| | | | | | | catalytic domain | |
| 20 | LG:954859.1:2000SEP08 | 245 | 565 | forward 2 | Ribosomal_L7Ae | Ribosomal protein | 7.80E−17 |
| | | | | | | L7Ae/L30e/S12e/Gadd45 family | |
| 21 | LG:364370.1:2000SEP08 | 42 | 578 | forward 3 | Ribosomal_L13e | Ribosomal protein L13e | 2.30E−137 |
| 22 | LG:1098789.1:2000SEP08 | 45 | 131 | forward 3 | efhand | EF hand | 8.70E−05 |
| 23 | LG:201540.2:2000SEP08 | 479 | 604 | forward 2 | zf-B_box | B-box zinc finger. | 9.60E−14 |
| 23 | LG:201540.2:2000SEP08 | 254 | 385 | forward 2 | zf-C3HC4 | Zinc finger, C3HC4 type (RING finger) | 4.80E−13 |
| 24 | LG:1077357.1:2000SEP08 | 94 | 282 | forward 1 | KRAB | KRAB box | 4.80E−31 |
| 25 | LG:1048846.4:2000SEP08 | 60 | 245 | forward 3 | KRAB | KRAB box | 7.10E−39 |
| 26 | LG:336685.1:2000SEP08 | 945 | 1121 | forward 3 | homeobox | Homeobox domain | 3.10E−11 |
| 27 | LG:1076253.1:2000SEP08 | 6 | 188 | forward 3 | KRAB | KRAB box | 4.10E−14 |
| 27 | LG:1076253.1:2000SEP08 | 939 | 1007 | forward 3 | zf-C2H2 | Zinc finger, C2H2 type | 9.30E−06 |
| 28 | LG:1400601.2:2000SEP08 | 38 | 106 | forward 2 | zf-C2H2 | Zinc finger, C2H2 type | 1.50E−05 |
| 29 | LG:1079092.3:2000SEP08 | 314 | 382 | forward 2 | zf-C2H2 | Zinc finger, C2H2 type | 8.80E−06 |
| 30 | LG:1086064.1:2000SEP08 | 128 | 316 | forward 2 | KRAB | KRAB box | 2.00E−34 |
| 31 | LG:1400608.1:2000SEP08 | 138 | 326 | forward 3 | KRAB | KRAB box | 1.30E−40 |
| 32 | LG:399275.5:2000SEP08 | 328 | 645 | forward 1 | BTB | BTB/POZ domain | 4.10E−18 |
| 33 | LG:293943.1:2000SEP08 | 265 | 327 | forward 1 | IQ | IQ calmodulin-binding motif | 5.10E−04 |
| 34 | LG:345884.1:2000SEP08 | 203 | 355 | forward 2 | ig | Immunoglobulin domain | 7.40E−04 |
| 35 | LG:400967.1:2000SEP08 | 109 | 300 | forward 1 | KRAB | KRAB box | 1.50E−35 |
| 36 | LG:024556.6:2000SEP08 | 526 | 774 | forward 1 | mito_carr | Mitochondrial carrier protein | 2.90E−19 |
| 37 | LG:081189.3:2000SEP08 | 362 | 475 | forward 2 | EGF | EGF-like domain | 3.80E−06 |
| 38 | LG:018258.1:2000SEP08 | 174 | 605 | forward 3 | Nitroreductase | Nitroreductase family | 9.00E−05 |
| 39 | LG:450399.3:2000SEP08 | 81 | 446 | forward 3 | Ribosomal_L14 | Ribosomal protein L14p/L23e | 1.20E−53 |
| 40 | LG:451122.1:2000SEP08 | 49 | 303 | forward 1 | ACBP | Acyl CoA binding protein | 1.70E−51 |
| 41 | LG:451682.1:2000SEP08 | 117 | 560 | forward 3 | proteasome | Proteasome A-type and B-type | 4.40E−59 |
| 42 | LG:238631.4:2000SEP08 | 100 | 453 | forward 1 | KE2 | KE2 family protein | 3.80E−38 |
| 43 | LG:236654.1:2000SEP08 | 810 | 878 | forward 3 | zf-C2H2 | Zinc finger, C2H2 type | 1.20E−04 |
| 43 | LG:236654.1:2000SEP08 | 434 | 502 | forward 2 | zf-C2H2 | Zinc finger, C2H2 type | 6.60E−04 |
| 44 | LG:332655.1:2000SEP08 | 415 | 513 | forward 1 | ank | Ank repeat | 3.10E−10 |
| 45 | LG:217396.2:2000SEP08 | 679 | 882 | forward 1 | ELM2 | ELM2 domain | 5.00E−14 |
| 45 | LG:217396.2:2000SEP08 | 994 | 1134 | forward 1 | myb_DNA-binding | Myb-like DNA-binding domain | 2.00E−11 |
| 46 | LG:090574.1:2000SEP08 | 33 | 245 | forward 3 | carb_anhydrase | Eukaryotic-type carbonic anhydrase | 3.10E−31 |
| 47 | LG:202943.1:2000SEP08 | 199 | 360 | forward 1 | sushi | Sushi domain (SCR repeat) | 3.80E−18 |
| 48 | LG:236928.1:2000SEP08 | 259 | 1101 | forward 1 | GNS1_SUR4 | GNS1/SUR4 family | 9.80E−09 |
| 49 | LG:215169.2:2000SEP08 | 89 | 208 | forward 2 | Idl_recept_a | Low-density lipoprotein receptor | 1.00E−12 |
| | | | | | | domain class A | |
| 50 | LG:410726.1:2000SEP08 | 724 | 912 | forward 1 | KRAB | KRAB box | 2.10E−17 |
| 50 | LG:410726.1:2000SEP08 | 352 | 636 | forward 1 | SCAN | SCAN domain | 8.90E−55 |
| 51 | LG:234372.2:2000SEP08 | 557 | 862 | forward 2 | PH | PH domain | 3.00E−09 |
| 51 | LG:234372.2:2000SEP08 | 950 | 1408 | forward 2 | RhoGAP | RhoGAP domain | 3.30E−39 |
| 51 | LG:234372.2:2000SEP08 | 1952 | 2119 | forward 2 | SH3 | SH3 domain | 4.60E−11 |
| 52 | LG:022629.1:2000SEP08 | 261 | 410 | forward 3 | zf-C3HC4 | Zinc finger, C3HC4 type (RING finger) | 2.00E−07 |
| 53 | LG:068682.1:2000SEP08 | 176 | 883 | forward 2 | pkinase | Protein kinase domain | 1.70E−65 |
| 54 | LG:222335.1:2000SEP08 | 7 | 732 | forward 1 | DUF71 | Domain of unknown function DUF71 | 6.70E−83 |
| 55 | LG:331342.1:2000SEP08 | 558 | 1076 | forward 3 | arf | ADP-ribosylation factor family | 1.20E−04 |
| 55 | LG:331342.1:2000SEP08 | 588 | 1154 | forward 3 | ras | Ras family | 2.60E−79 |
| 56 | LG:021770.1:2000SEP08 | 228 | 1199 | forward 3 | adh_zinc | Zinc-binding dehydrogenases | 7.80E−20 |
| 57 | LG:181607.9:2000SEP08 | 161 | 604 | forward 2 | Josephin | Josephin | 5.00E−50 |
| 58 | LG:1042768.1:2000SEP08 | 42 | 443 | forward 3 | Tim17 | Mitochondrial import inner membrane | 1.40E−82 |
| | | | | | | translocase subunit Tim17 | |
| 59 | LG:282729.1:2000SEP08 | 61 | 192 | forward 1 | S_100 | S-100/ICaBP type calcium binding | 1.10E−12 |
| | | | | | | domain | |
| 60 | LG:998305.3:2000SEP08 | 266 | 364 | forward 2 | ank | Ank repeat | 2.10E−07 |
| 61 | LG:1135213.1:2000SEP08 | 340 | 531 | forward 1 | T-box | T-box | 8.80E−27 |
| 62 | LG:267762.1:2000SEP08 | 64 | 1125 | forward 1 | A_deaminase | Adenosine/AMP deaminase | 7.80E−20 |
| 63 | LG:120744.1:2000SEP08 | 301 | 813 | forward 1 | vwa | von Willebrand factor type A domain | 1.90E−52 |
| 64 | LG:403409.1:2000SEP08 | 1458 | 1652 | forward 3 | FHA | FHA domain | 3.00E−04 |
| 64 | LG:403409.1:2000SEP08 | 78 | 1193 | forward 3 | kinesin | Kinesin motor domain | 6.80E−172 |
| 65 | LG:226874.3:2000SEP08 | 118 | 291 | forward 1 | LIM | LIM domain | 8.40E−13 |
| 66 | LG:1045521.4:2000SEP08 | 160 | 1464 | forward 1 | aminotran_1 | Aminotransferase class-I | 2.00E−10 |
| 67 | LG:275876.1:2000SEP08 | 508 | 831 | forward 1 | CH | Calponin homology (CH) domain | 2.40E−26 |
| 68 | LG:475127.7:2000SEP08 | 169 | 498 | forward 1 | GTP_EFTU | Elongation factor Tu family | 2.20E−46 |
| 69 | LG:157263.1:2000SEP08 | 163 | 1314 | forward 1 | MCT | Monocarboxylate transporter | 6.10E−31 |
| 70 | LG:247382.7:2000SEP08 | 1105 | 1353 | forward 1 | PDZ | PDZ domain (Also known as DHR or | 4.20E−07 |
| | | | | | | GLGF). | |
| 70 | LG:247382.7:2000SEP08 | 487 | 684 | forward 1 | SAM | SAM domain (Sterile alpha motif) | 1.20E−11 |
| 71 | LG:197367.5:2000SEP08 | 50 | 472 | forward 2 | Pept_tRNA_hydro | Peptidyl-tRNA hydrolase | 8.90E−05 |
| 72 | LG:218090.5:2000SEP08 | 179 | 295 | forward 2 | zf-MYND | MYND finger | 3.00E−10 |
| 73 | LG:216612.4:2000SEP08 | 138 | 1274 | forward 3 | Cation_efflux | Cation efflux family | 8.90E−63 |
| 74 | LG:197614.1:2000SEP08 | 1699 | 3048 | forward 1 | Oxysterol_BP | Oxysterol-binding protein | 4.10E−31 |
| 74 | LG:197614.1:2000SEP08 | 886 | 1164 | forward 1 | PH | PH domain | 1.20E−14 |
| 75 | LG:378428.1:2000SEP08 | 621 | 920 | forward 3 | PH | PH domain | 2.00E−06 |
| 76 | LG:286639.1:2000SEP08 | 1294 | 1713 | forward 1 | actin | Actin | 9.10E−64 |
| 76 | LG:286639.1:2000SEP08 | 630 | 1319 | forward 3 | actin | Actin | 1.70E−62 |
| 77 | LG:389870.1:2000SEP08 | 131 | 640 | forward 2 | ras | Ras family | 3.40E−37 |
| 78 | LG:1387485.6:2000SEP08 | 227 | 709 | forward 2 | adh_short | short chain dehydrogenase | 1.10E−25 |
| 79 | LG:230151.1:2000SEP08 | 631 | 1602 | forward 1 | E1_dehydrog | Dehydrogenase E1 component | 1.10E−09 |
| 80 | LG:215158.5:2000SEP08 | 1243 | 1377 | forward 1 | zf-AN1 | AN1-like Zinc finger | 9.10E−04 |
| 81 | LG:235840.1:2000SEP08 | 1241 | 1537 | forward 2 | K_tetra | K+ channel tetramerisation domain | 5.60E−22 |
| 82 | LG:350272.1:2000SEP08 | 1424 | 1780 | forward 2 | SPRY | SPRY domain | 2.10E−10 |
| 82 | LG:350272.1:2000SEP08 | 557 | 682 | forward 2 | zf-C3HC4 | Zinc finger, C3HC4 type (RING finger) | 5.30E−11 |
| 83 | LG:232190.1:2000SEP08 | 497 | 691 | forward 2 | zf-DHHC | DHHC zinc finger domain | 2.30E−38 |
| 84 | LG:1068127.1:2000SEP08 | 158 | 307 | forward 2 | KRAB | KRAB box | 3.00E−12 |
| 85 | LG:408751.3:2000SEP08 | 194 | 1204 | forward 2 | Dihydroorotase | Dihydroorotase-like | 1.20E−12 |
| 85 | LG:408751.3:2000SEP08 | 456 | 1355 | forward 3 | Dihydroorotase | Dihydroorotase-like | 6.60E−08 |
| 86 | LG:1078933.1:2000SEP08 | 373 | 441 | forward 1 | zf-C2H2 | Zinc finger, C2H2 type | 1.20E−07 |
| 86 | LG:1078933.1:2000SEP08 | 689 | 775 | forward 2 | zf-C2H2 | Zinc finger, C2H2 type | 1.00E−03 |
| 87 | LG:958731.1:2000SEP08 | 153 | 707 | forward 3 | adh_short | short chain dehydrogenase | 2.40E−39 |
| 88 | LG:024125.5:2000SEP08 | 132 | 635 | forward 3 | FAA_hydrolase | Fumarylacetoacetate (FAA) hydrolase | 1.20E−83 |
| | | | | | | family | |
| 89 | LG:373637.3:2000SEP08 | 61 | 255 | forward 1 | DnaJ | DnaJ domain | 7.40E−33 |
| 90 | LG:1053229.1:2000SEP08 | 514 | 582 | forward 1 | zf-C2H2 | Zinc finger, C2H2 type | 2.50E−08 |
| 91 | LG:248364.1:2000SEP08 | 253 | 594 | forward 1 | BTB | BTB/POZ domain | 3.60E−04 |
| 91 | LG:248364.1:2000SEP08 | 1544 | 1612 | forward 2 | zf-C2H2 | Zinc finger, C2H2 type | 7.10E−06 |
| 92 | LG:477130.1:2000SEP08 | 174 | 554 | forward 3 | Ribosomal_L13 | Ribosomal protein L13 | 3.60E−37 |
| 93 | LG:113786.17:2000SEP08 | 134 | 376 | forward 2 | PDZ | PDZ domain (Also known as DHR or | 8.10E−15 |
| | | | | | | GLGF). | |
| 94 | LG:347635.1:2000SEP08 | 315 | 410 | forward 3 | EGF | EGF-like domain | 6.70E−09 |
| 94 | LG:347635.1:2000SEP08 | 605 | 709 | forward 2 | EGF | EGF-like domain | 1.10E−04 |
| 94 | LG:347635.1:2000SEP08 | 1057 | 1152 | forward 1 | EGF | EGF-like domain | 1.20E−04 |
| 95 | LG:242966.4:2000SEP08 | 7 | 918 | forward 1 | aldedh | Aldehyde dehydrogenase family | 1.10E−05 |
| 95 | LG:242966.4:2000SEP08 | 1031 | 1993 | forward 2 | aldedh | Aldehyde dehydrogenase family | 2.20E−05 |
| 96 | LG:217814.1:2000SEP08 | 1167 | 1265 | forward 3 | ank | Ank repeat | 6.60E−08 |
| 97 | LG:476452.1:2000SEP08 | 210 | 581 | forward 3 | Glyoxalase | Glyoxalase/Bleomycln resistance | 8.10E−39 |
| | | | | | | protein/Dioxygenase superfamily | |
| 98 | LG:1100657.1:2000SEP08 | 151 | 384 | forward 1 | hormone5 | Neurohypophysial hormones, C- | 8.60E−47 |
| | | | | | | terminal Domain | |
| 99 | LG:1132418.2:2000SEP08 | 109 | 393 | forward 1 | proteasome | Proteasome A-type and B-type | 1.10E−14 |
| 100 | LG:1098570.1:2000SEP08 | 129 | 572 | forward 3 | NDK | Nucleoside diphosphate kinases | 3.70E−117 |
| 101 | LG:1097987.1:2000SEP08 | 747 | 908 | forward 3 | Ribosomal_L23 | Ribosomal protein L23 | 5.80E−14 |
| 102 | LG:337818.2:2000SEP08 | 136 | 1518 | forward 1 | p450 | Cytochrome P450 | 6.30E−175 |
| 103 | LG:1040582.1:2000SEP08 | 266 | 556 | forward 2 | aldo_ket_red | Aldo/keto reductase family | 1.30E−47 |
| 103 | LG:1040582.1:2000SEP08 | 546 | 635 | forward 3 | aldo_ket_red | Aldo/keto reductase family | 7.60E−06 |
| 104 | LG:1099122.1:2000SEP08 | 84 | 248 | forward 3 | G-gamma | GGL domain | 4.90E−31 |
| 105 | LG:1327449.1:2000SEP08 | 461 | 526 | forward 2 | Ribosomal_S8 | Ribosomal protein S8 | 4.80E−07 |
| 105 | LG:1327449.1:2000SEP08 | 246 | 359 | forward 3 | Ribosomal_S8 | Ribosomal protein S8 | 3.00E−06 |
| 106 | LG:227933.5:2000SEP08 | 523 | 1455 | forward 1 | AMP-binding | AMP-binding enzyme | 7.20E−05 |
| 107 | LG:1043709.2:2000SEP08 | 457 | 723 | forward 1 | oxidored_q6 | NADH ubiquinone oxidoreductase, 20 Kd | 8.40E−08 |
| | | | | | | subunit | |
| 108 | LG:1099871.1:2000SEP08 | 34 | 411 | forward 1 | histone | Core histone H2A/H2B/H3/H4 | 4.50E−47 |
| 109 | LG:1399139.4:2000SEP08 | 117 | 233 | forward 3 | CAP_GLY | CAP-Gly domain | 4.30E−10 |
| 110 | LG:236386.1:2000SEP08 | 1383 | 1712 | forward 3 | PX | PX domain | 1.90E−13 |
| 111 | LG:1015157.1:2000SEP08 | 115 | 552 | forward 1 | Glyoxalase | Glyoxalase/Bleomycin resistance | 6.00E−38 |
| | | | | | | protein/Dioxygenase superfamily | |
| 112 | LG:1065433.1:2000SEP08 | 264 | 449 | forward 3 | KRAB | KRAB box | 1.30E−38 |
| 113 | LG:236992.4:2000SEP08 | 286 | 465 | forward 1 | Sema | Sema domain | 2.70E−21 |
| 113 | LG:236992.4:2000SEP08 | 209 | 292 | forward 2 | Sema | Sema domain | 3.60E−06 |
| 114 | LG:1071124.1:2000SEP08 | 469 | 630 | forward 1 | KRAB | KRAB box | 1.20E−16 |
| 115 | LG:206425.2:2000SEP08 | 109 | 519 | forward 1 | GBP | Guanylate-binding protein, N-terminal | 1.60E−100 |
| | | | | | | domain | |
| 115 | LG:206425.2:2000SEP08 | 491 | 916 | forward 2 | GBP | Guanylate-binding protein, N-terminal | 2.70E−70 |
| | | | | | | domain | |
| 115 | LG:206425.2:2000SEP08 | 1164 | 1685 | forward 3 | GBP_C | Guanylate-binding protein, C-terminal | 3.50E−71 |
| | | | | | | domain | |
| 115 | LG:206425.2:2000SEP08 | 920 | 1201 | forward 2 | GBP_C | Guanylate-binding protein, C-terminal | 3.10E−63 |
| | | | | | | domain | |
| 115 | LG:206425.2:2000SEP08 | 1651 | 1773 | forward 1 | GBP_C | Guanylate-binding protein, C-terminal | 1.80E−04 |
| | | | | | | domain | |
| 116 | LG:885747.2:2000SEP08 | 372 | 569 | forward 3 | Ribosomal_L31e | Ribosomal protein L31e | 9.50E−16 |
| 117 | LG:1140501.1:2000SEP08 | 176 | 340 | forward 2 | ATP1G1_PLM_{—} | ATP1G1/PLM/MAT8 family | 9.30E−22 |
| | | | | | MAT8 | | |
| 118 | LG:001239.1:2000SEP08 | 1686 | 1850 | forward 3 | ig | Immunoglobulin domain | 2.50E−08 |
| 118 | LG:001239.1:2000SEP08 | 1391 | 1561 | forward 2 | ig | Immunoglobulin domain | 4.00E−08 |
| 119 | LG:018980.1:2000SEP08 | 395 | 502 | forward 2 | GTP_EFTU | Elongation factor Tu family | 3.20E−09 |
| 119 | LG:018980.1:2000SEP08 | 634 | 801 | forward 1 | GTP_EFTU | Elongation factor Tu family | 3.50E−07 |
| 119 | LG:018980.1:2000SEP08 | 159 | 215 | forward 3 | GTP_EFTU | Elongation factor Tu family | 6.90E−07 |
| 120 | LG:1083120.3:2000SEP08 | 117 | 266 | forward 3 | KRAB | KRAB box | 5.10E−22 |
| 121 | LG:233258.3:2000SEP08 | 1797 | 2069 | forward 3 | cadherin | Cadherin domain | 3.90E−24 |
| 122 | LG:999062.1:2000SEP08 | 78 | 497 | forward 3 | Ribosomal_S19e | Ribosomal protein S19e | 2.10E−101 |
| 123 | LG:887776.1:2000SEP08 | 101 | 625 | forward 2 | transport_prot | Serum albumin family | 3.30E−92 |
| 123 | LG:887776.1:2000SEP08 | 699 | 1163 | forward 3 | transport_prot | Serum albumin family | 5.90E−41 |
| 124 | LG:1400301.2:2000SEP08 | 253 | 441 | forward 1 | KRAB | KRAB box | 2.10E−38 |
| 125 | LG:1329362.1:2000SEP08 | 194 | 262 | forward 2 | zf-C2H2 | Zinc finger, C2H2 type | 3.90E−06 |
| 126 | LG:1096498.1:2000SEP08 | 17 | 358 | forward 2 | 60s_ribosomal | 60s Acidic ribosomal protein | 1.70E−48 |
| 127 | LG:1096337.1:2000SEP08 | 512 | 637 | forward 2 | 60s_ribosomal | 60s Acidic ribosomal protein | 4.10E−07 |
| 127 | LG:1096337.1:2000SEP08 | 619 | 738 | forward 1 | 60s_ribosomal | 60s Acidic ribosomal protein | 1.50E−04 |
| 128 | LG:1400579.1:2000SEP08 | 200 | 268 | forward 2 | zf-C2H2 | Zinc finger, C2H2 type | 7.80E−05 |
| 129 | LG:1080091.1:2000SEP08 | 151 | 318 | forward 1 | KRAB | KRAB box | 1.80E−25 |
| 130 | LG:1082203.1:2000SEP08 | 961 | 1029 | forward 1 | zf-C2H2 | Zinc finger, C2H2 type | 2.10E−08 |
| 131 | LG:1084051.1:2000SEP08 | 195 | 263 | forward 3 | zf-C2H2 | Zinc finger, C2H2 type | 1.80E−06 |
| 132 | LG:1082393.1:2000SEP08 | 237 | 425 | forward 3 | KRAB | KRAB box | 1.80E−36 |
| 132 | LG:1082393.1:2000SEP08 | 1251 | 1319 | forward 3 | zf-C2H2 | Zinc finger, C2H2 type | 1.40E−06 |
| 133 | LG:1086183.1:2000SEP08 | 308 | 478 | forward 2 | KRAB | KRAB box | 1.50E−15 |
| 133 | LG:1086183.1:2000SEP08 | 833 | 901 | forward 2 | zf-C2H2 | Zinc finger, C2H2 type | 9.60E−07 |
| 134 | LG:1090268.1:2000SEP08 | 1167 | 1235 | forward 3 | zf-C2H2 | Zinc finger, C2H2 type | 1.00E−07 |
| 135 | LG:1400597.5:2000SEP08 | 63 | 131 | forward 3 | zf-C2H2 | Zinc finger, C2H2 type | 1.20E−04 |
| 136 | LG:1080307.2:2000SEP08 | 108 | 239 | forward 3 | KRAB | KRAB box | 3.60E−04 |
| 137 | LG:1400603.2:2000SEP08 | 299 | 490 | forward 2 | KRAB | KRAB box | 5.50E−41 |
| 137 | LG:1400603.2:2000SEP08 | 929 | 997 | forward 2 | zf-C2H2 | Zinc finger, C2H2 type | 1.20E−04 |
| 138 | LG:1052984.1:2000SEP08 | 142 | 330 | forward 1 | KRAB | KRAB box | 3.70E−41 |
| 139 | LG:1091259.1:2000SEP08 | 555 | 623 | forward 3 | zf-C2H2 | Zinc finger, C2H2 type | 1.20E−07 |
| 140 | LG:1082263.2:2000SEP08 | 258 | 443 | forward 3 | KRAB | KRAB box | 2.50E−29 |
| 140 | LG:1082263.2:2000SEP08 | 981 | 1049 | forward 3 | zf-C2H2 | Zinc finger, C2H2 type | 1.70E−07 |
| 141 | LG:1048604.2:2000SEP08 | 351 | 530 | forward 3 | KRAB | KRAB box | 3.50E−20 |
| 142 | LG:1085254.3:2000SEP08 | 746 | 814 | forward 2 | zf-C2H2 | Zinc finger, C2H2 type | 3.10E−07 |
| 143 | LG:1400606.2:2000SEP08 | 244 | 432 | forward 1 | KRAB | KRAB box | 7.60E−41 |
| 143 | LG:1400606.2:2000SEP08 | 904 | 972 | forward 1 | zf-C2H2 | Zinc finger, C2H2 type | 1.80E−07 |
| 144 | LG:1090358.2:2000SEP08 | 385 | 573 | forward 1 | KRAB | KRAB box | 1.30E−47 |
| 144 | LG:1090358.2:2000SEP08 | 862 | 930 | forward 1 | zf-C2H2 | Zinc finger, C2H2 type | 7.90E−07 |
| 145 | LG:1079064.2:2000SEP08 | 325 | 612 | forward 1 | SCAN | SCAN domain | 3.00E−39 |
| 146 | LG:1076866.1:2000SEP08 | 203 | 391 | forward 2 | KRAB | KRAB box | 6.20E−39 |
| 146 | LG:1076866.1:2000SEP08 | 1868 | 1936 | forward 2 | zf-C2H2 | Zinc finger, C2H2 type | 5.20E−08 |
| 147 | LG:969359.1:2000SEP08 | 221 | 715 | forward 2 | ferritin | Ferritin | 3.10E−86 |
| 148 | LG:366783.1:2000SEP08 | 70 | 609 | forward 1 | Zn_carbOpept | Zinc carboxypeptidase | 8.80E−76 |
| 148 | LG:366783.1:2000SEP08 | 705 | 806 | forward 3 | Zn_carbOpept | Zinc carboxypeptidase | 2.20E−10 |
| 148 | LG:366783.1:2000SEP08 | 623 | 724 | forward 2 | Zn_carbOpept | Zinc carboxypeptidase | 1.80E−05 |
| 149 | LG:332176.3:2000SEP08 | 5 | 1060 | forward 2 | Glyco_hydro_31 | Glycosyl hydrolases family 31 | 2.40E−163 |
| 150 | LG:994938.1:2000SEP08 | 3 | 95 | forward 3 | GBP | Guanylate-binding protein, N-terminal | 4.20E−14 |
| | | | | | | domain | |
| 151 | LG:982800.1:2000SEP08 | 16 | 243 | forward 1 | pkinase | Protein kinase domain | 1.30E−10 |
| 152 | LG:977850.7:2000SEP08 | 7 | 102 | forward 1 | zf-DHHC | DHHC zinc finger domain | 1.30E−04 |
| 153 | LG:234748.2:2000SEP08 | 6 | 149 | forward 3 | PTE | Phosphotriesterase family | 1.20E−19 |
| 154 | LG:306284.1:2000SEP08 | 197 | 613 | forward 2 | Band_41 | FERM domain (Band 4.1 family) | 2.00E−52 |
| 155 | LI:333170.3:2000SEP08 | 292 | 498 | forward 1 | HMG_box | HMG (high mobility group) box | 1.80E−22 |
| 156 | LI:336685.2:2000SEP08 | 913 | 1092 | forward 1 | homeobox | Homeobox domain | 4.20E−15 |
| 157 | LI:279013.5:2000SEP08 | 393 | 500 | forward 3 | WD40 | WD domain, G-beta repeat | 2.70E−05 |
| 158 | LI:1037075.1:2000SEP08 | 322 | 666 | forward 1 | ABC_tran | ABC transporter | 7.10E−04 |
| 158 | LI:1037075.1:2000SEP08 | 328 | 375 | forward 1 | PRK | Phosphoribulokinase/Uridine kinase | 7.40E−04 |
| | | | | | | family | |
| 159 | LI:1073403.1:2000SEP08 | 203 | 289 | forward 2 | efhand | EF hand | 5.50E−04 |
| 159 | LI:1073403.1:2000SEP08 | 56 | 187 | forward 2 | S_100 | S-100/CaBP type calcium binding | 3.60E−23 |
| | | | | | | domain | |
| 160 | LI:1075296.1:2000SEP08 | 337 | 543 | forward 1 | Ribosomal_L12 | Ribosomal protein L7/L12 C-terminal | 5.40E−09 |
| | | | | | | domain | |
| 161 | LI:1085501.1:2000SEP08 | 561 | 776 | forward 3 | EF1BD | EF-1 guanine nucleotide exchange | 1.20E−24 |
| | | | | | | domain | |
| 162 | LI:1086181.1:2000SEP08 | 305 | 469 | forward 2 | HTH_3 | Helix-turn-helix | 1.20E−10 |
| 163 | LI:1164493.1:2000SEP08 | 365 | 553 | forward 2 | KRAB | KRAB box | 8.10E−31 |
| 164 | LI:1175097.1:2000SEP08 | 460 | 648 | forward 1 | KRAB | KRAB box | 7.60E−41 |
| 165 | LI:1092948.1:2000SEP08 | 446 | 595 | forward 2 | KRAB | KRAB box | 2.80E−21 |
| 166 | LI:380378.2:2000SEP08 | 120 | 419 | forward 3 | mito_carr | Mitochondrial carrier protein | 1.30E−25 |
| 167 | LI:1029674.1:2000SEP08 | 360 | 431 | forward 3 | LRR | Leucine Rich Repeat | 9.00E−04 |
| 168 | LI:2048601.3:2000SEP08 | 2 | 145 | forward 2 | ig | Immunoglobulin domain | 3.00E−05 |
| 169 | LI:1186208.1:2000SEP08 | 161 | 349 | forward 2 | KRAB | KRAB box | 1.30E−40 |
| 170 | LI:1170753.1:2000SEP08 | 130 | 318 | forward 1 | KRAB | KRAB box | 8.40E−42 |
| 171 | LI:1180908.1:2000SEP08 | 509 | 577 | forward 2 | zf-C2H2 | Zinc finger, C2H2 type | 3.90E−06 |
| 171 | LI:1180908.1:2000SEP08 | 340 | 408 | forward 1 | zf-C2H2 | Zinc finger, C2H2 type | 1.30E−04 |
| 172 | LI:1182900.2:2000SEP08 | 346 | 414 | forward 1 | zf-C2H2 | Zinc finger, C2H2 type | 3.10E−07 |
| 173 | LI:1169548.2:2000SEP08 | 393 | 500 | forward 3 | VHP | Villin headpiece domain | 7.10E−20 |
| 174 | LI:1039974.1:2000SEP08 | 191 | 742 | forward 2 | pkinase | Protein kinase domain | 1.30E−37 |
| 175 | LI:1175765.2:2000SEP08 | 279 | 446 | forward 3 | KRAB | KRAB box | 5.20E−23 |
| 176 | LI:313948.1:2000SEP08 | 75 | 143 | forward 3 | zf-C2H2 | Zinc finger, C2H2 type | 2.20E−07 |
| 177 | LI:335923.2:2000SEP08 | 215 | 523 | forward 2 | lys | C-type lysozyme/alpha-lactalbumin | 1.60E−42 |
| | | | | | | family | |
| 178 | LI:345884.1:2000SEP08 | 208 | 360 | forward 1 | ig | Immunoglobulin domain | 7.40E−04 |
| 179 | LI:417127.1:2000SEP08 | 359 | 529 | forward 2 | KRAB | KRAB box | 4.90E−22 |
| 180 | LI:451710.1:2000SEP08 | 130 | 459 | forward 1 | Ribosomal_L32e | Ribosomal protein L32 | 4.80E−57 |
| 181 | LI:406882.2:2000SEP08 | 247 | 408 | forward 1 | Kelch | Kelch motif | 4.20E−11 |
| 181 | LI:406882.2:2000SEP08 | 656 | 793 | forward 2 | Kelch | Kelch motif | 8.90E−09 |
| 182 | LI:728223.1:2000SEP08 | 158 | 373 | forward 2 | rrm | RNA recognition motif. (a.k.a. RRM, | 1.20E−28 |
| | | | | | | RBD, or RNP domain) | |
| 183 | LI:289783.19:2000SEP08 | 821 | 1003 | forward 2 | thiored | Thioredoxin | 1.70E−20 |
| 183 | LI:289783.19:2000SEP08 | 747 | 800 | forward 3 | thiored | Thioredoxin | 5.10E−05 |
| 184 | LI:235255.8:2000SEP08 | 419 | 919 | forward 2 | TGT | Queuine tRNA-ribosyltransferase | 2.10E−24 |
| 184 | LI:235255.8:2000SEP08 | 747 | 1346 | forward 3 | TGT | Queuine tRNA-ribosyltransferase | 7.00E−15 |
| 185 | LI:237693.5:2000SEP08 | 48 | 518 | forward 3 | abhydrolase_2 | Phospholipase/Carboxylesterase | 6.20E−07 |
| 186 | LI:433670.3:2000SEP08 | 404 | 682 | forward 2 | Sema | Sema domain | 3.80E−26 |
| 187 | LI:202943.4:2000SEP08 | 119 | 223 | forward 2 | EGF | EGF-like domain | 1.60E−05 |
| 187 | LI:202943.4:2000SEP08 | 1304 | 1465 | forward 2 | sushi | Sushi domain (SCR repeat) | 3.80E−18 |
| 188 | LI:068682.1:2000SEP08 | 169 | 876 | forward 1 | pkinase | Protein kinase domain | 1.70E−65 |
| 189 | LI:203301.3:2000SEP08 | 570 | 716 | forward 3 | Band_41 | FERM domain (Band 4.1 family) | 1.50E−20 |
| 189 | LI:203301.3:2000SEP08 | 365 | 577 | forward 2 | Band_41 | FERM domain (Band 4.1 family) | 6.30E−16 |
| 190 | LI:020726.3:2000SEP08 | 542 | 1915 | forward 2 | MCT | Monocarboxylate transporter | 1.80E−98 |
| 191 | LI:027209.1:2000SEP08 | 379 | 987 | forward 1 | fibrinogen_C | Fibrinogen beta and gamma chains, C- | 7.80E−43 |
| | | | | | | terminal globular domain | |
| 192 | LI:108819.1:2000SEP08 | 316 | 828 | forward 1 | vwa | von Willebrand factor type A domain | 1.90E−52 |
| 193 | LI:021759.1:2000SEP08 | 1136 | 1246 | forward 2 | WD40 | WD domain, G-beta repeat | 1.70E−07 |
| 194 | LI:1165967.1:2000SEP08 | 322 | 462 | forward 1 | Ribosomal_S27 | Ribosomal protein S27a | 1.50E−30 |
| 194 | LI:1165967.1:2000SEP08 | 22 | 243 | forward 1 | ubiquitin | Ubiquitin family | 4.50E−42 |
| 195 | LI:1166315.1:2000SEP08 | 131 | 283 | forward 2 | pro_isomerase | Cyclophilin type peptidyl-prolyl cis- | 1.10E−26 |
| | | | | | | trans isomerase | |
| 195 | LI:1166315.1:2000SEP08 | 280 | 423 | forward 1 | pro_isomerase | Cyclophilin type peptidyl-prolyl cis- | 5.50E−17 |
| | | | | | | trans isomerase | |
| 195 | LI:1166315.1:2000SEP08 | 423 | 482 | forward 3 | pro_isomerase | Cyclophilin type peptidyl-prolyl cis- | 4.10E−06 |
| | | | | | | trans isomerase | |
| 196 | LI:204626.1:2000SEP08 | 322 | 1212 | forward 1 | Syntaxin | Syntaxin | 8.60E−44 |
| 197 | LI:801140.1:2000SEP08 | 516 | 584 | forward 3 | zf-C2H2 | Zinc finger, C2H2 type | 2.50E−08 |
| 198 | LI:286639.1:2000SEP08 | 1295 | 1714 | forward 2 | actin | Actin | 9.10E−64 |
| 198 | LI:286639.1:2000SEP08 | 772 | 1296 | forward 1 | actin | Actin | 1.60E−44 |
| 198 | LI:286639.1:2000SEP08 | 630 | 755 | forward 3 | actin | Actin | 1.10E−09 |
| 199 | LI:288905.4:2000SEP08 | 285 | 602 | forward 3 | CH | Calponin homology (CH) domain | 5.30E−27 |
| 200 | LI:332161.1:2000SEP08 | 75 | 710 | forward 3 | ras | Ras family | 2.00E−48 |
| 201 | LI:184867.1:2000SEP08 | 365 | 478 | forward 2 | ubiquitin | Ubiquitin family | 1.40E−04 |
| 202 | LI:229932.4:2000SEP08 | 58 | 969 | forward 1 | AMP-binding | AMP-binding enzyme | 3.00E−06 |
| 203 | LI:1189932.1:2000SEP08 | 95 | 1441 | forward 2 | Folate_carrier | Reduced folate carrier | 5.80E−10 |
| 204 | LI:1076689.1:2000SEP08 | 346 | 708 | forward 1 | ribonuclease_T2 | Ribonuclease T2 family | 6.50E−19 |
| 205 | LI:415181.2:2000SEP08 | 505 | 1584 | forward 1 | Inos-1-P_synth | Myo-inositol-1-phosphate synthase | 2.30E−136 |
| 205 | LI:415181.2:2000SEP08 | 294 | 1220 | forward 3 | Inos-1-P_synth | Myo-inositol-1-phosphate synthase | 3.40E−16 |
| 206 | LI:296358.1:2000SEP08 | 678 | 1061 | forward 3 | kinesin | Kinesin motor domain | 1.40E−46 |
| 206 | LI:296358.1:2000SEP08 | 163 | 510 | forward 1 | kinesin | Kinesin motor domain | 6.80E−24 |
| 207 | LI:205186.3:2000SEP08 | 476 | 982 | forward 2 | lactamase_B | Metallo-beta-lactamase superfamily | 3.70E−06 |
| 208 | LI:220537.2:2000SEP08 | 60 | 1574 | forward 3 | sugar_tr | Sugar (and other) transporter | 6.40E−07 |
| 209 | LI:248364.2:2000SEP08 | 253 | 594 | forward 1 | BTB | BTB/POZ domain | 3.60E−04 |
| 209 | LI:248364.2:2000SEP08 | 1544 | 1612 | forward 2 | zf-C2H2 | Zinc finger, C2H2 type | 7.10E−06 |
| 210 | LI:2048338.1:2000SEP08 | 261 | 410 | forward 3 | zf-C3HC4 | Zinc finger, C3HC4 type (RING finger) | 2.00E−07 |
| 211 | LI:1185203.8:2000SEP08 | 333 | 431 | forward 3 | ank | Ank repeat | 2.20E−07 |
| 212 | LI:021770.3:2000SEP08 | 287 | 1171 | forward 2 | adh_zinc | Zinc-binding dehydrogenases | 3.70E−08 |
| 213 | LI:1185841.1:2000SEP08 | 705 | 860 | forward 3 | myosin_head | Myosin head (motor domain) | 1.80E−07 |
| 213 | LI:1185841.1:2000SEP08 | 992 | 1063 | forward 2 | myosin_head | Myosin head (motor domain) | 3.20E−04 |
| 214 | LI:1181710.1:2000SEP08 | 62 | 130 | forward 2 | zf-C2H2 | Zinc finger, C2H2 type | 1.10E−05 |
| 215 | LI:2048959.1:2000SEP08 | 225 | 293 | forward 3 | zf-C2H2 | Zinc finger, C2H2 type | 4.10E−07 |
| 216 | LI:798494.1:2000SEP08 | 273 | 464 | forward 3 | KRAB | KRAB box | 2.00E−42 |
| 216 | LI:798494.1:2000SEP08 | 675 | 743 | forward 3 | zf-C2H2 | Zinc finger, C2H2 type | 4.50E−05 |
| 216 | LI:798494.1:2000SEP08 | 794 | 862 | forward 2 | zf-C2H2 | Zinc finger, C2H2 type | 6.80E−04 |
| 217 | LI:2049223.1:2000SEP08 | 130 | 318 | forward 1 | KRAB | KRAB box | 2.10E−42 |
| 218 | LI:1177833.1:2000SEP08 | 78 | 218 | forward 3 | KRAB | KRAB box | 2.30E−17 |
| 218 | LI:1177833.1:2000SEP08 | 999 | 1067 | forward 3 | zf-C2H2 | Zinc finger, C2H2 type | 1.70E−04 |
| 219 | LI:2049267.1:2000SEP08 | 78 | 227 | forward 3 | KRAB | KRAB box | 3.70E−22 |
| 220 | LI:1165939.1:2000SEP08 | 247 | 315 | forward 1 | zf-C2H2 | Zinc finger, C2H2 type | 2.60E−06 |
| 221 | LI:1170958.1:2000SEP08 | 397 | 465 | forward 1 | zf-C2H2 | Zinc finger, C2H2 type | 3.20E−07 |
| 222 | LI:1089827.1:2000SEP08 | 722 | 790 | forward 2 | zf-C2H2 | Zinc finger, C2H2 type | 3.10E−07 |
| 222 | LI:1089827.1:2000SEP08 | 105 | 173 | forward 3 | zf-C2H2 | Zinc finger, C2H2 type | 9.60E−07 |
| 222 | LI:1089827.1:2000SEP08 | 433 | 501 | forward 1 | zf-C2H2 | Zinc finger, C2H2 type | 2.10E−06 |
| 223 | LI:792112.1:2000SEP08 | 200 | 268 | forward 2 | zf-C2H2 | Zinc finger, C2H2 type | 7.80E−05 |
| 224 | LI:282219.2:2000SEP08 | 129 | 197 | forward 3 | zf-C2H2 | Zinc finger, C2H2 type | 4.10E−05 |
| 225 | LI:1088010.2:2000SEP08 | 294 | 362 | forward 3 | zf-C2H2 | Zinc finger, C2H2 type | 5.80E−08 |
| 226 | LI:1165276.1:2000SEP08 | 195 | 263 | forward 3 | zf-C2H2 | Zinc finger, C2H2 type | 1.80E−06 |
| 227 | LI:1169524.2:2000SEP08 | 267 | 455 | forward 3 | KRAB | KRAB box | 2.70E−40 |
| 227 | LI:1169524.2:2000SEP08 | 727 | 795 | forward 1 | zf-C2H2 | Zinc finger, C2H2 type | 7.90E−07 |
| 228 | LI:1180255.1:2000SEP08 | 440 | 628 | forward 2 | KRAB | KRAB box | 2.30E−33 |
| 228 | LI:1180255.1:2000SEP08 | 1027 | 1095 | forward 1 | zf-C2H2 | Zinc finger, C2H2 type | 2.50E−07 |
| 229 | LI:1091903.1:2000SEP08 | 182 | 370 | forward 2 | KRAB | KRAB box | 6.20E−39 |
| 230 | LI:1169219.1:2000SEP08 | 142 | 330 | forward 1 | KRAB | KRAB box | 3.70E−41 |
| 231 | LI:2050313.1:2000SEP08 | 803 | 982 | forward 2 | Collagen | Collagen triple helix repeat (20 copies) | 9.50E−10 |
| 232 | LI:209351.3:2000SEP08 | 561 | 671 | forward 3 | WD40 | WD domain, G-beta repeat | 2.20E−06 |
| 232 | LI:209351.3:2000SEP08 | 313 | 423 | forward 1 | WD40 | WD domain, G-beta repeat | 1.00E−04 |
| 233 | LI:119900.1:2000SEP08 | 179 | 358 | forward 2 | KRAB | KRAB box | 1.50E−34 |
| 234 | LI:2052274.1:2000SEP08 | 193 | 1197 | forward 1 | A_deaminase | Adenosine/AMP deaminase | 4.00E−10 |
| 235 | LI:1075502.1:2000SEP08 | 100 | 315 | forward 1 | OMPdecase | Orotidine 5′-phosphate decarboxylase | 6.30E−35 |
| 235 | LI:1075502.1:2000SEP08 | 305 | 451 | forward 2 | OMPdecase | Orotidine 5′-phosphate decarboxylase | 4.70E−26 |
| 235 | LI:1075502.1:2000SEP08 | 426 | 806 | forward 3 | OMPdecase | Orotidine 5′-phosphate decarboxylase | 1.80E−12 |
| 236 | LI:813697.1:2000SEP08 | 753 | 821 | forward 3 | zf-C2H2 | Zinc finger, C2H2 type | 1.60E−07 |
| 237 | LI:814261.1:2000SEP08 | 3 | 95 | forward 3 | GBP | Guanylate-binding protein, N-terminal | 4.20E−14 |
| | | | | | | domain | |
| 238 | LI:775334.1:2000SEP08 | 112 | 180 | forward 1 | zf-C2H2 | Zinc finger, C2H2 type | 2.10E−08 |
| 239 | LI:1180325.1:2000SEP08 | 495 | 563 | forward 3 | zf-C2H2 | Zinc finger, C2H2 type | 2.20E−07 |
| 240 | LI:1183147.3:2000SEP08 | 82 | 150 | forward 1 | zf-C2H2 | Zinc finger, C2H2 type | 1.20E−04 |
| 241 | LI:1175373.3:2000SEP08 | 191 | 379 | forward 2 | KRAB | KRAB box | 6.80E−45 |
| 242 | LI:813757.1:2000SEP08 | 268 | 453 | forward 1 | KRAB | KRAB box | 4.30E−38 |
| 243 | LI:1182979.2:2000SEP08 | 341 | 409 | forward 2 | zf-C2H2 | Zinc finger, C2H2 type | 3.60E−06 |
| 244 | LI:1177823.2:2000SEP08 | 1232 | 1372 | forward 2 | KRAB | KRAB box | 3.00E−15 |
| 245 | LI:1174279.1:2000SEP08 | 276 | 464 | forward 3 | KRAB | KRAB box | 1.40E−46 |
| 245 | LI:1174279.1:2000SEP08 | 753 | 821 | forward 3 | zf-C2H2 | Zinc finger, C2H2 type | 7.90E−07 |
| 246 | LI:1178411.1:2000SEP08 | 787 | 855 | forward 1 | zf-C2H2 | Zinc finger, C2H2 type | 1.20E−07 |
| 247 | LI:1182739.1:2000SEP08 | 582 | 650 | forward 3 | zf-C2H2 | Zinc finger, C2H2 type | 6.70E−08 |
| 248 | LI:234937.4:2000SEP08 | 61 | 477 | forward 1 | DSPc | Dual specificity phosphatase, catalytic | 8.70E−29 |
| | | | | | | domain | |
| 249 | LI:1170660.1:2000SEP08 | 35 | 103 | forward 2 | zf-C2H2 | Zinc finger, C2H2 type | 8.70E−07 |
| 250 | LI:1144409.1:2000SEP08 | 16 | 243 | forward 1 | pkinase | Protein kinase domain | 1.30E−10 |
| 251 | LI:246290.10:2000SEP08 | 256 | 453 | forward 1 | rrm | RNA recognition motif. (a.k.a. RRM, | 2.10E−08 |
| | | | | | | RBD, or RNP domain) | |
| 252 | LI:280034.1:2000SEP08 | 119 | 652 | forward 2 | AAA | ATPase family associated with various | 1.80E−64 |
| | | | | | | cellular activities (AAA) | |

TABLE 4

| SEQ ID NO: | Template ID | Start | Stop | Frame | Domain | Topology |
|---|---|---|---|---|---|---|
| 34 | LG:345884.1:2000SEP08 | 56 | 142 | forward 2 | TM | N out |
| 35 | LG:400967.1:2000SEP08 | 470 | 547 | forward 2 | TM | N out |
| 36 | LG:024556.6:2000SEP08 | 529 | 597 | forward 1 | TM | N in |
| 37 | LG:081189.3:2000SEP08 | 627 | 686 | forward 3 | TM | N out |
| 38 | LG:018258.1:2000SEP08 | 430 | 507 | forward 1 | TM | N out |
| 38 | LG:018258.1:2000SEP08 | 444 | 503 | forward 3 | TM | N out |
| 39 | LG:450399.3:2000SEP08 | 145 | 231 | forward 1 | TM | N out |
| 39 | LG:450399.3:2000SEP08 | 417 | 503 | forward 3 | TM | N in |
| 40 | LG:451122.1:2000SEP08 | 328 | 390 | forward 1 | TM | N in |
| 41 | LG:451682.1:2000SEP08 | 93 | 155 | forward 3 | TM | |
| 42 | LG:238631.4:2000SEP08 | 526 | 612 | forward 1 | TM | N out |
| 42 | LG:238631.4:2000SEP08 | 477 | 563 | forward 3 | TM | |
| 43 | LG:236654.1:2000SEP08 | 236 | 295 | forward 2 | TM | N out |
| 44 | LG:332655.1:2000SEP08 | 541 | 621 | forward 1 | TM | N out |
| 44 | LG:332655.1:2000SEP08 | 536 | 598 | forward 2 | TM | N out |
| 44 | LG:332655.1:2000SEP08 | 626 | 688 | forward 2 | TM | N out |
| 44 | LG:332655.1:2000SEP08 | 887 | 949 | forward 2 | TM | N out |
| 44 | LG:332655.1:2000SEP08 | 965 | 1027 | forward 2 | TM | N out |
| 44 | LG:332655.1:2000SEP08 | 912 | 998 | forward 3 | TM | N out |
| 45 | LG:217396.2:2000SEP08 | 317 | 391 | forward 2 | TM | N out |
| 45 | LG:217396.2:2000SEP08 | 1127 | 1189 | forward 2 | TM | N out |
| 46 | LG:090574.1:2000SEP08 | 49 | 108 | forward 1 | TM | N out |
| 47 | LG:202943.1:2000SEP08 | 590 | 658 | forward 2 | TM | N out |
| 47 | LG:202943.1:2000SEP08 | 842 | 928 | forward 2 | TM | N out |
| 47 | LG:202943.1:2000SEP08 | 552 | 638 | forward 3 | TM | N out |
| 48 | LG:236928.1:2000SEP08 | 349 | 435 | forward 1 | TM | N in |
| 48 | LG:236928.1:2000SEP08 | 448 | 519 | forward 1 | TM | N in |
| 48 | LG:236928.1:2000SEP08 | 592 | 642 | forward 1 | TM | N in |
| 48 | LG:236928.1:2000SEP08 | 706 | 768 | forward 1 | TM | N in |
| 48 | LG:236928.1:2000SEP08 | 778 | 840 | forward 1 | TM | N in |
| 48 | LG:236928.1:2000SEP08 | 967 | 1038 | forward 1 | TM | N in |
| 48 | LG:236928.1:2000SEP08 | 1318 | 1374 | forward 1 | TM | N in |
| 48 | LG:236928.1:2000SEP08 | 1609 | 1695 | forward 1 | TM | N in |
| 48 | LG:236928.1:2000SEP08 | 1933 | 2016 | forward 1 | TM | N in |
| 48 | LG:236928.1:2000SEP08 | 2161 | 2247 | forward 1 | TM | N in |
| 48 | LG:236928.1:2000SEP08 | 2419 | 2475 | forward 1 | TM | N in |
| 48 | LG:236928.1:2000SEP08 | 2818 | 2865 | forward 1 | TM | N in |
| 48 | LG:236928.1:2000SEP08 | 2884 | 2946 | forward 1 | TM | N in |
| 48 | LG:236928.1:2000SEP08 | 2980 | 3042 | forward 1 | TM | N in |
| 48 | LG:236928.1:2000SEP08 | 3076 | 3138 | forward 1 | TM | N in |
| 48 | LG:236928.1:2000SEP08 | 335 | 421 | forward 2 | TM | N in |
| 48 | LG:236928.1:2000SEP08 | 437 | 499 | forward 2 | TM | N in |
| 48 | LG:236928.1:2000SEP08 | 524 | 586 | forward 2 | TM | N in |
| 48 | LG:236928.1:2000SEP08 | 659 | 718 | forward 2 | TM | N in |
| 48 | LG:236928.1:2000SEP08 | 1268 | 1351 | forward 2 | TM | N in |
| 48 | LG:236928.1:2000SEP08 | 1400 | 1471 | forward 2 | TM | N in |
| 48 | LG:236928.1:2000SEP08 | 1472 | 1534 | forward 2 | TM | N in |
| 48 | LG:236928.1:2000SEP08 | 1586 | 1669 | forward 2 | TM | N in |
| 48 | LG:236928.1:2000SEP08 | 1745 | 1831 | forward 2 | TM | N in |
| 48 | LG:236928.1:2000SEP08 | 1877 | 1963 | forward 2 | TM | N in |
| 48 | LG:236928.1:2000SEP08 | 2015 | 2101 | forward 2 | TM | N in |
| 48 | LG:236928.1:2000SEP08 | 2501 | 2587 | forward 2 | TM | N in |
| 48 | LG:236928.1:2000SEP08 | 2639 | 2716 | forward 2 | TM | N in |
| 48 | LG:236928.1:2000SEP08 | 2786 | 2872 | forward 2 | TM | N in |
| 48 | LG:236928.1:2000SEP08 | 2894 | 2980 | forward 2 | TM | N in |
| 48 | LG:236928.1:2000SEP08 | 471 | 551 | forward 3 | TM | N in |
| 48 | LG:236928.1:2000SEP08 | 738 | 824 | forward 3 | TM | N in |
| 48 | LG:236928.1:2000SEP08 | 1287 | 1340 | forward 3 | TM | N in |
| 48 | LG:236928.1:2000SEP08 | 1626 | 1712 | forward 3 | TM | N in |
| 48 | LG:236928.1:2000SEP08 | 1890 | 1976 | forward 3 | TM | N in |
| 48 | LG:236928.1:2000SEP08 | 2034 | 2120 | forward 3 | TM | N in |
| 48 | LG:236928.1:2000SEP08 | 2118 | 2186 | forward 3 | TM | N in |
| 48 | LG:236928.1:2000SEP08 | 2520 | 2591 | forward 3 | TM | N in |
| 48 | LG:236928.1:2000SEP08 | 2763 | 2846 | forward 3 | TM | N in |
| 48 | LG:236928.1:2000SEP08 | 2916 | 2978 | forward 3 | TM | N in |
| 48 | LG:236928.1:2000SEP08 | 3003 | 3065 | forward 3 | TM | N in |
| 49 | LG:215169.2:2000SEP08 | 1363 | 1413 | forward 1 | TM | N in |
| 49 | LG:215169.2:2000SEP08 | 503 | 589 | forward 2 | TM | N out |
| 49 | LG:215169.2:2000SEP08 | 1727 | 1813 | forward 2 | TM | N out |
| 49 | LG:215169.2:2000SEP08 | 237 | 302 | forward 3 | TM | N out |
| 49 | LG:215169.2:2000SEP08 | 837 | 923 | forward 3 | TM | N out |
| 49 | LG:215169.2:2000SEP08 | 1398 | 1460 | forward 3 | TM | N out |
| 49 | LG:215169.2:2000SEP08 | 1470 | 1532 | forward 3 | TM | N out |
| 49 | LG:215169.2:2000SEP08 | 1605 | 1658 | forward 3 | TM | N out |
| 50 | LG:410726.1:2000SEP08 | 31 | 93 | forward 1 | TM | N out |
| 50 | LG:410726.1:2000SEP08 | 115 | 177 | forward 1 | TM | N out |
| 50 | LG:410726.1:2000SEP08 | 463 | 549 | forward 1 | TM | N out |
| 51 | LG:234372.2:2000SEP08 | 325 | 411 | forward 1 | TM | N in |
| 51 | LG:234372.2:2000SEP08 | 2492 | 2578 | forward 2 | TM | N out |
| 51 | LG:234372.2:2000SEP08 | 2669 | 2743 | forward 2 | TM | N out |
| 51 | LG:234372.2:2000SEP08 | 723 | 809 | forward 3 | TM | N in |
| 52 | LG:022629.1:2000SEP08 | 268 | 354 | forward 1 | TM | N out |
| 52 | LG:022629.1:2000SEP08 | 385 | 459 | forward 1 | TM | N out |
| 52 | LG:022629.1:2000SEP08 | 559 | 645 | forward 1 | TM | N out |
| 52 | LG:022629.1:2000SEP08 | 796 | 882 | forward 1 | TM | N out |
| 52 | LG:022629.1:2000SEP08 | 281 | 358 | forward 2 | TM | N in |
| 53 | LG:068682.1:2000SEP08 | 707 | 793 | forward 2 | TM | N out |
| 54 | LG:222335.1:2000SEP08 | 847 | 933 | forward 1 | TM | N in |
| 54 | LG:222335.1:2000SEP08 | 973 | 1023 | forward 1 | TM | N in |
| 54 | LG:222335.1:2000SEP08 | 1030 | 1101 | forward 1 | TM | N in |
| 54 | LG:222335.1:2000SEP08 | 1216 | 1290 | forward 1 | TM | N in |
| 54 | LG:222335.1:2000SEP08 | 23 | 109 | forward 2 | TM | |
| 54 | LG:222335.1:2000SEP08 | 314 | 373 | forward 2 | TM | |
| 54 | LG:222335.1:2000SEP08 | 428 | 514 | forward 2 | TM | |
| 54 | LG:222335.1:2000SEP08 | 728 | 784 | forward 2 | TM | |
| 54 | LG:222335.1:2000SEP08 | 794 | 868 | forward 2 | TM | |
| 54 | LG:222335.1:2000SEP08 | 965 | 1051 | forward 2 | TM | |
| 54 | LG:222335.1:2000SEP08 | 603 | 674 | forward 3 | TM | N in |
| 54 | LG:222335.1:2000SEP08 | 921 | 983 | forward 3 | TM | N in |
| 54 | LG:222335.1:2000SEP08 | 1014 | 1076 | forward 3 | TM | N in |
| 54 | LG:222335.1:2000SEP08 | 1101 | 1187 | forward 3 | TM | N in |
| 54 | LG:222335.1:2000SEP08 | 1227 | 1277 | forward 3 | TM | N in |
| 55 | LG:331342.1:2000SEP08 | 22 | 96 | forward 1 | TM | N out |
| 55 | LG:331342.1:2000SEP08 | 289 | 339 | forward 1 | TM | N out |
| 55 | LG:331342.1:2000SEP08 | 645 | 731 | forward 3 | TM | N in |
| 56 | LG:021770.1:2000SEP08 | 286 | 372 | forward 1 | TM | N in |
| 56 | LG:021770.1:2000SEP08 | 400 | 477 | forward 1 | TM | N in |
| 56 | LG:021770.1:2000SEP08 | 502 | 579 | forward 1 | TM | N in |
| 56 | LG:021770.1:2000SEP08 | 784 | 852 | forward 1 | TM | N in |
| 56 | LG:021770.1:2000SEP08 | 1237 | 1323 | forward 1 | TM | N in |
| 56 | LG:021770.1:2000SEP08 | 2416 | 2499 | forward 1 | TM | N in |
| 56 | LG:021770.1:2000SEP08 | 2611 | 2697 | forward 1 | TM | N in |
| 56 | LG:021770.1:2000SEP08 | 1115 | 1201 | forward 2 | TM | N in |
| 56 | LG:021770.1:2000SEP08 | 1262 | 1336 | forward 2 | TM | N in |
| 56 | LG:021770.1:2000SEP08 | 2426 | 2509 | forward 2 | TM | N in |
| 56 | LG:021770.1:2000SEP08 | 2726 | 2776 | forward 2 | TM | N in |
| 56 | LG:021770.1:2000SEP08 | 534 | 614 | forward 3 | TM | N in |
| 56 | LG:021770.1:2000SEP08 | 1260 | 1346 | forward 3 | TM | N in |
| 56 | LG:021770.1:2000SEP08 | 1461 | 1547 | forward 3 | TM | N in |
| 56 | LG:021770.1:2000SEP08 | 1695 | 1778 | forward 3 | TM | N in |
| 56 | LG:021770.1:2000SEP08 | 2496 | 2576 | forward 3 | TM | N in |
| 56 | LG:021770.1:2000SEP08 | 2727 | 2792 | forward 3 | TM | N in |
| 57 | LG:181607.9:2000SEP08 | 439 | 525 | forward 1 | TM | N out |
| 58 | LG:1042768.1:2000SEP08 | 695 | 751 | forward 2 | TM | N out |
| 58 | LG:1042768.1:2000SEP08 | 93 | 143 | forward 3 | TM | N in |
| 58 | LG:1042768.1:2000SEP08 | 201 | 287 | forward 3 | TM | N in |
| 58 | LG:1042768.1:2000SEP08 | 351 | 437 | forward 3 | TM | N in |
| 59 | LG:282729.1:2000SEP08 | 256 | 309 | forward 1 | TM | N out |
| 60 | LG:998305.3:2000SEP08 | 429 | 488 | forward 3 | TM | N in |
| 60 | LG:998305.3:2000SEP08 | 705 | 770 | forward 3 | TM | N in |
| 61 | LG:1135213.1:2000SEP08 | 41 | 127 | forward 2 | TM | N out |
| 61 | LG:1135213.1:2000SEP08 | 215 | 274 | forward 2 | TM | N out |
| 61 | LG:1135213.1:2000SEP08 | 293 | 379 | forward 2 | TM | N out |
| 61 | LG:1135213.1:2000SEP08 | 389 | 475 | forward 2 | TM | N out |
| 62 | LG:267762.1:2000SEP08 | 854 | 937 | forward 2 | TM | N in |
| 62 | LG:267762.1:2000SEP08 | 1283 | 1345 | forward 2 | TM | N in |
| 62 | LG:267762.1:2000SEP08 | 1445 | 1531 | forward 2 | TM | N in |
| 62 | LG:267762.1:2000SEP08 | 1383 | 1469 | forward 3 | TM | N out |
| 63 | LG:120744.1:2000SEP08 | 181 | 249 | forward 1 | TM | N out |
| 63 | LG:120744.1:2000SEP08 | 188 | 256 | forward 2 | TM | |
| 63 | LG:120744.1:2000SEP08 | 275 | 328 | forward 2 | TM | |
| 64 | LG:403409.1:2000SEP08 | 136 | 222 | forward 1 | TM | N out |
| 64 | LG:403409.1:2000SEP08 | 973 | 1029 | forward 1 | TM | N out |
| 64 | LG:403409.1:2000SEP08 | 1285 | 1371 | forward 1 | TM | N out |
| 64 | LG:403409.1:2000SEP08 | 182 | 268 | forward 2 | TM | N in |
| 65 | LG:226874.3:2000SEP08 | 231 | 278 | forward 3 | TM | N out |
| 66 | LG:1045521.4:2000SEP08 | 1216 | 1266 | forward 1 | TM | N in |
| 66 | LG:1045521.4:2000SEP08 | 1567 | 1653 | forward 1 | TM | N in |
| 66 | LG:1045521.4:2000SEP08 | 1699 | 1761 | forward 1 | TM | N in |
| 66 | LG:1045521.4:2000SEP08 | 3091 | 3177 | forward 1 | TM | N in |
| 66 | LG:1045521.4:2000SEP08 | 3508 | 3582 | forward 1 | TM | N in |
| 66 | LG:1045521.4:2000SEP08 | 1652 | 1714 | forward 2 | TM | N in |
| 66 | LG:1045521.4:2000SEP08 | 2444 | 2500 | forward 2 | TM | N in |
| 66 | LG:1045521.4:2000SEP08 | 2627 | 2713 | forward 2 | TM | N in |
| 66 | LG:1045521.4:2000SEP08 | 3017 | 3088 | forward 2 | TM | N in |
| 66 | LG:1045521.4:2000SEP08 | 3317 | 3385 | forward 2 | TM | N in |
| 66 | LG:1045521.4:2000SEP08 | 1530 | 1604 | forward 3 | TM | N in |
| 66 | LG:1045521.4:2000SEP08 | 2496 | 2549 | forward 3 | TM | N in |
| 66 | LG:1045521.4:2000SEP08 | 2931 | 3017 | forward 3 | TM | N in |
| 66 | LG:1045521.4:2000SEP08 | 3267 | 3341 | forward 3 | TM | N in |
| 67 | LG:275876.1:2000SEP08 | 775 | 849 | forward 1 | TM | N in |
| 67 | LG:275876.1:2000SEP08 | 949 | 1002 | forward 1 | TM | N in |
| 67 | LG:275876.1:2000SEP08 | 842 | 928 | forward 2 | TM | N out |
| 67 | LG:275876.1:2000SEP08 | 777 | 842 | forward 3 | TM | N in |
| 68 | LG:475127.7:2000SEP08 | 137 | 223 | forward 2 | TM | N in |
| 69 | LG:157263.1:2000SEP08 | 175 | 249 | forward 1 | TM | N in |
| 69 | LG:157263.1:2000SEP08 | 295 | 345 | forward 1 | TM | N in |
| 69 | LG:157263.1:2000SEP08 | 406 | 492 | forward 1 | TM | N in |
| 69 | LG:157263.1:2000SEP08 | 793 | 867 | forward 1 | TM | N in |
| 69 | LG:157263.1:2000SEP08 | 889 | 951 | forward 1 | TM | N in |
| 69 | LG:157263.1:2000SEP08 | 1081 | 1143 | forward 1 | TM | N in |
| 69 | LG:157263.1:2000SEP08 | 1168 | 1230 | forward 1 | TM | N in |
| 69 | LG:157263.1:2000SEP08 | 1255 | 1341 | forward 1 | TM | N in |
| 69 | LG:157263.1:2000SEP08 | 482 | 544 | forward 2 | TM | N out |
| 69 | LG:157263.1:2000SEP08 | 563 | 625 | forward 2 | TM | N out |
| 69 | LG:157263.1:2000SEP08 | 180 | 266 | forward 3 | TM | N out |
| 70 | LG:247382.7:2000SEP08 | 808 | 894 | forward 1 | TM | |
| 70 | LG:247382.7:2000SEP08 | 650 | 709 | forward 2 | TM | N out |
| 70 | LG:247382.7:2000SEP08 | 1412 | 1495 | forward 2 | TM | N out |
| 71 | LG:197367.5:2000SEP08 | 454 | 510 | forward 1 | TM | N out |
| 72 | LG:218090.5:2000SEP08 | 85 | 156 | forward 1 | TM | N out |
| 72 | LG:218090.5:2000SEP08 | 417 | 494 | forward 3 | TM | N out |
| 73 | LG:216612.4:2000SEP08 | 1615 | 1701 | forward 1 | TM | N in |
| 73 | LG:216612.4:2000SEP08 | 1942 | 2007 | forward 1 | TM | N in |
| 73 | LG:216612.4:2000SEP08 | 2182 | 2268 | forward 1 | TM | N in |
| 73 | LG:216612.4:2000SEP08 | 2413 | 2487 | forward 1 | TM | N in |
| 73 | LG:216612.4:2000SEP08 | 1583 | 1660 | forward 2 | TM | N in |
| 73 | LG:216612.4:2000SEP08 | 2114 | 2176 | forward 2 | TM | N in |
| 73 | LG:216612.4:2000SEP08 | 2216 | 2278 | forward 2 | TM | N in |
| 73 | LG:216612.4:2000SEP08 | 2339 | 2425 | forward 2 | TM | N in |
| 73 | LG:216612.4:2000SEP08 | 2483 | 2542 | forward 2 | TM | N in |
| 73 | LG:216612.4:2000SEP08 | 120 | 206 | forward 3 | TM | N in |
| 73 | LG:216612.4:2000SEP08 | 234 | 284 | forward 3 | TM | N in |
| 73 | LG:216612.4:2000SEP08 | 327 | 413 | forward 3 | TM | N in |
| 73 | LG:216612.4:2000SEP08 | 444 | 530 | forward 3 | TM | N in |
| 73 | LG:216612.4:2000SEP08 | 810 | 896 | forward 3 | TM | N in |
| 73 | LG:216612.4:2000SEP08 | 942 | 1028 | forward 3 | TM | N in |
| 73 | LG:216612.4:2000SEP08 | 1644 | 1730 | forward 3 | TM | N in |
| 73 | LG:216612.4:2000SEP08 | 1950 | 2012 | forward 3 | TM | N in |
| 73 | LG:216612.4:2000SEP08 | 2037 | 2099 | forward 3 | TM | N in |
| 73 | LG:216612.4:2000SEP08 | 2292 | 2378 | forward 3 | TM | N in |
| 73 | LG:216612.4:2000SEP08 | 2529 | 2615 | forward 3 | TM | N in |
| 74 | LG:197614.1:2000SEP08 | 484 | 537 | forward 1 | TM | N in |
| 74 | LG:197614.1:2000SEP08 | 2308 | 2367 | forward 1 | TM | N in |
| 74 | LG:197614.1:2000SEP08 | 3175 | 3246 | forward 1 | TM | N in |
| 74 | LG:197614.1:2000SEP08 | 3298 | 3354 | forward 1 | TM | N in |
| 74 | LG:197614.1:2000SEP08 | 3556 | 3612 | forward 1 | TM | N in |
| 74 | LG:197614.1:2000SEP08 | 3739 | 3825 | forward 1 | TM | N in |
| 74 | LG:197614.1:2000SEP08 | 3964 | 4050 | forward 1 | TM | N in |
| 74 | LG:197614.1:2000SEP08 | 4063 | 4134 | forward 1 | TM | N in |
| 74 | LG:197614.1:2000SEP08 | 4147 | 4215 | forward 1 | TM | N in |
| 74 | LG:197614.1:2000SEP08 | 374 | 460 | forward 2 | TM | N out |
| 74 | LG:197614.1:2000SEP08 | 800 | 862 | forward 2 | TM | N out |
| 74 | LG:197614.1:2000SEP08 | 875 | 937 | forward 2 | TM | N out |
| 74 | LG:197614.1:2000SEP08 | 1352 | 1414 | forward 2 | TM | N out |
| 74 | LG:197614.1:2000SEP08 | 1436 | 1498 | forward 2 | TM | N out |
| 74 | LG:197614.1:2000SEP08 | 2312 | 2398 | forward 2 | TM | N out |
| 74 | LG:197614.1:2000SEP08 | 3422 | 3469 | forward 2 | TM | N out |
| 74 | LG:197614.1:2000SEP08 | 3632 | 3718 | forward 2 | TM | N out |
| 74 | LG:197614.1:2000SEP08 | 3773 | 3835 | forward 2 | TM | N out |
| 74 | LG:197614.1:2000SEP08 | 3932 | 4003 | forward 2 | TM | N out |
| 74 | LG:197614.1:2000SEP08 | 4040 | 4126 | forward 2 | TM | N out |
| 74 | LG:197614.1:2000SEP08 | 4244 | 4312 | forward 2 | TM | N out |
| 74 | LG:197614.1:2000SEP08 | 4448 | 4504 | forward 2 | TM | N out |
| 74 | LG:197614.1:2000SEP08 | 489 | 575 | forward 3 | TM | |
| 74 | LG:197614.1:2000SEP08 | 2967 | 3017 | forward 3 | TM | |
| 74 | LG:197614.1:2000SEP08 | 3663 | 3749 | forward 3 | TM | |
| 74 | LG:197614.1:2000SEP08 | 3912 | 3974 | forward 3 | TM | |
| 74 | LG:197614.1:2000SEP08 | 4020 | 4082 | forward 3 | TM | |
| 74 | LG:197614.1:2000SEP08 | 4341 | 4427 | forward 3 | TM | |
| 75 | LG:378428.1:2000SEP08 | 703 | 789 | forward 1 | TM | N in |
| 75 | LG:378428.1:2000SEP08 | 1336 | 1422 | forward 1 | TM | N in |
| 75 | LG:378428.1:2000SEP08 | 1924 | 2010 | forward 1 | TM | N in |
| 75 | LG:378428.1:2000SEP08 | 2188 | 2268 | forward 1 | TM | N in |
| 75 | LG:378428.1:2000SEP08 | 2171 | 2224 | forward 2 | TM | N in |
| 75 | LG:378428.1:2000SEP08 | 1527 | 1577 | forward 3 | TM | N out |
| 75 | LG:378428.1:2000SEP08 | 1665 | 1751 | forward 3 | TM | N out |
| 75 | LG:378428.1:2000SEP08 | 1968 | 2054 | forward 3 | TM | N out |
| 75 | LG:378428.1:2000SEP08 | 2205 | 2291 | forward 3 | TM | N out |
| 76 | LG:286639.1:2000SEP08 | 127 | 213 | forward 1 | TM | |
| 76 | LG:286639.1:2000SEP08 | 970 | 1032 | forward 1 | TM | |
| 76 | LG:286639.1:2000SEP08 | 1066 | 1128 | forward 1 | TM | |
| 76 | LG:286639.1:2000SEP08 | 1588 | 1662 | forward 1 | TM | |
| 76 | LG:286639.1:2000SEP08 | 1786 | 1872 | forward 1 | TM | |
| 76 | LG:286639.1:2000SEP08 | 62 | 148 | forward 2 | TM | N in |
| 76 | LG:286639.1:2000SEP08 | 1772 | 1825 | forward 2 | TM | N in |
| 76 | LG:286639.1:2000SEP08 | 267 | 320 | forward 3 | TM | N out |
| 76 | LG:286639.1:2000SEP08 | 999 | 1085 | forward 3 | TM | N out |
| 76 | LG:286639.1:2000SEP08 | 1902 | 1967 | forward 3 | TM | N out |
| 77 | LG:389870.1:2000SEP08 | 1066 | 1113 | forward 1 | TM | N out |
| 77 | LG:389870.1:2000SEP08 | 1055 | 1120 | forward 2 | TM | N in |
| 77 | LG:389870.1:2000SEP08 | 1059 | 1142 | forward 3 | TM | N in |
| 78 | LG:1387485.6:2000SEP08 | 1213 | 1275 | forward 1 | TM | N in |
| 78 | LG:1387485.6:2000SEP08 | 206 | 292 | forward 2 | TM | N in |
| 78 | LG:1387485.6:2000SEP08 | 824 | 886 | forward 2 | TM | N in |
| 78 | LG:1387485.6:2000SEP08 | 914 | 976 | forward 2 | TM | N in |
| 78 | LG:1387485.6:2000SEP08 | 1244 | 1321 | forward 2 | TM | N in |
| 78 | LG:1387485.6:2000SEP08 | 396 | 473 | forward 3 | TM | N out |
| 78 | LG:1387485.6:2000SEP08 | 537 | 602 | forward 3 | TM | N out |
| 78 | LG:1387485.6:2000SEP08 | 660 | 746 | forward 3 | TM | N out |
| 78 | LG:1387485.6:2000SEP08 | 786 | 872 | forward 3 | TM | N out |
| 78 | LG:1387485.6:2000SEP08 | 1164 | 1226 | forward 3 | TM | N out |
| 78 | LG:1387485.6:2000SEP08 | 1245 | 1307 | forward 3 | TM | N out |
| 79 | LG:230151.1:2000SEP08 | 1774 | 1860 | forward 1 | TM | N in |
| 79 | LG:230151.1:2000SEP08 | 1091 | 1165 | forward 2 | TM | N out |
| 79 | LG:230151.1:2000SEP08 | 1193 | 1246 | forward 2 | TM | N out |
| 79 | LG:230151.1:2000SEP08 | 1757 | 1843 | forward 2 | TM | N out |
| 80 | LG:215158.5:2000SEP08 | 199 | 267 | forward 1 | TM | N out |
| 80 | LG:215158.5:2000SEP08 | 820 | 873 | forward 1 | TM | N out |
| 80 | LG:215158.5:2000SEP08 | 892 | 945 | forward 1 | TM | N out |
| 80 | LG:215158.5:2000SEP08 | 908 | 985 | forward 2 | TM | N out |
| 80 | LG:215158.5:2000SEP08 | 1433 | 1504 | forward 2 | TM | N out |
| 80 | LG:215158.5:2000SEP08 | 447 | 533 | forward 3 | TM | |
| 81 | LG:235840.1:2000SEP08 | 265 | 342 | forward 1 | TM | N out |
| 81 | LG:235840.1:2000SEP08 | 511 | 579 | forward 1 | TM | N out |
| 81 | LG:235840.1:2000SEP08 | 577 | 639 | forward 1 | TM | N out |
| 81 | LG:235840.1:2000SEP08 | 730 | 786 | forward 1 | TM | N out |
| 81 | LG:235840.1:2000SEP08 | 2011 | 2082 | forward 1 | TM | N out |
| 81 | LG:235840.1:2000SEP08 | 2110 | 2172 | forward 1 | TM | N out |
| 81 | LG:235840.1:2000SEP08 | 2326 | 2406 | forward 1 | TM | N out |
| 81 | LG:235840.1:2000SEP08 | 2416 | 2502 | forward 1 | TM | N out |
| 81 | LG:235840.1:2000SEP08 | 116 | 178 | forward 2 | TM | N out |
| 81 | LG:235840.1:2000SEP08 | 191 | 253 | forward 2 | TM | N out |
| 81 | LG:235840.1:2000SEP08 | 500 | 586 | forward 2 | TM | N out |
| 81 | LG:235840.1:2000SEP08 | 731 | 817 | forward 2 | TM | N out |
| 81 | LG:235840.1:2000SEP08 | 848 | 916 | forward 2 | TM | N out |
| 81 | LG:235840.1:2000SEP08 | 956 | 1030 | forward 2 | TM | N out |
| 81 | LG:235840.1:2000SEP08 | 1973 | 2050 | forward 2 | TM | N out |
| 81 | LG:235840.1:2000SEP08 | 192 | 278 | forward 3 | TM | N out |
| 81 | LG:235840.1:2000SEP08 | 516 | 590 | forward 3 | TM | N out |
| 81 | LG:235840.1:2000SEP08 | 711 | 797 | forward 3 | TM | N out |
| 81 | LG:235840.1:2000SEP08 | 819 | 905 | forward 3 | TM | N out |
| 81 | LG:235840.1:2000SEP08 | 1446 | 1529 | forward 3 | TM | N out |
| 81 | LG:235840.1:2000SEP08 | 1893 | 1976 | forward 3 | TM | N out |
| 81 | LG:235840.1:2000SEP08 | 2298 | 2360 | forward 3 | TM | N out |
| 82 | LG:350272.1:2000SEP08 | 1675 | 1746 | forward 1 | TM | N in |
| 82 | LG:350272.1:2000SEP08 | 2374 | 2436 | forward 1 | TM | N in |
| 82 | LG:350272.1:2000SEP08 | 104 | 190 | forward 2 | TM | N in |
| 82 | LG:350272.1:2000SEP08 | 2264 | 2332 | forward 2 | TM | N in |
| 82 | LG:350272.1:2000SEP08 | 2369 | 2440 | forward 2 | TM | N in |
| 82 | LG:350272.1:2000SEP08 | 12 | 95 | forward 3 | TM | N in |
| 82 | LG:350272.1:2000SEP08 | 993 | 1052 | forward 3 | TM | N in |
| 82 | LG:350272.1:2000SEP08 | 2067 | 2138 | forward 3 | TM | N in |
| 82 | LG:350272.1:2000SEP08 | 2334 | 2399 | forward 3 | TM | N in |
| 83 | LG:232190.1:2000SEP08 | 592 | 678 | forward 1 | TM | N in |
| 83 | LG:232190.1:2000SEP08 | 1423 | 1500 | forward 1 | TM | N in |
| 83 | LG:232190.1:2000SEP08 | 158 | 241 | forward 2 | TM | N in |
| 83 | LG:232190.1:2000SEP08 | 293 | 373 | forward 2 | TM | N in |
| 83 | LG:232190.1:2000SEP08 | 644 | 730 | forward 2 | TM | N in |
| 83 | LG:232190.1:2000SEP08 | 761 | 847 | forward 2 | TM | N in |
| 83 | LG:232190.1:2000SEP08 | 1466 | 1552 | forward 2 | TM | N in |
| 83 | LG:232190.1:2000SEP08 | 279 | 356 | forward 3 | TM | N in |
| 83 | LG:232190.1:2000SEP08 | 588 | 674 | forward 3 | TM | N in |
| 83 | LG:232190.1:2000SEP08 | 936 | 1022 | forward 3 | TM | N in |
| 84 | LG:1068127.1:2000SEP08 | 423 | 470 | forward 3 | TM | |
| 85 | LG:408751.3:2000SEP08 | 2194 | 2253 | forward 1 | TM | N out |
| 85 | LG:408751.3:2000SEP08 | 2320 | 2406 | forward 1 | TM | N out |
| 85 | LG:408751.3:2000SEP08 | 1907 | 1993 | forward 2 | TM | N out |
| 85 | LG:408751.3:2000SEP08 | 2378 | 2464 | forward 2 | TM | N out |
| 85 | LG:408751.3:2000SEP08 | 1866 | 1928 | forward 3 | TM | N in |
| 85 | LG:408751.3:2000SEP08 | 2328 | 2414 | forward 3 | TM | N in |
| 86 | LG:1078933.1:2000SEP08 | 905 | 991 | forward 2 | TM | N in |
| 86 | LG:1078933.1:2000SEP08 | 924 | 1010 | forward 3 | TM | N in |
| 87 | LG:958731.1:2000SEP08 | 15 | 101 | forward 3 | TM | N out |
| 88 | LG:024125.5:2000SEP08 | 474 | 554 | forward 3 | TM | |
| 89 | LG:373637.3:2000SEP08 | 473 | 559 | forward 2 | TM | N in |
| 89 | LG:373637.3:2000SEP08 | 854 | 934 | forward 2 | TM | N in |
| 90 | LG:1053229.1:2000SEP08 | 410 | 487 | forward 2 | TM | N out |
| 91 | LG:248364.1:2000SEP08 | 439 | 495 | forward 1 | TM | N out |
| 92 | LG:477130.1:2000SEP08 | 37 | 108 | forward 1 | TM | N in |
| 92 | LG:477130.1:2000SEP08 | 406 | 489 | forward 1 | TM | N in |
| 92 | LG:477130.1:2000SEP08 | 685 | 759 | forward 1 | TM | N in |
| 92 | LG:477130.1:2000SEP08 | 641 | 727 | forward 2 | TM | N in |
| 92 | LG:477130.1:2000SEP08 | 741 | 827 | forward 3 | TM | N in |
| 93 | LG:113786.17:2000SEP08 | 89 | 139 | forward 2 | TM | N out |
| 93 | LG:113786.17:2000SEP08 | 18 | 68 | forward 3 | TM | |
| 94 | LG:347635.1:2000SEP08 | 220 | 294 | forward 1 | TM | N out |
| 94 | LG:347635.1:2000SEP08 | 406 | 456 | forward 1 | TM | N out |
| 94 | LG:347635.1:2000SEP08 | 479 | 550 | forward 2 | TM | N out |
| 94 | LG:347635.1:2000SEP08 | 1283 | 1369 | forward 2 | TM | N out |
| 94 | LG:347635.1:2000SEP08 | 615 | 701 | forward 3 | TM | N out |
| 94 | LG:347635.1:2000SEP08 | 1347 | 1397 | forward 3 | TM | N out |
| 95 | LG:242966.4:2000SEP08 | 1439 | 1510 | forward 2 | TM | N in |
| 96 | LG:217814.1:2000SEP08 | 1621 | 1689 | forward 1 | TM | N out |
| 96 | LG:217814.1:2000SEP08 | 1622 | 1708 | forward 2 | TM | N out |
| 96 | LG:217814.1:2000SEP08 | 246 | 302 | forward 3 | TM | N in |
| 96 | LG:217814.1:2000SEP08 | 1695 | 1772 | forward 3 | TM | N in |
| 97 | LG:476452.1:2000SEP08 | 556 | 624 | forward 1 | TM | N out |
| 97 | LG:476452.1:2000SEP08 | 988 | 1074 | forward 1 | TM | N out |
| 98 | LG:1100657.1:2000SEP08 | 31 | 117 | forward 1 | TM | N out |
| 99 | LG:1132418.2:2000SEP08 | 43 | 129 | forward 1 | TM | N out |
| 99 | LG:1132418.2:2000SEP08 | 244 | 327 | forward 1 | TM | N out |
| 100 | LG:1098570.1:2000SEP08 | 400 | 453 | forward 1 | TM | N out |
| 100 | LG:1098570.1:2000SEP08 | 26 | 85 | forward 2 | TM | N out |
| 101 | LG:1097987.1:2000SEP08 | 152 | 208 | forward 2 | TM | N out |
| 101 | LG:1097987.1:2000SEP08 | 141 | 215 | forward 3 | TM | |
| 101 | LG:1097987.1:2000SEP08 | 912 | 971 | forward 3 | TM | |
| 102 | LG:337818.2:2000SEP08 | 40 | 117 | forward 1 | TM | N out |
| 102 | LG:337818.2:2000SEP08 | 532 | 618 | forward 1 | TM | N out |
| 102 | LG:337818.2:2000SEP08 | 907 | 993 | forward 1 | TM | N out |
| 102 | LG:337818.2:2000SEP08 | 1360 | 1425 | forward 1 | TM | N out |
| 103 | LG:1040582.1:2000SEP08 | 31 | 117 | forward 1 | TM | N in |
| 103 | LG:1040582.1:2000SEP08 | 539 | 595 | forward 2 | TM | N out |
| 103 | LG:1040582.1:2000SEP08 | 330 | 416 | forward 3 | TM | N out |
| 104 | LG:1099122.1:2000SEP08 | 385 | 456 | forward 1 | TM | N out |
| 104 | LG:1099122.1:2000SEP08 | 236 | 292 | forward 2 | TM | N in |
| 104 | LG:1099122.1:2000SEP08 | 350 | 436 | forward 2 | TM | N in |
| 104 | LG:1099122.1:2000SEP08 | 258 | 308 | forward 3 | TM | N out |
| 104 | LG:1099122.1:2000SEP08 | 420 | 470 | forward 3 | TM | N out |
| 105 | LG:1327449.1:2000SEP08 | 112 | 198 | forward 1 | TM | N out |
| 105 | LG:1327449.1:2000SEP08 | 268 | 318 | forward 1 | TM | N out |
| 105 | LG:1327449.1:2000SEP08 | 395 | 481 | forward 2 | TM | N out |
| 106 | LG:227933.5:2000SEP08 | 1114 | 1200 | forward 1 | TM | N in |
| 106 | LG:227933.5:2000SEP08 | 1231 | 1290 | forward 1 | TM | N in |
| 106 | LG:227933.5:2000SEP08 | 255 | 308 | forward 3 | TM | N out |
| 107 | LG:1043709.2:2000SEP08 | 600 | 650 | forward 3 | TM | N out |
| 108 | LG:1099871.1:2000SEP08 | 181 | 243 | forward 1 | TM | N out |
| 109 | LG:1399139.4:2000SEP08 | 289 | 369 | forward 1 | TM | N out |
| 109 | LG:1399139.4:2000SEP08 | 616 | 684 | forward 1 | TM | N out |
| 109 | LG:1399139.4:2000SEP08 | 185 | 235 | forward 2 | TM | |
| 109 | LG:1399139.4:2000SEP08 | 555 | 641 | forward 3 | TM | N out |
| 110 | LG:236386.1:2000SEP08 | 106 | 171 | forward 1 | TM | N out |
| 110 | LG:236386.1:2000SEP08 | 271 | 357 | forward 1 | TM | N out |
| 110 | LG:236386.1:2000SEP08 | 3097 | 3159 | forward 1 | TM | N out |
| 110 | LG:236386.1:2000SEP08 | 3715 | 3789 | forward 1 | TM | N out |
| 110 | LG:236386.1:2000SEP08 | 3093 | 3164 | forward 3 | TM | N out |
| 110 | LG:236386.1:2000SEP08 | 3759 | 3812 | forward 3 | TM | N out |
| 111 | LG:1015157.1:2000SEP08 | 320 | 406 | forward 2 | TM | N in |
| 112 | LG:1065433.1:2000SEP08 | 790 | 876 | forward 1 | TM | N in |
| 113 | LG:236992.4:2000SEP08 | 421 | 471 | forward 1 | TM | N out |
| 113 | LG:236992.4:2000SEP08 | 14 | 100 | forward 2 | TM | N out |
| 114 | LG:1071124.1:2000SEP08 | 171 | 257 | forward 3 | TM | N out |
| 115 | LG:206425.2:2000SEP08 | 175 | 237 | forward 1 | TM | N in |
| 115 | LG:206425.2:2000SEP08 | 424 | 483 | forward 1 | TM | N in |
| 115 | LG:206425.2:2000SEP08 | 1810 | 1896 | forward 1 | TM | N in |
| 115 | LG:206425.2:2000SEP08 | 1939 | 2001 | forward 1 | TM | N in |
| 115 | LG:206425.2:2000SEP08 | 404 | 457 | forward 2 | TM | N in |
| 115 | LG:206425.2:2000SEP08 | 2168 | 2254 | forward 2 | TM | N in |
| 115 | LG:206425.2:2000SEP08 | 561 | 620 | forward 3 | TM | N out |
| 115 | LG:206425.2:2000SEP08 | 1908 | 1994 | forward 3 | TM | N out |
| 116 | LG:885747.2:2000SEP08 | 125 | 205 | forward 2 | TM | N in |
| 116 | LG:885747.2:2000SEP08 | 135 | 191 | forward 3 | TM | N out |
| 116 | LG:885747.2:2000SEP08 | 189 | 239 | forward 3 | TM | N out |
| 117 | LG:1140501.1:2000SEP08 | 622 | 708 | forward 1 | TM | N out |
| 117 | LG:1140501.1:2000SEP08 | 844 | 918 | forward 1 | TM | N out |
| 117 | LG:1140501.1:2000SEP08 | 1138 | 1206 | forward 1 | TM | N out |
| 117 | LG:1140501.1:2000SEP08 | 86 | 169 | forward 2 | TM | N out |
| 117 | LG:1140501.1:2000SEP08 | 221 | 301 | forward 2 | TM | N out |
| 117 | LG:1140501.1:2000SEP08 | 617 | 703 | forward 2 | TM | N out |
| 117 | LG:1140501.1:2000SEP08 | 606 | 677 | forward 3 | TM | N in |
| 118 | LG:001239.1:2000SEP08 | 1048 | 1122 | forward 1 | TM | N out |
| 118 | LG:001239.1:2000SEP08 | 669 | 755 | forward 3 | TM | N out |
| 118 | LG:001239.1:2000SEP08 | 1911 | 1976 | forward 3 | TM | N out |
| 119 | LG:018980.1:2000SEP08 | 124 | 204 | forward 1 | TM | N out |
| 119 | LG:018980.1:2000SEP08 | 944 | 997 | forward 2 | TM | N out |
| 119 | LG:018980.1:2000SEP08 | 405 | 464 | forward 3 | TM | N out |
| 119 | LG:018980.1:2000SEP08 | 900 | 986 | forward 3 | TM | N out |
| 119 | LG:018980.1:2000SEP08 | 1017 | 1103 | forward 3 | TM | N out |
| 120 | LG:1083120.3:2000SEP08 | 214 | 291 | forward 1 | TM | N out |
| 120 | LG:1083120.3:2000SEP08 | 233 | 319 | forward 2 | TM | N out |
| 120 | LG:1083120.3:2000SEP08 | 252 | 320 | forward 3 | TM | N in |
| 121 | LG:233258.3:2000SEP08 | 58 | 141 | forward 1 | TM | |
| 121 | LG:233258.3:2000SEP08 | 1783 | 1842 | forward 1 | TM | |
| 121 | LG:233258.3:2000SEP08 | 2248 | 2322 | forward 1 | TM | |
| 121 | LG:233258.3:2000SEP08 | 4522 | 4596 | forward 1 | TM | |
| 121 | LG:233258.3:2000SEP08 | 4208 | 4294 | forward 2 | TM | N out |
| 121 | LG:233258.3:2000SEP08 | 4478 | 4534 | forward 2 | TM | N out |
| 121 | LG:233258.3:2000SEP08 | 390 | 476 | forward 3 | TM | N in |
| 121 | LG:233258.3:2000SEP08 | 2766 | 2852 | forward 3 | TM | N in |
| 122 | LG:999062.1:2000SEP08 | 455 | 508 | forward 2 | TM | N in |
| 122 | LG:999062.1:2000SEP08 | 510 | 596 | forward 3 | TM | N in |
| 123 | LG:887776.1:2000SEP08 | 14 | 91 | forward 2 | TM | N out |
| 124 | LG:1400301.2:2000SEP08 | 445 | 531 | forward 1 | TM | N out |
| 124 | LG:1400301.2:2000SEP08 | 456 | 518 | forward 3 | TM | N out |
| 125 | LG:1329362.1:2000SEP08 | 18 | 104 | forward 3 | TM | N out |
| 126 | LG:1096498.1:2000SEP08 | 137 | 199 | forward 2 | TM | N out |
| 126 | LG:1096498.1:2000SEP08 | 201 | 269 | forward 3 | TM | N out |
| 126 | LG:1096498.1:2000SEP08 | 321 | 371 | forward 3 | TM | N out |
| 127 | LG:1096337.1:2000SEP08 | 625 | 711 | forward 1 | TM | N out |
| 127 | LG:1096337.1:2000SEP08 | 500 | 553 | forward 2 | TM | |
| 128 | LG:1400579.1:2000SEP08 | 797 | 883 | forward 2 | TM | N out |
| 128 | LG:1400579.1:2000SEP08 | 9 | 86 | forward 3 | TM | N out |
| 128 | LG:1400579.1:2000SEP08 | 669 | 755 | forward 3 | TM | N out |
| 129 | LG:1080091.1:2000SEP08 | 67 | 114 | forward 1 | TM | N out |
| 129 | LG:1080091.1:2000SEP08 | 435 | 497 | forward 3 | TM | N out |
| 130 | LG:1082203.1:2000SEP08 | 1438 | 1521 | forward 1 | TM | N in |
| 130 | LG:1082203.1:2000SEP08 | 155 | 217 | forward 2 | TM | N out |
| 130 | LG:1082203.1:2000SEP08 | 272 | 358 | forward 2 | TM | N out |
| 131 | LG:1084051.1:2000SEP08 | 301 | 366 | forward 1 | TM | N out |
| 131 | LG:1084051.1:2000SEP08 | 934 | 1017 | forward 1 | TM | N out |
| 131 | LG:1084051.1:2000SEP08 | 1072 | 1140 | forward 1 | TM | N out |
| 131 | LG:1084051.1:2000SEP08 | 875 | 961 | forward 2 | TM | N out |
| 131 | LG:1084051.1:2000SEP08 | 882 | 968 | forward 3 | TM | N in |
| 131 | LG:1084051.1:2000SEP08 | 1071 | 1151 | forward 3 | TM | N in |
| 132 | LG:1082393.1:2000SEP08 | 505 | 579 | forward 1 | TM | N in |
| 132 | LG:1082393.1:2000SEP08 | 1710 | 1784 | forward 3 | TM | N in |
| 133 | LG:1086183.1:2000SEP08 | 1093 | 1179 | forward 1 | TM | N out |
| 133 | LG:1086183.1:2000SEP08 | 1166 | 1252 | forward 2 | TM | N in |
| 133 | LG:1086183.1:2000SEP08 | 15 | 86 | forward 3 | TM | |
| 133 | LG:1086183.1:2000SEP08 | 381 | 437 | forward 3 | TM | |
| 133 | LG:1086183.1:2000SEP08 | 1194 | 1280 | forward 3 | TM | |
| 134 | LG:1090268.1:2000SEP08 | 1882 | 1953 | forward 1 | TM | N out |
| 134 | LG:1090268.1:2000SEP08 | 1969 | 2055 | forward 1 | TM | N out |
| 134 | LG:1090268.1:2000SEP08 | 1670 | 1729 | forward 2 | TM | N out |
| 134 | LG:1090268.1:2000SEP08 | 1853 | 1939 | forward 2 | TM | N out |
| 134 | LG:1090268.1:2000SEP08 | 1997 | 2083 | forward 2 | TM | N out |
| 134 | LG:1090268.1:2000SEP08 | 1485 | 1550 | forward 3 | TM | N in |
| 134 | LG:1090268.1:2000SEP08 | 1869 | 1943 | forward 3 | TM | N in |
| 134 | LG:1090268.1:2000SEP08 | 1962 | 2048 | forward 3 | TM | N in |
| 135 | LG:1400597.5:2000SEP08 | 134 | 199 | forward 2 | TM | N in |
| 136 | LG:1080307.2:2000SEP08 | 275 | 346 | forward 2 | TM | N out |
| 136 | LG:1080307.2:2000SEP08 | 347 | 403 | forward 2 | TM | N out |
| 136 | LG:1080307.2:2000SEP08 | 183 | 269 | forward 3 | TM | N in |
| 136 | LG:1080307.2:2000SEP08 | 303 | 371 | forward 3 | TM | N in |
| 137 | LG:1400603.2:2000SEP08 | 792 | 878 | forward 3 | TM | N in |
| 137 | LG:1400603.2:2000SEP08 | 903 | 971 | forward 3 | TM | N in |
| 137 | LG:1400603.2:2000SEP08 | 1068 | 1151 | forward 3 | TM | N in |
| 138 | LG:1052984.1:2000SEP08 | 496 | 582 | forward 1 | TM | N in |
| 138 | LG:1052984.1:2000SEP08 | 509 | 595 | forward 2 | TM | N out |
| 138 | LG:1052984.1:2000SEP08 | 495 | 581 | forward 3 | TM | |
| 139 | LG:1091259.1:2000SEP08 | 799 | 885 | forward 1 | TM | N in |
| 140 | LG:1082263.2:2000SEP08 | 1390 | 1458 | forward 1 | TM | N in |
| 140 | LG:1082263.2:2000SEP08 | 1558 | 1644 | forward 1 | TM | N in |
| 140 | LG:1082263.2:2000SEP08 | 83 | 169 | forward 2 | TM | |
| 140 | LG:1082263.2:2000SEP08 | 1526 | 1612 | forward 2 | TM | |
| 140 | LG:1082263.2:2000SEP08 | 1578 | 1664 | forward 3 | TM | N in |
| 141 | LG:1048604.2:2000SEP08 | 562 | 618 | forward 1 | TM | N in |
| 141 | LG:1048604.2:2000SEP08 | 697 | 768 | forward 1 | TM | N in |
| 141 | LG:1048604.2:2000SEP08 | 856 | 930 | forward 1 | TM | N in |
| 141 | LG:1048604.2:2000SEP08 | 332 | 418 | forward 2 | TM | N in |
| 141 | LG:1048604.2:2000SEP08 | 689 | 775 | forward 2 | TM | N in |
| 141 | LG:1048604.2:2000SEP08 | 1115 | 1192 | forward 2 | TM | N in |
| 141 | LG:1048604.2:2000SEP08 | 483 | 557 | forward 3 | TM | N in |
| 141 | LG:1048604.2:2000SEP08 | 570 | 638 | forward 3 | TM | N in |
| 141 | LG:1048604.2:2000SEP08 | 678 | 743 | forward 3 | TM | N in |
| 141 | LG:1048604.2:2000SEP08 | 1113 | 1199 | forward 3 | TM | N in |
| 142 | LG:1085254.3:2000SEP08 | 331 | 378 | forward 1 | TM | N in |
| 142 | LG:1085254.3:2000SEP08 | 204 | 290 | forward 3 | TM | N out |
| 143 | LG:1400606.2:2000SEP08 | 1096 | 1176 | forward 1 | TM | N out |
| 143 | LG:1400606.2:2000SEP08 | 794 | 880 | forward 2 | TM | N in |
| 143 | LG:1400606.2:2000SEP08 | 1031 | 1117 | forward 2 | TM | N in |
| 143 | LG:1400606.2:2000SEP08 | 93 | 173 | forward 3 | TM | N in |
| 143 | LG:1400606.2:2000SEP08 | 765 | 851 | forward 3 | TM | N in |
| 144 | LG:1090358.2:2000SEP08 | 250 | 336 | forward 1 | TM | N in |
| 144 | LG:1090358.2:2000SEP08 | 758 | 844 | forward 2 | TM | N out |
| 144 | LG:1090358.2:2000SEP08 | 848 | 919 | forward 2 | TM | N out |
| 144 | LG:1090358.2:2000SEP08 | 974 | 1060 | forward 2 | TM | N out |
| 145 | LG:1079064.2:2000SEP08 | 862 | 936 | forward 1 | TM | N out |
| 146 | LG:1076866.1:2000SEP08 | 2113 | 2187 | forward 1 | TM | N in |
| 146 | LG:1076866.1:2000SEP08 | 2111 | 2170 | forward 2 | TM | |
| 146 | LG:1076866.1:2000SEP08 | 501 | 587 | forward 3 | TM | N in |
| 146 | LG:1076866.1:2000SEP08 | 2148 | 2234 | forward 3 | TM | N in |
| 147 | LG:969359.1:2000SEP08 | 256 | 303 | forward 1 | TM | N out |
| 147 | LG:969359.1:2000SEP08 | 276 | 350 | forward 3 | TM | N out |
| 148 | LG:366783.1:2000SEP08 | 1060 | 1143 | forward 1 | TM | N in |
| 148 | LG:366783.1:2000SEP08 | 470 | 550 | forward 2 | TM | |
| 148 | LG:366783.1:2000SEP08 | 1050 | 1109 | forward 3 | TM | N in |
| 149 | LG:332176.3:2000SEP08 | 442 | 495 | forward 1 | TM | N in |
| 149 | LG:332176.3:2000SEP08 | 209 | 295 | forward 2 | TM | N in |
| 149 | LG:332176.3:2000SEP08 | 234 | 299 | forward 3 | TM | N in |
| 149 | LG:332176.3:2000SEP08 | 792 | 851 | forward 3 | TM | N in |
| 149 | LG:332176.3:2000SEP08 | 876 | 938 | forward 3 | TM | N in |
| 149 | LG:332176.3:2000SEP08 | 966 | 1028 | forward 3 | TM | N in |
| 150 | LG:994938.1:2000SEP08 | 562 | 618 | forward 1 | TM | N out |
| 150 | LG:994938.1:2000SEP08 | 287 | 343 | forward 2 | TM | N out |
| 150 | LG:994938.1:2000SEP08 | 512 | 598 | forward 2 | TM | N out |
| 150 | LG:994938.1:2000SEP08 | 279 | 356 | forward 3 | TM | N out |
| 150 | LG:994938.1:2000SEP08 | 474 | 557 | forward 3 | TM | N out |
| 151 | LG:982800.1:2000SEP08 | 25 | 81 | forward 1 | TM | N out |
| 151 | LG:982800.1:2000SEP08 | 1708 | 1782 | forward 1 | TM | N out |
| 151 | LG:982800.1:2000SEP08 | 2305 | 2391 | forward 1 | TM | N out |
| 151 | LG:982800.1:2000SEP08 | 1658 | 1744 | forward 2 | TM | N in |
| 151 | LG:982800.1:2000SEP08 | 1880 | 1927 | forward 2 | TM | N in |
| 151 | LG:982800.1:2000SEP08 | 2255 | 2341 | forward 2 | TM | N in |
| 151 | LG:982800.1:2000SEP08 | 1614 | 1697 | forward 3 | TM | N in |
| 151 | LG:982800.1:2000SEP08 | 2220 | 2306 | forward 3 | TM | N in |
| 152 | LG:977850.7:2000SEP08 | 58 | 144 | forward 1 | TM | N out |
| 152 | LG:977850.7:2000SEP08 | 187 | 237 | forward 1 | TM | N out |
| 152 | LG:977850.7:2000SEP08 | 35 | 112 | forward 2 | TM | N out |
| 152 | LG:977850.7:2000SEP08 | 48 | 116 | forward 3 | TM | N in |
| 153 | LG:234748.2:2000SEP08 | 25 | 99 | forward 1 | TM | N out |
| 153 | LG:234748.2:2000SEP08 | 601 | 663 | forward 1 | TM | N out |
| 153 | LG:234748.2:2000SEP08 | 679 | 741 | forward 1 | TM | N out |
| 153 | LG:234748.2:2000SEP08 | 871 | 957 | forward 1 | TM | N out |
| 153 | LG:234748.2:2000SEP08 | 1162 | 1230 | forward 1 | TM | N out |
| 153 | LG:234748.2:2000SEP08 | 1237 | 1302 | forward 1 | TM | N out |
| 153 | LG:234748.2:2000SEP08 | 1579 | 1641 | forward 1 | TM | N out |
| 153 | LG:234748.2:2000SEP08 | 1984 | 2070 | forward 1 | TM | N out |
| 153 | LG:234748.2:2000SEP08 | 2110 | 2196 | forward 1 | TM | N out |
| 153 | LG:234748.2:2000SEP08 | 2308 | 2355 | forward 1 | TM | N out |
| 153 | LG:234748.2:2000SEP08 | 359 | 421 | forward 2 | TM | |
| 153 | LG:234748.2:2000SEP08 | 464 | 526 | forward 2 | TM | |
| 153 | LG:234748.2:2000SEP08 | 656 | 742 | forward 2 | TM | |
| 153 | LG:234748.2:2000SEP08 | 899 | 949 | forward 2 | TM | |
| 153 | LG:234748.2:2000SEP08 | 995 | 1057 | forward 2 | TM | |
| 153 | LG:234748.2:2000SEP08 | 1076 | 1138 | forward 2 | TM | |
| 153 | LG:234748.2:2000SEP08 | 1412 | 1489 | forward 2 | TM | |
| 153 | LG:234748.2:2000SEP08 | 1925 | 1999 | forward 2 | TM | |
| 153 | LG:234748.2:2000SEP08 | 2012 | 2071 | forward 2 | TM | |
| 153 | LG:234748.2:2000SEP08 | 2105 | 2191 | forward 2 | TM | |
| 153 | LG:234748.2:2000SEP08 | 2204 | 2290 | forward 2 | TM | |
| 153 | LG:234748.2:2000SEP08 | 393 | 455 | forward 3 | TM | N in |
| 153 | LG:234748.2:2000SEP08 | 474 | 536 | forward 3 | TM | N in |
| 153 | LG:234748.2:2000SEP08 | 645 | 725 | forward 3 | TM | N in |
| 153 | LG:234748.2:2000SEP08 | 801 | 863 | forward 3 | TM | N in |
| 153 | LG:234748.2:2000SEP08 | 894 | 956 | forward 3 | TM | N in |
| 153 | LG:234748.2:2000SEP08 | 1485 | 1535 | forward 3 | TM | N in |
| 153 | LG:234748.2:2000SEP08 | 2052 | 2114 | forward 3 | TM | N in |
| 153 | LG:234748.2:2000SEP08 | 2145 | 2207 | forward 3 | TM | N in |
| 153 | LG:234748.2:2000SEP08 | 2238 | 2300 | forward 3 | TM | N in |
| 154 | LG:306284.1:2000SEP08 | 255 | 305 | forward 3 | TM | N in |
| 177 | LI:335923.2:2000SEP08 | 149 | 229 | forward 2 | TM | N in |
| 178 | LI:345884.1:2000SEP08 | 61 | 147 | forward 1 | TM | N out |
| 179 | LI:417127.1:2000SEP08 | 148 | 201 | forward 1 | TM | N in |
| 179 | LI:417127.1:2000SEP08 | 114 | 200 | forward 3 | TM | N in |
| 180 | LI:451710.1:2000SEP08 | 502 | 588 | forward 1 | TM | N in |
| 180 | LI:451710.1:2000SEP08 | 453 | 515 | forward 3 | TM | N in |
| 181 | LI:406882.2:2000SEP08 | 109 | 180 | forward 1 | TM | N in |
| 181 | LI:406882.2:2000SEP08 | 514 | 597 | forward 1 | TM | N in |
| 181 | LI:406882.2:2000SEP08 | 1045 | 1101 | forward 1 | TM | N in |
| 181 | LI:406882.2:2000SEP08 | 293 | 379 | forward 2 | TM | N out |
| 181 | LI:406882.2:2000SEP08 | 653 | 739 | forward 2 | TM | N out |
| 181 | LI:406882.2:2000SEP08 | 1091 | 1141 | forward 2 | TM | N out |
| 181 | LI:406882.2:2000SEP08 | 675 | 746 | forward 3 | TM | N out |
| 182 | LI:728223.1:2000SEP08 | 45 | 131 | forward 3 | TM | N out |
| 182 | LI:728223.1:2000SEP08 | 129 | 179 | forward 3 | TM | N out |
| 183 | LI:289783.19:2000SEP08 | 10 | 93 | forward 1 | TM | N in |
| 183 | LI:289783.19:2000SEP08 | 151 | 204 | forward 1 | TM | N in |
| 183 | LI:289783.19:2000SEP08 | 11 | 94 | forward 2 | TM | |
| 183 | LI:289783.19:2000SEP08 | 140 | 226 | forward 2 | TM | |
| 183 | LI:289783.19:2000SEP08 | 489 | 575 | forward 3 | TM | N in |
| 184 | LI:235255.8:2000SEP08 | 985 | 1044 | forward 1 | TM | N out |
| 185 | LI:237693.5:2000SEP08 | 355 | 429 | forward 1 | TM | N in |
| 186 | LI:433670.3:2000SEP08 | 79 | 165 | forward 1 | TM | |
| 186 | LI:433670.3:2000SEP08 | 598 | 681 | forward 1 | TM | |
| 186 | LI:433670.3:2000SEP08 | 796 | 867 | forward 1 | TM | |
| 186 | LI:433670.3:2000SEP08 | 74 | 160 | forward 2 | TM | N in |
| 186 | LI:433670.3:2000SEP08 | 221 | 292 | forward 2 | TM | N in |
| 186 | LI:433670.3:2000SEP08 | 662 | 736 | forward 2 | TM | N in |
| 186 | LI:433670.3:2000SEP08 | 848 | 898 | forward 2 | TM | N in |
| 186 | LI:433670.3:2000SEP08 | 75 | 161 | forward 3 | TM | N in |
| 186 | LI:433670.3:2000SEP08 | 387 | 449 | forward 3 | TM | N in |
| 186 | LI:433670.3:2000SEP08 | 651 | 737 | forward 3 | TM | N in |
| 187 | LI:202943.4:2000SEP08 | 535 | 609 | forward 1 | TM | N out |
| 187 | LI:202943.4:2000SEP08 | 1570 | 1656 | forward 1 | TM | N out |
| 187 | LI:202943.4:2000SEP08 | 342 | 395 | forward 3 | TM | N out |
| 187 | LI:202943.4:2000SEP08 | 495 | 557 | forward 3 | TM | N out |
| 187 | LI:202943.4:2000SEP08 | 579 | 641 | forward 3 | TM | N out |
| 187 | LI:202943.4:2000SEP08 | 759 | 845 | forward 3 | TM | N out |
| 187 | LI:202943.4:2000SEP08 | 1653 | 1721 | forward 3 | TM | N out |
| 187 | LI:202943.4:2000SEP08 | 1905 | 1991 | forward 3 | TM | N out |
| 188 | LI:068682.1:2000SEP08 | 700 | 786 | forward 1 | TM | N out |
| 189 | LI:203301.3:2000SEP08 | 643 | 729 | forward 1 | TM | N out |
| 189 | LI:203301.3:2000SEP08 | 1510 | 1572 | forward 1 | TM | N out |
| 189 | LI:203301.3:2000SEP08 | 1585 | 1647 | forward 1 | TM | N out |
| 189 | LI:203301.3:2000SEP08 | 1774 | 1842 | forward 1 | TM | N out |
| 189 | LI:203301.3:2000SEP08 | 2014 | 2100 | forward 1 | TM | N out |
| 189 | LI:203301.3:2000SEP08 | 2158 | 2220 | forward 1 | TM | N out |
| 189 | LI:203301.3:2000SEP08 | 2383 | 2469 | forward 1 | TM | N out |
| 189 | LI:203301.3:2000SEP08 | 2548 | 2634 | forward 1 | TM | N out |
| 189 | LI:203301.3:2000SEP08 | 2662 | 2724 | forward 1 | TM | N out |
| 189 | LI:203301.3:2000SEP08 | 2749 | 2835 | forward 1 | TM | N out |
| 189 | LI:203301.3:2000SEP08 | 1685 | 1771 | forward 2 | TM | N in |
| 189 | LI:203301.3:2000SEP08 | 2162 | 2233 | forward 2 | TM | N in |
| 189 | LI:203301.3:2000SEP08 | 2342 | 2428 | forward 2 | TM | N in |
| 189 | LI:203301.3:2000SEP08 | 2519 | 2572 | forward 2 | TM | N in |
| 189 | LI:203301.3:2000SEP08 | 2597 | 2674 | forward 2 | TM | N in |
| 189 | LI:203301.3:2000SEP08 | 117 | 170 | forward 3 | TM | N in |
| 189 | LI:203301.3:2000SEP08 | 387 | 470 | forward 3 | TM | N in |
| 189 | LI:203301.3:2000SEP08 | 1086 | 1136 | forward 3 | TM | N in |
| 189 | LI:203301.3:2000SEP08 | 1344 | 1430 | forward 3 | TM | N in |
| 189 | LI:203301.3:2000SEP08 | 1626 | 1712 | forward 3 | TM | N in |
| 189 | LI:203301.3:2000SEP08 | 2364 | 2435 | forward 3 | TM | N in |
| 189 | LI:203301.3:2000SEP08 | 2604 | 2672 | forward 3 | TM | N in |
| 190 | LI:020726.3:2000SEP08 | 1363 | 1449 | forward 1 | TM | N in |
| 190 | LI:020726.3:2000SEP08 | 1795 | 1863 | forward 1 | TM | N in |
| 190 | LI:020726.3:2000SEP08 | 1930 | 1992 | forward 1 | TM | N in |
| 190 | LI:020726.3:2000SEP08 | 545 | 631 | forward 2 | TM | N in |
| 190 | LI:020726.3:2000SEP08 | 656 | 742 | forward 2 | TM | N in |
| 190 | LI:020726.3:2000SEP08 | 767 | 829 | forward 2 | TM | N in |
| 190 | LI:020726.3:2000SEP08 | 839 | 901 | forward 2 | TM | N in |
| 190 | LI:020726.3:2000SEP08 | 938 | 1024 | forward 2 | TM | N in |
| 190 | LI:020726.3:2000SEP08 | 1391 | 1477 | forward 2 | TM | N in |
| 190 | LI:020726.3:2000SEP08 | 1598 | 1672 | forward 2 | TM | N in |
| 190 | LI:020726.3:2000SEP08 | 1790 | 1852 | forward 2 | TM | N in |
| 190 | LI:020726.3:2000SEP08 | 1874 | 1936 | forward 2 | TM | N in |
| 190 | LI:020726.3:2000SEP08 | 1958 | 2020 | forward 2 | TM | N in |
| 191 | LI:027209.1:2000SEP08 | 25 | 111 | forward 1 | TM | N out |
| 191 | LI:027209.1:2000SEP08 | 1129 | 1203 | forward 1 | TM | N out |
| 191 | LI:027209.1:2000SEP08 | 1219 | 1305 | forward 1 | TM | N out |
| 191 | LI:027209.1:2000SEP08 | 1447 | 1533 | forward 1 | TM | N out |
| 191 | LI:027209.1:2000SEP08 | 413 | 475 | forward 2 | TM | N out |
| 191 | LI:027209.1:2000SEP08 | 527 | 601 | forward 2 | TM | N out |
| 191 | LI:027209.1:2000SEP08 | 659 | 739 | forward 2 | TM | N out |
| 191 | LI:027209.1:2000SEP08 | 785 | 847 | forward 2 | TM | N out |
| 191 | LI:027209.1:2000SEP08 | 1178 | 1234 | forward 2 | TM | N out |
| 191 | LI:027209.1:2000SEP08 | 1232 | 1309 | forward 2 | TM | N out |
| 191 | LI:027209.1:2000SEP08 | 1379 | 1465 | forward 2 | TM | N out |
| 191 | LI:027209.1:2000SEP08 | 510 | 563 | forward 3 | TM | N in |
| 191 | LI:027209.1:2000SEP08 | 882 | 938 | forward 3 | TM | N in |
| 191 | LI:027209.1:2000SEP08 | 945 | 1016 | forward 3 | TM | N in |
| 191 | LI:027209.1:2000SEP08 | 1041 | 1112 | forward 3 | TM | N in |
| 191 | LI:027209.1:2000SEP08 | 1146 | 1208 | forward 3 | TM | N in |
| 191 | LI:027209.1:2000SEP08 | 1224 | 1286 | forward 3 | TM | N in |
| 192 | LI:108819.1:2000SEP08 | 196 | 264 | forward 1 | TM | N in |
| 192 | LI:108819.1:2000SEP08 | 203 | 271 | forward 2 | TM | |
| 192 | LI:108819.1:2000SEP08 | 290 | 343 | forward 2 | TM | |
| 193 | LI:021759.1:2000SEP08 | 133 | 186 | forward 1 | TM | N out |
| 193 | LI:021759.1:2000SEP08 | 59 | 115 | forward 2 | TM | N out |
| 193 | LI:021759.1:2000SEP08 | 495 | 581 | forward 3 | TM | N in |
| 193 | LI:021759.1:2000SEP08 | 981 | 1067 | forward 3 | TM | N in |
| 194 | LI:1165967.1:2000SEP08 | 323 | 403 | forward 2 | TM | N out |
| 195 | LI:1166315.1:2000SEP08 | 693 | 749 | forward 3 | TM | N out |
| 196 | LI:204626.1:2000SEP08 | 19 | 99 | forward 1 | TM | N out |
| 197 | LI:801140.1:2000SEP08 | 421 | 498 | forward 1 | TM | N in |
| 198 | LI:286639.1:2000SEP08 | 127 | 213 | forward 1 | TM | N out |
| 198 | LI:286639.1:2000SEP08 | 982 | 1068 | forward 1 | TM | N out |
| 198 | LI:286639.1:2000SEP08 | 1879 | 1944 | forward 1 | TM | N out |
| 198 | LI:286639.1:2000SEP08 | 62 | 148 | forward 2 | TM | N out |
| 198 | LI:286639.1:2000SEP08 | 965 | 1027 | forward 2 | TM | N out |
| 198 | LI:286639.1:2000SEP08 | 1061 | 1123 | forward 2 | TM | N out |
| 198 | LI:286639.1:2000SEP08 | 1589 | 1663 | forward 2 | TM | N out |
| 198 | LI:286639.1:2000SEP08 | 1787 | 1873 | forward 2 | TM | N out |
| 198 | LI:286639.1:2000SEP08 | 267 | 320 | forward 3 | TM | N out |
| 198 | LI:286639.1:2000SEP08 | 1794 | 1847 | forward 3 | TM | N out |
| 199 | LI:288905.4:2000SEP08 | 868 | 927 | forward 1 | TM | N out |
| 199 | LI:288905.4:2000SEP08 | 1552 | 1638 | forward 1 | TM | N out |
| 199 | LI:288905.4:2000SEP08 | 1913 | 1975 | forward 2 | TM | N out |
| 199 | LI:288905.4:2000SEP08 | 2000 | 2062 | forward 2 | TM | N out |
| 199 | LI:288905.4:2000SEP08 | 99 | 158 | forward 3 | TM | N in |
| 200 | LI:332161.1:2000SEP08 | 1507 | 1587 | forward 1 | TM | N in |
| 200 | LI:332161.1:2000SEP08 | 1663 | 1743 | forward 1 | TM | N in |
| 200 | LI:332161.1:2000SEP08 | 2314 | 2400 | forward 1 | TM | N in |
| 200 | LI:332161.1:2000SEP08 | 2890 | 2940 | forward 1 | TM | N in |
| 200 | LI:332161.1:2000SEP08 | 776 | 835 | forward 2 | TM | |
| 200 | LI:332161.1:2000SEP08 | 1340 | 1393 | forward 2 | TM | |
| 200 | LI:332161.1:2000SEP08 | 1493 | 1579 | forward 2 | TM | |
| 200 | LI:332161.1:2000SEP08 | 1637 | 1717 | forward 2 | TM | |
| 200 | LI:332161.1:2000SEP08 | 1880 | 1966 | forward 2 | TM | |
| 200 | LI:332161.1:2000SEP08 | 2075 | 2161 | forward 2 | TM | |
| 200 | LI:332161.1:2000SEP08 | 2744 | 2818 | forward 2 | TM | |
| 200 | LI:332161.1:2000SEP08 | 258 | 311 | forward 3 | TM | N in |
| 200 | LI:332161.1:2000SEP08 | 1524 | 1610 | forward 3 | TM | N in |
| 200 | LI:332161.1:2000SEP08 | 2025 | 2111 | forward 3 | TM | N in |
| 200 | LI:332161.1:2000SEP08 | 2289 | 2375 | forward 3 | TM | N in |
| 201 | LI:184867.1:2000SEP08 | 1985 | 2041 | forward 2 | TM | N in |
| 201 | LI:184867.1:2000SEP08 | 771 | 854 | forward 3 | TM | N out |
| 202 | LI:229932.4:2000SEP08 | 76 | 162 | forward 1 | TM | N out |
| 202 | LI:229932.4:2000SEP08 | 229 | 300 | forward 1 | TM | N out |
| 202 | LI:229932.4:2000SEP08 | 1249 | 1329 | forward 1 | TM | N out |
| 202 | LI:229932.4:2000SEP08 | 1438 | 1524 | forward 1 | TM | N out |
| 202 | LI:229932.4:2000SEP08 | 1678 | 1764 | forward 1 | TM | N out |
| 202 | LI:229932.4:2000SEP08 | 68 | 142 | forward 2 | TM | N out |
| 202 | LI:229932.4:2000SEP08 | 215 | 271 | forward 2 | TM | N out |
| 202 | LI:229932.4:2000SEP08 | 734 | 820 | forward 2 | TM | N out |
| 202 | LI:229932.4:2000SEP08 | 1220 | 1291 | forward 2 | TM | N out |
| 202 | LI:229932.4:2000SEP08 | 1565 | 1618 | forward 2 | TM | N out |
| 202 | LI:229932.4:2000SEP08 | 60 | 146 | forward 3 | TM | |
| 202 | LI:229932.4:2000SEP08 | 348 | 425 | forward 3 | TM | |
| 202 | LI:229932.4:2000SEP08 | 762 | 848 | forward 3 | TM | |
| 202 | LI:229932.4:2000SEP08 | 1239 | 1325 | forward 3 | TM | |
| 202 | LI:229932.4:2000SEP08 | 1401 | 1487 | forward 3 | TM | |
| 202 | LI:229932.4:2000SEP08 | 1629 | 1703 | forward 3 | TM | |
| 203 | LI:1189932.1:2000SEP08 | 277 | 363 | forward 1 | TM | N out |
| 203 | LI:1189932.1:2000SEP08 | 1141 | 1203 | forward 1 | TM | N out |
| 203 | LI:1189932.1:2000SEP08 | 1216 | 1278 | forward 1 | TM | N out |
| 203 | LI:1189932.1:2000SEP08 | 1411 | 1497 | forward 1 | TM | N out |
| 203 | LI:1189932.1:2000SEP08 | 1642 | 1707 | forward 1 | TM | N out |
| 203 | LI:1189932.1:2000SEP08 | 1795 | 1881 | forward 1 | TM | N out |
| 203 | LI:1189932.1:2000SEP08 | 2125 | 2211 | forward 1 | TM | N out |
| 203 | LI:1189932.1:2000SEP08 | 89 | 151 | forward 2 | TM | N in |
| 203 | LI:1189932.1:2000SEP08 | 218 | 304 | forward 2 | TM | N in |
| 203 | LI:1189932.1:2000SEP08 | 320 | 388 | forward 2 | TM | N in |
| 203 | LI:1189932.1:2000SEP08 | 386 | 451 | forward 2 | TM | N in |
| 203 | LI:1189932.1:2000SEP08 | 572 | 643 | forward 2 | TM | N in |
| 203 | LI:1189932.1:2000SEP08 | 1142 | 1222 | forward 2 | TM | N in |
| 203 | LI:1189932.1:2000SEP08 | 1412 | 1498 | forward 2 | TM | N in |
| 203 | LI:1189932.1:2000SEP08 | 1928 | 1990 | forward 2 | TM | N in |
| 203 | LI:1189932.1:2000SEP08 | 2021 | 2107 | forward 2 | TM | N in |
| 203 | LI:1189932.1:2000SEP08 | 2141 | 2227 | forward 2 | TM | N in |
| 203 | LI:1189932.1:2000SEP08 | 522 | 608 | forward 3 | TM | N out |
| 203 | LI:1189932.1:2000SEP08 | 858 | 944 | forward 3 | TM | N out |
| 203 | LI:1189932.1:2000SEP08 | 1161 | 1247 | forward 3 | TM | N out |
| 203 | LI:1189932.1:2000SEP08 | 1992 | 2066 | forward 3 | TM | N out |
| 203 | LI:1189932.1:2000SEP08 | 2139 | 2210 | forward 3 | TM | N out |
| 204 | LI:1076689.1:2000SEP08 | 43 | 114 | forward 1 | TM | N in |
| 204 | LI:1076689.1:2000SEP08 | 326 | 373 | forward 2 | TM | N in |
| 205 | LI:415181.2:2000SEP08 | 1033 | 1113 | forward 1 | TM | N in |
| 205 | LI:415181.2:2000SEP08 | 1363 | 1434 | forward 1 | TM | N in |
| 205 | LI:415181.2:2000SEP08 | 914 | 1000 | forward 2 | TM | |
| 206 | LI:296358.1:2000SEP08 | 760 | 819 | forward 1 | TM | N out |
| 206 | LI:296358.1:2000SEP08 | 997 | 1068 | forward 1 | TM | N out |
| 206 | LI:296358.1:2000SEP08 | 1147 | 1233 | forward 1 | TM | N out |
| 206 | LI:296358.1:2000SEP08 | 197 | 247 | forward 2 | TM | N out |
| 206 | LI:296358.1:2000SEP08 | 356 | 442 | forward 2 | TM | N out |
| 207 | LI:205186.3:2000SEP08 | 644 | 697 | forward 2 | TM | N in |
| 207 | LI:205186.3:2000SEP08 | 812 | 877 | forward 2 | TM | N in |
| 208 | LI:220537.2:2000SEP08 | 595 | 681 | forward 1 | TM | N in |
| 208 | LI:220537.2:2000SEP08 | 1429 | 1515 | forward 1 | TM | N in |
| 208 | LI:220537.2:2000SEP08 | 245 | 331 | forward 2 | TM | N out |
| 208 | LI:220537.2:2000SEP08 | 362 | 415 | forward 2 | TM | N out |
| 208 | LI:220537.2:2000SEP08 | 1058 | 1129 | forward 2 | TM | N out |
| 208 | LI:220537.2:2000SEP08 | 1136 | 1222 | forward 2 | TM | N out |
| 208 | LI:220537.2:2000SEP08 | 1472 | 1555 | forward 2 | TM | N out |
| 208 | LI:220537.2:2000SEP08 | 1820 | 1897 | forward 2 | TM | N out |
| 208 | LI:220537.2:2000SEP08 | 216 | 299 | forward 3 | TM | N out |
| 208 | LI:220537.2:2000SEP08 | 447 | 509 | forward 3 | TM | N out |
| 208 | LI:220537.2:2000SEP08 | 522 | 584 | forward 3 | TM | N out |
| 208 | LI:220537.2:2000SEP08 | 624 | 686 | forward 3 | TM | N out |
| 208 | LI:220537.2:2000SEP08 | 702 | 764 | forward 3 | TM | N out |
| 208 | LI:220537.2:2000SEP08 | 1467 | 1553 | forward 3 | TM | N out |
| 208 | LI:220537.2:2000SEP08 | 1806 | 1892 | forward 3 | TM | N out |
| 209 | LI:248364.2:2000SEP08 | 439 | 495 | forward 1 | TM | N out |
| 210 | LI:2048338.1:2000SEP08 | 268 | 354 | forward 1 | TM | N in |
| 210 | LI:2048338.1:2000SEP08 | 385 | 459 | forward 1 | TM | N in |
| 210 | LI:2048338.1:2000SEP08 | 541 | 591 | forward 1 | TM | N in |
| 210 | LI:2048338.1:2000SEP08 | 281 | 358 | forward 2 | TM | N in |
| 211 | LI:1185203.8:2000SEP08 | 73 | 135 | forward 1 | TM | N out |
| 211 | LI:1185203.8:2000SEP08 | 148 | 210 | forward 1 | TM | N out |
| 211 | LI:1185203.8:2000SEP08 | 226 | 294 | forward 1 | TM | N out |
| 212 | LI:021770.3:2000SEP08 | 593 | 673 | forward 2 | TM | N out |
| 212 | LI:021770.3:2000SEP08 | 348 | 434 | forward 3 | TM | N in |
| 212 | LI:021770.3:2000SEP08 | 462 | 539 | forward 3 | TM | N in |
| 212 | LI:021770.3:2000SEP08 | 564 | 641 | forward 3 | TM | N in |
| 212 | LI:021770.3:2000SEP08 | 861 | 944 | forward 3 | TM | N in |
| 213 | LI:1185841.1:2000SEP08 | 1132 | 1182 | forward 1 | TM | N out |
| 213 | LI:1185841.1:2000SEP08 | 2434 | 2520 | forward 1 | TM | N out |
| 213 | LI:1185841.1:2000SEP08 | 965 | 1030 | forward 2 | TM | N in |
| 213 | LI:1185841.1:2000SEP08 | 2381 | 2437 | forward 2 | TM | N in |
| 214 | LI:1181710.1:2000SEP08 | 221 | 307 | forward 2 | TM | N out |
| 215 | LI:2048959.1:2000SEP08 | 262 | 318 | forward 1 | TM | N out |
| 216 | LI:798494.1:2000SEP08 | 595 | 657 | forward 1 | TM | N out |
| 216 | LI:798494.1:2000SEP08 | 670 | 732 | forward 1 | TM | N out |
| 216 | LI:798494.1:2000SEP08 | 215 | 268 | forward 2 | TM | N out |
| 216 | LI:798494.1:2000SEP08 | 12 | 62 | forward 3 | TM | N out |
| 216 | LI:798494.1:2000SEP08 | 60 | 113 | forward 3 | TM | N out |
| 217 | LI:2049223.1:2000SEP08 | 436 | 507 | forward 1 | TM | N in |
| 217 | LI:2049223.1:2000SEP08 | 416 | 484 | forward 2 | TM | N out |
| 218 | LI:1177833.1:2000SEP08 | 712 | 798 | forward 1 | TM | N in |
| 219 | LI:2049267.1:2000SEP08 | 215 | 289 | forward 2 | TM | N out |
| 219 | LI:2049267.1:2000SEP08 | 240 | 323 | forward 3 | TM | N out |
| 220 | LI:1165939.1:2000SEP08 | 554 | 640 | forward 2 | TM | |
| 221 | LI:1170958.1:2000SEP08 | 326 | 376 | forward 2 | TM | N out |
| 222 | LI:1089827.1:2000SEP08 | 1507 | 1584 | forward 1 | TM | N in |
| 223 | LI:792112.1:2000SEP08 | 818 | 892 | forward 2 | TM | N in |
| 223 | LI:792112.1:2000SEP08 | 9 | 86 | forward 3 | TM | |
| 223 | LI:792112.1:2000SEP08 | 669 | 755 | forward 3 | TM | |
| 223 | LI:792112.1:2000SEP08 | 783 | 860 | forward 3 | TM | |
| 224 | LI:282219.2:2000SEP08 | 661 | 732 | forward 1 | TM | N out |
| 224 | LI:282219.2:2000SEP08 | 491 | 577 | forward 2 | TM | N in |
| 224 | LI:282219.2:2000SEP08 | 659 | 745 | forward 2 | TM | N in |
| 224 | LI:282219.2:2000SEP08 | 408 | 458 | forward 3 | TM | N in |
| 225 | LI:1088010.2:2000SEP08 | 289 | 375 | forward 1 | TM | N in |
| 225 | LI:1088010.2:2000SEP08 | 716 | 793 | forward 2 | TM | N in |
| 225 | LI:1088010.2:2000SEP08 | 1229 | 1315 | forward 2 | TM | N in |
| 225 | LI:1088010.2:2000SEP08 | 903 | 980 | forward 3 | TM | N out |
| 225 | LI:1088010.2:2000SEP08 | 1002 | 1088 | forward 3 | TM | N out |
| 225 | LI:1088010.2:2000SEP08 | 1185 | 1247 | forward 3 | TM | N out |
| 225 | LI:1088010.2:2000SEP08 | 1272 | 1334 | forward 3 | TM | N out |
| 226 | LI:1165276.1:2000SEP08 | 301 | 363 | forward 1 | TM | N in |
| 226 | LI:1165276.1:2000SEP08 | 882 | 968 | forward 3 | TM | N out |
| 227 | LI:1169524.2:2000SEP08 | 623 | 709 | forward 2 | TM | |
| 227 | LI:1169524.2:2000SEP08 | 713 | 778 | forward 2 | TM | |
| 227 | LI:1169524.2:2000SEP08 | 842 | 928 | forward 2 | TM | |
| 228 | LI:1180255.1:2000SEP08 | 731 | 784 | forward 2 | TM | N in |
| 228 | LI:1180255.1:2000SEP08 | 872 | 931 | forward 2 | TM | N in |
| 228 | LI:1180255.1:2000SEP08 | 1199 | 1285 | forward 2 | TM | N in |
| 229 | LI:1091903.1:2000SEP08 | 454 | 540 | forward 1 | TM | N out |
| 230 | LI:1169219.1:2000SEP08 | 496 | 582 | forward 1 | TM | N in |
| 230 | LI:1169219.1:2000SEP08 | 509 | 595 | forward 2 | TM | N out |
| 230 | LI:1169219.1:2000SEP08 | 495 | 581 | forward 3 | TM | |
| 231 | LI:2050313.1:2000SEP08 | 2605 | 2664 | forward 1 | TM | N in |
| 231 | LI:2050313.1:2000SEP08 | 3229 | 3315 | forward 1 | TM | N in |
| 231 | LI:2050313.1:2000SEP08 | 3412 | 3498 | forward 1 | TM | N in |
| 231 | LI:2050313.1:2000SEP08 | 1886 | 1954 | forward 2 | TM | N in |
| 231 | LI:2050313.1:2000SEP08 | 2654 | 2731 | forward 2 | TM | N in |
| 231 | LI:2050313.1:2000SEP08 | 2924 | 2986 | forward 2 | TM | N in |
| 231 | LI:2050313.1:2000SEP08 | 3029 | 3091 | forward 2 | TM | N in |
| 231 | LI:2050313.1:2000SEP08 | 3482 | 3568 | forward 2 | TM | N in |
| 231 | LI:2050313.1:2000SEP08 | 855 | 920 | forward 3 | TM | N out |
| 231 | LI:2050313.1:2000SEP08 | 2445 | 2531 | forward 3 | TM | N out |
| 231 | LI:2050313.1:2000SEP08 | 2646 | 2708 | forward 3 | TM | N out |
| 231 | LI:2050313.1:2000SEP08 | 2721 | 2783 | forward 3 | TM | N out |
| 231 | LI:2050313.1:2000SEP08 | 3114 | 3170 | forward 3 | TM | N out |
| 231 | LI:2050313.1:2000SEP08 | 3504 | 3590 | forward 3 | TM | N out |
| 232 | LI:209351.3:2000SEP08 | 586 | 645 | forward 1 | TM | N in |
| 232 | LI:209351.3:2000SEP08 | 1945 | 2025 | forward 1 | TM | N in |
| 232 | LI:209351.3:2000SEP08 | 353 | 415 | forward 2 | TM | |
| 232 | LI:209351.3:2000SEP08 | 665 | 718 | forward 2 | TM | |
| 232 | LI:209351.3:2000SEP08 | 1313 | 1387 | forward 2 | TM | |
| 232 | LI:209351.3:2000SEP08 | 1991 | 2041 | forward 2 | TM | |
| 232 | LI:209351.3:2000SEP08 | 2153 | 2230 | forward 2 | TM | |
| 232 | LI:209351.3:2000SEP08 | 2390 | 2443 | forward 2 | TM | |
| 232 | LI:209351.3:2000SEP08 | 801 | 869 | forward 3 | TM | N in |
| 232 | LI:209351.3:2000SEP08 | 1926 | 2006 | forward 3 | TM | N in |
| 232 | LI:209351.3:2000SEP08 | 2382 | 2456 | forward 3 | TM | N in |
| 233 | LI:119900.1:2000SEP08 | 667 | 744 | forward 1 | TM | N in |
| 233 | LI:119900.1:2000SEP08 | 521 | 607 | forward 2 | TM | N out |
| 233 | LI:119900.1:2000SEP08 | 719 | 799 | forward 2 | TM | N out |
| 233 | LI:119900.1:2000SEP08 | 24 | 98 | forward 3 | TM | |
| 233 | LI:119900.1:2000SEP08 | 678 | 764 | forward 3 | TM | |
| 234 | LI:2052274.1:2000SEP08 | 332 | 418 | forward 2 | TM | N in |
| 234 | LI:2052274.1:2000SEP08 | 1526 | 1612 | forward 2 | TM | N in |
| 234 | LI:2052274.1:2000SEP08 | 939 | 1025 | forward 3 | TM | N in |
| 234 | LI:2052274.1:2000SEP08 | 1500 | 1586 | forward 3 | TM | N in |
| 235 | LI:1075502.1:2000SEP08 | 200 | 277 | forward 2 | TM | N in |
| 235 | LI:1075502.1:2000SEP08 | 483 | 569 | forward 3 | TM | N in |
| 236 | LI:813697.1:2000SEP08 | 805 | 867 | forward 1 | TM | |
| 236 | LI:813697.1:2000SEP08 | 224 | 283 | forward 2 | TM | |
| 236 | LI:813697.1:2000SEP08 | 1638 | 1715 | forward 3 | TM | N out |
| 236 | LI:813697.1:2000SEP08 | 1722 | 1808 | forward 3 | TM | N out |
| 237 | LI:814261.1:2000SEP08 | 562 | 618 | forward 1 | TM | |
| 237 | LI:814261.1:2000SEP08 | 287 | 343 | forward 2 | TM | N out |
| 237 | LI:814261.1:2000SEP08 | 512 | 598 | forward 2 | TM | N out |
| 237 | LI:814261.1:2000SEP08 | 279 | 356 | forward 3 | TM | N out |
| 237 | LI:814261.1:2000SEP08 | 474 | 557 | forward 3 | TM | N out |
| 238 | LI:775334.1:2000SEP08 | 898 | 984 | forward 1 | TM | N in |
| 238 | LI:775334.1:2000SEP08 | 581 | 661 | forward 2 | TM | N out |
| 239 | LI:1180325.1:2000SEP08 | 833 | 919 | forward 2 | TM | N in |
| 240 | LI:1183147.3:2000SEP08 | 268 | 342 | forward 1 | TM | N in |
| 240 | LI:1183147.3:2000SEP08 | 370 | 456 | forward 1 | TM | N in |
| 240 | LI:1183147.3:2000SEP08 | 712 | 798 | forward 1 | TM | N in |
| 240 | LI:1183147.3:2000SEP08 | 937 | 1023 | forward 1 | TM | N in |
| 240 | LI:1183147.3:2000SEP08 | 374 | 460 | forward 2 | TM | N in |
| 240 | LI:1183147.3:2000SEP08 | 800 | 871 | forward 2 | TM | N in |
| 240 | LI:1183147.3:2000SEP08 | 968 | 1045 | forward 2 | TM | N in |
| 240 | LI:1183147.3:2000SEP08 | 1385 | 1450 | forward 2 | TM | N in |
| 240 | LI:1183147.3:2000SEP08 | 156 | 224 | forward 3 | TM | N out |
| 240 | LI:1183147.3:2000SEP08 | 285 | 347 | forward 3 | TM | N out |
| 240 | LI:1183147.3:2000SEP08 | 414 | 500 | forward 3 | TM | N out |
| 240 | LI:1183147.3:2000SEP08 | 687 | 770 | forward 3 | TM | N out |
| 240 | LI:1183147.3:2000SEP08 | 846 | 932 | forward 3 | TM | N out |
| 240 | LI:1183147.3:2000SEP08 | 1002 | 1067 | forward 3 | TM | N out |
| 241 | LI:1175373.3:2000SEP08 | 283 | 357 | forward 1 | TM | N out |
| 242 | LI:813757.1:2000SEP08 | 744 | 827 | forward 3 | TM | N in |
| 243 | LI:1182979.2:2000SEP08 | 370 | 456 | forward 1 | TM | N in |
| 243 | LI:1182979.2:2000SEP08 | 742 | 828 | forward 1 | TM | N in |
| 243 | LI:1182979.2:2000SEP08 | 705 | 767 | forward 3 | TM | N out |
| 243 | LI:1182979.2:2000SEP08 | 783 | 845 | forward 3 | TM | N out |
| 244 | LI:1177823.2:2000SEP08 | 307 | 393 | forward 1 | TM | N out |
| 244 | LI:1177823.2:2000SEP08 | 311 | 370 | forward 2 | TM | N in |
| 245 | LI:1174279.1:2000SEP08 | 634 | 720 | forward 1 | TM | N out |
| 245 | LI:1174279.1:2000SEP08 | 724 | 789 | forward 1 | TM | N out |
| 246 | LI:1178411.1:2000SEP08 | 1357 | 1443 | forward 1 | TM | |
| 246 | LI:1178411.1:2000SEP08 | 1290 | 1376 | forward 3 | TM | N in |
| 246 | LI:1178411.1:2000SEP08 | 1419 | 1472 | forward 3 | TM | N in |
| 247 | LI:1182739.1:2000SEP08 | 853 | 939 | forward 1 | TM | N in |
| 247 | LI:1182739.1:2000SEP08 | 1900 | 1986 | forward 1 | TM | N in |
| 247 | LI:1182739.1:2000SEP08 | 2131 | 2217 | forward 1 | TM | N in |
| 247 | LI:1182739.1:2000SEP08 | 2341 | 2403 | forward 1 | TM | N in |
| 247 | LI:1182739.1:2000SEP08 | 2446 | 2508 | forward 1 | TM | N in |
| 247 | LI:1182739.1:2000SEP08 | 803 | 859 | forward 2 | TM | N in |
| 247 | LI:1182739.1:2000SEP08 | 1268 | 1351 | forward 2 | TM | N in |
| 247 | LI:1182739.1:2000SEP08 | 1445 | 1513 | forward 2 | TM | N in |
| 247 | LI:1182739.1:2000SEP08 | 1796 | 1882 | forward 2 | TM | N in |
| 247 | LI:1182739.1:2000SEP08 | 2090 | 2176 | forward 2 | TM | N in |
| 247 | LI:1182739.1:2000SEP08 | 2267 | 2323 | forward 2 | TM | N in |
| 247 | LI:1182739.1:2000SEP08 | 2363 | 2449 | forward 2 | TM | N in |
| 247 | LI:1182739.1:2000SEP08 | 1182 | 1244 | forward 3 | TM | N out |
| 247 | LI:1182739.1:2000SEP08 | 1254 | 1316 | forward 3 | TM | N out |
| 247 | LI:1182739.1:2000SEP08 | 2100 | 2156 | forward 3 | TM | N out |
| 247 | LI:1182739.1:2000SEP08 | 2301 | 2387 | forward 3 | TM | N out |
| 248 | LI:234937.4:2000SEP08 | 301 | 387 | forward 1 | TM | N out |
| 249 | LI:1170660.1:2000SEP08 | 604 | 690 | forward 1 | TM | N in |
| 249 | LI:1170660.1:2000SEP08 | 757 | 843 | forward 1 | TM | N in |
| 249 | LI:1170660.1:2000SEP08 | 937 | 990 | forward 1 | TM | N in |
| 249 | LI:1170660.1:2000SEP08 | 1111 | 1179 | forward 1 | TM | N in |
| 249 | LI:1170660.1:2000SEP08 | 512 | 583 | forward 2 | TM | |
| 249 | LI:1170660.1:2000SEP08 | 911 | 973 | forward 2 | TM | |
| 249 | LI:1170660.1:2000SEP08 | 986 | 1048 | forward 2 | TM | |
| 249 | LI:1170660.1:2000SEP08 | 252 | 323 | forward 3 | TM | N out |
| 249 | LI:1170660.1:2000SEP08 | 591 | 677 | forward 3 | TM | N out |
| 249 | LI:1170660.1:2000SEP08 | 774 | 860 | forward 3 | TM | N out |
| 250 | LI:1144409.1:2000SEP08 | 25 | 81 | forward 1 | TM | N out |
| 250 | LI:1144409.1:2000SEP08 | 1708 | 1782 | forward 1 | TM | N out |
| 250 | LI:1144409.1:2000SEP08 | 2281 | 2367 | forward 1 | TM | N out |
| 250 | LI:1144409.1:2000SEP08 | 1658 | 1744 | forward 2 | TM | N in |
| 250 | LI:1144409.1:2000SEP08 | 1880 | 1927 | forward 2 | TM | N in |
| 250 | LI:1144409.1:2000SEP08 | 2294 | 2380 | forward 2 | TM | N in |
| 250 | LI:1144409.1:2000SEP08 | 1614 | 1697 | forward 3 | TM | N in |
| 250 | LI:1144409.1:2000SEP08 | 2217 | 2303 | forward 3 | TM | N in |
| 251 | LI:246290.10:2000SEP08 | 248 | 304 | forward 2 | TM | N out |
| 251 | LI:246290.10:2000SEP08 | 641 | 727 | forward 2 | TM | N out |
| 251 | LI:246290.10:2000SEP08 | 3 | 89 | forward 3 | TM | N out |
| 252 | LI:280034.1:2000SEP08 | 279 | 365 | forward 3 | TM | N out |

TABLE 5

| SEQ ID NO: | Template ID | Component ID | Start | Stop |
|---|---|---|---|---|
| 1 | LG:150318.1:2000SEP08 | 482540R6 | 1 | 350 |
| 1 | LG:150318.1:2000SEP08 | 482540H1 | 1 | 238 |
| 1 | LG:150318.1:2000SEP08 | 6021134T8 | 126 | 531 |
| 1 | LG:150318.1:2000SEP08 | 6021134R8 | 126 | 537 |
| 1 | LG:150318.1:2000SEP08 | 6021134H1 | 126 | 629 |
| 2 | LG:022529.1:2000SEP08 | 7593029H1 | 1 | 521 |
| 2 | LG:022529.1:2000SEP08 | g1645695 | 112 | 517 |
| 2 | LG:022529.1:2000SEP08 | 2160267H1 | 275 | 514 |
| 2 | LG:022529.1:2000SEP08 | 5284828H1 | 309 | 583 |
| 2 | LG:022529.1:2000SEP08 | g1880347 | 313 | 601 |
| 2 | LG:022529.1:2000SEP08 | 7352207H1 | 330 | 707 |
| 2 | LG:022529.1:2000SEP08 | 6863346H1 | 339 | 830 |
| 2 | LG:022529.1:2000SEP08 | 3351341H1 | 423 | 681 |
| 2 | LG:022529.1:2000SEP08 | 077299H1 | 432 | 632 |
| 2 | LG:022529.1:2000SEP08 | 078146H1 | 438 | 646 |
| 2 | LG:022529.1:2000SEP08 | 3801790F6 | 523 | 1030 |
| 2 | LG:022529.1:2000SEP08 | 3801790H1 | 523 | 823 |
| 2 | LG:022529.1:2000SEP08 | 3769238F6 | 523 | 1043 |
| 2 | LG:022529.1:2000SEP08 | 3802590H1 | 524 | 810 |
| 2 | LG:022529.1:2000SEP08 | 6217218H1 | 549 | 1039 |
| 2 | LG:022529.1:2000SEP08 | g5743872 | 664 | 1137 |
| 2 | LG:022529.1:2000SEP08 | g3051534 | 685 | 1047 |
| 2 | LG:022529.1:2000SEP08 | g4195758 | 789 | 1045 |
| 3 | LG:352559.1:2000SEP08 | 6453567H1 | 1 | 503 |
| 3 | LG:352559.1:2000SEP08 | 4052122H1 | 185 | 457 |
| 3 | LG:352559.1:2000SEP08 | 4052122F7 | 185 | 636 |
| 3 | LG:352559.1:2000SEP08 | g3897399 | 255 | 371 |
| 4 | LG:175223.1:2000SEP08 | 3081155F6 | 1 | 417 |
| 4 | LG:175223.1:2000SEP08 | 3081155H1 | 2 | 315 |
| 4 | LG:175223.1:2000SEP08 | 3081155T6 | 31 | 465 |
| 4 | LG:175223.1:2000SEP08 | g1648354 | 58 | 457 |
| 4 | LG:175223.1:2000SEP08 | 5800009H1 | 99 | 507 |
| 5 | LG:476989.1:2000SEP08 | 6874941H1 | 1 | 169 |
| 5 | LG:476989.1:2000SEP08 | 6874955H1 | 1 | 204 |
| 5 | LG:476989.1:2000SEP08 | g4393499 | 51 | 432 |
| 5 | LG:476989.1:2000SEP08 | g4190810 | 96 | 432 |
| 6 | LG:253268.7:2000SEP08 | 6772515H1 | 607 | 1083 |
| 6 | LG:253268.7:2000SEP08 | g5526194 | 596 | 941 |
| 6 | LG:253268.7:2000SEP08 | g4729197 | 690 | 941 |
| 6 | LG:253268.7:2000SEP08 | g5809986 | 501 | 941 |
| 6 | LG:253268.7:2000SEP08 | g2540947 | 535 | 937 |
| 6 | LG:253268.7:2000SEP08 | g922062 | 586 | 935 |
| 6 | LG:253268.7:2000SEP08 | 6772515J1 | 454 | 961 |
| 6 | LG:253268.7:2000SEP08 | 2535554F6 | 493 | 929 |
| 6 | LG:253268.7:2000SEP08 | g4392225 | 520 | 923 |
| 6 | LG:253268.7:2000SEP08 | g3424830 | 561 | 923 |
| 6 | LG:253268.7:2000SEP08 | g4533991 | 464 | 923 |
| 6 | LG:253268.7:2000SEP08 | g885146 | 693 | 914 |
| 6 | LG:253268.7:2000SEP08 | g917418 | 564 | 914 |
| 6 | LG:253268.7:2000SEP08 | g888754 | 605 | 914 |
| 6 | LG:253268.7:2000SEP08 | g922196 | 606 | 913 |
| 6 | LG:253268.7:2000SEP08 | g888391 | 595 | 912 |
| 6 | LG:253268.7:2000SEP08 | g5364213 | 457 | 897 |
| 6 | LG:253268.7:2000SEP08 | g990349 | 560 | 886 |
| 6 | LG:253268.7:2000SEP08 | g888707 | 589 | 884 |
| 6 | LG:253268.7:2000SEP08 | g885121 | 574 | 884 |
| 6 | LG:253268.7:2000SEP08 | g888978 | 669 | 883 |
| 6 | LG:253268.7:2000SEP08 | g990350 | 535 | 881 |
| 6 | LG:253268.7:2000SEP08 | 6852955H1 | 306 | 854 |
| 6 | LG:253268.7:2000SEP08 | 7236518H1 | 249 | 789 |
| 6 | LG:253268.7:2000SEP08 | 2535554H1 | 493 | 737 |
| 6 | LG:253268.7:2000SEP08 | g889930 | 353 | 601 |
| 6 | LG:253268.7:2000SEP08 | g888417 | 353 | 587 |
| 6 | LG:253268.7:2000SEP08 | 5773584H1 | 69 | 569 |
| 6 | LG:253268.7:2000SEP08 | 488757R1 | 1 | 536 |
| 7 | LG:401322.1:2000SEP08 | 4875149T6 | 344 | 737 |
| 7 | LG:401322.1:2000SEP08 | g3202357 | 457 | 784 |
| 7 | LG:401322.1:2000SEP08 | 748032H1 | 574 | 776 |
| 7 | LG:401322.1:2000SEP08 | 750543H1 | 1 | 183 |
| 7 | LG:401322.1:2000SEP08 | g1474315 | 42 | 323 |
| 7 | LG:401322.1:2000SEP08 | 3722596H1 | 61 | 333 |
| 7 | LG:401322.1:2000SEP08 | 4875149F6 | 70 | 195 |
| 7 | LG:401322.1:2000SEP08 | 4875149H1 | 70 | 257 |
| 7 | LG:401322.1:2000SEP08 | 6561179H1 | 71 | 633 |
| 7 | LG:401322.1:2000SEP08 | 2029094R6 | 110 | 485 |
| 7 | LG:401322.1:2000SEP08 | 2029094H1 | 121 | 372 |
| 7 | LG:401322.1:2000SEP08 | 5950410H1 | 125 | 401 |
| 7 | LG:401322.1:2000SEP08 | 5950630H1 | 125 | 405 |
| 7 | LG:401322.1:2000SEP08 | g4998660 | 180 | 478 |
| 7 | LG:401322.1:2000SEP08 | 4933727H1 | 239 | 384 |
| 7 | LG:401322.1:2000SEP08 | 3533254H1 | 273 | 564 |
| 8 | LG:1328436.1:2000SEP08 | 2505871H1 | 1 | 210 |
| 8 | LG:1328436.1:2000SEP08 | 6200837H1 | 4 | 346 |
| 8 | LG:1328436.1:2000SEP08 | 6201568T8 | 26 | 343 |
| 8 | LG:1328436.1:2000SEP08 | 6201568H1 | 26 | 629 |
| 8 | LG:1328436.1:2000SEP08 | 6201568F8 | 26 | 211 |
| 8 | LG:1328436.1:2000SEP08 | g2630885 | 36 | 432 |
| 8 | LG:1328436.1:2000SEP08 | g723429 | 7 | 191 |
| 8 | LG:1328436.1:2000SEP08 | 6200837T8 | 76 | 343 |
| 8 | LG:1328436.1:2000SEP08 | g725043 | 91 | 362 |
| 8 | LG:1328436.1:2000SEP08 | g725147 | 312 | 572 |
| 9 | LG:475404.1:2000SEP08 | 460544H1 | 20 | 245 |
| 9 | LG:475404.1:2000SEP08 | 5998240H1 | 1 | 484 |
| 9 | LG:475404.1:2000SEP08 | 5998240T8 | 1 | 408 |
| 9 | LG:475404.1:2000SEP08 | 5998240F8 | 1 | 398 |
| 9 | LG:475404.1:2000SEP08 | 455966H1 | 20 | 255 |
| 9 | LG:475404.1:2000SEP08 | 455162H1 | 20 | 272 |
| 9 | LG:475404.1:2000SEP08 | 455162R6 | 20 | 521 |
| 9 | LG:475404.1:2000SEP08 | 458061H1 | 20 | 259 |
| 9 | LG:475404.1:2000SEP08 | 460544R6 | 20 | 326 |
| 9 | LG:475404.1:2000SEP08 | 3206542H1 | 25 | 202 |
| 9 | LG:475404.1:2000SEP08 | 4663964H1 | 37 | 289 |
| 9 | LG:475404.1:2000SEP08 | 7652675J1 | 47 | 172 |
| 9 | LG:475404.1:2000SEP08 | g1807165 | 81 | 278 |
| 9 | LG:475404.1:2000SEP08 | g2401959 | 129 | 486 |
| 9 | LG:475404.1:2000SEP08 | 3614680H1 | 155 | 272 |
| 9 | LG:475404.1:2000SEP08 | g5101146 | 180 | 486 |
| 10 | LG:1384132.1:2000SEP08 | 5754964H1 | 1 | 453 |
| 10 | LG:1384132.1:2000SEP08 | g5366771 | 73 | 480 |
| 10 | LG:1384132.1:2000SEP08 | 3246016H1 | 198 | 446 |
| 11 | LG:410804.18:2000SEP08 | 3270045H1 | 1 | 252 |
| 12 | LG:1082306.1:2000SEP08 | 2590822H1 | 60 | 224 |
| 12 | LG:1082306.1:2000SEP08 | 2875044H1 | 13 | 269 |
| 12 | LG:1082306.1:2000SEP08 | 5944268H1 | 12 | 288 |
| 12 | LG:1082306.1:2000SEP08 | 908479H1 | 39 | 241 |
| 12 | LG:1082306.1:2000SEP08 | 2972368F6 | 1 | 586 |
| 12 | LG:1082306.1:2000SEP08 | 2972368H1 | 1 | 292 |
| 12 | LG:1082306.1:2000SEP08 | 1831460H1 | 393 | 656 |
| 12 | LG:1082306.1:2000SEP08 | 92270170 | 247 | 638 |
| 12 | LG:1082306.1:2000SEP08 | 1732781H1 | 118 | 297 |
| 12 | LG:1082306.1:2000SEP08 | 4136083H1 | 590 | 657 |
| 12 | LG:1082306.1:2000SEP08 | 5152624H1 | 463 | 723 |
| 12 | LG:1082306.1:2000SEP08 | 2634451H1 | 434 | 685 |
| 13 | LG:233814.4:2000SEP08 | 6907331J1 | 757 | 1055 |
| 13 | LG:233814.4:2000SEP08 | 7435089H1 | 1 | 580 |
| 13 | LG:233814.4:2000SEP08 | 7579295H1 | 475 | 927 |
| 13 | LG:233814.4:2000SEP08 | 2818676H1 | 511 | 729 |
| 13 | LG:233814.4:2000SEP08 | 1943949H1 | 632 | 863 |
| 13 | LG:233814.4:2000SEP08 | 7403021H1 | 703 | 1146 |
| 14 | LG:977478.5:2000SEP08 | 7087230H1 | 1 | 549 |
| 14 | LG:977478.5:2000SEP08 | 5525655H2 | 50 | 293 |
| 14 | LG:977478.5:2000SEP08 | 7198649H1 | 74 | 395 |
| 14 | LG:977478.5:2000SEP08 | 3218939H1 | 164 | 284 |
| 14 | LG:977478.5:2000SEP08 | 2137947T7 | 182 | 714 |
| 14 | LG:977478.5:20003EP08 | 2137242H1 | 274 | 541 |
| 14 | LG:977478.5:2000SEP08 | 2970423T6 | 312 | 783 |
| 14 | LG:977478.5:2000SEP08 | 2970423F6 | 319 | 720 |
| 14 | LG:977478.5:2000SEP08 | 2970423H2 | 319 | 633 |
| 14 | LG:977478.5:2000SEP08 | g6464000 | 464 | 821 |
| 14 | LG:977478.5:2000SEP08 | 974781H1 | 501 | 774 |
| 14 | LG:977478.5:2000SEP08 | g6044798 | 530 | 821 |
| 15 | LG:025931.1:2000SEP08 | 6075128F6 | 1 | 524 |
| 15 | LG:025931.1:2000SEP08 | 7002695H1 | 272 | 846 |
| 15 | LG:025931.1:2000SEP08 | 1949002R6 | 435 | 707 |
| 15 | LG:025931.1:2000SEP08 | 1949002H1 | 435 | 692 |
| 15 | LG:025931.1:2000SEP08 | 96711855 | 473 | 929 |
| 15 | LG:025931.1:2000SEP08 | 3348048H1 | 497 | 748 |
| 15 | LG:025931.1:2000SEP08 | 7382758H1 | 573 | 962 |
| 15 | LG:025931.1:2000SEP08 | 5844022H1 | 224 | 474 |
| 15 | LG:025931.1:2000SEP08 | 5844022F6 | 223 | 614 |
| 15 | LG:025931.1:2000SEP08 | 6075128H1 | 1 | 185 |
| 16 | LG:885368.1:2000SEP08 | 590978217 | 1 | 494 |
| 16 | LG:885368.1:2000SEP08 | 6269652H1 | 1 | 415 |
| 16 | LG:885368.1:2000SEP08 | 5909782H1 | 1 | 306 |
| 16 | LG:885368.1:2000SEP08 | 5909782F7 | 1 | 578 |
| 16 | LG:885368.1:2000SEP08 | 6269652F8 | 1 | 682 |
| 16 | LG:885368.1:2000SEP08 | 5909795H1 | 1 | 305 |
| 16 | LG:885368.1:2000SEP08 | 5909895H1 | 1 | 133 |
| 16 | LG:885368.1:2000SEP08 | 6269652T8 | 99 | 618 |
| 16 | LG:885368.1:2000SEP08 | 4982679T6 | 377 | 550 |
| 17 | LG:1054900.1:2000SEP08 | 2327449H1 | 15 | 248 |
| 17 | LG:1054900.1:2000SEP08 | 2327457T6 | 1 364 | |
| 17 | LG:1054900.1:2000SEP08 | 2327449T6 | 1 288 | |
| 17 | LG:1054900.1:2000SEP08 | 2327449R6 | 13 | 408 |
| 17 | LG:1054900.1:2000SEP08 | 2327457R6 | 13 | 402 |
| 17 | LG:1054900.1:2000SEP08 | 6537441H1 | 147 | 499 |
| 17 | LG:1054900.1:2000SEP08 | 5108773H1 | 196 | 254 |
| 18 | LG:995186.2:2000SEP08 | 5056145F6 | 1 | 549 |
| 18 | LG:995186.2:2000SEP08 | 6061051F8 | 169 | 669 |
| 19 | LG:435048.23:2000SEP08 | 6867561H1 | 1 | 461 |
| 19 | LG:435048.23:2000SEP08 | 3531553H1 | 53 | 327 |
| 20 | LG:954859.1:2000SEP08 | g6196726 | 233 | 638 |
| 20 | LG:954859.1:2000SEP08 | g2344451 | 154 | 637 |
| 20 | LG:954859.1:2000SEP08 | 3814110T6 | 148 | 602 |
| 20 | LG:954859.1:2000SEP08 | 3815093T6 | 146 | 591 |
| 20 | LG:954859.1:2000SEP08 | 6475045H1 | 1 | 548 |
| 20 | LG:954859.1:2000SEP08 | 6413028H1 | 1 | 373 |
| 21 | LG:364370.1:2000SEP08 | 6798273H1 | 1 | 497 |
| 21 | LG:364370.1:2000SEP08 | 6798273F8 | 1 | 574 |
| 21 | LG:364370.1:2000SEP08 | 4431872H1 | 3 | 82 |
| 21 | LG:364370.1:2000SEP08 | 6792402T8 | 9 | 592 |
| 21 | LG:364370.1:2000SEP08 | 6792402F8 | 9 | 705 |
| 21 | LG:364370.1:2000SEP08 | 6792402H1 | 9 | 500 |
| 21 | LG:364370.1:2000SEP08 | 6798273T8 | 362 | 543 |
| 22 | LG:1098789.1:2000SEP08 | 3537002T6 | 1 | 461 |
| 23 | LG:201540.2:2000SEP08 | 5764854H1 | 2 | 545 |
| 23 | LG:201540.2:2000SEP08 | 4664068F6 | 1 | 316 |
| 23 | LG:201540.2:2000SEP08 | 4664068H1 | 2 | 237 |
| 23 | LG:201540.2:2000SEP08 | 5968685H1 | 26 | 592 |
| 23 | LG:201540.2:2000SEP08 | 3160094H1 | 98 | 362 |
| 23 | LG:201540.2:2000SEP08 | 652754H1 | 101 | 353 |
| 23 | LG:201540.2:2000SEP08 | 6788325H1 | 118 | 489 |
| 23 | LG:201540.2:2000SEP08 | g1950834 | 690 | 910 |
| 23 | LG:201540.2:2000SEP08 | 2739044F6 | 689 | 1171 |
| 23 | LG:201540.2:2000SEP08 | 6829490J1 | 703 | 1219 |
| 23 | LG:201540.2:2000SEP08 | 2739044H1 | 690 | 924 |
| 23 | LG:201540.2:2000SEP08 | 7328749H1 | 733 | 1261 |
| 23 | LG:201540.2:2000SEP08 | 6829490H1 | 712 | 1219 |
| 23 | LG:201540.2:2000SEP08 | 2857116H1 | 777 | 1049 |
| 23 | LG:201540.2:2000SEP08 | g769612 | 173 | 528 |
| 23 | LG:201540.2:2000SEP08 | 6490433H1 | 287 | 779 |
| 23 | LG:201540.2:2000SEP08 | 5096074T6 | 604 | 1006 |
| 23 | LG:201540.2:2000SEP08 | 7262169H1 | 642 | 1193 |
| 23 | LG:201540.2:2000SEP08 | 6310909H1 | 308 | 887 |
| 23 | LG:201540.2:2000SEP08 | 5096074H2 | 347 | 523 |
| 23 | LG:201540.2:2000SEP08 | 5096074F6 | 347 | 703 |
| 23 | LG:201540.2:2000SEP08 | 7701612J1 | 518 | 1093 |
| 23 | LG:201540.2:2000SEP08 | 4053114H1 | 100 | 386 |
| 23 | LG:201540.2:2000SEP08 | 4319192H1 | 131 | 397 |
| 23 | LG:201540.2:2000SEP08 | 3414632H1 | 132 | 252 |
| 23 | LG:201540.2:2000SEP08 | 7200283H1 | 142 | 699 |
| 24 | LG:1077357.1:2000SEP08 | 6245903F8 | 120 | 547 |
| 24 | LG:1077357.1:2000SEP08 | 6245903H1 | 121 | 532 |
| 24 | LG:1077357.1:2000SEP08 | g3250058 | 1 | 408 |
| 24 | LG:1077357.1:2000SEP08 | g3694145 | 26 | 408 |
| 24 | LG:1077357.1:2000SEP08 | 94333202 | 140 | 407 |
| 24 | LG:1077357.1:2000SEP08 | 6779728J1 | 1 | 370 |
| 25 | LG:1048846.4:2000SEP08 | 5433043T9 | 1 | 476 |
| 25 | LG:1048846.4:2000SEP08 | 3256037H1 | 191 | 435 |
| 25 | LG:1048846.4:2000SEP08 | 3256037R6 | 191 | 354 |
| 25 | LG:1048846.4:2000SEP08 | 3207387H1 | 213 | 461 |
| 25 | LG:1048846.4:2000SEP08 | 4316787H1 | 247 | 532 |
| 25 | LG:1048846.4:2000SEP08 | 4630864T8 | 263 | 483 |
| 26 | LG:336685.1:2000SEP08 | g1422427 | 85 | 461 |
| 26 | LG:336685.1:2000SEP08 | 3167506H1 | 1 | 292 |
| 26 | LG:336685.1:2000SEP08 | g3751213 | 82 | 477 |
| 26 | LG:336685.1:2000SEP08 | g3039675 | 93 | 477 |
| 26 | LG:336685.1:2000SEP08 | 7612160J1 | 631 | 1172 |
| 26 | LG:336685.1:2000SEP08 | 7585885H2 | 458 | 1146 |
| 26 | LG:336685.1:2000SEP08 | 7330357H1 | 428 | 965 |
| 26 | LG:336685.1:2000SEP08 | 7612160H1 | 246 | 869 |
| 26 | LG:336685.1:2000SEP08 | g3844561 | 389 | 808 |
| 26 | LG:336685.1:2000SEP08 | g2702908 | 347 | 805 |
| 26 | LG:336685.1:2000SEP08 | g1422428 | 441 | 805 |
| 27 | LG:1076253.1:2000SEP08 | 5677775H1 | 24 | 273 |
| 27 | LG:1076253.1:2000SEP08 | 828479H1 | 617 | 874 |
| 27 | LG:1076253.1:2000SEP08 | 5677775F6 | 24 | 610 |
| 27 | LG:1076253.1:2000SEP08 | 7392292H1 | 1 | 584 |
| 27 | LG:1076253.1:2000SEP08 | 4896201F8 | 298 | 724 |
| 27 | LG:1076253.1:2000SEP08 | 4896201H1 | 298 | 566 |
| 27 | LG:1076253.1:2000SEP08 | 7933083H1 | 389 | 898 |
| 27 | LG:1076253.1:2000SEP08 | 2717909H1 | 429 | 684 |
| 27 | LG:1076253.1:2000SEP08 | 392443R1 | 585 | 1112 |
| 27 | LG:1076253.1:2000SEP08 | 4896201T8 | 826 | 1314 |
| 27 | LG:1076253.1:2000SEP08 | 4896201T9 | 872 | 1357 |
| 27 | LG:1076253.1:2000SEP08 | 3989646T6 | 1029 | 1418 |
| 27 | LG:1076253.1:2000SEP08 | 2779195T6 | 1078 | 1411 |
| 27 | LG:1076253.1:2000SEP08 | 3989646R6 | 354 | 748 |
| 27 | LG:1076253.1:2000SEP08 | 3989646H1 | 354 | 512 |
| 28 | LG:1400601.2:2000SEP08 | 6967393H1 | 1 | 600 |
| 29 | LG:1079092.3:2000SEP08 | 6002889F8 | 1 | 660 |
| 29 | LG:1079092.3:2000SEP08 | 600288918 | 104 | 612 |
| 30 | LG:1086064.1:2000SEP08 | 456122R6 | 6 | 395 |
| 30 | LG:1086064.1:2000SEP08 | 5941956H1 | 8 | 286 |
| 30 | LG:1086064.1:2000SEP08 | 4727660F6 | 1 | 542 |
| 30 | LG:1086064.1:2000SEP08 | 7355369H1 | 1 | 526 |
| 30 | LG:1086064.1:2000SEP08 | 3730253H1 | 9 | 315 |
| 30 | LG:1086064.1:2000SEP08 | 1798649F6 | 6 | 361 |
| 30 | LG:1086064.1:2000SEP08 | 456122R1 | 6 | 583 |
| 30 | LG:1086064.1:2000SEP08 | 787063H1 | 16 | 134 |
| 30 | LG:1086064.1:2000SEP08 | 7354016H1 | 19 | 537 |
| 30 | LG:1086064.1:2000SEP08 | 456122F1 | 45 | 518 |
| 30 | LG:1086064.1:2000SEP08 | 3523404H 1 | 127 | 467 |
| 30 | LG:1086064.1:2000SEP08 | 456122T6 | 154 | 476 |
| 30 | LG:1086064.1:2000SEP08 | 1698965F6 | 165 | 529 |
| 30 | LG:1086064.1:2000SEP08 | 1698965T6 | 179 | 500 |
| 30 | LG:1086064.1:2000SEP08 | 2514834H1 | 16 | 323 |
| 30 | LG:1086064.1:2000SEP08 | 456180H1 | 6 | 246 |
| 30 | LG:1086064.1:2000SEP08 | 4549786H1 | 1 | 245 |
| 30 | LG:1086064.1:2000SEP08 | 4822628H1 | 2 | 283 |
| 30 | LG:1086064.1:2000SEP08 | 457138H1 | 6 | 149 |
| 30 | LG:1086064.1:2000SEP08 | 4727660H1 | 1 | 267 |
| 30 | LG:1086064.1:2000SEP08 | 458658H1 | 6 | 239 |
| 30 | LG:1086064.1:2000SEP08 | 461412H1 | 6 | 260 |
| 30 | LG:1086064.1:2000SEP08 | 456122H1 | 6 | 247 |
| 30 | LG:1086064.1:2000SEP08 | 460344H1 | 6 | 278 |
| 30 | LG:1086064.1:2000SEP08 | 460848H1 | 6 | 242 |
| 30 | LG:1086064.1:2000SEP08 | 1698965H1 | 165 | 396 |
| 30 | LG:1086064.1:2000SEP08 | 1322674H1 | 2 | 256 |
| 30 | LG:1086064.1:2000SEP08 | 460283H1 | 6 | 240 |
| 30 | LG:1086064.1:2000SEP08 | 3735303H1 | 22 | 340 |
| 30 | LG:1086064.1:2000SEP08 | 1798649H1 | 6 | 280 |
| 30 | LG:1086064.1:2000SEP08 | 1698730H1 | 165 | 359 |
| 31 | LG:1400608.1:2000SEP08 | 4399221H1 | 25 | 234 |
| 31 | LG:1400608.1:2000SEP08 | 5120882F6 | 1 | 353 |
| 31 | LG:1400608.1:2000SEP08 | 014670H1 | 7 | 288 |
| 31 | LG:1400608.1:2000SEP08 | 5378061H1 | 133 | 391 |
| 31 | LG:1400608.1:2000SEP08 | 5378061F6 | 133 | 591 |
| 31 | LG:1400608.1:2000SEP08 | g4265183 | 180 | 641 |
| 31 | LG:1400608.1:2000SEP08 | 4881456H1 | 274 | 353 |
| 31 | LG:1400608.1:2000SEP08 | 2882961H1 | 13 | 272 |
| 31 | LG:1400608.1:2000SEP08 | 5120882H1 | 1 | 265 |
| 31 | LG:1400608.1:2000SEP08 | 1899877H1 | 41 | 265 |
| 31 | LG:1400608.1:2000SEP08 | g1959316 | 41 | 357 |
| 32 | LG:399275.5:2000SEP08 | 2054642H1 | 354 | 539 |
| 32 | LG:399275.5:2000SEP08 | 7688287H1 | 1 | 410 |
| 32 | LG:399275.5:2000SEP08 | 1561014H1 | 169 | 351 |
| 32 | LG:399275.5:2000SEP08 | 7688287J1 | 192 | 657 |
| 33 | LG:293943.1:2000SEP08 | 7102403R8 | 1 | 647 |
| 33 | LG:293943.1:2000SEP08 | g2007300 | 100 | 453 |
| 33 | LG:293943.1:2000SEP08 | 642516117 | 254 | 362 |
| 33 | LG:293943.1:2000SEP08 | 6425161H1 | 254 | 537 |
| 33 | LG:293943.1:2000SEP08 | 6425161F7 | 254 | 537 |
| 33 | LG:293943.1:2000SEP08 | 7009531H1 | 384 | 720 |
| 34 | LG:345884.1:2000SEP08 | 7760811J1 | 54 | 260 |
| 34 | LG:345884.1:2000SEP08 | g4991320 | 187 | 645 |
| 34 | LG:345884.1:2000SEP08 | 4676488T6 | 209 | 615 |
| 34 | LG:345884.1:2000SEP08 | g2240240 | 1 | 310 |
| 34 | LG:345884.1:2000SEP08 | 4676488H1 | 2 | 258 |
| 34 | LG:345884.1:2000SEP08 | 4676488F6 | 1 | 387 |
| 35 | LG:400967.1:2000SEP08 | g2161571 | 1 | 424 |
| 35 | LG:400967.1:2000SEP08 | 184168H1 | 1 | 169 |
| 35 | LG:400967.1:2000SEP08 | 5491896H1 | 16 | 250 |
| 35 | LG:400967.1:2000SEP08 | 5772680H 1 | 36 | 528 |
| 35 | LG:400967.1:2000SEP08 | 3156449H1 | 183 | 343 |
| 35 | LG:400967.1:2000SEP08 | g3151140 | 215 | 672 |
| 35 | LG:400967.1:2000SEP08 | g2656954 | 333 | 629 |
| 35 | LG:400967.1:2000SEP08 | g5745679 | 375 | 664 |
| 35 | LG:400967.1:2000SEP08 | g2162512 | 511 | 664 |
| 36 | LG:024556.6:2000SEP08 | 5875448H1 | 1 | 284 |
| 36 | LG:024556.6:2000SEP08 | 5413554F6 | 4 | 491 |
| 36 | LG:024556.6:2000SEP08 | 6329517H1 | 6 | 146 |
| 36 | LG:024556.6:2000SEP08 | 7190073H2 | 34 | 527 |
| 36 | LG:024556.6:2000SEP08 | 3758768H1 | 37 | 307 |
| 36 | LG:024556.6:2000SEP08 | 5370118H1 | 54 | 301 |
| 36 | LG:024556.6:2000SEP08 | 537011 8F6 | 54 | 636 |
| 36 | LG:024556.6:2000SEP08 | g764662 | 96 | 484 |
| 36 | LG:024556.6:2000SEP08 | g874234 | 96 | 444 |
| 36 | LG:024556.6:2000SEP08 | g571259 | 103 | 459 |
| 36 | LG:024556.6:2000SEP08 | 5407536H1 | 239 | 489 |
| 36 | LG:024556.6:2000SEP08 | 7044534H1 | 250 | 451 |
| 36 | LG:024556.6:2000SEP08 | 7047004H 1 | 250 | 732 |
| 36 | LG:024556.6:2000SEP08 | 5423663F6 | 303 | 790 |
| 36 | LG:024556.6:2000SEP08 | 5423663H1 | 303 | 560 |
| 36 | LG:024556.6:2000SEP08 | g767562 | 343 | 633 |
| 36 | LG:024556.6:2000SEP08 | 5857590H1 | 401 | 647 |
| 36 | LG:024556.6:2000SEP08 | 6447737H1 | 469 | 654 |
| 37 | LG:081189.3:2000SEP08 | g4078107 | 458 | 904 |
| 37 | LG:081189.3:2000SEP08 | g5768539 | 453 | 904 |
| 37 | LG:081189.3:2000SEP08 | g4736702 | 453 | 905 |
| 37 | LG:081189.3:2000SEP08 | 6938064H1 | 465 | 871 |
| 37 | LG:081189.3:2000SEP08 | 6941504H1 | 468 | 853 |
| 37 | LG:081189.3:2000SEP08 | 2358206H1 | 336 | 414 |
| 37 | LG:081189.3:2000SEP08 | 1811707H1 | 225 | 465 |
| 37 | LG:081189.3:2000SEP08 | 1811707F6 | 225 | 698 |
| 37 | LG:081189.3:2000SEP08 | 7358905H1 | 1 | 478 |
| 38 | LG:018258.1:2000SEP08 | g1012122 | 1 | 131 |
| 38 | LG:018258.1:2000SEP08 | 1443432H1 | 1 | 253 |
| 38 | LG:018258.1:2000SEP08 | 1443432F6 | 1 | 268 |
| 38 | LG:018258.1:2000SEP08 | 1632787H1 | 140 | 373 |
| 38 | LG:018258.1:2000SEP08 | 1632787F6 | 140 | 610 |
| 38 | LG:018258.1:2000SEP08 | g1012076 | 184 | 489 |
| 38 | LG:018258.1:2000SEP08 | 5944405H1 | 374 | 533 |
| 39 | LG:450399.3:2000SEP08 | 5910662H1 | 1 | 295 |
| 39 | LG:450399.3:2000SEP08 | 5910662F6 | 1 | 552 |
| 39 | LG:450399.3:2000SEP08 | 5910662F8 | 1 | 510 |
| 39 | LG:450399.3:2000SEP08 | 5910662T6 | 9 | 605 |
| 39 | LG:450399.3:2000SEP08 | 5910662T9 | 67 | 576 |
| 39 | LG:450399.3:2000SEP08 | 5910662T8 | 146 | 593 |
| 40 | LG:451122.1:2000SEP08 | 6269521H1 | 5 | 418 |
| 40 | LG:451122.1:2000SEP08 | 5910718H1 | 1 | 288 |
| 40 | LG:451122.1:2000SEP08 | 5910718T9 | 280 | 348 |
| 40 | LG:451122.1:2000SEP08 | 5910718F8 | 1 | 457 |
| 40 | LG:451122.1:2000SEP08 | 5910718F6 | 1 | 448 |
| 40 | LG:451122.1:2000SEP08 | 5910718T6 | 1 | 422 |
| 40 | LG:451122.1:2000SEP08 | 6269521F8 | 5 | 448 |
| 40 | LG:451122.1:2000SEP08 | 5910718T8 | 1 | 361 |
| 41 | LG:451682.1:2000SEP08 | 5913661H1 | 1 | 283 |
| 41 | LG:451682.1:2000SEP08 | 5913661F8 | 1 | 569 |
| 41 | LG:451682.1:2000SEP08 | 5913661F6 | 1 | 575 |
| 41 | LG:451682.1:2000SEP08 | 5913661T6 | 213 | 821 |
| 42 | LG:238631.4:2000SEP08 | g1277788 | 1 | 426 |
| 42 | LG:238631.4:2000SEP08 | 5406438H1 | 1 | 261 |
| 42 | LG:238631.4:2000SEP08 | 5951609H1 | 3 | 368 |
| 42 | LG:238631.4:2000SEP08 | 586434H1 | 4 | 314 |
| 42 | LG:238631.4:2000SEP08 | 3120923H1 | 5 | 313 |
| 42 | LG:238631.4:2000SEP08 | 4815047H1 | 10 | 276 |
| 42 | LG:238631.4:2000SEP08 | 3743301H1 | 12 | 255 |
| 42 | LG:238631.4:2000SEP08 | 3736553H1 | 86 | 318 |
| 42 | LG:238631.4:2000SEP08 | 5598455H1 | 292 | 540 |
| 42 | LG:238631.4:2000SEP08 | g5855963 | 384 | 846 |
| 42 | LG:238631.4:2000SEP08 | g4070653 | 392 | 850 |
| 42 | LG:238631.4:2000SEP08 | g3665831 | 396 | 847 |
| 42 | LG:238631.4:2000SEP08 | g3246602 | 400 | 858 |
| 42 | LG:238631.4:2000SEP08 | g2161232 | 410 | 665 |
| 42 | LG:238631.4:2000SEP08 | g4525307 | 437 | 848 |
| 42 | LG:238631.4:2000SEP08 | g1404313 | 467 | 847 |
| 42 | LG:238631.4:2000SEP08 | g3253470 | 464 | 858 |
| 42 | LG:238631.4:2000SEP08 | g3250117 | 469 | 847 |
| 42 | LG:238631.4:2000SEP08 | g2269451 | 484 | 847 |
| 42 | LG:238631.4:2000SEP08 | g3735117 | 484 | 845 |
| 42 | LG:238631.4:2000SEP08 | g4223250 | 506 | 848 |
| 42 | LG:238631.4:2000SEP08 | g5766307 | 542 | 847 |
| 42 | LG:238631.4:2000SEP08 | g3737372 | 551 | 847 |
| 42 | LG:238631.4:2000SEP08 | g3016184 | 580 | 858 |
| 42 | LG:238631.4:2000SEP08 | g3734551 | 604 | 845 |
| 42 | LG:238631.4:2000SEP08 | g5447167 | 621 | 845 |
| 42 | LG:238631.4:2000SEP08 | g4762162 | 621 | 845 |
| 42 | LG:238631.4:2000SEP08 | g1231962 | 670 | 848 |
| 42 | LG:238631.4:2000SEP08 | 2406581H1 | 12 | 116 |
| 42 | LG:238631.4:2000SEP08 | 3401789H1 | 13 | 239 |
| 42 | LG:238631.4:2000SEP08 | 4648809H1 | 13 | 297 |
| 42 | LG:238631.4:2000SEP08 | 3634010H1 | 15 | 201 |
| 42 | LG:238631.4:2000SEP08 | 4653815H1 | 16 | 316 |
| 42 | LG:238631.4:2000SEP08 | 3152423H1 | 13 | 281 |
| 42 | LG:238631.4:2000SEP08 | 4414637H1 | 12 | 275 |
| 42 | LG:238631.4:2000SEP08 | 6121345H1 | 14 | 648 |
| 42 | LG:238631.4:2000SEP08 | 3428203H1 | 15 | 274 |
| 42 | LG:238631.4:2000SEP08 | 3744645H1 | 16 | 309 |
| 42 | LG:238631.4:2000SEP08 | 2698861H1 | 18 | 305 |
| 42 | LG:238631.4:2000SEP08 | 4143702H1 | 18 | 299 |
| 42 | LG:238631.4:2000SEP08 | 3995879H1 | 18 | 309 |
| 42 | LG:238631.4:2000SEP08 | 1830216H1 | 18 | 247 |
| 42 | LG:238631.4:2000SEP08 | 2998645H1 | 19 | 295 |
| 42 | LG:238631.4:2000SEP08 | 5464919H1 | 19 | 306 |
| 42 | LG:238631.4:2000SEP08 | 1713036H1 | 19 | 257 |
| 42 | LG:238631.4:2000SEP08 | 6116806H1 | 19 | 314 |
| 42 | LG:238631.4:2000SEP08 | 5788396H1 19 | 305 | |
| 42 | LG:238631.4:2000SEP08 | 5121572H1 | 19 | 299 |
| 42 | LG:238631.4:2000SEP08 | 4715287H1 | 20 | 256 |
| 42 | LG:238631.4:2000SEP08 | 6197104H1 | 23 | 561 |
| 42 | LG:238631.4:2000SEP08 | 493734H1 | 28 | 246 |
| 42 | LG:238631.4:2000SEP08 | 3988991H1 | 40 | 341 |
| 42 | LG:238631.4:2000SEP08 | 2048688H1 | 59 | 314 |
| 42 | LG:238631.4:2000SEP08 | 2757550H1 | 59 | 328 |
| 42 | LG:238631.4:2000SEP08 | 2755553H1 | 59 | 330 |
| 43 | LG:236654.1:2000SEP08 | 670448T6 | 802 | 1151 |
| 43 | LG:236654.1:2000SEP08 | 677184R6 | 802 | 1154 |
| 43 | LG:236654.1:2000SEP08 | g2810503 | 842 | 1188 |
| 43 | LG:236654.1:2000SEP08 | g2336018 | 909 | 1189 |
| 43 | LG:236654.1:2000SEP08 | 2892611H1 | 947 | 1180 |
| 43 | LG:236654.1:2000SEP08 | 1289991H1 | 1083 | 1189 |
| 43 | LG:236654.1:2000SEP08 | g1979821 | 1 | 187 |
| 43 | LG:236654.1:2000SEP08 | g3835204 | 1 | 325 |
| 43 | LG:236654.1:2000SEP08 | g6990824 | 1 | 298 |
| 43 | LG:236654.1:2000SEP08 | g6704413 | 1 | 299 |
| 43 | LG:236654.1:2000SEP08 | 6757618J1 | 8 | 93 |
| 43 | LG:236654.1:2000SEP08 | 7604986J1 | 9 | 544 |
| 43 | LG:236654.1:2000SEP08 | g4072180 | 18 | 469 |
| 43 | LG:236654.1:2000SEP08 | 3519808H1 | 80 | 368 |
| 43 | LG:236654.1:2000SEP08 | g843784 | 234 | 500 |
| 43 | LG:236654.1:2000SEP08 | 1614746F6 | 288 | 734 |
| 43 | LG:236654.1:2000SEP08 | 1614746H1 | 288 | 506 |
| 43 | LG:236654.1:2000SEP08 | 7604986H1 | 364 | 845 |
| 43 | LG:236654.1:2000SEP08 | 1575232H1 | 366 | 578 |
| 43 | LG:236654.1:2000SEP08 | g2209653 | 404 | 914 |
| 43 | LG:236654.1:2000SEP08 | 6322462H1 | 521 | 720 |
| 43 | LG:236654.1:2000SEP08 | 2892611T6 | 647 | 1138 |
| 43 | LG:236654.1:2000SEP08 | g5660108 | 723 | 1188 |
| 43 | LG:236654.1:2000SEP08 | 1614746T6 | 735 | 1150 |
| 43 | LG:236654.1:2000SEP08 | g5365441 | 749 | 1190 |
| 43 | LG:236654.1:2000SEP08 | g3644249 | 768 | 1191 |
| 43 | LG:236654.1:2000SEP08 | 677184H1 | 802 | 1073 |
| 43 | LG:236654.1:2000SEP08 | 670448R6 | 802 | 1188 |
| 43 | LG:236654.1:2000SEP08 | 670448H1 | 802 | 1057 |
| 44 | LG:332655.1:2000SEP08 | 570522H1 | 606 | 876 |
| 44 | LG:332655.1:2000SEP08 | 3872248T6 | 663 | 1142 |
| 44 | LG:332655.1:2000SEP08 | 2850393T6 | 700 | 1142 |
| 44 | LG:332655.1:2000SEP08 | g2835010 | 716 | 1069 |
| 44 | LG:332655.1:2000SEP08 | g4763947 | 847 | 1144 |
| 44 | LG:332655.1:2000SEP08 | g4986367 | 978 | 1144 |
| 44 | LG:332655.1:2000SEP08 | g5661482 | 980 | 1142 |
| 44 | LG:332655.1:2000SEP08 | 516193H1 | 1027 | 1136 |
| 44 | LG:332655.1:2000SEP08 | 5070935H1 | 498 | 757 |
| 44 | LG:332655.1:2000SEP08 | g1959326 | 479 | 914 |
| 44 | LG:332655.1:2000SEP08 | g1947735 | 206 | 551 |
| 44 | LG:332655.1:2000SEP08 | 3096881H1 | 265 | 401 |
| 44 | LG:332655.1:2000SEP08 | 3098886H1 | 275 | 541 |
| 44 | LG:332655.1:2000SEP08 7600091H1 | 314 | 782 | |
| 44 | LG:332655.1:2000SEP08 | 2850393F6 | 1 499 | |
| 44 | LG:332655.1:2000SEP08 | 2850393H1 | 2 | 289 |
| 44 | LG:332655.1:2000SEP08 | 861289H1 | 3 | 241 |
| 44 | LG:332655.1:2000SEP08 | 2287640R6 | 5 | 485 |
| 44 | LG:332655.1:2000SEP08 | 2287640H1 | 5 | 255 |
| 44 | LG:332655.1:2000SEP08 | 3872248F6 | 11 | 517 |
| 44 | LG:332655.1:2000SEP08 | 3872248H1 | 11 | 301 |
| 44 | LG:332655.1:2000SEP08 | 3403385H1 | 114 | 366 |
| 44 | LG:332655.1:2000SEP08 | 1821848H1 | 133 | 370 |
| 44 | LG:332655.1:2000SEP08 | 3531915H1 | 153 | 445 |
| 44 | LG:332655.1:2000SEP08 | 2059050H1 | 160 | 405 |
| 44 | LG:332655.1:2000SEP08 | 5742858H1 | 164 | 474 |
| 44 | LG:332655.1:2000SEP08 | 2733553H1 | 185 | 415 |
| 44 | LG:332655.1:2000SEP08 | 5025820H1 | 188 | 470 |
| 45 | LG:217396.2:2000SEP08 | 5628336R6 | 940 | 1483 |
| 45 | LG:217396.2:2000SEP08 | 6265192H1 | 695 | 1273 |
| 45 | LG:217396.2:2000SEP08 | 5868595H1 | 984 | 1252 |
| 45 | LG:217396.2:2000SEP08 | 5868595F6 | 972 | 1252 |
| 45 | LG:217396.2:2000SEP08 | g1187240 | 976 | 1186 |
| 45 | LG:217396.2:2000SEP08 | 7676247J2 | 625 | 1182 |
| 45 | LG:217396.2:2000SEP08 | 7706403H1 | 548 | 1138 |
| 45 | LG:217396.2:2000SEP08 | g1195854 | 640 | 960 |
| 45 | LG:217396.2:2000SEP08 | 7351185H1 | 427 | 957 |
| 45 | LG:217396.2:2000SEP08 | g4648085 | 606 | 876 |
| 45 | LG:217396.2:2000SEP08 | 5071261H1 | 578 | 837 |
| 45 | LG:217396.2:2000SEP08 | 5071261F6 | 578 | 824 |
| 45 | LG:217396.2:2000SEP08 | g1921151 | 518 | 739 |
| 45 | LG:217396.2:2000SEP08 | 6875193H1 | 159 | 671 |
| 45 | LG:21 7396.2:2000SEP08 | 6989406H1 | 250 | 649 |
| 45 | LG:217396.2:2000SEP08 | g1921152 | 105 | 513 |
| 45 | LG:217396.2:2000SEP08 | 2996140F6 | 1 | 621 |
| 45 | LG:217396.2:2000SEP08 | g2818339 | 123 | 530 |
| 45 | LG:217396.2:2000SEP08 | 3750696H1 | 232 | 517 |
| 45 | LG:217396.2:2000SEP08 | 7676247H2 | 1 | 401 |
| 45 | LG:217396.2:2000SEP08 | 5071261T6 | 1 | 375 |
| 45 | LG:217396.2:2000SEP08 | 2996140H1 | 1 | 265 |
| 46 | LG:090574.1:2000SEP08 | 2659966F6 | 1 | 192 |
| 46 | LG:090574.1:2000SEP08 | 2659966T6 | 1 | 194 |
| 46 | LG:090574.1:2000SEP08 | 2659966H1 | 1 | 247 |
| 47 | LG:202943.1:2000SEP08 | 1226590R6 | 174 | 583 |
| 47 | LG:202943.1:2000SEP08 | 5639884H1 | 136 | 365 |
| 47 | LG:202943.1:2000SEP08 | 1226590T6 | 383 | 888 |
| 47 | LG:202943.1:2000SEP08 | 5092132H1 | 515 | 763 |
| 47 | LG:202943.1:2000SEP08 | 1226590H1 | 174 | 418 |
| 47 | LG:202943.1:2000SEP08 | g2020858 | 180 | 420 |
| 47 | LG:202943.1:2000SEP08 | 7213845H1 | 242 | 792 |
| 47 | LG:202943.1:2000SEP08 | 2579002H2 | 975 | 1177 |
| 47 | LG:202943.1:2000SEP08 | 6472564H1 | 942 | 1357 |
| 47 | LG:202943.1:2000SEP08 | 7151349H1 | 58 | 561 |
| 47 | LG:202943.1:2000SEP08 | 5089408H1 | 1 | 270 |
| 47 | LG:202943.1:2000SEP08 | 5091008H1 | 1 | 269 |
| 47 | LG:202943.1:2000SEP08 | 7594783H1 | 818 | 1226 |
| 47 | LG:202943.1:2000SEP08 | 6469878H1 | 624 | 1185 |
| 47 | LG:202943.1:2000SEP08 | 5092608H1 | 1 | 274 |
| 48 | LG:236928.1:2000SEP08 | 3227840H1 | 1509 | 1789 |
| 48 | LG:236928.1:2000SEP08 | 2487371H1 | 1524 | 1754 |
| 48 | LG:236928.1:2000SEP08 | 2908866H1 | 1579 | 1768 |
| 48 | LG:236928.1:2000SEP08 | g2538920 | 1584 | 1877 |
| 48 | LG:236928.1:2000SEP08 | 487800H1 | 1633 | 1905 |
| 48 | LG:236928.1:2000SEP08 | 5404331H1 | 1669 | 1966 |
| 48 | LG:236928.1:2000SEP08 | 2904955T6 | 1672 | 2110 |
| 48 | LG:236928.1:2000SEP08 | 4243773H1 | 1679 | 1938 |
| 48 | LG:236928.1:2000SEP08 | 2904624H1 | 1682 | 1978 |
| 48 | LG:236928.1:2000SEP08 | g4990382 | 1689 | 2149 |
| 48 | LG:236928.1:2000SEP08 | 3291485T6 | 1692 | 2110 |
| 48 | LG:236928.1:2000SEP08 | 7365257H1 | 1694 | 2145 |
| 48 | LG:236928.1:2000SEP08 | g3144565 | 1717 | 2158 |
| 48 | LG:236928.1:2000SEP08 | g3144841 | 1723 | 2149 |
| 48 | LG:236928.1:2000SEP08 | 7761153H1 | 1 | 630 |
| 48 | LG:236928.1:2000SEP08 | 2905587H1 | 26 | 296 |
| 48 | LG:236928.1:2000SEP08 | 7741081H1 | 173 | 752 |
| 48 | LG:236928.1:2000SEP08 | 1491957H1 | 180 | 280 |
| 48 | LG:236928.1:2000SEP08 | 4741520H1 | 195 | 447 |
| 48 | LG:236928.1:2000SEP08 | 4741520F6 | 195 | 579 |
| 48 | LG:236928.1:2000SEP08 | 6616428H1 | 203 | 717 |
| 48 | LG:236928.1:2000SEP08 | 4697183H1 | 235 | 483 |
| 48 | LG:236928.1:2000SEP08 | 4697183F6 | 235 | 679 |
| 48 | LG:236928.1:2000SEP08 | 4028773H1 | 432 | 694 |
| 48 | LG:236928.1:2000SEP08 | 4028773F8 | 441 | 957 |
| 48 | LG:236928.1:2000SEP08 | 7761153J1 | 472 | 1051 |
| 48 | LG:236928.1:2000SEP08 | 7109534H1 524 | 1104 | |
| 48 | LG:236928.1:2000SEP08 | 3291 485F6 | 798 | 1256 |
| 48 | LG:236928.1:2000SEP08 | 2285984H1 | 798 | 1018 |
| 48 | LG:236928.1:2000SEP08 | 3291485H1 | 800 | 1044 |
| 48 | LG:236928.1:2000SEP08 | 6842952H1 | 822 | 1148 |
| 48 | LG:236928.1:2000SEP08 | 6018945H1 | 841 | 1148 |
| 48 | LG:236928.1:2000SEP08 | 6616428J1 | 847 | 1412 |
| 48 | LG:236928.1:2000SEP08 | 4785174H1 | 867 | 1127 |
| 48 | LG:236928.1:2000SEP08 | 2904955F6 | 1086 | 1487 |
| 48 | LG:236928.1:2000SEP08 | 2904955H1 | 1086 | 1380 |
| 48 | LG:236928.1:2000SEP08 | g3163646 | 1101 | 1348 |
| 48 | LG:236928.1:2000SEP08 | 2918045H1 | 1170 | 1452 |
| 48 | LG:236928.1:2000SEP08 | 4740283H1 | 1210 | 1470 |
| 48 | LG:236928.1:2000SEP08 | g1962038 | 1291 | 1631 |
| 48 | LG:236928.1:2000SEP08 | 1251534H1 | 1448 | 1674 |
| 48 | LG:236928.1:2000SEP08 | 1251534F6 | 1448 | 1853 |
| 48 | LG:236928.1:2000SEP08 | 2556286H1 | 1483 | 1733 |
| 48 | LG:236928.1:2000SEP08 | 6131688H1 | 1790 | 2071 |
| 48 | LG:236928.1:2000SEP08 | g3679201 | 1791 | 2159 |
| 48 | LG:236928.1:2000SEP08 | 2919136H1 | 1807 | 2066 |
| 48 | LG:236928.1:2000SEP08 | 6442673H1 | 1814 | 2356 |
| 48 | LG:236928.1:2000SEP08 | 6870682H1 | 1869 | 2347 |
| 48 | LG:236928.1:2000SEP08 | 6438933H1 | 1871 | 2347 |
| 48 | LG:236928.1:2000SEP08 | 645255H1 | 1877 | 2114 |
| 48 | LG:236928.1:2000SEP08 | 1251534T7 | 1922 | 2108 |
| 48 | LG:236928.1:2000SEP08 | 2288452H1 | 1966 | 2122 |
| 48 | LG:236928.1:2000SEP08 | 6855869H1 | 1968 | 2537 |
| 48 | LG:236928.1:2000SEP08 | 1296086F6 | 2002 | 2338 |
| 48 | LG:236928.1:2000SEP08 | 1296086H1 | 2002 | 2237 |
| 48 | LG:236928.1:2000SEP08 | 5021561H1 | 2022 | 2302 |
| 48 | LG:236928.1:2000SEP08 | 6616156H1 | 2040 | 2488 |
| 48 | LG:236928.1:2000SEP08 | g1624634 | 2135 | 2305 |
| 48 | LG:236928.1:2000SEP08 | 3638554H1 | 2167 | 2353 |
| 48 | LG:236928.1:2000SEP08 | 3356574H1 | 2207 | 2428 |
| 48 | LG:236928.1:2000SEP08 | 531771R6 | 2245 | 2582 |
| 48 | LG:236928.1:2000SEP08 | 531771H1 | 2245 | 2486 |
| 48 | LG:236928.1:2000SEP08 | 2556128F6 | 2315 | 2567 |
| 48 | LG:236928.1:2000SEP08 | 2556128H1 | 2315 | 2566 |
| 48 | LG:236928.1:2000SEP08 | g1186813 | 2339 | 2535 |
| 48 | LG:236928.1:2000SEP08 | 2781958H1 | 2345 | 2603 |
| 48 | LG:236928.1:2000SEP08 | 4748766H1 | 2371 | 2635 |
| 48 | LG:236928.1:2000SEP08 | 4748737H1 | 2371 | 2635 |
| 48 | LG:236928.1:2000SEP08 | 3563133H1 | 2372 | 2566 |
| 48 | LG:236928.1:2000SEP08 | 531771T6 | 2411 | 2935 |
| 48 | LG:236928.1:2000SEP08 | 5021561T1 | 2492 | 2929 |
| 48 | LG:236928.1:2000SEP08 | 1269755F6 | 2505 | 2772 |
| 48 | LG:236928.1:2000SEP08 | 1269755H1 | 2505 | 2761 |
| 48 | LG:236928.1:2000SEP08 | 1269755F1 | 2505 | 2873 |
| 48 | LG:236928.1:2000SEP08 | 1269755T6 | 2506 | 2934 |
| 48 | LG:236928.1:2000SEP08 | 556128T6 | 2511 | 2933 |
| 48 | LG:236928.1:2000SEP08 | g2106737 | 2522 | 2954 |
| 48 | LG:236928.1:2000SEP08 | 6138111H1 | 2539 | 2849 |
| 48 | LG:236928.1:2000SEP08 | g3179767 | 2580 | 2982 |
| 48 | LG:236928.1:2000SEP08 | 727813H1 | 2595 | 2826 |
| 48 | LG:236928.1:2000SEP08 | 4367390H2 | 2598 | 2853 |
| 48 | LG:236928.1:2000SEP08 | g1190057 | 2621 | 2982 |
| 48 | LG:236928.1:2000SEP08 | g4986351 | 2683 | 2973 |
| 48 | LG:236928.1:2000SEP08 | 92526245 | 2756 | 2973 |
| 48 | LG:236928.1:2000SEP08 | 1717327T6 | 2855 | 3383 |
| 48 | LG:236928.1:2000SEP08 | 1717327H1 | 2862 | 3109 |
| 48 | LG:236928.1:2000SEP08 | 1717327F6 | 2862 | 3351 |
| 49 | LG:215169.2:2000SEP08 | 6037163H1 | 602 | 1129 |
| 49 | LG:215169.2:2000SEP08 | 6207071H1 | 928 | 1536 |
| 49 | LG:215169.2:2000SEP08 | 7098455H1 | 1246 | 1637 |
| 49 | LG:215169.2:2000SEP08 | 6970186H1 | 1305 | 1880 |
| 49 | LG:215169.2:2000SEP08 | g5176731 | 1 | 212 |
| 49 | LG:215169.2:2000SEP08 | g5431398 | 1 | 377 |
| 49 | LG:215169.2:2000SEP08 | g4114328 | 1 | 456 |
| 49 | LG:215169.2:2000SEP08 | g6704290 | 1 | 472 |
| 49 | LG:215169.2:2000SEP08 | 6477953H1 | 1373 | 1881 |
| 49 | LG:215169.2:2000SEP08 | 7714805H1 | 1384 | 1978 |
| 49 | LG:215169.2:2000SEP08 | 1486266H1 | 1519 | 1767 |
| 49 | LG:215169.2:2000SEP08 | 2843912H1 | 1718 | 1991 |
| 49 | LG:215169.2:2000SEP08 | 1384143H1 | 1883 | 2133 |
| 49 | LG:215169.2:2000SEP08 | 4901661H1 | 436 | 630 |
| 49 | LG:215169.2:2000SEP08 | 4901661F6 | 436 | 633 |
| 49 | LG:215169.2:2000SEP08 | 7269396H1 | 112 | 652 |
| 49 | LG:215169.2:2000SEP08 | 7322496H1 | 156 | 636 |
| 49 | LG:215169.2:2000SEP08 | 6314811H1 | 311 | 833 |
| 49 | LG:215169.2:2000SEP08 | g5664682 | 18 | 244 |
| 50 | LG:410726.1:2000SEP08 | 2071327H1 | 360 | 550 |
| 50 | LG:410726.1:2000SEP08 | 6912958J1 | 426 | 1019 |
| 50 | LG:410726.1:2000SEP08 | 4583753H1 | 453 | 741 |
| 50 | LG:410726.1:2000SEP08 | 7699696J1 | 724 | 1108 |
| 50 | LG:410726.1:2000SEP08 | 207132716 | 751 | 1186 |
| 50 | LG:410726.1:2000SEP08 | g4176194 | 788 | 1231 |
| 50 | LG:410726.1:2000SEP08 | g3178308 | 806 | 1226 |
| 50 | LG:410726.1:2000SEP08 | g4149782 | 899 | 1217 |
| 50 | LG:410726.1:2000SEP08 | 6912958H1 | 912 | 1382 |
| 50 | LG:410726.1:2000SEP08 | g3797002 | 1049 | 1163 |
| 50 | LG:410726.1:2000SEP08 | g3180193 | 1210 | 1289 |
| 50 | LG:410726.1:2000SEP08 | 6534376H1 | 1 | 277 |
| 50 | LG:410726.1:2000SEP08 | 6392976H1 | 1 | 276 |
| 50 | LG:410726.1:2000SEP08 | 7233013H1 | 68 | 590 |
| 50 | LG:410726.1:2000SEP08 | 6033171F6 | 69 | 623 |
| 50 | LG:410726.1:2000SEP08 | 6033171H1 | 74 | 509 |
| 50 | LG:410726.1:2000SEP08 | g1648352 | 98 | 503 |
| 50 | LG:410726.1:2000SEP08 | 3023715H1 | 259 | 523 |
| 50 | LG:410726.1:2000SEP08 | 2071327F6 | 360 | 659 |
| 51 | LG:234372.2:2000SEP08 | 1661362T6 | 2456 | 2794 |
| 51 | LG:234372.2:2000SEP08 | g2277847 | 2649 | 2848 |
| 51 | LG:234372.2:2000SEP08 | g3423969 | 2480 | 2842 |
| 51 | LG:234372.2:2000SEP08 | g2216700 | 2476 | 2633 |
| 51 | LG:234372.2:2000SEP08 | 1550682R6 | 2348 | 2616 |
| 51 | LG:234372.2:2000SEP08 | g4243239 | 2398 | 2838 |
| 51 | LG:234372.2:2000SEP08 | 5487834H1 | 2403 | 2668 |
| 51 | LG:234372.2:2000SEP08 | 2097573H1 | 2418 | 2567 |
| 51 | LG:234372.2:2000SEP08 | g4535323 | 2421 | 2633 |
| 51 | LG:234372.2:2000SEP08 | g3840702 | 2425 | 2837 |
| 51 | LG:234372.2:2000SEP08 | 266654216 | 2424 | 2800 |
| 51 | LG:234372.2:2000SEP08 | 1537770H1 | 2437 | 2657 |
| 51 | LG:234372.2:2000SEP08 | 1796389H1 | 2443 | 2647 |
| 51 | LG:234372.2:2000SEP08 | 5065143H1 | 1951 | 2181 |
| 51 | LG:234372.2:2000SEP08 | 3692831H1 | 1973 | 2248 |
| 51 | LG:234372.2:2000SEP08 | 2436249H1 | 1989 | 2183 |
| 51 | LG:234372.2:2000SEP08 | 3552362H1 | 1989 | 2135 |
| 51 | LG:234372.2:2000SEP08 | 907632R2 | 2042 | 2608 |
| 51 | LG:234372.2:2000SEP08 | 5024569H1 | 2057 | 2324 |
| 51 | LG:234372.2:2000SEP08 | 35250769H1 | 2057 | 2298 |
| 51 | LG:234372.2:2000SEP08 | 2689102H1 | 2087 | 2341 |
| 51 | LG:234372.2:2000SEP08 | 3601859H1 | 2094 | 2400 |
| 51 | LG:234372.2:2000SEP08 | 980210H1 | 2099 | 2420 |
| 51 | LG:234372.2:2000SEP08 | 2683701H1 | 2101 | 2409 |
| 51 | LG:234372.2:2000SEP08 | 1661356H1 | 2129 | 2344 |
| 51 | LG:234372.2:2000SEP08 | 1661362H1 | 2129 | 2338 |
| 51 | LG:234372.2:2000SEP08 | 1661362F6 | 2129 | 2473 |
| 51 | LG:234372.2:2000SEP08 | 7751770J1 | 2137 | 2800 |
| 51 | LG:234372.2:2000SEP08 | 184042416 | 2178 | 2800 |
| 51 | LG:234372.2:2000SEP08 | 220074H1 | 2211 | 2385 |
| 51 | LG:234372.2:2000SEP08 | 7348566H1 | 2237 | 2837 |
| 51 | LG:234372.2:2000SEP08 | 6543746H1 | 2239 | 2820 |
| 51 | LG:234372.2:2000SEP08 | 261600316 | 2249 | 2797 |
| 51 | LG:234372.2:2000SEP08 | 6846460H1 | 2275 | 2812 |
| 51 | LG:234372.2:2000SEP08 | 401177616 | 2286 | 2800 |
| 51 | LG:234372.2:2000SEP08 | 7329765H1 | 2302 | 2789 |
| 51 | LG:234372.2:2000SEP08 | 590936F1 | 2314 | 2837 |
| 51 | LG:234372.2:2000SEP08 | 1511731T6 | 2317 | 2793 |
| 51 | LG:234372.2:2000SEP08 | 5103857H1 | 2345 | 2565 |
| 51 | LG:234372.2:2000SEP08 | 155068216 | 2348 | 2787 |
| 51 | LG:234372.2:2000SEP08 | 1550682H1 | 2348 | 2558 |
| 51 | LG:234372.2:2000SEP08 | g1781803 | 1370 | 1603 |
| 51 | LG:234372.2:2000SEP08 | g883240 | 1432 | 1757 |
| 51 | LG:234372.2:2000SEP08 | 971981R6 | 1447 | 1892 |
| 51 | LG:234372.2:2000SEP08 | 971981H1 | 1447 | 1734 |
| 51 | LG:234372.2:2000SEP08 | 600414H1 | 1450 | 1654 |
| 51 | LG:234372.2:2000SEP08 | 4640503H1 | 1463 | 1733 |
| 51 | LG:234372.2:2000SEP08 | 4096777H1 | 1476 | 1778 |
| 51 | LG:234372.2:2000SEP08 | 7751770H1 | 1485 | 1986 |
| 51 | LG:234372.2:2000SEP08 | 5799040H1 | 1483 | 1965 |
| 51 | LG:234372.2:2000SEP08 | 4084077H1 | 1511 | 1780 |
| 51 | LG:234372.2:2000SEP08 | 4650278H1 | 1546 | 1821 |
| 51 | LG:234372.2:2000SEP08 | 2541227H1 | 1552 | 1796 |
| 51 | LG:234372.2:2000SEP08 | 7653281H1 | 1556 | 2161 |
| 51 | LG:234372.2:2000SEP08 | 2308666H1 | 1612 | 1801 |
| 51 | LG:234372.2:2000SEP08 | 1719968F6 | 1614 | 2114 |
| 51 | LG:234372.2:2000SEP08 | 1719968H1 | 1614 | 1837 |
| 51 | LG:234372.2:2000SEP08 | 7322428H1 | 1727 | 2305 |
| 51 | LG:234372.2:2000SEP08 | g1646882 | 1750 | 2182 |
| 51 | LG:234372.2:2000SEP08 | 4317152H1 | 1763 | 2035 |
| 51 | LG:234372.2:2000SEP08 | 5301059F8 | 1776 | 2249 |
| 51 | LG:234372.2:2000SEP08 | 7940028H1 | 1829 | 2452 |
| 51 | LG:234372.2:2000SEP08 | 2666542F6 | 1854 | 2343 |
| 51 | LG:234372.2:2000SEP08 | 2666542H1 | 1854 | 2099 |
| 51 | LG:234372.2:2000SEP08 | 964975H1 | 1879 | 1991 |
| 51 | LG:234372.2:2000SEP08 | 1381321H1 | 1890 | 2142 |
| 51 | LG:234372.2:2000SEP08 | 5979524H1 | 1891 | 2177 |
| 51 | LG:234372.2:2000SEP08 | 7060659H1 | 1895 | 2448 |
| 51 | LG:234372.2:2000SEP08 | 2616003H1 | 799 | 1062 |
| 51 | LG:234372.2:2000SEP08 | 2616003F6 | 799 | 1287 |
| 51 | LG:234372.2:2000SEP08 | 7406896H1 | 837 | 1173 |
| 51 | LG:234372.2:2000SEP08 | 3086412H1 | 861 | 1155 |
| 51 | LG:234372.2:2000SEP08 | 6075203H1 | 879 | 1174 |
| 51 | LG:234372.2:2000SEP08 | 7653281J1 | 888 | 1467 |
| 51 | LG:234372.2:2000SEP08 | 5059222H1 | 1071 | 1336 |
| 51 | LG:234372.2:2000SEP08 | 590936H1 | 1088 | 1346 |
| 51 | LG:234372.2:2000SEP08 | 590970H1 | 1088 | 1209 |
| 51 | LG:234372.2:2000SEP08 | 590936R1 | 1088 | 1612 |
| 51 | LG:234372.2:2000SEP08 | 1317631H1 | 1091 | 1427 |
| 51 | LG:234372.2:2000SEP08 | 3601816H1 | 1109 | 1413 |
| 51 | LG:234372.2:2000SEP08 | 3529032H1 | 1114 | 1423 |
| 51 | LG:234372.2:2000SEP08 | 3116380H1 | 1123 | 1407 |
| 51 | LG:234372.2:2000SEP08 | 3117081H1 | 1124 | 1391 |
| 51 | LG:234372.2:2000SEP08 | 4405926H1 | 1137 | 1397 |
| 51 | LG:234372.2:2000SEP08 | 1511731F6 | 1143 | 1655 |
| 51 | LG:234372.2:2000SEP08 | 2841541H1 | 1144 | 1399 |
| 51 | LG:234372.2:2000SEP08 | 1511731H1 | 1143 | 1328 |
| 51 | LG:234372.2:2000SEP08 | 1511356H1 | 1143 | 1327 |
| 51 | LG:234372.2:2000SEP08 | 4056493H1 | 1302 | 1580 |
| 51 | LG:234372.2:2000SEP08 | g1959546 | 1304 | 1747 |
| 51 | LG:234372.2:2000SEP08 | g1349612 | 1328 | 1793 |
| 51 | LG:234372.2:2000SEP08 | g880451 | 1336 | 1792 |
| 51 | LG:234372.2:2000SEP08 | g830969 | 1336 | 1711 |
| 51 | LG:234372.2:2000SEP08 | 2181970H1 | 1336 | 1606 |
| 51 | LG:234372.2:2000SEP08 | 5326001H2 | 1356 | 1608 |
| 51 | LG:234372.2:2000SEP08 | 5325701H1 | 1356 | 1610 |
| 51 | LG:234372.2:2000SEP08 | 1564961H1 | 1368 | 1557 |
| 51 | LG:234372.2:2000SEP08 | g2968054 | 2661 | 2838 |
| 51 | LG:234372.2:2000SEP08 | 7033565H1 | 1 | 576 |
| 51 | LG:234372.2:2000SEP08 | 4252317H1 | 46 | 191 |
| 51 | LG:234372.2:2000SEP08 | 7171693H1 | 221 | 736 |
| 51 | LG:234372.2:2000SEP08 | 7179847H1 | 221 | 651 |
| 51 | LG:234372.2:2000SEP08 | 140734H1 | 240 | 341 |
| 51 | LG:234372.2:2000SEP08 | 4938818H1 | 286 | 553 |
| 51 | LG:234372.2:2000SEP08 | 7592182H1 | 465 | 1020 |
| 51 | LG:234372.2:2000SEP08 | 1840424F6 | 478 | 838 |
| 51 | LG:234372.2:2000SEP08 | 1840424H1 | 478 | 755 |
| 51 | LG:234372.2:2000SEP08 | 4011776F6 | 494 | 963 |
| 51 | LG:234372.2:2000SEP08 | 4011776H1 | 496 | 749 |
| 51 | LG:234372.2:2000SEP08 | 2770053H1 | 556 | 809 |
| 51 | LG:234372.2:2000SEP08 | 7759056H1 | 616 | 937 |
| 51 | LG:234372.2:2000SEP08 | 4447071H1 | 636 | 912 |
| 51 | LG:234372.2:2000SEP08 | 5373391H1 | 767 | 999 |
| 51 | LG:234372.2:2000SEP08 | 1943237H1 | 2481 | 2735 |
| 51 | LG:234372.2:2000SEP08 | g2077756 | 2484 | 2840 |
| 51 | LG:234372.2:2000SEP08 | g883241 | 2495 | 2847 |
| 51 | LG:234372.2:2000SEP08 | g831477 | 2497 | 2849 |
| 51 | LG:234372.2:2000SEP08 | 5301059T9 | 2527 | 2732 |
| 51 | LG:234372.2:2000SEP08 | g3700868 | 2531 | 2837 |
| 51 | LG:234372.2:2000SEP08 | g3872992 | 2532 | 2842 |
| 51 | LG:234372.2:2000SEP08 | 971981T6 | 2531 | 2813 |
| 51 | LG:234372.2:2000SEP08 | 5102936H1 | 2539 | 2791 |
| 51 | LG:234372.2:2000SEP08 | g1664296 | 2539 | 2633 |
| 51 | LG:234372.2:2000SEP08 | 1292736H1 | 2564 | 2796 |
| 51 | LG:234372.2:2000SEP08 | 3112301H1 | 2565 | 2851 |
| 51 | LG:234372.2:2000SEP08 | g4109330 | 2565 | 2633 |
| 51 | LG:234372.2:2000SEP08 | g2968055 | 2573 | 2838 |
| 51 | LG:234372.2:2000SEP08 | g49713A4 | 2598 | 2839 |
| 51 | LG:234372.2:2000SEP08 | 2717958H1 | 2609 | 2837 |
| 51 | LG:234372.2:2000SEP08 | 2766115H1 | 2752 | 2837 |
| 52 | LG:022629.1:2000SEP08 | 3627874H1 | 253 | 538 |
| 52 | LG:022629.1:2000SEP08 | 5786985H1 | 209 | 495 |
| 52 | LG:022629.1:2000SEP08 | 3627896H1 | 242 | 449 |
| 52 | LG:022629.1:2000SEP08 | 5403369H1 | 1 | 231 |
| 52 | LG:022629.1:2000SEP08 | 540336919 | 337 | 738 |
| 52 | LG:022629.1:2000SEP08 | 6082254H1 | 201 | 724 |
| 52 | LG:022629.1:2000SEP08 | g2198283 | 364 | 668 |
| 52 | LG:022629.1:2000SEP08 | 5403369T8 | 192 | 649 |
| 52 | LG:022629.1:2000SEP08 | g3162701 | 219 | 638 |
| 52 | LG:022629.1:2000SEP08 | 3627896F6 | 155 | 599 |
| 52 | LG:022629.1:2000SEP08 | 5403369F8 | 19 | 594 |
| 52 | LG:022629.1:2000SEP08 | 1519408H1 | 354 | 551 |
| 52 | LG:022629.1:2000SEP08 | 1519416H1 | 354 | 541 |
| 52 | LG:022629.1:2000SEP08 | 7683101H1 | 748 | 1276 |
| 52 | LG:022629.1:2000SEP08 | 6569535H1 | 649 | 1171 |
| 52 | LG:022629.1:2000SEP08 | 1519416T6 | 592 | 877 |
| 52 | LG:022629.1:2000SEP08 | 5958910H1 | 759 | 865 |
| 52 | LG:022629.1:2000SEP08 | g6450919 | 360 | 843 |
| 52 | LG:022629.1:2000SEP08 | g6836892 | 618 | 843 |
| 52 | LG:022629.1:2000SEP08 | g4738617 | 377 | 842 |
| 52 | LG:022629.1:2000SEP08 | g4186678 | 404 | 842 |
| 52 | LG:022629.1:2000SEP08 | g5541594 | 602 | 840 |
| 52 | LG:022629.1:2000SEP08 | 3627896T6 | 288 | 800 |
| 52 | LG:022629.1:2000SEP08 | 1519416F6 | 354 | 771 |
| 52 | LG:022629.1:2000SEP08 | g2783063 | 400 | 762 |
| 52 | LG:022629.1:2000SEP08 | g2958447 | 367 | 762 |
| 52 | LG:022629.1:2000SEP08 | g2198251 | 454 | 762 |
| 52 | LG:022629.1:2000SEP08 | g2876399 | 393 | 762 |
| 53 | LG:068682.1:2000SEP08 2011384H1 | 190 | 263 | |
| 53 | LG:068682.1:2000SEP08 | g5438746 | 1 | 423 |
| 53 | LG:068682.1:2000SEP08 | g3805312 | 34 | 423 |
| 53 | LG:068682.1:2000SEP08 | g4372490 | 78 | 423 |
| 53 | LG:068682.1:2000SEP08 | g2954208 | 76 | 423 |
| 53 | LG:068682.1:2000SEP08 | g2954218 | 142 | 422 |
| 53 | LG:068682.1:2000SEP08 | g6838215 | 124 | 383 |
| 53 | LG:068682.1:2000SEP08 | g3307490 | 142 | 344 |
| 53 | LG:068682.1:2000SEP08 6829315W | 314 | 884 | |
| 53 | LG:068682.1:2000SEP08 | g3109791 | 492 | 811 |
| 53 | LG:068682.1:2000SEP08 | g5452473 | 492 | 650 |
| 53 | LG:068682.1:2000SEP08 | g4564783 | 26 | 423 |
| 53 | LG:068682.1:2000SEP08 | g6043518 | 78 | 423 |
| 54 | LG:222335.1:2000SEP08 | 6804715H1 | 612 | 1189 |
| 54 | LG:222335.1:2000SEP08 | 2317449H1 | 764 | 853 |
| 54 | LG:222335.1:2000SEP08 | 2583954F6 | 816 | 1189 |
| 54 | LG:222335.1:2000SEP08 | 2583954H1 | 816 | 1087 |
| 54 | LG:222335.1:2000SEP08 | 5591720H1 | 954 | 1099 |
| 54 | LG:222335.1:2000SEP08 | 785266H1 | 1056 | 1206 |
| 54 | LG:222335.1:2000SEP08 | 785266R6 | 1056 | 1423 |
| 54 | LG:222335.1:2000SEP08 | 6336926H1 | 1134 | 1419 |
| 54 | LG:222335.1:2000SEP08 | 6336926F8 | 1134 | 1420 |
| 54 | LG:222335.1:2000SEP08 | 7063506H1 | 1 | 370 |
| 54 | LG:222335.1:2000SEP08 | 3778951H1 | 1 | 282 |
| 54 | LG:222335.1:2000SEP08 | 6258260H1 | 1 | 284 |
| 54 | LG:222335.1:2000SEP08 | 5560479H1 | 7 | 218 |
| 54 | LG:222335.1:2000SEP08 | 3113348H1 | 10 | 289 |
| 54 | LG:222335.1:2000SEP08 | 702435H1 | 15 | 242 |
| 54 | LG:222335.1:2000SEP08 | 4974222H1 | 18 | 276 |
| 54 | LG:222335.1:2000SEP08 | 2343445H1 | 20 | 268 |
| 54 | LG:222335.1:2000SEP08 | 2343445F6 | 20 | 546 |
| 54 | LG:222335.1:2000SEP08 | 711988H1 | 85 | 306 |
| 54 | LG:222335.1:2000SEP08 | 704169H1 | 85 | 364 |
| 54 | LG:222335.1:2000SEP08 | 337408H1 | 93 | 282 |
| 54 | LG:222335.1:2000SEP08 | 3163266H1 | 155 | 431 |
| 54 | LG:222335.1:2000SEP08 | 4640786H1 | 165 | 437 |
| 54 | LG:222335.1:2000SEP08 | 2343445T6 | 264 | 815 |
| 54 | LG:222335.1:2000SEP08 | 439477H1 | 272 | 514 |
| 54 | LG:222335.1:2000SEP08 | 3778951T6 | 332 | 830 |
| 54 | LG:222335.1:2000SEP08 | 1737725F6 | 401 | 888 |
| 54 | LG:222335.1:2000SEP08 | 1737725H1 | 401 | 608 |
| 54 | LG:222335.1:2000SEP08 | 4001016H1 | 435 | 583 |
| 54 | LG:222335.1:2000SEP08 | g3841204 | 548 | 853 |
| 54 | LG:222335.1:2000SEP08 | 6336726H1 | 1174 | 1420 |
| 54 | LG:222335.1:2000SEP08 | 6850125H1 | 1178 | 1420 |
| 55 | LG:331342.1:2000SEP08 | 3415969F7 | 497 | 980 |
| 55 | LG:331342.1:2000SEP08 | 1003818H1 | 601 | 809 |
| 55 | LG:331342.1:2000SEP08 | 7340365H1 | 601 | 1134 |
| 55 | LG:331342.1:2000SEP08 | 4542316H1 | 633 | 873 |
| 55 | LG:331342.1:2000SEP08 | 4542316F6 | 639 | 1203 |
| 55 | LG:331342.1:2000SEP08 | 4519202H1 | 712 | 975 |
| 55 | LG:331342.1:2000SEP08 | 1272054H1 | 899 | 1126 |
| 55 | LG:331342.1:2000SEP08 | 3966023H1 | 915 | 1057 |
| 55 | LG:331342.1:2000SEP08 | 5316380H1 | 376 | 591 |
| 55 | LG:331342.1:2000SEP08 | 5316222H1 | 376 | 534 |
| 55 | LG:331342.1:2000SEP08 | 4050724H1 | 457 | 746 |
| 55 | LG:331342.1:2000SEP08 | 3415969H1 | 468 | 727 |
| 55 | LG:331342.1:2000SEP08 | 2414808T6 | 1 | 201 |
| 55 | LG:331342.1:2000SEP08 | 194403H1 | 3 | 176 |
| 55 | LG:331342.1:2000SEP08 | 3962146F6 | 8 | 193 |
| 55 | LG:331342.1:2000SEP08 | 2414808F6 | 8 | 441 |
| 55 | LG:331342.1:2000SEP08 | 3962146H1 | 60 | 193 |
| 55 | LG:331342.1:2000SEP08 | 2414808H1 | 191 | 441 |
| 55 | LG:331342.1:2000SEP08 | 7637162H1 | 302 | 876 |
| 55 | LG:331342.1:2000SEP08 | 6606050H1 | 312 | 789 |
| 55 | LG:331342.1:2000SEP08 | 7312830H1 | 361 | 823 |
| 55 | LG:331342.1:2000SEP08 | 5316167H1 | 376 | 584 |
| 55 | LG:331342.1:2000SEP08 | 5318337H1 | 376 | 622 |
| 56 | LG:021770.1:2000SEP08 | 5875430H1 | 597 | 866 |
| 56 | LG:021770.1:2000SEP08 | 718539H1 | 656 | 941 |
| 56 | LG:021770.1:2000SEP08 | 6553139T8 | 594 | 1115 |
| 56 | LG:021770.1:2000SEP08 | 4618506H1 | 590 | 827 |
| 56 | LG:021770.1:2000SEP08 | 7160062H1 | 453 | 950 |
| 56 | LG:021770.1:2000SEP08 | 7603731J1 | 525 | 957 |
| 56 | LG:021770.1:2000SEP08 | 3778516H1 | 433 | 735 |
| 56 | LG:021770.1:2000SEP08 | 4819519H1 | 70 | 340 |
| 56 | LG:021770.1:2000SEP08 | 4970514H1 | 341 | 610 |
| 56 | LG:021770.1:2000SEP08 | 5686262F6 | 57 | 592 |
| 56 | LG:021770.1:2000SEP08 | g2155825 | 66 | 560 |
| 56 | LG:021770.1:2000SEP08 | 4819519F7 | 69 | 652 |
| 56 | LG:021770.1:2000SEP08 | 5754744H1 | 1 | 497 |
| 56 | LG:021770.1:2000SEP08 | 5686262H1 | 43 | 299 |
| 56 | LG:021770.1:2000SEP08 | 5068302H1 | 43 | 315 |
| 56 | LG:021770.1:2000SEP08 | g1442458 | 54 | 265 |
| 56 | LG:021770.1:2000SEP08 | 2477611H1 | 858 | 1061 |
| 56 | LG:021770.1:2000SEP08 | 2477611F7 | 858 | 1292 |
| 56 | LG:021770.1:2000SEP08 | 2477611F6 | 858 | 1333 |
| 56 | LG:021770.1:2000SEP08 | 4313608H1 | 871 | 1193 |
| 56 | LG:021770.1:2000SEP08 | 1461091H1 | 899 | 1137 |
| 56 | LG:021770.1:2000SEP08 | 639781H1 | 945 | 1212 |
| 56 | LG:021770.1:2000SEP08 | 7619831J1 | 953 | 1308 |
| 56 | LG:021770.1:2000SEP08 | 2477611T6 | 1011 | 1575 |
| 56 | LG:021770.1:2000SEP08 | 1967278R6 | 1019 | 1507 |
| 56 | LG:021770.1:2000SEP08 | 1967278H1 | 1020 | 1295 |
| 56 | LG:021770.1:2000SEP08 | 243837H1 | 1071 | 1231 |
| 56 | LG:021770.1:2000SEP08 | 1007489H1 | 1138 | 1433 |
| 56 | LG:021770.1:2000SEP08 | g6472352 | 1247 | 1618 |
| 56 | LG:021770.1:2000SEP08 | 7396616H1 | 1261 | 1773 |
| 56 | LG:021770.1:2000SEP08 | g2155826 | 1268 | 1631 |
| 56 | LG:021770.1:2000SEP08 | g3116842 | 1287 | 1641 |
| 56 | LG:021770.1:2000SEP08 | g709667 | 1293 | 1623 |
| 56 | LG:021770.1:2000SEP08 | g2100629 | 1294 | 1624 |
| 56 | LG:021770.1:2000SEP08 | g816343 | 1352 | 1619 |
| 56 | LG:021770.1:2000SEP08 | g816686 | 1358 | 1619 |
| 56 | LG:021770.1:2000SEP08 | g670313 | 1362 | 1610 |
| 56 | LG:021770.1:2000SEP08 | g682772 | 1366 | 1610 |
| 56 | LG:021770.1:2000SEP08 | 7756477J1 | 1399 | 1997 |
| 56 | LG:021770.1:2000SEP08 | 2615849H1 | 1419 | 1605 |
| 56 | LG:021770.1:2000SEP08 | 5259534H1 | 1463 | 1620 |
| 56 | LG:021770.1:2000SEP08 | g2719096 | 1552 | 1630 |
| 56 | LG:021770.1:2000SEP08 | 3483806F6 | 1564 | 2025 |
| 56 | LG:021770.1:2000SEP08 | 3483806H1 | 1564 | 1742 |
| 56 | LG:021770.1:2000SEP08 | 3187717T6 | 1586 | 1971 |
| 56 | LG:021770.1:2000SEP08 | 3352115H1 | 1659 | 1749 |
| 56 | LG:021770.1:2000SEP08 | 1812178H1 | 1713 | 1979 |
| 56 | LG:021770.1:2000SEP08 | 7756477H1 | 1781 | 2056 |
| 56 | LG:021770.1:2000SEP08 | 5851722H1 | 1851 | 2056 |
| 56 | LG:021770.1:2000SEP08 | 3187717R6 | 1876 | 2056 |
| 56 | LG:021770.1:2000SEP08 | 1967278T6 | 1884 | 2471 |
| 56 | LG:021770.1:2000SEP08 | 4187256H1 | 1909 | 2057 |
| 56 | LG:021770.1:2000SEP08 | 4184364H1 | 1914 | 2132 |
| 56 | LG:021770.1:2000SEP08 | 3483806T6 | 1931 | 2478 |
| 56 | LG:021770.1:2000SEP08 | 7250489H1 | 1945 | 2497 |
| 56 | LG:021770.1:2000SEP08 | 7250522H1 | 1945 | 2491 |
| 56 | LG:021770.1:2000SEP08 | 5686262T6 | 1952 | 2470 |
| 56 | LG:021770.1:2000SEP08 | 3187717H1 | 1983 | 2044 |
| 56 | LG:021770.1:2000SEP08 | 2570543H1 | 2327 | 2515 |
| 56 | LG:021770.1:2000SEP08 | 2570543R6 | 2327 | 2515 |
| 56 | LG:021770.1:2000SEP08 | 2570543T6 | 2327 | 2477 |
| 56 | LG:021770.1:2000SEP08 | 5958482H1 | 2341 | 2503 |
| 56 | LG:021770.1:2000SEP08 | 1618155T6 | 2368 | 2477 |
| 56 | LG:021770.1:2000SEP08 | 4692935H1 | 2368 | 2420 |
| 56 | LG:021770.1:2000SEP08 | g6470601 | 2368 | 2522 |
| 56 | LG:021770.1:2000SEP08 | 1732834H1 | 2373 | 2450 |
| 56 | LG:021770.1:2000SEP08 | g2782960 | 2445 | 2581 |
| 56 | LG:021770.1:2000SEP08 | 6100740H1 | 2452 | 2643 |
| 56 | LG:021770.1:2000SEP08 | 3249668T6 | 2561 | 3105 |
| 56 | LG:021770.1:2000SEP08 | 3249668F6 | 2813 | 3082 |
| 56 | LG:021770.1:2000SEP08 | 3249668H1 | 2932 | 3082 |
| 57 | LG:181607.9:2000SEP08 | 7344412H1 | 37 | 548 |
| 57 | LG:181607.9:2000SEP08 | 7616670H1 | 40 | 579 |
| 57 | LG:181607.9:2000SEP08 | 3382088H1 | 71 | 302 |
| 57 | LG:181607.9:2000SEP08 | 3622354H1 | 71 | 323 |
| 57 | LG:181607.9:2000SEP08 | 3810979H1 | 79 | 382 |
| 57 | LG:181607.9:2000SEP08 | 6314189F8 | 80 | 762 |
| 57 | LG:181607.9:2000SEP08 | 4763755H1 | 93 | 271 |
| 57 | LG:181607.9:2000SEP08 | g1317224 | 95 | 516 |
| 57 | LG:181607.9:2000SEP08 | 2734055H1 | 105 | 366 |
| 57 | LG:181607.9:2000SEP08 | 2986979H1 | 107 | 350 |
| 57 | LG:181607.9:2000SEP08 | g885028 | 221 | 484 |
| 57 | LG:181607.9:2000SEP08 | g885016 | 222 | 578 |
| 57 | LG:181607.9:2000SEP08 | 3735039H1 | 266 | 504 |
| 57 | LG:181607.9:2000SEP08 | 3199148H1 | 1 | 268 |
| 58 | LG:1042768.1:2000SEP08 | 6798443H1 | 1 | 438 |
| 58 | LG: 1042768.1:2000SEP08 | 6790795F8 | 1 | 602 |
| 58 | LG:1042768.1:2000SEP08 | 6790795H1 | 1 | 529 |
| 58 | LG:1042768.1:2000SEP08 | 6798443F8 | 1 | 618 |
| 58 | LG: 1042768.1:2000SEP08 | 6790795T8 | 244 | 850 |
| 59 | LG:282729.1:2000SEP08 | 4365538F6 | 1 | 292 |
| 59 | LG:282729.1:2000SEP08 | 4365538H1 | 1 | 261 |
| 59 | LG:282729.1:2000SEP08 | 6810113J1 | 11 | 398 |
| 59 | LG:282729.1:2000SEP08 | 6810113H1 13 | 399 | |
| 59 | LG:282729.1:2000SEP08 | 5137624H1 | 248 | 353 |
| 59 | LG:282729.1:2000SEP08 | g4069212 | 253 | 704 |
| 59 | LG:282729.1:2000SEP08 | 5137624T6 | 354 | 664 |
| 60 | LG:998305.3:2000SEP08 | 3591650H1 | 1 | 282 |
| 60 | LG:998305.3:2000SEP08 | 3651255H1 | 65 | 362 |
| 60 | LG:998305.3:2000SEP08 | 3651255F6 | 66 | 617 |
| 60 | LG:998305.3:2000SEP08 | 3462213H1 | 477 | 742 |
| 60 | LG:998305.3:2000SEP08 | 3651 255T6 | 577 | 927 |
| 60 | LG:998305.3:2000SEP08 | 1853882H1 | 669 | 803 |
| 60 | LG:998305.3:2000SEP08 | 1854687H1 | 669 | 962 |
| 60 | LG:998305.3:2000SEP08 | 1854687F6 | 669 | 956 |
| 60 | LG:998305.3:2000SEP08 | 2244779H1 | 776 | 928 |
| 61 | LG:1135213.1:2000SEP08 | 4145560H1 | 1 | 337 |
| 61 | LG:1135213.1:2000SEP08 | 7182979H1 | 1 | 537 |
| 61 | LG:1135213.1:2000SEP08 | g4929686 | 1 | 1581 |
| 61 | LG:1135213.1:2000SEP08 | g1881193 | 113 | 359 |
| 61 | LG:1135213.1:2000SEP08 | 798770H1 | 206 | 449 |
| 61 | LG:1135213.1:2000SEP08 | g1198695 | 214 | 498 |
| 61 | LG:1135213.1:2000SEP08 | g1637735 | 380 | 642 |
| 61 | LG:1135213.1:2000SEP08 | g2204679 | 39 | 511 |
| 61 | LG:1135213.1:2000SEP08 | 5540595H1 | 1 | 195 |
| 61 | LG:1135213.1:2000SEP08 | g1970769 | 1 | 345 |
| 61 | LG:1135213.1:2000SEP08 | g1970753 | 1 | 325 |
| 61 | LG:1135213.1:2000SEP08 | g1971048 | 1 | 253 |
| 61 | LG:I135213.1:2000SEP08 | g1970777 | 1 | 223 |
| 61 | LG:1135213.1:2000SEP08 | g815792 | 8 | 284 |
| 61 | LG:1135213.1:2000SEP08 | g1441646 | 3 | 303 |
| 61 | LG:1135213.1:2000SEP08 | g4372035 | 14 | 479 |
| 61 | LG:1135213.1:2000SEP08 | g2930515 | 35 | 487 |
| 61 | LG:1135213.1:2000SEP08 | g4897951 | 44 | 477 |
| 61 | LG:1135213.1:2000SEP08 | 609028H1 | 27 | 178 |
| 61 | LG:1135213.1:2000SEP08 | g2782816 | 15 | 417 |
| 61 | LG:1135213.1:2000SEP08 | g4326525 | 1 | 141 |
| 61 | LG:1135213.1:2000SEP08 | g2525795 | 28 | 236 |
| 62 | LG:267762.1:2000SEP08 | 3941090F8 | 1 | 334 |
| 62 | LG:267762.1:2000SEP08 | 1704531H1 | 153 | 292 |
| 62 | LG:267762.1:2000SEP08 | 7658244J1 | 267 | 830 |
| 62 | LG:267762.1:2000SEP08 | 5674936F6 | 283 | 888 |
| 62 | LG:267762.1:2000SEP08 | 5674936H1 | 283 | 545 |
| 62 | LG:267762.1:2000SEP08 | 5785070H1 | 482 | 768 |
| 62 | LG:267762.1:2000SEP08 | 2831361H1 | 514 | 783 |
| 62 | LG:267762.1:2000SEP08 | g1523057 | 527 | 994 |
| 62 | LG:267762.1:2000SEP08 | 6244881H1 | 600 | 677 |
| 62 | LG:267762.1:2000SEP08 | 3731660H1 | 675 | 978 |
| 62 | LG:267762.1:2000SEP08 | g1950270 | 777 | 1103 |
| 62 | LG:267762.1:2000SEP08 | 5674936T6 | 782 | 1313 |
| 62 | LG:267762.1:2000SEP08 | 7762849J1 | 824 | 1474 |
| 62 | LG:267762.1:2000SEP08 | g1523013 | 882 | 1063 |
| 62 | LG:267762.1:2000SEP08 | g3678477 | 895 | 1352 |
| 62 | LG:267762.1:2000SEP08 | 2797262H1 | 986 | 1231 |
| 62 | LG:267762.1:2000SEP08 | 2797262F6 | 986 | 1555 |
| 62 | LG:267762.1:2000SEP08 | 3732853H1 | 1059 | 1205 |
| 62 | LG:267762.1:2000SEP08 | g766967 | 1146 | 1503 |
| 62 | LG:267762.1:2000SEP08 | 2797262T6 | 1199 | 1645 |
| 62 | LG:267762.1:2000SEP08 | 6768646J1 | 1221 | 1437 |
| 62 | LG:267762.1:2000SEP08 | 2561680H1 | 1367 | 1657 |
| 62 | LG:267762.1:2000SEP08 | g3750627 | 1401 | 1645 |
| 62 | LG:267762.1:2000SEP08 | g2557941 | 1510 | 1645 |
| 63 | LG:120744.1:2000SEP08 | 5643301H1 | 1 | 266 |
| 63 | LG:120744.1:2000SEP08 | 5643301F6 | 1 | 412 |
| 63 | LG:120744.1:2000SEP08 | 7007819H1 | 12 | 557 |
| 63 | LG:120744.1:2000SEP08 | 7161666H1 | 34 | 435 |
| 63 | LG:120744.1:2000SEP08 | 7008530H1 | 19 | 463 |
| 63 | LG:120744.1:2000SEP08 | 7161666F8 | 34 | 651 |
| 63 | LG:120744.1:2000SEP08 | 7017571H1 | 42 | 211 |
| 63 | LG:120744.1:2000SEP08 | 7017571F8 | 41 | 656 |
| 63 | LG:120744.1:2000SEP08 | 3277305H1 | 128 | 393 |
| 63 | LG:120744.1:2000SEP08 | 3277305F6 | 138 | 728 |
| 63 | LG:120744.1:2000SEP08 | 3565182H1 | 492 | 782 |
| 63 | LG:120744.1:2000SEP08 | 5643301R8 | 940 | 1116 |
| 63 | LG:120744.1:2000SEP08 | g4293857 | 672 | 947 |
| 63 | LG:120744.1:2000SEP08 | 7161666R8 | 707 | 1169 |
| 63 | LG:120744.1:2000SEP08 | 2992317H1 | 816 | 1098 |
| 63 | LG:120744.1:2000SEP08 | 5643301R6 | 823 | 1115 |
| 64 | LG:403409.1:2000SEP08 | 5643224H1 | 22 | 276 |
| 64 | LG:403409.1:2000SEP08 | 91998848 | 22 | 296 |
| 64 | LG:403409.1:2000SEP08 | 3126704H1 | 22 | 298 |
| 64 | LG:403409.1:2000SEP08 | 3126978F6 | 22 | 611 |
| 64 | LG:403409.1:2000SEP08 | 3126704F6 | 22 | 600 |
| 64 | LG:403409.1:2000SEP08 | 3256088H1 | 24 | 259 |
| 64 | LG:403409.1:2000SEP08 | 1728945H1 | 26 | 246 |
| 64 | LG:403409.1:2000SEP08 | 6141236H1 | 28 | 370 |
| 64 | LG:403409.1:2000SEP08 | 6141236F8 | 28 | 633 |
| 64 | LG:403409.1:2000SEP08 | 6328822H1 | 47 | 592 |
| 64 | LG:403409.1:2000SEP08 | 3388023H1 | 89 | 361 |
| 64 | LG:403409.1:2000SEP08 | 6134014H1 | 110 | 401 |
| 64 | LG:403409.1:2000SEP08 | 3323733H1 | 192 | 461 |
| 64 | LG:403409.1:2000SEP08 | 3409854H1 | 357 | 618 |
| 64 | LG:403409.1:2000SEP08 | 4068325F6 | 515 | 1108 |
| 64 | LG:403409.1:2000SEP08 | 4068325H1 | 517 | 804 |
| 64 | LG:403409.1:2000SEP08 | 3629455H1 | 560 | 835 |
| 64 | LG:403409.1:2000SEP08 | 5499626H1 | 1 | 255 |
| 64 | LG:403409.1:2000SEP08 | 5500026H1 | 1 | 161 |
| 64 | LG:403409.1:2000SEP08 | 5500309H1 | 1 | 227 |
| 64 | LG:403409.1:2000SEP08 | 5499909H1 | 1 | 206 |
| 64 | LG:403409.1:2000SEP08 | 2723940H1 | 1 | 243 |
| 64 | LG:403409.1:2000SEP08 | 2723940F6 | 1 | 333 |
| 64 | LG:403409.1:2000SEP08 | 3751233H1 | 577 | 867 |
| 64 | LG:403409.1:2000SEP08 | 5812189H1 | 644 | 840 |
| 64 | LG:403409.1:2000SEP08 | 5812190H1 | 644 | 845 |
| 64 | LG:403409.1:2000SEP08 | 311752H1 | 657 | 744 |
| 64 | LG:403409.1:2000SEP08 | 373638H1 | 663 | 880 |
| 64 | LG:403409.1:2000SEP08 | 7377730H1 | 697 | 1236 |
| 64 | LG:403409.1:2000SEP08 | 6333709H1 | 705 | 1240 |
| 64 | LG:403409.1:2000SEP08 | 6329688H1 | 705 | 1325 |
| 64 | LG:403409.1:2000SEP08 | 7080559H1 | 760 | 1170 |
| 64 | LG:403409.1:2000SEP08 | 6532643H1 | 803 | 1384 |
| 64 | LG:403409.1:2000SEP08 | g6570650 | 881 | 1310 |
| 64 | LG:403409.1:2000SEP08 | 6141236T8 | 989 | 1579 |
| 64 | LG:403409.1:2000SEP08 | 4068325T6 | 1018 | 1641 |
| 64 | LG:403409.1:2000SEP08 | 3126704T6 | 1056 | 1580 |
| 64 | LG:403409.1:2000SEP08 | 7068876H1 | 1069 | 1483 |
| 64 | LG:403409.1:2000SEP08 | 3256362H1 | 1308 | 1554 |
| 64 | LG:403409.1:2000SEP08 | g1400213 | 1337 | 1682 |
| 64 | LG:403409.1:2000SEP08 | 93254782 | 1346 | 1682 |
| 64 | LG:403409.1:2000SEP08 | g1383466 | 1395 | 1696 |
| 64 | LG:403409.1:2000SEP08 | 6412487H1 | 1494 | 1931 |
| 64 | LG:403409.1:2000SEP08 | 5534143H1 | 1578 | 1820 |
| 64 | LG:403409.1:2000SEP08 | 7468129H1 | 1672 | 2123 |
| 64 | LG:403409.1:2000SEP08 | 6869946H1 | 1717 | 2275 |
| 64 | LG:403409.1:2000SEP08 | g4510725 | 1831 | 2272 |
| 64 | LG:403409.1:2000SEP08 | 6298042H1 | 1871 | 2146 |
| 64 | LG:403409.1:2000SEP08 | g1998847 | 1973 | 2275 |
| 64 | LG:403409.1:2000SEP08 | 3541104H1 | 1991 | 2272 |
| 65 | LG:226874.3:2000SEP08 | 6435270H1 | 1 | 397 |
| 65 | LG:226874.3:2000SEP08 | 6431679H1 | 1 | 485 |
| 65 | LG:226874.3:2000SEP08 | 6427109H1 | 1 | 445 |
| 65 | LG:226874.3:2000SEP08 | 6779705H1 | 178 | 713 |
| 65 | LG:226874.3:2000SEP08 | 5767442F8 | 178 | 779 |
| 65 | LG:226874.3:2000SEP08 | 5767442H1 | 316 | 780 |
| 65 | LG:226874.3:2000SEP08 | 6779705R8 | 335 | 929 |
| 65 | LG:226874.3:2000SEP08 | 6779705J1 | 335 | 933 |
| 65 | LG:226874.3:2000SEP08 | 7198301H1 | 693 | 1099 |
| 65 | LG:226874.3:2000SEP08 | 6536795H1 | 895 | 1461 |
| 65 | LG:226874.3:2000SEP08 | g2071279 | 927 | 1231 |
| 65 | LG:226874.3:2000SEP08 | g3802687 | 950 | 1338 |
| 65 | LG:226874.3:2000SEP08 | 7264450H1 | 1077 | 1547 |
| 65 | LG:226874.3:2000SEP08 | 6246443H1 | 1364 | 1871 |
| 66 | LG:1045521.4:2000SEP08 | g4453997 | 3410 | 3766 |
| 66 | LG:1045521.4:2000SEP08 | g5436696 | 3402 | 3773 |
| 66 | LG:1045521.4:2000SEP08 | g4266778 | 3401 | 3775 |
| 66 | LG:1045521.4:2000SEP08 | g3174193 | 3398 | 3774 |
| 66 | LG:1045521.4:2000SEP08 | 4136385H1 | 3545 | 3829 |
| 66 | LG:1045521.4:2000SEP08 | 94195581 | 3607 | 3774 |
| 66 | LG:1045521.4:2000SEP08 | g4438434 | 3604 | 3773 |
| 66 | LG:1045521.4:2000SEP08 | g4392683 | 3394 | 3775 |
| 66 | LG:1045521.4:2000SEP08 | g5745081 | 3397 | 3777 |
| 66 | LG:1045521.4:2000SEP08 | 2761101H1 | 3369 | 3598 |
| 66 | LG:1045521.4:2000SEP08 | 2816971H1 | 3378 | 3641 |
| 66 | LG:1045521.4:2000SEP08 | g3756033 | 3380 | 3775 |
| 66 | LG:1045521.4:2000SEP08 | g3094407 | 3385 | 3679 |
| 66 | LG:1045521.4:2000SEP08 | 2189244H1 | 3390 | 3636 |
| 66 | LG:1045521.4:2000SEP08 | g1376982 | 3338 | 3766 |
| 66 | LG:1045521.4:2000SEP08 | 7247994H1 | 3343 | 3773 |
| 66 | LG:1045521.4:2000SEP08 | g4223122 | 3345 | 3775 |
| 66 | LG:1045521.4:2000SEP08 | g3839512 | 3351 | 3775 |
| 66 | LG:1045521.4:2000SEP08 | g5887631 | 3350 | 3775 |
| 66 | LG:1045521.4:2000SEP08 | g5855693 | 3351 | 3776 |
| 66 | LG:1045521.4:2000SEP08 | g4394054 | 3354 | 3773 |
| 66 | LG:1045521.4:2000SEP08 | g5743698 | 3363 | 3775 |
| 66 | LG:1045521.4:2000SEP08 | 2112746T6 | 3317 | 3735 |
| 66 | LG:1045521.4:2000SEP08 | 1813318T6 | 3319 | 3739 |
| 66 | LG:1045521.4:2000SEP08 | g5673655 | 3323 | 3775 |
| 66 | LG:1045521.4:2000SEP08 | 1813318F6 | 3326 | 3657 |
| 66 | LG:1045521.4:2000SEP08 | 1813318H1 | 3326 | 3558 |
| 66 | LG:1045521.4:2000SEP08 | g4309999 | 3332 | 3776 |
| 66 | LG:1045521.4:2000SEP08 | g5837582 | 3338 | 3781 |
| 66 | LG:1045521.4:2000SEP08 | g694185 | 3465 | 3787 |
| 66 | LG:1045521.4:2000SEP08 | g566999 | 3478 | 3773 |
| 66 | LG:1045521.4:2000SEP08 | 2082531T6 | 3413 | 3734 |
| 66 | LG:1045521.4:2000SEP08 | g5934167 | 3415 | 3778 |
| 66 | LG:1045521.4:2000SEP08 | g5365380 | 3415 | 3775 |
| 66 | LG:1045521.4:2000SEP08 | g1194703 | 3414 | 3773 |
| 66 | LG:1045521.4:2000SEP08 | g2348619 | 3414 | 3775 |
| 66 | LG:1045521.4:2000SEP08 | g5675116 | 3415 | 3776 |
| 66 | LG:1045521.4:2000SEP08 | g2584365 | 3416 | 3775 |
| 66 | LG:1045521.4:2000SEP08 | g2953747 | 3417 | 3777 |
| 66 | LG:1045521.4:2000SEP08 | g2264833 | 3419 | 3775 |
| 66 | LG:1045521.4:2000SEP08 | g5152477 | 3424 | 3753 |
| 66 | LG:1045521.4:2000SEP08 | 951625H1 | 3450 | 3682 |
| 66 | LG:1045521.4:2000SEP08 | 3016423H1 | 3453 | 3753 |
| 66 | LG:1045521.4:2000SEP08 | g6474480 | 3316 | 3775 |
| 66 | LG:1045521.4:2000SEP08 | 7705621H1 | 1924 | 2521 |
| 66 | LG:1045521.4:2000SEP08 | 1287084H1 | 3313 | 3446 |
| 66 | LG:1045521.4:2000SEP08 | 7022070H1 | 564 | 1144 |
| 66 | LG:1045521.4:2000SEP08 | g5444114 | 3311 | 3775 |
| 66 | LG:1045521.4:2000SEP08 | 92059013 | 3307 | 3778 |
| 66 | LG:1045521.4:2000SEP08 | 1994552T6 | 3312 | 3734 |
| 66 | LG:1045521.4:2000SEP08 | 1287084F1 | 3313 | 3780 |
| 66 | LG:1045521.4:2000SEP08 | 1757131H1 | 3095 | 3314 |
| 66 | LG:1045521.4:2000SEP08 | 3121994H1 | 3121 | 3398 |
| 66 | LG:1045521.4:2000SEP08 | 2681674H1 | 3126 | 3419 |
| 66 | LG:1045521.4:2000SEP08 | 1710178H1 | 3140 | 3328 |
| 66 | LG:1045521.4:2000SEP08 | 2706109T6 | 3161 | 3735 |
| 66 | LG:1045521.4:2000SEP08 | 2607376T6 | 3175 | 3735 |
| 66 | LG:1045521.4:2000SEP08 | 1398469H1 | 3181 | 3415 |
| 66 | LG:1045521.4:2000SEP08 | 1686019T6 | 3196 | 3736 |
| 66 | LG:1045521.4:2000SEP08 | 2480005H1 | 3202 | 3457 |
| 66 | LG:1045521.4:2000SEP08 | 2480053H1 | 3202 | 3471 |
| 66 | LG:1045521.4:2000SEP08 | 1414724H1 | 3219 | 3452 |
| 66 | LG:1045521.4:2000SEP08 | 3966031H1 | 3221 | 3365 |
| 66 | LG:1045521.4:2000SEP08 | 3965562H1 | 3221 | 3511 |
| 66 | LG:1045521.4:2000SEP08 | 2419122T6 | 3256 | 3736 |
| 66 | LG:1045521.4:2000SEP08 | 6315349H1 | 3283 | 3774 |
| 66 | LG:1045521.4:2000SEP08 | 6315249H1 | 3283 | 3773 |
| 66 | LG:1045521.4:2000SEP08 | 1863643T6 | 3285 | 3720 |
| 66 | LG:1045521.4:2000SEP08 | g3152117 | 3298 | 3778 |
| 66 | LG:1045521.4:2000SEP08 | 7390568H2 | 2944 | 3392 |
| 66 | LG:1045521.4:2000SEP08 | 7746832H1 | 2160 | 2715 |
| 66 | LG:1045521.4:2000SEP08 | 7712386J1 | 1330 | 1972 |
| 66 | LG:1045521.4:2000SEP08 | 7433684H1 | 176 | 707 |
| 66 | LG:1045521.4:2000SEP08 | 5100939H1 | 3264 | 3502 |
| 66 | LG:1045521.4:2000SEP08 | g2291354 | 3519 | 3778 |
| 66 | LG:1045521.4:2000SEP08 | g4685761 | 3523 | 3782 |
| 66 | LG:1045521.4:2000SEP08 | g6703923 | 3374 | 3775 |
| 66 | LG:1045521.4:2000SEP08 | 741995H1 | 3321 | 3564 |
| 66 | LG:1045521.4:2000SEP08 | g1349221 | 3264 | 3574 |
| 66 | LG:1045521.4:2000SEP08 | 4731668H1 | 3257 | 3328 |
| 66 | LG:1Q45521.4:2000SEP08 | 6256785H1 | 2960 | 3168 |
| 66 | LG:1045521.4:2000SEP08 | 6892739H1 | 2973 | 3373 |
| 66 | LG:1045521,4:2000SEP08 | 3016905H1 | 3022 | 3315 |
| 66 | LG:1045521.4:2000SEP08 | 1740793H1 | 3024 | 3238 |
| 66 | LG:1045521.4:2000SEP08 | 1296544F6 | 3049 | 3351 |
| 66 | LG:1045521.4:2000SEP08 | 1296544H1 | 3049 | 3278 |
| 66 | LG:1045521.4:2000SEP08 | 1292654H1 | 3049 | 3284 |
| 66 | LG:1045521.4:2000SEP08 | 1294126H1 | 3049 | 3299 |
| 66 | LG:1045521.4:2000SEP08 | 5833956H1 | 3052 | 3307 |
| 66 | LG:1045521.4:2000SEP08 | 3788784H1 | 3070 | 3349 |
| 66 | LG:1045521.4:2000SEP08 | 995376H1 | 3072 | 3391 |
| 66 | LG:1045521.4:2000SEP08 | 1863643H1 | 3088 | 3316 |
| 66 | LG:1045521.4:2000SEP08 | 1863643F6 | 3088 | 3461 |
| 66 | LG:1045521.4:2000SEP08 | 1296544T6 | 3095 | 3734 |
| 66 | LG:1045521.4:2000SEP08 | 7046394H1 | 2742 | 3279 |
| 66 | LG:1045521.4:2000SEP08 | 6764773H1 | 2755 | 3041 |
| 66 | LG:1045521.4:2000SEP08 | 5519077H1 | 2812 | 3075 |
| 66 | LG:1045521.4:2000SEP08 | 6838807H1 | 2821 | 3194 |
| 66 | LG:1045521.4:2000SEP08 | 6250284H1 | 2833 | 2892 |
| 66 | LG:1045521.4:2000SEP08 | g1625167 | 2838 | 3155 |
| 66 | LG:1045521.4:2000SEP08 | g1615333 | 2853 | 3261 |
| 66 | LG:1045521.4:2000SEP08 | 4527137H1 | 2854 | 31H |
| 66 | LG:1045521.4:2000SEP08 | 6498605H1 | 2871 | 3360 |
| 66 | LG:1045521.4:2000SEP08 | 1809783H1 | 2875 | 3103 |
| 66 | LG:1045521.4:2000SEP08 | 1809783F6 | 2875 | 3198 |
| 66 | LG:1045521.4:2000SEP08 | 4447331H1 | 2879 | 3138 |
| 66 | LG:1045521.4:2000SEP08 | 2307638H1 | 2888 | 3118 |
| 66 | LG:1045521.4:2000SEP08 | 3784937W | 2891 | 3195 |
| 66 | LG:1045521.4:2000SEP08 | 2985814H1 | 2895 | 3187 |
| 66 | LG:1045521.4:2000SEP08 | g694351 | 2908 | 3206 |
| 66 | LG:1045521.4:2000SEP08 | 6414125H1 | 2911 | 3103 |
| 66 | LG:1045521.4:2000SEP08 | 2082531F6 | 2389 | 2749 |
| 66 | LG:1045521.4:2000SEP08 | 2607376H1 | 2408 | 2669 |
| 66 | LG:1045521.4:2000SEP08 | 3782031H1 | 2428 | 2729 |
| 66 | LG:1045521.4:2000SEP08 | 5053149H1 | 2431 | 2656 |
| 66 | LG:1045521.4:2000SEP08 | 3351162H1 | 2452 | 2740 |
| 66 | LG:1045521.4:2000SEP08 | 2824756H1 | 2470 | 2768 |
| 66 | LG:1045521.4:2000SEP08 | 6818820H1 | 2484 | 3041 |
| 66 | LG:1045521.4:2000SEP08 | 5204860H1 | 2485 | 2726 |
| 66 | LG:1045521.4:2000SEP08 | 5758032H1 | 2526 | 2791 |
| 66 | LG:1045521.4:2000SEP08 | 3016811H1 | 2529 | 2713 |
| 66 | LG:1045521.4:2000SEP08 | 5262607H2 | 2595 | 2816 |
| 66 | LG:1045521.4:2000SEP08 | 5515948H1 | 2602 | 2872 |
| 66 | LG:1045521.4:2000SEP08 | 1994552R6 | 2636 | 2897 |
| 66 | LG:1045521.4:2000SEP08 | 1994552H1 | 2636 | 2874 |
| 66 | LG:1045521.4:2000SEP08 | 3760435H1 | 2649 | 2951 |
| 66 | LG:1045521.4:2000SEP08 | 2108625H1 | 2681 | 2933 |
| 66 | LG:1045521.4:2000SEP08 | 5487653H1 | 2689 | 2932 |
| 66 | LG:1045521.4:2000SEP08 | 7281395H1 | 2358 | 2924 |
| 66 | LG:1045521.4:2000SEP08 | g2055131 | 2385 | 2873 |
| 66 | LG:1045521.4:2000SEP08 | 2082531H1 | 2389 | 2539 |
| 66 | LG:1045521.4:2000SEP08 | 6892739J1 | 2344 | 2873 |
| 66 | LG:1045521.4:2000SEP08 | g3179156 | 3616 | 3775 |
| 66 | LG:1045521.4:2000SEP08 | 6358626H1 | 3662 | 3773 |
| 66 | LG:1045521.4:2000SEP08 | 4988766H1 | 1783 | 1921 |
| 66 | LG:1045521.4:2000SEP08 | 2634944H1 | 1801 | 2030 |
| 66 | LG:1045521.4:2000SEP08 | 4501131H2 | 1851 | 2045 |
| 66 | LG:1045521.4:2000SEP08 | 3172451H1 | 1897 | 2167 |
| 66 | LG:1045521.4:2000SEP08 | 6715267H1 | 2007 | 2540 |
| 66 | LG:1045521.4:2000SEP08 | 5969546H1 | 2031 | 2540 |
| 66 | LG:1045521.4:2000SEP08 | 3833465H1 | 2076 | 2301 |
| 66 | LG:1045521.4:2000SEP08 | 5656796H1 | 2131 | 2203 |
| 66 | LG:1045521.4:2000SEP08 | 5656764H1 | 2131 | 2395 |
| 66 | LG:1045521.4:2000SEP08 | 4819795H1 | 2146 | 2382 |
| 66 | LG:1045521.4:2000SEP08 | 5072388H1 | 2173 | 2439 |
| 66 | LG:1045521.4:2000SEP08 | 6592270H1 | 2173 | 2638 |
| 66 | LG:1045521.4:2000SEP08 | 2419122H1 | 2181 | 2419 |
| 66 | LG:1045521.4:2000SEP08 | 2419122F6 | 2181 | 2597 |
| 66 | LG:1045521.4:2000SEP08 | 4711930H1 | 2205 | 2377 |
| 66 | LG:1045521.4:2000SEP08 | g574765 | 2210 | 2539 |
| 66 | LG:1045521.4:2000SEP08 | 5661003H1 | 2328 | 2600 |
| 66 | LG:1045521.4:2000SEP08 | 4416117H1 | 1630 | 1866 |
| 66 | LG:1045521.4:2000SEP08 | 860978H1 | 1656 | 1920 |
| 66 | LG:1045521.4:2000SEP08 | 4864552H1 | 1659 | 1919 |
| 66 | LG:1045521.4:2000SEP08 | g3899491 | 1663 | 1919 |
| 66 | LG:1045521.4:2000SEP08 | g1668042 | 1666 | 1919 |
| 66 | LG:1045521.4:2000SEP08 | g2159252 | 1668 | 1928 |
| 66 | LG:1045521.4:2000SEP08 | 5576662H1 | 1676 | 1917 |
| 66 | LG:1045521.4:2000SEP08 | g1815410 | 1692 | 1920 |
| 66 | LG:1045521.4:2000SEP08 | g518911 | 1702 | 1919 |
| 66 | LG:1045521.4:2000SEP08 | 3831968H1 | 1708 | 1909 |
| 66 | LG:1045521.4:2000SEP08 | 859807H1 | 1715 | 1921 |
| 66 | LG:1045521.4:2000SEP08 | g1779186 | 1723 | 1931 |
| 66 | LG:1045521.4:2000SEP08 | g2810009 | 1729 | 1921 |
| 66 | LG:1045521.4:2000SEP08 | g4195367 | 1735 | 1919 |
| 66 | LG:1045521.4:2000SEP08 | g5838814 | 1737 | 1916 |
| 66 | LG:1045521.4:2000SEP08 | g5639161 | 1743 | 1919 |
| 66 | LG:1045521.4:2000SEP08 | g4565722 | 1748 | 1919 |
| 66 | LG:1045521.4:2000SEP08 | 1688229H1 | 1750 | 1919 |
| 66 | LG:1045521.4:2000SEP08 | 5070672H1 | 1771 | 2035 |
| 66 | LG:1045521.4:2000SEP08 | 4643788H1 | 1785 | 2016 |
| 66 | LG:1045521.4:2000SEP08 | 1347561H1 | 1482 | 1706 |
| 66 | LG:1045521.4:2000SEP08 | g3750889 | 1499 | 1921 |
| 66 | LG:1045521.4:2000SEP08 | g824104 | 1513 | 1930 |
| 66 | LG:1045521.4:2000SEP08 | g3753691 | 1514 | 1919 |
| 66 | LG:1045521.4:2000SEP08 | g1692198 | 1517 | 1893 |
| 66 | LG:1045521.4:2000SEP08 | g5438003 | 1522 | 1919 |
| 66 | LG:1045521.4:2000SEP08 | g3411980 | 1523 | 1921 |
| 66 | LG:1045521.4:2000SEP08 | g3412789 | 1527 | 1928 |
| 66 | LG:1045521.4:2000SEP08 | g2354546 | 1528 | 1893 |
| 66 | LG:1045521.4:2000SEP08 | g4982748 | 1534 | 1926 |
| 66 | LG:1045521.4:2000SEP08 | g4851706 | 1535 | 1920 |
| 66 | LG:1045521.4:2000SEP08 | 3014352H1 | 1536 | 1841 |
| 66 | LG:1045521.4:2000SEP08 | g5659087 | 1539 | 1921 |
| 66 | LG:1045521.4:2000SEP08 | 2284667H1 | 1547 | 1766 |
| 66 | LG:1045521.4:2000SEP08 | g3155655 | 1555 | 1921 |
| 66 | LG:1045521.4:2000SEP08 | 1688229F6 | 1558 | 1919 |
| 66 | LG:1045521.4:2000SEP08 | g4107912 | 1577 | 1919 |
| 66 | LG:1045521.4:2000SEP08 | g5879310 | 1596 | 1920 |
| 66 | LG:1045521.4:2000SEP08 | g1648498 | 1606 | 1919 |
| 66 | LG:1045521.4:2000SEP08 | g395479 | 1613 | 1921 |
| 66 | LG:1045521.4:2000SEP08 | 2378339H1 | 1628 | 1862 |
| 66 | LG:1045521.4:2000SEP08 | 7712386H1 | 1611 | 2256 |
| 66 | LG:1045521.4:2000SEP08 | 5284064H1 | 1460 | 1580 |
| 66 | LG:1045521.4:2000SEP08 | 2643406T6 | 1457 | 1878 |
| 66 | LG:1045521.4:2000SEP08 | 6630849H1 | 1476 | 1921 |
| 66 | LG:1045521.4:2000SEP08 | 7340290H1 | 1192 | 1771 |
| 66 | LG:1045521.4:2000SEP08 | g4327763 | 3512 | 3775 |
| 66 | LG:1045521.4:2000SEP08 | 2301252H2 | 3493 | 3666 |
| 66 | LG:1045521.4:2000SEP08 | 2510130H1 | 3513 | 3734 |
| 66 | LG:1045521.4:2000SEP08 | g5671827 | 3479 | 3773 |
| 66 | LG:1045521.4:2000SEP08 | g3677607 | 3479 | 3773 |
| 66 | LG:1045521.4:2000SEP08 | g3755042 | 3481 | 3775 |
| 66 | LG:1045521.4:2000SEP08 | 5102917H1 | 3489 | 3763 |
| 66 | LG:1045521.4:2000SEP08 | g3755759 | 3486 | 3776 |
| 66 | LG:1045521.4:2000SEP08 | g4328020 | 3488 | 3775 |
| 66 | LG:1045521.4:2000SEP08 | 6616957H1 | 1254 | 1386 |
| 66 | LG:1045521.4:2000SEP08 | 5447708H2 | 3314 | 3558 |
| 66 | LG:1045521.4:2000SEP08 | 2115461H1 | 1425 | 1704 |
| 66 | LG:1045521.4:2000SEP08 | g1947654 | 1424 | 1631 |
| 66 | LG:1045521.4:2000SEP08 | 3526153H1 | 1444 | 1718 |
| 66 | LG:1045521.4:2000SEP08 | 2758729F6 | 1418 | 1919 |
| 66 | LG:1045521.4:2000SEP08 | 3151868H1 | 1417 | 1703 |
| 66 | LG:1045521.4:2000SEP08 | 3151862T6 | 1417 | 1880 |
| 66 | LG:1045521.4:2000SEP08 | 7705621J1 | 2286 | 2850 |
| 66 | LG:1045521.4:2000SEP08 | 6549421H1 | 1277 | 1806 |
| 66 | LG:1045521.4:2000SEP08 | 6549423H1 | 1277 | 1587 |
| 66 | LG:1045521.4:2000SEP08 | 917768H1 | 1289 | 1522 |
| 66 | LG:1045521.4:2000SEP08 | 915656H1 | 1289 | 1584 |
| 66 | LG:1045521.4:2000SEP08 | 1688229T6 | 1310 | 1882 |
| 66 | LG:1045521.4:2000SEP08 | 2758729H1 | 1324 | 1502 |
| 66 | LG:1045521.4:2000SEP08 | 2758729R6 | 1324 | 1851 |
| 66 | LG:1045521.4:2000SEP08 | 5813069T8 | 1334 | 1842 |
| 66 | LG:1045521.4:2000SEP08 | 945106H1 | 1329 | 1605 |
| 66 | LG:1045521.4:2000SEP08 | 1686019H1 | 1331 | 1520 |
| 66 | LG:1045521.4:2000SEP08 | 1686019F6 | 1331 | 1850 |
| 66 | LG:1045521.4:2000SEP08 | 5104890W | 1334 | 1560 |
| 66 | LG:1045521.4:2000SEP08 | 5104512H1 | 1336 | 1575 |
| 66 | LG:1045521.4:2000SEP08 | 6500521H1 | 1360 | 1913 |
| 66 | LG:1045521.4:2000SEP08 | 601501H1 | 1382 | 1634 |
| 66 | LG:1045521.4:2000SEP08 | 3151862R6 | 1417 | 1919 |
| 66 | LG:1045521.4:2000SEP08 | 2510294H1 | 934 | 1252 |
| 66 | LG:1045521.4:2000SEP08 | 2510294F6 | 937 | 1482 |
| 66 | LG:1045521.4:2000SEP08 | 6977627H1 | 964 | 1515 |
| 66 | LG:1045521.4:2000SEP08 | 3297985H1 | 973 | 1220 |
| 66 | LG:1045521.4:2000SEP08 | g1964154 | 1107 | 1527 |
| 66 | LG:1045521.4:2000SEP08 | 3951209H1 | 1127 | 1375 |
| 66 | LG:1045521.4:2000SEP08 | 7279066H1 | 1131 | 1540 |
| 66 | LG:1045521.4:2000SEP08 | 6839180H1 | 1147 | 1699 |
| 66 | LG:1045521.4:2000SEP08 | 5542001H1 | 1154 | 1374 |
| 66 | LG:1045521.4:2000SEP08 | 4892761H1 | 1181 | 1459 |
| 66 | LG:1045521.4:2000SEP08 | g2159379 | 1189 | 1613 |
| 66 | LG:1045521.4:2000SEP08 | 5387974H1 | 1192 | 1461 |
| 66 | LG:1045521.4:2000SEP08 | 3069606H1 | 1208 | 1500 |
| 66 | LG:1045521.4:2000SEP08 | 3501320H1 | 1214 | 1509 |
| 66 | LG:1045521.4:2000SEP08 | 5960233H1 | 1223 | 1761 |
| 66 | LG:1045521.4:2000SEP08 | 5817458H1 | 1231 | 1513 |
| 66 | LG:1045521.4:2000SEP08 | 5821151H1 | 1231 | 1513 |
| 66 | LG:1045521.4:2000SEP08 | 6315173H1 | 1231 | 1819 |
| 66 | LG:1045521.4:2000SEP08 | 985023R1 | 1248 | 1697 |
| 66 | LG:1045521.4:2000SEP08 | 985023H1 | 1248 | 1524 |
| 66 | LG:1045521.4:2000SEP08 | 6270034H2 | 1272 | 1798 |
| 66 | LG:1045521.4:2000SEP08 | 2643406H1 | 330 | 583 |
| 66 | LG:1045521.4:2000SEP08 | 7256990H1 | 431 | 803 |
| 66 | LG:1045521.4:2000SEP08 | 5877287H1 | 498 | 743 |
| 66 | LG:1045521.4:2000SEP08 | 2112746H1 | 499 | 749 |
| 66 | LG:1045521.4:2000SEP08 | 5072105H1 | 543 | 816 |
| 66 | LG:1045521.4:2000SEP08 | 1493714H1 | 599 | 814 |
| 66 | LG:1045521.4:2000SEP08 | 5897630H1 | 620 | 928 |
| 66 | LG:1045521.4:2000SEP08 | 6041048H1 | 625 | 1192 |
| 66 | LG:1045521.4:2000SEP08 | g707605 | 636 | 884 |
| 66 | LG:1045521.4:2000SEP08 | 7280773H1 | 638 | 1185 |
| 66 | LG:1045521.4:2000SEP08 | g1815503 | 641 | 896 |
| 66 | LG:1045521.4:2000SEP08 | 5598229H1 | 677 | 926 |
| 66 | LG:1045521.4:2000SEP08 | 2706109F6 | 683 | 1153 |
| 66 | LG:1045521.4:2000SEP08 | 2706109H1 | 683 | 978 |
| 66 | LG:1045521.4:2000SEP08 | 4308862H1 | 708 | 842 |
| 66 | LG:1045521.4:2000SEP08 | g1189689 | 727 | 1134 |
| 66 | LG:1045521.4:2000SEP08 | 3268156H1 | 746 | 981 |
| 66 | LG:1045521.4:2000SEP08 | g1959888 | 747 | 1207 |
| 66 | LG:1045521.4:2000SEP08 | 6913104J1 | 765 | 1279 |
| 66 | LG:1045521.4:2000SEP08 | g1692292 | 767 | 1116 |
| 66 | LG:1045521.4:2000SEP08 | 7584006H1 | 858 | 941 |
| 66 | LG:1045521.4:2000SEP08 | 7280450H1 | 867 | 1393 |
| 66 | LG:1045521.4:2000SEP08 | 4557319H1 | 873 | 1128 |
| 66 | LG:1045521.4:2000SEP08 | 6315112H1 | 873 | 996 |
| 66 | LG:1045521.4:2000SEP08 | 7170747H1 | 875 | 972 |
| 66 | LG:1045521.4:2000SEP08 | 4127083H1 | 909 | 1138 |
| 66 | LG:1045521.4:2000SEP08 | g4243806 | 1 | 340 |
| 66 | LG:1045521.4:2000SEP08 | 7601257J1 | 1 | 256 |
| 66 | LG:1045521.4:2000SEP08 | g4244618 | 1 | 450 |
| 66 | LG:1045521.4:2000SEP08 | g4851668 | 1 | 475 |
| 66 | LG:1045521.4:2000SEP08 | g4111830 | 1 | 469 |
| 66 | LG:1045521.4:2000SEP08 | g898045 | 1 | 349 |
| 66 | LG:1045521.4:2000SEP08 | 6818820J1 | 1 | 232 |
| 66 | LG:1045521.4:2000SEP08 | 6764773J1 | 1 | 591 |
| 66 | LG:1045521.4:2000SEP08 | g5511183 | 1 | 476 |
| 66 | LG:1045521.4:2000SEP08 | g5590133 | 16 | 474 |
| 66 | LG:1045521.4:2000SEP08 | g4088559 | 33 | 452 |
| 66 | LG:1045521.4:2000SEP08 | g4300922 | 33 | 418 |
| 66 | LG:1045521.4:2000SEP08 | g898069 | 40 | 304 |
| 66 | LG:1045521.4:2000SEP08 | g810354 | 45 | 328 |
| 66 | LG:1045521.4:2000SEP08 | g2898466 | 48 | 455 |
| 66 | LG:1045521.4:2000SEP08 | g3050233 | 49 | 227 |
| 66 | LG:1045521.4:2000SEP08 | 5623371H1 | 71 | 387 |
| 66 | LG:1045521.4:2000SEP08 | 6863535H1 | 104 | 594 |
| 66 | LG:1045521.4:2000SEP08 6996631H1 | 111 | 438 | |
| 66 | LG:1045521.4:2000SEP08 | g1648497 | 139 | 498 |
| 66 | LG:1045521.4:2000SEP08 | g1668085 | 139 | 495 |
| 66 | LG:1045521.4:2000SEP08 | g774564 | 177 | 307 |
| 66 | LG:1045521.4:2000SEP08 | g810462 | 259 | 566 |
| 66 | LG:1045521.4:2000SEP08 | 7081930H1 | 287 | 837 |
| 66 | LG:1045521.4:2000SEP08 | 2643406F6 | 330 | 832 |
| 66 | LG:1045521.4:2000SEP08 | 5813069F8 | 1231 | 1851 |
| 66 | LG:1045521.4:2000SEP08 | 1551431H1 | 3304 | 3506 |
| 66 | LG:1045521.4:2000SEP08 | 1811620H1 | 3411 | 3589 |
| 66 | LG:1045521.4:2000SEP08 | 4722921H1 | 3449 | 3513 |
| 67 | LG:275876.1:2000SEP08 | 5502856R6 | 711 | 1190 |
| 67 | LG:275876.1:2000SEP08 | g6704557 | 1 | 400 |
| 67 | LG:275876.1:2000SEP08 | g3250183 | 33 | 488 |
| 67 | LG:275876.1:2000SEP08 | 4251835H1 | 91 | 305 |
| 67 | LG:275876.1:2000SEP08 | 6880661H1 | 263 | 857 |
| 68 | LG:475127.7:2000SEP08 | g1964734 | 1 | 363 |
| 68 | LG:475127.7:2000SEP08 | 7169164H1 | 1 | 501 |
| 68 | LG:475127.7:2000SEP08 | 3601433H1 | 160 | 458 |
| 69 | LG:157263.1:2000SEP08 | 7401256H1 | 1076 | 1531 |
| 69 | LG:157263.1:2000SEP08 | g4018570 | 221 | 444 |
| 69 | LG:157263.1:2000SEP08 | 7618286H1 | 1 | 565 |
| 69 | LG:157263.1:2000SEP08 | 6916646H1 | 406 | 716 |
| 69 | LG:157263.1:2000SEP08 | 7641384H1 | 475 | 826 |
| 69 | LG:157263.1:2000SEP08 | 7765238H1 | 569 | 1157 |
| 69 | LG:157263.1:2000SEP08 | 7618286J1 | 700 | 1286 |
| 69 | LG:157263.1:2000SEP08 | 7765238J1 | 929 | 1542 |
| 69 | LG:157263.1:2000SEP08 | 817477H1 | 958 | 1222 |
| 70 | LG:247382.7:2000SEP08 | 2989208H1 | 1 | 192 |
| 70 | LG:247382.7:2000SEP08 | 7600764J1 | 1 | 498 |
| 70 | LG:247382.7:2000SEP08 | 7600764H1 | 16 | 515 |
| 70 | LG:247382.7:2000SEP08 | 2071263H1 | 1108 | 1372 |
| 70 | LG:247382.7:2000SEP08 | 2852080H1 | 1140 | 1356 |
| 70 | LG:247382.7:2000SEP08 | 7703934H1 | 1154 | 1680 |
| 70 | LG:247382.7:2000SEP08 | 3532904H1 | 1196 | 1480 |
| 70 | LG:247382.7:2000SEP08 | 4305547H1 | 1235 | 1545 |
| 70 | LG:247382.7:2000SEP08 | 4640181H1 | 1462 | 1671 |
| 70 | LG:247382.7:2000SEP08 | 7697061H1 | 495 | 1087 |
| 70 | LG:247382.7:2000SEP08 | 2686388H1 | 515 | 755 |
| 70 | LG:247382.7:2000SEP08 | 010297H1 | 533 | 800 |
| 70 | LG:247382.7:2000SEP08 | 010377H1 | 533 | 816 |
| 70 | LG:247382.7:2000SEP08 | 010299H1 | 535 | 796 |
| 70 | LG:247382.7:2000SEP08 | 013850H1 | 550 | 837 |
| 70 | LG:247382.7:2000SEP08 | 013958H1 | 550 | 831 |
| 70 | LG:247382.7:2000SEP08 | g5540183 | 605 | 952 |
| 70 | LG:247382.7:2000SEP08 | 5291818H1 | 670 | 929 |
| 70 | LG:247382.7:2000SEP08 | 7703934J1 | 869 | 1414 |
| 70 | LG:247382.7:2000SEP08 | 2503245H1 929 | 1156 | |
| 70 | LG:247382.7:2000SEP08 | 3233065H1 | 87 | 179 |
| 70 | LG:247382.7:2000SEP08 | 5090313F6 | 326 | 703 |
| 70 | LG:247382.7:2000SEP08 | 5090313H1 | 327 | 559 |
| 70 | LG:247382.7:2000SEP08 | 5047038F6 | 362 | 983 |
| 70 | LG:247382.7:2000SEP08 | 5047038H1 | 364 | 646 |
| 70 | LG:247382.7:2000SEP08 | 701 3633H1 | 482 | 764 |
| 71 | LG:197367.5:2000SEP08 | 6032665H1 | 1 | 569 |
| 72 | LG:218090.5:2000SEP08 | g4970262 | 554 | 899 |
| 72 | LG:218090.5:2000SEP08 | 7619489H1 | 1 | 594 |
| 72 | LG:218090.5:2000SEP08 | 5218316H1 | 34 | 271 |
| 72 | LG:218090.5:2000SEP08 | g5838654 | 37 | 288 |
| 73 | LG:216612.4:2000SEP08 | 6927865H1 | 598 | 1049 |
| 73 | LG:216612.4:2000SEP08 | 6765038H1 | 1 | 356 |
| 73 | LG:216612.4:2000SEP08 | 6823189H1 | 1 | 515 |
| 73 | LG:216612.4:2000SEP08 | 6823189J1 | 268 | 821 |
| 73 | LG:216612.4:2000SEP08 | 4699496F7 | 688 | 1261 |
| 73 | LG:216612.4:2000SEP08 | 4699496H1 | 688 | 891 |
| 73 | LG:216612.4:2000SEP08 | 6126235F6 | 1004 | 1666 |
| 73 | LG:216612.4:2000SEP08 | 6118801H1 | 1004 | 1129 |
| 73 | LG:216612.4:2000SEP08 | g1012738 | 705 | 946 |
| 73 | LG:216612.4:2000SEP08 | 7719209J1 | 752 | 1298 |
| 73 | LG:216612.4:2000SEP08 | 7744047H1 | 782 | 1296 |
| 73 | LG:216612.4:2000SEP08 | g794554 | 2469 | 2740 |
| 73 | LG:216612.4:2000SEP08 | 5905071F8 | 2017 | 2563 |
| 73 | LG:216612.4:2000SEP08 | 5905071F6 | 2022 | 2389 |
| 73 | LG:216612.4:2000SEP08 | 5905071H1 | 2022 | 2292 |
| 73 | LG:216612.4:2000SEP08 | 5905071T9 | 2037 | 2624 |
| 73 | LG:216612.4:2000SEP08 | 7744047J1 | 2120 | 2706 |
| 73 | LG:216612.4:2000SEP08 | g562707 | 2447 | 2728 |
| 73 | LG:216612.4:2000SEP08 | g764767 | 1368 | 1693 |
| 73 | LG:216612.4:2000SEP08 | 7719209H1 | 1382 | 1940 |
| 73 | LG:216612.4:2000SEP08 | 6765038J1 | 1574 | 2159 |
| 73 | LG:216612.4:2000SEP08 | g1012737 | 1627 | 1867 |
| 73 | LG:216612.4:2000SEP08 | 5905171H1 | 2013 | 2278 |
| 73 | LG:216612.4:2000SEP08 | 6126235H1 | 1004 | 1556 |
| 73 | LG:216612.4:2000SEP08 | 4699496T7 | 1179 | 1651 |
| 73 | LG:216612.4:2000SEP08 | g572752 | 1367 | 1734 |
| 74 | LG:197614.1:2000SEP08 | 1287527H1 | 3708 | 3958 |
| 74 | LG:197614.1:2000SEP08 | 2286805H1 | 3713 | 3944 |
| 74 | LG:197614.1:2000SEP08 | g1165770 | 3724 | 3845 |
| 74 | LG:197614.1:2000SEP08 | 2169536H1 | 3730 | 3980 |
| 74 | LG:197614.1:2000SEP08 | 3452306H1 | 3730 | 3894 |
| 74 | LG:197614.1:2000SEP08 | 628239H1 | 3750 | 4013 |
| 74 | LG:197614.1:2000SEP08 | 5306905H1 | 3778 | 3895 |
| 74 | LG:197614.1:2000SEP08 | 5757045H1 | 3806 | 4001 |
| 74 | LG:197614.1:2000SEP08 | 1287527F1 | 3708 | 4248 |
| 74 | LG:197614.1:2000SEP08 | 5898662H1 | 3622 | 3910 |
| 74 | LG:197614.1:2000SEP08 | 5895501H1 | 3623 | 3920 |
| 74 | LG:197614.1:2000SEP08 | g2140565 | 3623 | 3950 |
| 74 | LG:197614.1:2000SEP08 | 5730757H1 | 3622 | 3823 |
| 74 | LG:197614.1:2000SEP08 | 1544877H1 | 3671 | 3870 |
| 74 | LG:197614.1:2000SEP08 | 5271113H1 | 3701 | 3950 |
| 74 | LG:197614.1:2000SEP08 | 6085421H1 | 3704 | 4135 |
| 74 | LG:197614.1:2000SEP08 | g1933869 | 3703 | 3944 |
| 74 | LG:197614.1:2000SEP08 | g2899129 | 2999 | 3411 |
| 74 | LG:197614.1:2000SEP08 | g5425670 | 3002 | 3466 |
| 74 | LG:197614.1:2000SEP08 | 3449642H1 | 3016 | 3248 |
| 74 | LG:197614.1:2000SEP08 | g5812471 | 54 | 465 |
| 74 | LG:197614.1:2000SEP08 | 5914544H1 | 767 | 939 |
| 74 | LG:197614.1:2000SEP08 | 3105863H1 | 146 | 423 |
| 74 | LG:197614.1:2000SEP08 | 3270954H1 | 150 | 283 |
| 74 | LG:197614.1:2000SEP08 | g4899736 | 204 | 582 |
| 74 | LG:197614.1:2000SEP08 | g4153549 | 211 | 637 |
| 74 | LG:197614.1:2000SEP08 | g5849305 | 214 | 637 |
| 74 | LG:197614.1:2000SEP08 | g1815090 | 228 | 641 |
| 74 | LG:197614.1:2000SEP08 | g4899744 | 250 | 581 |
| 74 | LG:197614.1:2000SEP08 | g1624602 | 340 | 638 |
| 74 | LG:197614.1:2000SEP08 | g734108 | 416 | 607 |
| 74 | LG:197614.1:2000SEP08 | 6608771H1 | 573 | 1078 |
| 74 | LG:197614.1:2000SEP08 | 7467077H1 | 692 | 1231 |
| 74 | LG:197614.1:2000SEP08 | g2620587 | 4369 | 4614 |
| 74 | LG:197614.1:2000SEP08 | g777201 | 4372 | 4614 |
| 74 | LG:197614.1:2000SEP08 | g4523723 | 4378 | 4613 |
| 74 | LG:197614.1:2000SEP08 | 5161106H1 | 4398 | 4607 |
| 74 | LG:197614.1:2000SEP08 | 3690612H1 | 4464 | 4758 |
| 74 | LG:197614.1:2000SEP08 | 3702535H1 | 4476 | 4613 |
| 74 | LG:197614.1:2000SEP08 | 661314T6 | 4484 | 4570 |
| 74 | LG:197614.1:2000SEP08 | 1929304H1 | 4529 | 4768 |
| 74 | LG:197614.1:2000SEP08 | 1929304F6 | 4529 | 4748 |
| 74 | LG:197614.1:2000SEP08 | 3010805H1 | 4578 | 4864 |
| 74 | LG:197614.1:2000SEP08 | g6661262 | 1 | 472 |
| 74 | LG:197614.1:2000SEP08 | g3401176 | 1 | 445 |
| 74 | LG:197614.1:2000SEP08 | g5637467 | 1 | 499 |
| 74 | LG:197614.1:2000SEP08 | g5755549 | 1 | 448 |
| 74 | LG:197614.1:2000SEP08 | g1614502 | 1 | 480 |
| 74 | LG:197614.1:2000SEP08 | 6763231J1 | 11 | 86 |
| 74 | LG:197614.1:2000SEP08 | g4305963 | 23 | 478 |
| 74 | LG:197614.1:2000SEP08 | g3232676 | 24 | 468 |
| 74 | LG:197614.1:2000SEP08 | g6713108 | 33 | 494 |
| 74 | LG:197614.1:2000SEP08 | g4888325 | 42 | 466 |
| 74 | LG:197614.1:2000SEP08 | g2910679 | 34 | 458 |
| 74 | LG:197614.1:2000SEP08 | g5635762 | 49 | 399 |
| 74 | LG:197614.1:2000SEP08 | 564653H1 | 4227 | 4440 |
| 74 | LG:197614.1:2000SEP08 | 4827983H1 | 4317 | 4583 |
| 74 | LG:197614.1:2000SEP08 | g2877107 | 4324 | 4617 |
| 74 | LG:197614.1:2000SEP08 | 2504732H1 | 4228 | 4450 |
| 74 | LG:197614.1:2000SEP08 | 3525080H1 | 4231 | 4496 |
| 74 | LG:197614.1:2000SEP08 | 2296610H1 | 4241 | 4501 |
| 74 | LG:197614.1:2000SEP08 | 704337H1 | 4258 | 4476 |
| 74 | LG:197614.1:2000SEP08 | 6736875H1 | 4259 | 4602 |
| 74 | LG:197614.1:2000SEP08 | 1638150T6 | 4295 | 4578 |
| 74 | LG:197614.1:2000SEP08 | 4615882H1 | 4316 | 4570 |
| 74 | LG:197614.1:2000SEP08 | 1358968H1 | 4365 | 4605 |
| 74 | LG:197614.1:2000SEP08 | 1916409H1 | 3605 | 3856 |
| 74 | LG:197614.1:2000SEP08 | 180009R6 | 3093 | 3516 |
| 74 | LG:197614.1:2000SEP08 | 180009H1 | 3093 | 3275 |
| 74 | LG:197614.1:2000SEP08 | g1490176 | 3095 | 3467 |
| 74 | LG:197614.1:2000SEP08 | 3117564H1 | 3095 | 3392 |
| 74 | LG:197614.1:2000SEP08 | g2902729 | 3096 | 3466 |
| 74 | LG:197614.1:2000SEP08 | 4523772H1 | 3108 | 3352 |
| 74 | LG:197614.1:2000SEP08 | g3841726 | 3126 | 3467 |
| 74 | LG:197614.1:2000SEP08 | g1492261 | 3133 | 3467 |
| 74 | LG:197614.1:2000SEP08 | 5334175H1 | 3146 | 3312 |
| 74 | LG:197614.1:2000SEP08 | 463918H1 | 3146 | 3386 |
| 74 | LG:197614.1:2000SEP08 | g1886770 | 3148 | 3439 |
| 74 | LG:197614.1:2000SEP08 | 1453577F6 | 3179 | 3472 |
| 74 | LG:197614.1:2000SEP08 | 1452163H1 | 3179 | 3432 |
| 74 | LG:197614.1:2000SEP08 | 1452163F1 | 3179 | 3649 |
| 74 | LG:197614.1:2000SEP08 | 1453577H1 | 3179 | 3432 |
| 74 | LG:197614.1:2000SEP08 | g1635322 | 3203 | 3378 |
| 74 | LG:197614.1:2000SEP08 | 4883761H1 | 3218 | 3425 |
| 74 | LG:197614.1:2000SEP08 | g4534089 | 3221 | 3467 |
| 74 | LG:197614.1:2000SEP08 | 5873648H1 | 3289 | 3537 |
| 74 | LG:197614.1:2000SEP08 | 7132968H1 | 3304 | 3749 |
| 74 | LG:197614.1:2000SEP08 | 3516403H1 | 3312 | 3559 |
| 74 | LG:197614.1:2000SEP08 | 4261 15T6 | 3327 | 3789 |
| 74 | LG:197614.1:2000SEP08 | 5664838H1 | 3325 | 3613 |
| 74 | LG:197614.1:20003EP08 | 3514295H1 | 3364 | 3619 |
| 74 | LG:197614.1:2000SEP08 | 2568145H1 | 3378 | 3627 |
| 74 | LG:197614.1:2000SEP08 | 2569762H1 | 3378 | 3633 |
| 74 | LG:197614.1:20003EP08 | 4898290H1 | 3614 | 3884 |
| 74 | LG:197614.1:2000SEP08 | g2047002 | 3401 | 3794 |
| 74 | LG:197614.1:2000SEP08 | g2047020 | 3401 | 3744 |
| 74 | LG:197614.1:2000SEP08 | 7137103H1 | 3618 | 3749 |
| 74 | LG:197614.1:2000SEP08 | 6608771T1 | 3442 | 3878 |
| 74 | LG:197614.1:2000SEP08 | 6875871H1 | 3442 | 3973 |
| 74 | LG:197614.1:2000SEP08 | 060722H1 | 3479 | 3591 |
| 74 | LG:197614.1:2000SEP08 | 5833944H1 | 3484 | 3743 |
| 74 | LG:197614.1:2000SEP08 | g2838778 | 3504 | 3964 |
| 74 | LG:197614.1:2000SEP08 | 1668733T6 | 3509 | 3907 |
| 74 | LG:197614.1:2000SEP08 | g4285920 | 3515 | 3949 |
| 74 | LG:197614.1:2000SEP08 | 1668733F6 | 3516 | 3890 |
| 74 | LG:197614.1:2000SEP08 | 1668733H1 | 3516 | 3739 |
| 74 | LG:197614.1:2000SEP08 | 5301951H1 | 3528 | 3739 |
| 74 | LG:197614.1:2000SEP08 | g6946407 | 3543 | 3948 |
| 74 | LG:197614.1:2000SEP08 | g2140557 | 3549 | 3950 |
| 74 | LG:197614.1:2000SEP08 | 871967R1 | 3572 | 3994 |
| 74 | LG:197614.1:2000SEP08 | 871967H1 | 3573 | 3812 |
| 74 | LG:197614.1:2000SEP08 | 3510984H1 | 3603 | 3826 |
| 74 | LG:197614.1:2000SEP08 | 2041903H1 | 3602 | 3769 |
| 74 | LG:197614.1:2000SEP08 | 5904560H1 | 3622 | 3920 |
| 74 | LG:197614.1:2000SEP08 | g5444559 | 4204 | 4615 |
| 74 | LG:197614.1:2000SEP08 | g2820548 | 4204 | 4613 |
| 74 | LG:197614.1:2000SEP08 | g4896092 | 4204 | 4606 |
| 74 | LG:197614.1:2000SEP08 | g5447131 | 4204 | 4613 |
| 74 | LG:197614.1:2000SEP08 | 1638150H1 | 4204 | 4351 |
| 74 | LG:197614.1:2000SEP08 | 701181H1 | 4204 | 4370 |
| 74 | LG:197614.1:2000SEP08 | g2563289 | 4206 | 4577 |
| 74 | LG:197614.1:2000SEP08 | 2437436H1 | 4221 | 4443 |
| 74 | LG:197614.1:2000SEP08 | 1638150F6 | 4204 | 4545 |
| 74 | LG:197614.1:2000SEP08 | 426115H1 | 2948 | 3152 |
| 74 | LG:197614.1:2000SEP08 | g5528772 | 2988 | 3466 |
| 74 | LG:197614.1:2000SEP08 | 1581709H1 | 4096 | 4291 |
| 74 | LG:197614.1:2000SEP08 | 1581803H1 | 4096 | 4307 |
| 74 | LG:197614.1:2000SEP08 | 5652453H1 | 4108 | 4614 |
| 74 | LG:197614.1:2000SEP08 | 5732485T6 | 4093 | 4566 |
| 74 | LG:197614.1:2000SEP08 | 1581784H1 | 4096 | 4291 |
| 74 | LG:197614.1:2000SEP08 | g4739964 | 4187 | 4609 |
| 74 | LG:197614.1:2000SEP08 | g3898531 | 4197 | 4614 |
| 74 | LG:197614.1:2000SEP08 | g4243541 | 4197 | 4614 |
| 74 | LG:197614.1:2000SEP08 | g6397736 | 4203 | 4615 |
| 74 | LG:197614.1:2000SEP08 | 5809075H2 | 2712 | 2950 |
| 74 | LG:197614.1:2000SEP08 | 660019H1 | 2845 | 3116 |
| 74 | LG:197614.1:2000SEP08 | 7070142H1 | 2718 | 3259 |
| 74 | LG:197614.1:2000SEP08 | g4896323 | 2733 | 3113 |
| 74 | LG:197614.1:2000SEP08 | 661314H1 | 2845 | 3130 |
| 74 | LG:197614.1:2000SEP08 | 661314R6 | 2845 | 3187 |
| 74 | LG:197614.1:2000SEP08 | g1933868 | 2740 | 3080 |
| 74 | LG:197614.1:2000SEP08 | 6025513H1 | 2853 | 3151 |
| 74 | LG:197614.1:2000SEP08 | 6931975H1 | 2747 | 3260 |
| 74 | LG:197614.1:2000SEP08 | g1886879 | 2748 | 3105 |
| 74 | LG:197614.1:2000SEP08 | 3570587H1 | 2804 | 3083 |
| 74 | LG:197614.1:2000SEP08 | 6197889H1 | 2815 | 3281 |
| 74 | LG:197614.1:2000SEP08 | 6202948H1 | 2817 | 3392 |
| 74 | LG:197614.1:2000SEP08 | g1491312 | 2821 | 3260 |
| 74 | LG:197614.1:2000SEP08 | 7664971H1 | 2823 | 3356 |
| 74 | LG:197614.1:2000SEP08 | 660648H1 | 2845 | 3118 |
| 74 | LG:197614.1:2000SEP08 | g1989594 | 2864 | 3024 |
| 74 | LG:197614.1:2000SEP08 | 378504H1 | 2428 | 2614 |
| 74 | LG:197614.1:2000SEP08 | 378504R6 | 2428 | 2929 |
| 74 | LG:197614.1:2000SEP08 | 5834173H1 | 2474 | 2744 |
| 74 | LG:197614.1:2000SEP08 | 5391905H1 | 2515 | 2792 |
| 74 | LG:197614.1:2000SEP08 | 2534074H1 | 2628 | 2875 |
| 74 | LG:197614.1:2000SEP08 | 6951984H1 | 2643 | 3252 |
| 74 | LG:197614.1:2000SEP08 | 7621265H1 | 2095 | 2621 |
| 74 | LG:197614.1:2000SEP08 | 5729256H1 | 2254 | 2747 |
| 74 | LG:197614.1:2000SEP08 | 7439141H1 | 2355 | 2829 |
| 74 | LG:197614.1:2000SEP08 | g1747961 | 2280 | 2586 |
| 74 | LG:197614.1:2000SEP08 | 5349468H1 | 2351 | 2584 |
| 74 | LG:197614.1:2000SEP08 | 6763231H1 | 2374 | 2905 |
| 74 | LG:197614.1:2000SEP08 | 7650036H2 | 2417 | 2975 |
| 74 | LG:197614.1:2000SEP08 | 7156980H1 | 938 | 1507 |
| 74 | LG:197614.1:2000SEP08 | 7663421H1 | 1134 | 1649 |
| 74 | LG:197614.1:2000SEP08 | 7650036J2 | 1157 | 1772 |
| 74 | LG:197614.1:2000SEP08 | 7359954H1 | 1252 | 1784 |
| 74 | LG:197614.1:2000SEP08 | 7621265J1 | 1262 | 1833 |
| 74 | LG:197614.1:2000SEP08 | 5732485H1 | 1126 | 1318 |
| 74 | LG:197614.1:2000SEP08 | 7080048H1 | 1640 | 2223 |
| 74 | LG:197614.1:2000SEP08 | 6485720H1 | 1880 | 2304 |
| 74 | LG:197614.1:2000SEP08 | 6485720F9 | 1926 | 2075 |
| 74 | LG:197614.1:2000SEP08 | 081459H1 | 1973 | 2232 |
| 74 | LG:197614.1:2000SEP08 | 7663421J1 | 2044 | 2608 |
| 74 | LG:197614.1:2000SEP08 | g3423345 | 3018 | 3467 |
| 74 | LG:197614.1:2000SEP08 | g5590054 | 3020 | 3467 |
| 74 | LG:197614.1:2000SEP08 | g4111681 | 3027 | 3467 |
| 74 | LG:197614.1:2000SEP08 | 2617018H2 | 3029 | 3265 |
| 74 | LG:197614.1:2000SEP08 | 3815347H1 | 3033 | 3315 |
| 74 | LG:197614.1:2000SEP08 | g777251 | 3046 | 3330 |
| 74 | LG:197614.1:2000SEP08 | g2817792 | 3086 | 3475 |
| 74 | LG:197614.1:2000SEP08 | 4897664H1 | 3826 | 3991 |
| 74 | LG:197614.1:2000SEP08 | 4896259H1 | 3849 | 4001 |
| 74 | LG:197614.1:2000SEP08 | g1212198 | 3850 | 4178 |
| 74 | LG:197614.1:2000SEP08 | g1748459 | 3880 | 3967 |
| 74 | LG:197614.1:2000SEP08 | 5063386H1 | 3886 | 4001 |
| 74 | LG:197614.1:2000SEP08 | 1491069H1 | 3900 | 4105 |
| 74 | LG:197614.1:2000SEP08 | 1491069F6 | 3900 | 4001 |
| 74 | LG:197614.1:2000SEP08 | g896855 | 4022 | 4245 |
| 74 | LG:197614.1:2000SEP08 | g4901112 | 4022 | 4378 |
| 74 | LG:197614.1:2000SEP08 | g4990162 | 4025 | 4297 |
| 74 | LG:197614.1:2000SEP08 | 96699628 | 4025 | 4483 |
| 74 | LG:197614.1:2000SEP08 | 180009T6 | 4072 | 4569 |
| 74 | LG:197614.1:2000SEP08 | 1491069T6 | 4075 | 4576 |
| 74 | LG:197614.1:2000SEP08 | 6208837H1 | 4088 | 4388 |
| 74 | LG:197614.1:2000SEP08 | 7355849H1 | 4097 | 4587 |
| 74 | LG:197614.1:2000SEP08 | 2588654H1 | 2874 | 3132 |
| 74 | LG:197614.1:2000SEP08 | 4261 15R6 | 2948 | 3453 |
| 74 | LG:197614.1:2000SEP08 | g1492260 | 2879 | 3120 |
| 74 | LG:197614.1:2000SEP08 | 3873481H1 | 2937 | 3214 |
| 74 | LG:197614.1:2000SEP08 | 425031H1 | 2948 | 3210 |
| 75 | LG:378428.1:2000SEP08 | 6910888F8 | 560 | 708 |
| 75 | LG:378428.1:2000SEP08 | 7379444H1 | 36 | 599 |
| 75 | LG:378428.1:2000SEP08 | 6816543H1 | 185 | 640 |
| 75 | LG:378428.1:2000SEP08 | 6816543F8 | 285 | 641 |
| 75 | LG:378428.1:2000SEP08 | 7429391H1 | 439 | 956 |
| 75 | LG:378428.1:2000SEP08 | 6816543R8 | 1 | 644 |
| 75 | LG:378428.1:2000SEP08 | 7379418H1 | 36 | 609 |
| 75 | LG:378428.1:2000SEP08 | 6910888J1 | 768 | 1376 |
| 75 | LG:378428.1:2000SEP08 | 6910888R8 | 773 | 1449 |
| 75 | LG:378428.1:2000SEP08 | 6044422H1 | 726 | 1023 |
| 75 | LG:378428.1:2000SEP08 | 6044422F8 | 725 | 1341 |
| 75 | LG:378428.1:2000SEP08 | 6755205H 1 | 598 | 1201 |
| 75 | LG:378428.1:2000SEP08 | 54971 13R8 | 1551 | 2088 |
| 75 | LG:378428.1:2000SEP08 | 54971 13R9 | 1559 | 2088 |
| 75 | LG:378428.1:2000SEP08 | 5497113H1 | 1559 | 1823 |
| 75 | LG:378428.1:2000SEP08 | 6044422R8 | 1304 | 1879 |
| 75 | LG:378428.1:2000SEP08 | 4401305H1 | 1232 | 1481 |
| 75 | LG:378428.1:2000SEP08 | 6044422J1 | 1416 | 1879 |
| 75 | LG:378428.1:2000SEP08 | 6910888H1 | 1537 | 2074 |
| 75 | LG:378428.1:2000SEP08 | 4401305F6 | 1232 | 1757 |
| 75 | LG:378428.1:2000SEP08 | 5045827F6 | 1029 | 1501 |
| 75 | LG:378428.1:2000SEP08 | 5045827H1 | 1029 | 1306 |
| 75 | LG:378428.1:2000SEP08 | 7004756H1 | 1060 | 1494 |
| 75 | LG:378428.1:2000SEP08 | 7010551H1 | 1639 | 2000 |
| 75 | LG:378428.1:2000SEP08 | 5497113F9 | 1565 | 2088 |
| 75 | LG:378428.1:2000SEP08 | g2789298 | 2012 | 2404 |
| 75 | LG:378428.1:2000SEP08 | 4401305T6 | 2011 | 2370 |
| 75 | LG:378428.1:2000SEP08 | g2006832 | 2112 | 2414 |
| 75 | LG:378428.1:2000SEP08 | g6713687 | 1969 | 2395 |
| 75 | LG:378428.1:2000SEP08 | g2912647 | 1982 | 2398 |
| 76 | LG:286639.1:2000SEP08 | 5508394F6 | 681 | 1107 |
| 76 | LG:286639.1:2000SEP08 | 5508394H1 | 681 | 881 |
| 76 | LG:286639.1:2000SEP08 | 7687060J1 | 370 | 955 |
| 76 | LG:286639.1:2000SEP08 | 3217093F6 | 740 | 1174 |
| 76 | LG:286639.1:2000SEP08 | 3217093H1 | 740 | 1012 |
| 76 | LG:286639.1:2000SEP08 | g2025699 | 726 | 1059 |
| 76 | LG:286639.1:2000SEP08 | 301366H1 | 1211 | 1426 |
| 76 | LG:286639.1:2000SEP08 | 301366T6 | 1244 | 1768 |
| 76 | LG:286639.1:2000SEP08 | 3217093T6 | 1309 | 1844 |
| 76 | LG:286639.1:2000SEP08 | 5832442H1 | 1196 | 1448 |
| 76 | LG:286639.1:2000SEP08 | 301366R6 | 1211 | 1672 |
| 76 | LG:286639.1:2000SEP08 | 5507629H1 | 1181 | 1464 |
| 76 | LG:286639.1:2000SEP08 | 7687060H1 | 1024 | 1612 |
| 76 | LG:286639.1:20003EP08 | 4919304F6 | 1115 | 1523 |
| 76 | LG:286639.1:2000SEP08 | g3933409 | 1495 | 1975 |
| 76 | LG:286639.1:2000SEP08 | 4919304H1 | 1392 | 1523 |
| 76 | LG:286639.1:2000SEP08 | g2057022 | 1460 | 1800 |
| 76 | LG:286639.1:2000SEP08 | 6178302H1 | 1387 | 1560 |
| 76 | LG:286639.1:2000SEP08 | 4249162F6 | 1965 | 2347 |
| 76 | LG:286639.1:2000SEP08 | g4739468 | 1906 | 2281 |
| 76 | LG:286639.1:2000SEP08 | g5812196 | 1867 | 1966 |
| 76 | LG:286639.1:2000SEP08 | g5397776 | 1881 | 2281 |
| 76 | LG:286639.1:2000SEP08 | g4739487 | 1904 | 2281 |
| 76 | LG:286639.1:2000SEP08 | g3446647 | 1519 | 1975 |
| 76 | LG:286639.1:2000SEP08 | 5508394R6 | 1790 | 2257 |
| 76 | LG:286639.1:2000SEP08 | 4249162H1 | 1965 | 2115 |
| 76 | LG:286639.1:2000SEP08 | g3109536 | 2091 | 2281 |
| 76 | LG:286639.1:2000SEP08 | 95446112 | 2092 | 2281 |
| 76 | LG:286639.1:2000SEP08 | g6569543 | 1 466 | |
| 76 | LG:286639.1:2000SEP08 | 4138803H1 | 17 | 304 |
| 76 | LG:286639.1:2000SEP08 | 3595464H1344 | 623 | |
| 77 | LG:389870.1:2000SEP08 | 7065623H1 | 534 | 764 |
| 77 | LG:389870.1:2000SEP08 | 7191788H2 | 368 | 640 |
| 77 | LG:389870.1:2000SEP08 | 7354238H1 | 129 | 677 |
| 77 | LG:389870.1:2000SEP08 | g2740716 | 1 | 454 |
| 77 | LG:389870.1:2000SEP08 | g4389793 | 1 | 454 |
| 77 | LG:389870.1:2000SEP08 | 7459406H1 | 769 | 1222 |
| 77 | LG:389870.1:2000SEP08 | 4030167H1 | 620 | 850 |
| 77 | LG:389870.1:2000SEP08 | 7583301H1 | 710 | 1249 |
| 78 | LG:1387485.6:2000SEP08 | 1259442F6 | 727 | 1286 |
| 78 | LG:1387485.6:2000SEP08 | 575641H1 | 210 | 423 |
| 78 | LG:1387485.6:2000SEP08 | 2695041H1 | 441 | 744 |
| 78 | LG:1387485.6:2000SEP08 | 5394337H1 | 572 | 857 |
| 78 | LG:1387485.6:2000SEP08 | 4773576H1 | 617 | 849 |
| 78 | LG:1387485.6:2000SEP08 | 1722977H1 | 707 | 964 |
| 78 | LG:1387485.6:2000SEP08 | 1722977F6 | 707 | 1113 |
| 78 | LG:1387485.6:2000SEP08 | 1259442H1 | 726 | 966 |
| 78 | LG:1387485.6:2000SEP08 | 5004203H 1 | 894 | 1024 |
| 78 | LG:1387485.6:2000SEP08 | 4545090H1 | 897 | 1175 |
| 78 | LG:1387485.6:2000SEP08 | 29441 36F6 | 907 | 1283 |
| 78 | LG:1387485.6:2000SEP08 | 2944136H1 | 907 | 1198 |
| 78 | LG:1387485.6:2000SEP08 | 3495979H1 | 912 | 1194 |
| 78 | LG:1387485.6:2000SEP08 | g1202624 | 939 | 1381 |
| 78 | LG:1387485.6:2000SEP08 | g1012265 | 955 | 1249 |
| 78 | LG:1387485.6:2000SEP08 | g1042452 | 956 | 1258 |
| 78 | LG:1387485.6:2000SEP08 | g983177 | 962 | 1336 |
| 78 | LG:1387485.6:2000SEP08 | 3049691H1 | 963 | 1254 |
| 78 | LG:1387485.6:2000SEP08 | g1218110 | 1001 | 1269 |
| 78 | LG:1387485.6:2000SEP08 | 2652533H1 | 1016 | 1269 |
| 78 | LG:1387485.6:2000SEP08 | 2098947H1 | 1224 | 1456 |
| 78 | LG:1387485.6:2000SEP08 | 1309581H1 | 1328 | 1575 |
| 78 | LG:1387485.6:2000SEP08 | 1004812H1 | 1367 | 1552 |
| 78 | LG:1387485.6:2000SEP08 | 1568541H1 | 1392 | 1607 |
| 78 | LG:1387485.6:2000SEP08 | 2764023H1 | 1 | 259 |
| 78 | LG:1387485.6:2000SEP08 | 530901H1 | 2 | 134 |
| 78 | LG:1387485.6:2000SEP08 | 4049678H1 | 11 | 290 |
| 78 | LG:1387485.6:2000SEP08 | 3594169H1 | 11 | 279 |
| 78 | LG:1387485.6:2000SEP08 | 4330447H1 | 17 | 271 |
| 78 | LG:1387485.6:2000SEP08 | 4565262H 1 | 39 | 285 |
| 78 | LG:1387485.6:2000SEP08 | 6408733H 1 | 162 | 683 |
| 78 | LG:1387485.6:2000SEP08 | g1295497 | 175 | 615 |
| 78 | LG:1387485.6:2000SEP08 | 7039706H1 | 356 | 875 |
| 78 | LG:1387485.6:2000SEP08 | 986902H1 | 225 | 462 |
| 78 | LG:1387485.6:2000SEP08 | 7939046H1 | 446 | 921 |
| 78 | LG:1387485.6:2000SEP08 | 7270571H1 | 206 | 743 |
| 78 | LG:1387485.6:2000SEP08 | 1704717H1 | 255 | 425 |
| 78 | LG:1387485.6:2000SEP08 | 4723677H1 | 207 | 436 |
| 79 | LG:230151.1:2000SEP08 | g1760799 | 1489 | 1929 |
| 79 | LG:230151.1:2000SEP08 | 2834338F6 | 1524 | 2017 |
| 79 | LG:230151.1:2000SEP08 | 2588088F6 | 1290 | 1516 |
| 79 | LG:230151.1:2000SEP08 | 2588088H1 | 1290 | 1533 |
| 79 | LG:230151.1:2000SEP08 | 5562138H1 | 275 | 408 |
| 79 | LG:230151.1:2000SEP08 | 7387281H1 | 281 | 540 |
| 79 | LG:230151.1:2000SEP08 | g1678023 | 294 | 403 |
| 79 | LG:230151.1:2000SEP08 | 7625371H1 | 303 | 939 |
| 79 | LG:230151.1:2000SEP08 | g5037659 | 2811 | 2935 |
| 79 | LG:230151.1:2000SEP08 | g4094483 | 2814 | 2935 |
| 79 | LG:230151.1:2000SEP08 | 4420762H1 | 2222 | 2480 |
| 79 | LG:230151.1:2000SEP08 | 6868146H1 | 2238 | 2846 |
| 79 | LG:230151.1:2000SEP08 | 2692033H1 | 1 | 265 |
| 79 | LG:230151.1:2000SEP08 | 3345531H1 | 21 | 275 |
| 79 | LG:230151.1:2000SEP08 | g1990900 | 206 | 488 |
| 79 | LG:230151.1:2000SEP08 | 6057493H1 | 746 | 984 |
| 79 | LG:230151.1:2000SEP08 | 6057493F6 | 746 | 984 |
| 79 | LG:230151.1:2000SEP08 | 6057493F8 | 746 | 971 |
| 79 | LG:230151.1:2000SEP08 | 7625371J1 | 821 | 1444 |
| 79 | LG:230151.1:2000SEP08 | 198957R7 | 923 | 1449 |
| 79 | LG:230151.1:2000SEP08 | 198957H1 | 923 | 1108 |
| 79 | LG:230151.1:2000SEP08 | g1400401 | 1048 | 1444 |
| 79 | LG:230151.1:2000SEP08 | 4330089H1 | 194 | 378 |
| 79 | LG:230151.1:2000SEP08 | 7276116H2 | 85 | 666 |
| 79 | LG:230151.1:2000SEP08 | 5386485F7 | 114 | 726 |
| 79 | LG:230151.1:2000SEP08 | 5334046F6 | 553 | 998 |
| 79 | LG:230151.1:2000SEP08 | g1400455 | 625 | 896 |
| 79 | LG:230151.1:2000SEP08 | 6057485H1 | 746 | 984 |
| 79 | LG:230151.1:2000SEP08 | 2864522H1 | 2856 | 3146 |
| 79 | LG:230151.1:2000SEP08 | 7578937H1 | 74 | 626 |
| 79 | LG:230151.1:2000SEP08 | 2692033F6 | 1 | 503 |
| 79 | LG:230151.1:2000SEP08 | g3842498 | 2881 | 2935 |
| 79 | LG:230151.1:2000SEP08 | 2160735H1 | 2194 | 2444 |
| 79 | LG:230151.1:2000SEP08 | 2160580H1 | 2194 | 2448 |
| 79 | LG:230151.1:2000SEP08 | 2160735F6 | 2194 | 2588 |
| 79 | LG:230151.1:2000SEP08 | 9846162 | 2111 | 2428 |
| 79 | LG:230151.1:2000SEP08 | 6298190H1 | 2469 | 2740 |
| 79 | LG:230151.1:2000SEP08 | 1208532H1 | 2479 | 2545 |
| 79 | LG:230151.1:2000SEP08 | 1209024R6 | 2479 | 2912 |
| 79 | LG:230151.1:2000SEP08 | 1209024H1 | 2479 | 2707 |
| 79 | LG:230151.1:2000SEP08 | 4163123H1 | 2497 | 2793 |
| 79 | LG:230151.1:2000SEP08 | 2834338H1 | 1524 | 1795 |
| 79 | LG:230151.1:2000SEP08 | 4420745H1 | 2222 | 2481 |
| 79 | LG:230151.1:2000SEP08 | 1006278H1 | 1879 | 2136 |
| 79 | LG:230151.1:2000SEP08 | 6138222H1 | 2058 | 2353 |
| 79 | LG:230151.1:2000SEP08 | g694907 | 2556 | 2842 |
| 79 | LG:230151.1:2000SEP08 | 550609R1 | 2596 | 2935 |
| 79 | LG:230151.1:2000SEP08 | 7164680H1 | 1751 | 2245 |
| 79 | LG:230151.1:2000SEP08 | 1209024T6 | 2657 | 2912 |
| 79 | LG:230151.1:2000SEP08 | 19895717 | 2677 | 2912 |
| 79 | LG:230151.1:2000SEP08 | 6763572J1 | 2731 | 2935 |
| 79 | LG:230151.1:2000SEP08 | g1760656 | 2747 | 2935 |
| 79 | LG:230151.1:2000SEP08 | g3050231 | 2754 | 2935 |
| 79 | LG:230151.1:2000SEP08 | 7330830H2 | 2761 | 2912 |
| 79 | LG:230151.1:2000SEP08 | g2900238 | 2765 | 2935 |
| 79 | LG:230151.1:2000SEP08 | 92552192 | 2776 | 2899 |
| 79 | LG:230151.1:2000SEP08 | 3889714H1 | 2780 | 2933 |
| 79 | LG:230151.1:2000SEP08 | 5443914H1 | 2800 | 2912 |
| 79 | LG:230151.1:2000SEP08 | 550609R6 | 2596 | 2899 |
| 79 | LG:230151.1:2000SEP08 | 550609H1 | 2596 | 2866 |
| 79 | LG:230151.1:2000SEP08 | 550609F1 | 2605 | 2912 |
| 79 | LG:230151.1:2000SEP08 | 2588088T6 | 2627 | 2912 |
| 79 | LG:230151.1:2000SEP08 | 4552816H1 | 2636 | 2890 |
| 79 | LG:230151.1:2000SEP08 | 7066009H1 | 352 | 929 |
| 79 | LG:230151.1:2000SEP08 | 5334046H1 | 551 | 803 |
| 79 | LG:230151.1:2000SEP08 | 2991485H1 | 31 | 350 |
| 79 | LG:230151.1:2000SEP08 | 575414H1 | 22 | 274 |
| 79 | LG:230151.1:2000SEP08 | 4330089F6 | 193 | 405 |
| 79 | LG:230151.1:2000SEP08 | 4522461H1 | 2272 | 2525 |
| 80 | LG:215158.5:2000SEP08 | 4763386T8 | 1213 | 1407 |
| 80 | LG:215158.5:2000SEP08 | 7685125H1 | 857 | 1413 |
| 80 | LG:215158.5:2000SEP08 | 2049848H1 | 1197 | 1422 |
| 80 | LG:215158.5:2000SEP08 | 4521902H1 | 1212 | 1464 |
| 80 | LG:215158.5:2000SEP08 | 4786255H2 | 1251 | 1440 |
| 80 | LG:215158.5:2000SEP08 | 7685425H1 | 857 | 1429 |
| 80 | LG:215158.5:2000SEP08 | 5268360H1 | 1386 | 1567 |
| 80 | LG:215158.5:2000SEP08 | g5424826 | 1234 | 1567 |
| 80 | LG:215158.5:2000SEP08 | g4302729 | 1116 | 1566 |
| 80 | LG:215158.5:2000SEP08 | g3677733 | 1123 | 1565 |
| 80 | LG:215158.5:2000SEP08 | g3899457 | 1243 | 1562 |
| 80 | LG:215158.5:2000SEP08 | g4069202 | 1105 | 1563 |
| 80 | LG:215158.5:2000SEP08 | g3092998 | 1300 | 1563 |
| 80 | LG:215158.5:2000SEP08 | 7733923H2 | 992 | 1563 |
| 80 | LG:215158.5:2000SEP08 | g3422887 | 1118 | 1562 |
| 80 | LG:215158.5:2000SEP08 | g3178427 | 1194 | 1562 |
| 80 | LG:215158.5:2000SEP08 | g3888239 | 1147 | 1559 |
| 80 | LG:215158.5:2000SEP08 | g3418799 | 1142 | 1559 |
| 80 | LG:215158.5:2000SEP08 | g2354334 | 1103 | 1559 |
| 80 | LG:215158.5:2000SEP08 | g4763433 | 1106 | 1559 |
| 80 | LG:215158.5:2000SEP08 | 1892668H1 | 1443 | 1559 |
| 80 | LG:215158.5:2000SEP08 | 1896066H1 | 1443 | 1559 |
| 80 | LG:215158.5:2000SEP08 | 94852805 | 1188 | 1559 |
| 80 | LG:215158.5:2000SEP08 | 2288404R6 | 1151 | 1559 |
| 80 | LG:215158.5:2000SEP08 | g5811280 | 1154 | 1559 |
| 80 | LG:215158.5:2000SEP08 | 1940511H1 | 1432 | 1559 |
| 80 | LG:215158.5:2000SEP08 | g4534757 | 1095 | 1559 |
| 80 | LG:215158.5:2000SEP08 | g2751206 | 1160 | 1559 |
| 80 | LG:215158.5:2000SEP08 | 2569722R6 | 1021 | 1559 |
| 80 | LG:215158.5:2000SEP08 | g5595406 | 1393 | 1559 |
| 80 | LG:215158.5:2000SEP08 | 3367729H1 | 1166 | 1450 |
| 80 | LG:215158.5:2000SEP08 | 4116953H1 | 1126 | 1409 |
| 80 | LG:215158.5:2000SEP08 | 1421186H1 | 761 | 937 |
| 80 | LG:215158.5:2000SEP08 | 2494871H1 | 671 | 892 |
| 80 | LG:215158.5:2000SEP08 | 7756888J1 | 620 | 798 |
| 80 | LG:215158.5:2000SEP08 | 1795373H1 | 671 | 859 |
| 80 | LG:215158.5:2000SEP08 | 5412690H1 | 648 | 831 |
| 80 | LG:215158.5:2000SEP08 | 2288404H1 | 1151 | 1409 |
| 80 | LG:215158.5:2000SEP08 | 6009736H1 | 1180 | 1452 |
| 80 | LG:215158.5:2000SEP08 | 3818074H1 | 1260 | 1416 |
| 80 | LG:215158.5:2000SEP08 | 3737368H1 | 1407 | 1565 |
| 80 | LG:215158.5:2000SEP08 | 5268301H1 | 1386 | 1566 |
| 80 | LG:215158.5:2000SEP08 | 3235146H2 | 717 | 964 |
| 80 | LG:215158.5:2000SEP08 | 4423550H1 | 760 | 1011 |
| 80 | LG:215158.5:2000SEP08 | 4294737H1 | 752 | 1006 |
| 80 | LG:215158.5:2000SEP08 | 3113570H1 | 705 | 981 |
| 80 | LG:215158.5:2000SEP08 | 4298616H1 | 720 | 982 |
| 80 | LG:215158.5:2000SEP08 | 4320510H1 | 724 | 981 |
| 80 | LG:215158.5:2000SEP08 | 4312194H1 | 703 | 980 |
| 80 | LG:215158.5:2000SEP08 | 4422714H1 | 760 | 980 |
| 80 | LG:215158.5:2000SEP08 | 4709902H1 | 682 | 975 |
| 80 | LG:215158.5:2000SEP08 | 4522027H1 | 693 | 974 |
| 80 | LG:215158.5:2000SEP08 | 854561H1 | 725 | 974 |
| 80 | LG:215158.5:2000SEP08 | 5519235H1 | 830 | 966 |
| 80 | LG:215158.5:2000SEP08 | 5510860H1 | 815 | 1045 |
| 80 | LG:215158.5:2000SEP08 | 4225332H1 | 774 | 1047 |
| 80 | LG:215158.5:2000SEP08 | 5988296H1 | 762 | 1040 |
| 80 | LG:215158.5:2000SEP08 | 5653555H1 | 669 | 1043 |
| 80 | LG:215158.5:2000SEP08 | 5987596H1 | 762 | 1026 |
| 80 | LG:215158.5:2000SEP08 | 4251767H1 | 759 | 1020 |
| 80 | LG:215158.5:2000SEP08 | 4133284H2 | 767 | 1008 |
| 80 | LG:215158.5:2000SEP08 | 4435292H2 | 1223 | 1486 |
| 80 | LG:215158.5:2000SEP08 | 5025207H1 | 1213 | 1448 |
| 80 | LG:215158.5:2000SEP08 | 4978535H1 | 1223 | 1479 |
| 80 | LG:215158.5:2000SEP08 | 2601426H1 | 1252 | 1477 |
| 80 | LG:215158.5:2000SEP08 | 2639036H1 | 1232 | 1477 |
| 80 | LG:215158.5:2000SEP08 | 4977553H1 | 1250 | 1512 |
| 80 | LG:215158.5:2000SEP08 | 5853283H1 | 1255 | 1510 |
| 80 | LG:215158.5:2000SEP08 | 2181480F6 | 1048 | 1502 |
| 80 | LG:215158.5:2000SEP08 | 2181480H1 | 1215 | 1502 |
| 80 | LG:215158.5:2000SEP08 | 3210731H1 | 1241 | 1499 |
| 80 | LG:215158.5:2000SEP08 | 6129209H1 | 972 | 1493 |
| 80 | LG:215158.5:2000SEP08 | 509861H1 | 1017 | 1229 |
| 80 | LG:215158.5:2000SEP08 | 4342493H1 | 907 | 1221 |
| 80 | LG:215158.5:2000SEP08 | 7096676H1 | 725 | 1208 |
| 80 | LG:215158.5:2000SEP08 | 5965723H1 | 749 | 1199 |
| 80 | LG:215158.5:2000SEP08 | 4001315H1 | 1041 | 1194 |
| 80 | LG:215158.5:2000SEP08 | 6444667H1 | 714 | 1192 |
| 80 | LG:215158.5:2000SEP08 | g6659667 | 710 | 1120 |
| 80 | LG:215158.5:2000SEP08 | 5961576H1 | 715 | 1139 |
| 80 | LG:215158.5:2000SEP08 | 3097624H1 | 816 | 1107 |
| 80 | LG:215158.5:2000SEP08 | 5519335H1 | 830 | 1089 |
| 80 | LG:215158.5:2000SEP08 | 5564744T9 | 828 | 1336 |
| 80 | LG:215158.5:2000SEP08 | 854561R1 | 725 | 1344 |
| 80 | LG:215158.5:2000SEP08 | 1003336R1 | 888 | 1374 |
| 80 | LG:215158.5:2000SEP08 | 1003336H1 | 1100 | 1374 |
| 80 | LG:215158.5:2000SEP08 | 2569722H1 | 1021 | 1325 |
| 80 | LG:215158.5:2000SEP08 | 6890146J1 | 909 | 1316 |
| 80 | LG:215158.5:2000SEP08 | 4511922H1 | 1068 | 1310 |
| 80 | LG:215158.5:2000SEP08 | 3272493T6 | 749 | 1244 |
| 80 | LG:215158.5:2000SEP08 | 3729041H1 | 1048 | 1279 |
| 80 | LG:215158.5:2000SEP08 | 5965623H1 | 718 | 1285 |
| 80 | LG:215158.5:2000SEP08 | 4622635H1 | 1064 | 1315 |
| 80 | LG:215158.5:2000SEP08 | 4292907H1 | 1038 | 1296 |
| 80 | LG:215158.5:2000SEP08 | 4763386T9 | 850 | 1479 |
| 80 | LG:215158.5:2000SEP08 | 7685225H1 | 864 | 1489 |
| 80 | LG:215158.5:2000SEP08 | 7419422T1 | 844 | 1457 |
| 80 | LG:215158.5:2000SEP08 | 7060657H1 | 920 | 1499 |
| 80 | LG:215158.5:2000SEP08 | 4325938H1 | 1326 | 1508 |
| 80 | LG:215158.5:2000SEP08 | 4322970H1 | 1294 | 1491 |
| 80 | LG:215158.5:2000SEP08 | 4005972H1 | 1242 | 1487 |
| 80 | LG:215158.5:2000SEP08 | 5948161H1 | 1242 | 1521 |
| 80 | LG:215158.5:2000SEP08 | 94510841 | 1367 | 1555 |
| 80 | LG:215158.5:2000SEP08 | g4395334 | 1123 | 1554 |
| 80 | LG:215158.5:2000SEP08 | g5591405 | 1366 | 1553 |
| 80 | LG:215158.5:2000SEP08 | g2595148 | 1258 | 1553 |
| 80 | LG:215158.5:2000SEP08 | 5512202H1 | 1303 | 1551 |
| 80 | LG:215158.5:2000SEP08 | g2198087 | 1121 | 1536 |
| 80 | LG:215158.5:2000SEP08 | 3729041T1 | 1049 | 1522 |
| 80 | LG:215158.5:2000SEP08 | 7930783H1 | 918 | 1556 |
| 80 | LG:215158.5:2000SEP08 | 1831292H1 | 1274 | 1523 |
| 80 | LG:215158.5:2000SEP08 | 5034338T6 | 1073 | 1521 |
| 80 | LG:215158.5:2000SEP08 | 2181480T6 | 1200 | 1521 |
| 80 | LG:215158.5:2000SEP08 | 2288404T6 | 1139 | 1521 |
| 80 | LG:215158.5:2000SEP08 | 2569722T6 | 1021 | 1517 |
| 80 | LG:215158.5:2000SEP08 | 6727494H1 | 732 | 1343 |
| 80 | LG:215158.5:2000SEP08 | 1423205H1 | 1204 | 1445 |
| 80 | LG:215158.5:2000SEP08 | 6401017H1 | 1160 | 1405 |
| 80 | LG:215158.5:2000SEP08 | 5984486H1 | 1192 | 1483 |
| 80 | LG:215158.5:2000SEP08 | g4075380 | 979 | 1353 |
| 80 | LG:215158.5:2000SEP08 | 2375777H1 | 1216 | 1437 |
| 80 | LG:215158.5:2000SEP08 | 3762291H1 | 1163 | 1433 |
| 80 | LG:215158.5:2000SEP08 | g3181429 | 1153 | 1559 |
| 80 | LG:215158.5:2000SEP08 | 1896331H1 | 1443 | 1559 |
| 80 | LG:215158.5:2000SEP08 | g4004067 | 1170 | 1556 |
| 80 | LG:215158.5:2000SEP08 | 6058031H1 | 721 | 782 |
| 80 | LG:215158.5:2000SEP08 | 3097624F6 | 214 | 720 |
| 80 | LG:215158.5:2000SEP08 | 5294643H1 | 404 | 570 |
| 80 | LG:215158.5:2000SEP08 | 3097624T6 | 1 | 438 |
| 81 | LG:235840.1:2000SEP08 | 2676570F6 | 2277 | 2647 |
| 81 | LG:235840.1:2000SEP08 | 2676570T6 | 2277 | 2655 |
| 81 | LG:235840.1:2000SEP08 | g3744074 | 2254 | 2704 |
| 81 | LG:235840.1:2000SEP08 | 3992215H1 | 1800 | 2095 |
| 81 | LG:235840.1:2000SEP08 | 7193758H2 | 1810 | 2237 |
| 81 | LG:235840.1:2000SEP08 | g2836920 | 2235 | 2701 |
| 81 | LG:235840.1:2000SEP08 | 5835079H1 | 2239 | 2462 |
| 81 | LG:235840.1:2000SEP08 | g2238015 | 2338 | 2683 |
| 81 | LG:235840.1:2000SEP08 | g2737235 | 2301 | 2666 |
| 81 | LG:235840.1:2000SEP08 | g2669827 | 2286 | 2666 |
| 81 | LG:235840.1:2000SEP08 | g692242 | 2389 | 2711 |
| 81 | LG:235840.1:2000SEP08 | g2575700 | 2357 | 2637 |
| 81 | LG:235840.1:2000SEP08 | 1451436T6 | 2352 | 2667 |
| 81 | LG:235840.1:2000SEP08 | 2549073H1 | 2351 | 2602 |
| 81 | LG:235840.1:2000SEP08 | 4249979F6 | 1 | 363 |
| 81 | LG:235840.1:2000SEP08 | 942891R1 | 2442 | 2707 |
| 81 | LG:235840.1:2000SEP08 | 5187170H1 | 2459 | 2666 |
| 81 | LG:235840.1:2000SEP08 | g2727328 | 2478 | 2695 |
| 81 | LG:235840.1:2000SEP08 | g6197748 | 2554 | 2664 |
| 81 | LG:235840.1:2000SEP08 | g1391398 | 2413 | 2703 |
| 81 | LG:235840.1:2000SEP08 | 729197H1 | 2442 | 2666 |
| 81 | LG:235840.1:2000SEP08 | 942891R6 | 2442 | 2707 |
| 81 | LG:235840.1:2000SEP08 | g2705998 | 2407 | 2701 |
| 81 | LG:235840.1:2000SEP08 | g1967662 | 1129 | 1516 |
| 81 | LG:235840.1:2000SEP08 | 5286802H1 | 1076 | 1250 |
| 81 | LG:235840.1:2000SEP08 | 3595241H1 | 941 | 1230 |
| 81 | LG:235840.1:2000SEP08 | 5914590H1 | 976 | 1212 |
| 81 | LG:235840.1:2000SEP08 | 5914590F8 | 976 | 1536 |
| 81 | LG:235840.1:2000SEP08 | 7633624H1 | 586 | 1140 |
| 81 | LG:235840.1:2000SEP08 | 4725620H1 | 503 | 767 |
| 81 | LG:235840.1:2000SEP08 | g928335 | 600 | 888 |
| 81 | LG:235840.1:2000SEP08 | 4249979H1 | 1 | 247 |
| 81 | LG:235840.1:2000SEP08 | 7659126H1 | 21 | 526 |
| 81 | LG:235840.1:2000SEP08 | 4249979R6 | 89 | 580 |
| 81 | LG:235840.1:2000SEP08 | 7633624J1 | 394 | 1002 |
| 81 | LG:235840.1:2000SEP08 | 5411412H1 | 1297 | 1562 |
| 81 | LG:235840.1:2000SEP08 | 5501637F6 | 1303 | 1503 |
| 81 | LG:235840.1:2000SEP08 | 5501637H1 | 1303 | 1538 |
| 81 | LG:235840.1:2000SEP08 | g1932950 | 1379 | 1864 |
| 81 | LG:235840.1:2000SEP08 | 7613750J1 | 1195 | 1784 |
| 81 | LG:235840.1:2000SEP08 | 4243528H1 | 1218 | 1465 |
| 81 | LG:235840.1:2000SEP08 | 4664169H1 | 1222 | 1414 |
| 81 | LG:235840.1:2000SEP08 | 4664169F6 | 1222 | 1675 |
| 81 | LG:235840.1:2000SEP08 | 5511564H1 | 1222 | 1385 |
| 81 | LG:235840.1:2000SEP08 | 6306648H1 | 1230 | 1694 |
| 81 | LG:235840.1:2000SEP08 | 1451436F1 | 1500 | 1933 |
| 81 | LG:235840.1:2000SEP08 | 1451436F6 | 1500 | 2008 |
| 81 | LG:235840.1:2000SEP08 | 1451436H1 | 1500 | 1767 |
| 81 | LG:235840.1:2000SEP08 | 6160452H1 | 1530 | 1821 |
| 81 | LG:235840.1:2000SEP08 | g1389188 | 1448 | 1703 |
| 81 | LG:235840.1:2000SEP08 | 531327H1 | 1449 | 1692 |
| 81 | LG:235840.1:2000SEP08 | 6334626H1 | 1455 | 1902 |
| 81 | LG:2358A0.1:2000SEP08 | 5204143H1 | 1445 | 1709 |
| 81 | LG:235840.1:2000SEP08 | 2286077H1 | 1694 | 1932 |
| 81 | LG:235840.1:2000SEP08 | 5989717H1 | 1735 | 1985 |
| 81 | LG:235840.1:2000SEP08 | 7065831H1 | 1614 | 2104 |
| 81 | LG:235840.1:2000SEP08 | 7613750H1 | 1669 | 2267 |
| 81 | LG:235840.1:2000SEP08 | 4898449H1 | 1682 | 1867 |
| 81 | LG:235840.1:2000SEP08 | 3464906H1 | 1690 | 2022 |
| 81 | LG:235840.1:2000SEP08 | 004533H1 | 1582 | 1874 |
| 81 | LG:235840.1:2000SEP08 | 6730659H1 | 1604 | 1825 |
| 81 | LG:235840.1:2000SEP08 | 4196254H1 | 2224 | 2519 |
| 81 | LG:235840.1:2000SEP08 | 5914590R8 | 1568 | 2109 |
| 81 | LG:235840.1:2000SEP08 | 2960481H2 | 1566 | 1853 |
| 81 | LG:235840.1:2000SEP08 | 5056390H1 | 1578 | 1850 |
| 81 | LG:235840.1:2000SEP08 | 5034382T6 | 2283 | 2679 |
| 81 | LG:235840.1:2000SEP08 | 2676562H1 | 2277 | 2526 |
| 81 | LG:235840.1:2000SEP08 | 5693701H1 | 2282 | 2543 |
| 81 | LG:235840.1:2000SEP08 | 7738333H1 | 2045 | 2594 |
| 81 | LG:235840.1:2000SEP08 | 4664169T6 | 2049 | 2595 |
| 81 | LG:235840.1:2000SEP08 | 639274H1 | 2058 | 2332 |
| 81 | LG:235840.1:2000SEP08 | 544137H1 | 2149 | 2293 |
| 81 | LG:235840.1:2000SEP08 | 91270286 | 2033 | 2216 |
| 81 | LG:235840.1:2000SEP08 | 1285809H1 | 1979 | 2216 |
| 81 | LG:235840.1:2000SEP08 | 6442655H1 | 1795 | 2303 |
| 81 | LG:235840.1:2000SEP08 | 6711829H1 | 1755 | 2246 |
| 81 | LG:235840.1:2000SEP08 | 4351829H1 | 1735 | 2063 |
| 81 | LG:235840.1:2000SEP08 | 7213696H1 | 1946 | 2423 |
| 81 | LG:235840.1:2000SEP08 | g692276 | 1883 | 2221 |
| 81 | LG:235840.1:2000SEP08 | g692306 | 1883 | 1965 |
| 81 | LG:235840.1:2000SEP08 | 6377861H1 | 1919 | 2189 |
| 82 | LG:350272.1:2000SEP08 | 960820R6 | 1570 | 1941 |
| 82 | LG:350272.1:2000SEP08 | 2697244H1 | 1573 | 1858 |
| 82 | LG:350272.1:2000SEP08 | 960820H1 | 1570 | 1845 |
| 82 | LG:350272.1:2000SEP08 | 7326176H1 | 1452 | 1912 |
| 82 | LG:350272.1:2000SEP08 | 6311958H1 | 1468 | 1895 |
| 82 | LG:350272.1:2000SEP08 | 6201854H1 | 1480 | 1915 |
| 82 | LG:350272.1:2000SEP08 | 3559406H1 | 1479 | 1582 |
| 82 | LG:350272.1:2000SEP08 | 3490063H1 | 1481 | 1775 |
| 82 | LG:350272.1:20003EP08 | 5020696T1 | 1335 | 1784 |
| 82 | LG:350272.1:2000SEP08 | 2538770H1 | 1336 | 1545 |
| 82 | LG:350272.1:2000SEP08 | g2674996 | 1426 | 1831 |
| 82 | LG:350272.1:2000SEP08 | g1186534 | 1432 | 1832 |
| 82 | LG:350272.1:2000SEP08 | 603382H1 | 1336 | 1584 |
| 82 | LG:350272.1:2000SEP08 | 3740987H1 | 1339 | 1633 |
| 82 | LG:350272.1:2000SEP08 | 5020588T1 | 1354 | 1783 |
| 82 | LG:350272.1:2000SEP08 | 4241989H1 | 1420 | 1748 |
| 82 | LG:350272.1:2000SEP08 | 581813R6 | 1212 | 1561 |
| 82 | LG:350272.1:2000SEP08 | 7322258H1 | 1251 | 1868 |
| 82 | LG:350272.1:2000SEP08 | 2127622H1 | 1277 | 1536 |
| 82 | LG:350272.1:2000SEP08 | 7082642H1 | 857 | 1251 |
| 82 | LG:350272.1:20003EP08 | g1152456 | 872 | 1192 |
| 82 | LG:350272.1:2000SEP08 | g3958809 | 873 | 1219 |
| 82 | LG:350272.1:2000SEP08 | g3277672 | 873 | 1323 |
| 82 | LG:350272.1:2000SEP08 | g3249855 | 873 | 1308 |
| 82 | LG:350272.1:2000SEP08 | g1101302 | 901 | 1173 |
| 82 | LG:350272.1:2000SEP08 | g2820552 | 903 | 1337 |
| 82 | LG:350272.1:2000SEP08 | 5020696H1 | 907 | 1173 |
| 82 | LG:350272.1:2000SEP08 | 5062519H1 | 941 | 1210 |
| 82 | LG:350272.1:2000SEP08 | g3163179 | 974 | 1240 |
| 82 | LG:350272.1:2000SEP08 | 5696180H1 | 1003 | 1267 |
| 82 | LG:350272.1:2000SEP08 | 6821260J1 | 1043 | 1481 |
| 82 | LG:350272.1:2000SEP08 | 3224632H2 | 1053 | 1337 |
| 82 | LG:350272.1:2000SEP08 | 7764948H1 | 1150 | 1503 |
| 82 | LG:350272.1:2000SEP08 | 3721403H1 | 1177 | 1425 |
| 82 | LG:350272.1:2000SEP08 | 5865088H1 | 1201 | 1485 |
| 82 | LG:350272.1:2000SEP08 | 581813H1 | 1212 | 1475 |
| 82 | LG:350272.1:2000SEP08 | 4010319H1 | 2042 | 2252 |
| 82 | LG:350272.1:2000SEP08 | 1741002T6 | 2042 | 2491 |
| 82 | LG:350272.1:2000SEP08 | 2650967H1 | 2043 | 2222 |
| 82 | LG:350272.1:2000SEP08 | 401 3519H1 | 2051 | 2256 |
| 82 | LG:350272.1:2000SEP08 | 4353493H1 | 2051 | 2142 |
| 82 | LG:350272.1:2000SEP08 | 211293H1 | 2348 | 2536 |
| 82 | LG:350272.1:2000SEP08 | 4353485H1 | 2051 | 2140 |
| 82 | LG:350272.1:2000SEP08 | 211114H1 | 2052 | 2102 |
| 82 | LG:350272.1:2000SEP08 | 3621890H1 | 2051 | 2133 |
| 82 | LG:350272.1:2000SEP08 | 581813T6 | 2053 | 2491 |
| 82 | LG:350272.1:2000SEP08 | 211696H1 | 2348 | 2529 |
| 82 | LG:350272.1:2000SEP08 | g2820887 | 2054 | 2532 |
| 82 | LG:350272.1:2000SEP08 | 1684883T6 | 2062 | 2494 |
| 82 | LG:350272.1:2000SEP08 | 633648H1 | 2354 | 2542 |
| 82 | LG:350272.1:2000SEP08 | 1684883F6 | 2062 | 2529 |
| 82 | LG:350272.1:2000SEP08 | 6606673H1 | 2077 | 2530 |
| 82 | LG:350272.1:2000SEP08 | 3565543H1 | 2365 | 2487 |
| 82 | LG:350272.1:2000SEP08 | g2359505 | 2443 | 2529 |
| 82 | LG:350272.1:2000SEP08 | g4649884 | 2445 | 2523 |
| 82 | LG:350272.1:2000SEP08 | g4970896 | 2078 | 2534 |
| 82 | LG:350272.1:2000SEP08 | g4125734 | 2080 | 2518 |
| 82 | LG:350272.1:2000SEP08 | g3741618 | 2095 | 2538 |
| 82 | LG:350272.1:2000SEP08 | g6038705 | 2111 | 2529 |
| 82 | LG:350272.1:2000SEP08 | g4074869 | 2114 | 2535 |
| 82 | LG:350272.1:2000SEP08 | g5755616 | 2127 | 2535 |
| 82 | LG:350272.1:2000SEP08 | g2465965 | 2144 | 2535 |
| 82 | LG:350272.1:2000SEP08 | 92669493 | 2154 | 2531 |
| 82 | LG:350272.1:2000SEP08 | g461 8967 | 2155 | 2529 |
| 82 | LG:350272.1:2000SEP08 | g2270187 | 2176 | 2532 |
| 82 | LG:350272.1:2000SEP08 | 9855861 | 2193 | 2526 |
| 82 | LG:350272.1:2000SEP08 | g5397025 | 2183 | 2529 |
| 82 | LG:350272.1:2000SEP08 | g3307326 | 2185 | 2538 |
| 82 | LG:350272.1:2000SEP08 | 6615126H1 | 2467 | 2529 |
| 82 | LG:350272.1:2000SEP08 | g2715505 | 2186 | 2535 |
| 82 | LG:350272.1:2000SEP08 | g4267877 | 2203 | 2523 |
| 82 | LG:350272.1:2000SEP08 | g4267523 | 2203 | 2523 |
| 82 | LG:350272.1:2000SEP08 | g4267458 | 2203 | 2523 |
| 82 | LG:350272.1:2000SEP08 | 1637245H1 | 2205 | 2400 |
| 82 | LG:350272.1:2000SEP08 | 92669985 | 2196 | 2427 |
| 82 | LG:350272.1:2000SEP08 | 2561156H1 | 2221 | 2501 |
| 82 | LG:350272.1:2000SEP08 | g5364704 | 2224 | 2530 |
| 82 | LG:350272.1:2000SEP08 | 6843381H1 | 2239 | 2529 |
| 82 | LG:350272.1:2000SEP08 | g5755074 | 2262 | 2535 |
| 82 | LG:350272.1:2000SEP08 | 6326064H1 | 2271 | 2532 |
| 82 | LG:350272.1:2000SEP08 | 5306551H1 | 2276 | 2400 |
| 82 | LG:350272.1:2000SEP08 | 644238T6 | 2276 | 2495 |
| 82 | LG:350272.1:2000SEP08 | 5306583H1 | 2277 | 2429 |
| 82 | LG:350272.1:2000SEP08 | 6383176H1 | 2285 | 2491 |
| 82 | LG:350272.1:2000SEP08 | g4079566 | 2287 | 2529 |
| 82 | LG:350272.1:2000SEP08 | g2577306 | 2288 | 2529 |
| 82 | LG:350272.1:2000SEP08 | g5812197 | 2319 | 2515 |
| 82 | LG:350272.1:2000SEP08 | 1684883H1 | 2327 | 2529 |
| 82 | LG:350272.1:2000SEP08 | 3932451H1 | 2346 | 2529 |
| 82 | LG:350272.1:2000SEP08 | 6058322H1 | 1563 | 1897 |
| 82 | LG:350272.1:2000SEP08 | 4121623H1 | 1566 | 1861 |
| 82 | LG:350272.1:2000SEP08 | 1888879H1 | 1519 | 1804 |
| 82 | LG:350272.1:2000SEP08 | 4665428H1 | 1578 | 1849 |
| 82 | LG:350272.1:2000SEP08 | 708387H1 | 1595 | 1868 |
| 82 | LG:350272.1:2000SEP08 | g3213833 | 1605 | 1830 |
| 82 | LG:350272.1:2000SEP08 | 2715220H1 | 1611 | 1854 |
| 82 | LG:350272.1:2000SEP08 | g2575091 | 1627 | 1878 |
| 82 | LG:350272.1:2000SEP08 | 6486886H1 | 1625 | 2162 |
| 82 | LG:350272.1:2000SEP08 | 2206242H1 | 1627 | 1874 |
| 82 | LG:350272.1:2000SEP08 | 6821260H1 | 710 | 1191 |
| 82 | LG:350272.1:2000SEP08 | 7746167H1 | 848 | 1336 |
| 82 | LG:350272.1:2000SEP08 | 3107667H1 | 1 | 210 |
| 82 | LG:350272.1:2000SEP08 | 7674927H2 | 133 | 323 |
| 82 | LG:350272.1:2000SEP08 | 754982H 1 | 168 | 390 |
| 82 | LG:350272.1:2000SEP08 | 7346935H1 | 183 | 661 |
| 82 | LG:350272.1:2000SEP08 | g4685374 | 380 | 855 |
| 82 | LG:350272.1:2000SEP08 | g5878247 | 383 | 855 |
| 82 | LG:350272.1:2000SEP08 | 7313314H1 | 494 | 846 |
| 82 | LG:350272.1:2000SEP08 | g4649651 | 510 | 855 |
| 82 | LG:350272.1:2000SEP08 | g3191587 | 668 | 829 |
| 82 | LG:350272.1:2000SEP08 | g4889943 | 847 | 1276 |
| 82 | LG:350272.1:2000SEP08 | 3844367H1 | 1634 | 1945 |
| 82 | LG:350272.1:2000SEP08 | g2900340 | 1644 | 1819 |
| 82 | LG:350272.1:2000SEP08 | 2805489H1 | 1667 | 1917 |
| 82 | LG:350272.1:2000SEP08 | 5766360H1 | 1673 | 2184 |
| 82 | LG:350272.1:2000SEP08 | 3016843H1 | 1699 | 1895 |
| 82 | LG:350272.1:2000SEP08 | 6552652H1 | 1766 | 2282 |
| 82 | LG:350272.1:2000SEP08 | 6552052H1 | 1766 | 2225 |
| 82 | LG:350272.1:2000SEP08 | 4138187H1 | 1789 | 1895 |
| 82 | LG:350272.1:2000SEP08 | 1864387H1 | 1808 | 1895 |
| 82 | LG:350272.1:2000SEP08 | 2116744H1 | 1814 | 1895 |
| 82 | LG:350272.1:2000SEP08 | g855860 | 1834 | 2117 |
| 82 | LG:350272.1:2000SEP08 | 644363H1 | 1838 | 2105 |
| 82 | LG:350272.1:2000SEP08 | 644238R6 | 1838 | 2391 |
| 82 | LG:350272.1:2000SEP08 | 960820T6 | 2038 | 2494 |
| 82 | LG:350272.1:2000SEP08 | 1741002R6 | 2042 | 2529 |
| 82 | LG:350272.1:2000SEP08 | 644238H1 | 1838 | 1895 |
| 82 | LG:350272.1:2000SEP08 | 4003545H1 | 1838 | 2130 |
| 82 | LG:350272.1:2000SEP08 | 2511182H1 | 2025 | 2335 |
| 82 | LG:350272.1:2000SEP08 | 4010536H1 | 2032 | 2258 |
| 82 | LG:350272.1:2000SEP08 | 1741002H1 | 2042 | 2126 |
| 83 | LG:232190.1:2000SEP08 | g2167858 | 814 | 1312 |
| 83 | LG:232190.1:2000SEP08 | g1156558 | 703 | 1032 |
| 83 | LG:232190.1:2000SEP08 | 5306856H1 | 1123 | 1290 |
| 83 | LG:232190.1:2000SEP08 | g5634396 | 1222 | 1537 |
| 83 | LG:232190.1:2000SEP08 | g2167859 | 1241 | 1547 |
| 83 | LG:232190.1:2000SEP08 | g994115 | 1241 | 1454 |
| 83 | LG:232190.1:2000SEP08 | 3258260H1 | 1243 | 1531 |
| 83 | LG:232190.1:2000SEP08 | 3377873H1 | 1448 | 1636 |
| 83 | LG:232190.1:2000SEP08 | g4703402 | 1088 | 1535 |
| 83 | LG:232190.1:2000SEP08 | 5968471H1 | 846 | 1393 |
| 83 | LG:232190.1:2000SEP08 | g3918602 | 281 | 656 |
| 83 | LG:232190.1:2000SEP08 | g1138172 | 279 | 576 |
| 83 | LG:232190.1:2000SEP08 | 7069816H1 | 1 | 140 |
| 83 | LG:232190.1:2000SEP08 | 7459387H1 | 1 | 526 |
| 83 | LG:232190.1:2000SEP08 | 7594566H1 | 36 | 614 |
| 83 | LG:232190.1:2000SEP08 | 2531179F7 | 375 | 578 |
| 83 | LG:232190.1:2000SEP08 | 2531179H1 | 375 | 622 |
| 83 | LG:232190.1:2000SEP08 | 6344340H1 | 377 | 647 |
| 83 | LG:232190.1:2000SEP08 | 2729623F6 | 639 | 1097 |
| 83 | LG:232190.1:2000SEP08 | 2729623H1 | 639 | 894 |
| 83 | LG:232190.1:2000SEP08 | 7111074H1 | 500 | 934 |
| 83 | LG:232190.1:2000SEP08 | 5306756H1 | 1123 | 1282 |
| 84 | LG:1068127.1:2000SEP08 | 4914013T8 | 203 | 711 |
| 84 | LG:1068127.1:2000SEP08 | 5316376T8 | 229 | 557 |
| 84 | LG:1068127.1:2000SEP08 | 6267691F8 | 1 | 546 |
| 84 | LG:1068127.1:2000SEP08 | 6267691H1 | 1 | 492 |
| 84 | LG:1068127.1:2000SEP08 | 5491715F6 | 11 | 484 |
| 84 | LG:1068127.1:2000SEP08 | 7102152H1 | 334 | 484 |
| 84 | LG:1068127.1:2000SEP08 | 7102152F8 | 334 | 484 |
| 84 | LG:1068127.1:2000SEP08 | 7102152R8 | 334 | 492 |
| 84 | LG:1068127.1:2000SEP08 | 5316376F8 | 230 | 655 |
| 84 | LG:1068127.1:2000SEP08 | 5316376H1 | 230 | 385 |
| 85 | LG:408751.3:2000SEP08 | 5379139H1 | 434 | 679 |
| 85 | LG:408751.3:2000SEP08 | 7587582H1 | 484 | 1051 |
| 85 | LG:408751.3:2000SEP08 | 6868778H1 | 494 | 1117 |
| 85 | LG:408751.3:2000SEP08 | 7067123H1 | 525 | 1070 |
| 85 | LG:408751.3:2000SEP08 | 1270695H1 | 572 | 804 |
| 85 | LG:408751.3:2000SEP08 | 533539H1 | 427 | 622 |
| 85 | LG:408751.3:2000SEP08 | 1270695F6 | 544 | 830 |
| 85 | LG:408751.3:2000SEP08 | 2707020H1 | 588 | 881 |
| 85 | LG:408751.3:2000SEP08 | 4092963H1 | 327 | 609 |
| 85 | LG:408751.3:2000SEP08 | g677813 | 336 | 565 |
| 85 | LG:408751.3:2000SEP08 | g614162 | 336 | 605 |
| 85 | LG:408751.3:2000SEP08 | 6985794H1 | 338 | 794 |
| 85 | LG:408751.3:2000SEP08 | 7359626H1 | 379 | 948 |
| 85 | LG:408751.3:2000SEP08 | 4338771H1 | 359 | 628 |
| 85 | LG:408751.3:2000SEP08 | g708822 | 393 | 694 |
| 85 | LG:408751.3:2000SEP08 | g764692 | 395 | 736 |
| 85 | LG:408751.3:2000SEP08 | g816062 | 372 | 784 |
| 85 | LG:408751.3:2000SEP08 | 7587293H1 | 379 | 952 |
| 85 | LG:408751.3:2000SEP08 | 3864471H1 | 374 | 591 |
| 85 | LG:408751.3:2000SEP08 | 6990907H1 | 383 | 885 |
| 85 | LG:408751.3:2000SEP08 | 6866026H1 | 381 | 974 |
| 85 | LG:408751.3:2000SEP08 | 5674272H1 | 391 | 645 |
| 85 | LG:408751.3:2000SEP08 | 6120160H1 | 391 | 790 |
| 85 | LG:408751.3:2000SEP08 | 6448066H1 | 412 | 963 |
| 85 | LG:408751.3:2000SEP08 | 7580272H1 | 393 | 907 |
| 85 | LG:408751.3:2000SEP08 | g691925 | 443 | 755 |
| 85 | LG:408751.3:2000SEP08 | 533539R6 | 424 | 939 |
| 85 | LG:408751.3:2000SEP08 | 4705993H1 | 1999 | 2149 |
| 85 | LG:408751.3:2000SEP08 | 7158434H1 | 2001 | 2270 |
| 85 | LG:408751.3:2000SEP08 | 6983326H1 | 2077 | 2666 |
| 85 | LG:408751.3:2000SEP08 | 6770575H1 | 2300 | 2823 |
| 85 | LG:408751.3:2000SEP08 | 6765966H1 | 2133 | 2654 |
| 85 | LG:408751.3:2000SEP08 | 7580816H1 | 2166 | 2665 |
| 85 | LG:408751.3:2000SEP08 | 1270536H1 | 2175 | 2415 |
| 85 | LG:408751.3:2000SEP08 | 7281449H1 | 2232 | 2815 |
| 85 | LG:408751.3:2000SEP08 | 7582690H1 | 2242 | 2758 |
| 85 | LG:408751.3:2000SEP08 | 7275431J1 | 2370 | 2836 |
| 85 | LG:408751.3:2000SEP08 | 5920291H1 | 208 | 267 |
| 85 | LG:408751.3:2000SEP08 | 1456735F6 | 189 | 605 |
| 85 | LG:408751.3:2000SEP08 | 6721132H1 | 193 | 579 |
| 85 | LG:408751.3:2000SEP08 | 4203426H1 | 212 | 337 |
| 85 | LG:408751.3:2000SEP08 | 1992224H1 | 206 | 475 |
| 85 | LG:408751.3:2000SEP08 | 7259028H1 | 204 | 579 |
| 85 | LG:408751.3:2000SEP08 | g766593 | 289 | 587 |
| 85 | LG:408751.3:2000SEP08 | 7058996H1 | 309 | 890 |
| 85 | LG:408751.3:2000SEP08 | g570855 | 2997 | 3307 |
| 85 | LG:408751.3:2000SEP08 | 5371992H1 | 3015 | 3187 |
| 85 | LG:408751.3:2000SEP08 | g389944 | 3100 | 3330 |
| 85 | LG:408751.3:2000SEP08 | 2416693F6 | 3191 | 3330 |
| 85 | LG:408751.3:2000SEP08 | 2416693H1 | 3191 | 3330 |
| 85 | LG:408751.3:2000SEP08 | g318775 | 3202 | 3330 |
| 85 | LG:408751.3:2000SEP08 | 1594111H1 | 3236 | 3330 |
| 85 | LG:408751.3:2000SEP08 | g389942 | 3242 | 3330 |
| 85 | LG:408751.3:2000SEP08 | 5406949T6 | 1495 | 1919 |
| 85 | LG:408751.3:2000SEP08 | 5945223H1 | 1574 | 1656 |
| 85 | LG:408751.3:2000SEP08 | 6773005H1 | 1585 | 2157 |
| 85 | LG:408751.3:2000SEP08 | 6869723H1 | 1599 | 1944 |
| 85 | LG:408751.3:2000SEP08 | g2985356 | 1730 | 1957 |
| 85 | LG:408751.3:2000SEP08 | 6768978H1 | 1703 | 2265 |
| 85 | LG:408751.3:2000SEP08 | 2154505H1 | 1905 | 2188 |
| 85 | LG:408751.3:2000SEP08 | 7585176H1 | 1906 | 2405 |
| 85 | LG:408751.3:2000SEP08 | 2154505F7 | 1906 | 2204 |
| 85 | LG:408751.3:2000SEP08 | 6871487H1 | 1928 | 2449 |
| 85 | LG:408751.3:2000SEP08 | g2003420 | 1937 | 2178 |
| 85 | LG:408751.3:2000SEP08 | g5553670 | 1949 | 2370 |
| 85 | LG:408751.3:2000SEP08 | 1456735H1 | 208 | 332 |
| 85 | LG:408751.3:2000SEP08 | g677040 | 204 | 322 |
| 85 | LG:408751.3:2000SEP08 | g1950097 | 237 | 294 |
| 85 | LG:408751.3:2000SEP08 | 7290347H1 | 188 | 672 |
| 85 | LG:408751.3:2000SEP08 | 7290834H1 | 187 | 505 |
| 85 | LG:408751.3:2000SEP08 | g4152281 | 207 | 277 |
| 85 | LG:408751.3:2000SEP08 | g1627181 | 208 | 330 |
| 85 | LG:408751.3:2000SEP08 | 1853628H1 | 181 | 421 |
| 85 | LG:408751.3:2000SEP08 | 3944530H1 | 184 | 461 |
| 85 | LG:408751.3:2000SEP08 | 1265660H1 | 181 | 469 |
| 85 | LG:408751.3:2000SEP08 | 7411039H1 | 2627 | 3182 |
| 85 | LG:408751.3:2000SEP08 | 1270292F6 | 2718 | 2996 |
| 85 | LG:408751.3:2000SEP08 | 1270292H1 | 2718 | 2955 |
| 85 | LG:408751.3:2000SEP08 | 6984280R8 | 2735 | 3167 |
| 85 | LG:408751.3:2000SEP08 | 6984280H1 | 2735 | 3103 |
| 85 | LG:408751.3:2000SEP08 | 6984280F8 | 2736 | 3174 |
| 85 | LG:408751.3:2000SEP08 | 6985157R8 | 2882 | 3147 |
| 85 | LG:408751.3:2000SEP08 | 6985157H1 | 2882 | 3147 |
| 85 | LG:408751.3:2000SEP08 | g1991410 | 2636 | 2766 |
| 85 | LG:408751.3:2000SEP08 | g1044150 | 2643 | 2837 |
| 85 | LG:408751.3:2000SEP08 | 6985157F8 | 2882 | 3146 |
| 85 | LG:408751.3:2000SEP08 | g616005 | 2959 | 3306 |
| 85 | LG:408751.3:2000SEP08 | 4203459F6 | 2962 | 3331 |
| 85 | LG:408751.3:2000SEP08 | 6508635H1 | 2987 | 3173 |
| 85 | LG:408751.3:2000SEP08 | 7360836H1 | 2990 | 3331 |
| 85 | LG:408751.3:2000SEP08 | 6447490H1 | 2382 | 2954 |
| 85 | LG:408751.3:2000SEP08 | 7581728H1 | 2401 | 2932 |
| 85 | LG:408751.3:2000SEP08 | 5922289H1 | 2401 | 2668 |
| 85 | LG:408751.3:2000SEP08 | 7188176H1 | 2446 | 3010 |
| 85 | LG:408751.3:2000SEP08 | 574501H1 | 2482 | 2729 |
| 85 | LG:408751.3:2000SEP08 | 3460095H1 | 2587 | 2837 |
| 85 | LG:408751.3:2000SEP08 | 7583983H1 | 2588 | 3127 |
| 85 | LG:408751.3:2000SEP08 | 1270279H1 | 2603 | 2823 |
| 85 | LG:408751.3:2000SEP08 | g615297 | 1339 | 1671 |
| 85 | LG:408751.3:2000SEP08 | g517687 | 1339 | 1669 |
| 85 | LG:408751.3:2000SEP08 | g615578 | 1385 | 1671 |
| 85 | LG:408751.3:2000SEP08 | g614283 | 1381 | 1664 |
| 85 | LG:408751.3:2000SEP08 | 7361584H1 | 1393 | 1931 |
| 85 | LG:408751.3:2000SEP08 | 1456735T6 | 1430 | 1632 |
| 85 | LG:408751.3:2000SEP08 | g4328099 | 1512 | 1729 |
| 85 | LG:408751.3:2000SEP08 | g614262 | 1457 | 1664 |
| 85 | LG:408751.3:2000SEP08 | g4152278 | 1463 | 1663 |
| 85 | LG:408751.3:2000SEP08 | g562532 | 1469 | 1664 |
| 85 | LG:408751.3:2000SEP08 | g671207 | 1470 | 1664 |
| 85 | LG:408751.3:2000SEP08 | 5407853T6 | 1462 | 1920 |
| 85 | LG:408751.3:2000SEP08 | g4148449 | 1491 | 1946 |
| 85 | LG:408751.3:2000SEP08 | 7275431H2 | 1463 | 2036 |
| 85 | LG:408751.3:2000SEP08 | 6572506H1 | 1480 | 2041 |
| 85 | LG:408751.3:2000SEP08 | 7410777H1 | 1480 | 2021 |
| 85 | LG:408751.3:2000SEP08 | g5511164 | 1289 | 1671 |
| 85 | LG:408751.3:2000SEP08 | g3649444 | 1290 | 1673 |
| 85 | LG:408751.3:2000SEP08 | g314750 | 1302 | 1671 |
| 85 | LG:408751.3:2000SEP08 | g775420 | 1276 | 1685 |
| 85 | LG:408751.3:2000SEP08 | g4617815 | 1287 | 1678 |
| 85 | LG:408751.3:2000SEP08 | g314920 | 1339 | 1671 |
| 85 | LG:408751.3:2000SEP08 | 1270695T6 | 1192 | 1632 |
| 85 | LG:408751.3:2000SEP08 | 4717574T6 | 1201 | 1650 |
| 85 | LG:408751.3:2000SEP08 | 1476570F6 | 1203 | 1671 |
| 85 | LG:408751.3:2000SEP08 | 1476571F6 | 1203 | 1547 |
| 85 | LG:408751.3:2000SEP08 | 1476570H1 | 1203 | 1409 |
| 85 | LG:408751.3:2000SEP08 | g614326 | 1215 | 1678 |
| 85 | LG:408751.3:2000SEP08 | 1476571T6 | 1221 | 1634 |
| 85 | LG:408751.3:2000SEP08 | g4152280 | 1234 | 1403 |
| 85 | LG:408751.3:2000SEP08 | g4598685 | 1244 | 1672 |
| 85 | LG:408751.3:2000SEP08 | 2153570H1 | 1256 | 1530 |
| 85 | LG:408751.3:2000SEP08 | g314775 | 1258 | 1671 |
| 85 | LG:408751.3:2000SEP08 | 4492503H1 | 1264 | 1672 |
| 85 | LG:408751.3:2000SEP08 | g61 5988 | 1270 | 1671 |
| 85 | LG:408751.3:2000SEP08 | g4223790 | 827 | 1266 |
| 85 | LG:408751.3:2000SEP08 | 5407853F6 | 814 | 1181 |
| 85 | LG:408751.3:2000SEP08 | 6717166H1 | 833 | 1295 |
| 85 | LG:408751.3:2000SEP08 | 7160561H1 | 835 | 1188 |
| 85 | LG:408751.3:2000SEP08 | g3331126 | 848 | 1268 |
| 85 | LG:408751.3:2000SEP08 | 5310872H1 | 850 | 1076 |
| 85 | LG:408751.3:2000SEP08 | 73881 16H1 | 852 | 1314 |
| 85 | LG:408751.3:2000SEP08 | g6992853 | 862 | 1266 |
| 85 | LG:408751.3:2000SEP08 | 5267191H1 | 869 | 1129 |
| 85 | LG:408751.3:20003EP08 | 4940779H1 | 890 | 1162 |
| 85 | LG:408751.3:2000SEP08 | 1270258H1 | 892 | 1130 |
| 85 | LG:408751.3:2000SEP08 | g794503 | 898 | 1291 |
| 85 | LG:408751.3:2000SEP08 | g816007 | 896 | 1257 |
| 85 | LG:408751.3:2000SEP08 | g901436 | 904 | 1267 |
| 85 | LG:408751.3:2000SEP08 | 5205391H1 | 597 | 836 |
| 85 | LG:408751.3:2000SEP08 | 5498383F6 | 573 | 1056 |
| 85 | LG:408751.3:2000SEP08 | 5498383H1 | 573 | 811 |
| 85 | LG:408751.3:2000SEP08 | 5311056H1 | 591 | 753 |
| 85 | LG:408751.3:2000SEP08 | 7372949H1 | 644 | 1194 |
| 85 | LG:408751.3:2000SEP08 | 5907142H1 | 671 | 950 |
| 85 | LG:408751.3:2000SEP08 | 5924427H1 | 693 | 983 |
| 85 | LG:408751.3:2000SEP08 | 6869327H1 | 736 | 1241 |
| 85 | LG:408751.3:2000SEP08 | 5406949H1 | 814 | 1046 |
| 85 | LG:408751.3:2000SEP08 | 6984258H1 | 733 | 1127 |
| 85 | LG:408751.3:2000SEP08 | 5406949F6 | 814 | 981 |
| 85 | LG408751.3:2000SEP08 | 009349H1 | 773 | 1119 |
| 85 | LG:408751.3:2000SEP08 | 6888770H1 | 784 | 1299 |
| 85 | LG:408751.3:2000SEP08 | 4943311T6 | 797 | 1243 |
| 85 | LG:408751.3:2000SEP08 | 7279978H1 | 787 | 1322 |
| 85 | LG:408751.3:2000SEP08 | 7292792H1 | 805 | 1378 |
| 85 | LG:408751.3:2000SEP08 | g1192539 | 814 | 1266 |
| 85 | LG:408751.3:2000SEP08 | 6037020H1 | 802 | 1344 |
| 85 | LG:408751.3:2000SEP08 | 5407853H1 | 814 | 940 |
| 85 | LG:408751.3:2000SEP08 | 4705993T9 | 1119 | 1569 |
| 85 | LG:408751.3:2000SEP08 | 004952H1 | 1179 | 1438 |
| 85 | LG:408751.3:2000SEP08 | 1476570T6 | 1187 | 1631 |
| 85 | LG:408751.3:2000SEP08 | 748579H1 | 1095 | 1335 |
| 85 | LG:408751.3:2000SEP08 | 748579R1 | 1091 | 1668 |
| 85 | LG:408751.3:2000SEP08 | 859218T6 | 1028 | 1600 |
| 85 | LG:408751.3:2000SEP08 | 6855475H1 | 1028 | 1225 |
| 85 | LG:408751.3:2000SEP08 | 6871051H1 | 1080 | 1596 |
| 85 | LG:408751.3:2000SEP08 | 1270292T6 | 1031 | 1592 |
| 85 | LG:408751.3:2000SEP08 | g822109 | 1041 | 1250 |
| 85 | LG:408751.3:2000SEP08 | 2416693T6 | 1106 | 1626 |
| 85 | LG:408751.3:2000SEP08 | g6086997 | 914 | 1274 |
| 85 | LG:408751.3:2000SEP08 | 533539T6 | 921 | 1238 |
| 85 | LG:408751.3:2000SEP08 | 5371992T9 | 928 | 1565 |
| 85 | LG:408751.3:2000SEP08 | g314842 | 931 | 1237 |
| 85 | LG:408751.3:2000SEP08 | g683067 | 953 | 1236 |
| 85 | LG:408751.3:2000SEP08 | 7290682H1 | 961 | 1496 |
| 85 | LG:408751.3:2000SEP08 | 859218R1 | 990 | 1510 |
| 85 | LG:408751.3:2000SEP08 | 859218H1 | 990 | 1204 |
| 85 | LG:408751.3:2000SEP08 | 859218R6 | 990 | 1432 |
| 85 | LG:408751.3:2000SEP08 | g567610 | 995 | 1237 |
| 85 | LG:408751.3:2000SEP08 | 4943311H1 | 175 | 458 |
| 85 | LG:408751.3:2000SEP08 | 4943311F6 | 175 | 595 |
| 85 | LG:408751.3:2000SEP08 | 6818987H1 | 197 | 267 |
| 85 | LG:408751.3:2000SEP08 | 1265660F1 | 181 | 790 |
| 85 | LG:408751.3:2000SEP08 | g1978747 | 1 | 307 |
| 85 | LG:408751.3:2000SEP08 | g5553287 | 1 | 315 |
| 85 | LG:408751.3:2000SEP08 | 6818987J1 | 33 | 250 |
| 85 | LG:408751.3:2000SEP08 | 6989857H1 | 1 | 436 |
| 85 | LG:408751.3:2000SEP08 | 6955370H1 | 22 | 540 |
| 85 | LG:408751.3:2000SEP08 | g6701393 | 25 | 503 |
| 85 | LG:408751.3:2000SEP08 | g4390046 | 24 | 500 |
| 85 | LG:408751.3:2000SEP08 | g4534562 | 24 | 504 |
| 85 | LG:408751.3:2000SEP08 | g1192915 | 25 | 170 |
| 85 | LG:408751.3:2000SEP08 | g2003054 | 31 | 344 |
| 85 | LG:408751.3:2000SEP08 | 6770575J1 | 35 | 555 |
| 85 | LG:408751.3:2000SEP08 | 6572506J1 | 33 | 460 |
| 85 | LG:408751.3:2000SEP08 | 6768978J1 | 33 | 631 |
| 85 | LG:408751.3:2000SEP08 | 6765966J1 | 33 | 606 |
| 85 | LG:408751.3:2000SEP08 | 6773005J1 | 33 | 637 |
| 85 | LG:408751.3:2000SEP08 | 6818431J1 | 33 | 570 |
| 85 | LG:408751.3:2000SEP08 | g2003419 | 45 | 421 |
| 85 | LG:408751.3:2000SEP08 | g1551472 | 61 | 213 |
| 85 | LG:408751.3:2000SEP08 | 6147606H1 | 71 | 625 |
| 85 | LG:408751.3:2000SEP08 | g615579 | 115 | 462 |
| 85 | LG:408751.3:2000SEP08 | g389770 | 122 | 510 |
| 85 | LG:408751.3:2000SEP08 | 5311056F8 | 141 | 753 |
| 85 | LG:408751.3:2000SEP08 | 688877W 1 | 153 | 753 |
| 85 | LG:408751.3:2000SEP08 | g615989 | 174 | 503 |
| 86 | LG:1078933.1:2000SEP08 | 2730285H1 | 654 | 890 |
| 86 | LG:1078933.1:2000SEP08 | g2728816 | 753 | 922 |
| 86 | LG:1078933.1:2000SEP08 | 4002923H1 | 773 | 1072 |
| 86 | LG:1078933.1:2000SEP08 | g2537503 | 800 | 1089 |
| 86 | LG:1078933.1:2000SEP08 | g6299364 | 1051 | 1124 |
| 86 | LG:1078933.1:2000SEP08 | 4992604T6 | 442 | 970 |
| 86 | LG:1078933.1:2000SEP08 | 2795327H1 | 459 | 701 |
| 86 | LG:1078933.1:2000SEP08 | 6000894T8 | 511 | 998 |
| 86 | LG:1078933.1:2000SEP08 | 4992604F6 | 1 561 | |
| 86 | LG:1078933.1:2000SEP08 | 4992604H1 | 1 | 207 |
| 86 | LG:1078933.1:2000SEP08 | 7264011H1 | 45 | 555 |
| 86 | LG:1078933.1:2000SEP08 | 4251691H1 | 398 | 656 |
| 86 | LG:1078933.1:2000SEP08 | 5305837H1 | 410 | 651 |
| 86 | LG:1078933.1:2000SEP08 | 2651913F6 | 438 | 990 |
| 86 | LG:1078933.1:2000SEP08 | 2651913H1 | 438 | 683 |
| 86 | LG:1078933.1:2000SEP08 | 1720617T6 | 592 | 1054 |
| 86 | LG:1078933.1:2000SEP08 | 4030861T6 | 612 | 1166 |
| 87 | LG:958731.1:2000SEP08 | 6274461H2 | 1 | 390 |
| 87 | LG:958731.1:2000SEP08 | 6274461F8 | 1 | 586 |
| 87 | LG:958731.1:2000SEP08 | 6274461T8 | 394 | 1095 |
| 88 | LG:024125.5:2000SEP08 | 7937162H1 | 1 | 577 |
| 88 | LG:024125.5:2000SEP08 | 3359165H1 | 14 | 254 |
| 88 | LG:024125.5:2000SEP08 | 927867H1 | 28 | 176 |
| 88 | LG:024125.5:2000SEP08 | 4729644H1 | 32 | 297 |
| 88 | LG:024125.5:2000SEP08 | g5430947 | 34 | 309 |
| 88 | LG:024125.5:2000SEP08 | g6300785 | 626 | 783 |
| 88 | LG:024125.5:2000SEP08 | g3918107 | 627 | 783 |
| 88 | LG:024125.5:2000SEP08 | 1848687T6 | 640 | 818 |
| 88 | LG:024125.5:2000SEP08 | g4078394 | 641 | 783 |
| 88 | LG:024125.5:2000SEP08 | g6301603 | 646 | 783 |
| 88 | LG:024125.5:2000SEP08 | 94109417 | 671 | 783 |
| 88 | LG:024125.5:2000SEP08 | 3799006H1 | 673 | 805 |
| 88 | LG:024125.5:2000SEP08 | g5531063 | 676 | 783 |
| 88 | LG:024125.5:2000SEP08 | g2115664 | 677 | 783 |
| 88 | LG:024125.5:2000SEP08 | g4004611 | 682 | 783 |
| 88 | LG:024125.5:2000SEP08 | g3417887 | 690 | 783 |
| 88 | LG:024125.5:2000SEP08 | g3422421 | 690 | 783 |
| 88 | LG:024125.5:2000SEP08 | g1141030 | 716 | 783 |
| 88 | LG:024125.5:2000SEP08 | g3078258 | 744 | 820 |
| 88 | LG:024125.5:2000SEP08 | g873980 | 752 | 809 |
| 88 | LG:024125.5:2000SEP08 | g5431328 | 622 | 805 |
| 88 | LG:024125.5:2000SEP08 | 1528650H1 | 625 | 829 |
| 88 | LG:024125.5:2000SEP08 | 3246738H1 | 568 | 823 |
| 88 | LG:024125.5:2000SEP08 | 4544253H1 | 571 | 808 |
| 88 | LG:024125.5:2000SEP08 | 4329943H1 | 557 | 759 |
| 88 | LG:024125.5:2000SEP08 | 4270595H1 | 567 | 820 |
| 88 | LG:024125.5:2000SEP08 | 2995696H1 | 549 | 800 |
| 88 | LG:024125.5:2000SEP08 | 4330031H1 | 557 | 745 |
| 88 | LG:024125.5:2000SEP08 | g6713522 | 513 | 783 |
| 88 | LG:024125.5:2000SEP08 | 6141768H1 | 523 | 794 |
| 88 | LG:024125.5:2000SEP08 | 2994610H1 | 549 | 783 |
| 88 | LG:024125.5:2000SEP08 | 4625455H1 | 501 | 739 |
| 88 | LG:024125.5:2000SEP08 | g6300249 | 39 | 447 |
| 88 | LG:024125.5:2000SEP08 | 1391910H1 | 39 | 303 |
| 88 | LG:024125.5:2000SEP08 | 1439283F6 | 39 | 660 |
| 88 | LG:024125.5:2000SEP08 | 927867R1 | 35 | 490 |
| 88 | LG:024125.5:2000SEP08 | 3366477H1 | 35 | 308 |
| 88 | LG:024125.5:2000SEP08 | 986481H1 | 47 | 334 |
| 88 | LG:024125.5:2000SEP08 | 3537126H1 | 45 | 215 |
| 88 | LG:024125.5:2000SEP08 | 986481R1 | 47 | 350 |
| 88 | LG:024125.5:2000SEP08 | 2729856H1 | 48 | 294 |
| 88 | LG:024125.5:2000SEP08 | 630784H1 | 48 | 135 |
| 88 | LG:024125.5:2000SEP08 | 1235980H1 | 47 | 273 |
| 88 | LG:024125.5:2000SEP08 | 4666859H1 | 48 | 292 |
| 88 | LG:024125.5:2000SEP08 | 1414985H1 | 45 | 280 |
| 88 | LG:024125.5:2000SEP08 | 1414984H1 | 45 | 284 |
| 88 | LG:024125.5:2000SEP08 | 2279387H1 | 48 | 332 |
| 88 | LG:024125.5:2000SEP08 | 1235980F1 | 47 | 597 |
| 88 | LG:024125.5:2000SEP08 | 1334045H1 | 42 | 186 |
| 88 | LG:024125.5:2000SEP08 | 3387780H1 | 44 | 342 |
| 88 | LG:024125.5:2000SEP08 | g677179 | 42 | 278 |
| 88 | LG:024125.5:2000SEP08 | g874037 | 43 | 380 |
| 88 | LG:024125.5:2000SEP08 | 4887675H1 | 45 | 173 |
| 88 | LG:024125.5:2000SEP08 | 3401859H1 | 39 | 267 |
| 88 | LG:024125.5:2000SEP08 | 4747105H1 | 42 | 322 |
| 88 | LG:024125.5:2000SEP08 | 2701713H1 | 40 | 310 |
| 88 | LG:024125.5:2000SEP08 | 2519203H1 | 40 | 275 |
| 88 | LG:024125.5:2000SEP08 | 4017658H1 | 41 | 293 |
| 88 | LG:024125.5:2000SEP08 | 4843176H1 | 42 | 316 |
| 88 | LG:024125.5:2000SEP08 | g6300000 | 39 | 470 |
| 88 | LG:024125.5:2000SEP08 | 896173H1 | 245 | 483 |
| 88 | LG:024125.5:2000SEP08 | 4333283H1 | 264 | 537 |
| 88 | LG:024125.5:2000SEP08 | 7416919T1 | 319 | 827 |
| 88 | LG:024125.5:2000SEP08 | 5299466H1 | 449 | 716 |
| 88 | LG:024125.5:2000SEP08 | 896173R1 | 245 | 815 |
| 88 | LG:024125.5:2000SEP08 | 2134896H1 | 201 | 460 |
| 88 | LG:024125.5:2000SEP08 | 7437029H1 | 144 | 631 |
| 88 | LG:024125.5:2000SEP08 | 1399836H1 | 160 | 400 |
| 88 | LG:024125.5:2000SEP08 | g2011988 | 109 | 268 |
| 88 | LG:024125.5:2000SEP08 | 6497615H1 | 130 | 683 |
| 88 | LG:024125.5:2000SEP08 | g2111895 | 48 | 449 |
| 88 | LG:024125.5:2000SEP08 | 2453432H1 | 82 | 347 |
| 88 | LG:024125.5:2000SEP08 | 3699972H1 | 48 | 336 |
| 88 | LG:024125.5:2000SEP08 | g1810337 | 51 | 427 |
| 88 | LG:024125.5:2000SEP08 | g1544548 | 49 | 170 |
| 88 | LG:024125.5:2000SEP08 | g1163380 | 48 | 406 |
| 89 | LG:373637.3:2000SEP08 | g3040122 | 647 | 903 |
| 89 | LG:373637.3:2000SEP08 | g2942533 | 587 | 903 |
| 89 | LG:373637.3:2000SEP08 | g6041238 | 605 | 900 |
| 89 | LG:373637.3:2000SEP08 | g3051904 | 649 | 900 |
| 89 | LG:373637.3:2000SEP08 | g3804542 | 400 | 782 |
| 89 | LG:373637.3:2000SEP08 | 6245663F8 | 1 | 684 |
| 89 | LG:373637.3:2000SEP08 | 6245663H1 | 1 | 508 |
| 89 | LG:373637.3:2000SEP08 | g3180013 | 567 | 1011 |
| 89 | LG:373637.3:2000SEP08 | g3594985 | 588 | 1008 |
| 89 | LG:373637.3:2000SEP08 | g5675509 | 577 | 1004 |
| 89 | LG:373637.3:2000SEP08 | 6245663T8 | 237 | 904 |
| 89 | LG:373637.3:2000SEP08 | g2953832 | 641 | 903 |
| 89 | LG:373637.3:2000SEP08 | g6036927 | 493 | 903 |
| 89 | LG:373637.3:2000SEP08 | g5394478 | 447 | 903 |
| 90 | LG:1053229.1:2000SEP08 | 1445465F6 | 56 | 334 |
| 90 | LG:1053229.1:2000SEP08 | 6103338F7 | 66 | 595 |
| 90 | LG:1053229.1:2000SEP08 | 1445465H1 | 53 | 322 |
| 90 | LG:1053229.1:2000SEP08 | 1259655 | 1 | 217 |
| 90 | LG:1053229.1:2000SEP08 | g1439745 | 13 | 308 |
| 90 | LG:1053229.1:2000SEP08 | 6103338H1 | 66 | 376 |
| 90 | LG:1053229.1:2000SEP08 | 1415866T6 | 393 | 587 |
| 90 | LG:1053229.1:2000SEP08 | g1442104 | 1 | 292 |
| 91 | LG:248364.1:2000SEP08 | 7363704H1 | 621 | 1110 |
| 91 | LG:248364.1:2000SEP08 | 7041822R8 | 693 | 1335 |
| 91 | LG:248364.1:2000SEP08 | 7468175H1 | 456 | 898 |
| 91 | LG:248364.1:2000SEP08 | 6758436H1 | 1100 | 1572 |
| 91 | LG:248364.1:2000SEP08 | 6353449F7 | 1048 | 1595 |
| 91 | LG:248364.1:2000SEP08 | 6608287H1 | 1244 | 1796 |
| 91 | LG:248364.1:2000SEP08 | 7754413H1 | 1124 | 1659 |
| 91 | LG:248364.1:2000SEP08 | 814363R1 | 1500 | 1930 |
| 91 | LG:248364.1:2000SEP08 | 814363R6 | 1500 | 1854 |
| 91 | LG:248364.1:2000SEP08 | 5047370H1 | 1447 | 1737 |
| 91 | LG:248364.1:2000SEP08 | 7608161J1 | 1374 | 1930 |
| 91 | LG:248364.1:2000SEP08 | 3071079H1 | 1364 | 1661 |
| 91 | LG:248364.1:2000SEP08 | g3931760 | 1512 | 1907 |
| 91 | LG:248364.1:2000SEP08 | 814363H1 | 1500 | 1729 |
| 91 | LG:248364.1:2000SEP08 | 7092325H1 | 146 | 384 |
| 91 | LG:248364.1:2000SEP08 | 6567819H1 | 146 | 688 |
| 91 | LG:248364.1:2000SEP08 | 6567819F6 | 146 | 606 |
| 91 | LG:248364.1:2000SEP08 | 6776971J1 | 1 | 596 |
| 91 | LG:248364.1:2000SEP08 | 93873143 | 220 | 416 |
| 91 | LG:248364.1:2000SEP08 | g1067627 | 187 | 516 |
| 91 | LG:248364.1:2000SEP08 | g6575611 | 182 | 600 |
| 91 | LG:248364.1:2000SEP08 | g3734235 | 180 | 494 |
| 91 | LG:248364.1:2000SEP08 | 7608161H1 | 805 | 1211 |
| 91 | LG:248364.1:2000SEP08 | 7384566H1 | 843 | 1160 |
| 91 | LG:248364.1:2000SEP08 | 7097046H1 | 1009 | 1496 |
| 92 | LG:477130.1:2000SEP08 | 5910620H1 | 1 | 302 |
| 92 | LG:477130.1:2000SEP08 | 5910620F8 | 1 | 460 |
| 92 | LG:477130.1:2000SEP08 | 5910668H1 | 2 | 301 |
| 92 | LG:477130.1:2000SEP08 | 5910620T9 | 280 | 764 |
| 92 | LG:477130.1:2000SEP08 | 5910620T6 | 480 | 889 |
| 93 | LG:113786.17:2000SEP08 | 2441606F6 | 1 | 496 |
| 93 | LG:113786.17:2000SEP08 | 2441606H1 | 1 | 218 |
| 93 | LG:113786.17:2000SEP08 | 2472557H1 | 33 | 262 |
| 93 | LG:113786.17:2000SEP08 | 2472557F6 | 33 | 395 |
| 94 | LG:347635.1:2000SEP08 | 5639879H1 | 58 | 292 |
| 94 | LG:347635.1:2000SEP08 | 5639879F6 | 58 | 586 |
| 94 | LG:347635.1:2000SEP08 | 7186104H1 | 1 | 542 |
| 94 | LG:347635.1:2000SEP08 | g2359789 | 585 | 1021 |
| 94 | LG:347635.1:2000SEP08 | 7714779H1 | 677 | 1165 |
| 94 | LG:347635.1:2000SEP08 | g274093 | 691 | 991 |
| 94 | LG:347635.1:2000SEP08 | 5699927F6 | 1064 | 1517 |
| 94 | LG:347635.1:2000SEP08 | g2743365 | 457 | 916 |
| 94 | LG:347635.1:2000SEP08 | 7152112H1 | 582 | 1003 |
| 94 | LG:347635.1:2000SEP08 | 6777521J1 | 235 | 837 |
| 94 | LG:347635.1:2000SEP08 | 7714779J1 | 23 | 594 |
| 94 | LG:347635.1:2000SEP08 | 7757195J1 | 1 | 542 |
| 94 | LG:347635.1:2000SEP08 | 7757195H1 | 89 | 667 |
| 94 | LG:347635.1:2000SEP08 | 5699927H1 | 1065 | 1313 |
| 95 | LG:242966.4:2000SEP08 | 568455H1 | 1669 | 1916 |
| 95 | LG:242966.4:2000SEP08 | 2512929T6 | 1690 | 1934 |
| 95 | LG:242966.4:2000SEP08 | 2196270H1 | 1716 | 1926 |
| 95 | LG:242966.4:2000SEP08 | 1660835H1 | 1719 | 1939 |
| 95 | LG:242966.4:2000SEP08 | 1538251H1 | 1742 | 1964 |
| 95 | LG:242966.4:2000SEP08 | 600839H1 | 1755 | 2010 |
| 95 | LG:242966.4:2000SEP08 | 039755H1 | 1920 | 2026 |
| 95 | LG:242966.4:2000SEP08 | g3329925 | 1786 | 1973 |
| 95 | LG:242966.4:2000SEP08 | g2524293 | 1878 | 1941 |
| 95 | LG:242966.4:2000SEP08 | g6133327 | 1668 | 1982 |
| 95 | LG:242966.4:2000SEP08 | 1417037H1 | 1658 | 1903 |
| 95 | LG:242966.4:2000SEP08 | 1417037F6 | 1658 | 2026 |
| 95 | LG:242966.4:2000SEP08 | 2597301T6 | 1602 | 1937 |
| 95 | LG:242966.4:2000SEP08 | 983791H1 | 1606 | 1903 |
| 95 | LG:242966.4:2000SEP08 | g6701826 | 1609 | 1973 |
| 95 | LG:242966.4:2000SEP08 | 1383448T6 | 1610 | 1933 |
| 95 | LG:242966.4:2000SEP08 | 3282241H1 | 1628 | 1865 |
| 95 | LG:242966.4:2000SEP08 | 5098821H1 | 1634 | 1895 |
| 95 | LG:242966.4:2000SEP08 | g4648103 | 1652 | 1973 |
| 95 | LG:242966.4:2000SEP08 | g5836941 | 1652 | 1973 |
| 95 | LG:242966.4:2000SEP08 | g5741352 | 1652 | 1977 |
| 95 | LG:242966.4:2000SEP08 | 5817026H1 | 1524 | 1697 |
| 95 | LG:242966.4:2000SEP08 | g173.4258 | 1550 | 1642 |
| 95 | LG:242966.4:2000SEP08 | 477616R7 | 309 | 763 |
| 95 | LG:242966.4:2000SEP08 | 7766841J2 | 338 | 939 |
| 95 | LG:242966.4:2000SEP08 | 7763806H1 | 469 | 1087 |
| 95 | LG:242966.4:2000SEP08 | g2009268 | 496 | 708 |
| 95 | LG:242966.4:2000SEP08 | 867326H1 | 504 | 762 |
| 95 | LG:242966.4:2000SEP08 | 2180603F6 | 515 | 955 |
| 95 | LG:242966.4:2000SEP08 | 2180603H1 | 515 | 800 |
| 95 | LG:242966.4:2000SEP08 | 3344274H1 | 566 | 824 |
| 95 | LG:242966.4:2000SEP08 | 7262796H1 | 1 | 524 |
| 95 | LG:242966.4:2000SEP08 | 7633839J1 | 55 | 559 |
| 95 | LG:242966.4:2000SEP08 | 6699726H1 | 189 | 550 |
| 95 | LG:242966.4:2000SEP08 | 477616H1 | 309 | 570 |
| 95 | LG:242966.4:2000SEP08 | 6420668H1 | 214 | 388 |
| 95 | LG:242966.4:2000SEP08 | 2159734H1 | 277 | 500 |
| 95 | LG:242966.4:2000SEP08 | 784416H1 | 286 | 603 |
| 95 | LG:242966.4:2000SEP08 | 7677844H1 | 1485 | 1957 |
| 95 | LG:242966.4:2000SEP08 | 1403054H1 | 1479 | 1725 |
| 95 | LG:242966.4:2000SEP08 | 1403054F6 | 1479 | 1859 |
| 95 | LG:242966.4:2000SEP08 | 6769563J1 | 1497 | 1981 |
| 95 | LG:242966.4:2000SEP08 | 2490413H1 | 1506 | 1745 |
| 95 | LG:242966.4:2000SEP08 | 2180603T6 | 1509 | 1931 |
| 95 | LG:242966.4:2000SEP08 | 5817345H1 | 1522 | 1748 |
| 95 | LG:242966.4:2000SEP08 | 1475794R6 | 1281 | 1636 |
| 95 | LG:242966.4:2000SEP08 | 6352941F7 | 1403 | 1879 |
| 95 | LG:242966.4:2000SEP08 | 1475794H1 | 1281 | 1459 |
| 95 | LG:242966.4:2000SEP08 | 3016785H1 | 1414 | 1708 |
| 95 | LG:242966.4:2000SEP08 | 2489244T6 | 1290 | 1789 |
| 95 | LG:242966.4:2000SEP08 | 756332H1 | 1301 | 1559 |
| 95 | LG:242966.4:2000SEP08 | 7766841H1 | 1308 | 1587 |
| 95 | LG:242966.4:2000SEP08 | 1543702H1 | 1308 | 1491 |
| 95 | LG:242966.4:2000SEP08 | 47761617 | 1357 | 1905 |
| 95 | LG:242966.4:2000SEP08 | 1475794T6 | 1371 | 1921 |
| 95 | LG:242966.4:2000SEP08 | 2760146H1 | 1376 | 1648 |
| 95 | LG:242966.4:2000SEP08 | 6352941F8 | 1417 | 2023 |
| 95 | LG:242966.4:2000SEP08 | 6512741H1 | 1446 | 1589 |
| 95 | LG:242966.4:2000SEP08 | 7696070H1 | 1376 | 1565 |
| 95 | LG:242966.4:2000SEP08 | 7035051H1 | 1391 | 1964 |
| 95 | LG:242966.4:2000SEP08 | 2491496H1 | 1960 | 2026 |
| 95 | LG:242966.4:2000SEP08 | 2891711H1 | 613 | 884 |
| 95 | LG:242966.4:2000SEP08 | 2597301H1 | 1246 | 1515 |
| 95 | LG:242966.4:2000SEP08 | 3223664H1 | 632 | 893 |
| 95 | LG:242966.4:2000SEP08 | 6937623H1 | 757 | 1032 |
| 95 | LG:242966.4:2000SEP08 | 6300077H1 | 835 | 1118 |
| 95 | LG:242966.4:2000SEP08 | 2860363H1 | 881 | 1149 |
| 95 | LG:242966.4:2000SEP08 | 7763806J1 | 887 | 1510 |
| 95 | LG:242966.4:2000SEP08 | 2519487H1 | 945 | 1116 |
| 95 | LG:242966.4:2000SEP08 | 1572340H1 | 1134 | 1316 |
| 95 | LG:242966.4:2000SEP08 | 1507445H1 | 1146 | 1318 |
| 95 | LG:242966.4:2000SEP08 4635969H1 | 1184 | 1419 | |
| 95 | LG:242966.4:2000SEP08 | 2597301F6 | 1246 | 1631 |
| 95 | LG:242966.4:2000SEP08 | 4599922H1 | 1564 | 1843 |
| 95 | LG:242966.4:2000SEP08 | g5527462 | 1579 | 1973 |
| 95 | LG:242966.4:2000SEP08 | g4875402 | 1581 | 1981 |
| 95 | LG:242966.4:2000SEP08 | g3329924 | 1584 | 1973 |
| 95 | LG:242966.4:2000SEP08 | g5527471 | 1585 | 1973 |
| 95 | LG:242966.4:2000SEP08 | 7128343H2 | 1597 | 1970 |
| 96 | LG:217814.1:2000SEP08 | 2203116F6 | 1355 | 1841 |
| 96 | LG:217814.1:2000SEP08 | 2203116T6 | 1344 | 1838 |
| 96 | LG:217814.1:2000SEP08 | 6803273H1 | 1060 | 1530 |
| 96 | LG:217814.1:2000SEP08 | g1147087 | 1022 | 1386 |
| 96 | LG:217814.1:2000SEP08 | g2563845 | 1406 | 1815 |
| 96 | LG:217814.1:2000SEP08 | g2590584 | 1415 | 1826 |
| 96 | LG:217814.1:2000SEP08 | 2203116H1 | 1355 | 1611 |
| 96 | LG:217814.1:2000SEP08 | 2203035H1 | 1355 | 1590 |
| 96 | LG:217814.1:2000SEP08 | g1324443 | 1621 | 1842 |
| 96 | LG:217814.1:2000SEP08 | g3245150 | 1601 | 2005 |
| 96 | LG:217814.1:2000SEP08 | g4083193 | 1423 | 1820 |
| 96 | LG:217814.1:2000SEP08 | 6900096F9 | 1 | 378 |
| 96 | LG:217814.1:2000SEP08 | 6900096H1 | 1 | 449 |
| 96 | LG:217814.1:2000SEP08 | g4187748 | 1711 | 1784 |
| 96 | LG:217814.1:2000SEP08 | 7283212H1 | 175 | 586 |
| 96 | LG:217814.1:2000SEP08 | 3348991H1 | 239 | 394 |
| 96 | LG:217814.1:2000SEP08 | 260602H1 | 254 | 571 |
| 96 | LG:217814.1:2000SEP08 | 7720133H1 | 69 | 573 |
| 96 | LG:217814.1:2000SEP08 | 7285913H1 | 746 | 1289 |
| 96 | LG:217814.1:2000SEP08 | 7720133J1 | 424 | 968 |
| 97 | LG:476452.1:2000SEP08 | 3953832H1 | 790 | 921 |
| 97 | LG:476452.1:2000SEP08 | 6268518T8 | 564 | 1166 |
| 97 | LG:476452.1:2000SEP08 | 5911995T6 | 566 | 1106 |
| 97 | LG:476452.1:2000SEP08 | 5913343T9 | 598 | 1054 |
| 97 | LG:476452.1:2000SEP08 | 5911995T9 | 541 | 1080 |
| 97 | LG:476452.1:2000SEP08 | 6270538T8 | 562 | 1131 |
| 97 | LG:476452.1:2000SEP08 | 6268518F8 | 1 | 642 |
| 97 | LG:476452.1:2000SEP08 | 6270538H2 | 46 | 481 |
| 97 | LG:476452.1:2000SEP08 | 6270538F8 | 46 | 443 |
| 97 | LG:476452.1:2000SEP08 | 5913343F6 | 46 | 346 |
| 97 | LG:476452.1:2000SEP08 | 5913343H1 | 46 | 293 |
| 97 | LG:476452.1:2000SEP08 | 5911995F8 | 24 | 530 |
| 97 | LG:476452.1:2000SEP08 | 5911995H1 | 24 | 305 |
| 97 | LG:476452.1:2000SEP08 | 5913343F8 | 46 | 581 |
| 97 | LG:476452.1:2000SEP08 | 6268518H1 | 46 | 562 |
| 97 | LG:476452.1:2000SEP08 | 5911995F6 | 24 | 346 |
| 98 | LG:1100657.1:2000SEP08 | 6795168F8 | 1 | 509 |
| 98 | LG:1100657.1:2000SEP08 | 6797477H1 | 7 | 464 |
| 98 | LG:1100657.1:2000SEP08 | 6798453T8 | 217 | 481 |
| 98 | LG:1100657.1:2000SEP08 | 6793139H1 | 270 | 588 |
| 98 | LG:1100657.1:2000SEP08 | 679313918 | 270 | 486 |
| 98 | LG:1100657.1:2000SEP08 | 6793139F8 | 270 | 588 |
| 98 | LG:1100657.1:2000SEP08 | 6798453H1 | 1 | 327 |
| 98 | LG:1100657.1:2000SEP08 | 6795168H1 | 1 | 311 |
| 98 | LG:1100657.1:2000SEP08 | 6798453F8 | 1579 | |
| 98 | LG:1100657.1:2000SEP08 | 6797477T8 | 7 | 485 |
| 98 | LG:1100657.1:2000SEP08 | 6797477F8 | 7 | 544 |
| 99 | LG:1132418.2:2000SEP08 | 6615330H1 | 1 | 440 |
| 100 | LG:1098570.1:2000SEP08 | 6792174F8 | 1 | 574 |
| 100 | LG:1098570.1:2000SEP08 | 6792174T8 | 2 | 612 |
| 100 | LG:1098570.1:2000SEP08 | 6792174H1 | 1 | 566 |
| 101 | LG:1097987.1:2000SEP08 | 1445774F7 | 415 | 986 |
| 101 | LG:1097987.1:2000SEP08 | 2268189R6 | 132 | 526 |
| 101 | LG:1097987.1:2000SEP08 | 7680579H1 | 1 | 617 |
| 101 | LG:1097987.1:2000SEP08 | 2268189T6 | 481 | 1032 |
| 101 | LG:1097987.1:2000SEP08 | 2268189H1 | 132 | 378 |
| 101 | LG:1097987.1:2000SEP08 | 144577417 | 515 | 966 |
| 101 | LG:1097987.1:2000SEP08 | 368103H1 | 667 | 910 |
| 101 | LG:1097987.1:2000SEP08 | 7680579J1 | 386 | 930 |
| 101 | LG:1097987.1:2000SEP08 | 1445774H1 | 409 | 654 |
| 101 | LG:1097987.1:2000SEP08 | g2328398 | 755 | 1076 |
| 101 | LG:1097987.1:2000SEP08 | 2322571H1 | 837 | 1070 |
| 102 | LG:337818.2:2000SEP08 | 6883053H1 | 527 | 1106 |
| 102 | LG:337818.2:2000SEP08 | 6576957H1 | 1907 | 2455 |
| 102 | LG:337818.2:2000SEP08 | 3254138H1 | 1908 | 2115 |
| 102 | LG:337818.2:2000SEP08 | 5946104H1 | 1910 | 2145 |
| 102 | LG:337818.2:2000SEP08 | 7748294H1 | 1911 | 2424 |
| 102 | LG:337818.2:2000SEP08 | 5038396H1 | 1888 | 2146 |
| 102 | LG:337818.2:2000SEP08 | 6820526H1 | 422 | 747 |
| 102 | LG:337818.2:2000SEP08 | 6820526J1 | 422 | 747 |
| 102 | LG:337818.2:2000SEP08 | 7710749J1 | 46 | 700 |
| 102 | LG:337818.2:2000SEP08 | 4516041H1 | 1817 | 2066 |
| 102 | LG:337818.2:2000SEP08 | 4180584H1 | 1872 | 2122 |
| 102 | LG:337818.2:2000SEP08 | g1968950 | 1886 | 2368 |
| 102 | LG:337818.2:2000SEP08 | 1272843H1 | 1 | 245 |
| 102 | LG:337818.2:2000SEP08 | 1274319H1 | 21 | 218 |
| 102 | LG:337818.2:2000SEP08 | 2598183H1 | 24 | 263 |
| 102 | LG:337818.2:2000SEP08 | 3256051H1 | 25 | 268 |
| 102 | LG:337818.2:2000SEP08 | 2598183F6 | 31 | 607 |
| 102 | LG:337818.2:2000SEP08 | 6826541J1 | 33 | 663 |
| 102 | LG:337818.2:2000SEP08 | 6823903H1 | 34 | 188 |
| 102 | LG:337818.2:2000SEP08 | 7218761H1 | 35 | 559 |
| 102 | LG:337818.2:2000SEP08 | 7719403H1 | 46 | 603 |
| 102 | LG:337818.2:2000SEP08 | 6786878H2 | 43 | 184 |
| 102 | LG:337818.2:2000SEP08 | 1889107H1 | 1542 | 1812 |
| 102 | LG:337818.2:2000SEP08 | 1889107F6 | 1542 | 1892 |
| 102 | LG:337818.2:2000SEP08 | 6725031H1 | 1675 | 2299 |
| 102 | LG:337818.2:2000SEP08 | 6823952J1 | 1549 | 2162 |
| 102 | LG:337818.2:2000SEP08 | 5307668H1 | 1567 | 1691 |
| 102 | LG:337818.2:2000SEP08 | 5840503H2 | 1578 | 1835 |
| 102 | LG:337818.2:2000SEP08 | 4519262H1 | 2360 | 2599 |
| 102 | LG:337818.2:2000SEP08 | 1272843T6 | 2368 | 2562 |
| 102 | LG:337818.2:2000SEP08 | 6104909H1 | 2413 | 2604 |
| 102 | LG:337818.2:2000SEP08 | 5056523H1 | 2466 | 2604 |
| 102 | LG:337818.2:2000SEP08 | g5038163 | 2168 | 2608 |
| 102 | LG:337818.2:2000SEP08 | g3146568 | 2200 | 2604 |
| 102 | LG:337818.2:2000SEP08 | g2355286 | 2200 | 2589 |
| 102 | LG:337818.2:2000SEP08 | 1845337T6 | 2212 | 2566 |
| 102 | LG:337818.2:2000SEP08 | 1845337H1 | 2212 | 2433 |
| 102 | LG:337818.2:2000SEP08 | 1845337R6 | 2212 | 2562 |
| 102 | LG:337818.2:2000SEP08 | g4288591 | 2215 | 2599 |
| 102 | LG:337818.2:2000SEP08 | g5659132 | 2219 | 2551 |
| 102 | LG:337818.2:2000SEP08 | 913114H1 | 2228 | 2509 |
| 102 | LG:337818.2:2000SEP08 | g1960980 | 2253 | 2604 |
| 102 | LG:337818.2:2000SEP08 | g5887689 | 2244 | 2601 |
| 102 | LG:337818.2:2000SEP08 | 2700155F6 | 1922 | 2433 |
| 102 | LG:337818.2:2000SEP08 | 2700155H1 | 1922 | 2121 |
| 102 | LG:337818.2:2000SEP08 | 2061567T6 | 1951 | 2554 |
| 102 | LG:337818.2:2000SEP08 | 3365965H1 | 1983 | 2108 |
| 102 | LG:337818.2:2000SEP08 | g5591869 | 2006 | 2220 |
| 102 | LG:337818.2:2000SEP08 | 1749882T6 | 2039 | 2571 |
| 102 | LG:337818.2:2000SEP08 | 1752020H1 | 2049 | 2258 |
| 102 | LG:337818.2:2000SEP08 | 2700155T6 | 2063 | 2563 |
| 102 | LG:337818.2:2000SEP08 | 1889107T6 | 2099 | 2562 |
| 102 | LG:337818.2:2000SEP08 | 3236592H2 | 2125 | 2340 |
| 102 | LG:337818.2:2000SEP08 | 5291302H1 | 2152 | 2398 |
| 102 | LG:337818.2:2000SEP08 | g5838009 | 2250 | 2611 |
| 102 | LG:337818.2:2000SEP08 | 911966H1 | 2249 | 2503 |
| 102 | LG:337818.2:2000SEP08 | g5913083 | 2251 | 2604 |
| 102 | LG:337818.2:2000SEP08 | g3741346 | 2254 | 2611 |
| 102 | LG:337818.2:2000SEP08 | g3785022 | 2297 | 2602 |
| 102 | LG:337818.2:2000SEP08 | g2463906 | 2312 | 2607 |
| 102 | LG:337818.2:2000SEP08 | g4312734 | 2347 | 2599 |
| 102 | LG:337818.2:2000SEP08 | g2100983 | 2358 | 2591 |
| 102 | LG:337818.2:2000SEP08 | 1272843F1 | 1 | 331 |
| 102 | LG:337818.2:2000SEP08 | 1272843F6 | 1 | 593 |
| 102 | LG:337818.2:2000SEP08 | 2061567R6 | 1698 | 2177 |
| 102 | LG:337818.2:2000SEP08 | 2061567H1 | 1698 | 1970 |
| 102 | LG:337818.2:2000SEP08 | 4399331H1 | 1734 | 2000 |
| 102 | LG:337818.2:2000SEP08 | 4399135H1 | 1734 | 1991 |
| 102 | LG:337818.2:2000SEP08 | 2250985H1 | 1734 | 1964 |
| 102 | LG:337818.2:2000SEP08 | 1501428H1 | 1737 | 1930 |
| 102 | LG:337818.2:2000SEP08 | 6018675H1 | 1812 | 2378 |
| 102 | LG:337818.2:2000SEP08 | 7713423J1 | 1483 | 2070 |
| 102 | LG:337818.2:2000SEP08 | 794690H1 | 1496 | 1698 |
| 102 | LG:337818.2:2000SEP08 | 6576416H1 | 1513 | 1850 |
| 102 | LG:337818.2:2000SEP08 | 476160H1 | 1389 | 1655 |
| 102 | LG:337818.2:2000SEP08 | 6158833H1 | 1433 | 1523 |
| 102 | LG:337818.2:2000SEP08 | 4552488H1 | 958 | 1120 |
| 102 | LG:337818.2:2000SEP08 | 7219413H1 | 999 | 1496 |
| 102 | LG:337818.2:2000SEP08 | 4243388H1 | 919 | 1251 |
| 102 | LG:337818.2:2000SEP08 | g713204 | 1029 | 1191 |
| 102 | LG:337818.2:2000SEP08 | g1967839 | 1091 | 1429 |
| 102 | LG:337818.2:2000SEP08 | 7680157J1 | 1174 | 1775 |
| 102 | LG:337818.2:2000SEP08 | 7675304J1 | 1177 | 1790 |
| 102 | LG:337818.2:2000SEP08 | 7680157H1 | 956 | 1465 |
| 102 | LG:337818.2:2000SEP08 | 7710749H1 | 1261 | 1790 |
| 102 | LG:337818.2:2000SEP08 | 3737077H1 | 1295 | 1453 |
| 102 | LG:337818.2:2000SEP08 | 6326476H1 | 1313 | 1610 |
| 102 | LG:337818.2:2000SEP08 | g1968949 | 1345 | 1812 |
| 102 | LG:337818.2:2000SEP08 | g2229641 | 1360 | 1792 |
| 102 | LG:337818.2:2000SEP08 | 7713423H1 | 664 | 1234 |
| 102 | LG:337818.2:2000SEP08 | 7719403J1 | 528 | 1194 |
| 102 | LG:337818.2:2000SEP08 | 6826541H1 | 672 | 1240 |
| 102 | LG:337818.2:2000SEP08 | 7634622J1 | 718 | 1162 |
| 102 | LG:337818.2:2000SEP08 | 7634622H1 | 720 | 1162 |
| 102 | LG:337818.2:2000SEP08 | 3857549H1 | 797 | 1106 |
| 102 | LG:337818.2:2000SEP08 | 1749882H1 | 811 | 1085 |
| 102 | LG:337818.2:2000SEP08 | 1749882F6 | 811 | 1171 |
| 102 | LG:337818.2:2000SEP08 | 7675304H1 | 658 | 1121 |
| 102 | LG:337818.2:2000SEP08 | 6829715H1 | 860 | 1357 |
| 102 | LG:337818.2:2000SEP08 | 7746862H1 | 873 | 1313 |
| 102 | LG:337818.2:2000SEP08 | g1953371 | 885 | 986 |
| 102 | LG:337818.2:2000SEP08 | g1953334 | 885 | 1086 |
| 102 | LG:337818.2:2000SEP08 | 2271032R6 | 893 | 1370 |
| 102 | LG:337818.2:2000SEP08 | 2271032H1 | 893 | 1152 |
| 102 | LG:337818.2:2000SEP08 | 7128738H1 | 913 | 1433 |
| 103 | LG:1040582.1:2000SEP08 | 91260446 | 2 | 321 |
| 103 | LG:1040582.1:2000SEP08 | 6791379H1 | 1 | 402 |
| 103 | LG:1040582.1:2000SEP08 | g1614819 | 220 | 660 |
| 103 | LG:1040582.1:2000SEP08 | g1647514 | 249 | 548 |
| 104 | LG:1099122.1:2000SEP08 | 6798356T8 | 13 | 405 |
| 104 | LG:1099122.1:2000SEP08 | 6793934H1 | 2 | 425 |
| 104 | LG:1099122.1:2000SEP08 | 6798356H1 | 14 | 514 |
| 104 | LG:1099122.1:2000SEP08 | 6790886T8 | 1 | 416 |
| 104 | LG:1099122.1:2000SEP08 | 6790886F8 | 1 | 537 |
| 104 | LG:1099122.1:2000SEP08 | 595627H1 | 138 | 223 |
| 104 | LG:1099122.1:2000SEP08 | 6798356F8 | 14 | 518 |
| 104 | LG:1099122.1:2000SEP08 | 6790886H1 | 15 | 529 |
| 105 | LG:1327449.1:2000SEP08 | 2870587F6 | 1 | 500 |
| 105 | LG:1327449.1:2000SEP08 | 2870587H1 | 1 | 262 |
| 105 | LG:1327449.1:2000SEP08 | 2870587T6 | 233 | 564 |
| 105 | LG:1327449.1:2000SEP08 | 1692515H1 | 338 | 553 |
| 105 | LG:1327449.1:2000SEP08 | 2753173H1 | 418 | 595 |
| 105 | LG:1327449.1:2000SEP08 | 5754938H1 | 497 | 596 |
| 106 | LG:227933.5:2000SEP08 | 7701259J1 | 183 | 820 |
| 106 | LG:227933.5:2000SEP08 | g6402044 | 1 | 505 |
| 106 | LG:227933.5:2000SEP08 | 7701259H1 | 1 | 503 |
| 106 | LG:227933.5:2000SEP08 | 7700960H1 | 7 | 583 |
| 106 | LG:227933.5:2000SEP08 | 7700960J1 | 301 | 820 |
| 106 | LG:227933.5:2000SEP08 | 7242063H1 | 113 | 702 |
| 106 | LG:227933.5:2000SEP08 | 270716H1 | 1017 | 1350 |
| 106 | LG:227933.5:2000SEP08 | 3337660H1 | 1114 | 1369 |
| 106 | LG:227933.5:2000SEP08 | 3337660F6 | 1114 | 1457 |
| 106 | LG:227933.5:2000SEP08 | 008680H1 | 1221 | 1504 |
| 106 | LG:227933.5:2000SEP08 | 4163591H1 | 1276 | 1576 |
| 106 | LG:227933.5:2000SEP08 | 7583129H1 | 303 | 862 |
| 106 | LG:227933.5:2000SEP08 | 3825814H1 | 305 | 598 |
| 106 | LG:227933.5:2000SEP08 | 3825862H1 | 306 | 589 |
| 106 | LG:227933.5:2000SEP08 | 6859841H1 | 337 | 895 |
| 106 | LG:227933.5:2000SEP08 | 3618330H1 | 468 | 766 |
| 106 | LG:227933.5:2000SEP08 | 2984273H1 | 469 | 731 |
| 106 | LG:227933.5:2000SEP08 | 6476227H1 | 634 | 1170 |
| 106 | LG:227933.5:2000SEP08 | 269722H1 | 837 | 1213 |
| 106 | LG:227933.5:2000SEP08 | 269722R6 | 838 | 1281 |
| 106 | LG:227933.5:2000SEP08 | 5090567H1 | 837 | 1127 |
| 106 | LG:227933.5:2000SEP08 | 269722R1 | 838 | 1344 |
| 106 | LG:227933.5:2000SEP08 | 5861888H1 | 869 | 1118 |
| 107 | LG:1043709.2:2000SEP08 | 6791130H1 | 1 | 431 |
| 107 | LG:1043709.2:2000SEP08 | 6791130T8 | 151 | 780 |
| 108 | LG:1099871.1:2000SEP08 | 6793081T8 | 1 | 560 |
| 108 | LG:1099871.1:2000SEP08 | 7236484H1 | 503 | 673 |
| 108 | LG:1099871.1:2000SEP08 | 6793081F8 | 1 | 667 |
| 108 | LG:1099871.1:2000SEP08 | g1626426 | 479 | 656 |
| 108 | LG:1099871.1:2000SEP08 | 6791420T8 | 1 | 556 |
| 108 | LG:1099871.1:2000SEP08 | 6793081H1 | 1 | 437 |
| 108 | LG:1099871.1:2000SEP08 | 6791420F8 | 1 284 | |
| 108 | LG:1099871.1:2000SEP08 | 6791420H1 | 1 | 207 |
| 109 | LG:1399139.4:2000SEP08 | g2167704 | 342 | 607 |
| 109 | LG:1399139.4:2000SEP08 | g2167287 | 539 | 601 |
| 109 | LG:1399139.4:2000SEP08 | g5446758 | 192 | 601 |
| 109 | LG:1399139.4:2000SEP08 | g3076369 | 397 | 558 |
| 109 | LG:1399139.4:2000SEP08 | 5543619T7 | 1 | 497 |
| 109 | LG:1399139.4:2000SEP08 | 3210126H1 | 309 | 408 |
| 109 | LG:1399139.4:2000SEP08 | 6767602H1 | 342 | 783 |
| 109 | LG:1399139.4:2000SEP08 | g2113065 | 354 | 676 |
| 109 | LG:1399139.4:2000SEP08 | 6215226H1 | 221 | 670 |
| 110 | LG:236386.1:2000SEP08 | 6317150H1 | 3421 | 3711 |
| 110 | LG:236386.1:2000SEP08 | 6317128H1 | 3421 | 3548 |
| 110 | LG:236386.1:2000SEP08 | 6346421H1 | 3421 | 3702 |
| 110 | LG:236386.1:2000SEP08 | 2661806T6 | 3447 | 3843 |
| 110 | LG:236386.1:2000SEP08 | g3429071 | 3462 | 3880 |
| 110 | LG:236386.1:2000SEP08 | g5810032 | 3472 | 3886 |
| 110 | LG:236386.1:2000SEP08 | 7255223H1 | 3492 | 3875 |
| 110 | LG:236386.1:2000SEP08 | g2237335 | 3502 | 3880 |
| 110 | LG:236386.1:2000SEP08 | 2878117H1 | 3505 | 3776 |
| 110 | LG:236386.1:2000SEP08 | g1400734 | 3511 | 3875 |
| 110 | LG:236386.1:2000SEP08 | 6325947H1 | 3421 | 3714 |
| 110 | LG:236386.1:2000SEP08 | 5852153H1 | 3405 | 3669 |
| 110 | LG:236386.1:2000SEP08 | 4333836H1 | 3394 | 3671 |
| 110 | LG:236386.1:2000SEP08 | 840648H1 | 3394 | 3640 |
| 110 | LG:236386.1:2000SEP08 | 2615527H1 | 2645 | 2897 |
| 110 | LG:236386.1:2000SEP08 | 1438880H1 | 2708 | 2980 |
| 110 | LG:236386.1:2000SEP08 | 1438876H1 | 2708 | 2978 |
| 110 | LG:236386.1:2000SEP08 | 1438876F1 | 2708 | 3074 |
| 110 | LG:236386.1:2000SEP08 | 684595H1 | 2952 | 3200 |
| 110 | LG:236386.1:2000SEP08 | 2258046H1 | 2738 | 2973 |
| 110 | LG:236386.1:2000SEP08 | 7735093H1 | 2980 | 3603 |
| 110 | LG:236386.1:2000SEP08 | 6479471H1 | 2806 | 3340 |
| 110 | LG:236386.1:2000SEP08 | 7054594H1 | 2816 | 3383 |
| 110 | LG:236386.1:2000SEP08 | 6722223H1 | 3022 | 3195 |
| 110 | LG:236386.1:2000SEP08 | 5274874H | 12848 | 3075 |
| 110 | LG:236386.1:2000SEP08 | 2857322H1 | 2920 | 3177 |
| 110 | LG:236386.1:2000SEP08 | 792748H1 | 2924 | 3151 |
| 110 | LG:236386.1:2000SEP08 | 793130H1 | 2925 | 3131 |
| 110 | LG:236386.1:2000SEP08 | 792748R1 | 2925 | 3509 |
| 110 | LG:236386.1:2000SEP08 | 7159471H1 | 2934 | 3484 |
| 110 | LG:236386.1:2000SEP08 | 1541872H1 | 2951 | 3157 |
| 110 | LG:236386.1:2000SEP08 | 6559394H1 | 1836 | 2450 |
| 110 | LG:236386.1:2000SEP08 | 6553230H1 | 1836 | 2188 |
| 110 | LG:236386.1:2000SEP08 | 3382113H1 | 1906 | 2113 |
| 110 | LG:236386.1:2000SEP08 | 2272356H1 | 1647 | 1915 |
| 110 | LG:236386.1:2000SEP08 | 2661806H1 | 2112 | 2383 |
| 110 | LG:236386.1:2000SEP08 | 2661806F6 | 2112 | 2553 |
| 110 | LG:236386.1:2000SEP08 | g6476309 | 2172 | 2528 |
| 110 | LG:236386.1:2000SEP08 | 2627073H1 | 2183 | 2413 |
| 110 | LG:236386.1:2000SEP08 | 2627315H1 | 2183 | 2411 |
| 110 | LG:236386.1:2000SEP08 | 3901711H1 | 2270 | 2513 |
| 110 | LG:236386.1:2000SEP08 | 5763849H1 | 2373 | 2890 |
| 110 | LG:236386.1:2000SEP08 | 7256511H1 | 2420 | 2921 |
| 110 | LG:236386.1:2000SEP08 | 3572311H1 | 2509 | 2721 |
| 110 | LG:236386.1:2000SEP08 | 3572311F6 | 2509 | 3086 |
| 110 | LG:236386.1:2000SEP08 | 7336064H1 | 2549 | 2991 |
| 110 | LG:236386.1:2000SEP08 | 2272356T6 | 2588 | 3010 |
| 110 | LG:236386.1:2000SEP08 | 685902H1 | 2627 | 2848 |
| 110 | LG:236386.1:2000SEP08 | 6831490J1 | 496 | 688 |
| 110 | LG:236386.1:2000SEP08 | 6831490H1 | 496 | 688 |
| 110 | LG:236386.1:2000SEP08 | 7738646H1 | 755 | 1252 |
| 110 | LG:236386.1:2000SEP08 | 6938224H1 | 973 | 1366 |
| 110 | LG:236386.1:2000SEP08 | 7267489H1 | 1082 | 1603 |
| 110 | LG:236386.1:2000SEP08 | g3888759 | 1148 | 1514 |
| 110 | LG:236386.1:2000SEP08 | 6454789H1 | 1317 | 1820 |
| 110 | LG:236386.1:2000SEP08 | 684735H1 | 1380 | 1626 |
| 110 | LG:236386.1:2000SEP08 | 7649660H2 | 1420 | 2039 |
| 110 | LG:236386.1:2000SEP08 | 6952285H1 | 1506 | 2072 |
| 110 | LG:236386.1:2000SEP08 | 4458494F6 | 1519 | 1967 |
| 110 | LG:236386.1:2000SEP08 | 4458494H1 | 1520 | 1755 |
| 110 | LG:236386.1:2000SEP08 | 7255931H2 | 1596 | 1777 |
| 110 | LG:236386.1:2000SEP08 | 6909665J1 | 1633 | 2177 |
| 110 | LG:236386.1:2000SEP08 | 2272356R6 | 1647 | 1966 |
| 110 | LG:236386.1:2000SEP08 | 4181419H1 | 1 | 220 |
| 110 | LG:236386.1:2000SEP08 | 6779195J1 | 119 | 758 |
| 110 | LG:236386.1:2000SEP08 | 113399R6 | 483 | 847 |
| 110 | LG:236386.1:2000SEP08 | 5104505H1 | 3515 | 3738 |
| 110 | LG:236386.1:2000SEP08 | g4081742 | 3517 | 3883 |
| 110 | LG:236386.1:2000SEP08 | 1452312T6 | 3521 | 3836 |
| 110 | LG:236386.1:2000SEP08 | g898312 | 3538 | 3878 |
| 110 | LG:236386.1:2000SEP08 | 6499719H1 | 3537 | 3869 |
| 110 | LG:236386.1:2000SEP08 | g4081564 | 3538 | 3883 |
| 110 | LG:236386.1:2000SEP08 | g2335900 | 3571 | 3880 |
| 110 | LG:236386.1:2000SEP08 | g6451467 | 3573 | 3875 |
| 110 | LG:236386.1:2000SEP08 | g1521304 3576 | 3891 | |
| 110 | LG:236386.1:2000SEP08 | p4534027 | 3577 | 3883 |
| 110 | LG:236386.1:2000SEP08 | 5790863H 1 | 3580 | 3863 |
| 110 | LG:236386.1:2000SEP08 5787849H1 | 3580 | 3875 | |
| 110 | LG:236386.1:2000SEP08 5789451H1 | 3580 | 3858 | |
| 110 | LG:236386.1:2000SEP08 | p5528373 | 3592 | 3880 |
| 110 | LG:236386.1:2000SEP08 | p1516463 | 3595 | 3891 |
| 110 | LG:236386.1:2000SEP08 | 95912966 | 3628 | 3880 |
| 110 | LG:236386.1:2000SEP08 | 344685H1 | 3641 | 3882 |
| 110 | LG:236386.1:2000SEP08 | 389997H1 | 3639 | 3875 |
| 110 | LG:236386.1:2000SEP08 | 6357624H 1 | 3650 | 3882 |
| 110 | LG:236386.1:2000SEP08 | g3961665 | 3652 | 3880 |
| 110 | LG:236386.1:2000SEP08 | p6477150 | 3654 | 3885 |
| 110 | LG:236386.1:2000SEP08 | 1689958F6 | 3661 | 3883 |
| 110 | LG:236386.1:2000SEP08 | 1689958H1 | 3661 | 3867 |
| 110 | LG:236386.1:2000SEP08 | 1 689958T6 | 3666 | 3840 |
| 110 | LG:236386.1:2000SEP08 | 1702166T6 | 3684 | 3826 |
| 110 | LG:236386.1:2000SEP08 | 357231 1T6 | 3705 | 3832 |
| 110 | LG:236386.1:2000SEP08 | p4649451 | 3752 | 3875 |
| 110 | LG:236386.1:2000SEP08 | 2599007H 1 | 3298 | 3562 |
| 110 | LG:236386.1:2000SEP08 | 1320150H1 | 3124 | 3347 |
| 110 | LG:236386.1:2000SEP08 | 4897563H1 | 3126 | 3401 |
| 110 | LG:236386.1:2000SEP08 | 5379052H1 | 3134 | 3346 |
| 110 | LG:236386.1:2000SEP08 | 3406784H1 | 3142 | 3390 |
| 110 | LG:236386.1:2000SEP08 | 2527855H1 | 3172 | 3504 |
| 110 | LG:236386.1:2000SEP08 | g1521303 | 3191 | 3623 |
| 110 | LG:236386.1:2000SEP08 | p1517127 | 3191 | 3666 |
| 110 | LG:236386.1:2000SEP08 | 709518H1 | 3205 | 3428 |
| 110 | LG:236386.1:2000SEP08 | 2414749F6 | 3208 | 3714 |
| 110 | LG:236386.1:2000SEP08 | 3776430H1 | 3207 | 3498 |
| 110 | LG:236386.1:2000SEP08 | 2414751H1 | 3208 | 3467 |
| 110 | LG:236386.1:2000SEP08 | 2414483H1 | 3208 | 3432 |
| 110 | LG:236386.1:2000SEP08 | 7738646J1 | 3262 | 3865 |
| 110 | LG:236386.1:2000SEP08 | p898311 | 3269 | 3438 |
| 110 | LG:236386.1:2000SEP08 | 1452312F1 | 3275 | 3796 |
| 110 | LG:236386.1:2000SEP08 | 1452312F6 | 3275 | 3702 |
| 110 | LG:236386.1:2000SEP08 | 1452312H1 | 3275 | 3534 |
| 110 | LG:236386.1:2000SEP08 | 3011048H1 | 3326 | 3611 |
| 110 | LG:236386.1:2000SEP08 | 4562117H1 | 3334 | 3584 |
| 110 | LG:236386.1:2000SEP08 | 4563263H1 | 3335 | 3606 |
| 110 | LG:236386.1:2000SEP08 | 2806050H1 | 3027 | 3331 |
| 110 | LG:236386.1:2000SEP08 | 7649660J2 | 3034 | 3668 |
| 110 | LG:236386.1:2000SEP08 | 1702166F6 | 3047 | 3541 |
| 110 | LG:236386.1:2000SEP08 | 1702166H1 | 3047 | 3258 |
| 110 | LG:236386.1:2000SEP08 | 4980587H1 | 3060 | 3313 |
| 110 | LG:236386.1:2000SEP08 | 6909665H1 | 3076 | 3593 |
| 110 | LG:236386.1:2000SEP08 | 6074761H1 | 3079 | 3376 |
| 110 | LG:236386.1:2000SEP08 | 4372755H1 | 3078 | 3366 |
| 110 | LG:236386.1:2000SEP08 | 872814H1 | 3082 | 3273 |
| 110 | LG:236386.1:2000SEP08 | 5438843H1 | 3097 | 3383 |
| 110 | LG:236386.1:2000SEP08 | 4880465H1 | 3100 | 3335 |
| 110 | LG:236386.1:2000SEP08 | 2658395H1 | 3974 | 4210 |
| 110 | LG:236386.1:2000SEP08 | 4099042H2 | 3777 | 3887 |
| 110 | LG:236386.1:2000SEP08 | 1243554H1 | 3777 | 3883 |
| 110 | LG:236386.1:2000SEP08 | 7645333H1 | 3782 | 4137 |
| 110 | LG:236386.1:2000SEP08 | 7645333J1 | 3782 | 4131 |
| 110 | LG:236386.1:2000SEP08 | g4325490 | 3795 | 3875 |
| 110 | LG:236386.1:2000SEP08 | 700495H1 | 3961 | 4212 |
| 110 | LG:236386.1:2000SEP08 | 2623608H1 | 3349 | 3576 |
| 110 | LG:236386.1:2000SEP08 | 840648R1 | 3394 | 3875 |
| 111 | LG:1015157.1:2000SEP08 | 6795378F8 | 1 | 638 |
| 111 | LG:1015157.1:20003EP08 | 6795378H1 | 2 | 438 |
| 111 | LG:1015157.1:2000SEP08 | 6795378T8 | 175 | 658 |
| 112 | LG:1065433.1:2000SEP08 | 4023626H1 | 701 | 980 |
| 112 | LG:1065433.1:2000SEP08 | 4216384H1 | 563 | 832 |
| 112 | LG:1065433.1:2000SEP08 | 3449946H1 | 1 | 233 |
| 112 | LG:1065433.1:2000SEP08 | 5133790T9 | 317 | 911 |
| 112 | LG:1065433.1:2000SEP08 | 421638.4F6 | 563 | 1029 |
| 112 | LG:1065433.1:2000SEP08 | 3449946R6 | 1 | 580 |
| 113 | LG:236992.4:2000SEP08 | 2023720H1 | 380 | 603 |
| 113 | LG:236992.4:2000SEP08 | 5711092T6 | 425 | 897 |
| 113 | LG:236992.4:2000SEP08 | 2023720T6 | 621 | 988 |
| 113 | LG:236992.4:2000SEP08 | 1957414H1 | 645 | 918 |
| 113 | LG:236992.4:2000SEP08 | 3691485H1 | 302 | 574 |
| 113 | LG:236992.4:2000SEP08 | 7582909H 1 | 1 | 443 |
| 113 | LG:236992.4:2000SEP08 | 571 1092F6 | 1 | 595 - |
| 113 | LG:236992.4:2000SEP08 | 5711092H1 | 1 | 277 |
| 113 | LG:236992.4:2000SEP08 | 3366992F7 | 9 | 280 |
| 113 | LG:236992.4:2000SEP08 | 3366992H1 | 9 | 276 |
| 113 | LG:236992.4:2000SEP08 | 4415026H1 | 10 | 250 |
| 113 | LG:236992.4:2000SEP08 | 1672338H1 | 18 | 238 |
| 113 | LG:236992.4:2000SEP08 | 2023720F6 | 379 | 746 |
| 114 | LG:1071124.1:20003EP08 | 6182057F7 | 342 | 928 |
| 114 | LG:1071124.1:2000SEP08 | g3092207 | 896 | 1332 |
| 114 | LG:1071124.1:2000SEP08 | 2950882H1 | 1035 | 1171 |
| 114 | LG:1071124.1:2000SEP08 | g3739050 | 494 | 820 |
| 114 | LG:1071124.1:2000SEP08 | 6880110H1 | 1 | 365 |
| 114 | LG:1071124.1:2000SEP08 | 6273782F8 | 656 | 1305 |
| 114 | LG:1071124.1:2000SEP08 | g1880374 | 545 | 648 |
| 114 | LG:1071124.1:2000SEP08 | 6182057H1 | 668 | 928 |
| 114 | LG:1071124.1:2000SEP08 | 6272957T8 | 583 | 1276 |
| 114 | LG:1071124.1:2000SEP08 | g1639276 | 582 | 840 |
| 114 | LG:1071124.1:2000SEP08 | 6272957H1 | 755 | 1305 |
| 114 | LG:1071124.1:2000SEP08 | 4290705H1 | 229 | 481 |
| 114 | LG:1071124.1:2000SEP08 | 494966H1 | 245 | 471 |
| 114 | LG:1071124.1:2000SEP08 | 4099889H1 | 225 | 479 |
| 114 | LG:1071124.1:2000SEP08 | 4290705F6 | 228 | 609 |
| 114 | LG:1071124.1:2000SEP08 | 1676346F6 | 213 | 459 |
| 114 | LG:1071124.1:2000SEP08 | 7196034H1 | 1 | 453 |
| 114 | LG:1071124.1:2000SEP08 | 1676346H1 | 213 | 438 |
| 115 | LG:206425.2:2000SEP08 | 5523393H1 | 1 | 255 |
| 115 | LG:206425.2:2000SEP08 | g6074738 | 986 | 1391 |
| 115 | LG:206425.2:2000SEP08 | 5523393R6 | 17 | 547 |
| 115 | LG:206425.2:2000SEP08 | 6218914H1 | 305 | 636 |
| 115 | LG:206425.2:2000SEP08 | 6218993H1 | 312 | 629 |
| 115 | LG:206425.2:2000SEP08 | 7382646H1 | 439 | 1050 |
| 115 | LG:206425.2:2000SEP08 | 6447108H1 | 451 | 1013 |
| 115 | LG:206425.2:2000SEP08 | g1969689 | 578 | 969 |
| 115 | LG:206425.2:2000SEP08 | 4603009H1 | 633 | 917 |
| 115 | LG:206425.2:2000SEP08 | 4594650F6 | 639 | 1130 |
| 115 | LG:206425.2:2000SEP08 | 6757573J1 | 667 | 898 |
| 115 | LG:206425.2:2000SEP08 | 5995878H1 | 753 | 947 |
| 115 | LG:206425.2:2000SEP08 | 5432648R7 | 754 | 1369 |
| 115 | LG:206425.2:2000SEP08 | 579733H1 | 1565 | 1802 |
| 115 | LG:206425.2:2000SEP08 | 3169352H1 | 1439 | 1735 |
| 115 | LG:206425.2:2000SEP08 | g2335907 | 1450 | 1842 |
| 115 | LG:206425.2:2000SEP08 | 579296H1 | 1565 | 1820 |
| 115 | LG:206425.2:2000SEP08 | g1442259 | 1258 | 1541 |
| 115 | LG:206425.2:2000SEP08 | 1959829H1 | 1404 | 1655 |
| 115 | LG:206425.2:2000SEP08 | g3918913 | 1409 | 1843 |
| 115 | LG:206425.2:2000SEP08 | 3169352F6 | 1439 | 1762 |
| 115 | LG:206425.2:2000SEP08 | 3163810H1 | 1046 | 1317 |
| 115 | LG:206425.2:2000SEP08 | 5523393F6 | 1 | 543 |
| 115 | LG:206425.2:2000SEP08 | 7267514H1 | 1616 | 1803 |
| 115 | LG:206425.2:2000SEP08 | 011293H1 | 1690 | 1808 |
| 115 | LG:206425.2:2000SEP08 | 7619271H1 | 1693 | 2265 |
| 115 | LG:206425.2:2000SEP08 | g4006331 | 1588 | 1996 |
| 115 | LG:206425.2:2000SEP08 | 013319H1 | 2244 | 2377 |
| 115 | LG:206425.2:2000SEP08 | 393646R6 | 2245 | 2410 |
| 115 | LG:206425.2:2000SEP08 | 4535173H1 | 2076 | 2163 |
| 115 | LG:206425.2:2000SEP08 | 6757573H1 | 2222 | 2379 |
| 115 | LG:206425.2:2000SEP08 | g1733736 | 1898 | 2066 |
| 115 | LG:206425.2:2000SEP08 | 6755190J1 | 1937 | 2000 |
| 115 | LG:206425.2:2000SEP08 | 815254H1 | 1817 | 2083 |
| 115 | LG:206425.2:2000SEP08 | 6721404H1 | 1890 | 2000 |
| 115 | LG:206425.2:2000SEP08 | 815254R1 | 1817 | 2378 |
| 116 | LG:885747.2:2000SEP08 | 6072957T8 | 1 | 599 |
| 117 | LG:1140501.1:2000SEP08 | 2266814H1 | 1037 | 1269 |
| 117 | LG:1140501.1:2000SEP08 | 1554513H1 | 1046 | 1239 |
| 117 | LG:1140501.1:2000SEP08 | 235788H1 | 1048 | 1244 |
| 117 | LG:1140501.1:2000SEP08 | 1522424H1 | 1058 | 1233 |
| 117 | LG:1140501.1:2000SEP08 | 1475643H1 | 1116 | 1328 |
| 117 | LG:1140501.1:2000SEP08 | 1757269H1 | 1118 | 1368 |
| 117 | LG:1140501.1:2000SEP08 | 2210487H1 | 1134 | 1370 |
| 117 | LG:1140501.1:2000SEP08 | 3905369H1 | 1165 | 1428 |
| 117 | LG:1140501.1:2000SEP08 | 407910H1 | 1223 | 1446 |
| 117 | LG:1140501.1:2000SEP08 | 5848628H1 | 1240 | 1432 |
| 117 | LG:1140501.1:2000SEP08 | 3474192H1 | 1245 | 1440 |
| 117 | LG:1140501.1:2000SEP08 | g6133150 | 1269 | 1724 |
| 117 | LG:1140501.1:2000SEP08 | 5101342H1 | 1273 | 1480 |
| 117 | LG:1140501.1:2000SEP08 | 1599286H1 | 1283 | 1477 |
| 117 | LG:1140501.1:2000SEP08 | g562654 | 1288 | 1478 |
| 117 | LG:1140501.1:2000SEP08 | g6400605 | 1294 | 1724 |
| 117 | LG:1140501.1:2000SEP08 | g6400665 | 1304 | 1724 |
| 117 | LG:1140501.1:2000SEP08 | g6663625 | 1306 | 1722 |
| 117 | LG:1140501.1:2000SEP08 | 2152552H1 | 1304 | 1550 |
| 117 | LG:1140501.1:2000SEP08 | g1548344 | 1325 | 1712 |
| 117 | LG:1140501.1:2000SEP08 | 1270380H1 | 1327 | 1546 |
| 117 | LG:1140501.1:2000SEP08 | 1662282H1 | 1328 | 1513 |
| 117 | LG:1140501.1:2000SEP08 | g6400622 | 1329 | 1724 |
| 117 | LG:1140501.1:2000SEP08 | 1662431H1 | 1328 | 1518 |
| 117 | LG:1140501.1:2000SEP08 | g6834934 | 1345 | 1725 |
| 117 | LG:1140501.1:2000SEP08 | g1302886 | 1346 | 1537 |
| 117 | LG:1140501.1:2000SEP08 | g6302011 | 1349 | 1730 |
| 117 | LG:1140501.1:2000SEP08 | g6400692 | 1350 | 1724 |
| 117 | LG:1140501.1:2000SEP08 | g4282315 | 1358 | 1721 |
| 117 | LG:1140501.1:2000SEP08 | 5782350H1 | 1358 | 1498 |
| 117 | LG:1140501.1:2000SEP08 | g3042692 | 1363 | 1721 |
| 117 | LG:1140501.1:2000SEP08 | 242926H1 | 1380 | 1592 |
| 117 | LG:1140501.1:2000SEP08 | g2826024 | 1379 | 1556 |
| 117 | LG:1140501.1:2000SEP08 | g1198673 | 1379 | 1501 |
| 117 | LG:1140501.1:2000SEP08 | g6301497 | 1389 | 1729 |
| 117 | LG:1140501.1:2000SEP08 | g900752 | 1389 | 1720 |
| 117 | LG:1140501.1:2000SEP08 | 7617025J1 | 1390 | 1700 |
| 117 | LG:1140501.1:2000SEP08 | 5442937H1 | 1408 | 1663 |
| 117 | LG:1140501.1:2000SEP08 | 7288888H1 | 1411 | 1539 |
| 117 | LG:1140501.1:2000SEP08 | 3714610H1 | 1411 | 1634 |
| 117 | LG:1140501.1:2000SEP08 | 2564520H1 | 1415 | 1673 |
| 117 | LG:1140501.1:2000SEP08 | 1941138H1 | 1602 | 1712 |
| 117 | LG:1140501.1:2000SEP08 | 7335518H1 | 1522 | 1712 |
| 117 | LG:1140501.1:2000SEP08 | g6047770 | 1534 | 1723 |
| 117 | LG:1140501.1:2000SEP08 | 5371341H1 | 1561 | 1712 |
| 117 | LG:1140501.1:2000SEP08 | g6301731 | 1581 | 1730 |
| 117 | LG:1140501.1:2000SEP08 | 2532761H1 | 1429 | 1712 |
| 117 | LG:1140501.1:2000SEP08 | g958393 | 1463 | 1681 |
| 117 | LG:1140501.1:2000SEP08 | g1801301 | 1499 | 1721 |
| 117 | LG:1140501.1:2000SEP08 | 1501643H1 | 949 | 1109 |
| 117 | LG:1140501.1:2000SEP08 | 4352981H1 | 949 | 1163 |
| 117 | LG:1140501.1:2000SEP08 | 6861727H1 | 952 | 1442 |
| 117 | LG:1140501.1:2000SEP08 | 2267642H1 | 957 | 1115 |
| 117 | LG:1140501.1:2000SEP08 | 3332580H1 | 959 | 1212 |
| 117 | LG:1140501.1:2000SEP08 | 751881H1 | 967 | 1145 |
| 117 | LG:1140501.1:2000SEP08 | 1400206H1 | 967 | 1210 |
| 117 | LG:1140501.1:2000SEP08 | g827443 | 971 | 1122 |
| 117 | LG:1140501.1:2000SEP08 | 1720108H1 | 980 | 1186 |
| 117 | LG:1140501.1:2000SEP08 | g4152386 | 993 | 1430 |
| 117 | LG:1140501.1:2000SEP08 | 1269892H1 | 988 | 1187 |
| 117 | LG:1140501.1:2000SEP08 | 4787624H1 | 1007 | 1165 |
| 117 | LG:1140501.1:2000SEP08 | 4490410H1 | 1 | 474 |
| 117 | LG:1140501.1:2000SEP08 | 1287831H1 | 47 | 279 |
| 117 | LG:1140501.1:2000SEP08 | 1287831F1 | 47 | 586 |
| 117 | LG:1140501.1:2000SEP08 | g1324442 | 98 | 361 |
| 117 | LG:1140501.1:2000SEP08 | 9389948 | 141 | 470 |
| 117 | LG:1140501.1:2000SEP08 | 3647648H1 | 237 | 533 |
| 117 | LG:1140501.1:2000SEP08 | 4615179H1 | 300 | 369 |
| 117 | LG:1140501.1:2000SEP08 | 1617734H1 | 1007 | 1243 |
| 117 | LG:1140501.1:2000SEP08 | 2665537H1 | 1007 | 1237 |
| 117 | LG:1140501.1:2000SEP08 | 036959H1 | 1013 | 1237 |
| 117 | LG:1140501.1:2000SEP08 | 7045262H1 | 477 | 911 |
| 117 | LG:1140501.1:2000SEP08 | 1772758H1 | 1029 | 1287 |
| 117 | LG:1140501.1:2000SEP08 | 7043093H1 | 477 | 1018 |
| 117 | LG:1140501.1:2000SEP08 | 5197443H1 | 1034 | 1109 |
| 117 | LG:1140501.1:2000SEP08 | 7137924H1 | 477 | 909 |
| 117 | LG:1140501.1:2000SEP08 | 301794H1 | 877 | 975 |
| 117 | LG:1140501.1:2000SEP08 | 683911H1 | 881 | 1024 |
| 117 | LG:1140501.1:2000SEP08 | 4060555H1 | 881 | 1054 |
| 117 | LG:1140501.1:2000SEP08 | 1362896H1 | 884 | 1127 |
| 117 | LG:1140501.1:2000SEP08 | 4362919H1 | 894 | 1155 |
| 117 | LG:1140501.1:2000SEP08 | 1788994H1 | 1037 | 1292 |
| 117 | LG:1140501.1:2000SEP08 | 1601350H1 | 918 | 1111 |
| 117 | LG:1140501.1:2000SEP08 | 1596563H1 | 918 | 1038 |
| 117 | LG:1140501.1:2000SEP08 | 1253123H1 | 928 | 1169 |
| 118 | LG:001239.1:2000SEP08 | 5063854H1 | 1901 | 2154 |
| 118 | LG:001239.1:2000SEP08 | 7325215H1 | 1349 | 1845 |
| 118 | LG:001239.1:2000SEP08 | 1616912T6 | 1805 | 2104 |
| 118 | LG:001239.1:2000SEP08 | 4991695H1 | 1380 | 1645 |
| 118 | LG:001239.1:2000SEP08 | 4991695F6 | 1452 | 1645 |
| 118 | LG:001239.1:2000SEP08 | 2722485H1 | 1552 | 1791 |
| 118 | LG:001239.1:2000SEP08 | 5556871H1 | 1699 | 1943 |
| 118 | LG:001239.1:2000SEP08 | 5063854F6 | 1804 | 2154 |
| 118 | LG:001239.1:2000SEP08 | 657641R6 | 1071 | 1432 |
| 118 | LG:001239.1:2000SEP08 | g5554311 | 922 | 1240 |
| 118 | LG:001239.1:2000SEP08 | 652995H1 | 964 | 1178 |
| 118 | LG:001239.1:2000SEP08 | 654098H1 | 1030 | 1117 |
| 118 | LG:001239.1:2000SEP08 | 657641H1 | 1070 | 1279 |
| 118 | LG:001239.1:2000SEP08 | 6778152J1 | 728 | 1332 |
| 118 | LG:001239.1:2000SEP08 | 2432153H1 | 884 | 1117 |
| 118 | LG:001239.1:2000SEP08 | 1709963F6 | 729 | 1136 |
| 118 | LG:001239.1:2000SEP08 | 1709963H1 | 729 | 960 |
| 118 | LG:001239.1:2000SEP08 | 4845124H1 | 1 | 197 |
| 118 | LG:001239.1:2000SEP08 | 7701502H1 | 497 | 1028 |
| 118 | LG:001239.1:2000SEP08 | 7168805H1 | 540 | 1115 |
| 118 | LG:001239.1:2000SEP08 | 7661635H1 | 548 | 1053 |
| 118 | LG:001239.1:2000SEP08 | 4991695T6 | 3 | 629 |
| 118 | LG:001239.1:2000SEP08 | 1912873H1 | 461 | 693 |
| 118 | LG:001239.1:2000SEP08 | 526604H1 | 470 | 740 |
| 118 | LG:001239.1:2000SEP08 | 5063854T6 | 1 | 338 |
| 118 | LG:001239.1:2000SEP08 | 1582560H1 | 554 | 755 |
| 118 | LG:001239.1:2000SEP08 | 658207H1 | 614 | 892 |
| 118 | LG:001239.1:2000SEP08 | 3421048H1 | 547 | 795 |
| 118 | LG:001239.1:2000SEP08 | 656841H1 | 552 | 821 |
| 118 | LG:001239.1:2000SEP08 | 1580231H1 | 554 | 759 |
| 118 | LG:001239.1:2000SEP08 | 527595H1 | 541 | 804 |
| 118 | LG:001239.1:2000SEP08 | 526612H1 | 541 | 786 |
| 119 | LG:018980.1:2000SEP08 | g2839721 | 731 | 1191 |
| 119 | LG:018980.1:2000SEP08 | 7178708H1 | 1 | 438 |
| 119 | LG:018980.1:2000SEP08 | g4895968 | 723 | 1182 |
| 119 | LG:018980.1:2000SEP08 | 6894386H1 | 409 | 969 |
| 119 | LG:018980.1:2000SEP08 | 3158420H1 | 704 | 987 |
| 119 | LG:018980.1:2000SEP08 | 2611701H1 | 951 | 1198 |
| 119 | LG:018980.1:2000SEP08 | 2611701F6 | 951 | 1373 |
| 119 | LG:018980.1:2000SEP08 | g1989719 | 955 | 1284 |
| 119 | LG:018980.1:2000SEP08 | 6855796F8 | 33 | 687 |
| 119 | LG:018980.1:2000SEP08 | 6855796H1 | 33 | 444 |
| 119 | LG:018980.1:2000SEP08 | 5733374H1 | 804 | 1048 |
| 119 | LG:018980.1:2000SEP08 | g3838536 | 351 | 831 |
| 119 | LG:018980.1:2000SEP08 | 5369710F6 | 134 | 583 |
| 119 | LG:018980.1:2000SEP08 | 5369710H1 | 134 | 381 |
| 120 | LG:1083120.3:2000SEP08 | g6132189 | 133 | 385 |
| 120 | LG:1083120.3:2000SEP08 | 5401502T6 | 1 | 364 |
| 120 | LG:1083120.3:2000SEP08 | g6027870 | 50 | 351 |
| 121 | LG:233258.3:2000SEP08 | 7020442H1 | 4154 | 4718 |
| 121 | LG:233258.3:2000SEP08 | g656021 | 4167 | 4329 |
| 121 | LG:233258.3:2000SEP08 | g5445060 | 4187 | 4329 |
| 121 | LG:233258.3:2000SEP08 | g3960731 | 4188 | 4332 |
| 121 | LG:233258.3:2000SEP08 | g656316 | 4261 | 4328 |
| 121 | LG:233258.3:2000SEP08 | 2879658H1 | 4569 | 4839 |
| 121 | LG:233258.3:2000SEP08 | g4685176 | 4074 | 4329 |
| 121 | LG:233258.3:2000SEP08 | g5849471 | 4075 | 4329 |
| 121 | LG:233258.3:2000SEP08 | g5233698 | 4081 | 4342 |
| 121 | LG:233258.3:2000SEP08 | g6834587 | 4081 | 4329 |
| 121 | LG:233258.3:2000SEP08 | g3232700 | 4081 | 4524 |
| 121 | LG:233258.3:2000SEP08 | 5475930F8 | 4105 | 4330 |
| 121 | LG:233258.3:2000SEP08 | 2255632R6 | 4139 | 4335 |
| 121 | LG:233258.3:2000SEP08 | 2255632T6 | 4139 | 4296 |
| 121 | LG:233258.3:2000SEP08 | 1854836T6 | 4144 | 4287 |
| 121 | LG:233258.3:2000SEP08 | g655629 | 4147 | 4330 |
| 121 | LG:233258.3:2000SEP08 | g1507644 | 3991 | 4236 |
| 121 | LG:233258.3:2000SEP08 | g4393553 | 4001 | 4329 |
| 121 | LG:233258.3:2000SEP08 | 1638434F6 | 4004 | 4329 |
| 121 | LG:233258.3:2000SEP08 | g1544747 | 4025 | 4329 |
| 121 | LG:233258.3:2000SEP08 | g3922526 | 4052 | 4330 |
| 121 | LG:233258.3:2000SEP08 | g2238843 | 4052 | 4337 |
| 121 | LG:233258.3:2000SEP08 | g567704 | 4059 | 4329 |
| 121 | LG:233258.3:2000SEP08 | g4739064 | 4074 | 4329 |
| 121 | LG:233258.3:2000SEP08 | g4078093 | 4073 | 4329 |
| 121 | LG:233258.3:2000SEP08 | g6476775 | 4074 | 4329 |
| 121 | LG:233258.3:2000SEP08 | g4690871 | 4074 | 4329 |
| 121 | LG:233258.3:2000SEP08 | 1651244T6 | 4011 | 4287 |
| 121 | LG:233258.3:2000SEP08 | 1638434H1 | 4004 | 4235 |
| 121 | LG:233258.3:2000SEP08 | 1638434T6 | 4008 | 4288 |
| 121 | LG:233258.3:2000SEP08 | 6181441H1 | 4009 | 4278 |
| 121 | LG:233258.3:2000SEP08 | 4898762F6 | 1782 | 2079 |
| 121 | LG:233258.3:2000SEP08 | 4898762H1 | 1782 | 2057 |
| 121 | LG:233258.3:2000SEP08 | 3388815H1 | 1825 | 2097 |
| 121 | LG:233258.3:2000SEP08 | 7260851H1 | 1876 | 2409 |
| 121 | LG:233258.3:2000SEP08 | 6539421H1 | 1878 | 2117 |
| 121 | LG:233258.3:2000SEP08 | 1651244F6 | 3093 | 3587 |
| 121 | LG:233258.3:2000SEP08 | 1651244H1 | 3093 | 3343 |
| 121 | LG:233258.3:2000SEP08 | 4204272H1 | 3111 | 3386 |
| 121 | LG:233258.3:2000SEP08 | 5109856H1 | 3114 | 3377 |
| 121 | LG:233258.3:2000SEP08 | 1639273H1 | 3143 | 3249 |
| 121 | LG:233258.3:2000SEP08 | 6206230H1 | 3143 | 3765 |
| 121 | LG:233258.3:2000SEP08 | g656275 | 3160 | 3441 |
| 121 | LG:233258.3:2000SEP08 | 4574534H1 | 3771 | 4054 |
| 121 | LG:233258.3:2000SEP08 | 1987314H1 | 3773 | 3885 |
| 121 | LG:233258.3:2000SEP08 | g2242553 | 3789 | 4222 |
| 121 | LG:233258.3:2000SEP08 | 2349726T6 | 3794 | 4290 |
| 121 | LG:233258.3:2000SEP08 | 646129H1 | 3809 | 4044 |
| 121 | LG:233258.3:2000SEP08 | 070681H1 | 3811 | 3944 |
| 121 | LG:233258.3:2000SEP08 | 1987314R6 | 3771 | 4069 |
| 121 | LG:233258.3:2000SEP08 | 5475584H1 | 2892 | 3151 |
| 121 | LG:233258.3:2000SEP08 | 3604927H1 | 2956 | 3258 |
| 121 | LG:233258.3:2000SEP08 | 4911767H1 | 2960 | 3241 |
| 121 | LG:233258.3:2000SEP08 | g575173 | 2994 | 3294 |
| 121 | LG:233258.3:2000SEP08 | 3770483H1 | 2999 | 3223 |
| 121 | LG:233258.3:2000SEP08 | 685289H1 | 3024 | 3258 |
| 121 | LG:233258.3:2000SEP08 | 7260921H1 | 3027 | 3511 |
| 121 | LG:233258.3:2000SEP08 | 6777080H1 | 3081 | 3691 |
| 121 | LG:233258.3:2000SEP08 | 4426155H1 | 3086 | 3352 |
| 121 | LG:233258.3:2000SEP08 | 3386329H1 | 3087 | 3328 |
| 121 | LG:233258.3:2000SEP08 | 1456075R1 | 3829 | 4335 |
| 121 | LG:233258.3:2000SEP08 | 1427649T6 | 3846 | 4289 |
| 121 | LG:233258.3:2000SEP08 | 1987314T6 | 3818 | 4290 |
| 121 | LG:233258.3:2000SEP08 | 1482987T6 | 3855 | 4306 |
| 121 | LG:233258.3:2000SEP08 | 683211T6 | 3856 | 4282 |
| 121 | LG:233258.3:2000SEP08 | 2197276T6 | 3881 | 4287 |
| 121 | LG:233258.3:2000SEP08 | 1645077T6 | 3827 | 4289 |
| 121 | LG:233258.3:2000SEP08 | 2266182H1 | 3879 | 4117 |
| 121 | LG:233258.3:2000SEP08 | 5323426F8 | 3815 | 4345 |
| 121 | LG:233258.3:2000SEP08 | g2616449 | 3961 | 4321 |
| 121 | LG:233258.3:2000SEP08 | g2167464 | 3969 | 4329 |
| 121 | LG:233258.3:2000SEP08 | 917564H1 | 3971 | 4142 |
| 121 | LG:233258.3:2000SEP08 | g4217577 | 3985 | 4329 |
| 121 | LG:233258.3:2000SEP08 | g3239990 | 3988 | 4340 |
| 121 | LG:233258.3:2000SEP08 | g6047394 | 3917 | 4329 |
| 121 | LG:233258.3:2000SEP08 | g2669871 | 3922 | 4333 |
| 121 | LG:233258.3:2000SEP08 | g3422044 | 3923 | 4332 |
| 121 | LG:233258.3:2000SEP08 | 6168225H1 | 3934 | 4326 |
| 121 | LG:233258.3:2000SEP08 | 2199941T6 | 3934 | 4296 |
| 121 | LG:233258.3:2000SEP08 | g4897115 | 3935 | 4333 |
| 121 | LG:233258.3:2000SEP08 | 6840521H1 | 3890 | 4335 |
| 121 | LG:233258.3:2000SEP08 | 1642516T6 | 3913 | 4287 |
| 121 | LG:233258.3:2000SEP08 | 4533427T1 | 3895 | 4291 |
| 121 | LG:233258.3:2000SEP08 | g2835576 | 3906 | 4329 |
| 121 | LG:233258.3:2000SEP08 | g5879910 | 3889 | 4329 |
| 121 | LG:233258.3:2000SEP08 | 7728747J1 | 1892 | 2497 |
| 121 | LG:233258.3:2000SEP08 | 7628991H1 | 1905 | 2431 |
| 121 | LG:233258.3:2000SEP08 | 7717561H1 | 1990 | 2623 |
| 121 | LG:233258.3:2000SEP08 | 4900264F6 | 2105 | 2383 |
| 121 | LG:233258.3:2000SEP08 | 4900264H2 | 2105 | 2391 |
| 121 | LG:233258.3:2000SEP08 | 7410352H1 | 2137 | 2678 |
| 121 | LG:233258.3:2000SEP08 | 5426338H1 | 2626 | 2805 |
| 121 | LG:233258.3:2000SEP08 | 4901947H1 | 2753 | 3021 |
| 121 | LG:233258.3:2000SEP08 | 2509912H1 | 2761 | 3000 |
| 121 | LG:233258.3:2000SEP08 | 5475476H1 | 2892 | 3131 |
| 121 | LG:233258.3:2000SEP08 | 7587947H1 | 3589 | 4171 |
| 121 | LG:233258.3:2000SEP08 | 1642516H1 | 3601 | 3776 |
| 121 | LG:233258.3:2000SEP08 | 2922448H1 | 3608 | 3881 |
| 121 | LG:233258.3:2000SEP08 | 241 1669H1 | 3557 | 3800 |
| 121 | LG:233258.3:2000SEP08 | 5119288H1 | 3509 | 3799 |
| 121 | LG:233258.3:2000SEP08 | 5349663H1 | 3513 | 3754 |
| 121 | LG:233258.3:2000SEP08 | 1349048F1 | 3578 | 4053 |
| 121 | LG:233258.3:2000SEP08 | 7687714J1 | 3522 | 3616 |
| 121 | LG:233258.3:2000SEP08 | 3345379H1 | 3531 | 3799 |
| 121 | LG:233258.3:2000SEP08 | 1456075H1 | 3542 | 3780 |
| 121 | LG:233258.3:2000SEP08 | 1349048H1 | 3579 | 3836 |
| 121 | LG:233258.3:2000SEP08 | 4385471H1 | 3582 | 3820 |
| 121 | LG:233258.3:2000SEP08 | 2312950H1 | 3429 | 3675 |
| 121 | LG:233258.3:2000SEP08 | 7057669H1 | 3448 | 4059 |
| 121 | LG:233258.3:2000SEP08 | 2363124H1 | 3448 | 3694 |
| 121 | LG:233258.3:2000SEP08 | 5216535H1 | 3479 | 3765 |
| 121 | LG:233258.3:2000SEP08 | 5640562H1 | 3481 | 3729 |
| 121 | LG:233258.3:2000SEP08 | 4309879H1 | 3478 | 3788 |
| 121 | LG:233258.3:2000SEP08 | 3500829H1 | 3490 | 3771 |
| 121 | LG:233258.3:2000SEP08 | 7580996H1 | 3184 | 3575 |
| 121 | LG:233258.3:2000SEP08 | g317699 | 3198 | 3516 |
| 121 | LG:233258.3:2000SEP08 | 6208746H1 | 3216 | 3477 |
| 121 | LG:233258.3:2000SEP08 | 4165236H1 | 3246 | 3550 |
| 121 | LG:233258.3:2000SEP08 | 4139559H1 | 3307 | 3595 |
| 121 | LG:233258.3:2000SEP08 | 4143858H1 | 3307 | 3578 |
| 121 | LG:233258.3:2000SEP08 | 1482987F6 | 3311 | 3803 |
| 121 | LG:233258.3:2000SEP08 | 1482987H1 | 3311 | 3391 |
| 121 | LG:233258.3:2000SEP08 | 699001H1 | 3373 | 3579 |
| 121 | LG:233258.3:2000SEP08 | 3882343H1 | 3327 | 3546 |
| 121 | LG:233258.3:2000SEP08 | 2349726F6 | 3334 | 3952 |
| 121 | LG:233258.3:2000SEP08 | 2349726H1 | 3334 | 3539 |
| 121 | LG:233258.3:2000SEP08 | 6777575H1 | 3358 | 3936 |
| 121 | LG:233258.3:2000SEP08 | 3498890H1 | 3359 | 3643 |
| 121 | LG:233258.3:2000SEP08 | 7244035H1 | 3385 | 3935 |
| 121 | LG:233258.3:2000SEP08 | g894952 | 3640 | 3790 |
| 121 | LG:233258.3:2000SEP08 | 1691794H1 | 3701 | 3836 |
| 121 | LG:233258.3:2000SEP08 | 5133771H1 | 3674 | 3938 |
| 121 | LG:233258.3:2000SEP08 | g779021 | 3678 | 3931 |
| 121 | LG:233258.3:2000SEP08 | g850896 | 3678 | 3934 |
| 121 | LG:233258.3:2000SEP08 | 4824305H1 | 3696 | 3827 |
| 121 | LG:233258.3:2000SEP08 | 3771947H1 | 3610 | 3885 |
| 121 | LG:233258.3:2000SEP08 | 6311763H1 | 3629 | 4146 |
| 121 | LG:233258.3:2000SEP08 | 5475930T8 | 3629 | 4228 |
| 121 | LG:233258.3:2000SEP08 | 631 1663H1 | 3630 | 4189 |
| 121 | LG:233258.3:2000SEP08 | 4620140H1 | 3629 | 3876 |
| 121 | LG:233258.3:2000SEP08 | 5913558H1 | 3636 | 3912 |
| 121 | LG:233258.3:2000SEP08 | g2167549 | 3637 | 4101 |
| 121 | LG:233258.3:2000SEP08 | 977882H1 | 3758 | 4064 |
| 121 | LG:233258.3:2000SEP08 | 5323426T9 | 3765 | 4223 |
| 121 | LG:233258.3:2000SEP08 | 5021669T1 | 3768 | 4277 |
| 121 | LG:233258.3:2000SEP08 | 4627772H1 | 3710 | 3967 |
| 121 | LG:233258.3:2000SEP08 | 2343388H1 | 3711 | 3968 |
| 121 | LG:233258.3:2000SEP08 | 7628991J1 | 3717 | 4252 |
| 121 | LG:233258.3:2000SEP08 | 2355986H1 | 3738 | 3956 |
| 121 | LG:233258.3:2000SEP08 | 4533427H1 | 3740 | 3897 |
| 121 | LG:233258.3:2000SEP08 | 4706876H1 | 3750 | 4021 |
| 121 | LG:233258.3:2000SEP08 | 977882R1 | 3758 | 4319 |
| 121 | LG:233258.3:2000SEP08 | 5598851H1 | 307 | 562 |
| 121 | LG:233258.3:2000SEP08 | 7324630H1 | 408 | 686 |
| 121 | LG:233258.3:2000SEP08 | 7660821H1 | 524 | 1076 |
| 121 | LG:233258.3:2000SEP08 | 2197276F6 | 584 | 953 |
| 121 | LG:233258.3:2000SEP08 | 1897612H1 | 1 | 326 |
| 121 | LG:233258.3:2000SEP08 | 1897612F6 | 1 | 501 |
| 121 | LG:233258.3:2000SEP08 | 6776039F8 | 78 | 688 |
| 121 | LG:233258.3:2000SEP08 | 7433752H1 | 161 | 667 |
| 121 | LG:233258.3:2000SEP08 | 4458276H1 | 229 | 490 |
| 121 | LG:233258.3:2000SEP08 | 7734751J1 | 271 | 903 |
| 121 | LG:233258.3:2000SEP08 | 6779277H1 | 303 | 715 |
| 121 | LG:233258.3:2000SEP08 | 6776039R8 | 861 | 1469 |
| 121 | LG:233258.3:2000SEP08 | 6776039J1 | 878 | 1494 |
| 121 | LG:233258.3:2000SEP08 | 7176796H1 | 1004 | 1462 |
| 121 | LG:233258.3:2000SEP08 | 7660821J1 | 1176 | 1705 |
| 121 | LG:233258.3:2000SEP08 | 6779277J1 | 1222 | 1791 |
| 121 | LG:233258.3:2000SEP08 | 2880118H1 | 1265 | 1493 |
| 121 | LG:233258.3:2000SEP08 | 7717561J1 | 1365 | 1989 |
| 121 | LG:233258.3:2000SEP08 | 1427649H1 | 1546 | 1795 |
| 121 | LG:233258.3:2000SEP08 | 1427649F6 | 1567 | 1990 |
| 121 | LG:233258.3:2000SEP08 | 1721856H1 | 1592 | 1797 |
| 121 | LG:233258.3:2000SEP08 | 7359946H1 | 1643 | 2184 |
| 121 | LG:233258.3:2000SEP08 | 6259602H1 | 1604 | 1679 |
| 121 | LG:233258.3:2000SEP08 | 2987344H1 | 1681 | 1937 |
| 121 | LG:233258.3:2000SEP08 | 7329967H1 | 826 | 1418 |
| 121 | LG:233258.3:2000SEP08 | 5616252H1 | 688 | 973 |
| 121 | LG:233258.3:2000SEP08 | 2199941F6 | 584 | 790 |
| 121 | LG:233258.3:2000SEP08 | 2199941H1 | 584 | 834 |
| 121 | LG:233258.3:2000SEP08 | 2197276H1 | 584 | 818 |
| 121 | LG:233258.3:2000SEP08 | 7261410H1 | 618 | 1237 |
| 121 | LG:233258.3:2000SEP08 | g3163278 | 640 | 1003 |
| 121 | LG:233258.3:2000SEP08 | 7595923H1 | 2139 | 2739 |
| 121 | LG:233258.3:2000SEP08 | 6456564H1 | 2153 | 2732 |
| 121 | LG:233258.3:2000SEP08 | 6351657H1 | 2218 | 2541 |
| 121 | LG:233258.3:2000SEP08 | 6777080J1 | 2298 | 2876 |
| 121 | LG:233258.3:2000SEP08 | 6777575J1 | 2478 | 3097 |
| 121 | LG:233258.3:2000SEP08 | 6809274H1 | 2315 | 2761 |
| 121 | LG:233258.3:2000SEP08 | 6809274H1 | 2315 | 2761 |
| 121 | LG:233258.3:2000SEP08 | g564218 | 2493 | 2651 |
| 121 | LG:233258.3:2000SEP08 | 683211H1 | 2399 | 2666 |
| 121 | LG:233258.3:2000SEP08 | 7745596H1 | 2515 | 3057 |
| 121 | LG:233258.3:2000SEP08 | 7371787H1 | 2520 | 3034 |
| 121 | LG:233258.3:2000SEP08 | 687106H1 | 2399 | 2653 |
| 121 | LG:233258.3:2000SEP08 | 7260937H1 | 2581 | 3160 |
| 121 | LG:233258.3:2000SEP08 | 683211R6 | 2399 | 2638 |
| 121 | LG:233258.3:2000SEP08 | 7728747H1 | 2416 | 3060 |
| 121 | LG:233258.3:2000SEP08 | 3467934H1 | 2453 | 2702 |
| 121 | LG:233258.3:2000SEP08 | 1645077H1 | 2455 | 2669 |
| 121 | LG:233258.3:2000SEP08 | 1645077F6 | 2455 | 2883 |
| 121 | LG:233258.3:2000SEP08 | 3986427H1 | 2463 | 2739 |
| 121 | LG:233258.3:2000SEP08 | 6777134J1 | 2478 | 3125 |
| 121 | LG:233258.3:2000SEP08 | 1854836H1 | 2592 | 2839 |
| 121 | LG:233258.3:2000SEP08 | 1854036H1 | 2592 | 2876 |
| 121 | LG:233258.3:2000SEP08 | 1854836F6 | 2592 | 2936 |
| 122 | LG:999062.1:2000SEP08 | 6268754H1 | 1 | 563 |
| 122 | LG:999062.1:2000SEP08 | 626875418 | 1 | 594 |
| 122 | LG:999062.1:2000SEP08 | 6268754F8 | 18 | 682 |
| 123 | LG:887776.1:2000SEP08 | 5985557H1 | 5 | 96 |
| 123 | LG:887776.1:2000SEP08 | 7381148H1 | 261 | 852 |
| 123 | LG:887776.1:2000SEP08 | 6045524H1 | 1776 | 1997 |
| 123 | LG:887776.1:2000SEP08 | 6080103H1 | 1586 | 1923 |
| 123 | LG:887776.1:2000SEP08 | 5338564T8 | 1626 | 1783 |
| 123 | LG:887776.1:2000SEP08 | 7381784H1 | 1 | 540 |
| 123 | LG:887776.1:2000SEP08 | 5338555F8 | 1238 | 1817 |
| 123 | LG:887776.1:2000SEP08 | 5338555H1 | 1238 | 1375 |
| 123 | LG:887776.1:2000SEP08 | 5338564H1 | 1239 | 1450 |
| 123 | LG:887776.1:2000SEP08 | 5335668T8 | 1562 | 1832 |
| 123 | LG:887776.1:2000SEP08 | 6454788H1 | 1485 | 1991 |
| 123 | LG:887776.1:2000SEP08 | 5338555T8 | 1507 | 1886 |
| 123 | LG:887776.1:2000SEP08 | 6601228F8 | 1556 | 2010 |
| 123 | LG:887776.1:2000SEP08 | 6601228H1 | 1556 | 1999 |
| 123 | LG:887776.1:2000SEP08 | 660122818 | 1556 | 1908 |
| 123 | LG:887776.1:2000SEP08 | 5335668H1 | 455 | 630 |
| 123 | LG:887776.1:2000SEP08 | 5335668F8 | 455 | 1051 |
| 123 | LG:887776.1:2000SEP08 | 7380908H1 | 525 | 1117 |
| 123 | LG:887776.1:2000SEP08 | 6453407H1 | 863 | 1426 |
| 123 | LG:887776.1:2000SEP08 | 5952782H1 | 886 | 1181 |
| 123 | LG:887776.1:2000SEP08 | 5338564F8 | 1239 | 1812 |
| 124 | LG:1400301.2:2000SEP08 3796761F6 | 1 | 403 | |
| 124 | LG:1400301.2:2000SEP08 3796761H1 | 1 | 306 | |
| 124 | LG:1400301.2:2000SEP08 4654776T6 | 126 | 637 | |
| 124 | LG:1400301.2:2000SEP08 | g895564 | 59 | 388 |
| 124 | LG:1400301.2:2000SEP08 4654776F6 | 34 | 415 | |
| 124 | LG:1400301.2:2000SEP08 1729735H1 | 52 | 280 | |
| 124 | LG:1400301.2:2000SEP08 4654776H1 | 34 | 285 | |
| 125 | LG:1329362.1:2000SEP08 | g1734408 | 55 | 460 |
| 125 | LG:1329362.1:2000SEP08 7697049J1 | 156 | 405 | |
| 125 | LG:1329362.1:2000SEP08 6490895R9 | 1 | 461 | |
| 125 | LG:1329362.1:2000SEP08 5564064H1 | 240 | 445 | |
| 125 | LG:1329362.1:2000SEP08 7697049H1 | 167 | 405 | |
| 126 | LG:1096498.1:2000SEP08 6798228H1 | 1 | 331 | |
| 126 | LG:1096498.1:2000SEP08 6792384H1 | 1 | 502 | |
| 126 | LG:1096498.1:2000SEP08 6792384F8 | 1 | 579 | |
| 126 | LG:1096498.1:2000SEP08 6790975T8 | 8 | 281 | |
| 126 | LG:1096498.1:2000SEP08 6790975H1 | 8 | 403 | |
| 126 | LG: 1096498.1 :2000SEP08 | 6790975F8 | 8 | 402 |
| 127 | LG:1096337.1:2000SEP08 | g812407 | 542 | 897 |
| 127 | LG:1096337.1:2000SEP08 | 3668515T6 | 251 | 782 |
| 127 | LG:1096337.1:2000SEP08 | 9812493 | 379 | 638 |
| 127 | LG:1096337.1:2000SEP08 | 6451060H1 | 51 | 513 |
| 127 | LG:1096337.1:2000SEP08 | 5665201H1 | 398 | 508 |
| 127 | LG:1096337.1:2000SEP08 | 3668515F6 | 1 | 492 |
| 128 | LG:1400579.1:2000SEP08 | 6895912H1 | 1 | 353 |
| 128 | LG:1400579.1:2000SEP08 | 6895912F8 | 1 | 353 |
| 128 | LG:1400579.1:2000SEP08 | 7388585H1 | 89 | 627 |
| 128 | LG:1400579.1:2000SEP08 | 1251881H1 | 111 | 367 |
| 128 | LG:1400579.1:2000SEP08 | 6895946F8 | 185 | 353 |
| 128 | LG: 1400579.1:2000SEP08 | 5547349H1 | 358 | 577 |
| 128 | LG:1400579.1:2000SEP08 | 697696T6 | 583 | 914 |
| 128 | LG:1400579.1:2000SEP08 | 697696R6 | 595 | 914 |
| 128 | LG:1400579.1:2000SEP08 | 697696H1 | 598 | 773 |
| 128 | LG:1400579.1:2000SEP08 | 696573H1 | 595 | 813 |
| 128 | LG:1400579.1:2000SEP08 | 4418332H1 | 149 | 394 |
| 128 | LG:1400579.1:2000SEP08 | 6895946H1 | 1 | 353 |
| 128 | LG:1400579.1:2000SEP08 | 477786F1 | 251 | 865 |
| 128 | LG:1400579.1:2000SEP08 | 3449269H1 | 361 | 504 |
| 128 | LG:1400579.1:2000SEP08 | 3898628H1 | 1 | 241 |
| 128 | LG:1400579.1:2000SEP08 | 477786T6 | 433 | 830 |
| 129 | LG:1080091.1:2000SEP08 | 5484791H2 | 7 | 250 |
| 129 | LG:1080091.1:2000SEP08 | 4850844H1 | 7 | 286 |
| 129 | LG:1080091.1:2000SEP08 | 1274729H1 | 7 | 268 |
| 129 | LG:1080091.1:2000SEP08 | 5491715H1 | 7 | 257 |
| 129 | LG:1080091.1:2000SEP08 | 3069676H1 | 30 | 286 |
| 129 | LG:1080091.1:2000SEP08 | 212172H1 | 274 | 455 |
| 129 | LG:1080091.1:2000SEP08 | 7345695H1 | 1 | 551 |
| 129 | LG:1080091.1:2000SEP08 | 4850844F6 | 7 | 477 |
| 129 | LG:1080091.1:2000SEP08 | 1274729F6 | 7 | 285 |
| 129 | LG:1080091.1:2000SEP08 | 3596975H1 | 17 | 300 |
| 129 | LG:1080091.1:2000SEP08 | 5189963H1 | 31 | 299 |
| 129 | LG:1080091.1:2000SEP08 | 2887410H1 | 28 | 283 |
| 129 | LG:1080091.1:2000SEP08 | 2662961H1 | 16 | 252 |
| 129 | LG:1080091.1:2000SEP08 | 3069676F6 | 30 | 229 |
| 129 | LG:1080091.1:2000SEP08 | 5078633H1 | 26 | 274 |
| 129 | LG:1080091.1:2000SEP08 | g3434079 | 181 | 285 |
| 130 | LG:1082203.1:2000SEP08 | 3770530F6 | 907 | 1374 |
| 130 | LG:1082203.1:2000SEP08 | 6255271H1 | 730 | 986 |
| 130 | LG:1082203.1:2000SEP08 | 2877410H1 | 1038 | 1321 |
| 130 | LG:1082203.1:2000SEP08 | 3497137T6 | 934 | 1471 |
| 130 | LG:1082203.1:2000SEP08 | 7430791H1 | 977 | 1540 |
| 130 | LG:1082203.1:2000SEP08 | 4216384T6 | 997 | 1673 |
| 130 | LG:1082203.1:2000SEP08 | g5436703 | 1058 | 1510 |
| 130 | LG:1082203.1:2000SEP08 | 2881835T6 | 1097 | 1658 |
| 130 | LG:1082203.1:2000SEP08 | 5802512H1 | 1118 | 1404 |
| 130 | LG:1082203.1:2000SEP08 | 5542972H1 | 1210 | 1412 |
| 130 | LG:1082203.1:2000SEP08 | 3216760T6 | 1388 | 1662 |
| 130 | LG:1082203.1:2000SEP08 | 7766080J1 | 569 | 1181 |
| 130 | LG:1082203.1:2000SEP08 | 5529680H1 | 961 | 1238 |
| 130 | LG:1082203.1:2000SEP08 | 3497137F6 | 524 | 1061 |
| 130 | LG:1082203.1:2000SEP08 | 065669H1 | 699 | 986 |
| 130 | LG:1082203.1:2000SEP08 | 6489704R9 | 1560 | |
| 130 | LG:1082203.1:2000SEP08 | 4023626F8 | 69 | 658 |
| 130 | LG:1082203.1:2000SEP08 | 6615979H1 | 497 | 921 |
| 130 | LG:1082203.1:2000SEP08 | 1233083H1 | 539 | 745 |
| 130 | LG:1082203.1:2000SEP08 | 4708380H1 | 783 | 1045 |
| 130 | LG:1082203.1:2000SEP08 | g1099997 | 791 | 1046 |
| 130 | LG:1082203.1:2000SEP08 | 3216760H1 | 832 | 1108 |
| 130 | LG:1082203.1:2000SEP08 | 3216760F6 | 833 | 1324 |
| 130 | LG:1082203.1:2000SEP08 | 2881835H1 | 459 | 722 |
| 130 | LG:1082203.1:2000SEP08 | 3497137H1 | 525 | 810 |
| 130 | LG:1082203.1:2000SEP08 | 2881835F6 | 457 | 823 |
| 130 | LG:1082203.1:2000SEP08 | 4707980H1 | 783 | 988 |
| 130 | LG:1082203.1:2000SEP08 | 3026964H1 | 1012 | 1128 |
| 131 | LG:1084051.1:2000SEP08 | 5217490H1 | 1 | 198 |
| 131 | LG:1084051.1:2000SEP08 | 1542833H1 | 1 | 137 |
| 131 | LG:1084051.1:2000SEP08 | 3836068F6 | 7 | 549 |
| 131 | LG:1084051.1:2000SEP08 | 3052342H1 | 46 | 335 |
| 131 | LG:1084051.1:2000SEP08 | g5661058 | 155 | 347 |
| 131 | LG:1084051.1:2000SEP08 | 3525414H1 | 171 | 494 |
| 131 | LG:1084051.1:2000SEP08 | 7113909H1 | 182 | 747 |
| 131 | LG:1084051.1:2000SEP08 | 6182691T8 | 227 | 873 |
| 131 | LG:1084051.1:2000SEP08 | 3679568T9 | 546 | 925 |
| 131 | LG:1084051.1:2000SEP08 | 3836068T6 | 560 | 1044 |
| 131 | LG:1084051.1:2000SEP08 | 4315370T9 | 585 | 850 |
| 131 | LG:1084051.1:2000SEP08 | 3022533T6 | 568 | 902 |
| 131 | LG:1084051.1:2000SEP08 | 3021710R6 | 568 | 943 |
| 131 | LG:1084051.1:2000SEP08 | 6371853H1 | 587 | 865 |
| 131 | LG:1084051.1:2000SEP08 | g4737276 | 630 | 1082 |
| 131 | LG:1084051.1:2000SEP08 | 7108014H1 | 774 | 1258 |
| 131 | LG:1084051.1:2000SEP08 | 6018614H1 | 102 | 700 |
| 131 | LG:1084051.1:2000SEP08 | 6182691F8 | 334 | 815 |
| 131 | LG:1084051.1:2000SEP08 | 5735738H1 | 78 | 348 |
| 131 | LG:1084051.1:2000SEP08 | 1891884H1 | 19 | 264 |
| 131 | LG:1084051.1:2000SEP08 | 1895443H1 | 19 | 272 |
| 131 | LG:1084051.1:2000SEP08 | 4729485H1 | 86 | 235 |
| 131 | LG:1084051.1:2000SEP08 | 3021710H1 | 568 | 853 |
| 131 | LG:1084051.1:2000SEP08 | 3021038T6 | 568 | 880 |
| 131 | LG:1084051.1:2000SEP08 | 3836068H1 | 7 | 289 |
| 132 | LG:1082393.1:2000SEP08 | 4129648H1 | 1189 | 1391 |
| 132 | LG:1082393.1:2000SEP08 | 6118209F8 | 1050 | 1590 |
| 132 | LG:1082393.1:2000SEP08 | 4129281F6 | 1189 | 1637 |
| 132 | LG:1082393.1:2000SEP08 | 2041133H1 | 1341 | 1576 |
| 132 | LG:1082393.1:2000SEP08 | 7235041H1 | 1357 | 1907 |
| 132 | LG:1082393.1:2000SEP08 | 7626695J1 | 1370 | 1963 |
| 132 | LG:1082393.1:2000SEP08 | 3392427T8 | 1407 | 1953 |
| 132 | LG:1082393.1:2000SEP08 | g1291764 | 1425 | 1959 |
| 132 | LG:1082393.1:2000SEP08 | 4181819T8 | 1554 | 2024 |
| 132 | LG:1082393.1:2000SEP08 | 6051495R8 | 1570 | 2175 |
| 132 | LG:1082393.1:2000SEP08 | 6118412T8 | 1709 | 2206 |
| 132 | LG:1082393.1:2000SEP08 | 6118209H1 | 1048 | 1543 |
| 132 | LG:1082393.1:2000SEP08 | 1691842H1 | 781 | 1020 |
| 132 | LG:1082393.1:2000SEP08 | 5004434H1 | 1076 | 1286 |
| 132 | LG:1082393.1:2000SEP08 | 7275193H1 | 1 | 456 |
| 132 | LG:1082393.1:2000SEP08 | 7740514H1 | 13 | 431 |
| 132 | LG:1082393.1:2000SEP08 | 5814635T8 | 35 | 590 |
| 132 | LG:1082393.1:2000SEP08 | 4820753F6 | 38 | 605 |
| 132 | LG:1082393.1:2000SEP08 | 7438990H1 | 46 | 456 |
| 132 | LG:1082393.1:2000SEP08 | 6919464F6 | 44 | 616 |
| 132 | LG:1082393.1:2000SEP08 | 7160272H1 | 47 | 452 |
| 132 | LG:1082393.1:2000SEP08 | g4895686 | 291 | 692 |
| 132 | LG:1082393.1:2000SEP08 | g5366722 | 365 | 733 |
| 132 | LG:1082393.1:2000SEP08 | 774051411 | 409 | 1070 |
| 132 | LG:1082393.1:2000SEP08 | 4200727F8 | 518 | 1091 |
| 132 | LG:1082393.1:2000SEP08 | 424948H1 | 558 | 783 |
| 132 | LG:1082393.1:2000SEP08 | 3392427H1 | 597 | 885 |
| 132 | LG:1082393.1:2000SEP08 | 6244520F8 | 597 | 1264 |
| 132 | LG:1082393.1:2000SEP08 | 7626695H1 | 658 | 1192 |
| 132 | LG:1082393.1:2000SEP08 | 6121404H1 | 1048 | 1519 |
| 132 | LG:1082393.1:2000SEP08 | 6051387J1 | 1662 | 2175 |
| 132 | LG:1082393.1:2000SEP08 | 5813479F8 | 8 | 619 |
| 132 | LG:1082393.1:2000SEP08 | 3500086H1 | 1252 | 1450 |
| 132 | LG:1082393.1:2000SEP08 | g6567918 | 453 | 686 |
| 132 | LG:1082393.1:2000SEP08 | g6700463 | 1626 | 1968 |
| 132 | LG:1082393.1:2000SEP08 | g6567913 | 267 | 687 |
| 132 | LG:1082393.1:2000SEP08 | 5814635F8 | 8 | 617 |
| 132 | LG:1082393.1:2000SEP08 | 1691166H1 | 964 | 1056 |
| 132 | LG:1082393.1:2000SEP08 | 1694272F7 | 781 | 1380 |
| 132 | LG:1082393.1:2000SEP08 | 6560051F8 | 697 | 1208 |
| 132 | LG:1082393.1:2000SEP08 | 6121404F8 | 1048 | 1620 |
| 132 | LG:1082393.1:2000SEP08 | 5813882F8 | 8 | 541 |
| 132 | LG:1082393.1:2000SEP08 | 7618303H1 | 855 | 1451 |
| 132 | LG:1082393.1:2000SEP08 | 6051387H1 | 1037 | 1621 |
| 132 | LG:1082393.1:2000SEP08 | 1543372H1 | 1134 | 1351 |
| 132 | LG:1082393.1:2000SEP08 | 2056575H1 | 1193 | 1441 |
| 132 | LG:1082393.1:2000SEP08 | 7219966H1 | 22 | 564 |
| 132 | LG:1082393.1:2000SEP08 | g1921318 | 1207 | 1506 |
| 132 | LG:1082393.1:2000SEP08 | 6051495F8 | 1037 | 1451 |
| 132 | LG:1082393.1:2000SEP08 | 6118412H1 | 1048 | 1543 |
| 132 | LG:1082393.1:2000SEP08 | 7090489H1 | 55 | 519 |
| 132 | LG:1082393.1:2000SEP08 | 5819550F8 | 8 | 482 |
| 132 | LG:1082393.1:2000SEP08 | 4129281H2 | 1189 | 1440 |
| 132 | LG:1082393.1:2000SEP08 | 5919204H1 | 1226 | 1516 |
| 132 | LG:1082393.1:2000SEP08 | 6560051H1 | 697 | 1236 |
| 132 | LG:1082393.1:2000SEP08 | 5813882T8 | 104 | 588 |
| 132 | LG:1082393.1:2000SEP08 | 6919464H1 | 49 | 608 |
| 132 | LG:1082393.1:2000SEP08 | g2112377 | 697 | 1126 |
| 132 | LG:1082393.1:2000SEP08 | g1745471 | 1361 | 1749 |
| 132 | LG:1082393.1:2000SEP08 | 7249510H1 | 1318 | 1842 |
| 132 | LG:1082393.1:2000SEP08 | 6720311H1 | 464 | 1101 |
| 132 | LG:1082393.1:2000SEP08 | 5819550T8 | 181 | 590 |
| 132 | LG:1082393.1:2000SEP08 | 6118412F8 | 1048 | 1572 |
| 132 | LG:1082393.1:2000SEP08 | 3392402H1 | 600 | 885 |
| 132 | LG:1082393.1:2000SEP08 | 6051495H1 | 1037 | 1512 |
| 133 | LG:1086183.1:2000SEP08 | 5086621H1 | 42 | 308 |
| 133 | LG:1086183.1:2000SEP08 | 3561959H1 | 113 | 411 |
| 133 | LG:1086183.1:2000SEP08 | 4738571F6 | 1 | 399 |
| 133 | LG:1086183.1:2000SEP08 | 5078969H1 | 76 | 323 |
| 133 | LG:1086183.1:2000SEP08 | 3161081H1 | 103 | 368 |
| 133 | LG:1086183.1:2000SEP08 | 2070126T6 | 512 | 951 |
| 133 | LG:1086183.1:2000SEP08 | 20701 26F6 | 76 | 431 |
| 133 | LG:1086183.1:2000SEP08 | 6985304H1 | 219 | 386 |
| 133 | LG:1086183.1:2000SEP08 | 20701 26H1 | 76 | 302 |
| 133 | LG:1086183.1:2000SEP08 | 5078969F8 | 76 | 670 |
| 133 | LG:1086183.1:2000SEP08 | 6985304R8 | 218 | 763 |
| 133 | LG:1086183.1:2000SEP08 | 6985304F8 | 218 | 766 |
| 133 | LG:1086183.1:2000SEP08 | g316570 | 1 | 206 |
| 133 | LG:1086183.1:2000SEP08 | 4738571H1 | 1 | 263 |
| 133 | LG:1086183.1:2000SEP08 | 4739279F6 | 36 | 474 |
| 133 | LG:1086183.1:2000SEP08 | 036692R7 | 63 | 432 |
| 133 | LG:1086183.1:2000SEP08 | 5883746F7 | 49 | 668 |
| 133 | LG:1086183.1:2000SEP08 | 036692H1 | 64 | 283 |
| 133 | LG:1086183.1:2000SEP08 | 2070125F6 | 76 | 442 |
| 133 | LG:1086183.1:2000SEP08 | 2579218H1 | 498 | 730 |
| 133 | LG:1086183.1:2000SEP08 | 2653825H1 | 687 | 972 |
| 133 | LG:1086183.1:2000SEP08 | 3215936H1 | 695 | 959 |
| 133 | LG:1086183.1:20003EP08 | 6124139F8 | 735 | 1319 |
| 133 | LG:1086183.1:2000SEP08 | 588374617 | 753 | 874 |
| 133 | LG:1086183.1:2000SEP08 | 7194315H1 | 289 | 560 |
| 133 | LG:1086183.1:2000SEP08 | 2070125T6 | 470 | 882 |
| 133 | LG:1086183.1:2000SEP08 5621149R6 | 440 | 704 | |
| 133 | LG:1086183.1:2000SEP08 2070125H1 | 76 | 303 | |
| 133 | LG:1086183.1:2000SEP08 4739279H1 | 36 | 302 | |
| 133 | LG:1086183.1:2000SEP08 5883746H1 | 50 | 185 | |
| 133 | LG:1086183.1:2000SEP08 5883225H1 | 50 | 365 | |
| 133 | LG:1086183.1:2000SEP08 6124139H1 | 735 | 1267 | |
| 134 | LG:1090268.1:2000SEP08 | g761185 | 1611 | 1889 |
| 134 | LG:1090268.1:2000SEP08 | g6451646 | 1257 | 1704 |
| 134 | LG:1090268.1:2000SEP08 | 6754141J1 | 674 | 1187 |
| 134 | LG:1090268.1:2000SEP08 | g6474819 | 710 | 1147 |
| 134 | LG:1090268.1:2000SEP08 | g6026190 | 710 | 1196 |
| 134 | LG:1090268.1:2000SEP08 | g1548583 | 864 | 1188 |
| 134 | LG:1090268.1:2000SEP08 | g3075725 | 1208 | 1587 |
| 134 | LG:1090268.1:2000SEP08 | g3415945 | 1193 | 1533 |
| 134 | LG:1090268.1:2000SEP08 | g1023497 | 1848 | 2159 |
| 134 | LG:1090268.1:2000SEP08 | g3751784 | 753 | 1196 |
| 134 | LG:1090268.1:2000SEP08 | g5877749 | 753 | 1175 |
| 134 | LG:1090268.1:2000SEP08 | g3116962 | 860 | 1196 |
| 134 | LG:1090268.1:2000SEP08 | g2183774 | 1195 | 1616 |
| 134 | LG:1090268.1:2000SEP08 | 7604119J1 | 1208 | 1768 |
| 134 | LG:1090268.1:2000SEP08 | g2881166 | 753 | 1045 |
| 134 | LG:1090268.1:2000SEP08 | g4086766 | 1189 | 1423 |
| 134 | LG:1090268.1:2000SEP08 | g2183859 | 2094 | 2483 |
| 134 | LG:1090268.1:2000SEP08 | g1516679 | 2064 | 2483 |
| 134 | LG:1090268.1:2000SEP08 | g2900164 | 774 | 1126 |
| 134 | LG:1090268.1:2000SEP08 | g5630905 | 768 | 1178 |
| 134 | LG:1090268.1:2000SEP08 | g5707045 | 767 | 1178 |
| 134 | LG:1090268.1:2000SEP08 | g3244577 | 1208 | 1556 |
| 134 | LG:1090268.1:2000SEP08 | g3835326 | 765 | 1196 |
| 134 | LG:1090268.1:2000SEP08 | g5543649 | 1241 | 1573 |
| 134 | LG:1090268.1:2000SEP08 | g4222719 | 765 | 1196 |
| 134 | LG:1090268.1:2000SEP08 | g6837062 | 1239 | 1576 |
| 134 | LG:1090268.1:2000SEP08 | g5746714 | 762 | 1187 |
| 134 | LG:1090268.1:2000SEP08 | g3737064 | 802 | 911 |
| 134 | LG:1090268.1:2000SEP08 | g4373166 | 762 | 1176 |
| 134 | LG:1090268.1:2000SEP08 | g4524999 | 835 | 1196 |
| 134 | LG:1090268.1:2000SEP08 | g5510939 | 806 | 1175 |
| 134 | LG:1090268.1:2000SEP08 | g3674851 | 806 | 1157 |
| 134 | LG:1090268.1:2000SEP08 | g4175704 | 806 | 1196 |
| 134 | LG:1090268.1:2000SEP08 | g4124656 | 806 | 1178 |
| 134 | LG:1090268.1:2000SEP08 | g5448195 | 800 | 1196 |
| 134 | LG:1090268.1:2000SEP08 | g2753708 | 789 | 1129 |
| 134 | LG:1090268.1:2000SEP08 | g2817239 | 1257 | 1502 |
| 134 | LG:1090268.1:2000SEP08 | g5395904 | 1211 | 1669 |
| 134 | LG:1090268.1:2000SEP08 | g4622023 | 869 | 1196 |
| 134 | LG:1090268.1:2000SEP08 | g3752837 | 722 | 1196 |
| 134 | LG:1090268.1:2000SEP08 | g4223947 | 1189 | 1679 |
| 134 | LG:1090268.1:2000SEP08 | g1479211 | 902 | 1193 |
| 134 | LG:1090268.1:2000SEP08 | g2141283 | 1189 | 1473 |
| 134 | LG:1090268.1:2000SEP08 | g2840299 | 1190 | 1627 |
| 134 | LG:1090268.1:2000SEP08 | 7601734J1 | 981 | 1180 |
| 134 | LG:1090268.1:2000SEP08 | 893366H1 | 1846 | 2102 |
| 134 | LG:1090268.1:2000SEP08 | g1445330 | 853 | 1188 |
| 134 | LG:1090268.1:2000SEP08 | g6399975 | 2211 | 2351 |
| 134 | LG:1090268.1:2000SEP08 | g2183965 | 2159 | 2483 |
| 134 | LG:1090268.1:2000SEP08 | g846167 | 2311 | 2483 |
| 134 | LG:1090268.1:2000SEP08 | g855476 | 1251 | 1586 |
| 134 | LG:1090268.1:2000SEP08 | 3244065H1 | 2290 | 2482 |
| 134 | LG:1090268.1:2000SEP08 | 2233146H1 | 2139 | 2360 |
| 134 | LG:1090268.1:2000SEP08 | 2568814H1 | 1826 | 2076 |
| 134 | LG:1090268.1:2000SEP08 | 2152933H1 | 1606 | 1858 |
| 134 | LG:1090268.1:2000SEP08 | 3144079H1 | 1277 | 1586 |
| 134 | LG:1090268.1:2000SEP08 | 1732920H1 | 1930 | 2144 |
| 134 | LG:1090268.1:2000SEP08 | 7718061J1 | 650 | 1265 |
| 134 | LG:1090268.1:2000SEP08 | g4373019 | 742 | 1196 |
| 134 | LG:1090268.1:2000SEP08 | g3431675 | 1207 | 1652 |
| 134 | LG:1090268.1:2000SEP08 | g4522588 | 741 | 1196 |
| 134 | LG:1090268.1:2000SEP08 | g3003118 | 1189 | 1336 |
| 134 | LG: 1090268.1:2000SEP08 | g3934836 | 738 | 1178 |
| 134 | LG:1090268.1:2000SEP08 | g1483699 | 1189 | 1317 |
| 134 | LG:1090268.1:2000SEP08 | g5396099 | 1208 | 1664 |
| 134 | LG:1090268.1:2000SEP08 | g4852321 | 902 | 1196 |
| 134 | LG:1090268.1:2000SEP08 | 2903867F6 | 877 | 1051 |
| 134 | LG:1090268.1:2000SEP08 | 6754670J1 | 639 | 1189 |
| 134 | LG:1090268.1:2000SEP08 | g2841837 | 1218 | 1587 |
| 134 | LG:1090268.1:2000SEP08 | g3797598 | 754 | 1196 |
| 134 | LG:1090268.1:2000SEP08 | 1574256H1 | 2148 | 2377 |
| 134 | LG:1090268.1:2000SEP08 | 2492240H1 | 2209 | 2482 |
| 134 | LG:1090268.1:2000SEP08 | 1306727F6 | 1355 | 1876 |
| 134 | LG:1090268.1:2000SEP08 | 1617117H1 | 833 | 1035 |
| 134 | LG:1090268.1:2000SEP08 | 2095111H1 | 2030 | 2319 |
| 134 | LG:1090268.1:2000SEP08 | 1760228H1 | 1769 | 2031 |
| 134 | LG:1090268.1:2000SEP08 | g2563663 | 1255 | 1463 |
| 134 | LG:1090268.1:2000SEP08 | 1304083H1 | 2028 | 2245 |
| 134 | LG:1090268.1:2000SEP08 | 1909588H1 | 2297 | 2482 |
| 134 | LG:1090268.1:2000SEP08 | 1213940H1 | 2097 | 2316 |
| 134 | LG:1090268.1:2000SEP08 | g786692 | 1236 | 1488 |
| 134 | LG:1090268.1:2000SEP08 | g2818092 | 982 | 1126 |
| 134 | LG:1090268.1:2000SEP08 | g4282074 | 1094 | 1187 |
| 134 | LG:1090268.1:2000SEP08 | g1898035 | 909 | 1196 |
| 134 | LG:1090268.1:2000SEP08 | g2818846 | 985 | 1130 |
| 134 | LG:1090268.1:2000SEP08 | g2817406 | 912 | 1101 |
| 134 | LG:1090268.1:2000SEP08 | g2910074 | 903 | 1110 |
| 134 | LG:1090268.1:2000SEP08 | g2903216 | 913 | 1132 |
| 134 | LG:1090268.1:2000SEP08 | g3835163 | 900 | 1196 |
| 134 | LG:1090268.1:2000SEP08 | g2877817 | 897 | 1129 |
| 134 | LG:1090268.1:2000SEP08 | g3181114 | 984 | 1196 |
| 134 | LG:1090268.1:2000SEP08 | g5545123 | 700 | 1196 |
| 134 | LG:1090268.1:2000SEP08 | g5592081 | 930 | 1178 |
| 134 | LG:1090268.1:2000SEP08 | g6438602 | 731 | 1196 |
| 134 | LG:1090268.1:2000SEP08 6753494H1 | 319 | 804 | |
| 134 | LG:1090268.1:2000SEP08 6753494J1 | 581 | 1187 | |
| 134 | LG:1090268.1:2000SEP08 2930018H1 | 640 | 942 | |
| 134 | LG:1090268.1:2000SEP08 | 4960753H1 | 674 | 947 |
| 134 | LG:1090268.1:2000SEP08 | g4971679 | 698 | 1196 |
| 134 | LG:1090268.1:2000SEP08 | 7706430J1 | 726 | 1307 |
| 134 | LG:1090268.1:2000SEP08 | 2903867H1 | 877 | 1165 |
| 134 | LG:1090268.1:2000SEP08 | 7763068J1 | 980 | 1195 |
| 134 | LG:1090268.1:2000SEP08 | g2754250 | 1195 | 1642 |
| 134 | LG:1090268.1:2000SEP08 | 6770449J1 | 1198 | 1745 |
| 134 | LG:1090268.1:2000SEP08 | 4111913H1 | 1237 | 1534 |
| 134 | LG:1090268.1:2000SEP08 | 3180441H1 | 1271 | 1571 |
| 134 | LG:1090268.1:2000SEP08 | 4277537H1 | 1271 | 1545 |
| 134 | LG:1090268.1:2000SEP08 | 3144349H1 | 1277 | 1595 |
| 134 | LG:1090268.1:2000SEP08 | 6824119J1 | 1315 | 1982 |
| 134 | LG:1090268.1:2000SEP08 | 7763068H1 | 1350 | 1827 |
| 134 | LG:1090268.1:2000SEP08 | 7348839H1 | 1405 | 1982 |
| 134 | LG:1090268.1:20003EP08 | 2541932H1 | 1572 | 1806 |
| 134 | LG:1090268.1:2000SEP08 | g2184071 | 1601 | 2026 |
| 134 | LG:1090268.1:2000SEP08 | 063284H1 | 1669 | 1844 |
| 134 | LG:1090268.1:2000SEP08 | g786737 | 1714 | 1983 |
| 134 | LG:1090268.1:2000SEP08 | 6812555R8 | 1790 | 1934 |
| 134 | LG:1090268.1:2000SEP08 | 503446H1 | 1831 | 2058 |
| 134 | LG:1090268.1:2000SEP08 | 2109222H1 | 1832 | 2073 |
| 134 | LG:1090268.1:2000SEP08 | 3480694H1 | 1849 | 1968 |
| 134 | LG:1090268.1:2000SEP08 | 7308968H1 | 1935 | 2484 |
| 134 | LG:1090268.1:2000SEP08 | 345564H1 | 1966 | 2178 |
| 134 | LG:1090268.1:2000SEP08 | 3495146H1 | 311 | 534 |
| 134 | LG:1090268.1:2000SEP08 | g810697 | 2015 | 2250 |
| 134 | LG:1090268.1:2000SEP08 | 1323858H1 | 2041 | 2292 |
| 134 | LG:1090268.1:2000SEP08 | 1323858T6 | 2041 | 2445 |
| 134 | LG:1090268.1:2000SEP08 | g2069240 | 2061 | 2485 |
| 134 | LG:1090268.1:2000SEP08 | 504232H1 | 2143 | 2359 |
| 134 | LG:1090268.1:2000SEP08 | 2753977H1 | 2210 | 2483 |
| 134 | LG:1090268.1:2000SEP08 | 3181838H1 | 2221 | 2484 |
| 134 | LG:1090268.1:2000SEP08 | g3422817 | 1193 | 1566 |
| 134 | LG:1090268.1:20003EP08 | g1892524 | 20 | 475 |
| 134 | LG:1090268.1:2000SEP08 | 3020237H1 | 170 | 452 |
| 134 | LG:1090268.1:2000SEP08 | 7461969H1 | 187 | 717 |
| 134 | LG:1090268.1:2000SEP08 | 3514175H1 | 195 | 452 |
| 134 | LG:1090268.1:2000SEP08 | 7718061H1 | 1 | 563 |
| 134 | LG:1090268.1:2000SEP08 | 7706430H1 | 237 | 792 |
| 134 | LG:1090268.1:2000SEP08 | 2304939H1 | 1670 | 1895 |
| 134 | LG:1090268.1:2000SEP08 | g5513894 | 719 | 1196 |
| 134 | LG:1090268.1:2000SEP08 | g678609 | 2077 | 2478 |
| 134 | LG:1090268.1:2000SEP08 | g1483703 | 1189 | 1579 |
| 134 | LG:1090268.1:2000SEP08 | g3595778 | 1207 | 1667 |
| 134 | LG:1090268.1:2000SEP08 | g2848875 | 1189 | 1499 |
| 134 | LG:1090268.1:2000SEP08 | g855586 | 1619 | 1983 |
| 134 | LG:1090268.1:2000SEP08 | g5395903 | 1205 | 1663 |
| 134 | LG:1090268.1:2000SEP08 7606141J1 | 1200 | 1786 | |
| 134 | LG:1090268.1:2000SEP08 | g1516773 | 1670 | 2113 |
| 134 | LG:1090268.1:2000SEP08 | g5395662 | 1207 | 1679 |
| 134 | LG:1090268.1:2000SEP08 | g5367381 | 721 | 1175 |
| 134 | LG:1090268.1:2000SEP08 | g3645380 | 1189 | 1674 |
| 134 | LG:1090268.1:2000SEP08 | 839547H1 | 2258 | 2482 |
| 134 | LG:1090268.1:2000SEP08 | g5363202 | 1207 | 1667 |
| 134 | LG:1090268.1:2000SEP08 | g2458340 | 1189 | 1615 |
| 134 | LG:1090268.1:2000SEP08 | g5904809 | 1220 | 1666 |
| 134 | LG:1090268.1:2000SEP08 | g6451781 | 1189 | 1489 |
| 134 | LG:1090268.1:2000SEP08 | g5887491 | 1189 | 1509 |
| 134 | LG:1090268.1:2000SEP08 | 063491H1 | 1669 | 1883 |
| 134 | LG:1090268.1:2000SEP08 | g1064322 | 914 | 1150 |
| 134 | LG:1090268.1:2000SEP08 | g3147590 | 884 | 1196 |
| 134 | LG:1090268.1:2000SEP08 | g6704625 | 893 | 1195 |
| 134 | LG:1090268.1:2000SEP08 | g2554235 | 850 | 1115 |
| 134 | LG:1090268.1:2000SEP08 | g1925854 | 1067 | 1173 |
| 134 | LG:1090268.1:2000SEP08 | g4486399 | 849 | 1196 |
| 134 | LG:1090268.1:2000SEP08 | g2783700 | 846 | 1144 |
| 134 | LG:1090268.1:2000SEP08 | g5513830 | 840 | 1196 |
| 134 | LG:1090268.1:2000SEP08 | g5812533 | 840 | 1196 |
| 134 | LG:1090268.1:2000SEP08 | g3279089 | 838 | 1196 |
| 134 | LG:1090268.1:2000SEP08 | g6462439 | 837 | 1196 |
| 134 | LG:1090268.1:2000SEP08 | g6043117 | 837 | 1187 |
| 134 | LG:1090268.1:2000SEP08 | g2993463 | 1241 | 1412 |
| 134 | LG:1090268.1:2000SEP08 | g5526060 | 808 | 1150 |
| 134 | LG:1090268.1:2000SEP08 | g810364 | 1639 | 1912 |
| 134 | LG:1090268.1:2000SEP08 | g726667 | 1959 | 2182 |
| 134 | LG:1090268.1:2000SEP08 | g846116 | 1216 | 1508 |
| 134 | LG:1090268.1:2000SEP08 | 842557H1 | 2325 | 2482 |
| 134 | LG:1090268.1:2000SEP08 | g3086781 | 1189 | 1525 |
| 134 | LG:1090268.1:2000SEP08 | 1306727H1 | 1355 | 1566 |
| 134 | LG:1090268.1:2000SEP08 | g761083 | 2232 | 2470 |
| 134 | LG:1090268.1:2000SEP08 | g810267 | 2228 | 2482 |
| 134 | LG:1090268.1:2000SEP08 | g4899638 | 721 | 1196 |
| 134 | LG:1090268.1:2000SEP08 | g3430748 | 720 | 1178 |
| 134 | LG:1090268.1:2000SEP08 | g2817276 | 696 | 1088 |
| 134 | LG:1090268.1:2000SEP08 3289524H1 | 1326 | 1569 | |
| 134 | LG:1090268.1:2000SEP08 | 063093H1 | 1669 | 1909 |
| 134 | LG:1090268.1:2000SEP08 | 1684795H1 | 2294 | 2482 |
| 134 | LG:1090268.1:2000SEP08 | g4535972 | 756 | 1196 |
| 134 | LG:1090268.1:2000SEP08 | g810598 | 2221 | 2483 |
| 134 | LG:1090268.1:2000SEP08 | g5110273 | 720 | 1196 |
| 134 | LG:1090268.1:2000SEP08 | g2881573 | 1260 | 1617 |
| 134 | LG:1090268.1:2000SEP08 | g4851194 | 748 | 1196 |
| 134 | LG:1090268.1:2000SEP08 | g1970707 | 1193 | 1467 |
| 134 | LG:1090268.1:2000SEP08 | g2884408 | 1215 | 1617 |
| 134 | LG:1090268.1:2000SEP08 | 1704927H1 | 1982 | 2183 |
| 134 | LG:1090268.1:2000SEP08 | 389097H1 | 1892 | 2040 |
| 134 | LG:1090268.1:2000SEP08 | g1477294 | 1189 | 1391 |
| 134 | LG:1090268.1:2000SEP08 | g5446508 | 700 | 1196 |
| 134 | LG:1090268.1:2000SEP08 | g1023498 | 2096 | 2472 |
| 134 | LG:1090268.1:2000SEP08 | g3086610 | 1189 | 1713 |
| 134 | LG:1090268.1:2000SEP08 6753471R8 | 591 | 1187 | |
| 134 | LG:1090268.1:2000SEP08 | 737337H1 | 1574 | 1806 |
| 134 | LG:1090268.1:2000SEP08 6308005H1 | 1875 | 1943 | |
| 134 | LG:1090268.1:2000SEP08 | g2732602 | 1188 | 1472 |
| 134 | LG:1090268.1:2000SEP08 | g1023507 | 1848 | 2152 |
| 134 | LG:1090268.1:2000SEP08 | g1634136 | 1590 | 1983 |
| 134 | LG:1090268.1:2000SEP08 | g5675999 | 493 | 707 |
| 134 | LG:1090268.1:2000SEP08 | g901373 | 1746 | 2119 |
| 134 | LG:1090268.1:2000SEP08 | g1114673 | 2128 | 2461 |
| 134 | LG:1090268.1:2000SEP08 | g680642 | 1802 | 2070 |
| 134 | LG:1090268.1:2000SEP08 6753471J1 | 584 | 1189 | |
| 134 | LG:1090268.1:2000SEP08 | 691673H1 | 1662 | 1869 |
| 134 | LG:1090268.1:2000SEP08 6754141R8 | 753 | 1186 | |
| 134 | LG:1090268.1:2000SEP08 | g6197565 | 1189 | 1684 |
| 135 | LG:1400597.5:2000SEP08 | g6025620 | 1 | 396 |
| 135 | LG:1400597.5:2000SEP08 | 250371 3T6 | 1 | 308 |
| 136 | LG:1080307.2:2000SEP08 7734018H2 | 1 | 575 | |
| 137 | LG:1400603.2:2000SEP08 6949927H1 | 18 | 605 | |
| 137 | LG:1400603.2:2000SEP08 7129806R8 | 748 | 1195 | |
| 137 | LG:1400603.2:2000SEP08 | 1257339H1 | 172 | 409 |
| 137 | LG:1400603.2:2000SEP08 | g5451096 | 176 | 626 |
| 137 | LG:1400603.2:2000SEP08 | 1398471F6 | 177 | 586 |
| 137 | LG:1400603.2:2000SEP08 | g3245052 | 228 | 519 |
| 137 | LG:1400603.2:2000SEP08 2694772H1 | 302 | 513 | |
| 137 | LG:1400603.2:2000SEP08 6449019H1 | 330 | 932 | |
| 137 | LG:1400603.2:2000SEP08 | g6837153 | 346 | 765 |
| 137 | LG:1400603.2:2000SEP08 | 7740723H1 | 691 | 1296 |
| 137 | LG:1400603.2:2000SEP08 | 6949927R8 | 706 | 1300 |
| 137 | LG:1400603.2:2000SEP08 | 6768303J1 | 747 | 1319 |
| 137 | LG:1400603.2:2000SEP08 | 4178665F6 | 785 | 1307 |
| 137 | LG:1400603.2:2000SEP08 4178665H1 | 786 | 1047 | |
| 137 | LG:1400603.2:2000SEP08 | 1290821H1 | 975 | 1223 |
| 137 | LG:1400603.2:2000SEP08 | 7265187H1 | 1 | 570 |
| 137 | LG:1400603.2:2000SEP08 | 6949927F8 | 18 | 653 |
| 137 | LG:1400603.2:2000SEP08 | g4690049 | 24 | 371 |
| 137 | LG:1400603.2:2000SEP08 | 1592931T6 | 53 | 588 |
| 137 | LG:1400603.2:2000SEP08 3035748H1 | 152 | 433 | |
| 137 | LG:1400603.2:2000SEP08 | 7740723J1 | 167 | 744 |
| 137 | LG:1400603.2:2000SEP08 6304330H2 | 21 | 520 | |
| 137 | LG:1400603.2:2000SEP08 | 7265118H1 | 12 | 534 |
| 137 | LG:1400603.2:2000SEP08 | 6768403H1 | 16 | 482 |
| 137 | LG:1400603.2:2000SEP08 | 6609870H2 | 40 | 608 |
| 137 | LG:1400603.2:2000SEP08 | 7129806H1 | 748 | 1318 |
| 138 | LG:1052984.1:2000SEP08 | 651414H1 | 16 | 252 |
| 138 | LG:1052984.1:2000SEP08 | 5486139H1 | 2 | 263 |
| 138 | LG:1052984.1:2000SEP08 | 7315588H1 | 1 | 538 |
| 138 | LG:1052984.1:2000SEP08 | 2939892H1 | 14 | 274 |
| 138 | LG:1052984.1:2000SEP08 | g1924344 | 20 | 197 |
| 138 | LG:1052984.1:2000SEP08 3832110F8 | 28 | 683 | |
| 138 | LG:1052984.1:2000SEP08 3600686F6 | 32 | 623 | |
| 138 | LG:1052984.1:2000SEP08 | g1152158 | 38 | 474 |
| 138 | LG:1052984.1:2000SEP08 | 5743693R9 | 65 | 632 |
| 138 | LG:1052984.1:2000SEP08 | g4072524 | 263 | 626 |
| 138 | LG:1052984.1:2000SEP08 | 2550578H1 | 291 | 531 |
| 138 | LG:1052984.1:2000SEP08 | 3600686T6 | 293 | 920 |
| 138 | LG:1052984.1:2000SEP08 | 2737435T6 | 420 | 617 |
| 138 | LG:1052984.1:2000SEP08 | g2031599 | 67 | 399 |
| 138 | LG:1052984.1:2000SEP08 | 3494034H1 | 14 | 284 |
| 138 | LG:1052984.1:2000SEP08 | 3832110H1 | 25 | 298 |
| 138 | LG:1052984.1:2000SEP08 | 5387444H1 | 20 | 287 |
| 138 | LG:1052984.1:2000SEP08 | 641592H1 | 10 | 263 |
| 138 | LG:1052984.1:2000SEP08 | 2958179H1 | 17 | 261 |
| 138 | LG:1052984.1:2000SEP08 | 6369702H1 | 18 | 498 |
| 138 | LG:1052984.1:2000SEP08 | g1492368 | 28 | 219 |
| 138 | LG:1052984.1:2000SEP08 | 2737435F6 | 19 | 501 |
| 138 | LG:1052984.1:2000SEP08 | 6537993H1 | 31 | 606 |
| 138 | LG:1052984.1:2000SEP08 | 3600686H1 | 32 | 318 |
| 138 | LG:1052984.1:2000SEP08 | 5743693H1 | 65 | 356 |
| 138 | LG:1052984.1:2000SEP08 | 2737435H1 | 19 | 226 |
| 138 | LG:1052984.1:2000SEP08 | 5486139F6 | 2 | 523 |
| 139 | LG:1091259.1:2000SEP08 | 7677744H2 | 454 | 982 |
| 139 | LG:1091259.1:2000SEP08 | 4715503H1 | 1 | 221 |
| 139 | LG:1091259.1:2000SEP08 | 6359173H1 | 4 | 542 |
| 139 | LG:1091259.1:2000SEP08 | 5172487F6 | 463 | 982 |
| 139 | LG:1091259.1:2000SEP08 | 4372229H1 | 661 | 935 |
| 139 | LG:1091259.1:2000SEP08 | 5638745H1 | 880 | 982 |
| 139 | LG:1091259.1:2000SEP08 | 4715503F8 | 1 | 550 |
| 139 | LG:1091259.1:2000SEP08 | 7217670H1 | 51 | 582 |
| 139 | LG:1091259.1:2000SEP08 | 4715503F7 | 1 | 544 |
| 139 | LG:1091259.1:2000SEP08 | 4372229F6 | 661 | 982 |
| 139 | LG:1091259.1:2000SEP08 | 7336856H1 | 192 | 793 |
| 139 | LG:1091259.1:2000SEP08 | 5172487H1 | 463 | 701 |
| 140 | LG:1082263.2:2000SEP08 | g1377161 | 978 | 1350 |
| 140 | LG:1082263.2:2000SEP08 | 6331250H1 | 287 | 898 |
| 140 | LG:1082263.2:2000SEP08 | 6054886H1 | 949 | 1519 |
| 140 | LG:1082263.2:2000SEP08 | g2689864 | 640 | 943 |
| 140 | LG:1082263.2:2000SEP08 | 5089517H1 | 1229 | 1537 |
| 140 | LG:1082263.2:2000SEP08 | 4571225H1 | 1252 | 1532 |
| 140 | LG:1082263.2:2000SEP08 | 4571225F7 | 1253 | 1764 |
| 140 | LG:1082263.2:2000SEP08 | 1849907F6 | 1281 | 1679 |
| 140 | LG:1082263.2:2000SEP08 | 6331250T8 | 1367 | 1780 |
| 140 | LG:1082263.2:2000SEP08 | 491570R7 | 1 | 440 |
| 140 | LG:1082263.2:2000SEP08 | 6198216T8 | 74 | 346 |
| 140 | LG:1082263.2:2000SEP08 | 2878502H1 | 74 | 384 |
| 140 | LG:1082263.2:2000SEP08 | 2878502F6 | 73 | 591 |
| 140 | LG:1082263.2:2000SEP08 | 6197337H1 | 79 | 450 |
| 140 | LG:1082263.2:2000SEP08 | g5368781 | 142 | 535 |
| 140 | LG:1082263.2:2000SEP08 | 4384384H1 | 213 | 347 |
| 140 | LG:1082263.2:2000SEP08 | 6331250F8 | 285 | 913 |
| 140 | LG:1082263.2:2000SEP08 | 7613142J1 | 538 | 1050 |
| 140 | LG:1082263.2:2000SEP08 | 4029293H1 | 548 | 786 |
| 140 | LG:1082263.2:2000SEP08 | 4029293F8 | 548 | 932 |
| 140 | LG:1082263.2:2000SEP08 | 4676879H1 | 560 | 826 |
| 140 | LG:1082263.2:2000SEP08 | 1348730H1 | 807 | 990 |
| 140 | LG:1082263.2:2000SEP08 | 6124822F8 | 922 | 1468 |
| 140 | LG:1082263.2:2000SEP08 | g1887432 | 1112 | 1496 |
| 140 | LG:1082263.2:2000SEP08 | 2666231T6 | 1164 | 1687 |
| 140 | LG:1082263.2:2000SEP08 | 1403760H1 | 1169 | 1366 |
| 140 | LG:1082263.2:2000SEP08 | 7287944H1 | 1170 | 1302 |
| 140 | LG:1082263.2:2000SEP08 | 6124822H1 | 913 | 1357 |
| 140 | LG:1082263.2:2000SEP08 | g2904589 | 142 | 520 |
| 140 | LG:1082263.2:2000SEP08 | g1933867 | 629 | 1032 |
| 140 | LG:1082263.2:2000SEP08 | 257020H1 | 82 | 349 |
| 140 | LG:1082263.2:2000SEP08 | 2990552F6 | 87 | 495 |
| 140 | LG:1082263.2:2000SEP08 | 1403760F6 | 1169 | 1567 |
| 140 | LG:1082263.2:2000SEP08 | 7032020H1 | 997 | 1310 |
| 140 | LG:1082263.2:2000SEP08 | 1916902H1 | 1346 | 1611 |
| 140 | LG:1082263.2:2000SEP08 | 1849907H1 | 1281 | 1442 |
| 140 | LG:1082263.2:2000SEP08 | 2990552H1 | 87 | 283 |
| 140 | LG:1082263.2:2000SEP08 | g3230500 | 143 | 460 |
| 140 | LG:1082263.2:2000SEP08 | 6197337F8 | 91 | 441 |
| 140 | LG:1082263.2:2000SEP08 | g2204741 | 1230 | 1632 |
| 141 | LG:1048604.2:2000SEP08 | g2837787 | 383 | 465 |
| 141 | LG:1048604.2:2000SEP08 | 4252763F8 | 1 | 500 |
| 141 | LG:1048604.2:2000SEP08 | 4252763R8 | 26 | 602 |
| 141 | LG:1048604.2:2000SEP08 | 5767951F8 | 225 | 808 |
| 141 | LG:1048604.2:2000SEP08 | 6840843H1 | 679 | 1285 |
| 141 | LG:1048604.2:2000SEP08 | 6837578H1 | 898 | 1285 |
| 141 | LG:1048604.2:2000SEP08 | 6837261H1 | 820 | 1285 |
| 141 | LG:1048604.2:2000SEP08 | 5767951H1 | 225 | 606 |
| 142 | LG:1085254.3:2000SEP08 | 7214794H1 | 324 | 851 |
| 142 | LG:1085254.3:2000SEP08 | g4069986 | 418 | 854 |
| 142 | LG:1085254.3:2000SEP08 | 5048141R8 | 1 | 323 |
| 142 | LG:1085254.3:2000SEP08 | 179261R1 | 48 | 539 |
| 142 | LG:1085254.3:2000SEP08 | 179261R6 | 47 | 451 |
| 142 | LG:1085254.3:2000SEP08 | 7664077J1 | 322 | 877 |
| 142 | LG:1085254.3:2000SEP08 | 7214657H1 | 322 | 897 |
| 142 | LG:1085254.3:2000SEP08 | g4069988 | 401 | 855 |
| 143 | LG:1400606.2:2000SEP08 | 5798223H1 | 739 | 1070 |
| 143 | LG:1400606.2:2000SEP08 | 1459526H1 | 769 | 998 |
| 143 | LG:1400606.2:2000SEP08 | 3879554H1 | 805 | 1033 |
| 143 | LG:1400606.2:2000SEP08 | g669395 | 898 | 1183 |
| 143 | LG:1400606.2:2000SEP08 | g3988920 | 476 | 932 |
| 143 | LG:1400606.2:2000SEP08 | 2512112H1 | 826 | 1147 |
| 143 | LG:1400606.2:2000SEP08 | g2824819 | 857 | 1167 |
| 143 | LG:1400606.2:2000SEP08 | g884098 | 887 | 1261 |
| 143 | LG:1400606.2:2000SEP08 | 063489H1 | 935 | 1129 |
| 143 | LG:1400606.2:2000SEP08 | 117874R1 | 369 | 829 |
| 143 | LG:1400606.2:2000SEP08 | 6946885H1 | 978 | 1298 |
| 143 | LG:1400606.2:2000SEP08 | 6442528H1 | 1 | 516 |
| 143 | LG:1400606.2:2000SEP08 | 3081595F6 | 169 | 414 |
| 143 | LG:1400606.2:2000SEP08 | 681953AJ1 | 314 | 903 |
| 143 | LG:1400606.2:2000SEP08 | 117874H1 | 366 | 597 |
| 143 | LG:1400606.2:2000SEP08 | 3081595H1 | 197 | 466 |
| 143 | LG:1400606.2:2000SEP08 | 117874R6 | 379 | 825 |
| 143 | LG:1400606.2:2000SEP08 | 1459526F6 | 769 | 1070 |
| 143 | LG:1400606.2:2000SEP08 | g669394 | 898 | 1083 |
| 143 | LG:1400606.2:2000SEP08 | 5798223F7 | 738 | 1070 |
| 143 | LG:1400606.2:2000SEP08 | g673044 | 898 | 1120 |
| 143 | LG:1400606.2:2000SEP08 | 6442528F8 | 13 | 518 |
| 143 | LG:1400606.2:2000SEP08 | 5928832F8 | 248 | 682 |
| 144 | LG:1090358.2:2000SEP08 | g5741053 | 367 | 581 |
| 144 | LG:1090358.2:2000SEP08 | g4536188 | 361 | 585 |
| 144 | LG:1090358.2:2000SEP08 | 6478543H1 | 715 | 1149 |
| 144 | LG:1090358.2:2000SEP08 | 6910793J1 | 313 | 925 |
| 144 | LG:1090358.2:2000SEP08 | 505833R6 | 453 | 710 |
| 144 | LG:1090358.2:2000SEP08 | g6576993 | 263 | 710 |
| 144 | LG:1090358.2:2000SEP08 | g2540081 | 257 | 710 |
| 144 | LG:1090358.2:2000SEP08 | 505833F1 | 453 | 710 |
| 144 | LG:1090358.2:2000SEP08 | 505847R7 | 249 | 700 |
| 144 | LG:1090358.2:2000SEP08 | 1726344T6 | 117 | 671 |
| 144 | LG:1090358.2:2000SEP08 | 505833T6 | 453 | 668 |
| 144 | LG:1090358.2:2000SEP08 | g2583400 | 160 | 656 |
| 144 | LG:1090358.2:2000SEP08 | 3841840T8 | 1 | 639 |
| 144 | LG:1090358.2:2000SEP08 | 50584717 | 249 | 637 |
| 144 | LG:1090358.2:2000SEP08 | 3015762H1 | 275 | 561 |
| 144 | LG:1090358.2:2000SEP08 | 6156187H1 | 130 | 452 |
| 144 | LG:1090358.2:2000SEP08 | g6117148 | 356 | 581 |
| 144 | LG:1090358.2:2000SEP08 | g4983712 | 346 | 710 |
| 144 | LG:1090358.2:2000SEP08 | 505833H1 | 453 | 651 |
| 145 | LG:1079064.2:2000SEP08 | 7191108H2 | 39 | 631 |
| 145 | LG:1079064.2:2000SEP08 | 6999243R8 | 1 | 657 |
| 145 | LG:1079064.2:2000SEP08 | 4696877F8 | 11 | 583 |
| 145 | LG:1079064.2:2000SEP08 | 7578884H1 | 27 | 508 |
| 145 | LG:1079064.2:2000SEP08 | 3038074F6 | 43 658 | |
| 145 | LG:1079064.2:2000SEP08 | 7583916H1 | 42 | 629 |
| 145 | LG:1079064.2:2000SEP08 | 7017183H1 | 44 | 638 |
| 145 | LG:1079064.2:2000SEP08 | 6605987H1 | 73 | 661 |
| 145 | LG:1079064.2:2000SEP08 | g917354 | 226 | 548 |
| 145 | LG:1079064.2:2000SEP08 | 6247848T8 | 394 | 917 |
| 145 | LG:1079064.2:2000SEP08 | 5963556F8 | 484 | 1029 |
| 145 | LG:1079064.2:2000SEP08 | 5994871F8 | 525 | 1127 |
| 145 | LG:1079064.2:2000SEP08 | 5808655F8 | 525 | 1098 |
| 145 | LG:1079064.2:2000SEP08 | 5393866F7 | 527 | 1123 |
| 145 | LG:1079064.2:2000SEP08 | 7654781H1 | 545 | 1044 |
| 145 | LG:1079064.2:2000SEP08 | 5531790H1 | 586 | 838 |
| 145 | LG:1079064.2:2000SEP08 | 7178988H1 | 58 | 588 |
| 145 | LG:1079064.2:2000SEP08 | 6247848F8 | 45 | 720 |
| 145 | LG:1079064.2:2000SEP08 | 7188233H1 | 29 | 559 |
| 145 | LG:1079064.2:20003EP08 | 7654781 J 1 | 286 | 662 |
| 145 | LG:1079064.2:2000SEP08 | 7738722H1 | 43 | 703 |
| 145 | LG:1079064.2:2000SEP08 | 7260311H1 | 421 | 895 |
| 145 | LG:1079064.2:2000SEP08 | 7642987H1 | 128 | 324 |
| 145 | LG:1079064.2:2000SEP08 | 7758240J1 | 107 | 512 |
| 145 | LG:1079064.2:2000SEP08 | g6301088 | 80 | 362 |
| 145 | LG:1079064.2:2000SEP08 | 7176120H1 | 11 | 546 |
| 145 | LG:1079064.2:2000SEP08 | 1389713H1 | 384 | 632 |
| 145 | LG:1079064.2:2000SEP08 | 5963556H1 | 484 | 1008 |
| 145 | LG:1079064.2:2000SEP08 | 7580135H1 | 29 | 483 |
| 145 | LG:1079064.2:2000SEP08 | 7581585H1 | 73 | 619 |
| 145 | LG:1079064.2:2000SEP08 | 7189302H2 | 69 | 644 |
| 145 | LG:1079064.2:2000SEP08 | 6445475H2 | 137 | 658 |
| 145 | LG:1079064.2:2000SEP08 | 6298888F7 | 71 | 698 |
| 145 | LG:1079064.2:2000SEP08 | 5994871H1 | 525 | 810 |
| 145 | LG:1079064.2:2000SEP08 | 6247848H1 | 45 | 586 |
| 145 | LG:1079064.2:2000SEP08 | 6929423H1 | 338 | 798 |
| 145 | LG:1079064.2:2000SEP08 | 7267188H2 | 45 | 531 |
| 145 | LG:1079064.2:2000SEP08 | 6929423F8 | 338 | 933 |
| 145 | LG:1079064.2:2000SEP08 | 7461231H2 | 79 | 496 |
| 145 | LG:1079064.2:2000SEP08 | 7367949H1 | 43 | 616 |
| 146 | LG:1076866.1:2000SEP08 | 5879062H1 | 1062 | 1314 |
| 146 | LG:1076866.1:2000SEP08 | 264599H1 | 1182 | 1430 |
| 146 | LG:1076866.1:2000SEP08 | 4830902H1 | 1600 | 1674 |
| 146 | LG:1076866.1:2000SEP08 | 5879056H1 | 1062 | 1322 |
| 146 | LG:1076866.1:2000SEP08 | 2939523H1 | 1791 | 2057 |
| 146 | LG:1076866.1:2000SEP08 | 184111H1 | 1476 | 1707 |
| 146 | LG:1076866.1:2000SEP08 | 3174078H1 | 97 | 329 |
| 146 | LG:1076866.1:2000SEP08 | 2585657F6 | 206 | 419 |
| 146 | LG:1076866.1:2000SEP08 | 6742253H1 | 1198 | 1653 |
| 146 | LG:1076866.1:2000SEP08 | 6919910H1 | 1657 | 2117 |
| 146 | LG:1076866.1:2000SEP08 | 625782R1 | 1796 | 2292 |
| 146 | LG:1076866.1:2000SEP08 | 1001517R1 | 1984 | 2476 |
| 146 | LG:1076866.1:2000SEP08 | 1001517H1 | 1984 | 2228 |
| 146 | LG:1076866.1:2000SEP08 | 3957124H2 | 2035 | 2322 |
| 146 | LG:1076866.1:2000SEP08 | 7107946H1 | 1 | 488 |
| 146 | LG:1076866.1:2000SEP08 | 3174078F6 | 91 | 623 |
| 146 | LG:1076866.1:2000SEP08 | 4323175H1 | 303 | 560 |
| 146 | LG:1076866.1:2000SEP08 | 4323175F7 | 303 | 899 |
| 146 | LG:1076866.1:2000SEP08 | 432317517 | 332 | 816 |
| 146 | LG:1076866.1:2000SEP08 | g5850461 | 457 | 919 |
| 146 | LG:1076866.1:2000SEP08 | g3837638 | 556 | 944 |
| 146 | LG:1076866.1:2000SEP08 | 3174078T6 | 573 | 881 |
| 146 | LG:1076866.1:2000SEP08 | 3778843F9 | 797 | 1255 |
| 146 | LG:1076866.1:2000SEP08 | 3688101H1 | 1114 | 1344 |
| 146 | LG:1076866.1:2000SEP08 | 3688101F6 | 1114 | 1516 |
| 146 | LG:1076866.1:2000SEP08 | 6765719J1 | 1291 | 1870 |
| 146 | LG:1076866.1:2000SEP08 | 1543396R61477 | 1987 | |
| 146 | LG:1076866.1:2000SEP08 | 6078450H1 | 1574 | 1858 |
| 146 | LG:1076866.1:2000SEP08 | 4830902F8 | 1600 | 2178 |
| 146 | LG:1076866.1:2000SEP08 | 1697599H1 | 1610 | 1747 |
| 146 | LG:1076866.1:2000SEP08 | g5439960 | 519 | 944 |
| 146 | LG:1076866.1:2000SEP08 | 625782R6 | 1796 | 2053 |
| 146 | LG:1076866.1:2000SEP08 | 1543396H1 | 1477 | 1682 |
| 146 | LG:1076866.1:2000SEP08 | 6742253F8 | 1205 | 1744 |
| 146 | LG:1076866.1:2000SEP08 | 4830902F9 | 1600 | 2157 |
| 146 | LG:1076866.1:2000SEP08 | 625782H1 | 1796 | 2039 |
| 146 | LG:1076866.1:2000SEP08 | 4764175H1 | 1755 | 2006 |
| 147 | LG:969359.1:2000SEP08 | 6795715H1 | 3 | 510 |
| 147 | LG:969359.1:2000SEP08 | 6798649F8 | 3 | 488 |
| 147 | LG:969359.1:2000SEP08 | 6798649H1 | 3 | 299 |
| 147 | LG:969359.1:2000SEP08 | 6795715F8 | 3 | 571 |
| 147 | LG:969359.1:2000SEP08 | 6793426H1 | 10 | 454 |
| 147 | LG:969359.1:2000SEP08 | 6790981T8 | 169 | 792 |
| 147 | LG:969359.1:2000SEP08 | 6292801H1 | 303 | 449 |
| 147 | LG:969359.1:2000SEP08 | 6798649T8 | 599 | 773 |
| 147 | LG:969359.1:2000SEP08 | 6793426T8 | 657 | 779 |
| 147 | LG:969359.1:2000SEP08 | g1616171 | 710 | 900 |
| 147 | LG:969359.1:2000SEP08 | 6790981H1 | 1 486 | |
| 147 | LG:969359.1:2000SEP08 | 6790981F8 | 1 | 497 |
| 148 | LG:366783.1:2000SEP08 | g1068298 | 1080 | 1313 |
| 148 | LG:366783.1:2000SEP08 | g1068059 | 1069 | 1310 |
| 148 | LG:366783.1:2000SEP08 | 4948403T6 | 621 | 1232 |
| 148 | LG:366783.1:2000SEP08 | 4948403F6 | 441 | 948 |
| 148 | LG:366783.1:2000SEP08 | 4948403H1 | 441 | 676 |
| 148 | LG:366783.1:2000SEP08 | 4600759H1 | 286 | 543 |
| 148 | LG:366783.1:2000SEP08 | 5763587T7 | 1 472 | |
| 149 | LG:332176.3:2000SEP08 | 6623593H1 | 203 | 786 |
| 149 | LG:332176.3:2000SEP08 | 7338995H1 | 662 | 1225 |
| 149 | LG:332176.3:2000SEP08 | 6623593J1 | 731 | 1329 |
| 149 | LG:332176.3:2000SEP08 | 6326238T7 | 856 | 1019 |
| 149 | LG:332176.3:2000SEP08 | 7618143J1 | 1 | 467 |
| 149 | LG:332176.3:2000SEP08 | g1975879 | 32 | 318 |
| 149 | LG:332176.3:2000SEP08 | 1467511T7 | 100 | 537 |
| 149 | LG:332176.3:2000SEP08 | 6326238H1 | 107 | 405 |
| 149 | LG:332176.3:2000SEP08 | 526662H1 | 124 | 307 |
| 149 | LG:332176.3:2000SEP08 | 2856544H1 | 133 | 24,5 |
| 149 | LG:332176.3:2000SEP08 | 7247177H1 | 161 | 539 |
| 150 | LG:994938.1:2000SEP08 | 959442H1 | 1 | 275 |
| 150 | LG:994938.1:2000SEP08 | 960385H1 | 1 | 184 |
| 150 | LG:994938.1:2000SEP08 | 959442R6 | 1 | 366 |
| 150 | LG:994938.1:2000SEP08 | 3387872T8 | 185 | 740 |
| 150 | LG:994938.1:2000SEP08 | g2934522 | 342 | 630 |
| 150 | LG:994938.1:2000SEP08 | 6325528H1 | 346 | 538 |
| 150 | LG:994938.1:2000SEP08 | 407022H1 | 371 | 614 |
| 150 | LG:994938.1:2000SEP08 | 960385T6 | 423 | 772 |
| 150 | LG:994938.1:2000SEP08 | g2242138 | 425 | 810 |
| 150 | LG:994938.1:2000SEP08 | 959442T6 | 431 | 761 |
| 150 | LG:994938.1:2000SEP08 | g2969314 | 445 | 541 |
| 151 | LG:982800.1:2000SEP08 | 3118103H1 | 2147 | 2437 |
| 151 | LG:982800.1:2000SEP08 | 6020943H1 | 2062 | 2430 |
| 151 | LG:982800.1:2000SEP08 | 3219963H1 | 2062 | 2228 |
| 151 | LG:982800.1:2000SEP08 | g2540261 | 2063 | 2448 |
| 151 | LG:982800.1:2000SEP08 | 6147820H1 | 2078 | 2363 |
| 151 | LG:982800.1:2000SEP08 | 4445313H1 | 2080 | 2266 |
| 151 | LG:982800.1:2000SEP08 | 955100T2 | 2090 | 2570 |
| 151 | LG:982800.1:2000SEP08 | 955100R1 | 2090 | 2453 |
| 151 | LG:982800.1:2000SEP08 | 955100H1 | 2090 | 2358 |
| 151 | LG:982800.1:2000SEP08 | 3814175T6 | 2095 | 2570 |
| 151 | LG:982800.1:2000SEP08 | g2011857 | 2103 | 2393 |
| 151 | LG:982800.1:2000SEP08 | 2875837H1 | 2116 | 2394 |
| 151 | LG:982800.1:2000SEP08 | g1849425 | 2131 | 2424 |
| 151 | LG:982800.1:2000SEP08 | 4337528T6 | 2144 | 2581 |
| 151 | LG:982800.1:2000SEP08 | 3118103F6 | 2146 | 2582 |
| 151 | LG:982800.1:2000SEP08 | 822341R1 | 2189 | 2449 |
| 151 | LG:982800.1:2000SEP08 | g3086258 | 2215 | 2609 |
| 151 | LG:982800.1:2000SEP08 | 2101604T6 | 2217 | 2565 |
| 151 | LG:982800.1:2000SEP08 | 636591H1 | 2217 | 2470 |
| 151 | LG:982800.1:2000SEP08 | g2809907 | 2239 | 2430 |
| 151 | LG:982800.1:2000SEP08 | 496959H1 | 2271 | 2449 |
| 151 | LG:982800.1:2000SEP08 | g1927355 | 2303 | 2606 |
| 151 | LG:982800.1:2000SEP08 | 406063H1 | 2316 | 2543 |
| 151 | LG:982800.1:2000SEP08 7335618H1 | 2331 | 2616 | |
| 151 | LG:982800.1:2000SEP08 | g4329276 | 2347 | 2613 |
| 151 | LG:982800.1:2000SEP08 | 630611H1 | 2382 | 2607 |
| 151 | LG:982800.1:2000SEP08 | 904646R2 | 2397 | 2601 |
| 151 | LG:982800.1:2000SEP08 | 904646H1 | 2398 | 2608 |
| 151 | LG:982800.1:2000SEP08 | 6870517H1 | 1296 | 1814 |
| 151 | LG:982800.1:2000SEP08 | 3814709H1 | 1477 | 1746 |
| 151 | LG:982800.1:2000SEP08 | 3814709F7 | 1498 | 1730 |
| 151 | LG:982800.1:2000SEP08 | 4556204H1 | 1561 | 1642 |
| 151 | LG:982800.1:2000SEP08 | 4551845F6 | 1561 | 1744 |
| 151 | LG:982800.1:2000SEP08 | 4557749H1 | 1561 | 1744 |
| 151 | LG:982800.1:2000SEP08 | 4556204F8 | 1561 | 2151 |
| 151 | LG:982800.1:2000SEP08 | 4557749F8 | 1580 | 2139 |
| 151 | LG:982800.1:2000SEP08 | 2101604R6 | 2028 | 2532 |
| 151 | LG:982800.1:2000SEP08 | 2101604H1 | 2028 | 2294 |
| 151 | LG:982800.1:2000SEP08 | g2016400 | 2051 | 2147 |
| 151 | LG:982800.1:2000SEP08 | 804149H1 | 2058 | 2162 |
| 151 | LG:982800.1:2000SEP08 | 3485587H1 | 2062 | 2165 |
| 151 | LG:982800.1:2000SEP08 | 3999943H1 | 2062 | 2229 |
| 151 | LG:982800.1:2000SEP08 | 4337528F6 | 583 | 991 |
| 151 | LG:982800.1:2000SEP08 | 4337528H1 | 583 | 841 |
| 151 | LG:982800.1:2000SEP08 | 2790452H2 | 853 | 1006 |
| 151 | LG:982800.1:2000SEP08 | 2792488H1 | 853 | 1139 |
| 151 | LG:982800.1:2000SEP08 | 2792488F6 | 853 | 1240 |
| 151 | LG:982800.1:2000SEP08 | 7269729H1 | 1056 | 1608 |
| 151 | LG:982800.1:2000SEP08 | g1802842 | 1156 | 1515 |
| 151 | LG:982800.1:2000SEP08 | 5734055H1 | 1230 | 1497 |
| 151 | LG:982800.1:2000SEP08 | 7654174H1 | 356 | 465 |
| 151 | LG:982800.1:2000SEP08 | 7697207J 1 | 399 | 886 |
| 151 | LG:982800.1:2000SEP08 | 7697207H1 | 399 | 860 |
| 151 | LG:982800.1:2000SEP08 | 7608868J1 | 407 | 762 |
| 151 | LG:982800.1:2000SEP08 | 4265753H1 | 513 | 695 |
| 151 | LG:982800.1:2000SEP08 | 2792488T6 | 2157 | 2565 |
| 151 | LG:982800.1:2000SEP08 | 632101H1 | 2161 | 2424 |
| 151 | LG:982800.1:2000SEP08 | 3479107H1 | 2175 | 2356 |
| 151 | LG:982800.1:2000SEP08 | 822341H1 | 2189 | 2412 |
| 151 | LG:982800.1:2000SEP08 | 505559H1 | 2150 | 2362 |
| 151 | LG:982800.1:2000SEP08 | 7609838J1 | 42 | 593 |
| 151 | LG:982800.1:2000SEP08 | 761 7315J1 | 307 | 888 |
| 151 | LG:982800.1:2000SEP08 | 7617315H1 | 1 | 258 |
| 151 | LG:982800.1:2000SEP08 | 4841542H1 | 12 | 274 |
| 151 | LG:982800.1:2000SEP08 | g5632630 | 2501 | 2607 |
| 151 | LG:982800.1:2000SEP08 | g3961700 | 2503 | 2607 |
| 151 | LG:982800.1:2000SEP08 | 4383061H1 | 2412 | 2583 |
| 151 | LG:982800.1:2000SEP08 | 2130512H1 | 2412 | 2606 |
| 152 | LG:977850.7:2000SEP08 | 6832831H1 | 1 | 270 |
| 152 | LG:977850.7:2000SEP08 | 6832952H1 | 2 | 270 |
| 153 | LG:234748.2:2000SEP08 | 3810352H1 | 1966 | 2273 |
| 153 | LG:234748.2:2000SEP08 | 5878092H1 | 2081 | 2258 |
| 153 | LG:234748.2:2000SEP08 | g1696827 | 1956 | 2256 |
| 153 | LG:234748.2:2000SEP08 | g4901064 | 1794 | 2244 |
| 153 | LG:234748.2:2000SEP08 | 5016719H1 | 1953 | 2241 |
| 153 | LG:234748.2:2000SEP08 | 6752013H1 | 1629 | 2144 |
| 153 | LG:234748.2:2000SEP08 | 6314130H1 | 1611 | 2120 |
| 153 | LG:234748.2:2000SEP08 | g3173456 | 1782 | 2119 |
| 153 | LG:234748.2:2000SEP08 | 6781 18H1 | 1823 | 2085 |
| 153 | LG:234748.2:2000SEP08 | 2044168H1 | 1756 | 2014 |
| 153 | LG:234748.2:2000SEP08 | 5267795H1 | 1758 | 1977 |
| 153 | LG:234748.2:2000SEP08 | 1505116F6 | 1546 | 1947 |
| 153 | LG:234748.2:2000SEP08 | 37401 14H1 | 1651 | 1940 |
| 153 | LG:234748.2:2000SEP08 | 2047028H1 | 1025 | 1286 |
| 153 | LG:234748.2:2000SEP08 | 1413260H1 | 1022 | 1267 |
| 153 | LG:234748.2:2000SEP08 | g3886794 | 890 | 1258 |
| 153 | LG:234748.2:2000SEP08 | 2047028F6 | 938 | 1286 |
| 153 | LG:234748.2:2000SEP08 | g1481863 | 868 | 1308 |
| 153 | LG:234748.2:2000SEP08 | 4922824H1 | 1032 | 1296 |
| 153 | LG:234748.2:2000SEP08 | 2284804H1 | 1177 | 1407 |
| 153 | LG:234748.2:2000SEP08 | 2284804R6 | 1017 | 1407 |
| 153 | LG:234748.2:2000SEP08 | 5833129H1 | 1126 | 1391 |
| 153 | LG:234748.2:2000SEP08 | 3906375H1 | 1201 | 1379 |
| 153 | LG:234748.2:2000SEP08 | 4440961H1 | 1055 | 1342 |
| 153 | LG:234748.2:2000SEP08 | 5387151T6 | 1030 | 1323 |
| 153 | LG:234748.2:2000SEP08 | g6658351 | 866 | 1324 |
| 153 | LG:234748.2:2000SEP08 | 3320618F6 | 503 | 1049 |
| 153 | LG:234748.2:2000SEP08 | 3716611F6 | 520 | 1007 |
| 153 | LG:234748.2:2000SEP08 | 4178471H1 | 924 | 998 |
| 153 | LG:234748.2:2000SEP08 | 3270832H1 | 741 | 990 |
| 153 | LG:234748.2:2000SEP08 | g2819681 | 533 | 959 |
| 153 | LG:234748.2:2000SEP08 | g3870024 | 513 | 956 |
| 153 | LG:234748.2:2000SEP08 | g2112072 | 617 | 954 |
| 153 | LG:234748.2:2000SEP08 | 95664450 | 549 | 950 |
| 153 | LG:234748.2:2000SEP08 | 2914043H1 | 648 | 917 |
| 153 | LG:234748.2:2000SEP08 | 3669215T6 | 531 | 909 |
| 153 | LG:234748.2:2000SEP08 | 3093657H1 | 658 | 902 |
| 153 | LG:234748.2:2000SEP08 | 4316137H1 | 550 | 823 |
| 153 | LG:234748.2:2000SEP08 | 3533183H1 | 626 | 818 |
| 153 | LG:234748.2:2000SEP08 | 3716611H1 | 521 | 812 |
| 153 | LG:234748.2:2000SEP08 | 3792070H1 | 508 | 790 |
| 153 | LG:234748.2:2000SEP08 | 3705837F6 | 355 | 760 |
| 153 | LG:234748.2:2000SEP08 | 6444785H1 | 572 | 1112 |
| 153 | LG:234748.2:2000SEP08 | 3693858H1 | 800 | 1101 |
| 153 | LG:234748.2:2000SEP08 | 3463580H1 | 843 | 1101 |
| 153 | LG:234748.2:2000SEP08 | 6731509H1 | 604 | 1 166 |
| 153 | LG:234748.2:2000SEP08 | 371661 1T6 | 608 | 1129 |
| 153 | LG:234748.2:2000SEP08 | g5431286 | 728 | 1125 |
| 153 | LG:234748.2:2000SEP08 | 93058557 | 948 | 1125 |
| 153 | LG:234748.2:2000SEP08 | 6444794H1 | 626 | 1112 |
| 153 | LG:234748.2:2000SEP08 | 2293674H1 | 1250 | 1473 |
| 153 | LG:234748.2:2000SEP08 | 3935051H1 | 1163 | 1446 |
| 153 | LG:234748.2:2000SEP08 | 6044392H1 | 759 | 1251 |
| 153 | LG:234748.2:2000SEP08 | 6044534H1 | 730 | 1251 |
| 153 | LG:234748.2:2000SEP08 | 5087050H1 | 1321 | 1503 |
| 153 | LG:234748.2:2000SEP08 | 2743371F6 | 2356 | 2826 |
| 153 | LG:234748.2:2000SEP08 | g5438479 | 2180 | 2633 |
| 153 | LG:234748.2:2000SEP08 | g6992169 | 2256 | 2632 |
| 153 | LG:234748.2:2000SEP08 | g4740416 | 2388 | 2629 |
| 153 | LG:234748.2:2000SEP08 | 94600044 | 2230 | 2626 |
| 153 | LG:234748.2:2000SEP08 | g6992693 | 2346 | 2626 |
| 153 | LG:234748.2:2000SEP08 | 1505116T6 | 2043 | 2589 |
| 153 | LG:234748.2:2000SEP08 | 2743371H1 | 2356 | 2580 |
| 153 | LG:234748.2:2000SEP08 | g1481317 | 2164 | 2559 |
| 153 | LG:234748.2:2000SEP08 | g1523156 | 2169 | 2560 |
| 153 | LG:234748.2:2000SEP08 | 95426194 | 2139 | 2552 |
| 153 | LG:234748.2:2000SEP08 | g5630258 | 2178 | 2549 |
| 153 | LG:234748.2:2000SEP08 | g5176173 | 2163 | 2549 |
| 153 | LG:234748.2:2000SEP08 | g3050489 | 2285 | 2503 |
| 153 | LG:234748.2:2000SEP08 | 3825734H1 | 2211 | 2484 |
| 153 | LG:234748.2:2000SEP08 | 4598219H1 | 2146 | 2428 |
| 153 | LG:234748.2:2000SEP08 | 4520112H1 | 2216 | 2343 |
| 153 | LG:234748.2:2000SEP08 | 93174864 | 2007 | 2343 |
| 153 | LG:234748.2:2000SEP08 | 4309556H1 | 1990 | 2308 |
| 153 | LG:234748.2:2000SEP08 | 3320618T6 | 1712 | 2301 |
| 153 | LG:234748.2:2000SEP08 | 5150811F6 | 24 | 300 |
| 153 | LG:234748.2:2000SEP08 | 5150811H1 | 25 | 297 |
| 153 | LG:234748.2:20003EP08 | 6044392J1 | 1 | 474 |
| 153 | LG:234748.2:2000SEP08 | 6044534J1 | 1 | 465 |
| 153 | LG:234748.2:2000SEP08 | g1696922 | 114 | 441 |
| 153 | LG:234748.2:2000SEP08 | g3849721 | 5 | 392 |
| 153 | LG:234748.2:2000SEP08 | g4606643 | 98 | 386 |
| 153 | LG:234748.2:2000SEP08 | 5411009H1 | 1618 | 1874 |
| 153 | LG:234748.2:2000SEP08 | 042271H1 | 1660 | 1862 |
| 153 | LG:234748.2:2000SEP08 | 418644T6 | 1358 | 1853 |
| 153 | LG:234748.2:2000SEP08 | 3705837T6 | 1220 | 1841 |
| 153 | LG:234748.2:2000SEP08 | 1505116H1 | 1546 | 1765 |
| 153 | LG:234748.2:2000SEP08 | 418644R6 | 1294 | 1753 |
| 153 | LG:234748.2:2000SEP08 | 2750637H1 | 1449 | 1716 |
| 153 | LG:234748.2:2000SEP08 | 029958H1 | 1328 | 1571 |
| 153 | LG:234748.2:2000SEP08 | 5909796H1 | 1264 | 1557 |
| 153 | LG:234748.2:2000SEP08 | 418644H1 | 1294 | 1544 |
| 153 | LG:234748.2:2000SEP08 | 2896217H1 | 1242 | 1535 |
| 153 | LG:234748.2:2000SEP08 | 1582705H1 | 1344 | 1533 |
| 153 | LG:234748.2:2000SEP08 | 413712H1 | 1294 | 1527 |
| 153 | LG:234748.2:2000SEP08 | 7694715J1 | 1008 | 1523 |
| 153 | LG:234748.2:2000SEP08 | 3155457H1 | 1228 | 1518 |
| 153 | LG:234748.2:2000SEP08 | 412062H1 | 1294 | 1508 |
| 153 | LG:234748.2:2000SEP08 | 3320618H1 | 504 | 759 |
| 153 | LG:234748.2:2000SEP08 | 7694715H1 | 301 | 718 |
| 153 | LG:234748.2:2000SEP08 | g5633027 | 254 | 708 |
| 153 | LG:234748.2:2000SEP08 | g2725769 | 279 | 705 |
| 153 | LG:234748.2:2000SEP08 | g3147355 | 280 | 705 |
| 153 | LG:234748.2:2000SEP08 | 4798887H1 | 428 | 685 |
| 153 | LG:234748.2:2000SEP08 | 2768878T6 | 94 | 666 |
| 153 | LG:234748.2:2000SEP08 | 3705837H1 | 356 | 639 |
| 153 | LG:234748.2:2000SEP08 | 4715141H1 | 281 | 563 |
| 153 | LG:234748.2:2000SEP08 | g2737814 | 1629 | 1900 |
| 153 | LG:234748.2:2000SEP08 | g6746116 | 1480 | 1894 |
| 153 | LG:234748.2:2000SEP08 | g5109422 | 1480 | 1891 |
| 153 | LG:234748.2:2000SEP08 | g5444199 | 1441 | 1891 |
| 153 | LG:234748.2:2000SEP08 | g4852038 | 1433 | 1891 |
| 154 | LG:306284.1:2000SEP08 | 5972128H1 | 167 | 621 |
| 154 | LG:306284.1:2000SEP08 | 7091536H1 | 1 | 511 |
| 154 | LG:306284.1:2000SEP08 | 7190549H1 | 1 | 488 |
| 154 | LG:306284.1:2000SEP08 | g2882959 | 58 | 298 |
| 155 | LI:333170.3:2000SEP08 | g5837651 | 208 | 614 |
| 155 | LI:333170.3:2000SEP08 | g3742832 | 264 | 617 |
| 155 | LI:333170.3:2000SEP08 | 2004907R6 | 58 | 580 |
| 155 | LI:333170.3:2000SEP08 | 2004907T6 | 245 | 576 |
| 155 | LI:333170.3:2000SEP08 | g5363197 | 166 | 618 |
| 155 | LI:333170.3:2000SEP08 | g5445884 | 247 | 618 |
| 155 | LI:333170.3:2000SEP08 | g5637698 | 360 | 618 |
| 155 | LI:333170.3:2000SEP08 | g2805844 | 246 | 618 |
| 155 | LI:333170.3:2000SEP08 | g5753838 | 157 | 618 |
| 155 | LI:333170.3:2000SEP08 | g3038739 | 476 | 618 |
| 155 | LI:333170.3:2000SEP08 | g3017051 | 280 | 623 |
| 155 | LI:333170.3:2000SEP08 | g5445633 | 156 | 618 |
| 155 | LI:333170.3:2000SEP08 | g6700310 | 210 | 617 |
| 155 | LI:333170.3:2000SEP08 | g3764176 | 181 | 617 |
| 155 | LI:333170.3:2000SEP08 | g5767190 | 159 | 617 |
| 155 | LI:333170.3:2000SEP08 | 2006067H1 | 565 | 617 |
| 155 | LI:333170.3:2000SEP08 | g5540927 | 480 | 617 |
| 155 | LI:333170.3:2000SEP08 | g6569558 | 177 | 617 |
| 155 | LI:333170.3:2000SEP08 | g5755109 | 233 | 617 |
| 155 | LI:333170.3:2000SEP08 | g5809821 | 165 | 617 |
| 155 | LI:333170.3:2000SEP08 | g5765179 | 304 | 617 |
| 155 | LI:333170.3:2000SEP08 | g5766178 | 231 | 617 |
| 155 | LI:333170.3:2000SEP08 | g3058091 | 310 | 617 |
| 155 | LI:333170.3:2000SEP08 | g4685400 | 298 | 617 |
| 155 | LI:333170.3:2000SEP08 | g3755589 | 294 | 617 |
| 155 | LI:333170.3:2000SEP08 | g5449513 | 303 | 617 |
| 155 | LI:333170.3:2000SEP08 | g2841283 | 399 | 621 |
| 155 | LI:333170.3:2000SEP08 | 8032449J1 | 1 | 613 |
| 155 | LI:333170.3:2000SEP08 | 8032455J1 | 6 | 617 |
| 155 | LI:333170.3:2000SEP08 | 2013917R6 | 248 | 571 |
| 155 | LI:333170.3:2000SEP08 | 2013917T6 | 248 | 571 |
| 155 | LI:333170.3:2000SEP08 | 8032751J1 | 10 | 535 |
| 155 | LI:333170.3:2000SEP08 | g4630393 | 180 | 623 |
| 155 | LI:333170.3:2000SEP08 | g5445501 | 360 | 623 |
| 155 | LI:333170.3:2000SEP08 | g6451383 | 150 | 623 |
| 155 | LI:333170.3:2000SEP08 | g4737015 | 179 | 622 |
| 155 | LI:333170.3:2000SEP08 | 8032410J1 | 6 | 621 |
| 155 | LI:333170.3:2000SEP08 | g4078257 | 176 | 619 |
| 155 | LI:333170.3:2000SEP08 | g6462728 | 160 | 620 |
| 155 | LI:333170.3:2000SEP08 | g5766955 | 157 | 619 |
| 155 | LI:333170.3:2000SEP08 | 94620210 | 224 | 618 |
| 155 | LI:333170.3:2000SEP08 | g5362872 | 167 | 618 |
| 155 | LI:333170.3:2000SEP08 | g5638645 | 241 | 618 |
| 155 | LI:333170.3:2000SEP08 | 6244016H1 | 1 | 478 |
| 155 | LI:333170.3:2000SEP08 | 2013917H1 | 248 | 457 |
| 155 | LI:333170.3:2000SEP08 | 2004907H1 | 58 | 288 |
| 155 | LI:333170.3:2000SEP08 | 301440H1 | 56 | 264 |
| 155 | LI:333170.3:2000SEP08 | 2005286H1 | 93 | 257 |
| 156 | LI:336685.2:2000SEP08 | 7612160J1 | 604 | 1140 |
| 156 | LI:336685.2:2000SEP08 | 7585885H2 | 453 | 1114 |
| 156 | LI:336685.2:2000SEP08 | 7330357H1 | 424 | 933 |
| 156 | LI:336685.2:2000SEP08 | 7612160H1 | 246 | 838 |
| 156 | LI:336685.2:2000SEP08 | g3844561 | 388 | 780 |
| 156 | LI:336685.2:2000SEP08 | 91422428 | 436 | 777 |
| 156 | LI:336685.2:2000SEP08 | g2702908 | 347 | 777 |
| 156 | LI:336685.2:2000SEP08 | g3751213 | 82 | 472 |
| 156 | LI:336685.2:2000SEP08 | g3039675 | 93 | 472 |
| 156 | LI:336685.2:2000SEP08 | g1422427 | 85 | 456 |
| 156 | LI:336685.2:2000SEP08 | 3167506H1 | 1 | 292 |
| 157 | LI:279013.5:2000SEP08 | 70598934V1 | 707 | 1131 |
| 157 | LI:279013.5:2000SEP08 | 70595538V1 | 841 | 1098 |
| 157 | LI:279013.5:2000SEP08 | 70601648V1 | 531 | 912 |
| 157 | LI:279013.5:2000SEP08 | 70600435V1 | 537 | 1137 |
| 157 | LI:279013.5:2000SEP08 | 70600920V1 | 545 | 1103 |
| 157 | LI:279013.5:2000SEP08 | 70600178V1 | 548 | 988 |
| 157 | LI:279013.5:2000SEP08 | 70598102V1 | 554 | 1073 |
| 157 | LI:279013.5:2000SEP08 | 70602680V1 | 562 | 1161 |
| 157 | LI:279013.5:2000SEP08 | 70601624V1 | 563 | 1131 |
| 157 | LI:279013.5:2000SEP08 | 70598886V1 | 603 | 1087 |
| 157 | LI:279013.5:2000SEP08 | 70598993V1 | 619 | 1089 |
| 157 | LI:279013.5:2000SEP08 | 70600050V1 | 616 | 1138 |
| 157 | LI:279013.5:2000SEP08 | 70601863V1 | 618 | 1134 |
| 157 | LI:279013.5:2000SEP08 | 70601661 V1 | 615 | 1033 |
| 157 | LI:279013.5:2000SEP08 | 70599912V1 | 666 | 1215 |
| 157 | LI:279013.5:2000SEP08 | 70598696V1 | 683 | 1067 |
| 157 | LI:279013.5:2000SEP08 | 70601955V1 | 696 | 1131 |
| 157 | LI:279013.5:2000SEP08 | 70600352V1 | 1 | 283 |
| 157 | LI:279013.5:2000SEP08 | 70599180V1 | 1 | 440 |
| 157 | LI:279013.5:2000SEP08 | 70600806V1 | 1 | 76 |
| 157 | LI:279013.5:2000SEP08 | 208381 1F6 | 1 | 455 |
| 157 | LI:279013.5:2000SEP08 | 70611716V1 | 1 | 219 |
| 157 | LI:279013.5:2000SEP08 | 70597203V1 | 1 | 454 |
| 157 | LI:279013.5:2000SEP08 | 70597842V1 | 1 | 234 |
| 157 | LI:279013.5:2000SEP08 | 70597537V1 | 44 | 737 |
| 157 | LI:279013.5:2000SEP08 | 70598005V1 | 275 | 459 |
| 157 | LI:279013.5:2000SEP08 | 70602584V1 | 279 | 459 |
| 157 | LI:279013.5:2000SEP08 | 70600575V1 | 280 | 860 |
| 157 | LI:279013.5:2000SEP08 | 70599593V1 | 312 | 816 |
| 157 | LI:279013.5:2000SEP08 | 70598759V1 | 302 | 617 |
| 157 | LI:279013.5:2000SEP08 | 70594144V1 | 317 | 625 |
| 157 | LI:279013.5:2000SEP08 | 70598864V 1 | 321 | 808 |
| 157 | LI:279013.5:2000SEP08 | 70599792V1 | 404 | 989 |
| 157 | LI:279013.5:2000SEP08 | 70598913V1 | 419 | 901 |
| 157 | LI:279013.5:2000SEP08 | 70600311V1 | 484 | 916 |
| 157 | LI:279013.5:2000SEP08 | 208381 1T6 | 496 | 733 |
| 157 | LI:279013.5:2000SEP08 | 70599521 V1 | 1 | 404 |
| 157 | LI:279013.5:2000SEP08 | 70598407V1 | 1 | 162 |
| 157 | LI:279013.5:2000SEP08 | 70597887V1 | 1 | 650 |
| 157 | LI:279013.5:2000SEP08 | 70611227V1 | 904 | 1068 |
| 157 | LI:279013.5:2000SEP08 | 70612859V1 | 928 | 1087 |
| 158 | LI:1037075.1:2000SEP08 | 7413090H1 | 84 | 687 |
| 158 | LI:1037075.1:2000SEP08 | 7413080H1 | 102 | 687 |
| 158 | LI:1037075.1:2000SEP08 | 5758628T8 | 1 | 379 |
| 158 | LI:1037075.1:2000SEP08 | 5758628H1 | 2 | 317 |
| 158 | LI:1037075.1:2000SEP08 | 5758628F8 | 117 | 301 |
| 159 | LI:1073403.1:2000SEP08 | 6792032T8 | 10 | 226 |
| 159 | LI:1073403.1:2000SEP08 | 6795549H1 | 54 | 327 |
| 159 | LI:1073403.1:2000SEP08 | 6795549F8 | 54 | 327 |
| 159 | LI:1073403.1:2000SEP08 | 6794588F8 | 69 | 181 |
| 159 | LI:1073403.1:2000SEP08 | 6794588H1 | 1 | 329 |
| 159 | LI:1073403.1:2000SEP08 | 6793290H1 | 1 | 327 |
| 159 | LI:1073403.1:2000SEP08 | 6792032H1 | 10 | 329 |
| 159 | LI:1073403.1:2000SEP08 | 6792032F8 | 10 | 329 |
| 160 | LI:1075296.1:2000SEP08 | 6798109H1 | 1 | 487 |
| 160 | LI:1075296.1:2000SEP08 | 6791605F8 | 1 | 616 |
| 160 | LI:1075296.1:2000SEP08 | 6798109F8 | 1 | 630 |
| 160 | LI:1075296.1:2000SEP08 | 6794209H1 | 1 | 551 |
| 160 | LI:1075296.1:2000SEP08 | 6791605H1 | 10 | 411 |
| 160 | LI:1075296.1:2000SEP08 | 6791605T8 | 86 | 740 |
| 160 | LI:1075296.1:2000SEP08 | 6794209T8 | 89 | 699 |
| 160 | LI:1075296.1:2000SEP08 | 6798109T8 | 118 | 681 |
| 161 | LI:1085501.1:2000SEP08 | 5302896F8 | 1 | 556 |
| 161 | LI:1085501.1:2000SEP08 | 5321660F9 | 1 | 572 |
| 161 | LI:1085501.1:2000SEP08 | 5302896T9 | 227 | 783 |
| 162 | LI:1086181.1:2000SEP08 | 6791828F8 | 1 | 692 |
| 162 | LI:1086181.1:2000SEP08 | 6791828H1 | 1 | 425 |
| 162 | LI:1086181.1:2000SEP08 | 6795536T8 | 48 | 534 |
| 162 | LI:1086181.1:2000SEP08 | 6795536F8 | 48 | 586 |
| 162 | LI:1086181.1:2000SEP08 | 6794338H1 | 48 | 528 |
| 162 | LI:1086181.1:2000SEP08 | 6795536H1 | 51 | 494 |
| 162 | LI:1086181.1:2000SEP08 | 6794338F8 | 69 | 331 |
| 163 | LI:1164493.1:2000SEP08 | g6087014 | 642 | 841 |
| 163 | LI:1164493.1:2000SEP08 | g5914421 | 379 | 837 |
| 163 | LI:1164493.1:2000SEP08 | g5914276 | 501 | 835 |
| 163 | LI:1164493.1:2000SEP08 | 2573205T6 | 286 | 817 |
| 163 | LI:1164493.1:2000SEP08 | 2230675T6 | 353 | 802 |
| 163 | LI:1164493.1:2000SEP08 | 2573205R6 | 290 | 763 |
| 163 | LI:1164493.1:2000SEP08 | 2573205H1 | 290 | 537 |
| 163 | LI:1164493.1:2000SEP08 | 2675819F6 | 1 | 395 |
| 163 | LI:1164493.1:2000SEP08 | 2792163H1 | 59 | 378 |
| 163 | LI:1164493.1:2000SEP08 | 2675819H1 | 1 | 231 |
| 163 | LI:1164493.1:2000SEP08 | 2729684T6 | 1 | 231 |
| 164 | LI:1175097.1:2000SEP08 | 60131276D1 | 1 | 520 |
| 164 | LI:1175097.1:2000SEP08 | 60131277D1 | 1 | 520 |
| 164 | LI:1175097.1:2000SEP08 | 60131346D1 | 1 | 474 |
| 164 | LI:1175097.1:2000SEP08 | 60131283D1 | 1 | 523 |
| 164 | LI:1175097.1:2000SEP08 | g5109275 | 66 | 520 |
| 164 | LI:1175097.1:2000SEP08 | 990949R6 | 142 | 520 |
| 164 | LI:1175097.1:2000SEP08 | 990949T6 | 143 | 480 |
| 164 | LI:1175097.1:2000SEP08 | 990949H1 | 142 | 438 |
| 164 | LI:1175097.1:2000SEP08 | g1975314 | 233 | 529 |
| 164 | LI:1175097.1:2000SEP08 | 3081595F6 | 397 | 642 |
| 164 | LI:1175097.1:2000SEP08 | 3081595H1 | 398 | 694 |
| 165 | LI:1092948.1:2000SEP08 | 4760775H1 | 41 | 331 |
| 165 | LI:1092948.1:2000SEP08 | 7330786H2 | 103 | 415 |
| 165 | LI:1092948.1:2000SEP08 | 5509447F6 | 131 | 453 |
| 165 | LI:1092948.1:2000SEP08 | 4001814H1 | 1 | 297 |
| 165 | LI:1092948.1:2000SEP08 | 5509447H1 | 131 | 217 |
| 165 | LI:1092948.1:2000SEP08 | 3808866H1 | 7 | 305 |
| 165 | LI:1092948.1:2000SEP08 | 5428960F8 | 8 | 332 |
| 165 | LI:1092948.1:2000SEP08 | 5428960T8 | 8 | 263 |
| 165 | LI:1092948.1:2000SEP08 | 5428960H1 | 8 | 250 |
| 165 | LI:1092948.1:2000SEP08 | g2159669 | 10 | 220 |
| 165 | LI:1092948.1:2000SEP08 | 3808866F8 | 7 | 463 |
| 165 | LI:1092948.1:2000SEP08 | 1525513H1 | 9 | 225 |
| 165 | LI:1092948.1:2000SEP08 | 2889208F6 | 8 | 343 |
| 165 | LI:1092948.1:2000SEP08 | 2889208H1 | 8 | 282 |
| 165 | LI:1092948.1:2000SEP08 | 7264977H1 | 19 | 611 |
| 165 | LI:1092948.1:2000SEP08 | 3016535H1 | 23 | 307 |
| 165 | LI:1092948.1:2000SEP08 | 4760775F6 | 40 | 613 |
| 166 | LI:380378.2:2000SEP08 | 6052233R8 | 22 | 497 |
| 166 | LI:380378.2:2000SEP08 | 6861022H1 | 138 | 218 |
| 166 | LI:380378.2:2000SEP08 | 6052233H1 | 1 | 502 |
| 166 | LI:380378.2:2000SEP08 | 6052233J1 | 1 | 497 |
| 166 | LI:380378.2:2000SEP08 | 6052233F8 | 1 | 497 |
| 167 | LI:1029674.1:2000SEP08 | g6716886 | 1321 | 1562 |
| 167 | LI:1029674.1:2000SEP08 | 6763379J1 | 1073 | 1551 |
| 167 | LI:1029674.1:2000SEP08 | 6768071J1 | 1117 | 1552 |
| 167 | LI:1029674.1:2000SEP08 | g7039790 | 1343 | 1522 |
| 167 | LI:1029674.1:2000SEP08 | 6803363J1 | 634 | 1235 |
| 167 | LI:1029674.1:2000SEP08 | 6763379H1 | 221 | 774 |
| 167 | LI:1029674.1:2000SEP08 | 6803363H1 | 222 | 613 |
| 167 | LI:1029674.1:2000SEP08 | 6768071H1 | 1 | 561 |
| 168 | LI:2048601.3:2000SEP08 | 71558389V1 | 1 | 391 |
| 168 | LI:2048601.3:2000SEP08 | 71558424V1 | 1 | 391 |
| 169 | LI:1186208.1:2000SEP08 | 4294932F6 | 35 | 302 |
| 169 | LI:1186208.1:2000SEP08 | 1899877H1 | 67 | 291 |
| 169 | LI:1186208.1:2000SEP08 | 2882961H1 | 40 | 298 |
| 169 | LI:1186208.1:2000SEP08 | 1315261H1 | 37 | 159 |
| 169 | LI:1186208.1:2000SEP08 | 5120882H1 | 29 | 291 |
| 169 | LI:1186208.1:2000SEP08 | 91959316 | 67 | 383 |
| 169 | LI:1186208.1:2000SEP08 | 1313874H1 | 37 | 159 |
| 169 | LI:1186208.1:2000SEP08 | 4399221H1 | 48 | 260 |
| 169 | LI:1186208.1:2000SEP08 | g2401851 | 35 | 267 |
| 169 | LI:1186208.1:2000SEP08 | 1257339H1 | 46 | 267 |
| 169 | LI:1186208.1:2000SEP08 | 7976538H1 | 1 | 562 |
| 169 | LI:1186208.1:2000SEP08 | 5120882F6 | 29 | 379 |
| 169 | LI:1186208.1:2000SEP08 | 014670H1 | 34 | 314 |
| 169 | LI:1186208.1:2000SEP08 | 8099166H1 | 37 | 657 |
| 169 | LI:1186208.1:2000SEP08 | 1315261F6 | 37 | 540 |
| 169 | LI:1186208.1:2000SEP08 | 5378061H1 | 159 | 400 |
| 169 | LI:1186208.1:2000SEP08 5378061F6 | 159 | 617 | |
| 169 | LI:1186208.1:2000SEP08 | g4265183 | 206 | 667 |
| 169 | LI:1186208.1:2000SEP08 | 4881456H1 | 300 | 379 |
| 169 | LI:1186208.1:2000SEP08 | 1315261T6 | 1 | 65 |
| 170 | LI:1170753.1:2000SEP08 | 6200892T8 | 1 | 296 |
| 170 | LI:1170753.1:2000SEP08 | 5406931H1 | 256 | 392 |
| 170 | LI:1170753.1:2000SEP08 | g2264772 | 1 | 331 |
| 170 | LI:1170753.1:2000SEP08 | 6200319T8 | 1 | 297 |
| 170 | LI:1170753.1:2000SEP08 | 6200892H1 | 1 | 420 |
| 170 | LI:1170753.1:2000SEP08 | 6200792H1 | 1 | 413 |
| 170 | LI:1170753.1:2000SEP08 | 6200319F8 | 12 | 383 |
| 170 | LI:1170753.1:2000SEP08 | 5406931F8 | 264 | 393 |
| 170 | LI:1170753.1:2000SEP08 | 5406931F7 | 273 | 396 |
| 170 | LI:1170753.1:2000SEP08 | 6200892F8 | 4 | 396 |
| 171 | LI:1180908.1:2000SEP08 | g3228157 | 1 | 148 |
| 171 | LI:1180908.1:2000SEP08 | 123936H1 | 130 | 209 |
| 171 | LI:1180908.1:2000SEP08 | g1734408 | 366 | 769 |
| 171 | LI:1180908.1:2000SEP08 | 6022303H1 | 194 | 370 |
| 171 | LI:1180908.1:2000SEP08 | 3706108H1 | 147 | 414 |
| 171 | LI:1180908.1:2000SEP08 | 3630279H1 | 164 | 310 |
| 171 | LI:1180908.1:2000SEP08 | 3319137H1 | 129 | 370 |
| 171 | LI:1180908.1:2000SEP08 | 7697049J1 | 465 | 714 |
| 171 | LI:1180908.1:2000SEP08 | 7697049H1 | 476 | 714 |
| 171 | LI:1180908.1:2000SEP08 | 5283246H1 | 152 | 413 |
| 171 | LI:1180908.1:2000SEP08 | 1425317H1 | 1 | 226 |
| 171 | LI:1180908.1:2000SEP08 | 5283246F7 | 152 | 446 |
| 171 | LI:1180908.1:2000SEP08 | g2555712 | 31 | 502 |
| 171 | LI:1180908.1:2000SEP08 | 3337317H1 | 103 | 369 |
| 171 | LI:1180908.1:2000SEP08 | 2937838H1 | 112 | 377 |
| 171 | LI:1180908.1:2000SEP08 | 3562879H1 | 192 | 383 |
| 171 | LI:1180908.1:2000SEP08 | 1987023 | 199 | 489 |
| 171 | LI:1180908.1:2000SEP08 | 1256276H1 | 203 | 427 |
| 171 | LI:1180908.1:2000SEP08 | 5026847H1 | 218 | 375 |
| 171 | LI:1180908.1:2000SEP08 | 6490895R9 | 313 | 770 |
| 171 | LI:1180908.1:2000SEP08 | 4184474H1 | 327 | 512 |
| 171 | LI:1180908.1:2000SEP08 | 5564064H1 | 549 | 754 |
| 171 | LI:1180908.1:2000SEP08 | 1425317R6 | 1 | 394 |
| 171 | LI:1180908.1:2000SEP08 | 026841H1 | 1 | 192 |
| 172 | LI:1182900.2:2000SEP08 | 6928688F8 | 378 | 597 |
| 172 | LI:1182900.2:2000SEP08 | 6928688R8 | 371 | 597 |
| 172 | LI:1182900.2:2000SEP08 | 3666231T6 | 158 | 583 |
| 172 | LI:1182900.2:2000SEP08 | g4069988 | 1 | 455 |
| 172 | LI:1182900.2:2000SEP08 | g4069986 | 18 | 454 |
| 173 | LI:1169548.2:2000SEP08 | 7074830H1 | 1 | 515 |
| 174 | LI:1039974.1:2000SEP08 | 869881H1 | 595 | 859 |
| 174 | LI:1039974.1:2000SEP08 | 71151231V1 | 596 | 1063 |
| 174 | LI:1039974.1:2000SEP08 | g5678261 | 1200 | 1665 |
| 174 | LI:1039974.1:2000SEP08 | g3755479 | 1345 | 1665 |
| 174 | LI:1039974.1:2000SEP08 | 6775488H1 | 1160 | 1576 |
| 174 | LI:1039974.1:2000SEP08 | 6775710H1 | 1160 | 1576 |
| 174 | LI:1039974.1:2000SEP08 | 6775488J1 | 1160 | 1576 |
| 174 | LI:1039974.1:2000SEP08 | 6775710J1 | 1160 | 1576 |
| 174 | LI:1039974.1:2000SEP08 | 1251928F6 | 1166 | 1638 |
| 174 | LI:1039974.1:2000SEP08 | 7460336H1 | 1 | 246 |
| 174 | LI:1039974.1:2000SEP08 71153237V1 | 595 | 1070 | |
| 174 | LI:1039974.1:2000SEP08 71282025V1 | 595 | 769 | |
| 174 | LI:1039974.1:2000SEP08 | 869881R6 | 595 | 982 |
| 174 | LI:1039974.1:2000SEP08 | 7766729J2 | 1195 | 1645 |
| 174 | LI:1039974.1:2000SEP08 | 6954283H1 | 1 | 525 |
| 174 | LI:1039974.1:2000SEP08 | 3476395H1 | 12 | 282 |
| 174 | LI:1039974.1:2000SEP08 | 3081284F6 | 212 | 659 |
| 174 | LI:1039974.1:2000SEP08 | 3081284H1 | 212 | 500 |
| 174 | LI:1039974.1:2000SEP08 | 6893004J1 | 689 | 1290 |
| 174 | LI:1039974.1:2000SEP08 | 7438574H1 | 1013 | 1333 |
| 174 | LI:1039974.1:2000SEP08 | 7709152J1 | 1039 | 1598 |
| 174 | LI:1039974.1:2000SEP08 | 3423941H1 | 1085 | 1344 |
| 174 | LI:1039974.1:2000SEP08 | 3423941F6 | 1085 | 1374 |
| 174 | LI:1039974.1:2000SEP08 | 6982460H1 | 1104 | 1534 |
| 174 | LI:1039974.1:2000SEP08 | 6947579H1 | 1112 | 1620 |
| 174 | LI:1039974.1:2000SEP08 | 7763864H1 | 1 | 489 |
| 174 | LI:1039974.1:2000SEP08 | 70938370V1 | 763 | 961 |
| 174 | LI:1039974.1:2000SEP08 | 3463824F6 | 844 | 1240 |
| 174 | LI:1039974.1:2000SEP08 | 3463824H1 | 844 | 1063 |
| 174 | LI:1039974.1:2000SEP08 | 70941631 V1 | 856 | 1063 |
| 174 | LI:1039974.1:2000SEP08 | 7091561H1 | 871 | 1157 |
| 174 | LI:1039974.1:2000SEP08 | 71152516V1 | 891 | 1393 |
| 174 | LI:1039974.1:2000SEP08 | 6883174H1 | 906 | 1417 |
| 174 | LI:1039974.1:2000SEP08 | 6883129H1 | 906 | 1237 |
| 174 | LI:1039974.1:2000SEP08 | 6996726H1 | 907 | 1480 |
| 174 | LI:1039974.1:2000SEP08 | 7663341J1 | 924 | 1545 |
| 174 | LI:1039974.1:2000SEP08 | 70941275V1 | 924 | 1063 |
| 174 | LI:1039974.1:2000SEP08 | g1695282 | 710 | 919 |
| 174 | LI:1039974.1:2000SEP08 | 71152694V1 | 626 | 1063 |
| 174 | LI:1039974.1:2000SEP08 | 7091561F8 | 671 | 1149 |
| 174 | LI:1039974.1:2000SEP08 | 71151884V1 | 685 | 1063 |
| 174 | LI:1039974.1:2000SEP08 | 1251928H1 | 1166 | 1438 |
| 174 | LI:1039974.1:2000SEP08 | 7707065J1 | 947 | 1424 |
| 174 | LI:1039974.1:2000SEP08 | 3463824T7 | 976 | 1081 |
| 174 | LI:1039974.1:2000SEP08 | 7746155J1 | 993 | 1346 |
| 174 | LI:1039974.1:2000SEP08 | 71301718V1 | 647 | 1063 |
| 174 | LI:1039974.1:2000SEP08 | 7639946J2 | 416 | 1070 |
| 174 | LI:1039974.1:2000SEP08 | 7121329H1 | 490 | 909 |
| 174 | LI:1039974.1:2000SEP08 | 7735245J1 | 669 | 1255 |
| 174 | LI:1039974.1:2000SEP08 | 3337158H1 | 564 | 843 |
| 174 | LI:1039974.1:2000SEP08 | 71302652V1 | 584 | 1112 |
| 174 | LI:1039974.1:2000SEP08 | 71301474V1 | 595 | 1108 |
| 174 | LI:1039974.1:2000SEP08 | 7709152H1 | 532 | 1137 |
| 174 | LI:1039974.1:2000SEP08 | 7091561R8 | 319 | 951 |
| 174 | LI:1039974.1:2000SEP08 | 7678465H1 | 239 | 692 |
| 174 | LI:1039974.1:2000SEP08 | 5517746H1 | 346 | 566 |
| 174 | LI:1039974.1:2000SEP08 | 8036193H1 | 406 | 1033 |
| 175 | LI:1175765.2:2000SEP08 | 6985759F8 | 1 | 481 |
| 175 | LI:1175765.2:2000SEP08 | 6985759R8 | 1 | 481 |
| 176 | LI:313948.1:2000SEP08 | g4150106 | 244 | 602 |
| 176 | LI:313948.1:2000SEP08 | g2930824 | 278 | 601 |
| 176 | LI:313948.1:2000SEP08 | 8025310J1 | 1 | 593 |
| 176 | LI:313948.1:2000SEP08 | 8020685J1 | 29 | 593 |
| 176 | LI:313948.1:2000SEP08 | g5111878 | 111 | 569 |
| 176 | LI:313948.1:2000SEP08 | g4533060 | 154 | 569 |
| 177 | LI:335923.2:2000SEP08 | 6243152H1 | 1 | 565 |
| 177 | LI:335923.2:2000SEP08 | 5283710H1 | 8 | 159 |
| 177 | LI:335923.2:2000SEP08 | 5283642H1 | 8 | 201 |
| 177 | LI:335923.2:2000SEP08 | 2011071R6 | 15 | 484 |
| 177 | LI:335923.2:2000SEP08 | 2011071H1 | 15 | 221 |
| 177 | LI:335923.2:2000SEP08 | 5283929H1 | 23 | 296 |
| 177 | LI:335923.2:2000SEP08 | 5283676H1 | 23 | 285 |
| 177 | LI:335923.2:2000SEP08 | 6247161H1 | 23 | 479 |
| 177 | LI:335923.2:2000SEP08 | g2035703 | 28 | 384 |
| 177 | LI:335923.2:2000SEP08 | 2005418H1 | 168 | 357 |
| 177 | LI:335923.2:2000SEP08 | g4683784 | 245 | 728 |
| 177 | LI:335923.2:2000SEP08 | g3678673 | 308 | 738 |
| 177 | LI:335923.2:2000SEP08 | g4734389 | 311 | 735 |
| 177 | LI:335923.2:2000SEP08 | g3038675 | 646 | 737 |
| 178 | LI:345884.1:2000SEP08 | 92240240 | 1 | 315 |
| 178 | LI:345884.1:2000SEP08 | 4676488H1 | 8 | 263 |
| 178 | LI:345884.1:2000SEP08 | 4676488F6 | 7 | 392 |
| 178 | LI:345884.1:2000SEP08 | 7760811J1 | 59 | 265 |
| 178 | LI:345884.1:2000SEP08 | 94991320 | 192 | 650 |
| 178 | LI:345884.1:2000SEP08 | 4676488T6 | 214 | 620 |
| 178 | LI:345884.1:2000SEP08 | g7319648 | 465 | 557 |
| 179 | LI:417127.1:2000SEP08 | 5854850T8 | 1 | 541 |
| 179 | LI:417127.1:2000SEP08 | 5854850F8 | 1 | 556 |
| 179 | LI:417127.1:2000SEP08 | 5854850H1 | 1 | 286 |
| 180 | LI:451710.1:2000SEP08 | 5914133H1 | 1 | 282 |
| 180 | LI:451710.1:2000SEP08 | 5914133F6 | 1 | 630 |
| 180 | LI:451710.1:2000SEP08 | 5321108F9 | 1 | 537 |
| 180 | LI:451710.1:2000SEP08 | 5914133F8 | 1 | 391 |
| 180 | LI:451710.1:2000SEP08 | 5914133T6 | 52 | 609 |
| 181 | LI:406882.2:2000SEP08 | 2074401H1 | 1 | 240 |
| 181 | LI:406882.2:2000SEP08 | 7277422H1 | 1 | 572 |
| 181 | LI:406882.2:2000SEP08 | 6387643H1 | 1 | 138 |
| 181 | LI:406882.2:2000SEP08 | 5678260H1 | 2 | 262 |
| 181 | LI:406882.2:2000SEP08 | 3488977F6 | 19 | 435 |
| 181 | LI:406882.2:2000SEP08 | 3488977H1 | 19 | 214 |
| 181 | LI:406882.2:2000SEP08 | 3557611H1 | 21 | 316 |
| 181 | LI:406882.2:2000SEP08 | 6771944J1 | 110 | 686 |
| 181 | LI:406882.2:2000SEP08 | g2179336 | 168 | 373 |
| 181 | LI:406882.2:2000SEP08 | 322004H1 | 316 | 451 |
| 181 | LI:406882.2:2000SEP08 | 322004R6 | 322 | 786 |
| 181 | LI:406882.2:2000SEP08 | g1186140 | 342 | 702 |
| 181 | LI:406882.2:2000SEP08 | g2184370 | 491 | 909 |
| 181 | LI:406882.2:2000SEP08 | 91426251 | 665 | 963 |
| 181 | LI:406882.2:2000SEP08 | g2715236 | 750 | 1226 |
| 181 | LI:406882.2:2000SEP08 | 3099966T6 | 767 | 1180 |
| 181 | LI:406882.2:2000SEP08 | 3099966F6 | 774 | 1198 |
| 181 | LI:406882.2:2000SEP08 | 3099966H1 | 774 | 1087 |
| 181 | LI:406882.2:2000SEP08 | 6984563H1 | 849 | 1340 |
| 181 | LI:406882.2:2000SEP08 | g2184149 | 860 | 1226 |
| 181 | LI:406882.2:2000SEP08 | g3801693 | 863 | 1227 |
| 181 | LI:406882.2:2000SEP08 | g1153584 | 1000 | 1236 |
| 181 | LI:406882.2:2000SEP08 | 6983618H1 | 1013 | 1165 |
| 181 | LI:406882.2:2000SEP08 | 6983555H1 | 1013 | 1350 |
| 181 | LI:406882.2:2000SEP08 | g2348522 | 1012 | 1226 |
| 181 | LI:406882.2:2000SEP08 | g3841648 | 1047 | 1230 |
| 181 | LI:406882.2:2000SEP08 | g3842646 | 1049 | 1232 |
| 182 | LI:728223.1:2000SEP08 | 6269813T8 | 1 | 599 |
| 182 | LI:728223.1:2000SEP08 | 6269813F8 | 1 | 658 |
| 182 | LI:728223.1:2000SEP08 | 6269813H1 | 1 | 404 |
| 183 | LI:289783.19:2000SEP08 | 550475R1 | 800 | 1163 |
| 183 | LI:289783.19:2000SEP08 | 5512967H1 | 811 | 1037 |
| 183 | LI:289783.19:2000SEP08 | 3326063H1 | 816 | 1105 |
| 183 | LI:289783.19:2000SEP08 | 3401385H1 | 814 | 1043 |
| 183 | LI:289783.19:2000SEP08 | 777649H1 | 833 | 1046 |
| 183 | LI:289783.19:2000SEP08 | 495183H1 | 848 | 1089 |
| 183 | LI:289783.19:2000SEP08 | 2787013H1 | 878 | 1139 |
| 183 | LI:289783.19:2000SEP08 | 5432830H1 | 889 | 1108 |
| 183 | LI:289783.19:2000SEP08 | 550475H1 | 800 | 1026 |
| 183 | LI:289783.19:2000SEP08 | 7267409H1 | 688 | 1177 |
| 183 | LI:289783.19:2000SEP08 | g2537937 | 557 | 1041 |
| 183 | LI:289783.19:2000SEP08 | 60214935LI1 | 324 | 873 |
| 183 | LI:289783.19:2000SEP08 | 7084522H1 | 328 | 823 |
| 183 | LI:289783.19:2000SEP08 | 8016832J1 | 1 | 455 |
| 183 | LI:289783.19:2000SEP08 | 8016705J1 | 1 | 455 |
| 184 | LI:235255.8:2000SEP08 | 5898645H1 | 511 | 773 |
| 184 | LI:235255.8:2000SEP08 | 5901761H1 | 511 | 796 |
| 184 | LI:235255.8:2000SEP08 | 5643211H1 | 514 | 771 |
| 184 | LI:235255.8:2000SEP08 | 3188563H1 | 523 | 863 |
| 184 | LI:235255.8:2000SEP08 | 7254655H1 | 554 | 911 |
| 184 | LI:235255.8:2000SEP08 | 4569548H1 | 568 | 845 |
| 184 | LI:235255.8:2000SEP08 | 2213566F6 | 580 | 856 |
| 184 | LI:235255.8:2000SEP08 | 2224227F6 | 580 | 1019 |
| 184 | LI:235255.8:2000SEP08 | 2224227H1 | 580 | 823 |
| 184 | LI:235255.8:2000SEP08 | 2834918H1 | 592 | 839 |
| 184 | LI:235255.8:2000SEP08 | 7613084H1 | 597 | 1179 |
| 184 | LI:235255.8:2000SEP08 | 5528066H1 | 610 | 871 |
| 184 | LI:235255.8:2000SEP08 | 675898H1 | 634 | 903 |
| 184 | LI:235255.8:2000SEP08 | 70568158V1 | 660 | 1065 |
| 184 | LI:235255.8:2000SEP08 | 4625774H1 | 1 | 250 |
| 184 | LI:235255.8:2000SEP08 | 4625546H1 | 5 | 259 |
| 184 | LI:235255.8:2000SEP08 | 7203158H1 | 90 | 447 |
| 184 | LI:235255.8:2000SEP08 | 70300587D1 | 120 | 420 |
| 184 | LI:235255.8:2000SEP08 | 7051 3655D1 | 122 | 698 |
| 184 | LI:235255.8:2000SEP08 | 6883819H1 | 185 | 453 |
| 184 | LI:235255.8:2000SEP08 | 6777538J1 | 224 | 898 |
| 184 | LI:235255.8:2000SEP08 | 7680150J1 | 272 | 878 |
| 184 | LI:235255.8:2000SEP08 | 3594396H1 | 283 | 584 |
| 184 | LI:235255.8:2000SEP08 | 4383752H 1 | 285 | 541 |
| 184 | LI:235255.8:2000SEP08 | 70570626V1 | 294 | 969 |
| 184 | LI:235255.8:2000SEP08 | 55015910H1 | 310 | 726 |
| 184 | LI:235255.8:2000SEP08 | 744281H1 | 317 | 547 |
| 184 | LI:235255.8:2000SEP08 | 7619975H1 | 318 | 826 |
| 184 | LI:235255.8:2000SEP08 | 1436369F1 | 317 | 798 |
| 184 | LI:235255.8:2000SEP08 | 7619975J1 | 360 | 901 |
| 184 | LI:235255.8:2000SEP08 | 5021074H1 | 349 | 613 |
| 184 | LI:235255.8:2000SEP08 | 5020874H1 | 349 | 617 |
| 184 | LI:235255.8:2000SEP08 | 70566905V1 | 376 | 466 |
| 184 | LI:235255.8:2000SEP08 | 7688171J1 | 381 | 901 |
| 184 | LI:235255.8:2000SEP08 | 70570192V1 | 414 | 1008 |
| 184 | LI:235255.8:2000SEP08 | 70570560V1 | 417 | 983 |
| 184 | LI:235255.8:2000SEP08 | 5775092H1 | 418 | 911 |
| 184 | LI:235255.8:2000SEP08 | 4533965H1 | 453 | 706 |
| 184 | LI:235255.8:2000SEP08 | 5275166H1 | 454 | 705 |
| 184 | LI:235255.8:2000SEP08 | 236201 1H1 | 456 | 699 |
| 184 | LI:235255.8:2000SEP08 | 236201 1R6 | 456 | 796 |
| 184 | LI:235255.8:2000SEP08 | 70568674V1 | 462 | 769 |
| 184 | LI:235255.8:2000SEP08 | 6889089J1 | 462 | 914 |
| 184 | LI:235255.8:2000SEP08 | 70553659V1 | 462 | 768 |
| 184 | LI:235255.8:2000SEP08 | 70570674V1 | 462 | 768 |
| 184 | LI:235255.8:2000SEP08 | 1506551H1 | 478 | 675 |
| 184 | LI:235255.8:2000SEP08 | 70571785V1 | 496 | 729 |
| 184 | LI:235255.8:2000SEP08 | 5903079H1 | 511 | 796 |
| 184 | LI:235255.8:2000SEP08 | 5897612H1 | 511 | 821 |
| 184 | LI:235255.8:2000SEP08 | 5903174H1 | 511 | 811 |
| 184 | LI:235255.8:2000SEP08 | 3029777H1 | 679 | 970 |
| 184 | LI:235255.8:2000SEP08 | 6931063H1 | 708 | 1207 |
| 184 | LI:235255.8:2000SEP08 | 4226879H1 | 726 | 1017 |
| 184 | LI:235255.8:2000SEP08 | 7688171H1 | 736 | 1290 |
| 184 | LI:235255.8:2000SEP08 | 7606153J1 | 749 | 1198 |
| 184 | LI:235255.8:2000SEP08 | 1968849H1 | 771 | 969 |
| 184 | LI:235255.8:2000SEP08 | 630569H1 | 770 | 899 |
| 184 | LI:235255.8:2000SEP08 | 3051523H1 | 775 | 1066 |
| 184 | LI:235255.8:2000SEP08 | 6621315H1 | 796 | 1284 |
| 184 | LI:235255.8:2000SEP08 | 2791215H1 | 799 | 1103 |
| 184 | LI:235255.8:2000SEP08 | 1804236H1 | 803 | 1053 |
| 184 | LI:235255.8:2000SEP08 | 1460331H1 | 805 | 1044 |
| 184 | LI:235255.8:2000SEP08 | 70571366V1 | 813 | 1318 |
| 184 | LI:235255.8:2000SEP08 | 4835570H1 | 825 | 1092 |
| 184 | LI:235255.8:2000SEP08 | 3150504H1 | 837 | 951 |
| 184 | LI:235255.8:2000SEP08 | 7931885H1 | 839 | 1427 |
| 184 | LI:235255.8:2000SEP08 | 5041870H1 | 841 | 1081 |
| 184 | LI:235255.8:2000SEP08 | 2433958R6 | 849 | 1394 |
| 184 | LI:235255.8:2000SEP08 | 6840250H1 | 866 | 1015 |
| 184 | LI:235255.8:2000SEP08 | 4899142H2 | 863 | 1149 |
| 184 | LI:235255.8:2000SEP08 | 4898538H1 | 863 | 1138 |
| 184 | LI:235255.8:2000SEP08 | 7071147H1 | 876 | 1275 |
| 184 | LI:235255.8:2000SEP08 | 3663348H1 | 877 | 1153 |
| 184 | LI:235255.8:2000SEP08 | 5605778H1 | 878 | 992 |
| 184 | LI:235255.8:2000SEP08 | 7215091H1 | 878 | 1135 |
| 184 | LI:235255.8:2000SEP08 | 893148H1 | 906 | 1188 |
| 184 | LI:235255.8:2000SEP08 | 1652130H1 | 906 | 1145 |
| 184 | LI:235255.8:2000SEP08 | 2691823H1 | 923 | 1162 |
| 184 | LI:235255.8:2000SEP08 | 600445H1 | 927 | 1159 |
| 184 | LI:235255.8:2000SEP08 | 7658901J1 | 954 | 1477 |
| 184 | LI:235255.8:2000SEP08 | 3725892H1 | 974 | 1263 |
| 184 | LI:235255.8:2000SEP08 | 2272178H1 | 976 | 1247 |
| 184 | LI:235255.8:2000SEP08 | 3725965H 1 | 975 | 1256 |
| 184 | LI:235255.8:2000SEP08 | 6910727J1 | 986 | 1098 |
| 184 | LI:235255.8:2000SEP08 | 4859915H1 | 1000 | 1239 |
| 184 | LI:235255.8:2000SEP08 | 6883819J1 | 1014 | 1509 |
| 184 | LI:235255.8:2000SEP08 | 1361213H1 | 1018 | 1250 |
| 184 | LI:235255.8:2000SEP08 | 1361092F1 | 1018 | 1350 |
| 184 | LI:235255.8:2000SEP08 | 1718984T6 | 1029 | 1514 |
| 184 | LI:235255.8:2000SEP08 | 1705634T6 | 1033 | 1514 |
| 184 | LI:235255.8:2000SEP08 | 2047749H1 | 1037 | 1208 |
| 184 | LI:235255.8:2000SEP08 | 2128803H1 | 1041 | 1211 |
| 184 | LI:235255.8:2000SEP08 | 5290142H1 | 1041 | 1286 |
| 184 | LI:235255.8:2000SEP08 | 1641905H1 | 1053 | 1268 |
| 184 | LI:235255.8:2000SEP08 | 3089073H1 | 1061 | 1341 |
| 184 | LI:235255.8:2000SEP08 | 2418969H1 | 1085 | 1319 |
| 184 | LI:235255.8:2000SEP08 | 6163740H1 | 1094 | 1474 |
| 184 | LI:235255.8:2000SEP08 | g2900015 | 1097 | 1559 |
| 184 | LI:235255.8:2000SEP08 | 5108013H1 | 1104 | 1308 |
| 184 | LI:235255.8:2000SEP08 | 2433958T6 | 1107 | 1547 |
| 184 | LI:235255.8:2000SEP08 | 2100492H1 | 1112 | 1301 |
| 184 | LI:235255.8:2000SEP08 | g4896364 | 1132 | 1477 |
| 184 | LI:235255.8:2000SEP08 | g6044400 | 1149 | 1477 |
| 184 | LI:235255.8:2000SEP08 | 1655388T6 | 1149 | 1509 |
| 184 | LI:235255.8:2000SEP08 | 1439226H1 | 1161 | 1420 |
| 184 | LI:235255.8:2000SEP08 | 2239189H1 | 1162 | 1408 |
| 184 | LI:235255.8:2000SEP08 | g3229346 | 1173 | 1561 |
| 184 | LI:235255.8:2000SEP08 | 2071873T6 | 1203 | 1517 |
| 184 | LI:235255.8:2000SEP08 | 2070989H1 | 1205 | 1459 |
| 184 | LI:235255.8:2000SEP08 | 2096546H1 | 1222 | 1460 |
| 184 | LI:235255.8:2000SEP08 | g2873693 | 1236 | 1551 |
| 184 | LI:235255.8:2000SEP08 | 531 1408H1 | 1275 | 1477 |
| 184 | LI:235255.8:2000SEP08 | 1694442H1 | 1346 | 1560 |
| 184 | LI:235255.8:2000SEP08 | 3349083H1 | 1382 | 1556 |
| 185 | LI:237693.5:2000SEP08 | 5083439F8 | 1 | 528 |
| 185 | LI:237693.5:2000SEP08 | 2989661H1 | 1 | 301 |
| 185 | LI:237693.5:2000SEP08 | 2752263R6 | 2 | 478 |
| 185 | LI:237693.5:2000SEP08 | 5446248H1 | 1 | 264 |
| 185 | LI:237693.5:2000SEP08 | 2752263H1 | 2 | 269 |
| 185 | LI:237693.5:2000SEP08 | 2922381H1 | 24 | 287 |
| 185 | LI:237693.5:2000SEP08 | 2473969H1 | 33 | 243 |
| 185 | LI:237693.5:2000SEP08 | 2473969F6 | 33 | 243 |
| 185 | LI:237693.5:2000SEP08 | 3316534H1 | 54 | 296 |
| 185 | LI:237693.5:2000SEP08 | g4703345 | 65 | 509 |
| 185 | LI:237693.5:2000SEP08 | 3502790H1 | 312 | 587 |
| 186 | LI:433670.3:2000SEP08 | 3319844H1 | 1 | 268 |
| 186 | LI:433670.3:2000SEP08 | 3319844F6 | 1 | 302 |
| 186 | LI:433670.3:2000SEP08 | 7232871H1 | 179 | 787 |
| 186 | LI:433670.3:2000SEP08 | 4172439T6 | 662 | 1103 |
| 187 | LI:202943.4:2000SEP08 | 60210577LI1 | 1 | 432 |
| 187 | LI:202943.4:2000SEP08 | 70443926D1 | 31 | 494 |
| 187 | LI:202943.4:2000SEP08 | 70444091 D1 | 31 | 398 |
| 187 | LI:202943.4:2000SEP08 | 70443917D1 | 31 | 419 |
| 187 | LI:202943.4:2000SEP08 | 70442607D1 | 79 | 470 |
| 187 | LI:202943.4:2000SEP08 | 70444223D1 | 246 | 854 |
| 187 | LI:202943.4:2000SEP08 | 70444717D1 | 512 | 881 |
| 187 | LI:202943.4:2000SEP08 | 8108821H1 | 568 | 1216 |
| 187 | LI:202943.4:2000SEP08 | 70444084D1 | 629 | 1023 |
| 187 | LI:202943.4:2000SEP08 | 70442853D1 | 685 | 1115 |
| 187 | LI:202943.4:2000SEP08 | g2020942 | 809 | 1047 |
| 187 | LI:202943.4:2000SEP08 | 70443670D1 | 844 | 1182 |
| 187 | LI:202943.4:2000SEP08 | 8108821J1 | 943 | 1553 |
| 187 | LI:202943.4:2000SEP08 | 7152636H1 | 1063 | 1150 |
| 187 | LI:202943.4:2000SEP08 | 5092608H1 | 1106 | 1379 |
| 187 | LI:202943.4:2000SEP08 | 5089408H1 | 1106 | 1375 |
| 187 | LI:202943.4:2000SEP08 | 5091008H1 | 1106 | 1374 |
| 187 | LI:202943.4:2000SEP08 | 7151349H1 | 1163 | 1666 |
| 187 | LI:202943.4:2000SEP08 | 5639884H1 | 1241 | 1470 |
| 187 | LI:202943.4:2000SEP08 | 1226590R6 | 1279 | 1688 |
| 187 | LI:202943.4:2000SEP08 | 1226590H1 | 1279 | 1523 |
| 187 | LI:202943.4:2000SEP08 | g2020858 | 1285 | 1525 |
| 187 | LI:202943.4:2000SEP08 | 7213845H1 | 1347 | 1897 |
| 187 | LI:202943.4:2000SEP08 | 1226590T6 | 1488 | 1993 |
| 187 | LI:202943.4:2000SEP08 | 5092132H1 | 1620 | 1868 |
| 187 | LI:202943.4:2000SEP08 | 6469878H1 | 1729 | 2290 |
| 187 | LI:202943.4:2000SEP08 | 7594783H1 | 1923 | 2331 |
| 187 | LI:202943.4:2000SEP08 | 8113737H1 | 1946 | 2575 |
| 187 | LI:202943.4:2000SEP08 | 6472564H1 | 2047 | 2462 |
| 187 | LI:202943.4:2000SEP08 | 2579002H2 | 2080 | 2282 |
| 188 | LI:068682.1:2000SEP08 | g3307490 | 141 | 343 |
| 188 | LI:068682.1:2000SEP08 | 6829315H1 | 313 | 883 |
| 188 | LI:068682.1:2000SEP08 | g3109791 | 491 | 810 |
| 188 | LI:068682.1:2000SEP08 | g5452473 | 491 | 649 |
| 188 | LI:068682.1:2000SEP08 | g4372490 | 78 | 422 |
| 188 | LI:068682.1:2000SEP08 | g3805312 | 34 | 422 |
| 188 | LI:068682.1:2000SEP08 | g6043518 | 78 | 422 |
| 188 | LI:068682.1:2000SEP08 | g4564783 | 26 | 422 |
| 188 | LI:068682.1:2000SEP08 | g5438746 | 1 | 422 |
| 188 | LI:068682.1:2000SEP08 | g2954208 | 76 | 422 |
| 188 | LI:068682.1:2000SEP08 | g2954218 | 141 | 421 |
| 188 | LI:068682.1:2000SEP08 | g6838215 | 123 | 382 |
| 188 | LI:068682.1:2000SEP08 | 2011384H1 | 189 | 262 |
| 189 | LI:203301.3:2000SEP08 | 2613134T6 | 2172 | 2550 |
| 189 | LI:203301.3:2000SEP08 | 3904374H1 | 2189 | 2332 |
| 189 | LI:203301.3:2000SEP08 | g5810033 | 2192 | 2612 |
| 189 | LI:203301.3:2000SEP08 7931883H1 | 2214 | 2633 | |
| 189 | LI:203301.3:2000SEP08 | g5540842 | 2229 | 2602 |
| 189 | LI:203301.3:2000SEP08 | g7040110 | 2231 | 2602 |
| 189 | LI:203301.3:2000SEP08 | g6658405 | 2231 | 2608 |
| 189 | LI:203301.3:2000SEP08 | 7667756H1 | 1 | 611 |
| 189 | LI:203301.3:2000SEP08 | 7263722H1 | 55 | 606 |
| 189 | LI:203301.3:2000SEP08 | 2516560F6 | 58 | 548 |
| 189 | LI:203301.3:2000SEP08 | 2516560H1 | 58 | 402 |
| 189 | LI:203301.3:2000SEP08 | 5541375H1 | 57 | 257 |
| 189 | LI:203301.3:2000SEP08 | 60212962LI1 | 61 | 615 |
| 189 | LI:203301.3:2000SEP08 | 7283792H1 | 69 | 696 |
| 189 | LI:203301.3:2000SEP08 | 60212961LI1 | 69 | 545 |
| 189 | LI:203301.3:2000SEP08 | 267136H1 | 69 | 494 |
| 189 | LI:203301.3:2000SEP08 | 5182929H1 | 74 | 301 |
| 189 | LI:203301.3:2000SEP08 | 7159563H1 | 86 | 568 |
| 189 | LI:203301.3:2000SEP08 | 1234347F1 | 123 | 720 |
| 189 | LI:203301.3:2000SEP08 | 1234347F6 | 123 | 628 |
| 189 | LI:203301.3:2000SEP08 | 1234347H1 | 123 | 389 |
| 189 | LI:203301.3:2000SEP08 | g2079759 | 135 | 549 |
| 189 | LI:203301.3:2000SEP08 | g318174 | 146 | 498 |
| 189 | LI:203301.3:20003EP08 | 60212963LI1 | 234 | 677 |
| 189 | LI:203301.3:2000SEP08 | g1509838 | 280 | 523 |
| 189 | LI:203301.3:2000SEP08 | 2074934H1 | 299 | 560 |
| 189 | LI:203301.3:2000SEP08 | 2074934F6 | 299 | 551 |
| 189 | LI:203301.3:2000SEP08 | 2791 160F6 | 320 | 806 |
| 189 | LI:203301.3:2000SEP08 | 2791160H2 | 320 | 641 |
| 189 | LI:203301.3:2000SEP08 | 4310273H1 | 467 | 786 |
| 189 | LI:203301.3:2000SEP08 | 6609896H2 | 474 | 960 |
| 189 | LI:203301.3:2000SEP08 | g6474225 | 508 | 832 |
| 189 | LI:203301.3:2000SEP08 | 7087174H1 | 568 | 1086 |
| 189 | LI:203301.3:2000SEP08 | 1675180H1 | 723 | 931 |
| 189 | LI:203301.3:2000SEP08 | 7606685H1 | 717 | 1141 |
| 189 | LI:203301.3:2000SEP08 | 6308156H1 | 739 | 1116 |
| 189 | LI:203301.3:2000SEP08 | 3039416F6 | 956 | 1492 |
| 189 | LI:203301.3:2000SEP08 | 3039416H1 | 956 | 1223 |
| 189 | LI:203301.3:2000SEP08 | 7933647H1 | 1024 | 1706 |
| 189 | LI:203301.3:2000SEP08 | 7674856H2 | 1152 | 1709 |
| 189 | LI:203301.3:2000SEP08 | 3892582H1 | 1338 | 1621 |
| 189 | LI:203301.3:2000SEP08 | g618400 | 1359 | 1709 |
| 189 | LI:203301.3:2000SEP08 | 6531461H1 | 1579 | 2120 |
| 189 | LI:203301.3:2000SEP08 | 125847H1 | 1680 | 1862 |
| 189 | LI:203301.3:20003EP08 | 4769649H1 | 1770 | 2033 |
| 189 | LI:203301.3:2000SEP08 | 3405320H1 | 1771 | 2032 |
| 189 | LI:203301.3:2000SEP08 | 2791160T6 | 1930 | 2432 |
| 189 | LI:203301.3:2000SEP08 | 4080504H1 | 1937 | 2244 |
| 189 | LI:203301.3:2000SEP08 | 6609896T2 | 1941 | 2533 |
| 189 | LI:203301.3:2000SEP08 | 789847H1 | 1945 | 2178 |
| 189 | LI:203301.3:2000SEP08 | 790089H1 | 1946 | 2161 |
| 189 | LI:203301.3:2000SEP08 | 3039416T6 | 1962 | 2566 |
| 189 | LI:203301.3:2000SEP08 | 956099R7 | 1991 | 2394 |
| 189 | LI:203301.3:2000SEP08 | 956099H1 | 1991 | 2252 |
| 189 | LI:203301.3:2000SEP08 | g7237547 | 2035 | 2493 |
| 189 | LI:203301.3:2000SEP08 | g6464289 | 2062 | 2472 |
| 189 | LI:203301.3:2000SEP08 | 2074934T6 | 2078 | 2574 |
| 189 | LI:203301.3:2000SEP08 | g4310000 | 2084 | 2547 |
| 189 | LI:203301.3:2000SEP08 | 2187776T6 | 2086 | 2569 |
| 189 | LI:203301.3:2000SEP08 | 2187776F6 | 2093 | 2584 |
| 189 | LI:203301.3:2000SEP08 | 2187776H1 | 2093 | 2377 |
| 189 | LI:203301.3:2000SEP08 | 956099T6 | 2098 | 2568 |
| 189 | LI:203301.3:2000SEP08 | g4683266 | 2139 | 2608 |
| 189 | LI:203301.3:2000SEP08 | 7247184H1 | 2140 | 2722 |
| 189 | LI:203301.3:2000SEP08 | g3240807 | 2142 | 2612 |
| 189 | LI:203301.3:2000SEP08 | g3804586 | 2145 | 2605 |
| 189 | LI:203301.3:2000SEP08 | g5638660 | 2154 | 2602 |
| 189 | LI:203301.3:2000SEP08 | g4297425 | 2160 | 2602 |
| 189 | LI:203301.3:2000SEP08 | g4736851 | 2167 | 2602 |
| 189 | LI:203301.3:2000SEP08 | 2516560T6 | 2166 | 2550 |
| 189 | LI:203301.3:2000SEP08 | g7150807 | 2173 | 2610 |
| 189 | LI:203301.3:2000SEP08 | g3753686 | 2240 | 2617 |
| 189 | LI:203301.3:2000SEP08 | 66941 8H1 | 2286 | 2573 |
| 189 | LI:203301.3:2000SEP08 | 669124H1 | 2286 | 2558 |
| 189 | LI:203301.3:2000SEP08 | g5742262 | 2297 | 2602 |
| 189 | LI:203301.3:2000SEP08 | g1220014 | 2302 | 2615 |
| 189 | LI:203301.3:2000SEP08 | g4987993 | 2334 | 2602 |
| 189 | LI:203301.3:2000SEP08 | g3839358 | 2344 | 2602 |
| 189 | LI:203301.3:2000SEP08 | 4913428H1 | 2456 | 2741 |
| 189 | LI:203301.3:2000SEP08 | 3641081H1 | 2602 | 2728 |
| 189 | LI:203301.3:2000SEP08 | 3485460H1 | 2614 | 2890 |
| 189 | LI:203301.3:2000SEP08 | 4106769F6 | 2722 | 3162 |
| 189 | LI:203301.3:2000SEP08 | 4106769H1 | 2721 | 2910 |
| 190 | LI:020726.3:2000SEP08 | 70858739V1 | 458 | 1073 |
| 190 | LI:020726.3:2000SEP08 | 71225604V1 | 499 | 1009 |
| 190 | LI:020726.3:2000SEP08 | 70858001 V1 | 500 | 1062 |
| 190 | LI:020726.3:2000SEP08 | 70796452V1 | 508 | 901 |
| 190 | LI:020726.3:2000SEP08 | 71226010V1 | 563 | 1119 |
| 190 | LI:020726.3:2000SEP08 | 7179848H1 | 562 | 912 |
| 190 | LI:020726.3:2000SEP08 | 71224790V1 | 581 | 1124 |
| 190 | LI:020726.3:2000SEP08 | 70793655V1 | 639 | 743 |
| 190 | LI:020726.3:2000SEP08 | 70855458V1 | 701 | 1340 |
| 190 | LI:020726.3:2000SEP08 | 70858062V1 | 702 | 1306 |
| 190 | LI:020726.3:2000SEP08 | 71226289V1 | 729 | 1174 |
| 190 | LI:020726.3:2000SEP08 | 71224784V1 | 765 | 1232 |
| 190 | LI:020726.3:2000SEP08 | 71224903V1 | 774 | 1282 |
| 190 | LI:020726.3:2000SEP08 | 70861562V1 | 837 | 1436 |
| 190 | LI:020726.3:2000SEP08 | 70855548V1 | 835 | 1325 |
| 190 | LI:020726.3:2000SEP08 | 70855022V1 | 860 | 1390 |
| 190 | LI:020726.3:2000SEP08 | 7724233H1 | 854 | 1272 |
| 190 | LI:020726.3:2000SEP08 | 5311769H1 | 862 | 1049 |
| 190 | LI:020726.3:2000SEP08 | 5311769F8 | 862 | 1491 |
| 190 | LI:020726.3:2000SEP08 | 70857896V1 | 913 | 1567 |
| 190 | LI:020726.3:2000SEP08 | 70857337V1 | 915 | 1530 |
| 190 | LI:020726.3:2000SEP08 | 71225621V1 | 947 | 1531 |
| 190 | LI:020726.3:2000SEP08 | 71225381V1 | 981 | 1564 |
| 190 | LI:020726.3:2000SEP08 | 5990468H1 | 1046 | 1194 |
| 190 | LI:020726.3:2000SEP08 | 6059824F8 | 1061 | 1589 |
| 190 | LI:020726.3:2000SEP08 | 71225618V1 | 1091 | 1435 |
| 190 | LI:020726.3:2000SEP08 | 70858268V1 | 788 | 1411 |
| 190 | LI:020726.3:2000SEP08 | 70858492V1 | 790 | 1259 |
| 190 | LI:020726.3:2000SEP08 | 70796790V1 | 810 | 1065 |
| 190 | LI:020726.3:2000SEP08 | 71227063V1 | 812 | 1095 |
| 190 | LI:020726.3:2000SEP08 | 70858313V1 | 815 | 1364 |
| 190 | LI:020726.3:2000SEP08 | 8097890H1 | 1 | 501 |
| 190 | LI:020726.3:2000SEP08 | 7441541H1 | 13 | 484 |
| 190 | LI:020726.3:2000SEP08 | 70855180V1 | 777 | 1400 |
| 190 | LI:020726.3:2000SEP08 | 70854812V1 | 781 | 1360 |
| 190 | LI:020726.3:2000SEP08 | 71225057V1 | 314 | 873 |
| 190 | LI:020726.3:2000SEP08 | 70856238V1 | 1167 | 1506 |
| 190 | LI:020726.3:2000SEP08 | 7724233J1 | 1178 | 1737 |
| 190 | LI:020726.3:2000SEP08 | 70858568V1 | 1248 | 1728 |
| 190 | LI:020726.3:2000SEP08 | 70856388V1 | 1272 | 1750 |
| 190 | LI:020726.3:2000SEP08 | 2738605T6 | 1334 | 1877 |
| 190 | LI:020726.3:2000SEP08 | 71224942V1 | 1370 | 1816 |
| 190 | LI:020726.3:2000SEP08 | 71224964V1 | 1376 | 1874 |
| 190 | LI:020726.3:2000SEP08 | 273860117 | 1383 | 1810 |
| 190 | LI:020726.3:2000SEP08 | 5906145H1 | 1506 | 1811 |
| 190 | LI:020726.3:2000SEP08 | 5906145F6 | 1506 | 1790 |
| 190 | LI:020726.3:2000SEP08 | g1383637 | 1553 | 1914 |
| 190 | LI:020726.3:2000SEP08 | 7427246H1 | 1717 | 2139 |
| 191 | LI:027209.1:2000SEP08 | 1388139H1 | 1 | 262 |
| 191 | LI:027209.1:2000SEP08 | 2737908H1 | 3 | 238 |
| 191 | LI:027209.1:2000SEP08 | 2737908F6 | 3 | 395 |
| 191 | LI:027209.1:2000SEP08 | 6306041F8 | 5 | 651 |
| 191 | LI:027209.1:2000SEP08 | 6306041H1 | 5 | 464 |
| 191 | LI:027209.1:2000SEP08 | 70563274V1 | 229 | 974 |
| 191 | LI:027209.1:2000SEP08 | 70563246V1 | 340 | 843 |
| 191 | LI:027209.1:2000SEP08 | 70565819V1 | 440 | 1018 |
| 191 | LI:027209.1:2000SEP08 | 1904413H1 | 563 | 709 |
| 191 | LI:027209.1:2000SEP08 | 70563142V1 | 627 | 1120 |
| 191 | LI:027209.1:2000SEP08 | 70564681V1 | 633 | 1098 |
| 191 | LI:027209.1:2000SEP08 | 70565477V1 | 675 | 1279 |
| 191 | LI:027209.1:2000SEP08 | 70562989V1 | 740 | 1342 |
| 191 | LI:027209.1:2000SEP08 | 70563804V1 | 745 | 1341 |
| 191 | LI:027209.1:2000SEP08 | 70565738V1 | 758 | 1337 |
| 191 | LI:027209.1:2000SEP08 | 2739431H1 | 801 | 1034 |
| 191 | LI:027209.1:2000SEP08 | 2739431F6 | 801 | 1135 |
| 191 | LI:027209.1:2000SEP08 | 70564623V1 | 974 | 1362 |
| 191 | LI:027209.1:2000SEP08 | 6306041T8 | 982 | 1512 |
| 191 | LI:027209.1:2000SEP08 | 2737908T6 | 1116 | 1572 |
| 191 | LI:027209.1:2000SEP08 | 1265467H1 | 1494 | 1829 |
| 191 | LI:027209.1:2000SEP08 | 2819460T6 | 1496 | 1585 |
| 191 | LI:027209.1:2000SEP08 | 2819460F6 | 1503 | 1615 |
| 191 | LI:027209.1:2000SEP08 | 2819460H1 | 1504 | 1587 |
| 192 | LI:108819.1:2000SEP08 | 71304273V1 | 726 | 1132 |
| 192 | LI:108819.1:2000SEP08 | 2992317H1 | 831 | 1113 |
| 192 | LI:108819.1:2000SEP08 | 71156868V1 | 448 | 1074 |
| 192 | LI:108819.1:2000SEP08 | 71304495V1 | 622 | 1043 |
| 192 | LI:108819.1:2000SEP08 | 71158547V1 | 306 | 966 |
| 192 | LI:108819.1:2000SEP08 | g4293857 | 687 | 962 |
| 192 | LI:108819.1:2000SEP08 | 71158569V1 | 322 | 838 |
| 192 | LI:108819.1:2000SEP08 | 71303686V1 | 222 | 812 |
| 192 | LI:108819.1:2000SEP08 | 3565182H1 | 507 | 797 |
| 192 | LI:108819.1:2000SEP08 | 71155803V1 | 100 | 706 |
| 192 | LI:108819.1:2000SEP08 | 7161666F8 | 49 | 666 |
| 192 | LI:108819.1:2000SEP08 | 7017571F8 | 56 | 671 |
| 192 | LI:108819.1:2000SEP08 | 71155656V1 | 15 | 589 |
| 192 | LI:108819.1:2000SEP08 | 71156169V1 | 31 | 584 |
| 192 | LI:108819.1:2000SEP08 | 7007819H1 | 27 | 572 |
| 192 | LI:108819.1:2000SEP08 | 71158484V1 | 16 | 545 |
| 192 | LI:108819.1:2000SEP08 | 71158011V1 | 1 | 546 |
| 192 | LI:108819.1:2000SEP08 | 71304131V1 | 16 | 524 |
| 192 | LI:108819.1:2000SEP08 | 7008530H1 | 34 | 478 |
| 192 | LI:108819.1:2000SEP08 | 7161666H1 | 49 | 450 |
| 192 | LI:108819.1:2000SEP08 | 71303751V1 | 16 | 466 |
| 192 | LI:108819.1:2000SEP08 | 5643301F6 | 16 | 427 |
| 192 | LI:108819.1:2000SEP08 | 3277305H1 | 143 | 408 |
| 192 | LI:108819.1:2000SEP08 | 71156777V1 | 708 | 1251 |
| 192 | LI:108819.1:2000SEP08 | 7161666R8 | 722 | 1184 |
| 192 | LI:108819.1:2000SEP08 | 71304394V1 | 649 | 1132 |
| 192 | LI:108819.1:2000SEP08 | 71156129V1 | 593 | 1132 |
| 192 | LI:108819.1:2000SEP08 | 71303435V1 | 695 | 1132 |
| 192 | LI:108819.1:2000SEP08 | 71156071V1 | 721 | 1132 |
| 192 | LI:108819.1:2000SEP08 | 71155696V1 | 613 | 1132 |
| 192 | LI:108819.1:2000SEP08 | 71155196V1 | 750 | 1132 |
| 192 | LI:108819.1:2000SEP08 | 71302821V1 | 701 | 1132 |
| 192 | LI:108819.1:2000SEP08 | 5643301R8 | 955 | 1131 |
| 192 | LI:108819.1:2000SEP08 | 5643301R6 | 838 | 1130 |
| 192 | LI:108819.1:2000SEP08 | 5643301H1 | 16 | 281 |
| 192 | LI:108819.1:2000SEP08 | 7017571H1 | 57 | 226 |
| 192 | LI:108819.1:2000SEP08 | 71188981V1 | 16 | 78 |
| 193 | LI:021759.1:2000SEP08 | 7739693J1 | 1 | 427 |
| 193 | LI:021759.1:2000SEP08 | 6099303T6 | 1 | 403 |
| 193 | LI:021759.1:2000SEP08 | 7368206H1 | 35 | 559 |
| 193 | LI:021759.1:2000SEP08 | 6099303F6 | 76 | 524 |
| 193 | LI:021759.1:2000SEP08 | g6639940 | 75 | 483 |
| 193 | LI:021759.1:2000SEP08 | 6099303H1 | 280 | 524 |
| 193 | LI:021759.1:2000SEP08 | 7277665H1 | 299 | 778 |
| 193 | LI:021759.1:2000SEP08 | 7114645H1 | 354 | 845 |
| 193 | LI:021759.1:2000SEP08 | 7946017H2 | 352 | 972 |
| 193 | LI:021759.1:2000SEP08 | 2139551H1 | 402 | 648 |
| 193 | LI:021759.1:2000SEP08 | 6982815H1 | 523 | 1043 |
| 193 | LI:021759.1:2000SEP08 | 6982815F8 | 523 | 1098 |
| 193 | LI:021759.1:2000SEP08 | 6982815R8 | 527 | 1164 |
| 193 | LI:021759.1:2000SEP08 | 7379844H2 | 565 | 922 |
| 193 | LI:021759.1:2000SEP08 | 7454646H2 | 568 | 950 |
| 193 | LI:021759.1:2000SEP08 | 7098314H1 | 579 | 949 |
| 193 | LI:021759.1:2000SEP08 | 2966371F6 | 685 | 1166 |
| 193 | LI:021759.1:2000SEP08 | 2966371H1 | 686 | 989 |
| 193 | LI:021759.1:2000SEP08 | 7739693H1 | 745 | 1294 |
| 193 | LI:021759.1:2000SEP08 | g1642049 | 756 | 1057 |
| 193 | LI:021759.1:2000SEP08 | g1753778 | 957 | 1246 |
| 194 | LI:1165967.1:2000SEP08 | g6639152 | 1 | 415 |
| 194 | LI:1165967.1:2000SEP08 | 6795757H1 | 1 | 531 |
| 194 | LI:1165967.1:2000SEP08 | 6795757F8 | 1 523 | |
| 194 | LI:1165967.1:2000SEP08 | 6795757T8 | 1 | 425 |
| 194 | LI:1165967.1:2000SEP08 | 6790411T8 | 140 | 415 |
| 194 | LI:1165967.1:2000SEP08 | 6790411F8 | 141 | 406 |
| 194 | LI:1165967.1:2000SEP08 | 6790411H1 | 280 | 526 |
| 194 | LI:1165967.1:2000SEP08 | 6795161T8 | 301 | 421 |
| 194 | LI:1165967.1:2000SEP08 | 6795161F8 | 301 | 522 |
| 194 | LI:1165967.1:2000SEP08 | 6795161H1 | 301 | 512 |
| 195 | LI:1166315.1:2000SEP08 | 2996394H1 | 1 | 264 |
| 195 | LI:1166315.1:2000SEP08 | 2919416H2 | 28 | 275 |
| 195 | LI:1166315.1:2000SEP08 | 2972940H1 | 241 | 530 |
| 195 | LI:1166315.1:2000SEP08 | 2972940F6 | 241 | 389 |
| 195 | LI:1166315.1:2000SEP08 | 2829325H1 | 487 | 756 |
| 195 | LI:1166315.1:2000SEP08 | 2950735H1 | 526 | 796 |
| 195 | LI:1166315.1:2000SEP08 | 71601665V1 | 619 | 1055 |
| 195 | LI:1166315.1:2000SEP08 | 2972940T6 | 717 | 895 |
| 196 | LI:204626.1:2000SEP08 | 70901761V1 | 593 | 1048 |
| 196 | LI:204626.1:2000SEP08 | 1543453T6 | 610 | 1221 |
| 196 | LI:204626.1:2000SEP08 | 1921258H1 | 832 | 1046 |
| 196 | LI:204626.1:2000SEP08 | 1921258F6 | 833 | 1233 |
| 196 | LI:204626.1:2000SEP08 | 70902979V1 | 25 | 623 |
| 196 | LI:204626.1:2000SEP08 | 70902424V1 | 520 | 1141 |
| 196 | LI:204626.1:2000SEP08 | 4778273T9 | 559 | 1136 |
| 196 | LI:204626.1:2000SEP08 | 71269109V1 | 24 | 482 |
| 196 | LI:204626.1:2000SEP08 | 1543453R6 | 24 | 440 |
| 196 | LI:204626.1:2000SEP08 | 71268878V1 | 24 | 332 |
| 196 | LI:204626.1:2000SEP08 | 5055563F9 | 38 | 607 |
| 196 | LI:204626.1:2000SEP08 | 5055563H1 | 38 | 307 |
| 196 | LI:204626.1:2000SEP08 | 71269305V1 | 48 | 656 |
| 196 | LI:204626.1:2000SEP08 | 70899115V1 | 100 | 741 |
| 196 | LI:204626.1:2000SEP08 | 4778273F8 | 126 | 752 |
| 196 | LI:204626.1:2000SEP08 | 4778273H1 | 126 | 397 |
| 196 | LI:204626.1:2000SEP08 | 2724784H1 | 137 | 372 |
| 196 | LI:204626.1:2000SEP08 | 71269307V1 | 152 | 747 |
| 196 | LI:204626.1:2000SEP08 | 70901209V1 | 181 | 645 |
| 196 | LI:204626.1:2000SEP08 | 71269923V1 | 198 | 768 |
| 196 | LI:204626.1:2000SEP08 | 71270183V1 | 244 | 806 |
| 196 | LI:204626.1:2000SEP08 | 70900135V1 | 264 | 662 |
| 196 | LI:204626.1:2000SEP08 | 71270585V1 | 282 | 757 |
| 196 | LI:204626.1:2000SEP08 | 71268793V1 | 282 | 772 |
| 196 | LI:204626.1:2000SEP08 | 70899191V1 | 310 | 668 |
| 196 | LI:204626.1:2000SEP08 | 2925738H1 | 339 | 605 |
| 196 | LI:204626.1:2000SEP08 | 70901814V1 | 399 | 1015 |
| 196 | LI:204626.1:2000SEP08 | 71270451V1 | 484 | 1071 |
| 196 | LI:204626.1:2000SEP08 | 7931380H1 | 497 | 907 |
| 196 | LI:204626.1:2000SEP08 | 6868907H1 | 1 | 542 |
| 196 | LI:204626.1:2000SEP08 | 1543453H1 | 24 | 217 |
| 196 | LI:204626.1:2000SEP08 | 71269839V1 | 24 | 555 |
| 196 | LI:204626.1:2000SEP08 | 70902750V1 | 24 | 509 |
| 196 | LI:204626.1:2000SEP08 | 71269008V1 | 24 | 569 |
| 197 | LI:801140.1:2000SEP08 | 6103338H1 | 66 | 375 |
| 197 | LI:801140.1:2000SEP08 | 1415866T6 | 391 | 627 |
| 197 | LI:801140.1:2000SEP08 | 1445465H1 | 53 | 320 |
| 197 | LI:801140.1:2000SEP08 | 6103338F7 | 66 | 594 |
| 197 | LI:801140.1:2000SEP08 | g1442104 | 1 | 290 |
| 197 | LI:801140.1:2000SEP08 | g1439745 | 13 | 306 |
| 197 | LI:801140.1:2000SEP08 | 1445465T6 | 44 | 628 |
| 197 | LI:801140.1:2000SEP08 | 1445465F6 | 53 | 367 |
| 198 | LI:286639.1:2000SEP08 | g6569543 | 1 | 466 |
| 198 | LI:286639.1:2000SEP08 | 4138803H1 | 17 | 304 |
| 198 | LI:286639.1:2000SEP08 | 3595464H1 | 344 | 623 |
| 198 | LI:286639.1:2000SEP08 | 7687060J1 | 370 | 953 |
| 198 | LI:286639.1:2000SEP08 | 8031936J1 | 648 | 1260 |
| 198 | LI:286639.1:2000SEP08 | 5508394F6 | 681 | 1105 |
| 198 | LI:286639.1:2000SEP08 | 5508394H1 | 681 | 879 |
| 198 | LI:286639.1:2000SEP08 | g2025699 | 726 | 1057 |
| 198 | LI:286639.1:2000SEP08 | 3217093H1 | 740 | 1010 |
| 198 | LI:286639.1:2000SEP08 | 3217093F6 | 740 | 1172 |
| 198 | LI:286639.1:2000SEP08 | 7687060H1 | 1022 | 1613 |
| 198 | LI:286639.1:2000SEP08 | 4919304F6 | 1113 | 1524 |
| 198 | LI:286639.1:2000SEP08 | 5507629H1 | 1179 | 1448 |
| 198 | LI:286639.1:2000SEP08 | 5832442H1 | 1196 | 1449 |
| 198 | LI:286639.1:2000SEP08 | 70449421V1 | 1211 | 1798 |
| 198 | LI:286639.1:2000SEP08 | 301366R6 | 1211 | 1673 |
| 198 | LI:286639.1:2000SEP08 | 301366H1 | 1211 | 1427 |
| 198 | LI:286639.1:2000SEP08 | 301366T6 | 1243 | 1769 |
| 198 | LI:286639.1:2000SEP08 | 3217093T6 | 1310 | 1845 |
| 198 | LI:286639.1:2000SEP08 | 6178302H1 | 1388 | 1561 |
| 198 | LI:286639.1:2000SEP08 | 4919304H1 | 1393 | 1524 |
| 198 | LI:286639.1:2000SEP08 | 70450250V1 | 1444 | 1656 |
| 198 | LI:286639.1:2000SEP08 | g2057022 | 1461 | 1801 |
| 198 | LI:286639.1:2000SEP08 | g3933409 | 1496 | 1976 |
| 198 | LI:286639.1:2000SEP08 | g3446647 | 1520 | 1976 |
| 198 | LI:286639.1:2000SEP08 | g7037249 | 1645 | 2077 |
| 198 | LI:286639.1:2000SEP08 | 5508394R6 | 1791 | 2258 |
| 198 | LI:286639.1:2000SEP08 | g5812196 | 1868 | 1967 |
| 198 | LI:286639.1:2000SEP08 | g5397776 | 1882 | 2282 |
| 198 | LI:286639.1:2000SEP08 | g4739487 | 1905 | 2282 |
| 198 | LI:286639.1:2000SEP08 | g4739468 | 1907 | 2282 |
| 198 | LI:286639.1:2000SEP08 | 4249162F6 | 1966 | 2348 |
| 198 | LI:286639.1:2000SEP08 | 4249162H1 | 1966 | 2116 |
| 198 | LI:286639.1:2000SEP08 | g3109536 | 2092 | 2282 |
| 198 | LI:286639.1:2000SEP08 | g5446112 | 2093 | 2282 |
| 199 | LI:288905.4:2000SEP08 | 5514194R6 | 1047 | 1349 |
| 199 | LI:288905.4:2000SEP08 | 70956555V1 | 830 | 1349 |
| 199 | LI:288905.4:2000SEP08 | 70953630V1 | 810 | 1349 |
| 199 | LI:288905.4:2000SEP08 | 71532891 V1 | 1978 | 2337 |
| 199 | LI:288905.4:2000SEP08 | 71536825V1 | 1844 | 2331 |
| 199 | LI:288905.4:2000SEP08 | 71533046V1 | 1644 | 2320 |
| 199 | LI:288905.4:2000SEP08 | 2291521T6 | 1917 | 2293 |
| 199 | LI:288905.4:2000SEP08 | 71554987V1 | 1351 | 1986 |
| 199 | LI:288905.4:2000SEP08 | 71534196V1 | 1528 | 1972 |
| 199 | LI:288905.4:2000SEP08 | 71534802V1 | 1771 | 2336 |
| 199 | LI:288905.4:2000SEP08 | 5514194F6 | 740 | 1 150 |
| 199 | LI:288905.4:2000SEP08 | 7361951H1 | 609 | 1146 |
| 199 | LI:288905.4:2000SEP08 | 70955351V1 | 1003 | 1349 |
| 199 | LI:288905.4:2000SEP08 | 2291521H1 | 1351 | 1576 |
| 199 | LI:288905.4:2000SEP08 | 60217920D1 | 1132 | 1514 |
| 199 | LI:288905.4:2000SEP08 | 70953324V1 | 832 | 1349 |
| 199 | LI:288905.4:2000SEP08 | 70954278V1 | 994 | 1349 |
| 199 | LI:288905.4:2000SEP08 | 71525163V1 | 1351 | 1584 |
| 199 | LI:288905.4:2000SEP08 | 70956109V1 | 806 | 1349 |
| 199 | LI:288905.4:2000SEP08 | 60217921D1 | 1146 | 1318 |
| 199 | LI:288905.4:2000SEP08 | 71531757V1 | 1719 | 1827 |
| 199 | LI:288905.4:2000SEP08 | 2291521R6 | 1351 | 1797 |
| 199 | LI:288905.4:2000SEP08 | 6764762H1 | 1177 | 1786 |
| 199 | LI:288905.4:2000SEP08 | 60217917D1 | 1154 | 1628 |
| 199 | LI:288905.4:2000SEP08 | 7189423H2 | 1274 | 1837 |
| 199 | LI:288905.4:2000SEP08 | 5514194H1 | 740 | 958 |
| 199 | LI:288905.4:2000SEP08 | 6880661R8 | 201 | 822 |
| 199 | LI:288905.4:2000SEP08 | 6880661J1 | 190 | 822 |
| 199 | LI:288905.4:2000SEP08 | 8104285J1 | 1 | 564 |
| 199 | LI:288905.4:2000SEP08 | 8104285H1 | 12 | 564 |
| 199 | LI:288905.4:2000SEP08 | 71298950V1 | 944 | 1097 |
| 199 | LI:288905.4:2000SEP08 | 71284821V1 | 893 | 1203 |
| 199 | LI:288905.4:2000SEP08 | 71281371V1 | 925 | 1202 |
| 199 | LI:288905.4:2000SEP08 | 70955501 V1 | 893 | 1201 |
| 199 | LI:288905.4:2000SEP08 | 71533608V1 | 1351 | 1891 |
| 199 | LI:288905.4:2000SEP08 | 71532308V1 | 1351 | 1892 |
| 199 | LI:288905.4:2000SEP08 | 70954888V1 | 955 | 1349 |
| 199 | LI:288905.4:2000SEP08 | 71284717V1 | 883 | 1349 |
| 199 | LI:288905.4:2000SEP08 | 70955682V1 | 888 | 1340 |
| 199 | LI:288905.4:2000SEP08 | 70954068V1 | 875 | 1349 |
| 199 | LI:288905.4:2000SEP08 | 70955030V1 | 852 | 1349 |
| 199 | LI:288905.4:2000SEP08 | 71285344V1 | 928 | 1349 |
| 199 | LI:288905.4:2000SEP08 | 71285861 V1 | 740 | 1266 |
| 200 | LI:332161.1:2000SEP08 | 4695529H1 | 1091 | 1363 |
| 200 | LI:332161.1:2000SEP08 | 3243925H1 | 1044 | 1271 |
| 200 | LI:332161.1:2000SEP08 | 4712378H1 | 1127 | 1232 |
| 200 | LI:332161.1:2000SEP08 | 7947211H1 | 1379 | 1743 |
| 200 | LI:332161.1:2000SEP08 | 4031709H1 | 1435 | 1704 |
| 200 | LI:332161.1:2000SEP08 | g5424582 | 2587 | 3058 |
| 200 | LI:332161.1:2000SEP08 | 2757668H1 | 2497 | 2732 |
| 200 | LI:332161.1:2000SEP08 | 5208776H1 | 1792 | 2020 |
| 200 | LI:332161.1:2000SEP08 | 3962101H1 | 2445 | 2594 |
| 200 | LI:332161.1:2000SEP08 | 1401824T6 | 2449 | 3015 |
| 200 | LI:332161.1:20003EP08 | g4373231 | 2584 | 3063 |
| 200 | LI:332161.1:2000SEP08 | 2402168H1 | 1344 | 1579 |
| 200 | LI:332161.1:2000SEP08 | 5576650H1 | 2430 | 2691 |
| 200 | LI:332161.1:2000SEP08 | 3962694H1 | 2445 | 2758 |
| 200 | LI:332161.1:2000SEP08 | 2263264H1 | 2328 | 2567 |
| 200 | LI:332161.1:2000SEP08 | 6871139H1 | 2354 | 2893 |
| 200 | LI:332161.1:2000SEP08 | 1231884H1 | 2360 | 2592 |
| 200 | LI:332161.1:2000SEP08 | 6551431H1 | 2371 | 2924 |
| 200 | LI:332161.1:2000SEP08 | 4745464H1 | 2383 | 2665 |
| 200 | LI:332161.1:2000SEP08 | 2042175H1 | 2416 | 2696 |
| 200 | LI:332161.1:2000SEP08 | 2042159H1 | 2416 | 2694 |
| 200 | LI:332161.1:2000SEP08 | 6020347H1 | 2423 | 3045 |
| 200 | LI:332161.1:2000SEP08 | g3757300 | 2589 | 3047 |
| 200 | LI:332161.1:2000SEP08 | g832175 | 1753 | 2099 |
| 200 | LI:332161.1:2000SEP08 | 6390618H1 | 1794 | 2017 |
| 200 | LI:332161.1:2000SEP08 | 4421919H1 | 2533 | 2786 |
| 200 | LI:332161.1:2000SEP08 | 7357227H1 | 2569 | 2864 |
| 200 | LI:332161.1:2000SEP08 | 95673436 | 2568 | 3061 |
| 200 | LI:332161.1:2000SEP08 | 6851125H1 | 2525 | 3055 |
| 200 | LI:332161.1:2000SEP08 | 5850744H1 | 2530 | 2827 |
| 200 | LI:332161.1:2000SEP08 | 639590T6 | 2517 | 3024 |
| 200 | LI:332161.1:2000SEP08 | 750185H1 | 1997 | 2237 |
| 200 | LI:332161.1:2000SEP08 | g4076789 | 2693 | 3058 |
| 200 | LI:332161.1:2000SEP08 | 2151283H1 | 2513 | 2762 |
| 200 | LI:332161.1:2000SEP08 | 3144748H1 | 1973 | 2289 |
| 200 | LI:332161.1:2000SEP08 | g3873009 | 2714 | 3054 |
| 200 | LI:332161.1:2000SEP08 | g4763480 | 2719 | 3055 |
| 200 | LI:332161.1:2000SEP08 | g3694102 | 2729 | 3055 |
| 200 | LI:332161.1:2000SEP08 | 3145494R6 | 2758 | 3055 |
| 200 | LI:332161.1:2000SEP08 | 3145494T6 | 2758 | 3015 |
| 200 | LI:332161.1:2000SEP08 | 9883928 | 1855 | 2179 |
| 200 | LI:332161.1:2000SEP08 | g899746 | 1948 | 2239 |
| 200 | LI:332161.1:2000SEP08 | g1321230 | 1834 | 2239 |
| 200 | LI:332161.1:2000SEP08 | g4389793 | 1 | 455 |
| 200 | LI:332161.1:2000SEP08 | g2740716 | 1 | 455 |
| 200 | LI:332161.1:2000SEP08 | 7459406H1 | 488 | 949 |
| 200 | LI:332161.1:2000SEP08 | 663357R6 | 1656 | 1859 |
| 200 | LI:332161.1:2000SEP08 | 8113129H1 | 725 | 1172 |
| 200 | LI:332161.1:2000SEP08 | 639590H1 | 1155 | 1419 |
| 200 | LI:332161.1:2000SEP08 | 4842286H1 | 1568 | 1825 |
| 200 | LI:332161.1:2000SEP08 | 5846944H1 | 1588 | 1714 |
| 200 | LI:332161.1:2000SEP08 | g389596 | 1619 | 2012 |
| 200 | LI:332161.1:2000SEP08 | 663357H1 | 1656 | 1886 |
| 200 | LI:332161.1:2000SEP08 | 4030167H1 | 369 | 612 |
| 200 | LI:332161.1:2000SEP08 | 7191788H2 | 369 | 642 |
| 200 | LI:332161.1:2000SEP08 | 7583301H1 | 427 | 976 |
| 200 | LI:332161.1:2000SEP08 | 7947211J1 | 1367 | 1743 |
| 200 | LI:332161.1:2000SEP08 | 2285809H1 | 1678 | 1918 |
| 200 | LI:332161.1:2000SEP08 | 5919393H1 | 1749 | 1820 |
| 200 | LI:332161.1:2000SEP08 | 7354238H1 | 129 | 679 |
| 200 | LI:332161.1:2000SEP08 | 7946405J1 | 320 | 877 |
| 200 | LI:332161.1:2000SEP08 | g5450466 | 2578 | 3055 |
| 200 | LI:332161.1:2000SEP08 | g5544477 | 2579 | 3057 |
| 200 | LI:332161.1:2000SEP08 | 1286402H1 | 2091 | 2307 |
| 200 | LI:332161.1:2000SEP08 | 6124971H1 | 2094 | 2598 |
| 200 | LI:332161.1:2000SEP08 | 6748478H1 | 2101 | 2671 |
| 200 | LI:332161.1:2000SEP08 | 2084251H1 | 2115 | 2403 |
| 200 | LI:332161.1:2000SEP08 | 4853918H1 | 2154 | 2432 |
| 200 | LI:332161.1:2000SEP08 | 1401824F6 | 2154 | 2631 |
| 200 | LI:332161.1:2000SEP08 | 1398640H1 | 2154 | 2461 |
| 200 | LI:332161.1:2000SEP08 | 1401824H1 | 2154 | 2410 |
| 200 | LI:332161.1:2000SEP08 | 5158072H1 | 2196 | 2307 |
| 200 | LI:332161.1:2000SEP08 | 2108107H1 | 2228 | 2514 |
| 200 | LI:332161.1:2000SEP08 | 6558505H1 | 2240 | 2817 |
| 200 | LI:332161.1:2000SEP08 | 6558704H1 | 2240 | 2494 |
| 200 | LI:332161.1:2000SEP08 | g5446752 | 2600 | 3055 |
| 200 | LI:332161.1:2000SEP08 | g2242278 | 2632 | 3050 |
| 200 | LI:332161.1:2000SEP08 | g2158800 | 2636 | 3058 |
| 200 | LI:332161.1:2000SEP08 | 1296724H1 | 2644 | 2781 |
| 200 | LI:332161.1:2000SEP08 | 48561 19H1 | 2653 | 2946 |
| 200 | LI:332161.1:2000SEP08 | g1512955 | 2655 | 2788 |
| 200 | LI:332161.1:2000SEP08 | g1289773 | 2664 | 2961 |
| 200 | LI:332161.1:2000SEP08 | g519583 | 2683 | 3052 |
| 200 | LI:3321 61.1:2000SEP08 | g828934 | 2687 | 3055 |
| 200 | LI:332161.1:2000SEP08 | 3145494H1 | 2758 | 2935 |
| 200 | LI:332161.1:2000SEP08 | g519621 | 2765 | 3055 |
| 200 | LI:332161.1:2000SEP08 | g678058 | 2772 | 3055 |
| 200 | LI:332161.1:2000SEP08 | g3134420 | 2804 | 3052 |
| 200 | LI:332161.1:2000SEP08 | 2880485T6 | 2809 | 3014 |
| 200 | LI:332161.1:2000SEP08 | 989476H1 | 2824 | 3006 |
| 200 | LI:332161.1:2000SEP08 | 663357T6 | 2840 | 3005 |
| 200 | LI:332161.1:2000SEP08 | g519457 | 2888 | 3052 |
| 200 | LI:332161.1:2000SEP08 | g3933553 | 2937 | 3052 |
| 200 | LI:332161.1:2000SEP08 | g812751 | 2940 | 3048 |
| 200 | LI:332161.1:2000SEP08 | g2070684 | 2945 | 3025 |
| 200 | LI:332161.1:2000SEP08 | g832176 | 2987 | 3077 |
| 200 | LI:332161.1:2000SEP08 | 7065623H1 | 536 | 765 |
| 200 | LI:332161.1:2000SEP08 | 2879012H1 | 1334 | 1636 |
| 200 | LI:332161.1:2000SEP08 | 2880485H1 | 1334 | 1585 |
| 200 | LI:332161.1:2000SEP08 | 2880485F6 | 1333 | 1742 |
| 200 | LI:332161.1:2000SEP08 | 5599319H1 | 2497 | 2718 |
| 200 | LI:332161.1:2000SEP08 | 2151107F6 | 2513 | 2931 |
| 200 | LI:332161.1:2000SEP08 | 8114452H1 | 2089 | 2597 |
| 200 | LI:332161.1:2000SEP08 | 1286402F1 | 2091 | 2357 |
| 200 | LI:332161.1:2000SEP08 | 4783064H1 | 2089 | 2139 |
| 200 | LI:332161.1:2000SEP08 | g5740853 | 2575 | 3055 |
| 200 | LI:332161.1:2000SEP08 | g1162883 | 2013 | 2342 |
| 200 | LI:332161.1:2000SEP08 | 5719879H1 | 2039 | 2437 |
| 200 | LI:332161.1:2000SEP08 | 1597684F6 | 2050 | 2516 |
| 200 | LI:33216LI:2000SEP08 | 1597684H1 | 2050 | 2258 |
| 200 | LI:332161.1:2000SEP08 | 5290786H1 | 2052 | 2311 |
| 200 | LI:332161.1:2000SEP08 | 3243490H1 | 2006 | 2243 |
| 200 | LI:332161.1:2000SEP08 | g705533 | 2089 | 2251 |
| 200 | LI:332161.1:2000SEP08 | 2244532H1 | 2089 | 2207 |
| 200 | LI:332161.1:2000SEP08 | 6148317H1 | 2253 | 2752 |
| 200 | LI:332161.1:2000SEP08 | 7238430H1 | 2322 | 2657 |
| 200 | LI:332161.1:2000SEP08 | 6865396H1 | 2320 | 2873 |
| 200 | LI:332161.1:2000SEP08 | 6865595H1 | 2320 | 2847 |
| 200 | LI:332161.1:2000SEP08 | g2159107 | 2328 | 2715 |
| 200 | LI:332161.1:2000SEP08 | 2263256H1 | 2328 | 2567 |
| 200 | LI:332161.1:2000SEP08 | 6058081H1 | 2241 | 2307 |
| 200 | LI:332161.1:2000SEP08 | 1597684T6 | 2483 | 3012 |
| 200 | LI:332161.1:2000SEP08 | 4694344H1 | 2484 | 2590 |
| 200 | LI:332161.1:2000SEP08 | 4030167T8 | 2497 | 2963 |
| 201 | LI:184867.1:2000SEP08 | 2728616H1 | 39 | 290 |
| 201 | LI:184867.1:2000SEP08 | 70868482V1 | 288 | 920 |
| 201 | LI:184867.1:2000SEP08 | 4897718F6 | 342 | 738 |
| 201 | LI:184867.1:2000SEP08 | 70868801 V1 | 1342 | 1956 |
| 201 | LI:184867.1:2000SEP08 | 71229928V1 | 1560 | 2254 |
| 201 | LI:184867.1 :2000SEP08 | 70867474V1 | 920 | 1530 |
| 201 | LI:184867.1:2000SEP08 | 71230292V1 | 898 | 1427 |
| 201 | LI:184867.1:2000SEP08 | 71220802V1 | 1570 | 1837 |
| 201 | LI:184867.1:2000SEP08 | 70867220V1 | 1199 | 1738 |
| 201 | LI:184867.1:2000SEP08 | 2614428H1 | 1272 | 1523 |
| 201 | LI:184867.1:2000SEP08 | 70870340V1 | 1285 | 1456 |
| 201 | LI:184867.1:2000SEP08 | g3229291 | 1837 | 2254 |
| 201 | LI:184867.1:2000SEP08 | g4187447 | 1875 | 2257 |
| 201 | LI:184867.1:2000SEP08 | g3245634 | 1882 | 2251 |
| 201 | LI:184867.1:2000SEP08 | g3148017 | 1907 | 2248 |
| 201 | LI:184867.1:2000SEP08 | g661909 | 1925 | 2261 |
| 201 | LI:184867.1:2000SEP08 | g3238463 | 2003 | 2252 |
| 201 | LI:184867.1:2000SEP08 | 4897718T6 | 1725 | 2206 |
| 201 | LI:184867.1:2000SEP08 | g5630597 | 1816 | 2261 |
| 201 | LI:184867.1:2000SEP08 | 70838826V1 | 342 | 614 |
| 201 | LI:184867.1:2000SEP08 | 4897718H1 | 342 | 594 |
| 201 | LI:184867.1:2000SEP08 | 70868224V1 | 342 | 523 |
| 201 | LI:184867.1:2000SEP08 | 70870473V1 | 1292 | 1908 |
| 201 | LI:184867.1:2000SEP08 | 70870290V1 | 343 | 929 |
| 201 | LI:184867.1:2000SEP08 | 71229226V1 | 386 | 922 |
| 201 | LI:184867.1:2000SEP08 | 5876947H1 | 417 | 693 |
| 201 | LI:184867.1:2000SEP08 | 70870733V1 | 555 | 1027 |
| 201 | LI:184867.1:2000SEP08 | 70868874V1 | 622 | 814 |
| 201 | LI:184867.1:2000SEP08 | 70840206V1 | 662 | 1019 |
| 201 | LI:184867.1:2000SEP08 | 70869882V1 | 725 | 1390 |
| 201 | LI:184867.1:2000SEP08 | 70818463V1 | 785 | 967 |
| 201 | LI:184867.1:2000SEP08 | 70869040V1 | 783 | 1385 |
| 201 | LI:184867.1:2000SEP08 | 70867917V1 | 840 | 1007 |
| 201 | LI:184867.1:2000SEP08 | 7401989H1 | 1 | 582 |
| 201 | LI:184867.1:2000SEP08 | 70838881V1 | 974 | 1431 |
| 201 | LI:184867.1:2000SEP08 | 71220717V1 | 1157 | 1537 |
| 201 | LI:184867.1:2000SEP08 | 70868167V1 | 1118 | 1387 |
| 201 | LI:184867.1:2000SEP08 | 71229521V1 | 920 | 1445 |
| 201 | LI:184867.1:2000SEP08 | 71230330V1 | 963 | 1652 |
| 202 | LI:229932.4:2000SEP08 | 4203008H1 | 1875 | 1944 |
| 202 | LI:229932.4:2000SEP08 | g1664106 | 1875 | 1944 |
| 202 | LI:229932.4:2000SEP08 | 601 23139D2 | 1 | 306 |
| 202 | LI:229932.4:2000SEP08 | g993694 | 1035 | 1351 |
| 202 | LI:229932.4:2000SEP08 | g5513172 | 1046 | 1465 |
| 202 | LI:229932.4:2000SEP08 | 2866203H1 | 1038 | 1142 |
| 202 | LI:229932.4:2000SEP08 | 60123152D1 | 1 | 367 |
| 202 | LI:229932.4:2000SEP08 | 60123139D1 | 11 | 155 |
| 202 | LI:229932.4:2000SEP08 | g4898504 | 1080 | 1469 |
| 202 | LI:229932.4:2000SEP08 | g682774 | 1129 | 1410 |
| 202 | LI:229932.4:2000SEP08 | g3213293 | 1147 | 1611 |
| 202 | LI:229932.4:2000SEP08 | g3148379 | 1248 | 1465 |
| 202 | LI:229932.4:2000SEP08 | g2958900 | 1312 | 1431 |
| 202 | LI:229932.4:2000SEP08 | 6989224H1 | 72 | 606 |
| 202 | LI:229932.4:2000SEP08 | 6127857H1 | 134 | 609 |
| 202 | LI:229932.4:2000SEP08 | g4729416 | 1324 | 1465 |
| 202 | LI:229932.4:2000SEP08 | 8125520H1 | 1347 | 1699 |
| 202 | LI:229932.4:2000SEP08 | 5975549F8 | 1595 | 1944 |
| 202 | LI:229932.4:2000SEP08 | 5975549H1 | 1595 | 1944 |
| 202 | LI:229932.4:2000SEP08 | 7159562H1 | 1620 | 1944 |
| 202 | LI:229932.4:2000SEP08 | 4892481F8 | 1652 | 2227 |
| 202 | LI:229932.4:2000SEP08 | 4892481F9 | 1652 | 1759 |
| 202 | LI:229932.4:2000SEP08 | 4892481H1 | 1653 | 1755 |
| 202 | LI:229932.4:2000SEP08 | g7377738 | 1730 | 1944 |
| 202 | LI:229932.4:2000SEP08 | g706800 | 135 | 473 |
| 202 | LI:229932.4:2000SEP08 | g690740 | 145 | 519 |
| 202 | LI:229932.4:2000SEP08 | 4454268H1 | 511 | 782 |
| 202 | LI:229932.4:2000SEP08 | 7266188H1 | 589 | 1119 |
| 202 | LI:229932.4:2000SEP08 | 7932789H1 | 596 | 1209 |
| 202 | LI:229932.4:2000SEP08 | 3502996H1 | 702 | 989 |
| 202 | LI:229932.4:2000SEP08 | 6012315281 | 715 | 1385 |
| 202 | LI:229932.4:2000SEP08 | 1602161H1 | 797 | 992 |
| 202 | LI:229932.4:2000SEP08 | 6361212T8 | 827 | 1394 |
| 202 | LI:229932.4:2000SEP08 | 6217784H1 | 857 | 1357 |
| 202 | LI:229932.4:2000SEP08 | 6217935H1 | 859 | 1362 |
| 202 | LI:229932.4:2000SEP08 | 1473544R6 | 942 | 1392 |
| 202 | LI:229932.4:2000SEP08 | 1473544T1 | 942 | 1357 |
| 202 | LI:229932.4:2000SEP08 | 1473544H1 | 942 | 1195 |
| 202 | LI:229932.4:2000SEP08 | 6012313981 | 948 | 1368 |
| 202 | LI:229932.4:2000SEP08 | 6012485882 | 982 | 1368 |
| 202 | LI:229932.4:2000SEP08 | g4457589 | 994 | 1384 |
| 202 | LI:229932.4:2000SEP08 | 5945980H1 | 1846 | 1950 |
| 202 | LI:229932.4:2000SEP08 | g6033793 | 1010 | 1404 |
| 202 | LI:229932.4:2000SEP08 | 5763642T8 | 1019 | 1605 |
| 202 | LI:229932.4:2000SEP08 | g5905013 | 1017 | 1465 |
| 202 | LI:229932.4:2000SEP08 | 1473544T6 | 1026 | 1359 |
| 202 | LI:229932.4:2000SEP08 | g715576 | 1028 | 1415 |
| 202 | LI:229932.4:2000SEP08 | 167606H1 | 1 | 172 |
| 203 | LI:1189932.1:2000SEP08 | 638827T6 | 1597 | 2199 |
| 203 | LI:1189932.1:2000SEP08 | 71347095V1 | 1604 | 1888 |
| 203 | LI:1189932.1:2000SEP08 | 71346960V1 | 1290 | 1888 |
| 203 | LI:1189932.1:2000SEP08 | 6521632H1 | 1900 | 2034 |
| 203 | LI:1189932.1:2000SEP08 | g4984097 | 1919 | 2246 |
| 203 | LI:1189932.1:2000SEP08 | 713.43968V1 | 890 | 1460 |
| 203 | LI:1189932.1:2000SEP08 | 6434717H1 | 1144 | 1736 |
| 203 | LI:1189932.1:2000SEP08 | 179965R6 | 1175 | 1607 |
| 203 | LI:1189932.1:2000SEP08 | 6434786H1 | 1185 | 1736 |
| 203 | LI:1189932.1:2000SEP08 | 71345655V1 | 1276 | 1680 |
| 203 | LI:1189932.1:2000SEP08 | 71347202V1 | 789 | 1377 |
| 203 | LI:1189932.1:2000SEP08 | 71347021V1 | 791 | 1406 |
| 203 | LI:1189932.1:2000SEP08 | 71345916V1 | 819 | 1513 |
| 203 | LI:1189932.1:2000SEP08 | 71345414V1 | 151 | 752 |
| 203 | LI:1189932.1:2000SEP08 | 638659R6 | 151 | 699 |
| 203 | LI:1189932.1:2000SEP08 | 638827R6 | 151 | 632 |
| 203 | LI:1189932.1:2000SEP08 | 71344964V1 | 151 | 742 |
| 203 | LI:1189932.1:2000SEP08 | 71346724V1 | 846 | 1464 |
| 203 | LI:1189932.1:2000SEP08 | 7003793H1 | 856 | 1457 |
| 203 | LI:1189932.1:2000SEP08 | 7003893H1 | 883 | 1448 |
| 203 | LI:1189932.1:2000SEP08 | 4629950H1 | 2381 | 2442 |
| 203 | LI:1189932.1:2000SEP08 | 638827H1 | 151 | 420 |
| 203 | LI:1189932.1:2000SEP08 | 71345438V1 | 964 | 1612 |
| 203 | LI:1189932.1:2000SEP08 | g4851297 | 1811 | 2237 |
| 203 | LI:1189932.1:2000SEP08 | 71343050V1 | 151 | 717 |
| 203 | LI:1189932.1:2000SEP08 | 638659H1 | 151 | 423 |
| 203 | LI:1189932.1:2000SEP08 | g795000 | 1 326 | |
| 203 | LI:1189932.1:2000SEP08 | 60215345LI1 | 1 | 468 |
| 203 | LI:1189932.1:2000SEP08 | 6921792H1 | 20 639 | |
| 203 | LI:1189932.1:2000SEP08 | 6784528H2 | 20 | 498 |
| 203 | LI:1189932.1:2000SEP08 | 4605930H1 | 112 | 381 |
| 203 | LI:1189932.1:2000SEP08 | g1023546 | 131 | 419 |
| 203 | LI:1189932.1:2000SEP08 | 71343136V1 | 140 | 812 |
| 203 | LI:1189932.1:2000SEP08 | 71229364V1 | 2281 | 2854 |
| 203 | LI:1189932.1:2000SEP08 | 71343284V1 | 826 | 1437 |
| 203 | LI:1189932.1:2000SEP08 | 71356122V1 | 833 | 1070 |
| 203 | LI:1189932.1:2000SEP08 | 71345092V1 | 547 | 1319 |
| 203 | LI:1189932.1:2000SEP08 | 71343184V1 | 826 | 1437 |
| 203 | LI:1189932.1:2000SEP08 | 6350160H1 | 1580 | 1844 |
| 203 | LI:1189932.1:2000SEP08 | 71348225V1 | 1518 | 1749 |
| 203 | LI:1189932.1:2000SEP08 | 179965H1 | 1396 | 1607 |
| 203 | LI:1189932.1:2000SEP08 | 4163204T9 | 1557 | 2124 |
| 203 | LI:1189932.1:2000SEP08 | 2775552H1 | 2281 | 2439 |
| 203 | LI:1189932.1:2000SEP08 | 71345088V1 | 900 | 1535 |
| 203 | LI:1189932.1:2000SEP08 | 71348417V1986 | 1550 | |
| 203 | LI:1189932.1:2000SEP08 | 71343845V1 | 985 | 1258 |
| 203 | LI:1189932.1:2000SEP08 | 71347640V1 | 1093 | 1543 |
| 203 | LI:1189932.1:2000SEP08 | 6521732H1 | 1900 | 2034 |
| 203 | LI:1189932.1:2000SEP08 | 71345079V1 | 151 | 847 |
| 203 | LI:1189932.1:2000SEP08 | 638827R1 | 151 | 710 |
| 203 | LI:1189932.1:2000SEP08 | 71348114V1 | 150 | 663 |
| 203 | LI:1189932.1:2000SEP08 | 71344212V1 | 151 | 701 |
| 203 | LI:1189932.1:2000SEP08 | 70870383V1 | 2281 | 2794 |
| 203 | LI:1189932.1:2000SEP08 | 2775552F6 | 2281 | 2439 |
| 203 | LI:1189932.1:2000SEP08 | 71345071V1 | 152 | 264 |
| 203 | LI:1189932.1:2000SEP08 | 71342715V1 | 714 | 1486 |
| 203 | LI:1189932.1:2000SEP08 | 4163204F8 | 720 | 1141 |
| 203 | LI:1189932.1:2000SEP08 | 4163204H1 | 720 | 1032 |
| 203 | LI:1189932.1:2000SEP08 | 71355513V1 | 788 | 1116 |
| 203 | LI:1189932.1:2000SEP08 | 71348014V1 | 441 | 1099 |
| 203 | LI:1189932.1:2000SEP08 | 71345350V1 | 670 | 1274 |
| 203 | LI:1189932.1:2000SEP08 | 2121993F6 | 686 | 1159 |
| 203 | LI:1189932.1:2000SEP08 | 2121993H1 | 686 | 975 |
| 203 | LI:1189932.1:2000SEP08 | 3112883H1 | 714 | 999 |
| 203 | LI:1189932.1:2000SEP08 | 6358877H1 | 1337 | 1603 |
| 203 | LI:1189932.1:2000SEP08 | g2344131 | 1889 | 2238 |
| 203 | LI:1189932.1:2000SEP08 | 7222459H1 | 459 | 946 |
| 203 | LI:1189932.1:2000SEP08 | 71343691V1 | 1093 | 1543 |
| 203 | LI:1189932.1:2000SEP08 | 71343650V1 | 1093 | 1543 |
| 203 | LI:1189932.1:2000SEP08 | 71348384V1 | 577 | 1315 |
| 203 | LI:1189932.1:2000SEP08 | 6919315H1 | 301 | 713 |
| 203 | LI:1189932.1:2000SEP08 | 71344352V1 | 413 | 1178 |
| 203 | LI:1189932.1:2000SEP08 | 71345503V1 | 965 | 1401 |
| 203 | LI:1189932.1:2000SEP08 | 71345590V1 | 972 | 1550 |
| 203 | LI:1189932.1:2000SEP08 | 71342877V1 | 983 | 1625 |
| 203 | LI:1189932.1:2000SEP08 | 638659T6 | 1619 | 2198 |
| 203 | LI:1189932.1:2000SEP08 | 638827F1 | 1626 | 2237 |
| 203 | LI:1189932.1:2000SEP08 | 2121993T6 | 1709 | 2146 |
| 203 | LI:1189932.1:2000SEP08 | 2839037F6 | 1725 | 2161 |
| 203 | LI:1189932.1:2000SEP08 | 2839037H1 | 1725 | 1989 |
| 203 | LI:1189932.1:2000SEP08 | 5780748H1 | 1765 | 2041 |
| 203 | LI:1189932.1:2000SEP08 | 71342805V1 | 575 | 1178 |
| 203 | LI:1189932.1:2000SEP08 | 71347492V1 | 587 | 1255 |
| 203 | LI:1189932.1:2000SEP08 | 71342828V1 | 664 | 1287 |
| 203 | LI:1189932.1:2000SEP08 | 71345708V1 | 584 | 1309 |
| 203 | LI:1189932.1:2000SEP08 | 71347005V1 | 660 | 1290 |
| 203 | LI:1189932.1:2000SEP08 | 7933659H1 | 2161 | 2745 |
| 203 | LI:1189932.1:2000SEP08 | g4089780 | 2191 | 2246 |
| 203 | LI:1189932.1:2000SEP08 | 70867786V1 | 2281 | 2805 |
| 203 | LI:1189932.1:2000SEP08 | 71355035V1 | 413 | 864 |
| 203 | LI:1189932.1:2000SEP08 | 3461421H1 | 419 | 491 |
| 204 | LI:1076689.1:2000SEP08 | 6798226H1 | 1 | 510 |
| 204 | LI:1076689.1:2000SEP08 | 6797894H1 | 1 | 87 |
| 204 | LI:1076689.1:2000SEP08 | 6798226F8 | 40 | 428 |
| 204 | LI:1076689.1:2000SEP08 | 6797894F8 185 | 674 | |
| 204 | LI:1076689.1:2000SEP08 | 6795175H1 | 435 | 936 |
| 204 | LI:1076689.1:2000SEP08 | 67951 75F8 | 435 | 963 |
| 204 | LI:1076689.1:2000SEP08 | 67951 75T8 | 435 | 827 |
| 204 | LI:1076689.1:2000SEP08 | g1686420 | 526 | 928 |
| 205 | LI:415181.2:2000SEP08 | 5912551F6 | 1 | 282 |
| 205 | LI:415181.2:2000SEP08 | 5912551F8 | 2 | 599 |
| 205 | LI:415181.2:2000SEP08 | 71423279V1 | 347 | 926 |
| 205 | LI:415181.2:2000SEP08 | 5912551H1 | 1 | 277 |
| 205 | LI:415181.2:2000SEP08 | 71431480V1 | 404 | 1059 |
| 205 | LI:415181.2:2000SEP08 | 71426829V1 | 627 | 1210 |
| 205 | LI:415181.2:2000SEP08 | 71429277V1 | 885 | 1286 |
| 205 | LI:415181.2:2000SEP08 | 71430560V1 | 1220 | 1665 |
| 205 | LI:41518L2:2000SEP08 | 5912551T9 | 1272 | 1763 |
| 205 | LI:415181.2:2000SEP08 | 71428955V1 | 1387 | 1759 |
| 206 | LI:296358.1:2000SEP08 | 6255738H1 | 24 | 300 |
| 206 | LI:296358.1:2000SEP08 | 7279152H1 | 39 | 608 |
| 206 | LI:296358.1:2000SEP08 | 6422843H1 | 570 | 1050 |
| 206 | LI:296358.1:2000SEP08 | 7071023R8 | 673 | 1332 |
| 206 | LI:296358.1:2000SEP08 | 6422285H1 | 922 | 1091 |
| 206 | LI:296358.1:2000SEP08 | 6422285F8 | 922 | 1578 |
| 206 | LI:296358.1:2000SEP08 | 6255738T8 | 1107 | 1249 |
| 206 | LI:296358.1:2000SEP08 | 6258666T8 | 1108 | 1219 |
| 206 | LI:296358.1:2000SEP08 | 5545527H1 | 1228 | 1426 |
| 206 | LI:296358.1:2000SEP08 | 5618214R8 | 1376 | 1638 |
| 206 | LI:296358.1:2000SEP08 | 7071023F8 | 107 | 784 |
| 206 | LI:296358.1:2000SEP08 | 7071023H1 | 107 | 676 |
| 206 | LI:296358.1:2000SEP08 | 7316021H1 | 181 | 732 |
| 206 | LI:296358.1:2000SEP08 | 6258666F8 | 262 | 655 |
| 206 | LI:296358.1:2000SEP08 | 8116412H1 | 1 | 611 |
| 206 | LI:296358.1:2000SEP08 | g2008255 | 19 | 278 |
| 206 | LI:296358.1:2000SEP08 | 6255738F8 | 24 | 648 |
| 206 | LI:296358.1:2000SEP08 | 6258634H1 | 24 | 316 |
| 207 | LI:205186.3:2000SEP08 | 1689944H1 | 1 | 194 |
| 207 | LI:205186.3:2000SEP08 | 1322767H1 | 1 | 255 |
| 207 | LI:205186.3:2000SEP08 | 7352831H1 | 154 | 665 |
| 207 | LI:205186.3:2000SEP08 | 1545930H1 | 180 | 374 |
| 207 | LI:205186.3:2000SEP08 | 2271261H1 | 210 | 473 |
| 207 | LI:205186.3:2000SEP08 | 3499130H1 | 195 | 380 |
| 207 | LI:205186.3:2000SEP08 | 8065766J1 | 464 | 1029 |
| 207 | LI:205186.3:2000SEP08 | 6549558H1 | 747 | 1323 |
| 207 | LI:205186.3:2000SEP08 | 2750564R6 | 849 | 1388 |
| 207 | LI:205186.3:2000SEP08 | 2750564T6 | 849 | 1344 |
| 207 | LI:205186.3:2000SEP08 | 2750564H1 | 849 | 1124 |
| 207 | LI:205186.3:2000SEP08 | 4693201H1 | 849 | 1110 |
| 207 | LI:205186.3:2000SEP08 | g6989992 | 1055 | 1394 |
| 207 | LI:205186.3:2000SEP08 | 6893221J1 | 1085 | 1573 |
| 207 | LI:205186.3:2000SEP08 | 7358666H1 | 17 | 496 |
| 208 | LI:220537.2:2000SEP08 | 7228345H1 | 1 | 510 |
| 208 | LI:220537.2:2000SEP08 | 5390613F8 | 24 | 579 |
| 208 | LI:220537.2:2000SEP08 | 980265R6 | 327 | 786 |
| 208 | LI:220537.2:2000SEP08 | 70036886V1 | 1202 | 1670 |
| 208 | LI:220537.2:2000SEP08 | 70038905V1 | 1202 | 1532 |
| 208 | LI:220537.2:2000SEP08 | 3988928R6 | 1202 | 1693 |
| 208 | LI:220537.2:2000SEP08 | 70037558V1 | 1202 | 1625 |
| 208 | LI:220537.2:2000SEP08 | 70113012V1 | 1799 | 1927 |
| 208 | LI:220537.2:2000SEP08 | 70112530V1 | 1799 | 1886 |
| 208 | LI:220537.2:2000SEP08 | 4822576H1 | 1908 | 2170 |
| 208 | LI:220537.2:2000SEP08 | 70036263V1 | 1202 | 1550 |
| 208 | LI:220537.2:2000SEP08 | 70036856V1 | 1202 | 1559 |
| 208 | LI:220537.2:2000SEP08 | 3988928H1 | 1202 | 1391 |
| 208 | LI:220537.2:2000SEP08 | 4320977H1 | 1272 | 1562 |
| 208 | LI:220537.2:2000SEP08 | 70112984V1 | 1279 | 1395 |
| 208 | LI:220537.2:2000SEP08 | 70035465V1 | 1202 | 1607 |
| 208 | LI:220537.2:2000SEP08 | 4248779H1 | 120 | 271 |
| 208 | LI:220537.2:2000SEP08 | 700371 19V1 | 1432 | 2003 |
| 208 | LI:220537.2:2000SEP08 | 3988928T6 | 1450 | 1964 |
| 208 | LI:220537.2:2000SEP08 | 1681947H1 | 625 | 839 |
| 208 | LI:220537.2:2000SEP08 | 70036202V1 | 1474 | 1583 |
| 208 | LI:220537.2:2000SEP08 | 3436719H1 | 1334 | 1577 |
| 208 | LI:220537.2:2000SEP08 | 70039272V1 | 1374 | 1829 |
| 208 | LI:220537.2:2000SEP08 | 70037845V1 | 1389 | 1853 |
| 208 | LI:220537.2:2000SEP05 | 70035348V1 | 1376 | 1939 |
| 208 | LI:220537.2:2000SEP08 | 988064H1 | 327 | 607 |
| 208 | LI:220537.2:2000SEP08 | 7664878J1 | 387 | 1027 |
| 208 | LI:220537.2:2000SEP08 | 4248779R6 | 567 | 1023 |
| 208 | LI:220537.2:2000SEP08 | 4248779F6 | 120 | 545 |
| 208 | LI:220537.2:2000SEP08 | 7247233H 1 | 144 | 680 |
| 208 | LI:220537.2:2000SEP08 | 5390613H1 | 24 | 282 |
| 208 | LI:220537.2:2000SEP08 | g2022820 | 747 | 1095 |
| 208 | LI:220537.2:2000SEP08 | 7664878H1 | 951 | 1459 |
| 208 | LI:220537.2:2000SEP08 | g839595 | 1056 | 1292 |
| 208 | LI:220537.2:2000SEP08 | 70039014V1 | 1202 | 1800 |
| 208 | LI:220537.2:2000SEP08 | 70036954V1 | 1202 | 1664 |
| 208 | LI:220537.2:2000SEP08 | 70035507V1 | 1202 | 1625 |
| 209 | LI:248364.2:2000SEP08 | 814363R6 | 1500 | 1854 |
| 209 | LI:248364.2:2000SEP08 | 814363H1 | 1500 | 1729 |
| 209 | LI:248364.2:2000SEP08 | g3931760 | 1512 | 1907 |
| 209 | LI:248364.2:2000SEP08 | 5047370H1 | 1447 | 1737 |
| 209 | LI:248364.2:2000SEP08 | 814363R1 | 1500 | 1930 |
| 209 | LI:248364.2:2000SEP08 | 60209134LI1 | 1401 | 1704 |
| 209 | LI:248364.2:2000SEP08 | 60209135LI1 | 1408 | 1901 |
| 209 | LI:248364.2:2000SEP08 | 6758436H1 | 1100 | 1572 |
| 209 | LI:248364.2:2000SEP08 | 7754413H1 | 1124 | 1659 |
| 209 | LI:248364.2:2000SEP08 | 6608287H1 | 1244 | 1796 |
| 209 | LI:248364.2:2000SEP08 | 8053666J1 | 1323 | 1932 |
| 209 | LI:248364.2:2000SEP08 | 3071079H1 | 1364 | 1661 |
| 209 | LI:248364.2:2000SEP08 | 7608161J1 | 1374 | 1930 |
| 209 | LI:248364.2:2000SEP08 | 60201844V1 | 1245 | 1645 |
| 209 | LI:248364.2:2000SEP08 | g3873143 | 220 | 416 |
| 209 | LI:248364.2:2000SEP08 | 7468175H1456 | 898 | |
| 209 | LI:248364.2:2000SEP08 | 7363704H1 | 621 | 1110 |
| 209 | LI:248364.2:2000SEP08 | 7041822R8 | 693 | 1335 |
| 209 | LI:248364.2:2000SEP08 | 7608161H1 | 805 | 1211 |
| 209 | LI:248364.2:2000SEP08 | 7384566H1 | 843 | 1 160 |
| 209 | LI:248364.2:2000SEP08 | 7097046H1 | 1009 | 1496 |
| 209 | LI:248364.2:2000SEP08 | 7092325H1 | 146 | 384 |
| 209 | LI:248364.2:2000SEP08 | g3734235 | 180 | 494 |
| 209 | LI:248364.2:2000SEP08 | g6575611 | 182 | 600 |
| 209 | LI:248364.2:2000SEP08 | g1067627 | 187 | 516 |
| 209 | LI:248364.2:2000SEP08 | 6776971J1 | 1 | 596 |
| 209 | LI:248364.2:2000SEP08 | g7376252 | 137 | 623 |
| 209 | LI:248364.2:2000SEP08 | 6567819H1 | 146 | 688 |
| 210 | LI:2048338.1:2000SEP08 | 5786985H1 | 209 | 495 |
| 210 | LI:2048338.1:2000SEP08 | 5403369F8 | 19 | 594 |
| 210 | LI:2048338.1:2000SEP08 | 5403369T8 | 192 | 649 |
| 210 | LI:2048338.1:2000SEP08 | 5403369H1 | 1 | 213 |
| 211 | LI:1185203.8:2000SEP08 | 7388372H1 | 1 | 569 |
| 211 | LI:1185203.8:2000SEP08 | 6986073H1 | 207 | 560 |
| 211 | LI:1185203.8:2000SEP08 | 3242682H1 | 231 | 451 |
| 211 | LI:1185203.8:2000SEP08 | 2781642H1 | 443 | 560 |
| 212 | LI:021770.3:2000SEP08 | 4819519H1 | 138 | 408 |
| 212 | LI:021770.3:2000SEP08 | 7691849H1 | 187 | 844 |
| 212 | LI:021770.3:2000SEP08 | 041004H1 | 274 | 486 |
| 212 | LI:021770.3:2000SEP08 | 71047606V1 | 360 | 955 |
| 212 | LI:021770.3:2000SEP08 | 1684256H1 | 405 | 617 |
| 212 | LI:021770.3:2000SEP08 | 4970514H1 | 409 | 678 |
| 212 | LI:021770.3:2000SEP08 | 71045341V1 | 413 | 949 |
| 212 | LI:021770.3:2000SEP08 | 71048779V1 | 451 | 979 |
| 212 | LI:021770.3:2000SEP08 | 71048409V1 | 480 | 949 |
| 212 | LI:021770.3:2000SEP08 | 71046943V1 | 501 | 949 |
| 212 | LI:021770.3:2000SEP08 | 3778516H1 | 501 | 803 |
| 212 | LI:021770.3:2000SEP08 | 71045693V1 | 514 | 1213 |
| 212 | LI:021770.3:2000SEP08 | 7160062H1 | 521 | 1018 |
| 212 | LI:021770.3:2000SEP08 | 71048776V1 | 615 | 1251 |
| 212 | LI:021770.3:2000SEP08 | 71243858V1 | 616 | 949 |
| 212 | LI:021770.3:2000SEP08 | 4618506H1 | 658 | 895 |
| 212 | LI:021770.3:2000SEP08 | 71224692V1 | 659 | 925 |
| 212 | LI:021770.3:2000SEP08 | 71223211V1 | 666 | 922 |
| 212 | LI:021770.3:2000SEP08 | 5875430H1 | 665 | 934 |
| 212 | LI:021770.3:2000SEP08 | 71046758V1 | 703 | 1283 |
| 212 | LI:021770.3:2000SEP08 | 71244401V1 | 702 | 1199 |
| 212 | LI:021770.3:2000SEP08 | 71053755V1 | 864 | 949 |
| 212 | LI:021770.3:2000SEP08 | g6470601 | 1137 | 1291 |
| 212 | LI:021770.3:2000SEP08 | g2782960 | 1214 | 1350 |
| 212 | LI:021770.3:2000SEP08 | 6553139F8 | 1 | 544 |
| 212 | LI:021770.3:2000SEP08 | 2379209H1 | 3 | 225 |
| 212 | LI:021770.3:2000SEP08 | 5426218H1 | 33 | 152 |
| 212 | LI:021770.3:2000SEP08 | 7190209H2 | 47 | 600 |
| 212 | LI:021770.3:2000SEP08 | g706794 | 51 | 350 |
| 212 | LI:021770.3:2000SEP08 | g690668 | 53 | 394 |
| 212 | LI:021770.3:2000SEP08 | 6584640H1 | 74 | 364 |
| 212 | LI:021770.3:2000SEP08 | 5754744H1 | 67 | 565 |
| 212 | LI:021770.3:2000SEP08 | 7992554H1 | 74 | 655 |
| 212 | LI:021770.3:2000SEP08 | 5686262H1 | 111 | 367 |
| 212 | LI:021770.3:2000SEP08 | 5068302H1 | 111 | 383 |
| 212 | LI:021770.3:2000SEP08 | g1442458 | 121 | 333 |
| 212 | LI:021770.3:2000SEP08 | 5686262F6 | 125 | 660 |
| 212 | LI:021770.3:2000SEP08 | g2155825 | 134 | 628 |
| 212 | LI:021770.3:2000SEP08 | 4819519F7 | 137 | 720 |
| 212 | LI:021770.3:2000SEP08 | 71244184V1 | 137 | 681 |
| 212 | LI:021770.3:2000SEP08 | 71048027V1 | 138 | 717 |
| 213 | LI:1185841.1:2000SEP08 | 70797046V1 | 1572 | 2220 |
| 213 | LI:1185841.1:2000SEP08 | 71357714V1 | 1584 | 2164 |
| 213 | LI:1185841.1:2000SEP08 | 71358120V1 | 1569 | 2136 |
| 213 | LI:1185841.1:2000SEP08 | 71340047V1 | 105 | 480 |
| 213 | LI:1185841.1:2000SEP08 | 1531959H1 | 2306 | 2511 |
| 213 | LI:1185841.1:2000SEP08 | 71350132V1 | 181.8 | 2354 |
| 213 | LI:1185841.1:2000SEP08 | 71358382V1 | 1797 | 2375 |
| 213 | LI:1185841.1:2000SEP08 | 4491549H1 | 1794 | 2302 |
| 213 | LI:1185841.1:2000SEP08 | 70794172V1 | 1184 | 1661 |
| 213 | LI:1185841.1:2000SEP08 | g1859672 | 2890 | 3302 |
| 213 | LI:1185841.1:2000SEP08 | 557219R6 | 2901 | 3276 |
| 213 | LI:1185841.1:2000SEP08 | 557219H1 | 2901 | 3133 |
| 213 | LI:1185841.1:2000SEP08 | 2940096H2 | 2948 | 3195 |
| 213 | LI:1185841.1:2000SEP08 | 71359488V1 | 227 | 922 |
| 213 | LI:1185841.1:2000SEP08 | 70855819V1 | 1118 | 1288 |
| 213 | LI:1185841.1:2000SEP08 | 71356590V1 | 1125 | 1706 |
| 213 | LI:1185841.1:2000SEP08 | 70797154V1 | 1224 | 1792 |
| 213 | LI:1185841.1:2000SEP08 | 1340696H1 | 1238 | 1483 |
| 213 | LI:1185841.1:2000SEP08 | 1340976F6 | 1238 | 1593 |
| 213 | LI:1185841.1:2000SEP08 | 1340976H1 | 1238 | 1503 |
| 213 | LI:1185841.1:2000SEP08 | 70794785V1 | 1242 | 1556 |
| 213 | LI:1185841.1:2000SEP08 | 7726512J1 | 1256 | 1943 |
| 213 | LI:1185841.1:2000SEP08 | 3431125H1 | 1788 | 2051 |
| 213 | LI:1185841.1:2000SEP08 | 5079671H1 | 1429 | 1697 |
| 213 | LI:1185841.1:2000SEP08 | 71358505V1 | 1320 | 2038 |
| 213 | LI:1185841.1:2000SEP08 | g1939103 | 3421 | 3489 |
| 213 | LI:1185841.1:2000SEP08 | 410468H1 | 3051 | 3278 |
| 213 | LI:1185841.1:2000SEP08 | 768116H1 | 3066 | 3254 |
| 213 | LI:1185841.1:2000SEP08 | 1563060H1 | 2338 | 2562 |
| 213 | LI:1185841.1:2000SEP08 | 7701224H1 | 2344 | 2839 |
| 213 | LI:1185841.1:2000SEP08 | 403211H1 | 2405 | 2498 |
| 213 | LI:1185841.1:2000SEP08 | g6657086 | 3070 | 3492 |
| 213 | LI:1185841.1:2000SEP08 | g1891053 | 2850 | 3039 |
| 213 | LI:1185841.1:2000SEP08 | g2240351 | 2443 | 2587 |
| 213 | LI:1185841.1:2000SEP08 | 3620556H1 | 2467 | 2775 |
| 213 | LI:1185841.1:2000SEP08 | 7961057J1 | 2486 | 3102 |
| 213 | LI:1185841.1:2000SEP08 | 3525517H1 | 2509 | 2782 |
| 213 | LI:1185841.1:2000SEP08 | 7756966J1 | 1320 | 1917 |
| 213 | LI:1185841.1:2000SEP08 | 8120661H1 | 175 | 791 |
| 213 | LI:1185841.1:2000SEP08 | 70260763V1 | 200 | 883 |
| 213 | LI:1185841.1:2000SEP08 | 7423370T1 | 2885 | 3389 |
| 213 | LI:1185841.1:2000SEP08 | 6264728H1 | 2418 | 2548 |
| 213 | LI:1185841.1:2000SEP08 | 71356148V1 | 387 | 1028 |
| 213 | LI:1185841.1:2000SEP08 | 7701224J1 | 2592 | 3076 |
| 213 | LI:1185841.1:2000SEP08 | 4901488H1 | 2606 | 2874 |
| 213 | LI:1185841.1:2000SEP08 | 1735162H1 | 1910 | 2143 |
| 213 | LI:1185841.1:2000SEP08 | 5456184H1 | 1981 | 2203 |
| 213 | LI:1185841.1:2000SEP08 | 495726216 | 2009 | 2374 |
| 213 | LI:1185841.1:2000SEP08 | 71227380V1 | 2067 | 2185 |
| 213 | LI:1185841.1:2000SEP08 | 71357174V1 | 851 | 1433 |
| 213 | LI:1185841.1:2000SEP08 | 71360376V1 | 2128 | 2807 |
| 213 | LI:1185841.1:2000SEP08 | g3330806 | 3099 | 3489 |
| 213 | LI:1185841.1:2000SEP08 | g2875051 | 3112 | 3499 |
| 213 | LI:1185841.1:2000SEP08 | 6544161T8 | 2840 | 3408 |
| 213 | LI:1185841.1:2000SEP08 | 70260101V1 | 394 | 947 |
| 213 | LI:1185841.1:2000SEP08 | 71359142V1 | 358 | 876 |
| 213 | LI:1185841.1:2000SEP08 | 7720571H1 | 2891 | 3410 |
| 213 | LI:1185841.1:2000SEP08 | 70794292V1 | 1373 | 2030 |
| 213 | LI:1185841.1:2000SEP08 | 70796541V1 | 1374 | 2042 |
| 213 | LI:1185841.1:2000SEP08 | 7953629H1 | 1399 | 1892 |
| 213 | LI:1185841.1:2000SEP08 | 467549619 | 2883 | 3382 |
| 213 | LI:1185841.1:2000SEP08 | 71357522V1 | 2317 | 2945 |
| 213 | LI:1185841.1:2000SEP08 | 71356520V1 | 1886 | 2543 |
| 213 | LI:1185841.1:2000SEP08 | 71228048V1 | 1501 | 1722 |
| 213 | LI:1185841.1:2000SEP08 | 70818188V1 | 1508 | 2194 |
| 213 | LI:1185841.1:2000SEP08 | 71356831V1 | 1524 | 2186 |
| 213 | LI:1185841.1:2000SEP08 | 71360494V1 | 1524 | 2149 |
| 213 | LI:1185841.1:2000SEP08 | g4971028 | 3042 | 3495 |
| 213 | LI:1185841.1:2000SEP08 | 6264628H1 | 2425 | 2998 |
| 213 | LI:1185841.1:2000SEP08 | g4188980 | 3426 | 3489 |
| 213 | LI:1185841.1:2000SEP08 | 7756973J1 | 1299 | 1940 |
| 213 | LI:1185841.1:2000SEP08 | 726455R1 | 2224 | 2777 |
| 213 | LI:1185841.1:2000SEP08 | 726455H1 | 2224 | 2469 |
| 213 | LI:1185841.1:2000SEP08 | 2443111H1 | 2234 | 2481 |
| 213 | LI:1185841.1:2000SEP08 | 6825995H1 | 2247 | 2859 |
| 213 | LI:1185841.1:2000SEP08 | 70796432V1 | 1480 | 2101 |
| 213 | LI:1185841.1:2000SEP08 | 6221963LI1 | 1481 | 1619 |
| 213 | LI:1185841.1:2000SEP08 | 224108916 | 3372 | 3452 |
| 213 | LI:1185841.1:2000SEP08 | 7720571J1 | 1819 | 2301 |
| 213 | LI:1185841.1:2000SEP08 | g2899088 | 3127 | 3490 |
| 213 | LI:1185841.1:2000SEP08 | g3086920 | 3131 | 3492 |
| 213 | LI:1185841.1:2000SEP08 | 71356773V1 | 1781 | 2374 |
| 213 | LI:1185841.1:2000SEP08 | 71357337V1 | 891 | 1447 |
| 213 | LI:1185841.1:2000SEP08 | 4056381H1 | 895 | 1042 |
| 213 | LI:1185841.1:2000SEP08 | 2445025F6 | 1880 | 2065 |
| 213 | LI:1185841.1:2000SEP08 | 71358864V1 | 1322 | 2002 |
| 213 | LI:1185841.1:2000SEP08 | 71355972V1 | 1261 | 1874 |
| 213 | LI:1185841.1:2000SEP08 | 3542153H1 | 1892 | 2183 |
| 213 | LI:1185841.1:2000SEP08 | 71354984V1 | 1337 | 2068 |
| 213 | LI:1185841.1:2000SEP08 | 70263217V1 | 43 | 568 |
| 213 | LI:1185841.1:2000SEP08 | 70260615V1 | 43 | 517 |
| 213 | LI:1185841.1:2000SEP08 | 71356686V1 | 1892 | 2542 |
| 213 | LI:1185841.1:2000SEP08 | g1963392 | 2195 | 2563 |
| 213 | LI:1185841.1:2000SEP08 | 71228338V1 | 1138 | 1461 |
| 213 | LI:1185841.1:2000SEP08 | 70796159V1 | 860 | 1476 |
| 213 | LI:1185841.1:2000SEP08 | 70794910V1 | 1327 | 1922 |
| 213 | LI:1185841.1:2000SEP08 | 71358281V1 | 1748 | 2081 |
| 213 | LI:1185841.1:2000SEP08 | 522546H1 | 1751 | 2003 |
| 213 | LI:1185841.1:2000SEP08 | 4306643H2 | 1751 | 2033 |
| 213 | LI:1185841.1:2000SEP08 | 444203H1 | 2996 | 3235 |
| 213 | LI:1185841.1:2000SEP08 | g2583594 | 3138 | 3491 |
| 213 | LI:1185841.1:2000SEP08 | 71355056V1 | 280 | 935 |
| 213 | LI:1185841.1:2000SEP08 | 7756966H1 | 254 | 932 |
| 213 | LI:1185841.1:2000SEP08 | 71357821V1 | 1622 | 2404 |
| 213 | LI:1185841.1:2000SEP08 | 2432568H1 | 1421 | 1655 |
| 213 | LI:1185841.1:2000SEP08 | 3799411H1 | 1729 | 2037 |
| 213 | LI:1185841.1:2000SEP08 | 71354837V1 | 2085 | 2726 |
| 213 | LI:1185841.1:2000SEP08 | g4394665 | 3126 | 3492 |
| 213 | LI:1185841.1:2000SEP08 | 6455582H1 | 229 | 801 |
| 213 | LI:1185841.1:2000SEP08 | 71360211V1 | 1308 | 1959 |
| 213 | LI:1185841.1:2000SEP08 | g3428350 | 3034 | 3492 |
| 213 | LI:1185841.1:2000SEP08 | 2686855T6 | 3037 | 3451 |
| 213 | LI:1185841.1:2000SEP08 | 8041383H1 | 1840 | 2532 |
| 213 | LI:1185841.1:2000SEP08 | 71228091V1 | 1468 | 1715 |
| 213 | LI:1185841.1:2000SEP08 | 71356081V1 | 155 | 794 |
| 213 | LI:1185841.1:2000SEP08 | g2240350 | 3420 | 3488 |
| 213 | LI:1185841.1:2000SEP08 | 7312791H1 | 1 | 535 |
| 213 | LI:1185841.1:2000SEP08 | 3178743F6 | 43 | 627 |
| 213 | LI:1185841.1:2000SEP08 | 70261898V1 | 43 | 608 |
| 213 | LI:1185841.1:2000SEP08 | 70795477V1 | 1468 | 1884 |
| 213 | LI:1185841.1:2000SEP08 | 70863682V1 | 1467 | 1715 |
| 213 | LI:1185841.1:2000SEP08 | 71337137V1 | 1478 | 1991 |
| 213 | LI:1185841.1:2000SEP08 | 6221963LI2 | 1475 | 1919 |
| 213 | LI:1185841.1:2000SEP08 | 2193051H1 | 2192 | 2469 |
| 213 | LI:1185841.1:2000SEP08 | 71359982V1 | 568 | 1252 |
| 213 | LI:1185841.1:2000SEP08 | 71355792V1 | 2773 | 3134 |
| 213 | LI:1185841.1:2000SEP08 | 3486637H1 | 865 | 1149 |
| 213 | LI:1185841.1:2000SEP08 | 8010853H1 | 822 | 934 |
| 213 | LI:1185841.1:2000SEP08 | 3560004H1 | 829 | 1078 |
| 213 | LI:1185841.1:2000SEP08 | 71360318V1 | 848 | 1586 |
| 213 | LI:1185841.1:2000SEP08 | 70794615V1 | 1594 | 2080 |
| 213 | LI:1185841.1:2000SEP08 | 71357514V1 | 1633 | 2373 |
| 213 | LI:1185841.1:2000SEP08 | 71358947V1 | 920 | 1624 |
| 213 | LI:1185841.1:2000SEP08 | 4903918H2 | 592 | 886 |
| 213 | LI:1185841.1:2000SEP08 | 70796801V1 | 640 | 1342 |
| 213 | LI:1185841.1:2000SEP08 | 3000370H1 | 645 | 951 |
| 213 | LI:1185841.1:2000SEP08 | 6494524H1 | 2283 | 2826 |
| 213 | LI:1185841.1:2000SEP08 | 70793533V1 | 1165 | 1732 |
| 213 | LI:1185841.1:2000SEP08 | 1439270H1 | 2123 | 2370 |
| 213 | LI:1185841.1:2000SEP08 | 3000640H1 | 645 | 729 |
| 213 | LI:1185841.1:2000SEP08 | 71354806V1 | 1765 | 2416 |
| 213 | LI:1185841.1:2000SEP08 | 2445025H1 | 1880 | 2123 |
| 213 | LI:1185841.1:2000SEP08 | 71356157V1 | 1276 | 1836 |
| 213 | LI:1185841.1:2000SEP08 | 7726512H1 | 915 | 1586 |
| 213 | LI:1185841.1:2000SEP08 | 4532844H1 | 2887 | 2970 |
| 213 | LI:1185841.1:2000SEP08 | 71355177V1 | 1712 | 2510 |
| 213 | LI:1185841.1:2000SEP08 | 7756973H1 | 257 | 962 |
| 213 | LI:1185841.1:2000SEP08 | 6865917H1 | 2288 | 2857 |
| 213 | LI:1185841.1:2000SEP08 | 71355184V1 | 773 | 1399 |
| 213 | LI:1185841.1:2000SEP08 | 70796660V1 | 801 | 1500 |
| 213 | LI:1185841.1:2000SEP08 | g1011584 | 3176 | 3486 |
| 213 | LI:1185841.1:2000SEP08 | g4393181 | 3184 | 3490 |
| 213 | LI:1185841.1:2000SEP08 | 71357201V1 | 691 | 1327 |
| 213 | LI:1185841.1:2000SEP08 | 70864263V1 | 1501 | 1724 |
| 213 | LI:1185841.1:2000SEP08 | g3051168 | 3384 | 3486 |
| 213 | LI:1185841.1:2000SEP08 | 569396R6 | 105 | 607 |
| 213 | LI:1185841.1:2000SEP08 | 514078H1 | 3119 | 3333 |
| 213 | LI:1185841.1:2000SEP08 | 70860888V1 | 1053 | 1443 |
| 213 | LI:1185841.1:2000SEP08 | 71356532V1 | 1060 | 1760 |
| 213 | LI:1185841.1:2000SEP08 | 4265539H1 | 1744 | 2034 |
| 213 | LI:1185841.1:2000SEP08 | 70794539V1 | 821 | 1476 |
| 213 | LI:1185841.1:2000SEP08 | 70796837V1 | 1184 | 1912 |
| 213 | LI:1185841.1:2000SEP08 | 70818371V1 | 1221 | 1345 |
| 213 | LI:1185841.1:2000SEP08 | 71359841V1 | 284 | 963 |
| 213 | LI:1185841.1:2000SEP08 | 3178743H1 | 43 | 349 |
| 213 | LI:1185841.1:2000SEP08 | 70260353V1 | 54 | 641 |
| 213 | LI:1185841.1:2000SEP08 | 71358919V1 | 105 | 768 |
| 213 | LI:1185841.1:2000SEP08 | 71358990V1 | 105 | 752 |
| 213 | LI:1185841.1:2000SEP08 | 8118112H1 | 108 | 751 |
| 213 | LI:1185841.1:2000SEP08 | 70795792V1 | 1286 | 1942 |
| 213 | LI:1185841.1:2000SEP08 | 6182135H1 | 2549 | 2826 |
| 213 | LI:1185841.1:2000SEP08 | 71358084V1 | 711 | 1296 |
| 213 | LI:1185841.1:2000SEP08 | 6528429H1 | 752 | 899 |
| 213 | LI:1185841.1:2000SEP08 | 569396H1 | 105 | 397 |
| 213 | LI:1185841.1:2000SEP08 | 71358678V1 | 106 | 742 |
| 213 | LI:1185841.1:2000SEP08 | 7244833H1 | 115 | 542 |
| 213 | LI:1185841.1:2000SEP08 | 2831493H1 | 1490 | 1707 |
| 213 | LI:1185841.1:2000SEP08 | 2754923H1 | 2866 | 3137 |
| 213 | LI:1185841.1:2000SEP08 | g5631681 | 3141 | 3495 |
| 213 | LI:1185841.1:2000SEP08 | g1892259 | 3158 | 3488 |
| 213 | LI:1185841.1:2000SEP08 | 1879242H1 | 3176 | 3418 |
| 213 | LI:1185841.1:2000SEP08 | 2930052H1 | 3230 | 3486 |
| 213 | LI:1185841.1:2000SEP08 | 2445025T6 | 3286 | 3442 |
| 213 | LI:1185841.1:2000SEP08 | 1295163T6 | 3286 | 3452 |
| 213 | LI:1185841.1:2000SEP08 | g3834982 | 3337 | 3489 |
| 213 | LI:1185841.1:2000SEP08 | g3835256 | 3082 | 3495 |
| 213 | LI:1185841.1:2000SEP08 | 2394832H1 | 3096 | 3341 |
| 213 | LI:1185841.1:2000SEP08 | g2896588 | 2092 | 2484 |
| 213 | LI:1185841.1:2000SEP08 | 2467748H1 | 2100 | 2334 |
| 213 | LI:1185841.1:2000SEP08 | 71358284V1 | 2678 | 3220 |
| 213 | LI:1185841.1:2000SEP08 | 2241089F6 | 2743 | 3171 |
| 213 | LI:1185841.1:2000SEP08 | 3275630H1 | 2611 | 2893 |
| 213 | LI:1185841.1:2000SEP08 | 290499H1 | 2632 | 2978 |
| 213 | LI:1185841.1:2000SEP08 | 2241089H1 | 2743 | 2990 |
| 213 | LI:1185841.1:2000SEP08 | 2399690H1 | 2771 | 3008 |
| 213 | LI:1185841.1:2000SEP08 | 71356842V1 | 2140 | 2807 |
| 213 | LI:1185841.1:2000SEP08 | 71355693V1 | 2191 | 2945 |
| 213 | LI:1185841.1:2000SEP08 | 7961057H1 | 2184 | 2857 |
| 213 | LI:1185841.1:2000SEP08 | 71356810V1 | 458 | 1117 |
| 213 | LI:1185841.1:2000SEP08 | 71358113V1 | 469 | 1065 |
| 213 | LI:1185841.1:2000SEP08 | 8041383J1 | 501 | 1058 |
| 213 | LI:1185841.1:2000SEP08 | 1749048H1 | 515 | 783 |
| 213 | LI:1185841.1:2000SEP08 | 1749048F6 | 515 | 875 |
| 213 | LI:1185841.1:2000SEP08 | 70861180V1 | 515 | 727 |
| 213 | LI:1185841.1:2000SEP08 | 70794384V1 | 525 | 1062 |
| 213 | LI:1185841.1:2000SEP08 | 4957262F6 | 1524 | 1984 |
| 213 | LI:1185841.1:2000SEP08 | 4957262H1 | 1524 | 1809 |
| 213 | LI:1185841.1:2000SEP08 | 2443857F6 | 1524 | 1871 |
| 213 | LI:1185841.1:2000SEP08 | 2443857H1 | 1524 | 1772 |
| 213 | LI:1185841.1:2000SEP08 | 71336855V1 | 1564 | 1876 |
| 213 | LI:1185841.1:2000SEP08 | 70797142V1 | 1570 | 2252 |
| 213 | LI:1185841.1:2000SEP08 | 70794533V1 | 938 | 1489 |
| 213 | LI:1185841.1:2000SEP08 | 71357423V1 | 970 | 1608 |
| 213 | LI:1185841.1:2000SEP08 | 71351527V1 | 989 | 1192 |
| 213 | LI:1185841.1:2000SEP08 | 71351586V1 | 989 | 1192 |
| 213 | LI:1185841.1:2000SEP08 | 71347649V1 | 1032 | 1279 |
| 213 | LI:1185841.1:2000SEP08 | 3518889H1 | 1044 | 1368 |
| 214 | LI:1181710.1:2000SEP08 | 1476485H1 | 1 | 85 |
| 214 | LI:1181710.1:2000SEP08 | 1476477H1 | 1 | 205 |
| 214 | LI:1181710.1:2000SEP08 | 1476477H6 | 1 | 207 |
| 214 | LI:1181710.1:2000SEP08 | 1476477F6 | 1 | 546 |
| 214 | LI:1181710.1:2000SEP08 | 4398045H1 | 1 | 226 |
| 214 | LI:1181710.1:2000SEP08 | 105065H1 | 327 | 504 |
| 214 | LI:1181710.1:2000SEP08 | 1476477T6 | 122 | 543 |
| 214 | LI:1181710.1:2000SEP08 | 105065R1 | 327 | 461 |
| 215 | LI:2048959.1:2000SEP08 | g1809909 | 64 | 414 |
| 215 | LI:2048959.1:2000SEP08 | 332540217 | 1 | 458 |
| 215 | LI:2048959.1:2000SEP08 | g1757886 | 64 | 317 |
| 216 | LI:798494.1:2000SEP08 | 2110417H1 | 559 | 823 |
| 216 | LI:798494.1:2000SEP08 | 1398471H1 | 270 | 388 |
| 216 | LI:798494.1:2000SEP08 | g5880265 | 1 | 409 |
| 216 | LI:798494.1:2000SEP08 | 1398471F6 | 151 | 560 |
| 216 | LI:798494.1:2000SEP08 | 3382640F8 | 498 | 966 |
| 216 | LI:798494.1:2000SEP08 | 1399832H1 | 270 | 377 |
| 216 | LI:798494.1:2000SEP08 | g4690049 | 1 | 330 |
| 217 | LI:2049223.1:2000SEP08 | 4914013T8 | 166 | 672 |
| 217 | LI:2049223.1:2000SEP08 | g2907281 | 202 | 361 |
| 217 | LI:2049223.1:2000SEP08 | 4914013H1 | 1 | 255 |
| 218 | LI:1177833.1:2000SEP08 | 5502992F6 | 717 | 1037 |
| 218 | LI:1177833.1:2000SEP08 | 5878049F8 | 595 | 1070 |
| 218 | LI:1177833.1:2000SEP08 | 2327457R6 | 13 | 402 |
| 218 | LI:1177833.1:2000SEP08 | 4216837H1 | 739 | 996 |
| 218 | LI:1177833.1:2000SEP08 | 2205395H1 | 712 | 962 |
| 218 | LI:1177833.1:2000SEP08 | 2725166F6 | 620 | 1070 |
| 218 | LI:1177833.1:2000SEP08 | 2725166H1 | 620 | 780 |
| 218 | LI:1177833.1:2000SEP08 | 2327449T6 | 1 | 288 |
| 218 | LI:1177833.1:2000SEP08 | 75952LI6H1 | 502 | 945 |
| 218 | LI:1177833.1:2000SEP08 | 5878049H1 | 573 | 766 |
| 218 | LI:1177833.1:2000SEP08 | 2206408F6 | 712 | 1069 |
| 218 | LI:1177833.1:2000SEP08 | 4616581H1 | 823 | 1070 |
| 218 | LI:1177833.1:2000SEP08 | 2206408H1 | 712 | 966 |
| 218 | LI:1177833.1:2000SEP08 | g1691064 | 591 | 1034 |
| 218 | LI:1177833.1:2000SEP08 | 2327457T6 | 1 | 364 |
| 218 | LI:1177833.1:2000SEP08 | 2327449H1 | 4 | 248 |
| 218 | LI:1177833.1:2000SEP08 | 2327449R6 | 13 | 408 |
| 218 | LI:1177833.1:2000SEP08 | g1986375 | 288 | 629 |
| 218 | LI:1177833.1:2000SEP08 | 5693661H1 | 477 | 741 |
| 218 | LI:1177833.1:2000SEP08 | 5693661F8 | 482 | 1041 |
| 218 | LI:1177833.1:2000SEP08 | 5502992R6 | 623 | 1075 |
| 218 | LI:1177833.1:2000SEP08 | 2815519H1 | 877 | 1067 |
| 218 | LI:1177833.1:2000SEP08 | 2906529H1 | 804 | 1070 |
| 218 | LI:1177833.1:2000SEP08 | 5716504H1 | 657 | 945 |
| 219 | LI:2049267.1:2000SEP08 | 5809894H1 | 1 | 182 |
| 219 | LI:2049267.1:2000SEP08 | g6027870 | 11 | 309 |
| 219 | LI:2049267.1:2000SEP08 | g6132189 | 94 | 343 |
| 219 | LI:2049267.1:2000SEP08 | g1953935 | 110 | 360 |
| 220 | LI:1165939.1:2000SEP08 | 2870408F7 | 382 | 819 |
| 220 | LI:1165939.1:2000SEP08 | 91059584 | 419 | 690 |
| 220 | LI:1165939.1:2000SEP08 | 2870424H1 | 385 | 595 |
| 220 | LI:1165939.1:2000SEP08 | 2638526H1 | 249 | 368 |
| 220 | LI:1165939.1:2000SEP08 | g2180031 | 391 | 746 |
| 220 | LI:1165939.1:2000SEP08 | 2878289H1 | 382 | 662 |
| 220 | LI:1165939.1:2000SEP08 | 2870408H1 | 382 | 658 |
| 220 | LI:1165939.1:2000SEP08 | 2878289F6 | 382 | 887 |
| 220 | LI:1165939.1:2000SEP08 | 6176589F8 | 58 | 707 |
| 220 | LI:1165939.1:2000SEP08 | 8016005J1 | 1 | 660 |
| 220 | LI:1165939.1:2000SEP08 | 3201201T6 | 65 | 587 |
| 221 | LI:1170958.1:2000SEP08 | 6311083H1 | 1 | 558 |
| 221 | LI:1170958.1:2000SEP08 | 5543192T6 | 61 | 600 |
| 221 | LI:1170958.1:2000SEP08 | 3042046T6 | 91 | 601 |
| 221 | LI:1170958.1:2000SEP08 | g1261341 | 380 | 555 |
| 221 | LI:1170958.1:2000SEP08 | 5694986H1 | 504 | 643 |
| 221 | LI:1170958.1:2000SEP08 | 5694986F8 | 527 | 643 |
| 221 | LI:1170958.1:2000SEP08 | 6311083F8 | 1 | 377 |
| 222 | LI:1089827.1:2000SEP08 | 4826769H1 | 810 | 1064 |
| 222 | LI:1089827.1:2000SEP08 | 6025464H1 | 506 | 742 |
| 222 | LI:1089827.1:2000SEP08 | 753783H1 | 1100 | 1328 |
| 222 | LI:1089827.1:2000SEP08 | g1260513 | 763 | 922 |
| 222 | LI:1089827.1:2000SEP08 | 1329010H1 | 1558 | 1825 |
| 222 | LI:1089827.1:2000SEP08 | 2583409T6 | 170 | 291 |
| 222 | LI:1089827.1:2000SEP08 | g2522497 | 520 | 634 |
| 222 | LI:1089827.1:2000SEP08 | g2214116 | 342 | 510 |
| 222 | LI:1089827.1:2000SEP08 | g2035022 | 1 | 327 |
| 222 | LI:1089827.1:2000SEP08 | 7706257J1 | 214 | 721 |
| 222 | LI:1089827.1:2000SEP08 | 3015281H1 | 262 | 527 |
| 222 | LI:1089827.1:2000SEP08 | 4105121H1 | 420 | 512 |
| 222 | LI:1089827.1:2000SEP08 | 6866067H1 | 688 | 1269 |
| 222 | LI:1089827.1:2000SEP08 | g1639719 | 763 | 933 |
| 222 | LI:1089827.1:2000SEP08 | 60215630LI1 | 775 | 1157 |
| 222 | LI:1089827.1:2000SEP08 | 1712479H1 | 1011 | 1146 |
| 222 | LI:1089827.1:2000SEP08 | 6023176H1 | 1045 | 1322 |
| 222 | LI:1089827.1:2000SEP08 | 6728915H1 | 1180 | 1751 |
| 222 | LI:1089827.1:2000SEP08 | 2971333H1 | 1281 | 1587 |
| 222 | LI:1089827.1:2000SEP08 | 2725166T6 | 1309 | 1825 |
| 222 | LI:1089827.1:2000SEP08 | 6364173H1 | 712 | 886 |
| 222 | LI:1089827.1:2000SEP08 | g1200909 | 1403 | 1688 |
| 222 | LI:1089827.1:2000SEP08 | 2159096H1 | 1191 | 1436 |
| 222 | LI:1089827.1:2000SEP08 | 3277887H1 | 377 | 510 |
| 222 | LI:1089827.1:2000SEP08 | g1625931 | 322 | 634 |
| 222 | LI:1089827.1:2000SEP08 | g1147847 | 1550 | 1825 |
| 222 | LI:1089827.1:2000SEP08 | 3438366H1 | 233 | 487 |
| 222 | LI:1089827.1:2000SEP08 | 6142871H1 | 1369 | 1644 |
| 222 | LI:1089827.1:2000SEP08 | 2775224H1 | 801 | 1056 |
| 222 | LI:1089827.1:2000SEP08 | 2536706H1 | 1190 | 1353 |
| 222 | LI:1089827.1:2000SEP08 | 4571795H1 | 857 | 1132 |
| 222 | LI:1089827.1:2000SEP08 | 003803H1 | 1298 | 1674 |
| 222 | LI:1089827.1:2000SEP08 | 2261176H1 | 845 | 1Q73 |
| 222 | LI:1089827.1:2000SEP08 | 2261176R6 | 845 | 1105 |
| 222 | LI:1089827.1:2000SEP08 | 2464816H1 | 770 | 1013 |
| 222 | LI:1089827.1:2000SEP08 | 60215628LI1 | 801 | 1140 |
| 222 | LI:1089827.1:2000SEP08 | 4904127H2 | 578 | 799 |
| 222 | LI:1089827.1:2000SEP08 | 4771317H1 | 670 | 935 |
| 222 | LI:1089827.1:2000SEP08 | 5724326H1 | 574 | 1127 |
| 222 | LI:1089827.1:2000SEP08 | 5335354H1 | 973 | 1212 |
| 222 | LI:1089827.1:2000SEP08 | 453713H1 | 523 | 764 |
| 222 | LI:1089827.1:2000SEP08 | 2948021H1 | 232 | 432 |
| 222 | LI:1089827.1:2000SEP08 | 2231259H1 | 590 | 670 |
| 222 | LI:1089827.1:2000SEP08 | 2159096F6 | 1191 | 1650 |
| 222 | LI:1089827.1:2000SEP08 | 3437634H1 | 262 | 514 |
| 222 | LI:1089827.1:2000SEP08 | 3152883H1 | 335 | 621 |
| 222 | LI:1089827.1:2000SEP08 | 6913581J1 | 965 | 1540 |
| 222 | LI:1089827.1:2000SEP08 | 3407155H1 | 378 | 510 |
| 222 | LI:1089827.1:2000SEP08 | 6454093H1 | 841 | 1385 |
| 222 | LI:1089827.1:2000SEP08 | g2221569 | 370 | 510 |
| 222 | LI:1089827.1:2000SEP08 | 6023176F6 | 1055 | 1610 |
| 222 | LI:1089827.1:2000SEP08 | 2906529T7 | 1452 | 1762 |
| 222 | LI:1089827.1:2000SEP08 | 3246473H1 | 343 | 510 |
| 223 | LI:792112.1:2000SEP08 | 697696R6 | 595 | 928 |
| 223 | LI:792112.1:2000SEP08 | 697696T6 | 583 | 914 |
| 223 | LI:792112.1:2000SEP08 | 477786F1 | 251 | 865 |
| 223 | LI:792H2.1:2000SEP08 | 477786T6 | 433 | 830 |
| 223 | LI:792112.1:2000SEP08 | 697696H1 | 594 | 773 |
| 223 | LI:792H2.1:2000SEP08 | 5547349H1 | 358 | 577 |
| 223 | LI:792112.1:2000SEP08 | 3449269H1 | 361 | 504 |
| 223 | LI:792112.1:2000SEP08 | 1251881H1 | 111 | 367 |
| 223 | LI:792112.1:2000SEP08 | 6895912F8 | 353 | |
| 223 | LI:792112.1:2000SEP08 | 6895946F8 | 185 | 353 |
| 223 | LI:792112.1:2000SEP08 | 6895912H1 | 1 | 353 |
| 223 | LI:792112.1:2000SEP08 | 7388585H1 | 90 | 627 |
| 223 | LI:792112.1:2000SEP08 | 696573H1 | 595 | 813 |
| 223 | LI:792112.1:2000SEP08 | 3898628H1 | 1 | 241 |
| 223 | LI:792112.1:2000SEP08 | 6895946H1 | 1 | 353 |
| 223 | LI:792112.1:2000SEP08 | 4418332H1 | 149 | 394 |
| 224 | LI:282219.2:2000SEP08 | 7031 8360D1 | 1 | 281 |
| 224 | LI:282219.2:2000SEP08 | 7757121H1 | 602 | 954 |
| 224 | LI:282219.2:2000SEP08 | 70317428D1 | 619 | 910 |
| 224 | LI:282219.2:2000SEP08 | 70318919D1 | 1 | 424 |
| 224 | LI:282219.2:2000SEP08 | 2784518F6 | 7 | 470 |
| 224 | LI:282219.2:2000SEP08 | 2784518H1 | 7 | 262 |
| 224 | LI:282219.2:2000SEP08 | 70321161D1 | 157 | 542 |
| 224 | LI:282219.2:2000SEP08 | 2784518T6 | 178 | 556 |
| 224 | LI:282219.2:2000SEP08 | 4920904H1 | 208 | 491 |
| 224 | LI:282219.2:2000SEP08 | 70321204D1 | 259 | 659 |
| 224 | LI:282219.2:2000SEP08 | 70317587D1 | 259 | 467 |
| 224 | LI:282219.2:2000SEP08 | 7757121J1 | 281 | 852 |
| 224 | LI:282219.2:2000SEP08 | g4149194 | 424 | 685 |
| 225 | LI:1088010.2:2000SEP08 | g670054 | 297 | 606 |
| 225 | LI:1088010.2:2000SEP08 | g1928211 | 634 | 860 |
| 225 | LI:1088010.2:2000SEP08 | 5434205F9 | 455 | 1016 |
| 225 | LI:1088010.2:2000SEP08 | 1860333H1 | 1273 | 1482 |
| 225 | LI:1088010.2:2000SEP08 | 2229539T6 | 1214 | 1585 |
| 225 | LI:1088010.2:2000SEP08 | 2887278H1 | 642 | 915 |
| 225 | LI:1088010.2:2000SEP08 | 5434205H1 | 455 | 717 |
| 225 | LI:1088010.2:2000SEP08 | 2665463H1 | 650 | 905 |
| 225 | LI:1088010.2:2000SEP08 | 3345791H1 | 1 | 107 |
| 225 | LI:1088010.2:2000SEP08 | 60215408LI1 | 780 | 1249 |
| 225 | LI:1088010.2:2000SEP08 | g2825429 | 790 | 1207 |
| 225 | LI:1088010.2:2000SEP08 | 5728029H1 | 665 | 1031 |
| 225 | LI:1088010.2:2000SEP08 | 3487033H1 | 1 | 198 |
| 225 | LI:1088010.2:2000SEP08 | 997293H1 | 1070 | 1222 |
| 225 | LI:1088010.2:2000SEP08 | g816072 | 299 | 573 |
| 225 | LI:1088010.2:2000SEP08 | 1539214H1 | 1217 | 1440 |
| 225 | LI:1088010.2:2000SEP08 | 2838934H1 | 200 | 453 |
| 225 | LI:1088010.2:2000SEP08 | 1369182H1 | 1298 | 1564 |
| 225 | LI:1088010.2:2000SEP08 | 3703176H1 | 2 | 286 |
| 225 | LI:1088010.2:2000SEP08 | 4047723H1 | 196 | 483 |
| 225 | LI:1088010.2:2000SEP08 | 4785960H1 | 201 | 402 |
| 225 | LI:1088010.2:2000SEP08 | 4302572F6 | 276 | 727 |
| 225 | LI:1088010.2:2000SEP08 | 60215396U1 | 279 | 738 |
| 225 | LI:1088010.2:2000SEP08 | 60215398LI1 | 281 | 787 |
| 225 | LI:1088010.2:2000SEP08 | 6002309F8 | 296 | 914 |
| 225 | LI:1088010.2:2000SEP08 | 71659572V1 | 347 | 963 |
| 225 | LI:1088010.2:2000SEP08 | 71660290V1 | 383 | 1018 |
| 225 | LI:1088010.2:2000SEP08 | 60215397LI1 | 424 | 921 |
| 225 | LI:1088010.2:2000SEP08 | 60208437LI1 | 515 | 952 |
| 225 | LI:1088010.2:2000SEP08 | 60208271LI2 | 515 | 952 |
| 225 | LI:1088010.2:2000SEP08 | 60215409LI1 | 584 | 1055 |
| 225 | LI:1088010.2:2000SEP08 | 2887278F6 | 642 | 1183 |
| 225 | LI:1088010.2:2000SEP08 | 6354996F8 | 645 | 1074 |
| 225 | LI:1088010.2:2000SEP08 | 2665463F7 | 650 | 1203 |
| 225 | LI:1088010.2:2000SEP08 | g2825271 | 788 | 1219 |
| 225 | LI:1088010.2:2000SEP08 | 60215394LI1 | 772 | 1117 |
| 225 | LI:1088010.2:2000SEP08 | 5989526T8 | 994 | 1514 |
| 225 | LI:1088010.2:2000SEP08 | 696418R6 | 1007 | 1341 |
| 225 | LI:1088010.2:2000SEP08 | 71653192V1 | 1033 | 1647 |
| 225 | LI:1088010.2:2000SEP08 | 997293T6 | 1058 | 1527 |
| 225 | LI:1088010.2:2000SEP08 | 997293R6 | 1070 | 1601 |
| 225 | LI:1088010.2:2000SEP08 | 60208436LI1 | 1073 | 1609 |
| 225 | LI:1088010.2:2000SEP08 | 539754F1 | 1144 | 1629 |
| 225 | LI:1088010.2:2000SEP08 | 539754H1 | 1155 | 1376 |
| 225 | LI:1088010.2:2000SEP08 | 1662073T6 | 1149 | 1582 |
| 225 | LI:1088010.2:2000SEP08 | 539754R1 | 1156 | 1549 |
| 225 | LI:1088010.2:2000SEP08 | 2447370H1 | 1176 | 1397 |
| 225 | LI:1088010.2:2000SEP08 | 60208438LI1 | 1166 | 1606 |
| 225 | LI:1088010.2:2000SEP08 | 1932152T6 | 1220 | 1592 |
| 225 | LI:1088010.2:2000SEP08 | 1860333F6 | 1288 | 1629 |
| 225 | LI:1088010.2:2000SEP08 | 696418T6 | 1300 | 1592 |
| 225 | LI:1088010.2:2000SEP08 | g1685966 | 1457 | 1633 |
| 225 | LI:1088010.2:2000SEP08 | 2588430T6 | 1304 | 1590 |
| 225 | LI:1088010.2:2000SEP08 | 6836029H1 | 394 | 859 |
| 225 | LI:1088010.2:2000SEP08 | 2994174H1 | 527 | 827 |
| 225 | LI:1088010.2:2000SEP08 | 1369182R1 | 1298 | 1629 |
| 225 | LI:1088010.2:2000SEP08 | 4302572H1 | 278 | 529 |
| 225 | LI:1088010.2:20003EP08 | 696418H1 | 1007 | 1229 |
| 225 | LI:1088010.2:2000SEP08 | 3703176F6 | 1 | 538 |
| 225 | LI:1088010.2:2000SEP08 | 6816514J1 | 90 | 657 |
| 225 | LI:1088010.2:2000SEP08 | 6772720J1 | 112 | 624 |
| 225 | LI:1088010.2:2000SEP08 | 8010886H1 | 114 | 618 |
| 225 | LI:1088010.2:2000SEP08 | 60215401LI1 | 160 | 647 |
| 225 | LI:1088010.2:2000SEP08 | g1506103 | 637 | 925 |
| 225 | LI:1088010.2:2000SEP08 | 6002309H1 | 302 | 582 |
| 225 | LI:1088010.2:2000SEP08 | 7617283J1 | 354 | 747 |
| 225 | LI:1088010.2:2000SEP08 | 025139H1 | 1005 | 1081 |
| 225 | LI:1088010.2:2000SEP08 | 2541212H1 | 1551 | 1629 |
| 225 | LI:1088010.2:2000SEP08 | 5989526H1 | 721 | 1001 |
| 225 | LI:1088010.2:2000SEP08 | 5570460H1 | 199 | 416 |
| 225 | LI:1088010.2:2000SEP08 | 1662073H1 | 1156 | 1260 |
| 225 | LI:1088010.2:2000SEP08 | 1369182T1 | 1298 | 1588 |
| 225 | LI:1088010.2:2000SEP08 | 5801043H1 | 1034 | 1190 |
| 225 | LI:1088010.2:2000SEP08 | 60215402LI1 | 500 | 955 |
| 225 | LI:1088010.2:2000SEP08 | 60215405LI1 | 593 | 949 |
| 225 | LI:1088010.2:2000SEP08 | 999496k 1 | 1083 | 1191 |
| 225 | LI:1088010.2:2000SEP08 | 3A41343H1 | 651 | 899 |
| 225 | LI:1088010.2:2000SEP08 | 1476972T6 | 1347 | 1591 |
| 225 | LI:1088010.2:2000SEP08 | 6515741H1 | 103 | 649 |
| 225 | LI:1088010.2:2000SEP08 | 997293R2 | 1070 | 1584 |
| 225 | LI:1088010.2:2000SEP08 | g1502060 | 1346 | 1629 |
| 225 | LI:1088010.2:2000SEP08 | g1785439 | 788 | 1245 |
| 226 | LI:1165276.1:2000SEP08 | 1895443H1 | 19 | 272 |
| 226 | LI:1165276.1:2000SEP08 | 3052342H1 | 46 | 335 |
| 226 | LI:1165276.1:2000SEP08 | 3021038T6 | 568 | 880 |
| 226 | LI:1165276.1:2000SEP08 | 1891884H1 | 19 | 264 |
| 226 | LI:1165276.1:2000SEP08 | 3022533T6 | 568 | 902 |
| 226 | LI:1165276.1:2000SEP08 | 3021710H1 | 568 | 853 |
| 226 | LI:1165276.1:2000SEP08 | 4729485H1 | 86 235 | |
| 226 | LI:1165276.1:2000SEP08 | 5217490H1 | 1 | 198 |
| 226 | LI:1165276.1:2000SEP08 | 1542833H1 | 1 | 137 |
| 226 | LI:1165276.1:2000SEP08 | 3836068F6 | 7 549 | |
| 226 | LI:1165276.1:2000SEP08 | 6018614H1 | 102 | 701 |
| 226 | LI:1165276.1:2000SEP08 | g5661058 | 155 | 347 |
| 226 | LI:1165276.1:2000SEP08 | 3525414H1 | 171 | 494 |
| 226 | LI:1165276.1:2000SEP08 | 6621760H1 | 188 | 747 |
| 226 | LI:1165276.1:2000SEP08 | 3679568T9 | 546 | 925 |
| 226 | LI:1165276.1:2000SEP08 | 3836068T6 | 560 | 1044 |
| 226 | LI:1165276.1:2000SEP08 | 3021710R6 | 568 | 943 |
| 226 | LI:1165276.1:2000SEP08 | 6371853H1 | 587 | 865 |
| 226 | LI:1165276.1:2000SEP08 | 3836068H1 | 7 | 289 |
| 226 | LI:1165276.1:2000SEP08 | 5735738H1 | 78 348 | |
| 227 | LI:1169524.2:2000SEP08 | g5741053 | 237 | 456 |
| 227 | LI:1169524.2:2000SEP08 | g4983712 | 216 | 581 |
| 227 | LI:1169524.2:2000SEP08 | g4536188 | 231 | 456 |
| 227 | LI:1169524.2:2000SEP08 | 5645239H1 | 752 | 935 |
| 227 | LI:1169524.2:2000SEP08 | 6156187H1 | 1 | 322 |
| 227 | LI:1169524.2:2000SEP08 | g2583400 | 27 | 527 |
| 227 | LI:1169524.2:2000SEP08 | 505847H1 | 119 | 324 |
| 227 | LI:1169524.2:2000SEP08 | g2540081 | 125 | 581 |
| 227 | LI:1169524.2:2000SEP08 | 3015762H1 | 145 | 431 |
| 227 | LI:1169524.2:2000SEP08 | 8109677J1 | 532 | 1030 |
| 227 | LI:1169524.2:2000SEP08 | 8109677H1 | 549 | 1033 |
| 227 | LI:1169524.2:2000SEP08 | 6478543H1 | 586 | 1019 |
| 227 | LI:1169524.2:2000SEP08 | g2278640 | 151 | 581 |
| 228 | LI:1180255.1:2000SEP08 | 6946211H1 | 1102 | 1636 |
| 228 | LI:1180255.1:2000SEP08 | g2159550 | 567 | 933 |
| 228 | LI:1180255.1:2000SEP08 | g2011335 | 1381 | 1758 |
| 228 | LI:1180255.1:2000SEP08 | 5990950H1 | 1429 | 1730 |
| 228 | LI:1180255.1:2000SEP08 | 6946142H1 | 1272 | 1603 |
| 228 | LI:1180255.1:2000SEP08 | 8039729H1 | 812 | 1485 |
| 228 | LI:1180255.1:2000SEP08 | g4019078 | 498 | 942 |
| 228 | LI:1180255.1:2000SEP08 | 5286647T8 | 506 | 818 |
| 228 | LI:1180255.1:2000SEP08 | 5286647T9 | 242 | 812 |
| 228 | LI:1180255.1:2000SEP08 | 5286647F9 | 615 | |
| 228 | LI:1180255.1:2000SEP08 | 5286647F8 | 5 | 552 |
| 228 | LI:1180255.1:2000SEP08 | 5286647H1 | 6 | 163 |
| 228 | LI:1180255.1:2000SEP08 | 7725102J1 | 1074 | 1531 |
| 228 | LI:1180255.1:2000SEP08 | 5996188H1 | 1429 | 1713 |
| 229 | LI:1091903.1:2000SEP08 | 2585657H1 | 1 | 257 |
| 229 | LI:1091903.1:2000SEP08 | 5206842H2 | 1 | 215 |
| 229 | LI:1091903.1:2000SEP08 | 2587120T6 | 2 | 531 |
| 229 | LI:1091903.1:2000SEP08 | 2587120F6 | 1 | 475 |
| 229 | LI:1091903.1:2000SEP08 | g4850682 | 242 | 568 |
| 229 | LI:1091903.1:2000SEP08 | 2585657F6 | 1 | 430 |
| 229 | LI:1091903.1:2000SEP08 | 2587120H1 | 1 | 270 |
| 229 | LI:1091903.1:2000SEP08 | g4984132 | 374 | 568 |
| 230 | LI:1169219.1:2000SEP08 | 3832110H1 | 25 | 298 |
| 230 | LI:1169219.1:2000SEP08 | 2737435F6 | 19 | 501 |
| 230 | LI:1169219.1:2000SEP08 | 641592H1 | 10 | 263 |
| 230 | LI:1169219.1:2000SEP08 | g1152158 | 38 | 474 |
| 230 | LI:1169219.1:2000SEP08 | 3600686H1 | 32 | 318 |
| 230 | LI:1169219.1:2000SEP08 | 2958179H1 | 17 | 261 |
| 230 | LI:1169219.1:2000SEP08 | 2737435T6 | 420 | 617 |
| 230 | LI:1169219.1:2000SEP08 | g2031599 | 67 | 399 |
| 230 | LI:1169219.1:2000SEP08 | 5743693H1 | 65 | 356 |
| 230 | LI:1169219.1:2000SEP08 | 2737435H1 | 19 | 226 |
| 230 | LI:1169219.1:2000SEP08 | 6369702F8 | 17 | 613 |
| 230 | LI:1169219.1:2000SEP08 | 5486139H1 | 1 | 263 |
| 230 | LI:1169219.1:2000SEP08 | 3494034H1 | 14 | 284 |
| 230 | LI:1169219.1:2000SEP08 | g1924344 | 20 | 197 |
| 230 | LI:1169219.1:2000SEP08 | 3832110F8 | 28 | 683 |
| 230 | LI:1169219.1:2000SEP08 | 3600686F6 | 32 | 623 |
| 230 | LI:1169219.1:2000SEP08 | 5743693R9 | 65 | 632 |
| 230 | LI:1169219.1:2000SEP08 | 7950325J1 | 162 | 368 |
| 230 | LI:1169219.1:2000SEP08 | 7950325H1 | 162 | 370 |
| 230 | LI:1169219.1:2000SEP08 | g4072524 | 263 | 626 |
| 230 | LI:1169219.1:2000SEP08 | 3600686T6 | 293 | 920 |
| 230 | LI:1169219.1:2000SEP08 | g1087803 | 358 | 543 |
| 230 | LI:1169219.1:2000SEP08 | 5387444H1 | 20 | 287 |
| 230 | LI:1169219.1:2000SEP08 | 2550578H1 | 291 | 531 |
| 230 | LI:1169219.1:2000SEP08 | 6369702H1 | 18 | 498 |
| 230 | LI:1169219.1:2000SEP08 | 6537993H1 | 31 | 606 |
| 230 | LI:1169219.1:2000SEP08 | 651414H1 | 16 | 252 |
| 230 | LI:1169219.1:2000SEP08 | g1492368 | 28 | 219 |
| 230 | LI:1169219.1:2000SEP08 | 2939892H1 | 14 | 274 |
| 231 | LI:2050313.1:2000SEP08 | 71634215V1 | 1676 | 2208 |
| 231 | LI:2050313.1:2000SEP08 | g5920606 | 2442 | 2904 |
| 231 | LI:2050313.1:2000SEP08 | g3920192 | 2442 | 2899 |
| 231 | LI:2050313.1:2000SEP08 | g6575222 | 2444 | 2917 |
| 231 | LI:2050313.1:2000SEP08 | g5513413 | 2448 | 2907 |
| 231 | LI:2050313.1:2000SEP08 | 2460970H1 | 1803 | 2037 |
| 231 | LI:2050313.1:2000SEP08 | 517655R6 | 1 | 219 |
| 231 | LI:2050313.1:2000SEP08 | 1226309H1 | 141 | 352 |
| 231 | LI:2050313.1:2000SEP08 | g4264532 | 2551 | 2902 |
| 231 | LI:2050313.1:2000SEP08 | 108740H1 | 2552 | 2816 |
| 231 | LI:2050313.1:2000SEP08 | g888198 | 2563 | 2899 |
| 231 | LI:2050313.1:2000SEP08 | g916257 | 2536 | 2876 |
| 231 | LI:2050313.1:2000SEP08 | g3934039 | 2539 | 2912 |
| 231 | LI:2050313.1:2000SEP08 | g2354723 | 2546 | 2911 |
| 231 | LI:2050313.1:2000SEP08 | 7661242H1 | 512 | 1005 |
| 231 | LI:2050313.1:2000SEP08 | 816379H1 | 2397 | 2667 |
| 231 | LI:2050313.1:2000SEP08 | 5119740H1 | 292 | 564 |
| 231 | LI:2050313.1:2000SEP08 | 3389225H1 | 304 | 586 |
| 231 | LI:2050313.1:2000SEP08 | 1899389F6 | 1132 | 1596 |
| 231 | LI:2050313.1:2000SEP08 | 4359376H1 | 2077 | 2350 |
| 231 | LI:2050313.1:2000SEP08 | 70863583V1 | 2085 | 2646 |
| 231 | LI:2050313.1:2000SEP08 | 71190353V1 | 2088 | 2703 |
| 231 | LI:2050313.1:2000SEP08 | 3877479H1 | 1878 | 2150 |
| 231 | LI:2050313.1:2000SEP08 | 71638227V1 | 1862 | 2399 |
| 231 | LI:2050313.1:2000SEP08 | 5391812H1 | 1836 | 2134 |
| 231 | LI:2050313.1:2000SEP08 | 2438011H1 | 1828 | 2065 |
| 231 | LI:2050313.1:2000SEP08 | 5391750H1 | 1835 | 2143 |
| 231 | LI:2050313.1:2000SEP08 | 3482688H1 | 1854 | 2160 |
| 231 | LI:2050313.1:2000SEP08 | g654630 | 2494 | 2902 |
| 231 | LI:2050313.1:2000SEP08 | g6659263 | 2502 | 2905 |
| 231 | LI:2050313.1:2000SEP08 | 3727893H1 | 2017 | 2337 |
| 231 | LI:2050313.1:2000SEP08 | g5747372 | 2617 | 2890 |
| 231 | LI:2050313.1:2000SEP08 | 078994H1 | 2620 | 2896 |
| 231 | LI:2050313.1:2000SEP08 | g857160 | 2619 | 2883 |
| 231 | LI:2050313.1:2000SEP08 | 71227710V1 | 2625 | 2844 |
| 231 | LI:2050313.1:2000SEP08 | 70862890V1 | 887 | 1528 |
| 231 | LI:2050313.1:2000SEP08 | 7997537H1 | 914 | 1510 |
| 231 | LI:2050313.1:2000SEP08 | 4256662H1 | 944 | 1214 |
| 231 | LI:2050313.1:2000SEP08 | 4788606H1 | 976 | 1246 |
| 231 | LI:2050313.1:2000SEP08 | g6836105 | 3406 | 3535 |
| 231 | LI:2050313.1:2000SEP08 | g2278442 | 2586 | 2909 |
| 231 | LI:2050313.1:2000SEP08 | g2238769 | 2489 | 2917 |
| 231 | LI:2050313.1:2000SEP08 | 70860996V1 | 1773 | 2367 |
| 231 | LI:2050313.1:2000SEP08 | 71191664V1 | 1778 | 2246 |
| 231 | LI:2050313.1:2000SEP08 | 70861786V1 | 1786 | 2485 |
| 231 | LI:2050313.1:2000SEP08 | 71227914V1 | 867 | 1448 |
| 231 | LI:2050313.1:2000SEP08 | 71226748V1 | 887 | 1481 |
| 231 | LI:2050313.1:2000SEP08 | 3645781F6 | 887 | 1219 |
| 231 | LI:2050313.1:2000SEP08 | 3645781H1 | 887 | 1181 |
| 231 | LI:2050313.1:2000SEP08 | 978178R6 | 2528 | 2911 |
| 231 | LI:2050313.1:2000SEP08 | 978178T1 | 2528 | 2864 |
| 231 | LI:2050313.1:2000SEP08 | 978178T6 | 2528 | 2852 |
| 231 | LI:2050313.1:2000SEP08 | 70861724V1 | 1552 | 2187 |
| 231 | LI:2050313.1:2000SEP08 | 70864348V1 | 1589 | 2199 |
| 231 | LI:2050313.1:2000SEP08 | 70863732V1 | 1639 | 2322 |
| 231 | LI:2050313.1:2000SEP08 | 6330188H1 | 1648 | 2158 |
| 231 | LI:2050313.1:2000SEP08 | 71639036V1 | 1674 | 2245 |
| 231 | LI:2050313.1:2000SEP08 | g1760607 | 1992 | 2359 |
| 231 | LI:2050313.1:2000SEP08 | 71191657V1 | 1992 | 2629 |
| 231 | LI:2050313.1:2000SEP08 | 7659719J1 | 1994 | 2665 |
| 231 | LI:2050313.1:2000SEP08 | g4889106 | 3344 | 3563 |
| 231 | LI:2050313.1:2000SEP08 | 70697058V1 | 2435 | 2991 |
| 231 | LI:2050313.1:2000SEP08 | 70861844V1 | 1946 | 2492 |
| 231 | LI:2050313.1:2000SEP08 | 2800074H1 | 1946 | 2241 |
| 231 | LI:2050313.1:20003EP08 | g857159 | 1958 | 2269 |
| 231 | LI:2050313.1:2000SEP08 | g866480 | 1958 | 2280 |
| 231 | LI:2050313.1:2000SEP08 | 4778417H1 | 2979 | 3263 |
| 231 | LI:2050313.1:2000SEP08 | g4373468 | 3070 | 3544 |
| 231 | LI:2050313.1:2000SEP08 | 4903502H2 | 2864 | 2921 |
| 231 | LI:2050313.1:2000SEP08 | 5722778H1 | 2898 | 3447 |
| 231 | LI:2050313.1:2000SEP08 | 70696758V1 | 2231 | 2506 |
| 231 | LI:2050313.1:2000SEP08 | 4694505H1 | 2232 | 2431 |
| 231 | LI:2050313.1:2000SEP08 | 7063383H1 | 2471 | 2801 |
| 231 | LI:2050313.1:2000SEP08 | 71635157V1 | 2473 | 3045 |
| 231 | LI:2050313.1:2000SEP08 | 2079269T6 | 2481 | 2851 |
| 231 | LI:2050313.1:2000SEP08 | 2130543H1 | 2454 | 2737 |
| 231 | LI:2050313.1:2000SEP08 | 70694490V1 | 2458 | 2927 |
| 231 | LI:2050313.1:2000SEP08 | g2220430 | 2465 | 2911 |
| 231 | LI:2050313.1:2000SEP08 | g1928342 | 2487 | 2884 |
| 231 | LI:2050313.1:2000SEP08 | 816379T6 | 2435 | 2852 |
| 231 | LI:2050313.1:2000SEP08 | 5119740F6 | 292 | 753 |
| 231 | LI:2050313.1:2000SEP08 | g1970036 | 1817 | 2199 |
| 231 | LI:2050313.1:2000SEP08 | g3095393 | 2522 | 2913 |
| 231 | LI:2050313.1:2000SEP08 | g866395 | 2517 | 2882 |
| 231 | LI:2050313.1:2000SEP08 | 517655H1 | 1 | 226 |
| 231 | LI:2050313.1:2000SEP08 | 2012964H1 | 1982 | 2271 |
| 231 | LI:2050313.1:2000SEP08 | g1928165 | 1983 | 2430 |
| 231 | LI:2050313.1:2000SEP08 | 3742339H1 | 1986 | 2296 |
| 231 | LI:2050313.1:2000SEP08 | g823926 | 2614 | 2924 |
| 231 | LI:2050313.1:2000SEP08 | 1899389H1 | 1132 | 1371 |
| 231 | LI:2050313.1:2000SEP08 | 70864752V1 | 1137 | 1586 |
| 231 | LI:2050313.1:2000SEP08 | 71228173V1 | 1160 | 1709 |
| 231 | LI:2050313.1:2000SEP08 | 4572460H1 | 1167 | 1458 |
| 231 | LI:2050313.1:2000SEP08 | 835906H1 | 1802 | 2105 |
| 231 | LI:2050313.1:2000SEP08 | 5460718H1 | 1122 | 1379 |
| 231 | LI:2050313.1:2000SEP08 | g6398687 | 2432 | 2899 |
| 231 | LI:2050313.1:2000SEP08 | g2669938 | 2432 | 2899 |
| 231 | LI:2050313.1:2000SEP08 | 71188286V1 | 1885 | 2447 |
| 231 | LI:2050313.1:2000SEP08 | g2878831 | 2652 | 2907 |
| 231 | LI:2050313.1:2000SEP08 | g1548944 | 2664 | 2913 |
| 231 | LI:2050313.1:2000SEP08 | g2360009 | 2675 | 2902 |
| 231 | LI:2050313.1:2000SEP08 | g5837028 | 2427 | 2917 |
| 231 | LI:2050313.1:2000SEP08 | 6011505H1 | 2425 | 2729 |
| 231 | LI:2050313.1:2000SEP08 | 71637073V1 | 1676 | 2206 |
| 231 | LI:2050313.1:2000SEP08 | 3408725F6 | 1676 | 2141 |
| 231 | LI:2050313.1:2000SEP08 | g2008872 | 1741 | 2031 |
| 231 | LI:2050313.1:2000SEP08 | 71638790V1 | 1774 | 2386 |
| 231 | LI:2050313.1:2000SEP08 | 71636016V1 | 1676 | 2160 |
| 231 | LI:2050313.1:2000SEP08 | 7250342H1 | 1922 | 2471 |
| 231 | LI:2050313.1:2000SEP08 | 71188468V1 | 1916 | 2546 |
| 231 | LI:2050313.1:2000SEP08 | g2069853 | 2502 | 2900 |
| 231 | LI:2050313.1:2000SEP08 | g1920687 | 1413 | 1782 |
| 231 | LI:2050313.1:2000SEP08 | 6805456J1 | 1223 | 1483 |
| 231 | LI:2050313.1:2000SEP08 | 70857067V1 | 1415 | 2077 |
| 231 | LI:2050313.1:2000SEP08 | 2307192H1 | 1238 | 1341 |
| 231 | LI:2050313.1:2000SEP08 | 70863071V1 | 1250 | 1855 |
| 231 | LI:2050313.1:2000SEP08 | 7964809H1 | 1264 | 1818 |
| 231 | LI:2050313.1:2000SEP08 | 70863112V1 | 1274 | 1536 |
| 231 | LI:2050313.1:2000SEP08 | 6154114H1 | 1277 | 1622 |
| 231 | LI:2050313.1:2000SEP08 | 71227225V1 | 1399 | 1881 |
| 231 | LI:2050313.1:2000SEP08 | 70864328V1 | 1423 | 2050 |
| 231 | LI:2050313.1:2000SEP08 | g4901359 | 3100 | 3548 |
| 231 | LI:2050313.1:2000SEP08 | 1809247F6 | 3105 | 3548 |
| 231 | LI:2050313.1:2000SEP08 | 1809247H1 | 3105 | 3361 |
| 231 | LI:2050313.1:2000SEP08 | g3932181 | 3117 | 3564 |
| 231 | LI:2050313.1:2000SEP08 | 7220548H1 | 619 | 1139 |
| 231 | LI:2050313.1:2000SEP08 | 3406852H1 | 2050 | 2346 |
| 231 | LI:2050313.1:2000SEP08 | 5850271H1 | 2017 | 2327 |
| 231 | LI:2050313.1:2000SEP08 | 70864279V1 | 2032 | 2781 |
| 231 | LI:2050313.1:2000SEP08 | g2882152 | 2565 | 2876 |
| 231 | LI:2050313.1:2000SEP08 | 3114939H1 | 1043 | 1317 |
| 231 | LI:2050313.1:2000SEP08 | 2724324H1 | 2534 | 2805 |
| 231 | LI:2050313.1:2000SEP08 | 71189866V1 | 2393 | 2899 |
| 231 | LI:2050313.1:2000SEP08 | 816324R1 | 2424 | 2899 |
| 231 | LI:2050313.1:2000SEP08 | 816379R6 | 2424 | 2887 |
| 231 | LI:2050313.1:2000SEP08 | 816324T1 | 2424 | 2853 |
| 231 | LI:2050313.1:2000SEP08 | 816324H1 | 2424 | 2698 |
| 231 | LI:2050313.1:2000SEP08 | 71634142V1 | 1696 | 2208 |
| 231 | LI:2050313.1:2000SEP08 | 71637537V1 | 1722 | 2254 |
| 231 | LI:2050313.1:2000SEP08 | 71635238V1 | 1742 | 2280 |
| 231 | LI:2050313.1:2000SEP08 | 71635209V1 | 1676 | 2057 |
| 231 | LI:2050313.1:2000SEP08 | 3408725H1 | 1676 | 1935 |
| 231 | LI:2050313.1:2000SEP08 | 70861652V1 | 1744 | 2174 |
| 231 | LI:2050313.1:2000SEP08 | 7934412H1 | 1208 | 1520 |
| 231 | LI:2050313.1:2000SEP08 | 6805456H1 | 1224 | 1492 |
| 231 | LI:2050313.1:2000SEP08 | 6346232H1 | 1923 | 2244 |
| 231 | LI:2050313.1:2000SEP08 | 6350852H1 | 1923 | 2225 |
| 231 | LI:2050313.1:2000SEP08 | 6345894H1 | 1923 | 2134 |
| 231 | LI:2050313.1:2000SEP08 | 5206567H1 | 2526 | 2812 |
| 231 | LI:2050313.1:2000SEP08 | 6573351H1 | 1931 | 2637 |
| 231 | LI:2050313.1:2000SEP08 | 6408556H1 | 1935 | 2544 |
| 231 | LI:2050313.1:2000SEP08 | 71636725V1 | 1937 | 2493 |
| 231 | LI:2050313.1:2000SEP08 | 71635529V1 | 1976 | 2483 |
| 231 | LI:2050313.1:2000SEP08 | 71188109V1 | 1986 | 2543 |
| 231 | LI:2050313.1:2000SEP08 | 71131715V1 | 1968 | 2360 |
| 231 | LI:2050313.1:2000SEP08 | 71228144V1 | 1971 | 2612 |
| 231 | LI:2050313.1:2000SEP08 | 71188369V1 | 1985 | 2550 |
| 231 | LI:2050313.1:2000SEP08 | 6125205H1 | 2001 | 2334 |
| 231 | LI:2050313.1:2000SEP08 | 2012964R6 | 1982 | 2488 |
| 231 | LI:2050313.1:2000SEP08 | 978178H1 | 2528 | 2887 |
| 231 | LI:2050313.1:2000SEP08 | 978178R1 | 2528 | 2911 |
| 231 | LI:2050313.1:2000SEP08 | 5078756F8 | 3130 | 3194 |
| 231 | LI:2050313.1:2000SEP08 | 661316T6 | 3172 | 3369 |
| 231 | LI:2050313.1:2000SEP08 | g4080776 | 3211 | 3670 |
| 231 | LI:2050313.1:2000SEP08 | 151807H1 | 3224 | 3477 |
| 231 | LI:2050313.1:2000SEP08 | 71228668V1 | 1176 | 1742 |
| 231 | LI:2050313.1:2000SEP08 | 71226830V1 | 2093 | 2253 |
| 231 | LI:2050313.1:2000SEP08 | 71227621V1 | 2093 | 2251 |
| 231 | LI:2050313.1:2000SEP08 | 70696141V1 | 2226 | 2860 |
| 231 | LI:2050313.1:2000SEP08 | 1879914H1 | 379 | 641 |
| 231 | LI:2050313.1:2000SEP08 | 3105961H1 | 473 | 712 |
| 231 | LI:2050313.1:2000SEP08 | 8008695H1 | 1063 | 1709 |
| 231 | LI:2050313.1:2000SEP08 | 71607269V1 | 2571 | 2899 |
| 231 | LI:2050313.1:2000SEP08 | g1760342 | 2585 | 2904 |
| 231 | LI:2050313.1:2000SEP08 | g565984 | 2576 | 2909 |
| 231 | LI:2050313.1:2000SEP08 | 4592624H1 | 1079 | 1312 |
| 231 | LI:2050313.1:2000SEP08 | 71228271V1 | 1108 | 1687 |
| 231 | LI:2050313.1:2000SEP08 | 70862220V1 | 1113 | 1682 |
| 231 | LI:2050313.1:2000SEP08 | 70862017V1 | 1125 | 1779 |
| 231 | LI:2050313.1:2000SEP08 | 7933078H1 | 2580 | 2927 |
| 231 | LI:2050313.1:2000SEP08 | 3770377H1 | 256.4 | 2864 |
| 231 | LI:2050313.1:2000SEP08 | 71635712V1 | 1676 | 2158 |
| 231 | LI:2050313.1:2000SEP08 | 1899389T6 | 2627 | 2866 |
| 231 | LI:2050313.1:2000SEP08 | g1512825 | 2651 | 2903 |
| 231 | LI:2050313.1:2000SEP08 | 70693074V1 | 2429 | 3040 |
| 231 | LI:2050313.1:2000SEP08 | g5233507 | 2417 | 2540 |
| 231 | LI:2050313.1:2000SEP08 | 4692958H1 | 2232 | 2516 |
| 231 | LI:2050313.1:2000SEP08 | 3328577H1 | 1795 | 2078 |
| 231 | LI:2050313.1:2000SEP08 | 70860945V1 | 1786 | 2196 |
| 231 | LI:2050313.1:2000SEP08 | g654629 | 2403 | 2524 |
| 231 | LI:2050313.1:2000SEP08 | g1860187 | 2535 | 2899 |
| 231 | LI:2050313.1:2000SEP08 | 3316892H1 | 182 | 440 |
| 231 | LI:2050313.1:2000SEP08 | 1956917H1 | 2840 | 3119 |
| 231 | LI:2050313.1:2000SEP08 | 70695934V1 | 2446 | 2899 |
| 231 | LI:2050313.1:2000SEP08 | 71637227V1 | 1980 | 2627 |
| 231 | LI:2050313.1:2000SEP08 | 4960572H1 | 1962 | 2253 |
| 231 | LI:2050313.1:2000SEP08 | 3111414H1 | 521 | 796 |
| 231 | LI:2050313.1:2000SEP08 | 7987068H1 | 549 | 1163 |
| 231 | LI:2050313.1:2000SEP08 | 4369673H1 | 2233 | 2529 |
| 231 | LI:2050313.1:2000SEP08 | 71634030V1 | 2275 | 2908 |
| 231 | LI:2050313.1:2000SEP08 | 71227875V1 | 2318 | 2985 |
| 231 | LI:2050313.1:2000SEP08 | 3408725T6 | 2248 | 2849 |
| 231 | LI:2050313.1:2000SEP08 | 71188660V1 | 2349 | 2917 |
| 231 | LI:2050313.1:2000SEP08 | 1361723T6 | 2346 | 2860 |
| 231 | LI:2050313.1:2000SEP08 | 3660056T6 | 2362 | 2850 |
| 231 | LI:2050313.1:20003EP08 | 70695041V1 | 2363 | 2704 |
| 231 | LI:2050313.1:2000SEP08 | 4338315H1 | 2257 | 2576 |
| 231 | LI:2050313.1:2000SEP08 | 2082382H1 | 2259 | 2524 |
| 231 | LI:2050313.1:2000SEP08 | g5755413 | 2375 | 2913 |
| 231 | LI:2050313.1:2000SEP08 | 70692978V1 | 2378 | 2899 |
| 231 | LI:2050313.1:2000SEP08 | 4307104H1 | 2261 | 2630 |
| 231 | LI:2050313.1:2000SEP08 | 70698490V1 | 2269 | 2387 |
| 231 | LI:2050313.1:2000SEP08 | 71224865V1 | 2278 | 2431 |
| 231 | LI:2050313.1:2000SEP08 | 4737021H1 | 2288 | 2597 |
| 231 | LI:2050313.1:2000SEP08 | 71633989V1 | 2312 | 2891 |
| 231 | LI:2050313.1:2000SEP08 | 71132191V1 | 2305 | 2761 |
| 231 | LI:2050313.1:2000SEP08 | 70696873V1 | 2379 | 2899 |
| 231 | LI:2050313.1:2000SEP08 | 71634146V1 | 2414 | 2899 |
| 231 | LI:2050313.1:2000SEP08 | g1801984 | 2781 | 2877 |
| 231 | LI:2050313.1:2000SEP08 | g756063 | 2694 | 2885 |
| 231 | LI:2050313.1:2000SEP08 | g2987420 | 2739 | 2908 |
| 231 | LI:2050313.1:2000SEP08 | g1860015 | 2506 | 2903 |
| 231 | LI:2050313.1:2000SEP08 | 6433329H1 | 2510 | 2900 |
| 231 | LI:2050313.1:2000SEP08 | 7641341J1 | 3458 | 4174 |
| 231 | LI:2050313.1:2000SEP08 | 6105823H1 | 3467 | 3740 |
| 231 | LI:2050313.1:2000SEP08 | g2910706 | 3585 | 3740 |
| 231 | LI:2050313.1:2000SEP08 | g2910739 | 3585 | 4086 |
| 231 | LI:2050313.1:2000SEP08 | 4661008H1 | 3651 | 3740 |
| 231 | LI:2050313.1:2000SEP08 | 4659318H1 | 3656 | 3740 |
| 231 | LI:2050313.1:2000SEP08 | g4080775 | 3218 | 3670 |
| 231 | LI:2050313.1:2000SEP08 | 71189583V1 | 2103 | 2653 |
| 231 | LI:2050313.1:2000SEP08 | 70696248V1 | 2161 | 2886 |
| 231 | LI:2050313.1:2000SEP08 | 2469908H1 | 2169 | 2466 |
| 231 | LI:2050313.1:2000SEP08 | 1721978T6 | 2183 | 2852 |
| 231 | LI:2050313.1:2000SEP08 | 2012964T6 | 2186 | 2864 |
| 231 | LI:2050313.1:2000SEP08 | 6545796F8 | 2194 | 2910 |
| 231 | LI:2050313.1:2000SEP08 | 6545396H1 | 2195 | 2830 |
| 231 | LI:2050313.1:2000SEP08 | 71189025V1 | 2215 | 2851 |
| 231 | LI:2050313.1:2000SEP08 | 71228669V1 | 1424 | 1950 |
| 231 | LI:2050313.1:2000SEP08 | 70860849V1 | 1425 | 2100 |
| 231 | LI:2050313.1:2000SEP08 | 71225347V1 | 1478 | 1780 |
| 231 | LI:2050313.1:2000SEP08 | 70856757V1 | 1478 | 1779 |
| 231 | LI:2050313.1:2000SEP08 | 1508317H1 | 3096 | 3275 |
| 231 | LI:2050313.1:2000SEP08 | 1809247T6 | 3098 | 3516 |
| 231 | LI:2050313.1:2000SEP08 | g3076640 | 3100 | 3545 |
| 231 | LI:2050313.1:2000SEP08 | 6057569H1 | 2056 | 2563 |
| 231 | LI:2050313.1:2000SEP08 | 2664547H1 | 2058 | 2315 |
| 231 | LI:2050313.1:2000SEP08 | 4887935H1 | 708 | 960 |
| 231 | LI:2050313.1:2000SEP08 | g5365501 | 2435 | 2902 |
| 232 | LI:209351.3:2000SEP08 | 70721160V1 | 1388 | 1560 |
| 232 | LI:209351.3:2000SEP08 | 60108516D4 | 1 | 528 |
| 232 | LI:209351.3:2000SEP08 | g900310 | 158 | 491 |
| 232 | LI:209351.3:2000SEP08 | 70155590V1 | 361 | 933 |
| 232 | LI:209351.3:2000SEP08 | 70157745V1 | 395 | 915 |
| 232 | LI:209351.3:2000SEP08 | 70154454V1 | 427 | 973 |
| 232 | LI:209351.3:2000SEP08 | 70151782V1 | 995 | 1409 |
| 232 | LI:209351.3:20003EP08 | 70151494V1 | 995 | 1291 |
| 232 | LI:209351.3:2000SEP08 | 70154238V1 | 1002 | 1378 |
| 232 | LI:209351.3:2000SEP08 | 70716405V1 | 1005 | 1356 |
| 232 | LI:209351.3:2000SEP08 | 70155484V1 | 1008 | 1479 |
| 232 | LI:209351.3:2000SEP08 | 70716955V1 | 1011 | 1213 |
| 232 | LI:209351.3:2000SEP08 | 1553983F6 | 1013 | 1364 |
| 232 | LI:209351.3:2000SEP08 | 70158094V1 | 1014 | 1447 |
| 232 | LI:209351.3:2000SEP08 | 1553983H1 | 1013 | 1201 |
| 232 | LI:209351.3:2000SEP08 | 70159779V1 | 1025 | 1432 |
| 232 | LI:209351.3:2000SEP08 | 2137838H1 | 1027 | 1195 |
| 232 | LI:209351.3:2000SEP08 | 70715669V1 | 1032 | 1278 |
| 232 | LI:209351.3:2000SEP08 | 70717622V1 | 1063 | 1691 |
| 232 | LI:209351.3:2000SEP08 | 70158357V1 | 934 | 1420 |
| 232 | LI:209351.3:2000SEP08 | 6010851682 | 951 | 1414 |
| 232 | LI:209351.3:2000SEP08 | 7071167H1 | 953 | 1175 |
| 232 | LI:209351.3:2000SEP08 | g711226 | 972 | 1062 |
| 232 | LI:209351.3:2000SEP08 | 70159097V1 | 977 | 1278 |
| 232 | LI:209351.3:2000SEP08 | 70160136V1 | 985 | 1447 |
| 232 | LI:209351.3:2000SEP08 | g1979986 | 990 | 1298 |
| 232 | LI:209351.3:2000SEP08 | 70151590V1 | 995 | 1460 |
| 232 | LI:209351.3:2000SEP08 | 2857819H1 | 1977 | 2263 |
| 232 | LI:209351.3:2000SEP08 | 70717097V1 | 1917 | 2304 |
| 232 | LI:209351.3:2000SEP08 | 70160951 V1 | 685 | 999 |
| 232 | LI:209351.3:2000SEP08 | 4376488H1 | 685 | 947 |
| 232 | LI:209351.3:2000SEP08 | 60108513D2 | 1 | 362 |
| 232 | LI:209351.3:2000SEP08 | 60108516D2 | 1 | 268 |
| 232 | LI:209351.3:2000SEP08 | 6010851684 | 926 | 1387 |
| 232 | LI:209351.3:2000SEP08 | 70160330V1 | 923 | 1437 |
| 232 | LI:209351.3:2000SEP08 | g793215 | 1296 | 1667 |
| 232 | LI:209351.3:2000SEP08 | 804128411 | 1447 | 2172 |
| 232 | LI:209351.3:2000SEP08 | 70159213V1 | 1 | 479 |
| 232 | LI:209351.3:2000SEP08 | 70152537V1 | 1310 | 1437 |
| 232 | LI:209351.3:2000SEP08 | 70714105V1 | 1353 | 1596 |
| 232 | LI:209351.3:2000SEP08 | 70718058V1 | 1373 | 1608 |
| 232 | LI:209351.3:2000SEP08 | 70710618V1 | 1301 | 1488 |
| 232 | LI:209351.3:2000SEP08 | 70157863V1 | 443 | 914 |
| 232 | LI:209351.3:2000SEP08 | 70719306V1 | 751 | 1108 |
| 232 | LI:209351.3:2000SEP08 | 3327158H1 | 648 | 928 |
| 232 | LI:209351.3:2000SEP08 | 70152286V1 | 745 | 1333 |
| 232 | LI:209351.3:2000SEP08 | 267142H1 | 188 | 610 |
| 232 | LI:209351.3:2000SEP08 | 70159211V1 | 214 | 735 |
| 232 | LI:209351.3:2000SEP08 | 5301713H1 | 2106 | 2373 |
| 232 | LI:209351.3:2000SEP08 | 4500146H1 | 1495 | 1771 |
| 232 | LI:209351.3:2000SEP08 | 7627889J1 | 1515 | 2130 |
| 232 | LI:209351.3:2000SEP08 | 70720925V1 | 1519 | 2144 |
| 232 | LI:209351.3:2000SEP08 | 70152094V1 | 744 | 1337 |
| 232 | LI:209351.3:2000SEP08 | 70717830V1 | 1184 | 1826 |
| 232 | LI:209351.3:2000SEP08 | 70151972V1 | 1199 | 1447 |
| 232 | LI:209351.3:2000SEP08 | 70151488V1 | 1069 | 1448 |
| 232 | LI:209351.3:2000SEP08 | 70160399V1 | 1070 | 1465 |
| 232 | LI:209351.3:2000SEP08 | 70157718V1 | 1079 | 1465 |
| 232 | LI:209351.3:2000SEP08 | 70156234V1 | 1081 | 1435 |
| 232 | LI:209351.3:2000SEP08 | 70152471V1 | 1087 | 1454 |
| 232 | LI:209351.3:2000SEP08 | 70157391V1 | 1107 | 1458 |
| 232 | LI:209351.3:2000SEP08 | 70161681 V1 | 1116 | 1447 |
| 232 | LI:209351.3:2000SEP08 | 70156138V1 | 1118 | 1462 |
| 232 | LI:209351.3:2000SEP08 | 70156038V1 | 1123 | 1462 |
| 232 | LI:209351.3:2000SEP08 | 70719134V1 | 1195 | 1590 |
| 232 | LI:209351.3:2000SEP08 | 71273545V1 | 2074 | 2675 |
| 232 | LI:209351.3:2000SEP08 | 6623018J1 | 708 | 1308 |
| 232 | LI:209351.3:2000SEP08 | 70151903V1 | 685 | 1170 |
| 232 | LI:209351.3:2000SEP08 | g1988613 | 740 | 1049 |
| 232 | LI:209351.3:2000SEP08 | 70151788V1 | 845 | 1389 |
| 232 | LI:209351.3:2000SEP08 | 70157541V1 | 875 | 1294 |
| 232 | LI:209351.3:2000SEP08 | 70712620V1 | 880 | 1218 |
| 232 | LI:209351.3:2000SEP08 | 70689377V1 | 883 | 1488 |
| 232 | LI:209351.3:2000SEP08 | 70151840V1 | 899 | 1361 |
| 232 | LI:209351.3:2000SEP08 | g713726 | 903 | 1201 |
| 232 | LI:209351.3:2000SEP08 | 70720502V1 | 1590 | 2144 |
| 232 | LI:209351.3:2000SEP08 | 70717624V1 | 1625 | 1804 |
| 232 | LI:209351.3:2000SEP08 | 70717872V1 | 1649 | 2012 |
| 232 | LI:209351.3:2000SEP08 | 70718549V1 | 1649 | 1773 |
| 232 | LI:209351.3:2000SEP08 | 6993359H1 | 1575 | 2193 |
| 232 | LI:209351.3:2000SEP08 | 70717405V1 | 1590 | 2079 |
| 232 | LI:209351.3:2000SEP08 | 70647862V1 | 1700 | 2090 |
| 232 | LI:209351.3:2000SEP08 | 70717416V1 | 755 | 1280 |
| 232 | LI:209351.3:2000SEP08 | 70159034V1 | 273 | 735 |
| 232 | LI:209351.3:2000SEP08 | 70158626V1 | 811 | 1316 |
| 232 | LI:209351.3:2000SEP08 | 71271703V1 | 2199 | 2776 |
| 232 | LI:209351.3:2000SEP08 | 2402824H1 | 2212 | 2442 |
| 232 | LI:209351.3:2000SEP08 | 71271047V1 | 2296 | 2603 |
| 232 | LI:209351.3:2000SEP08 | 71272430V1 | 2413 | 2603 |
| 232 | LI:209351.3:2000SEP08 | 4500146T6 | 2522 | 2977 |
| 232 | LI:209351.3:2000SEP08 | 1377137H1 | 2728 | 2927 |
| 232 | LI:209351.3:2000SEP08 | 5193988H1 | 2111 | 2376 |
| 232 | LI:209351.3:2000SEP08 | 1876631H1 | 2135 | 2409 |
| 232 | LI:209351.3:2000SEP08 | 71272644V1 | 2148 | 2603 |
| 232 | LI:209351.3:2000SEP08 | 70912859V1 | 2166 | 2650 |
| 232 | LI:209351.3:2000SEP08 | 2255236H1 | 2182 | 2410 |
| 232 | LI:209351.3:2000SEP08 | 70158340V1 | 761 | 1315 |
| 232 | LI:209351.3:2000SEP08 | 70160323V1 | 774 | 1173 |
| 232 | LI:209351.3:2000SEP08 | 70151309V1 | 485 | 903 |
| 232 | LI:209351.3:2000SEP08 | 6482421H1 | 605 | 1155 |
| 232 | LI:209351.3:2000SEP08 | 70716723V1 | 1200 | 1399 |
| 232 | LI:209351.3:2000SEP08 | 70720944V1 | 1200 | 1399 |
| 232 | LI:209351.3:2000SEP08 | 70157680V1 | 1 | 458 |
| 232 | LI:209351.3:2000SEP08 | 70154364V1 | 1 | 410 |
| 232 | LI:209351.3:2000SEP08 | 8123890H1 | 183 | 851 |
| 232 | LI:209351.3:2000SEP08 | 70160906V1 | 1 | 442 |
| 232 | LI:209351.3:2000SEP08 | 70152279V1 | 775 | 1455 |
| 232 | LI:209351.3:2000SEP08 | 4500146F6 | 1494 | 1814 |
| 232 | LI:209351.3:2000SEP08 | 70716459V1 | 1544 | 1914 |
| 232 | LI:209351.3:2000SEP08 | 3255709H1 | 2086 | 2344 |
| 232 | LI:209351.3:2000SEP08 | 5301828H1 | 2106 | 2373 |
| 232 | LI:209351.3:2000SEP08 | 70161870V1 | 1 | 218 |
| 232 | LI:209351.3:2000SEP08 | g673230 | 156 | 464 |
| 232 | LI:209351.3:2000SEP08 | 70718810V1 | 1176 | 1521 |
| 232 | LI:209351.3:2000SEP08 | 6010851382 | 1182 | 1406 |
| 232 | LI:209351.3:2000SEP08 | 70715834V1 | 1427 | 1974 |
| 232 | LI:209351.3:2000SEP08 | 7091 1910V1 | 2015 | 2551 |
| 232 | LI:209351.3:2000SEP08 | 70914639V1 | 2023 | 2608 |
| 232 | LI:209351.3:2000SEP08 | 71271313V1 | 2024 | 2603 |
| 232 | LI:209351.3:2000SEP08 | 2257709H1 | 2057 | 2316 |
| 232 | LI:209351.3:2000SEP08 | 70913790V1 | 2001 | 2514 |
| 232 | LI:209351.3:2000SEP08 | 2137158F6 | 1980 | 2454 |
| 232 | LI:209351.3:2000SEP08 | 2137158H1 | 1980 | 2232 |
| 232 | LI:209351.3:2000SEP08 | 70717485V1 | 1807 | 2348 |
| 232 | LI:209351.3:2000SEP08 | g2032832 | 1909 | 2262 |
| 232 | LI:209351.3:2000SEP08 | 7091 1075V1 | 1700 | 2271 |
| 232 | LI:209351.3:2000SEP08 | 70716432V1 | 1726 | 2302 |
| 232 | LI:209351.3:2000SEP08 | 70718263V1 | 1756 | 2199 |
| 232 | LI:209351.3:2000SEP08 | 70715831 V1 | 1756 | 2169 |
| 232 | LI:209351.3:2000SEP08 | 70716717V1 | 1791 | 2236 |
| 232 | LI:209351.3:2000SEP08 | 70716125V1 | 1803 | 2348 |
| 232 | LI:209351.3:2000SEP08 | 70153905V1 | 1206 | 1436 |
| 232 | LI:209351.3:2000SEP08 | g2563746 | 1225 | 1547 |
| 232 | LI:209351.3:2000SEP08 | 5774651H1 | 1230 | 1826 |
| 232 | LI:209351.3:2000SEP08 | 2864359F6 | 755 | 1165 |
| 232 | LI:209351.3:2000SEP08 | 2864359H1 | 755 | 1059 |
| 233 | LI:119900.1:2000SEP08 | 2616951T6 | 428 | 822 |
| 233 | LI:119900.1:2000SEP08 | 2831669T6 | 513 | 815 |
| 233 | LI:119900.1:2000SEP08 | 3255201H1 | 50 | 305 |
| 233 | LI:119900.1:2000SEP08 | 6205811H1 | 75 | 642 |
| 233 | LI:119900.1:2000SEP08 | g6710166 | 403 | 854 |
| 233 | LI:119900.1:2000SEP08 | 6131670H1 | 37 | 327 |
| 233 | LI:119900.1:2000SEP08 | 3255201R6 | 49 | 278 |
| 233 | LI:119900.1:2000SEP08 | g1259907 | 1 | 258 |
| 233 | LI:119900.1:2000SEP08 | 41440983 | 1 | 328 |
| 233 | LI:119900.1:2000SEP08 | 6131670F8 | 37 | 470 |
| 233 | LI:119900.1:2000SEP08 | 2423774H1 | 604 | 828 |
| 233 | LI:119900.1:2000SEP08 | g6639199 | 638 | 841 |
| 233 | LI:119900.1:2000SEP08 | 2831669H1 | 520 | 787 |
| 233 | LI:119900.1:2000SEP08 | g2437451 | 571 | 854 |
| 233 | LI:119900.1:2000SEP08 | 599026H1 | 644 | 854 |
| 233 | LI:119900.1:2000SEP08 | 5077681H1 | 737 | 854 |
| 233 | LI:119900.1:2000SEP08 | 2831669F6 | 520 | 854 |
| 234 | LI:2052274.1:2000SEP08 | 71742269V1 | 355 | 819 |
| 234 | LI:2052274.1:2000SEP08 | 71747037V1 | 890 | 1276 |
| 234 | LI:2052274.1:2000SEP08 | 7318950H2 | 1212 | 1722 |
| 234 | LI:2052274.1:2000SEP08 | g766967 | 1227 | 1590 |
| 234 | LI:2052274.1:2000SEP08 | 2797262T6 | 1282 | 1732 |
| 234 | LI:2052274.1:2000SEP08 | 71742947V1 | 1071 | 1509 |
| 234 | LI:2052274.1:2000SEP08 | 71744365V1 | 1154 | 1687 |
| 234 | LI:2052274.1:2000SEP08 | 3732853H1 | 1138 | 1288 |
| 234 | LI:2052274.1:2000SEP08 | 7318884H2 | 1212 | 1732 |
| 234 | LI:2052274.1:2000SEP08 | 877185H1 | 341 | 601 |
| 234 | LI:2052274.1:2000SEP08 | 5674936T6 | 858 | 1398 |
| 234 | LI:2052274.1:2000SEP08 | 71741876V1 | 885 | 1417 |
| 234 | LI:2052274.1:2000SEP08 | 71742240V1 | 1008 | 1509 |
| 234 | LI:2052274.1:2000SEP08 | 71746493V1 | 1016 | 1530 |
| 234 | LI:2052274.1:2000SEP08 | 71742262V1 | 1052 | 1509 |
| 234 | LI:2052274.1:2000SEP08 | 2797262F6 | 1065 | 1642 |
| 234 | LI:2052274.1:2000SEP08 | 2797262H1 | 1065 | 1314 |
| 234 | LI:2052274.1:2000SEP08 | 4157540H1 | 60 | 240 |
| 234 | LI:2052274.1:2000SEP08 | 4157540F8 | 96 559 | |
| 234 | LI:2052274.1:2000SEP08 | 1704531H1 | 160 | 301 |
| 234 | LI:2052274.1:2000SEP08 | 7658244J 1 | 342 | 905 |
| 234 | LI:2052274.1:2000SEP08 | 71741928V1 | 576 | 809 |
| 234 | LI:2052274.1:2000SEP08 | 2831361H1 | 586 | 859 |
| 234 | LI:2052274.1:2000SEP08 | 71744286V1 | 993 | 1509 |
| 234 | LI:2052274.1:2000SEP08 | g1523013 | 960 | 1142 |
| 234 | LI:2052274.1:2000SEP08 | g3678477 | 973 | 1437 |
| 234 | LI:2052274.1:2000SEP08 | 71747321 V1 | 931 | 1123 |
| 234 | LI:2052274.1:2000SEP08 | 71741847V1 | 957 | 1509 |
| 234 | LI:2052274.1:2000SEP08 | 71 743248V1 | 355 | 807 |
| 234 | LI:2052274.1:2000SEP08 | 7986547H1 | 830 | 1316 |
| 234 | LI:2052274.1:2000SEP08 | 71746536V1 | 753 | 1314 |
| 234 | LI:2052274.1:2000SEP08 | 3731660H1 | 750 | 1057 |
| 234 | LI:2052274.1:2000SEP08 | 71745329V1 | 795 | 1125 |
| 234 | LI:2052274.1:2000SEP08 | 71743782V1 | 811 | 1172 |
| 234 | LI:2052274.1:2000SEP08 | 71745857V1 | 811 | 1172 |
| 234 | LI:2052274.1:2000SEP08 | 877185R6 | 341 | 824 |
| 234 | LI:2052274.1:2000SEP08 | 2672580F6 | 559 | 824 |
| 234 | LI:2052274.1:2000SEP08 | 5674936F6 | 355 | 966 |
| 234 | LI:2052274.1:2000SEP08 | 71743956V1 | 355 | 937 |
| 234 | LI:2052274.1:2000SEP08 | 71743912V1 | 355 | 872 |
| 234 | LI:2052274.1:2000SEP08 | 71743192V1 | 351 | 981 |
| 234 | LI:2052274.1:2000SEP08 | 71744831V1 | 355 | 1004 |
| 234 | LI:2052274.1:2000SEP08 | 71742129V1 | 576 | 808 |
| 234 | LI:2052274.1:2000SEP08 | 4333137H1 | 356 | 633 |
| 234 | LI:2052274.1:2000SEP08 | 7762849J1 | 903 | 1561 |
| 234 | LI:2052274.1:2000SEP08 | 2561680H1 | 1452 | 1744 |
| 234 | LI:2052274.1:2000SEP08 | g7456825 | 1461 | 1765 |
| 234 | LI:2052274.1:2000SEP08 | 71744355V1 | 1451 | 1509 |
| 234 | LI:2052274.1:2000SEP08 | 93750627 | 1488 | 1732 |
| 234 | LI:2052274.1:2000SEP08 | g2557941 | 1597 | 1732 |
| 234 | LI:2052274.1:2000SEP08 | 6768646J1 | 1304 | 1524 |
| 234 | LI:2052274.1:2000SEP08 | 71746594V1 | 1336 | 1420 |
| 234 | LI:2052274.1:2000SEP08 | 71742519V1 | 599 | 1286 |
| 234 | LI:2052274.1:2000SEP08 | 6244881H1 | 673 | 752 |
| 234 | LI:2052274.1:2000SEP08 | 71742310V1 | 692 | 1387 |
| 234 | LI:2052274.1:2000SEP08 | 71743501V1 | 748 | 1476 |
| 234 | LI:2052274.1:2000SEP08 | 2672580H1 | 559 | 805 |
| 234 | LI:2052274.1:2000SEP08 | g1523057 | 599 | 1073 |
| 234 | LI:2052274.1:2000SEP08 | 5674936H1 | 355 | 617 |
| 234 | LI:2052274.1:2000SEP08 | 3941090F8 | 8 | 345 |
| 234 | LI:2052274.1:2000SEP08 | 6700546H1 | 1 | 595 |
| 234 | LI:2052274.1:2000SEP08 | 3941090H1 | 8 | 66 |
| 234 | LI:2052274.1:2000SEP08 | 71742091 V1 | 355 | 845 |
| 234 | LI:2052274.1:2000SEP08 | 71743407V1 | 355 | 808 |
| 234 | LI:2052274.1:2000SEP08 | g1950270 | 853 | 1182 |
| 234 | LI:2052274.1:2000SEP08 | 22941 19H1 | 409 | 676 |
| 234 | LI:2052274.1:2000SEP08 | 2113527H1 | 459 | 707 |
| 234 | LI:2052274.1:2000SEP08 | 71 743734V1 | 474 | 980 |
| 234 | LI:2052274.1:2000SEP08 | 3014561H1 | 478 | 776 |
| 234 | LI:2052274.1:2000SEP08 | 5785070H 1 | 554 | 844 |
| 235 | LI:1075502.1:2000SEP08 | 6797669H1 | 1 | 553 |
| 235 | LI:1075502.1:2000SEP08 | 6796045F8 | 1 552 | |
| 235 | LI:1075502.1:2000SEP08 | 6796045T8 | 320 | 924 |
| 235 | LI:1075502.1:2000SEP08 | 6797669T8 | 443 | 858 |
| 235 | LI:1075502.1:2000SEP08 | 6797669F8 | 1 628 | |
| 235 | LI:1075502.1:2000SEP08 | 6796045H1 | 1 | 576 |
| 236 | LI:813697.1:2000SEP08 | 70098909V1 | 962 | 1433 |
| 236 | LI:813697.1:2000SEP08 | 70096440V1 | 963 | 1288 |
| 236 | LI:813697.1:2000SEP08 | 70095040V1 | 1192 | 1603 |
| 236 | LI:813697.1:2000SEP08 | 70098067V1 | 1211 | 1547 |
| 236 | LI:813697.1:2000SEP08 | 620033T6 | 1219 | 1766 |
| 236 | LI:813697.1:2000SEP08 | 598185T6 | 1219 | 1766 |
| 236 | LI:813697.1:2000SEP08 | 6348658F8 | 1244 | 1892 |
| 236 | LI:813697.1:2000SEP08 | 6351670H1 | 1244 | 1566 |
| 236 | LI:813697.1:2000SEP08 | 2373667T6 | 1252 | 1766 |
| 236 | LI:813697.1:2000SEP08 | 70099645V1 | 1260 | 1758 |
| 236 | LI:813697.1:2000SEP08 | g1691544 | 1279 | 1719 |
| 236 | LI:813697.1:2000SEP08 | g5888049 | 1380 | 1800 |
| 236 | LI:813697.1:2000SEP08 | g6838088 | 1415 | 1807 |
| 236 | LI:813697.1:2000SEP08 | g3232965 | 1434 | 1799 |
| 236 | LI:813697.1:2000SEP08 | 5694708H1 | 1462 | 1696 |
| 236 | LI:813697.1:2000SEP08 | g6577365 | 1469 | 1805 |
| 236 | LI:813697.1:2000SEP08 | 70097391V1 | 1483 | 1804 |
| 236 | LI:813697.1:2000SEP08 | 6912262H1 | 1491 | 1980 |
| 236 | LI:813697.1:2000SEP08 | g3145102 | 1537 | 1802 |
| 236 | LI:813697.1:2000SEP08 | 70099964V1 | 1554 | 1804 |
| 236 | LI:813697.1:2000SEP08 | g2695352 | 1676 | 1805 |
| 236 | LI:813697.1:2000SEP08 | 865658R6 | 1703 | 1874 |
| 236 | LI:813697.1:2000SEP08 | 865658H1 | 1703 | 1804 |
| 236 | LI:813697.1:2000SEP08 | 70096773V1 | 636 | 1050 |
| 236 | LI:813697.1:2000SEP08 | 71204835V1 | 597 | 1202 |
| 236 | LI:813697.1:2000SEP08 | 70097075V1 | 892 | 1293 |
| 236 | LI:813697.1:2000SEP08 | 70099616V1 | 899 | 1321 |
| 236 | LI:813697.1:2000SEP08 | 70094872V1 | 579 | 967 |
| 236 | LI:813697.1:2000SEP08 | 70097494V1 | 917 | 1341 |
| 236 | LI:813697.1:2000SEP08 | 71204896V1 | 598 | 1326 |
| 236 | LI:813697.1:2000SEP08 | 7172041H1 | 1 | 529 |
| 236 | LI:813697.1:2000SEP08 | 7280634H1 | 315 | 878 |
| 236 | LI:813697.1:2000SEP08 | 70099218V1 | 389 | 888 |
| 236 | LI:813697.1:2000SEP08 | 70094639V1 | 389 | 758 |
| 236 | LI:813697.1:2000SEP08 | 620033R6 | 389 | 722 |
| 236 | LI:813697.1:2000SEP08 | 620033H1 | 389 | 645 |
| 236 | LI:813697.1:2000SEP08 | 70095662V1 | 411 | 870 |
| 236 | LI:813697.1:2000SEP08 | 1897294H1 | 433 | 681 |
| 236 | LI:813697.1:2000SEP08 | 70099939V1 | 445 | 984 |
| 236 | LI:813697.1:2000SEP08 | 70094912V1 | 511 | 961 |
| 236 | LI:813697.1:20003EP08 | 70099297V1 | 531 | 992 |
| 236 | LI:813697.1:2000SEP08 | 70097017V1 | 539 | 945 |
| 236 | LI:813697.1:2000SEP08 | 70098266V1 | 745 | 1001 |
| 236 | LI:813697.1:2000SEP08 | 70100073V1 | 595 | 1062 |
| 237 | LI:814261.1:2000SEP08 | 959442H1 | 1 | 275 |
| 237 | LI:814261.1:2000SEP08 | 959442R6 | 1 | 366 |
| 237 | LI:814261.1:2000SEP08 | 960385H1 | 1 | 184 |
| 237 | LI:814261.1:2000SEP08 | 338787218 | 185 | 740 |
| 237 | LI:814261.1:2000SEP08 | g2934522 | 342 | 630 |
| 237 | LI:814261.1:2000SEP08 | 6325528H1 | 346 | 538 |
| 237 | LI:814261.1:2000SEP08 | 407022H1 | 371 | 614 |
| 237 | LI:814261.1:2000SEP08 | 960385T6 | 423 | 772 |
| 237 | LI:814261.1:2000SEP08 | g2242138 | 425 | 810 |
| 237 | LI:814261.1:2000SEP08 | 959442T6 | 431 | 761 |
| 237 | LI:814261.1:2000SEP08 | g2969314 | 445 | 541 |
| 238 | LI:775334.1:2000SEP08 | 7331759H1 | 1 | 582 |
| 238 | LI:775334.1:2000SEP08 | 6551320H1 | 191 | 774 |
| 238 | LI:775334.1:2000SEP08 | 6551320F8 | 204 | 726 |
| 238 | LI:775334.1:2000SEP08 | 7331161H1 | 240 | 671 |
| 238 | LI:775334.1:2000SEP08 | g573781 | 414 | 701 |
| 238 | LI:775334.1:2000SEP08 | 3765106H1 | 420 | 716 |
| 238 | LI:775334.1:2000SEP08 | 7403132H1 | 646 | 1274 |
| 238 | LI:775334.1:2000SEP08 | g2219872 | 682 | 1070 |
| 238 | LI:775334.1:2000SEP08 | 6313951H1 | 789 | 1175 |
| 238 | LI:775334.1:2000SEP08 | g775988 | 858 | 1083 |
| 238 | LI:775334.1:2000SEP08 | g1386925 | 917 | 1332 |
| 238 | LI:775334.1:2000SEP08 | g1376602 | 954 | 1380 |
| 238 | LI:775334.1:2000SEP08 | 105380H1 | 1161 | 1369 |
| 238 | LI:775334.1:2000SEP08 | 105380R6 | 1161 | 1486 |
| 238 | LI:775334.1:2000SEP08 | 70932886V1 | 102 | 403 |
| 238 | LI:775334.1:2000SEP08 | 70930434V1 | 173 | 394 |
| 238 | LI:775334.1:2000SEP08 | 70930903V1 | 102 | 404 |
| 238 | LI:775334.1:2000SEP08 | 71276885V1 | 102 | 404 |
| 238 | LI:775334.1:2000SEP08 | 71277249V1 | 173 | 321 |
| 238 | LI:775334.1:2000SEP08 | 70930969V1 | 102 | 420 |
| 238 | LI:775334.1:2000SEP08 | 70931830V1 | 102 | 420 |
| 238 | LI:775334.1:2000SEP08 | 70929143V1 | 173 | 401 |
| 238 | LI:775334.1:2000SEP08 | 70929226V1 | 102 | 420 |
| 238 | LI:775334.1:2000SEP08 | 70929616V1 | 102 | 420 |
| 238 | LI:775334.1:2000SEP08 | 70930408V1 | 102 | 420 |
| 238 | LI:775334.1:2000SEP08 | 70929975V1 | 102 | 420 |
| 238 | LI:775334.1:2000SEP08 | 71278213V1 | 102 | 420 |
| 238 | LI:775334.1:2000SEP08 | 70929941V1 | 102 | 420 |
| 238 | LI:775334.1:2000SEP08 | 7330857H2 | 8 | 383 |
| 238 | LI:775334.1:2000SEP08 | 70931684V1 | 102 | 420 |
| 238 | LI:775334.1:2000SEP08 | 71277794V1 | 102 | 420 |
| 238 | LI:775334.1:2000SEP08 | 71277211V1 | 173 | 284 |
| 238 | LI:775334.1:2000SEP08 | 71277184V1 | 173 | 362 |
| 238 | LI:775334.1:2000SEP08 | 71277083V1 | 102 | 420 |
| 238 | LI:775334.1:2000SEP08 | 71277882V1 | 102 | 420 |
| 238 | LI:775334.1:2000SEP08 | 71278116V1 | 173 | 449 |
| 239 | LI:1180325.1:2000SEP08 | 1438917F6 | 1 | 482 |
| 239 | LI:1180325.1:2000SEP08 | 1438917F1 | 1 | 349 |
| 239 | LI:1180325.1:2000SEP08 | 6102051F8 | 405 | 769 |
| 239 | LI:1180325.1:2000SEP08 | 6102051H1 | 399 | 690 |
| 239 | LI:1180325.1:2000SEP08 | 6102152H1 | 399 | 684 |
| 239 | LI:1180325.1:2000SEP08 | 6102151H1 | 399 | 677 |
| 239 | LI:1180325.1:2000SEP08 | 1438917T6 | 519 | 954 |
| 239 | LI:1180325.1:2000SEP08 | 3138712H1 | 521 | 740 |
| 239 | LI:1180325.1:2000SEP08 | 3138712F6 | 521 | 913 |
| 239 | LI:1180325.1:2000SEP08 | 531270R7 | 555 | 993 |
| 239 | LI:1180325.1:2000SEP08 | 531270R1 | 555 | 993 |
| 239 | LI:1180325.1:2000SEP08 | 531270H1 | 555 | 675 |
| 239 | LI:1180325.1:2000SEP08 | 531270F1 | 555 | 993 |
| 239 | LI:1180325.1:2000SEP08 | 531270T6 | 556 | 956 |
| 239 | LI:1180325.1:2000SEP08 | 4506251H1 | 694 | 968 |
| 239 | LI:1180325.1:2000SEP08 | g1142275 | 837 | 1237 |
| 239 | LI:1180325.1:2000SEP08 | 4203794H1 | 1047 | 1135 |
| 240 | LI:1183147.3:2000SEP08 | 196640R1 | 1 | 438 |
| 240 | LI:1183147.3:2000SEP08 | 196640H1 | 1 | 219 |
| 240 | LI:1183147.3:2000SEP08 | 1360947F6 | 101 | 435 |
| 240 | LI:1183147.3:2000SEP08 | 1360947F1 | 101 | 554 |
| 240 | LI:1183147.3:2000SEP08 | 1360947H1 | 101 | 351 |
| 240 | LI:1183147.3:2000SEP08 | 5015339H1 | 173 | 429 |
| 240 | LI:1183147.3:2000SEP08 | 70651065V1 | 231 | 769 |
| 240 | LI:1183147.3:2000SEP08 | 5174408H1 | 239 | 516 |
| 240 | LI:1183147.3:2000SEP08 | 6516843H1 | 338 | 839 |
| 240 | LI:1183147.3:2000SEP08 | 3751805F6 | 431 | 717 |
| 240 | LI:1183147.3:2000SEP08 | 3751805H1 | 431 | 649 |
| 240 | LI:1183147.3:2000SEP08 | 6941608H1 | 498 | 1015 |
| 240 | LI:1183147.3:2000SEP08 | 5714532H1 | 516 | 806 |
| 240 | LI:1183147.3:2000SEP08 | 70654665V1 | 536 | 1103 |
| 240 | LI:1183147.3:2000SEP08 | 7662413J1 | 542 | 1107 |
| 240 | LI:1183147.3:2000SEP08 | 7090003H1 | 545 | 943 |
| 240 | LI:1183147.3:2000SEP08 | 70653705V1 | 595 | 1197 |
| 240 | LI:1183147.3:2000SEP08 | 70649651 V1 | 707 | 1394 |
| 240 | LI:1183147.3:2000SEP08 | 70651801 V1 | 859 | 1405 |
| 240 | LI:1183147.3:2000SEP08 | 2154418H1 | 820 | 1087 |
| 240 | LI:1183147.3:2000SEP08 | 5743155H1 | 863 | 1144 |
| 240 | LI:1183147.3:2000SEP08 | 6560934T8 | 879 | 1494 |
| 240 | LI:1183147.3:2000SEP08 | 6560934H1 | 880 | 1451 |
| 240 | LI:1183147.3:2000SEP08 | 6560934F8 | 880 | 1603 |
| 240 | LI:1183147.3:2000SEP08 | 2844761H1 | 887 | 1147 |
| 240 | LI:1183147.3:2000SEP08 | 1832094H1 | 935 | 1025 |
| 240 | LI:1183147.3:2000SEP08 | g1300493 | 1073 | 1487 |
| 240 | LI:1183147.3:2000SEP08 | 1007614H1 | 595 | 776 |
| 240 | LI:1183147.3:2000SEP08 | 1401826F6 | 630 | 1115 |
| 240 | LI:1183147.3:2000SEP08 | 1401826H1 | 630 | 876 |
| 240 | LI:1183147.3:2000SEP08 | 1333095F6 | 632 | 949 |
| 240 | LI:1183147.3:2000SEP08 | 70651793V1 | 690 | 1236 |
| 240 | LI:1183147.3:2000SEP08 | 70649650V1 | 706 | 1269 |
| 240 | LI:1183147.3:2000SEP08 | 2597282T6 | 1329 | 1606 |
| 240 | LI:1183147.3:2000SEP08 | g6041091 | 1329 | 1598 |
| 240 | LI:1183147.3:2000SEP08 | g3803626 | 1329 | 1602 |
| 240 | LI:1183147.3:2000SEP08 | g4990175 | 1362 | 1598 |
| 240 | LI:1183147.3:2000SEP08 | g4890819 | 1146 | 1602 |
| 240 | LI:1183147.3:2000SEP08 | 196640R6 | 1 205 | |
| 241 | LI:1175373.3:2000SEP08 | 70394043D1 | 245 | 647 |
| 241 | LI:1175373.3:2000SEP08 | 70503364V1 | 9 | 417 |
| 241 | LI:1175373.3:2000SEP08 | 70392349D1 | 21 | 557 |
| 241 | LI:1175373.3:2000SEP08 | 70393943D1 | 215 | 521 |
| 241 | LI:1175373.3:2000SEP08 | 70503499V1 | 35 722 | |
| 241 | LI:1175373.3:2000SEP08 | 60217417D1 | 39 | 230 |
| 241 | LI:1175373.3:2000SEP08 | 70497402V1 | 281 | 581 |
| 241 | LI:1175373.3:2000SEP08 | 7312966H1 | 317 | 725 |
| 241 | LI:1175373.3:2000SEP08 | 6772905J1 | 1 | 603 |
| 241 | LI:1175373.3:2000SEP08 | 2189954H1 | 9 | 279 |
| 241 | LI:1175373.3:2000SEP08 | 70502455V1 | 9 578 | |
| 241 | LI:1175373.3:2000SEP08 | 2189954F6 | 9 365 | |
| 241 | LI:1175373.3:2000SEP08 | 60217438D1 | 12 | 329 |
| 241 | LI:1175373.3:2000SEP08 | 60217436D1 | 16 | 346 |
| 241 | LI:1175373.3:2000SEP08 | 60217437D1 | 18 | 346 |
| 241 | LI:1175373.3:2000SEP08 | 70355479D1 | 332 | 593 |
| 241 | LI:1175373.3:2000SEP08 | 70393236D1 | 213 | 647 |
| 242 | LI:813757.1:2000SEP08 | 70250012V1 | 7 | 394 |
| 242 | LI:813757.1:2000SEP08 | 70247960V1 | 114 | 615 |
| 242 | LI:813757.1:2000SEP08 | 70248697V1 | 235 | 565 |
| 242 | LI:813757.1:2000SEP08 | 70250218V1 | 161 | 693 |
| 242 | LI:813757.1:2000SEP08 | 70250104V1 | 120 | 722 |
| 242 | LI:813757.1:2000SEP08 | 70250824V1 | 142 | 721 |
| 242 | LI:813757.1:2000SEP08 | 70250254V1 | 221 | 716 |
| 242 | LI:813757.1:2000SEP08 | 70249632V1 | 157 | 707 |
| 242 | LI:813757.1:2000SEP08 | 70247802V1 | 59 | 589 |
| 242 | LI:813757.1:2000SEP08 | 70251286V1 | 1 | 588 |
| 242 | LI:813757.1:2000SEP08 | 3449946R6 | 1 | 577 |
| 242 | LI:813757.1:2000SEP08 | 70251559V1 | 210 | 580 |
| 242 | LI:813757.1:2000SEP08 | 70251049V1 | 1 | 549 |
| 242 | LI:813757.1:2000SEP08 | 70247407V1 | 1 | 535 |
| 242 | LI:813757.1:2000SEP08 | 70251324V1 | 1 | 532 |
| 242 | LI:813757.1:2000SEP08 | 70249664V1 | 1 | 518 |
| 242 | LI:813757.1:2000SEP08 | 70247690V1 | 1 | 506 |
| 242 | LI:813757.1:2000SEP08 | 70247697V1 | 1 | 486 |
| 242 | LI:813757.1:2000SEP08 | 70249740V1 | 2 | 480 |
| 242 | LI:813757.1:2000SEP08 | 70249689V1 | 1 | 265 |
| 242 | LI:813757.1:2000SEP08 | 3A49946H1 | 1 | 234 |
| 242 | LI:813757.1:2000SEP08 | 70258303V1 | 1 | 194 |
| 242 | LI:813757.1:2000SEP08 | 70254826V1 | 1 | 175 |
| 242 | LI:813757.1:2000SEP08 | 3449946T6 | 722 | 1319 |
| 242 | LI:813757.1:2000SEP08 | 70249156V1 | 476 | 965 |
| 242 | LI:813757.1:2000SEP08 | 7289554H1 | 403 | 961 |
| 242 | LI:813757.1:2000SEP08 | 70249867V1 | 464 | 781 |
| 242 | LI:813757.1:2000SEP08 | 70248939V1 | 201 | 768 |
| 242 | LI:813757.1:2000SEP08 | 70251513V1 | 1 | 423 |
| 242 | LI:813757.1:2000SEP08 | 70248335V1 | 80 | 571 |
| 242 | LI:813757.1:2000SEP08 | 70248312V1 | 114 | 647 |
| 242 | LI:813757.1:2000SEP08 | 70250553V1 | 1 | 416 |
| 242 | LI:813757.1:2000SEP08 | 70251476V1 | 83 | 614 |
| 242 | LI:813757.1:2000SEP08 | 7289551H1 | 491 | 961 |
| 242 | LI:813757.1:2000SEP08 | 70248374V1 | 1 | 275 |
| 242 | LI:813757.1:2000SEP08 | 70247985V1 | 196 | 612 |
| 242 | LI:813757.1:2000SEP08 | 70251356V1 | 74 | 581 |
| 242 | LI:813757.1:2000SEP08 | 70250562V1 | 1 | 455 |
| 242 | LI:813757.1:2000SEP08 | 70249004V1 | 79 | 564 |
| 242 | LI:813757.1:2000SEP08 | 70250778V1 | 1 | 446 |
| 242 | LI:813757.1:2000SEP08 | 70251584V1 | 177 | 703 |
| 243 | LI:1182979.2:2000SEP08 | 71191140V1 | 408 | 928 |
| 243 | LI:1182979.2:2000SEP08 | 70739002V1 | 3 | 326 |
| 243 | LI:1182979.2:2000SEP08 | 70735162V1 | 353 | 764 |
| 243 | LI:1182979.2:2000SEP08 | 70733719V1 | 217 | 867 |
| 243 | LI:1182979.2:2000SEP08 | 71191332V1 | 525 | 932 |
| 243 | LI:1182979.2:2000SEP08 | 71188508V1 | 95 | 679 |
| 243 | LI:1182979.2:2000SEP08 | 71188226V1 | 364 | 903 |
| 243 | LI:1182979.2:2000SEP08 | 70738447V1 | 658 | 952 |
| 243 | LI:1182979.2:2000SEP08 | 71131877V1 | 566 | 747 |
| 243 | LI:1182979.2:2000SEP08 | 7073661 1V1 | 3 | 404 |
| 243 | LI:1182979.2:2000SEP08 | 70738052V1 | 125 | 754 |
| 243 | LI:1182979.2:2000SEP08 | 70737437V1 | 458 | 853 |
| 243 | LI:1182979.2:2000SEP08 | 70738236V1 | 373 | 865 |
| 243 | LI:1182979.2:2000SEP08 | 70738437V1 | 658 | 975 |
| 243 | LI:1182979.2:2000SEP08 | 70734169V1 | 366 | 977 |
| 243 | LI:1182979.2:2000SEP08 | 7727943H1 | 310 | 952 |
| 243 | LI:1182979.2:2000SEP08 | 71188062V1 | 502 | 932 |
| 243 | LI:1182979.2:2000SEP08 | 71190218V1 | 324 | 928 |
| 243 | LI:1182979.2:2000SEP08 | 71189394V1 | 491 | 928 |
| 243 | LI:1182979.2:2000SEP08 | 5068784H1 | 617 | 894 |
| 243 | LI:1182979.2:2000SEP08 | 2159096T6 | 797 | 892 |
| 243 | LI:1182979.2:2000SEP08 | 70735390V1 | 451 | 877 |
| 243 | LI:1182979.2:2000SEP08 | 70736220V1 | 362 | 729 |
| 243 | LI:1182979.2:2000SEP08 | 70736517V1 | 43 | 618 |
| 243 | LI:1182979.2:2000SEP08 | 4659839H1 | 1 | 257 |
| 243 | LI:1182979.2:2000SEP08 | 70738465V1 | 144 | 589 |
| 243 | LI:1182979.2:2000SEP08 | 71190329V1 | 410 | 928 |
| 243 | LI:1182979.2:2000SEP08 | 70739216V1 | 382 | 876 |
| 243 | LI:1182979.2:2000SEP08 | 70733762V1 | 359 | 952 |
| 243 | LI:1182979.2:2000SEP08 | 70736375V1 | 412 | 952 |
| 244 | LI:1177823.2:2000SEP08 | 70635125V1 | 738 | 1259 |
| 244 | LI:1177823.2:2000SEP08 | 70637012V1 | 690 | 1259 |
| 244 | LI:1177823.2:2000SEP08 | 70595447V1 | 931 | 1113 |
| 244 | LI:1177823.2:2000SEP08 | 70635133V1 | 642 | 1258 |
| 244 | LI:1177823.2:2000SEP08 | 70639903V1 | 244 | 473 |
| 244 | LI:1177823.2:2000SEP08 | 70634134V1 | 1 | 664 |
| 244 | LI:1177823.2:2000SEP08 | 3042616T6 | 52 | 659 |
| 244 | LI:1177823.2:2000SEP08 | 70641111V1 | 244 | 474 |
| 244 | LI:1177823.2:2000SEP08 | 70635891V1 | 311 | 909 |
| 244 | LI:1177823.2:2000SEP08 | 70635037V1 | 643 | 1270 |
| 244 | LI:1177823.2:2000SEP08 | 70637238V1 | 672 | 1259 |
| 244 | LI:1177823.2:2000SEP08 | 3042616F6 | 685 | 1259 |
| 244 | LI:1177823.2:2000SEP08 | 3055908H1 | 816 | 1102 |
| 244 | LI:1177823.2:2000SEP08 | 3042616H1 | 980 | 1259 |
| 244 | LI:1177823.2:2000SEP08 | 1000943H1 | 1109 | 1358 |
| 244 | LI:1177823.2:2000SEP08 | 2583409H1 | 1295 | 1524 |
| 244 | LI:1177823.2:2000SEP08 | 2583409F6 | 1295 | 1613 |
| 244 | LI:1177823.2:2000SEP08 | 5633561F6 | 1301 | 1892 |
| 244 | LI:1177823.2:2000SEP08 | 5633561H1 | 1301 | 1559 |
| 244 | LI:1177823.2:2000SEP08 | 70637171V1 | 674 | 1259 |
| 244 | LI:1177823.2:2000SEP08 | 70635134V1 | 643 | 1256 |
| 245 | LI:1174279.1:2000SEP08 | 71524916V1 | 332 | 589 |
| 245 | LI:1174279.1:2000SEP08 | 7947644H1 | 135 | 738 |
| 245 | LI:1174279.1:2000SEP08 | g3423045 | 162 | 594 |
| 245 | LI:1174279.1:2000SEP08 | 5997947H1 | 132 | 590 |
| 245 | LI:1174279.1:2000SEP08 | g4222386 | 171 | 592 |
| 245 | LI:1174279.1:2000SEP08 | 60141655V2 | 316 | 610 |
| 245 | LI:1174279.1:2000SEP08 | 505833F1 | 332 | 589 |
| 245 | LI:1174279.1:2000SEP08 | g6576993 | 142 | 589 |
| 245 | LI:1174279.1:2000SEP08 | 71522604V1 | 332 | 589 |
| 245 | LI:1174279.1:2000SEP08 | 60141654V2 | 208 | 607 |
| 245 | LI:1174279.1:2000SEP08 | 6910793J1 | 192 | 804 |
| 245 | LI:1174279.1:2000SEP08 | 7021 1071 V1 | 217 | 589 |
| 245 | LI:1174279.1:2000SEP08 | 70212065V1 | 287 | 589 |
| 245 | LI:1174279.1:2000SEP08 | 505833R6 | 332 | 589 |
| 245 | LI:1174279.1:2000SEP08 | 70212397V1 | 134 | 587 |
| 245 | LI:1174279.1:2000SEP08 | 60141650V2 | 433 | 585 |
| 245 | LI:1174279.1:2000SEP08 | 60141655D2 | 134 | 575 |
| 245 | LI:1174279.1:2000SEP08 | 60141650D1 | 137 | 576 |
| 245 | LI:1174279.1:2000SEP08 | 6013942981 | 134 | 571 |
| 245 | LI:1174279.1:2000SEP08 | 6014165581 | 134 | 567 |
| 245 | LI:1174279.1:2000SEP08 | 6014165481 | 134 | 566 |
| 245 | LI:1174279.1:2000SEP08 | 70212070V1 | 134 | 557 |
| 245 | LI:1174279.1:2000SEP08 | 505833T6 | 332 | 547 |
| 245 | LI:1174279.1:2000SEP08 | 6014270581 | 134 | 534 |
| 245 | LI:1174279.1:2000SEP08 | 505833H1 | 332 | 530 |
| 245 | LI:1174279.1:2000SEP08 | 5947874T8 | 74 473 | |
| 245 | LI:1174279.1:2000SEP08 | 6013943581 | 140 | 489 |
| 245 | LI:1174279.1:2000SEP08 | 5566833H1 | 150 | 374 |
| 245 | LI:1174279.1:2000SEP08 | 4714376H1 | 94 | 353 |
| 245 | LI:1174279.1:2000SEP08 | 5947874H1 | 1 | 259 |
| 245 | LI:1174279.1:2000SEP08 | 7947644J1 | 328 | 901 |
| 246 | LI:1178411.1:2000SEP08 | 71216788V1 | 627 | 1171 |
| 246 | LI:1178411.1:2000SEP08 | 70820046V1 | 627 | 1198 |
| 246 | LI:1178411.1:2000SEP08 | 70821804V1 | 1157 | 1284 |
| 246 | LI:1178411.1:20003EP08 | 70821941V1 | 1155 | 1510 |
| 246 | LI:1178411.1:2000SEP08 | 4002923H1 | 1186 | 1481 |
| 246 | LI:1178411.1:2000SEP08 | 71217149V1 | 1215 | 1474 |
| 246 | LI:1178411.1:2000SEP08 | 5151350H1 | 1415 | 1481 |
| 246 | LI:1178411.1:2000SEP08 | 5151354H1 | 1415 | 1480 |
| 246 | LI:1178411.1:2000SEP08 | 2149114H1 | 1450 | 1510 |
| 246 | LI:1178411.1:2000SEP08 | g6299364 | 1460 | 1533 |
| 246 | LI:1178411.1:2000SEP08 | 2795327H1 | 873 | 1114 |
| 246 | LI:1178411.1:2000SEP08 | 6000894T8 | 925 | 1407 |
| 246 | LI:1178411.1:2000SEP08 | 70819431V1 | 944 | 1502 |
| 246 | LI:1178411.1:2000SEP08 | 70821807V1 | 971 | 1511 |
| 246 | LI:1178411.1:2000SEP08 | 70820608V1 | 985 | 1390 |
| 246 | LI:1178411.1:2000SEP08 | 71216732V1 | 1005 | 1502 |
| 246 | LI:1178411.1:2000SEP08 | 1720617T6 | 1006 | 1463 |
| 246 | LI:1178411.1:2000SEP08 | 71218388V1 | 1025 | 1509 |
| 246 | LI:1178411.1:2000SEP08 | 4030861T6 | 1026 | 1575 |
| 246 | LI:1178411.1:2000SEP08 | 2730285H1 | 1068 | 1299 |
| 246 | LI:1178411.1:2000SEP08 | 1663447H1 | 1071 | 1293 |
| 246 | LI:1178411.1:2000SEP08 | 161226H1 | 1124 | 1349 |
| 246 | LI:1178411.1:2000SEP08 | 70822650V1 | 55 | 608 |
| 246 | LI:1178411.1:2000SEP08 | 70819522V1 | 52 | 501 |
| 246 | LI:1178411.1:2000SEP08 | 70821082V1 | 55 | 645 |
| 246 | LI:1178411.1:2000SEP08 | 70820979V1 | 58 | 659 |
| 246 | LI:1178411.1:2000SEP08 | 5619989H1 | 97 | 373 |
| 246 | LI:1178411.1:2000SEP08 | 71218416V1 | 128 | 770 |
| 246 | LI:1178411.1:2000SEP08 | 71218603V1 | 166 | 746 |
| 246 | LI:1178411.1:2000SEP08 | 70821832V1 | 188 | 615 |
| 246 | LI:1178411.1:2000SEP08 | 71216853V1 | 202 | 749 |
| 246 | LI:1178411.1:2000SEP08 | 1720617H1 | 244 | 438 |
| 246 | LI:1178411.1:2000SEP08 | 1720617F6 | 244 | 686 |
| 246 | LI:1178411.1:2000SEP08 | 70822230V1 | 255 | 700 |
| 246 | LI:1178411.1:2000SEP08 | 70820178V1 | 262 | 776 |
| 246 | LI:1178411.1:2000SEP08 | 70821557V1 | 274 | 913 |
| 246 | LI:1178411.1:2000SEP08 | 70822388V1 | 325 | 934 |
| 246 | LI:1178411.1:2000SEP08 | 71218503V1 | 363 | 772 |
| 246 | LI:1178411.1:2000SEP08 | 5619989R8 | 407 | 617 |
| 246 | LI:1178411.1:2000SEP08 | 4992604F6 | 416 | 975 |
| 246 | LI:1178411.1:2000SEP08 | 4992604H1 | 416 | 621 |
| 246 | LI:1178411.1:2000SEP08 | g1766204 | 442 | 687 |
| 246 | LI:1178411.1:2000SEP08 | 7264011H1 | 460 | 969 |
| 246 | LI:H78411.1:2000SEP08 | 70821108V1 | 460 | 1067 |
| 246 | LI:1178411.1:2000SEP08 | 4856457H1 | 468 | 721 |
| 246 | LI:1178411.1:2000SEP08 | 70821838V1 | 504 | 910 |
| 246 | LI:1178411.1:2000SEP08 | 70822406V1 | 526 | 1106 |
| 246 | LI:1178411.1:2000SEP08 | 70822777V1 | 540 | 888 |
| 246 | LI:1178411.1:2000SEP08 | 70822707V1 | 540 | 886 |
| 246 | LI:1178411.1:2000SEP08 | 70821778V1 | 546 | 1098 |
| 246 | LI:1178411.1:2000SEP08 | 70830924V1 | 570 | 855 |
| 246 | LI:1178411.1:2000SEP08 | 71217684V1 | 582 | 1109 |
| 246 | LI:1178411.1:2000SEP08 | 70820815V1 | 634 | 1154 |
| 246 | LI:1178411.1:2000SEP08 | 70821794V1 | 639 | 855 |
| 246 | LI:1178411.1:2000SEP08 | 71217022V1 | 643 | 1127 |
| 246 | LI:1178411.1:2000SEP08 | 70821968V1 | 671 | 1182 |
| 246 | LI:1178411.1:2000SEP08 | 70819846V1 | 677 | 1274 |
| 246 | LI:1178411.1:2000SEP08 | 70831611V1 | 696 | 1079 |
| 246 | LI:1178411.1:2000SEP08 | 71218453V1 | 718 | 1120 |
| 246 | LI:1178411.1:2000SEP08 | 71218449V1 | 742 | 1154 |
| 246 | LI:1178411.1:2000SEP08 | 70822259V1 | 764 | 1274 |
| 246 | LI:1178411.1:2000SEP08 | 70820312V1 | 781 | 1383 |
| 246 | LI:1178411.1:2000SEP08 | 70821406V1 | 799 | 1352 |
| 246 | LI:1178411.1:2000SEP08 | 4251691H1 | 812 | 1070 |
| 246 | LI:1178411.1:2000SEP08 | 5305837H1 | 824 | 1065 |
| 246 | LI:1178411.1:2000SEP08 | 71217838V1 | 848 | 1220 |
| 246 | LI:1178411.1:2000SEP08 | 71217177V1 | 852 | 1358 |
| 246 | LI:1178411.1:2000SEP08 | 2651913H1 | 852 | 1097 |
| 246 | LI:1178411.1:2000SEP08 | 2651913F6 | 852 | 1399 |
| 246 | LI:1178411.1:2000SEP08 | 4992604T6 | 856 | 1379 |
| 246 | LI:1178411.1:2000SEP08 | 70819531V1 | 859 | 1425 |
| 246 | LI:1178411.1:2000SEP08 | 6000894F8 | 1 427 | |
| 246 | LI:1178411.1:2000SEP08 | 6000894H1 | 1 | 517 |
| 246 | LI:1178411.1:2000SEP08 | 70821182V1 | 53 | 645 |
| 246 | LI:1178411.1:2000SEP08 | 70822648V1 | 52 | 574 |
| 246 | LI:1178411.1:2000SEP08 | 71218324V1 | 52 | 541 |
| 246 | LI:1178411.1:2000SEP08 | 3518577H1 | 52 | 293 |
| 246 | LI:1178411.1:2000SEP08 | 71218530V1 | 52 | 645 |
| 246 | LI:1178411.1:2000SEP08 | 70818807V1 | 52 | 572 |
| 246 | LI:1178411.1:2000SEP08 | 70819287V1 | 52 | 604 |
| 246 | LI:1178411.1:2000SEP08 | 3518577R6 | 52 | 436 |
| 246 | LI:1178411.1:2000SEP08 | 70822421V1 | 52 | 424 |
| 246 | LI:1178411.1:2000SEP08 | 70819408V1 | 52 | 524 |
| 247 | LI:1182739.1:2000SEP08 | 7216759H1 | 1216 | 1364 |
| 247 | LI:1182739.1:2000SEP08 | 3777783H1 | 1493 | 1769 |
| 247 | LI:1182739.1:2000SEP08 | 3204763H1 | 1372 | 1629 |
| 247 | LI:1182739.1:2000SEP08 | g1928274 | 1335 | 1617 |
| 247 | LI:1182739.1:2000SEP08 | g572900 | 1168 | 1521 |
| 247 | LI:1182739.1:2000SEP08 | g1887193 | 965 | 1369 |
| 247 | LI:1182739.1:2000SEP08 | g3751044 | 947 | 1366 |
| 247 | LI:1182739.1:2000SEP08 | 453607T6 | 1949 | 2443 |
| 247 | LI:1182739.1:2000SEP08 | 009464H1 | 2106 | 2354 |
| 247 | LI:1182739.1:2000SEP08 | 3449096H1 | 2149 | 2299 |
| 247 | LI:1182739.1:2000SEP08 | 758641H1 | 2063 | 2289 |
| 247 | LI:1182739.1:2000SEP08 | 4959144F6 | 1508 | 2035 |
| 247 | LI:1182739.1:2000SEP08 | g1275588 | 1535 | 1877 |
| 247 | LI:1182739.1:2000SEP08 | 4959144H1 | 1509 | 1825 |
| 247 | LI:1182739.1:2000SEP08 | 6538604H1 | 1301 | 1802 |
| 247 | LI:1182739.1:2000SEP08 | g6439144 | 901 | 1363 |
| 247 | LI:1182739.1:2000SEP08 | g3899807 | 1069 | 1363 |
| 247 | LI:1182739.1:2000SEP08 | 4361013H1 | 1308 | 1364 |
| 247 | LI:1182739.1:2000SEP08 | g6027047 | 959 | 1360 |
| 247 | LI:1182739.1:2000SEP08 | 71225231V1 | 847 | 1354 |
| 247 | LI:1182739.1:2000SEP08 | 70858587V1 | 760 | 1350 |
| 247 | LI:1182739.1:2000SEP08 | 3559851T6 | 789 | 1327 |
| 247 | LI:1182739.1:2000SEP08 | 70856373V1 | 1104 | 1276 |
| 247 | LI:1182739.1:2000SEP08 | 453607R1 | 631 | 1225 |
| 247 | LI:1182739.1:2000SEP08 | 71226444V1 | 775 | 1217 |
| 247 | LI:1182739.1:2000SEP08 | 195454H1 | 1054 | 1218 |
| 247 | LI:1182739.1:2000SEP08 | g1887192 | 752 | 1214 |
| 247 | LI:1182739.1:2000SEP08 | 71224857V1 | 619 | 1180 |
| 247 | LI:1182739.1:2000SEP08 | 71257619V1 | 459 | 1064 |
| 247 | LI:1182739.1:2000SEP08 | 70856904V1 | 484 | 1063 |
| 247 | LI:1182739.1:2000SEP08 | 71225385V1 | 418 | 1033 |
| 247 | LI:1182739.1:2000SEP08 | 71226469V1 | 482 | 1029 |
| 247 | LI:1182739.1:2000SEP08 | 453607R6 | 631 | 1019 |
| 247 | LI:1182739.1:2000SEP08 | 71226424V1 | 788 | 1002 |
| 247 | LI:1182739.1:2000SEP08 | 70794724V1 | 531 | 980 |
| 247 | LI:1182739.1:2000SEP08 | 71226316V1 | 383 | 915 |
| 247 | LI:1182739.1:2000SEP08 | 71226250V1 | 465 | 908 |
| 247 | LI:1182739.1:2000SEP08 | 71225903V1 | 401 | 896 |
| 247 | LI:1182739.1:2000SEP08 | 453607H1 | 631 | 824 |
| 247 | LI:1182739.1:2000SEP08 | 71225789V1 | 218 | 803 |
| 247 | LI:1182739.1:2000SEP08 | 71225363V1 | 356 | 802 |
| 247 | LI:1182739.1:2000SEP08 | g1230045 | 567 | 750 |
| 247 | LI:1182739.1:2000SEP08 | 71260619V1 | 154 | 715 |
| 247 | LI:1182739.1:2000SEP08 | 71226125V1 | 98 | 693 |
| 247 | LI:1182739.1:2000SEP08 | 71226227V1 | 116 | 689 |
| 247 | LI:1182739.1:2000SEP08 | 8065473J1 | 1 | 669 |
| 247 | LI:1182739.1:2000SEP08 | 70856031V1 | 154 | 661 |
| 247 | LI:1182739.1:2000SEP08 | 70856138V1 | 154 | 474 |
| 247 | LI:1182739.1:2000SEP08 | 6997551H1 | 2225 | 2640 |
| 247 | LI:1182739.1:2000SEP08 | g1231257 | 2295 | 2486 |
| 247 | LI:1182739.1:2000SEP08 | g564488 | 2212 | 2485 |
| 247 | LI:1182739.1:2000SEP08 | 453607F1 | 1886 | 2485 |
| 247 | LI:1182739.1:2000SEP08 | g1928091 | 2175 | 2485 |
| 247 | LI:1182739.1:2000SEP08 | g4334289 | 2055 | 2484 |
| 248 | LI:234937.4:2000SEP08 | 70200118V1 | 198 | 565 |
| 248 | LI:234937.4:2000SEP08 | 6821993J1 | 1 | 276 |
| 248 | LI:234937.4:2000SEP08 | 6821993H1 | 1 | 277 |
| 248 | LI:234937.4:2000SEP08 | 1368116R7 | 27 | 230 |
| 248 | LI:234937.4:2000SEP08 | 1368116H1 | 27 | 178 |
| 248 | LI:234937.4:2000SEP08 | 6201371H1 | 28 | 589 |
| 248 | LI:234937.4:2000SEP08 | 6444066H1 | 86 | 619 |
| 248 | LI:234937.4:2000SEP08 | 70170335V1 | 88 | 512 |
| 248 | LI:234937.4:2000SEP08 | 70199890V1 | 103 | 612 |
| 248 | LI:234937.4:2000SEP08 | 6951283H1 | 119 | 554 |
| 248 | LI:234937.4:2000SEP08 | 5400740H1 | 147 | 415 |
| 248 | LI:234937.4:2000SEP08 | 971204R6 | 164 | 574 |
| 248 | LI:234937.4:2000SEP08 | 971204H7 | 164 | 475 |
| 248 | LI:234937.4:2000SEP08 | 60210282LI1 | 315 | 883 |
| 248 | LI:234937.4:2000SEP08 | 70197633V1 | 381 | 680 |
| 248 | LI:234937.4:2000SEP08 | 70165167V1 | 204 | 493 |
| 248 | LI:234937.4:2000SEP08 | 70166455V1 | 210 | 680 |
| 248 | LI:234937.4:2000SEP08 | 70168325V1 | 207 | 490 |
| 248 | LI:234937.4:2000SEP08 | 5900730H1 | 216 | 482 |
| 248 | LI:234937.4:2000SEP08 | 71704061V1 | 249 | 423 |
| 248 | LI:234937.4:2000SEP08 | 70200163V1 | 268 | 716 |
| 248 | LI:234937.4:2000SEP08 | 70199868V1 | 284 | 711 |
| 248 | LI:234937.4:2000SEP08 | 70165466V1 | 286 | 698 |
| 248 | LI:234937.4:2000SEP08 | 70200075V1 | 297 | 796 |
| 248 | LI:234937.4:2000SEP08 | 70199839V1 | 309 | 674 |
| 248 | LI:234937.4:2000SEP08 | 70199994V1 | 314 | 744 |
| 248 | LI:234937.4:2000SEP08 | 2815595H1 | 313 | 584 |
| 248 | LI:234937.4:2000SEP08 | 971204H1 | 164 | 427 |
| 248 | LI:234937.4:2000SEP08 | 5907378H1 | 177 | 434 |
| 248 | LI:234937.4:2000SEP08 | 70200111V1 | 182 | 708 |
| 248 | LI:234937.4:2000SEP08 | 6439781H1 | 198 | 708 |
| 248 | LI:234937.4:2000SEP08 | 70169923V1 | 386 | 666 |
| 248 | LI:234937.4:2000SEP08 | 70200182V1 | 391 | 835 |
| 248 | LI:234937.4:2000SEP08 | 4245558H1 | 421 | 665 |
| 248 | LI:234937.4:2000SEP08 | 984607H1 | 440 | 753 |
| 248 | LI:234937.4:2000SEP08 | 4194365H1 | 442 | 750 |
| 249 | LI:1170660.1:2000SEP08 | 7582236H1 | 1 | 568 |
| 249 | LI:1170660.1:2000SEP08 | 60218360D1 | 45 | 533 |
| 249 | LI:1170660.1:2000SEP08 | 60218356D1 | 69 | 309 |
| 249 | LI:1170660.1:2000SEP08 | 5547632H1 | 223 | 419 |
| 249 | LI:1170660.1:2000SEP08 | 7186676H1 | 223 | 780 |
| 249 | LI:1170660.1:2000SEP08 | 4706707H1 | 229 | 494 |
| 249 | LI:1170660.1:2000SEP08 | 4543751H1 | 229 | 492 |
| 249 | LI:1170660.1:2000SEP08 | g1635530 | 251 | 552 |
| 249 | LI:1170660.1:2000SEP08 | g1230443 | 257 | 449 |
| 249 | LI:1170660.1:2000SEP08 | 60218359D1 | 264 | 759 |
| 249 | LI:1170660.1:2000SEP08 | 172943H1 | 324 | 547 |
| 249 | LI:1170660.1:2000SEP08 | 60218364D1 | 513 | 923 |
| 249 | LI:1170660.1:2000SEP08 | 4572811H1 | 588 | 716 |
| 249 | LI:1170660.1:2000SEP08 | 7328676H1 | 604 | 1045 |
| 249 | LI:1170660.1:2000SEP08 | g706516 | 643 | 871 |
| 249 | LI:1170660.1:2000SEP08 | g883656 | 644 | 1014 |
| 249 | LI:1170660.1:2000SEP08 | 3806727H1 | 649 | 774 |
| 249 | LI:1170660.1:2000SEP08 | g675102 | 664 | 1020 |
| 249 | LI:1170660.1:2000SEP08 | 60218358D1 | 851 | 1293 |
| 249 | LI:1170660.1:2000SEP08 | 4717311H1 | 889 | 1122 |
| 250 | LI:1144409.1:2000SEP08 | 70858332V1 | 2057 | 2461 |
| 250 | LI:1144409.1:2000SEP08 | 2101604H1 | 2028 | 2294 |
| 250 | LI:1144409.1:2000SEP08 | 2101604R6 | 2028 | 2532 |
| 250 | LI:1144409.1:2000SEP08 | 71225088V1 | 2034 | 2378 |
| 250 | LI:1144409.1:2000SEP08 | g2016400 | 2051 | 2147 |
| 250 | LI:1144409.1:2000SEP08 | 71224917V1 | 2055 | 2615 |
| 250 | LI:1144409.1:2000SEP08 | 804149H1 | 2058 | 2162 |
| 250 | LI:1144409.1:2000SEP08 | 8095372H1 | 1450 | 1744 |
| 250 | LI:1144409.1:2000SEP08 | 3814709H1 | 1477 | 1746 |
| 250 | LI:1144409.1:2000SEP08 | g2809907 | 2239 | 2430 |
| 250 | LI:1144409.1:2000SEP08 | 496959H1 | 2271 | 2449 |
| 250 | LI:1144409.1:2000SEP08 | g1927355 | 2303 | 2606 |
| 250 | LI:1144409.1:2000SEP08 | 70856122V1 | 2062 | 2396 |
| 250 | LI:1144409.1:2000SEP08 | g2540261 | 2063 | 2448 |
| 250 | LI:1144409.1:2000SEP08 | 71227124V1 | 1448 | 1593 |
| 250 | LI:1144409.1:2000SEP08 | 4556204H1 | 1561 | 1642 |
| 250 | LI:1144409.1:2000SEP08 | 4551845F6 | 1561 | 1744 |
| 250 | LI:1144409.1:2000SEP08 | 4265753H1 | 513 | 695 |
| 250 | LI:1144409.1:2000SEP08 | 4337528F6 | 583 | 991 |
| 250 | LI:1144409.1:2000SEP08 | 4337528H1 | 583 | 841 |
| 250 | LI:1144409.1:2000SEP08 | 2792488H1 | 853 | 1139 |
| 250 | LI:1144409.1:2000SEP08 | 2790452H2 | 853 | 1006 |
| 250 | LI:1144409.1:2000SEP08 | 2792488F6 | 853 | 1240 |
| 250 | LI:1144409.1:2000SEP08 | 71225608V1 | 853 | 1382 |
| 250 | LI:1144409.1:2000SEP08 | 8045106H1 | 977 | 1635 |
| 250 | LI:1144409.1:2000SEP08 | 7269729H1 | 1056 | 1608 |
| 250 | LI:1144409.1:2000SEP08 | 7617315H1 | 1 | 258 |
| 250 | LI:1144409.1:2000SEP08 | 4841542H1 | 12 | 274 |
| 250 | LI:1144409.1:2000SEP08 | 7609838J1 | 42 | 593 |
| 250 | LI:1144409.1:2000SEP08 | 7617315J1 | 307 | 888 |
| 250 | LI:1144409.1:2000SEP08 | 76541 74H1 | 356 | 465 |
| 250 | LI:1144409.1:2000SEP08 | 7697207H1 | 399 | 860 |
| 250 | LI:1144409.1:2000SEP08 | 7697207J1 | 399 | 886 |
| 250 | LI:1144409.1:2000SEP08 | 7608868J1 | 407 | 762 |
| 250 | LI:1144409.1:2000SEP08 | 8045106J1 | 427 | 867 |
| 250 | LI:1144409.1:2000SEP08 | 630611H1 | 2382 | 2607 |
| 250 | LI:1144409.1:2000SEP08 | 904646R2 | 2397 | 2601 |
| 250 | LI:1144409.1:2000SEP08 | 904646H1 | 2398 | 2608 |
| 250 | LI:1144409.1:2000SEP08 | 2130512H1 | 2412 | 2606 |
| 250 | LI:1144409.1:2000SEP08 | 4383061H1 | 2412 | 2583 |
| 250 | LI:1144409.1:2000SEP08 | g5632630 | 2501 | 2607 |
| 250 | LI:1144409.1:2000SEP08 | 406063H1 | 2316 | 2543 |
| 250 | LI:1144409.1:2000SEP08 | 7335618H1 | 2331 | 2616 |
| 250 | LI:1144409.1:2000SEP08 | g4329276 | 2347 | 2613 |
| 250 | LI:1144409.1:2000SEP08 | 3219963H1 | 2062 | 2228 |
| 250 | LI:1144409.1:2000SEP08 | 3485587H1 | 2062 | 2165 |
| 250 | LI:1144409.1:2000SEP08 | 6020943H 1 | 2062 | 2430 |
| 250 | LI:1144409.1:2000SEP08 | 70857496V1 | 1351 | 1744 |
| 250 | LI:1144409.1:2000SEP08 | 8093771H1 | 1426 | 1744 |
| 250 | LI:1144409.1:2000SEP08 | 505559H1 | 2150 | 2362 |
| 250 | LI:1144409.1:2000SEP08 | 2792488T6 | 2157 | 2565 |
| 250 | LI:1144409.1:2000SEP08 | 632101H1 | 2161 | 2424 |
| 250 | LI:1144409.1:2000SEP08 | 3479107H1 | 2175 | 2356 |
| 250 | LI:1144409.1:2000SEP08 | 822341R1 | 2189 | 2449 |
| 250 | LI:1144409.1:2000SEP08 | 822341H1 | 2189 | 2412 |
| 250 | LI:1144409.1:2000SEP08 | 70388384D1 | 2198 | 2607 |
| 250 | LI:1144409.1:2000SEP08 | g3086258 | 2215 | 2609 |
| 250 | LI:1144409.1:2000SEP08 | 2101604T6 | 2217 | 2565 |
| 250 | LI:1144409.1:2000SEP08 | 636591H1 | 2217 | 2470 |
| 250 | LI:1144409.1:2000SEP08 | g3961700 | 2503 | 2607 |
| 250 | LI:1144409.1:2000SEP08 | 71226556V1 | 1132 | 1744 |
| 250 | LI:1144409.1:2000SEP08 | 71225104V1 | 1161 | 1744 |
| 250 | LI:1144409.1:2000SEP08 | g1802842 | 1156 | 1515 |
| 250 | LI:1144409.1:2000SEP08 | 71226355V1 | 1220 | 1744 |
| 250 | LI:1144409.1:2000SEP08 | 5734055H1 | 1230 | 1497 |
| 250 | LI:1144409.1:2000SEP08 | 71225770V1 | 1232 | 1744 |
| 250 | LI:1144409.1:2000SEP08 | 70855263V1 | 1247 | 1744 |
| 250 | LI:1144409.1:2000SEP08 | 70858102V1 | 1275 | 1762 |
| 250 | LI:1144409.1:2000SEP08 | 6870517H1 | 1296 | 1814 |
| 250 | LI:1144409.1:2000SEP08 | 71225590V1 | 1349 | 1744 |
| 250 | LI:1144409.1:2000SEP08 | g2011857 | 2103 | 2393 |
| 250 | LI:1144409.1:2000SEP08 | 2875837H1 | 2116 | 2394 |
| 250 | LI:1144409.1:2000SEP08 | 71226547V1 | 2122 | 2618 |
| 250 | LI:1144409.1:20003EP08 | g1849425 | 2131 | 2424 |
| 250 | LI:1144409.1:2000SEP08 | 4337528T6 | 2144 | 2581 |
| 250 | LI:1144409.1:2000SEP08 | 3118103F6 | 2146 | 2582 |
| 250 | LI:1144409.1:2000SEP08 | 3118103H1 | 2147 | 2437 |
| 250 | LI:1144409.1:2000SEP08 | 3814709F7 | 1498 | 1730 |
| 250 | LI:1144409.1:2000SEP08 | 70854990V1 | 1522 | 1744 |
| 250 | LI:1144409.1:2000SEP08 | 4557749H1 | 1561 | 1744 |
| 250 | LI:1144409.1:2000SEP08 | 4556204F8 | 1561 | 2151 |
| 250 | LI:1144409.1:2000SEP08 | 4557749F8 | 1580 | 2139 |
| 250 | LI:1144409.1:2000SEP08 | 70856541V1 | 1580 | 1766 |
| 250 | LI:1144409.1:2000SEP08 | 70856732V1 | 1601 | 1744 |
| 250 | LI:1144409.1:2000SEP08 | 70858292V1 | 1605 | 2254 |
| 250 | LI:1144409.1:2000SEP08 | 70856003V1 | 1634 | 2192 |
| 250 | LI:1144409.1:2000SEP08 | 70857442V1 | 1649 | 1744 |
| 250 | LI:1144409.1:2000SEP08 | 8038711H1 | 1794 | 2407 |
| 250 | LI:1144409.1:2000SEP08 | 3999943H1 | 2062 | 2229 |
| 250 | LI:1144409.1:2000SEP08 | 6147820H1 | 2078 | 2363 |
| 250 | LI:1144409.1:2000SEP08 | 71225849V1 | 2080 | 2376 |
| 250 | LI:1144409.1:2000SEP08 | 71225565V1 | 2080 | 2316 |
| 250 | LI:1144409.1:2000SEP08 | 4445313H1 | 2080 | 2266 |
| 250 | LI:1144409.1:2000SEP08 | 955100T2 | 2090 | 2570 |
| 250 | LI:1144409.1:2000SEP08 | 955100R1 | 2090 | 2453 |
| 250 | LI:1144409.1:2000SEP08 | 955100H1 | 2090 | 2358 |
| 250 | LI:1144409.1:2000SEP08 | 3814175T6 | 2095 | 2570 |
| 251 | LI:246290.10:2000SEP08 | 7160791H1 | 485 | 997 |
| 251 | LI:246290.10:2000SEP08 | 1320454H1 | 499 | 734 |
| 251 | LI:246290.10:2000SEP08 | 7644139J1 | 512 | 1059 |
| 251 | LI:246290.10:2000SEP08 | 6723259H1 | 516 | 916 |
| 251 | LI:246290.10:2000SEP08 | 747973R6 | 330 | 859 |
| 251 | LI:246290.10:2000SEP08 | 747973H1 | 330 | 572 |
| 251 | LI:246290.10:2000SEP08 | 7170570H1 | 383 | 910 |
| 251 | LI:246290.10:2000SEP08 | 5080008H1 | 88 | 328 |
| 251 | LI:246290.10:2000SEP08 | 5957953H1 | 89 | 545 |
| 251 | LI:246290.10:2000SEP08 | 6937991H1 | 99 | 200 |
| 251 | LI:246290.10:2000SEP08 | 7168155H1 | 137 | 243 |
| 251 | LI:246290.10:2000SEP08 | 4831526F8 | 229 | 767 |
| 251 | LI:246290.10:2000SEP08 | 4831526H1 | 230 | 359 |
| 251 | LI:246290.10:2000SEP08 | 4831526F9 | 229 | 744 |
| 251 | LI:246290.10:2000SEP08 | 7387420H1 | 285 | 504 |
| 251 | LI:246290.10:2000SEP08 | 5761576H1 | 540 | 807 |
| 251 | LI:246290.10:2000SEP08 | 4930979H1 | 612 | 891 |
| 251 | LI:246290.10:2000SEP08 | 806868H1 | 728 | 958 |
| 251 | LI:246290.10:2000SEP08 | 6866060H1 | 68 | 403 |
| 251 | LI:246290.10:2000SEP08 | 3506257H1 | 73 | 350 |
| 251 | LI:246290.10:2000SEP08 | 3550395H1 | 1 | 288 |
| 251 | LI:246290.10:2000SEP08 | 7988114H1 | 2 | 511 |
| 251 | LI:246290.10:2000SEP08 | 3316757H1 | 18 | 304 |
| 251 | LI:246290.10:2000SEP08 | 8122994H1 | 19 | 612 |
| 251 | LI:246290.10:2000SEP08 | 5457648H1 | 23 | 233 |
| 251 | LI:246290.10:2000SEP08 | 3508411H1 | 23 | 303 |
| 251 | LI:246290.10:2000SEP08 | 3577541H1 | 23 | 287 |
| 251 | LI:246290.10:2000SEP08 | 5460535H1 | 23 | 271 |
| 251 | LI:246290.10:2000SEP08 | 7002420H1 | 39 | 596 |
| 251 | LI:246290.10:2000SEP08 | 4328953H1 | 42 | 304 |
| 251 | LI:246290.10:2000SEP08 | 3973335H1 | 57 | 325 |
| 251 | LI:246290.10:2000SEP08 | 7470753H1 | 67 | 633 |
| 251 | LI:246290.10:2000SEP08 | 2966230H1 | 1 | 297 |
| 251 | LI:246290.10:2000SEP08 | 2966230F6 | 1 | 377 |
| 252 | LI:280034.1:2000SEP08 | 8045415H1 | 1 | 646 |
| 252 | LI:280034.1:2000SEP08 | 8045415J1 | 482 | 1096 |
| 252 | LI:280034.1:2000SEP08 | 681866T6 | 584 | 802 |
| 252 | LI:280034.1:2000SEP08 | 681866H1 | 584 | 845 |
| 252 | LI:280034.1:2000SEP08 | 681866R6 | 584 | 841 |
| 252 | LI:280034.1:2000SEP08 | 4936924H1 | 781 | 1020 |

TABLE 6

| SEQ ID NO: | Template ID | Tissue Distribution |
|---|---|---|
| 1 | LG:150318.1:2000SEP08 | Nervous System - 100% |
| 2 | LG:022529.1:2000SEP08 | Musculoskeletal System - 25%, Unclassified/Mixed - 22%, Cardiovascular |
| | | System - 14% |
| 3 | LG:352559.1:2000SEP08 | Unclassified/Mixed - 67%, Digestive System - 33% |
| 4 | LG:175223.1:2000SEP08 | Embryonic Structures - 82%, Nervous System - 18% |
| 5 | LG:476989.1:2000SEP08 | Exocrine Glands - 62%, Urinary Tract - 31% |
| 6 | LG:253268.7:2000SEP08 | Germ Cells - 56%, Nervous System - 23% |
| 7 | LG:401322.1:2000SEP08 | Sense Organs - 50%, Liver - 17%, Skin - 13% |
| 8 | LG:1328436.1:2000SEP08 | Respiratory System - 38%, Endocrine System - 35%, Connective Tissue - |
| | | 27% |
| 9 | LG:475404.1:2000SEP08 | Skin - 81% |
| 10 | LG:1384132.1:2000SEP08 | Respiratory System - 50%, Digestive System - 33%, Nervous System - 17% |
| 11 | LG:410804.18:2000SEP08 | Nervous System - 100% |
| 12 | LG:1082306.1:2000SEP08 | Cardiovascular System - 28%, Digestive System - 21%, Exocrine Glands - |
| | | 14%, Endocrine System - 14% |
| 13 | LG:233814.4:2000SEP08 | Endocrine System - 36%, Pancreas - 36%, Exocrine Glands - 16% |
| 14 | LG:977478.5:2000SEP08 | Cardiovascular System - 34%, Musculoskeletal System - 19%, Female |
| | | Genitalia - 16% |
| 15 | LG:025931.1:2000SEP08 | Female Genitalia - 29%, Urinary Tract - 24%, Endocrine System - 24% |
| 16 | LG:885368.1:2000SEP08 | Nervous System - 67%, Female Genitalia - 33% |
| 17 | LG:1054900.1:2000SEP08 | Digestive System - 50%, Female Genitalia - 25%, Male Genitalia - 25% |
| 18 | LG:995186.2:2000SEP08 | Digestive System - 67%, Nervous System - 33% |
| 19 | LG:435048.23:2000SEP08 | Urinary Tract - 80%, Nervous System - 20% |
| 20 | LG:954859.1:2000SEP08 | Embryonic Structures - 56%, Hemic and Immune System - 19%, Female |
| | | Genitalia - 13%, Nervous System - 13% |
| 21 | LG:364370.1:2000SEP08 | Liver - 90%, Male Genitalia - 10% |
| 22 | LG:1098789.1:2000SEP08 | Urinary Tract - 100% |
| 23 | LG:201540.2:2000SEP08 | Unclassified/Mixed - 15%, Female Genitalia - 13%, Connective Tissue - |
| | | 13%, Male Genitalia - 13% |
| 24 | LG:1077357.1:2000SEP08 | Nervous System - 43%, Male Genitalia - 29%, Female Genitalia - 29% |
| 25 | LG:1048846.4:2000SEP08 | Female Genitalia - 25%, Digestive System - 25%, Male Genitalia - 25% |
| 26 | LG:336685.1:2000SEP08 | Hemic and Immune System - 35%, Urinary Tract - 24%, Male Genitalia - |
| | | 24% |
| 27 | LG:1076253.1:2000SEP08 | Liver - 50%, Urinary Tract - 11%, Endocrine System - 11% |
| 28 | LG:1400601.2:2000SEP08 | Skin - 100% |
| 29 | LG:1079092.3:2000SEP08 | Musculoskeletal System - 100% |
| 30 | LG:1086064.1:2000SEP08 | Skin - 69% |
| 31 | LG:1400608.1:2000SEP08 | Female Genitalia - 26%, Urinary Tract - 21%, Respiratory System - 16% |
| 32 | LG:399275.5:2000SEP08 | Respiratory System - 50%, Male Genitalia - 33%, Hemic and Immune |
| | | System - 17% |
| 33 | LG:293943.1:2000SEP08 | Exocrine Glands - 50%, Digestive System - 25%, Hemic and Immune |
| | | System - 13%, Nervous System - 13% |
| 34 | LG:345884.1:2000SEP08 | Respiratory System - 63%, Hemic and Immune System - 38% |
| 35 | LG:400967.1:2000SEP08 | Embryonic Structures - 36%, Urinary Tract - 28%, Cardiovascular System - |
| | | 16% |
| 36 | LG:024556.6:2000SEP08 | Nervous System - 89%, Male Genitalia - 11% |
| 37 | LG:081189.3:2000SEP08 | Germ Cells - 88% |
| 38 | LG:018258.1:2000SEP08 | Digestive System - 44%, Endocrine System - 44%, Hemic and Immune |
| | | System - 11% |
| 39 | LG:450399.3:2000SEP08 | Nervous System - 100% |
| 40 | LG:451122.1:2000SEP08 | Nervous System - 100% |
| 41 | LG:451682.1:2000SEP08 | Nervous System - 100% |
| 42 | LG:238631.4:2000SEP08 | Liver - 17%, Endocrine System - 14%, Respiratory System - 11%, Male |
| | | Genitalia - 11% |
| 43 | LG:236654.1:2000SEP08 | Unclassified/Mixed - 38%, Respiratory System - 15% |
| 44 | LG:332655.1:2000SEP08 | Pancreas - 26%, Digestive System - 17%, Female Genitalia - 13% |
| 45 | LG:217396.2:2000SEP08 | Skin - 24%, Embryonic Structures - 15%, Pancreas - 15% |
| 46 | LG:090574.1:2000SEP08 | Respiratory System - 100% |
| 47 | LG:202943.1:2000SEP08 | Embryonic Structures - 44%, Female Genitalia - 19%, Liver - 14% |
| 48 | LG:236928.1:2000SEP08 | Unclassified/Mixed - 15%, Hemic and Immune System - 14%, Nervous |
| | | System - 12% |
| 49 | LG:215169.2:2000SEP08 | Endocrine System - 22%, Unclassified/Mixed - 20%, Nervous System - 20% |
| 50 | LG:410726.1:2000SEP08 | Embryonic Structures - 38%, Pancreas - 24%, Endocrine System - 18% |
| 51 | LG:234372.2:2000SEP08 | Stomatognathic System - 17%, Germ Cells - 12%, Musculoskeletal System - |
| | | 11% |
| 52 | LG:022629.1:2000SEP08 | Unclassified/Mixed - 36%, Germ Cells - 28% |
| 53 | LG:068682.1:2000SEP08 | Unclassified/Mixed - 61%, Male Genitalia - 20% |
| 54 | LG:222335.1:2000SEP08 | Musculoskeletal System - 27%, Exocrine Glands - 17%, Male Genitalia - |
| | | 13% |
| 55 | LG:331342.1:2000SEP08 | Male Genitalia - 35%, Digestive System - 22%, Endocrine System - 20% |
| 56 | LG:021770.1:2000SEP08 | Pancreas - 18%, Exocrine Glands - 14%, Nervous System - 12%, Urinary |
| | | Tract - 12%, Male Genitalia - 12% |
| 57 | LG:181607.9:2000SEP08 | Musculoskeletal System - 25%, Cardiovascular System - 22%, Embryonic |
| | | Structures - 18% |
| 58 | LG:1042768.1:2000SEP08 | Liver - 100% |
| 59 | LG:282729.1:2000SEP08 | Skin - 74%, Unclassified/Mixed - 21% |
| 60 | LG:998305.3:2000SEP08 | Urinary Tract - 50%, Cardiovascular System - 29%, Nervous System - 21% |
| 61 | LG:1135213.1:2000SEP08 | Embryonic Structures - 26%, Cardiovascular System - 20%, Digestive |
| | | System - 11%, Unclassified/Mixed - 11% |
| 62 | LG:267762.1:2000SEP08 | Cardiovascular System - 16%, Urinary Tract - 14%, Connective Tissue - |
| | | 14% |
| 63 | LG:120744.1:2000SEP08 | Skin - 35%, Embryonic Structures - 23%, Digestive System - 20% |
| 64 | LG:403409.1:2000SEP08 | Stomatognathic System - 45%, Respiratory System - 12% |
| 65 | LG:226874.3:2000SEP08 | Respiratory System - 38%, Male Genitalia - 35%, Female Genitalia - 19% |
| 66 | LG:1045521.4:2000SEP08 | Germ Cells - 16% |
| 67 | LG:275876.1:2000SEP08 | Unclassified/Mixed - 53%, Endocrine System - 27%, Nervous System - 20% |
| 68 | LG:475127.7:2000SEP08 | Hemic and Immune System - 75%, Nervous System - 25% |
| 69 | LG:157263.1:2000SEP08 | Embryonic Structures - 39%, Urinary Tract - 30% |
| 70 | LG:247382.7:2000SEP08 | Urinary Tract - 19%, Hemic and Immune System - 13%, Embryonic |
| | | Structures - 13%, Endocrine System - 13% |
| 71 | LG:197367.5:2000SEP08 | Endocrine System - 100% |
| 72 | LG:218090.5:2000SEP08 | Unclassified/Mixed - 42%, Urinary Tract - 21%, Exocrine Glands - 21% |
| 73 | LG:216612.4:2000SEP08 | Nervous System - 45%, Digestive System - 30%, Endocrine System - 20% |
| 74 | LG:197614.1:2000SEP08 | Germ Cells - 17%, Unclassified/Mixed - 16% |
| 75 | LG:378428.1:2000SEP08 | Liver - 30%, Endocrine System - 15%, Embryonic Structures - 15% |
| 76 | LG:286639.1:2000SEP08 | Germ Cells - 72% |
| 77 | LG:389870.1:2000SEP08 | Liver - 41%, Nervous System - 23%, Cardiovascular System - 18%, |
| | | Endocrine System - 18% |
| 78 | LG:1387485.6:2000SEP08 | Female Genitalia - 16%, Digestive System - 14%, Urinary Tract - 13% |
| 79 | LG:230151.1:2000SEP08 | Exocrine Glands - 16%, Urinary Tract - 14%, Liver - 12%, Pancreas - 12% |
| 80 | LG:215158.5:2000SEP08 | Liver - 13%, Nervous System - 12%, Unclassified/Mixed - 11% |
| 81 | LG:235840.1:2000SEP08 | Connective Tissue - 13%, Nervous System - 12%, Hemic and Immune |
| | | System - 11%, Liver - 11% |
| 82 | LG:350272.1:2000SEP08 | Germ Cells - 17%, Musculoskeletal System - 15% |
| 83 | LG:232190.1:2000SEP08 | Liver - 24%, Unclassified/Mixed - 23%, Skin - 19% |
| 84 | LG:1068127.1:2000SEP08 | Hemic and Immune System - 43%, Male Genitalia - 29%, Nervous System - |
| | | 29% |
| 85 | LG:408751.3:2000SEP08 | Nervous System - 43%, Sense Organs - 31% |
| 86 | LG:1078933.1:2000SEP08 | Liver - 23%, Unclassified/Mixed - 20%, Nervous System - 13% |
| 87 | LG:958731.1:2000SEP08 | Nervous System - 100% |
| 88 | LG:024125.5:2000SEP08 | Connective Tissue - 17%, Embryonic Structures - 16% |
| 89 | LG:373637.3:2000SEP08 | Unclassified/Mixed - 73%, Male Genitalia - 23% |
| 90 | LG:1053229.1:2000SEP08 | Cardiovascular System - 50%, Embryonic Structures - 41% |
| 91 | LG:248364.1:2000SEP08 | Sense Organs - 48%, Female Genitalia - 17% |
| 92 | LG:477130.1:2000SEP08 | Nervous System - 100% |
| 93 | LG:113786.17:2000SEP08 | Hemic and Immune System - 100% |
| 94 | LG:347635.1:2000SEP08 | Musculoskeletal System - 45%, Female Genitalia - 17%, Urinary Tract - |
| | | 14% |
| 95 | LG:242966.4:2000SEP08 | Germ Cells - 23%, Stomatognathic System - 23% |
| 96 | LG:217814.1:2000SEP08 | Skin - 25%, Liver - 16%, Unclassified/Mixed - 15% |
| 97 | LG:476452.1:2000SEP08 | Nervous System - 100% |
| 98 | LG:1100657.1:2000SEP08 | Liver - 100% |
| 99 | LG:1132418.2:2000SEP08 | Cardiovascular System - 100% |
| 100 | LG:1098570.1:2000SEP08 | Liver - 100% |
| 101 | LG:1097987.1:2000SEP08 | Embryonic Structures - 41%, Musculoskeletal System - 27%, Female |
| | | Genitalia - 23% |
| 102 | LG:337818.2:2000SEP08 | Digestive System - 35%, Liver - 13%, Female Genitalia - 10% |
| 103 | LG:1040582.1:2000SEP08 | Liver - 38%, Pancreas - 38%, Cardiovascular System - 17% |
| 104 | LG:1099122.1:2000SEP08 | Liver - 96% |
| 105 | LG:1327449.1:2000SEP08 | Endocrine System - 40%, Respiratory System - 30%, Digestive System - |
| | | 20% |
| 106 | LG:227933.5:2000SEP08 | Male Genitalia - 33%, Nervous System - 21%, Female Genitalia - 15% |
| 107 | LG:1043709.2:2000SEP08 | Liver - 100% |
| 108 | LG:1099871.1:2000SEP08 | Liver - 90%, Nervous System - 10% |
| 109 | LG:1399139.4:2000SEP08 | Unclassified/Mixed - 30%, Male Genitalia - 26%, Musculoskeletal System - |
| | | 22% |
| 110 | LG:236386.1:2000SEP08 | Skin - 16%, Connective Tissue - 13%, Pancreas - 10% |
| 111 | LG:1015157.1:2000SEP08 | Liver - 100% |
| 112 | LG:1065433.1:2000SEP08 | Female Genitalia - 50%, Endocrine System - 40%, Nervous System - 10% |
| 113 | LG:236992.4:2000SEP08 | Connective Tissue - 64%, Urinary Tract - 12%, Cardiovascular System - |
| | | 12% |
| 114 | LG:1071124.1:2000SEP08 | Unclassified/Mixed - 22%, Cardiovascular System - 22%, Urinary Tract - |
| | | 19% |
| 115 | LG:206425.2:2000SEP08 | Sense Organs - 39%, Female Genitalia - 10% |
| 116 | LG:885747.2:2000SEP08 | Female Genitalia - 100% |
| 117 | LG:1140501.1:2000SEP08 | Sense Organs - 20%, Nervous System - 15% |
| 118 | LG:001239.1:2000SEP08 | Hemic and Immune System - 35%, Liver - 17%, Male Genitalia - 13% |
| 119 | LG:018980.1:2000SEP08 | Unclassified/Mixed - 52%, Urinary Tract - 12%, Nervous System - 12% |
| 120 | LG:1083120.3:2000SEP08 | Digestive System - 50%, Hemic and Immune System - 25%, Nervous |
| | | System - 25% |
| 121 | LG:233258.3:2000SEP08 | Germ Cells - 25%, Female Genitalia - 12% |
| 122 | LG:999062.1:2000SEP08 | Nervous System - 100% |
| 123 | LG:887776.1:2000SEP08 | Cardiovascular System - 35%, Skin - 33%, Hemic and Immune System - |
| | | 14% |
| 124 | LG:1400301.2:2000SEP08 | Exocrine Glands - 50%, Connective Tissue - 44% |
| 125 | LG:1329362.1:2000SEP08 | Unclassified/Mixed - 44%, Urinary Tract - 22%, Exocrine Glands - 22% |
| 126 | LG:1096498.1:2000SEP08 | Liver - 100% |
| 127 | LG:1096337.1:2000SEP08 | Urinary Tract - 40%, Exocrine Glands - 40%, Nervous System - 20% |
| 128 | LG:1400579.1:2000SEP08 | Liver - 53%, Musculoskeletal System - 25% |
| 129 | LG:1080091.1:2000SEP08 | Connective Tissue - 21%, Musculoskeletal System - 18%, Female |
| | | Genitalia - 15% |
| 130 | LG:1082203.1:2000SEP08 | Embryonic Structures - 23%, Endocrine System - 11%, Male Genitalia - |
| | | 11% |
| 131 | LG:1084051.1:2000SEP08 | Germ Cells - 33%, Pancreas - 11%, Male Genitalia - 11% |
| 132 | LG:1082393.1:2000SEP08 | Male Genitalia - 20%, Urinary Tract - 13%, Unclassified/Mixed - 13% |
| 133 | LG:1086183.1:2000SEP08 | Liver - 25%, Skin - 20%, Hemic and Immune System - 14% |
| 134 | LG:1090268.1:2000SEP08 | Sense Organs - 40%, Unclassified/Mixed - 17% |
| 135 | LG:1400597.5:2000SEP08 | Unclassified/Mixed - 53%, Connective Tissue - 47% |
| 136 | LG:1080307.2:2000SEP08 | Digestive System - 100% |
| 137 | LG:1400603.2:2000SEP08 | Germ Cells - 40%, Nervous System - 18%, Unclassified/Mixed - 12% |
| 138 | LG:1052984.1:2000SEP08 | Skin - 31%, Musculoskeletal System - 15%, Endocrine System - 10%, |
| | | Pancreas - 10% |
| 139 | LG:1091259.1:2000SEP08 | Connective Tissue - 66%, Respiratory System - 25% |
| 140 | LG:1082263.2:2000SEP08 | Embryonic Structures - 32%, Endocrine System - 15%, Nervous System - |
| | | 10% |
| 141 | LG:1048604.2:2000SEP08 | Exocrine Glands - 50%, Embryonic Structures - 38% |
| 142 | LG:1085254.3:2000SEP08 | Embryonic Structures - 61%, Respiratory System - 32% |
| 143 | LG:1400606.2:2000SEP08 | Embryonic Structures - 34%, Unclassified/Mixed - 14%, Nervous System - |
| | | 13%, Connective Tissue - 13% |
| 144 | LG:1090358.2:2000SEP08 | Urinary Tract - 36%, Male Genitalia - 23%, Hemic and Immune System - |
| | | 15%, Musculoskeletal System - 15% |
| 145 | LG:1079064.2:2000SEP08 | Nervous System - 21%, Female Genitalia - 16%, Liver - 12% |
| 146 | LG:1076866.1:2000SEP08 | Germ Cells - 28%, Embryonic Structures - 20%, Cardiovascular System - |
| | | 12% |
| 147 | LG:969359.1:2000SEP08 | Liver - 79%, Pancreas - 19% |
| 148 | LG:366783.1:2000SEP08 | Sense Organs - 95% |
| 149 | LG:332176.3:2000SEP08 | Pancreas - 28%, Connective Tissue - 22%, Urinary Tract - 22% |
| 150 | LG:994938.1:2000SEP08 | Exocrine Glands - 50%, Respiratory System - 21%, Urinary Tract - 17% |
| 151 | LG:982800.1:2000SEP08 | Urinary Tract - 22%, Skin - 21%, Germ Cells - 13% |
| 152 | LG:977850.7:2000SEP08 | Exocrine Glands - 100% |
| 153 | LG:234748.2:2000SEP08 | Sense Organs - 27%, Urinary Tract - 15%, Unclassified/Mixed - 11% |
| 154 | LG:306284.1:2000SEP08 | Nervous System - 60%, Male Genitalia - 40% |
| 155 | LI:333170.3:2000SEP08 | Germ Cells - 82%, Unclassified/Mixed - 13% |
| 156 | LI:336685.2:2000SEP08 | Hemic and Immune System - 35%, Male Genitalia - 25%, Urinary Tract - |
| | | 20% |
| 157 | LI:279013.5:2000SEP08 | Female Genitalia - 100% |
| 158 | LI:1037075.1:2000SEP08 | Musculoskeletal System - 94% |
| 159 | LI:1073403.1:2000SEP08 | Liver - 100% |
| 160 | LI:1075296.1:2000SEP08 | Liver - 100% |
| 161 | LI:1085501.1:2000SEP08 | Connective Tissue - 100% |
| 162 | LI:1086181.1:2000SEP08 | Liver - 100% |
| 163 | LI:1164493.1:2000SEP08 | Urinary Tract - 52%, Female Genitalia - 20%, Digestive System - 12%, Male |
| | | Genitalia - 12% |
| 164 | LI:1175097.1:2000SEP08 | Unclassified/Mixed - 57%, Digestive System - 21%, Nervous System - 21% |
| 165 | LI:1092948.1:2000SEP08 | Embryonic Structures - 26%, Connective Tissue - 19%, Musculoskeletal |
| | | System - 17% |
| 166 | LI:380378.2:2000SEP08 | Nervous System - 100% |
| 167 | LI:1029674.1:2000SEP08 | Digestive System - 42%, Nervous System - 36%, Unclassified/Mixed - 22% |
| 169 | LI:1186208.1:2000SEP08 | Stomatognathic System - 51%, Sense Organs - 38% |
| 170 | LI:1170753.1:2000SEP08 | Endocrine System - 95% |
| 171 | LI:1180908.1:2000SEP08 | Skin - 21%, Embryonic Structures - 14%, Male Genitalia - 13% |
| 172 | LI:1182900.2:2000SEP08 | Digestive System - 47%, Pancreas - 34%, Respiratory System - 19% |
| 173 | LI:1169548.2:2000SEP08 | Nervous System - 100% |
| 174 | LI:1039974.1:2000SEP08 | Female Genitalia - 31%, Urinary Tract - 29%, Nervous System - 16% |
| 175 | LI:1175765.2:2000SEP08 | Nervous System - 100% |
| 176 | LI:313948.1:2000SEP08 | Unclassified/Mixed - 31%, Endocrine System - 23%, Cardiovascular |
| | | System - 19% |
| 177 | LI:335923.2:2000SEP08 | Germ Cells - 74%, Male Genitalia - 26% |
| 178 | LI:345884.1:2000SEP08 | Respiratory System - 75%, Hemic and Immune System - 25% |
| 179 | LI:417127.1:2000SEP08 | Connective Tissue - 100% |
| 180 | LI:451710.1:2000SEP08 | Connective Tissue - 90%, Nervous System - 10% |
| 181 | LI:406882.2:2000SEP08 | Sense Organs - 34%, Unclassified/Mixed - 15%, Nervous System - 15% |
| 182 | LI:728223.1:2000SEP08 | Nervous System - 100% |
| 183 | LI:289783.19:2000SEP08 | Digestive System - 38%, Unclassified/Mixed - 15%, Respiratory System - |
| | | 12% |
| 184 | LI:235255.8:2000SEP08 | Exocrine Glands - 23%, Connective Tissue - 12%, Female Genitalia - 11% |
| 185 | LI:237693.5:2000SEP08 | Urinary Tract - 32%, Hemic and Immune System - 25%, Endocrine System - |
| | | 21% |
| 186 | LI:433670.3:2000SEP08 | Nervous System - 50%, Male Genitalia - 25%, Digestive System - 25% |
| 187 | LI:202943.4:2000SEP08 | Embryonic Structures - 42%, Liver - 19%, Unclassified/Mixed - 16% |
| 188 | LI:068682.1:2000SEP08 | Unclassified/Mixed - 47%, Digestive System - 21%, Male Genitalia - 19% |
| 189 | LI:203301.3:2000SEP08 | Germ Cells - 19%, Male Genitalia - 14%, Respiratory System - 12% |
| 190 | LI:020726.3:2000SEP08 | Sense Organs - 79% |
| 191 | LI:027209.1:2000SEP08 | Musculoskeletal System - 53%, Female Genitalia - 27%, Exocrine Glands - |
| | | 17% |
| 192 | LI:108819.1:2000SEP08 | Urinary Tract - 45%, Digestive System - 28%, Skin - 15% |
| 193 | LI:021759.1:2000SEP08 | Hemic and Immune System - 40%, Nervous System - 22%, Liver - 11% |
| 194 | LI:1165967.1:2000SEP08 | Liver - 50%, Stomatognathic System - 50% |
| 195 | LI:1166315.1:2000SEP08 | Cardiovascular System - 33%, Urinary Tract - 27%, Female Genitalia - |
| | | 20%, Hemic and Immune System - 20% |
| 196 | LI:204626.1:2000SEP08 | Digestive System - 58%, Nervous System - 15%, Exocrine Glands - 13% |
| 197 | LI:801140.1:2000SEP08 | Embryonic Structures - 50%, Cardiovascular System - 38% |
| 198 | LI:286639.1:2000SEP08 | Germ Cells - 64% |
| 199 | LI:288905.4:2000SEP08 | Unclassified/Mixed - 76%, Nervous System - 24% |
| 200 | LI:332161.1:2000SEP08 | Cardiovascular System - 47%, Nervous System - 14%, Connective Tissue - |
| | | 10% |
| 201 | LI:184867.1:2000SEP08 | Unclassified/Mixed - 33%, Embryonic Structures - 24%, Male Genitalia - |
| | | 16% |
| 202 | LI:229932.4:2000SEP08 | Musculoskeletal System - 40%, Cardiovascular System - 21% |
| 203 | LI:1189932.1:2000SEP08 | Embryonic Structures - 37%, Germ Cells - 18%, Sense Organs - 13% |
| 204 | LI:1076689.1:2000SEP08 | Liver - 99% |
| 205 | LI:415181.2:2000SEP08 | Nervous System - 100% |
| 206 | LI:296358.1:2000SEP08 | Hemic and Immune System - 83% |
| 207 | LI:205186.3:2000SEP08 | Cardiovascular System - 59%, Male Genitalia - 20%, Unclassified/Mixed - |
| | | 16% |
| 208 | LI:220537.2:2000SEP08 | Stomatognathic System - 52%, Male Genitalia - 32% |
| 209 | LI:248364.2:2000SEP08 | Cardiovascular System - 30%, Female Genitalia - 23%, Sense Organs - |
| | | 22% |
| 210 | LI:2048338.1:2000SEP08 | Connective Tissue - 90%, Nervous System - 10% |
| 211 | LI:1185203.8:2000SEP08 | Nervous System - 75%, Female Genitalia - 25% |
| 212 | LI:021770.3:2000SEP08 | Pancreas - 77% |
| 213 | LI:1185841.1:2000SEP08 | Hemic and Immune System - 30%, Respiratory System - 26% |
| 214 | LI:1181710.1:2000SEP08 | Male Genitalia - 38%, Nervous System - 38%, Hemic and Immune System - |
| | | 25% |
| 215 | LI:2048959.1:2000SEP08 | Musculoskeletal System - 73%, Endocrine System - 27% |
| 216 | LI:798494.1:2000SEP08 | Germ Cells - 88% |
| 217 | LI:2049223.1:2000SEP08 | Male Genitalia - 60%, Hemic and Immune System - 40% |
| 218 | LI:1177833.1:2000SEP08 | Liver - 55%, Embryonic Structures - 10% |
| 219 | LI:2049267.1:2000SEP08 | Cardiovascular System - 36%, Urinary Tract - 29%, Digestive System - 21% |
| 220 | LI:1165939.1:2000SEP08 | Sense Organs - 62%, Endocrine System - 20% |
| 221 | LI:1170958.1:2000SEP08 | Exocrine Glands - 53%, Cardiovascular System - 26%, Male Genitalia - |
| | | 16% |
| 222 | LI:1089827.1:2000SEP08 | Endocrine System - 16%, Male Genitalia - 13%, Musculoskeletal System - |
| | | 12% |
| 223 | LI:792112.1:2000SEP08 | Liver - 83% |
| 224 | LI:282219.2:2000SEP08 | Embryonic Structures - 55%, Exocrine Glands - 23%, Male Genitalia - 14% |
| 225 | LI:1088010.2:2000SEP08 | Exocrine Glands - 31%, Musculoskeletal System - 23%, Respiratory System - |
| | | 13% |
| 226 | LI:1165276.1:2000SEP08 | Nervous System - 18%, Male Genitalia - 16%, Pancreas - 16% |
| 227 | LI:1169524.2:2000SEP08 | Endocrine System - 41%, Urinary Tract - 16%, Musculoskeletal System - |
| | | 14%, Male Genitalia - 14% |
| 228 | LI:1180255.1:2000SEP08 | Female Genitalia - 66%, Liver - 11%, Embryonic Structures - 11% |
| 229 | LI:1091903.1:2000SEP08 | Germ Cells - 93% |
| 230 | LI:1169219.1:2000SEP08 | Sense Organs - 34%, Skin - 22% |
| 231 | LI:2050313,1:2000SEP08 | Skin - 15%, Respiratory System - 14%, Male Genitalia - 14% |
| 232 | LI:209351.3:2000SEP08 | Cardiovascular System - 41% |
| 233 | LI:119900.1:2000SEP08 | Stomatognathic System - 77% |
| 234 | LI:2052274.1:2000SEP08 | Urinary Tract - 31%, Female Genitalia - 19%, Liver - 12% |
| 235 | LI:1075502.1:2000SEP08 | Liver - 100% |
| 236 | LI:813697.1:2000SEP08 | Endocrine System - 36%, Unclassified/Mixed - 25%, Exocrine Glands - 15% |
| 237 | LI:814261.1:2000SEP08 | Exocrine Glands - 50%, Respiratory System - 20%, Urinary Tract - 13% |
| 238 | LI:775334.1:2000SEP08 | Cardiovascular System - 26%, Embryonic Structures - 23%, |
| | | Unclassified/Mixed - 15% |
| 239 | LI:1180325.1:2000SEP08 | Skin - 38%, Pancreas - 23%, Female Genitalia - 23% |
| 240 | LI:1183147.3:2000SEP08 | Female Genitalia - 42%, Respiratory System - 17%, Nervous System - 13% |
| 241 | LI:1175373.3:2000SEP08 | Nervous System - 50%, Male Genitalia - 25%, Digestive System - 25% |
| 242 | LI:813757.1:2000SEP08 | Female Genitalia - 50%, Nervous System - 50% |
| 243 | LI:1182979.2:2000SEP08 | Respiratory System - 67%, Pancreas - 19% |
| 244 | LI:1177823.2:2000SEP08 | Embryonic Structures - 48%, Cardiovascular System - 20%, Exocrine |
| | | Glands - 20% |
| 245 | LI:1174279.1:2000SEP08 | Endocrine System - 36%, Nervous System - 19%, Liver - 18% |
| 246 | LI:1178411.1:2000SEP08 | Nervous System - 21%, Liver- 18%, Exocrine Glands - 15% |
| 247 | LI:1182739.1:2000SEP08 | Unclassified/Mixed - 34%, Skin - 18%, Nervous System - 12% |
| 248 | LI:234937.4:2000SEP08 | Endocrine System - 33%, Nervous System - 24%, Digestive System - 21% |
| 249 | LI:1170660.1:2000SEP08 | Musculoskeletal System - 21%, Nervous System - 18%, Cardiovascular |
| | | System - 13%, Male Genitalia - 13% |
| 250 | LI:1144409.1:2000SEP08 | Sense Organs - 53% |
| 251 | LI:246290.10:2000SEP08 | Cardiovascular System - 29%, Female Genitalia - 29% |
| 252 | LI:280034.1:2000SEP08 | Female Genitalia - 100% |

| SEQ ID NO: | Frame | Length | Start | Stop | GI Number | Probability | Score Annotation |
|---|---|---|---|---|---|---|---|
| 253 | 2 | 114 | 2 | 343 | g11643582 | 2.00E−68 | PR-domain containing protein 14 |
| 253 | 2 | 114 | 2 | 343 | g10434076 | 2.00E−68 | unnamed protein product |
| 253 | 2 | 114 | 2 | 343 | g7020503 | 4.00E−27 | unnamed protein product |
| 255 | 2 | 211 | 2 | 634 | g7243243 | 2.00E−43 | KIAA1431 protein |
| 255 | 2 | 211 | 2 | 634 | g4567178 | 2.00E−41 | R31665_2 (AA 1-673) |
| 255 | 2 | 211 | 2 | 634 | g3445181 | 2.00E−41 | R31665_2 |
| 256 | 3 | 151 | 54 | 506 | g10433955 | 5.00E−40 | unnamed protein product |
| 256 | 3 | 151 | 54 | 506 | g7295442 | 4.00E−14 | CG17334 gene product |
| 256 | 3 | 151 | 54 | 506 | g2073111 | 3.00E−11 | Y box protein 2 |
| 257 | 2 | 138 | 17 | 430 | g12407395 | 4.00E−54 | tripartite motif protein TRIM7 |
| 257 | 2 | 138 | 17 | 430 | g12407397 | 2.00E−42 | tripartite motif protein TRIM7 |
| 257 | 2 | 138 | 17 | 430 | g12407379 | 8.00E−16 | tripartite motif protein TRIM4 isoform beta |
| 258 | 1 | 317 | 1 | 951 | g9246977 | 1.00E−137 | RNA-binding protein BRUNOL4 |
| 258 | 1 | 317 | 1 | 951 | g12746394 | 1.00E−136 | CUG-BP and ETR-3 like factor 4 |
| 258 | 1 | 317 | 1 | 951 | g13278792 | 1.00E−106 | Bruno (Drosophila)-like 4, RNA binding protein |
| 260 | 2 | 114 | 14 | 355 | g4589588 | 2.00E−34 | KIAA0972 protein |
| 260 | 2 | 114 | 14 | 355 | g7576272 | 2.00E−30 | bA393J16.1 (zinc finger protein 33a (KOX 31)) |
| 260 | 2 | 114 | 14 | 355 | g498152 | 2.00E−30 | ha0946 protein is Kruppel-related. |
| 261 | 2 | 127 | 140 | 520 | g10434195 | 2.00E−64 | unnamed protein product |
| 261 | 2 | 127 | 140 | 520 | g13529188 | 4.00E−42 | Unknown (protein for MGC: 12466) |
| 261 | 2 | 127 | 140 | 520 | g6467206 | 3.00E−36 | gonadotropin inducible transcription repressor-4 |
| 262 | 1 | 151 | 1 | 453 | g14042293 | 4.00E−47 | unnamed protein product |
| 262 | 1 | 151 | 1 | 453 | g12052983 | 1.00E−46 | hypothetical protein |
| 262 | 1 | 151 | 1 | 453 | g487785 | 7.00E−46 | zinc finger protein ZNF136 |
| 263 | 1 | 79 | 16 | 252 | g349075 | 4.00E−16 | calmodulin-binding protein |
| 263 | 1 | 79 | 16 | 252 | g13543326 | 4.00E−16 | hypothetical protein MGC8407 |
| 263 | 1 | 79 | 16 | 252 | g12804937 | 4.00E−16 | Unknown (protein for MGC: 3732) |
| 264 | 1 | 183 | 112 | 660 | g13325337 | 3.00E−83 | Unknown (protein for MGC: 10520) |
| 264 | 1 | 183 | 112 | 660 | g8439407 | 3.00E−22 | zinc finger protein |
| 264 | 1 | 183 | 112 | 660 | g7020166 | 3.00E−22 | unnamed protein product |
| 266 | 3 | 260 | 3 | 782 | g12698001 | 1.00E−147 | KIAA1728 protein |
| 266 | 3 | 260 | 3 | 782 | g8052233 | 1.00E−94 | putative ankyrin-repeat containing protein |
| 266 | 3 | 260 | 3 | 782 | g7294632 | 2.00E−61 | CG5679 gene product |
| 267 | 2 | 196 | 2 | 589 | g6249687 | 1.00E−108 | P31155_1 |
| 267 | 2 | 196 | 2 | 589 | g10436360 | 1.00E−53 | unnamed protein product |
| 267 | 2 | 196 | 2 | 589 | g14042803 | 6.00E−50 | unnamed protein product |
| 268 | 1 | 113 | 130 | 468 | g14596085 | 7.00E−55 | (AY042830) Putative 40S ribosomal protein S15A |
| 268 | 1 | 113 | 130 | 468 | g9757906 | 7.00E−55 | 40S ribosomal protein S15A |
| 268 | 1 | 113 | 130 | 468 | g8439890 | 7.00E−55 | Strong similarity to 40S ribosomal protein S15A |
| | | | | | | | from Arabidopsis thallana gb\|L27461. EST |
| | | | | | | | gb\|R30315 comes from this gene. |
| 269 | 3 | 165 | 3 | 497 | g12052983 | 2.00E−73 | hypothetical protein |
| 269 | 3 | 165 | 3 | 497 | g5262560 | 1.00E−47 | hypothetical protein |
| 269 | 3 | 165 | 3 | 497 | g10434856 | 1.00E−47 | unnamed protein product |
| 270 | 3 | 168 | 165 | 668 | g12052983 | 4.00E−70 | hypothetical protein |
| 270 | 3 | 168 | 165 | 668 | g5262560 | 4.00E−35 | hypothetical protein |
| 270 | 3 | 168 | 165 | 668 | g10434856 | 5.00E−34 | unnamed protein product |
| 271 | 2 | 122 | 2 | 367 | g12044553 | 8.00E−48 | bA261P9.2 (putative novel protein similar to fly |
| | | | | | | | CG7340 and human putative aminopeptidase |
| | | | | | | | ZK353.6 in chromosome 3 (EC 3.4.11.-)) |
| 271 | 2 | 122 | 2 | 367 | g10432867 | 5.00E−47 | unnamed protein product |
| 271 | 2 | 122 | 2 | 367 | g7299691 | 5.00E−42 | BcDNA: LD41548 gene product (alt 1) |
| 272 | 2 | 91 | 212 | 484 | g872315 | 3.00E−35 | 40S ribosomal protein S12 |
| 272 | 2 | 91 | 212 | 484 | g12842004 | 3.00E−35 | putative |
| 272 | 2 | 91 | 212 | 484 | g12833134 | 3.00E−35 | putative |
| 273 | 3 | 225 | 3 | 677 | g510552 | 1.00E−100 | ribosomal protein L13 |
| 273 | 3 | 225 | 3 | 677 | g12833027 | 1.00E−99 | putative |
| 273 | 3 | 225 | 3 | 677 | g3869148 | 4.00E−98 | robosomal protein L13 |
| 274 | 3 | 153 | 3 | 461 | g825539 | 2.00E−84 | MLC2 |
| 274 | 3 | 153 | 3 | 461 | g1675396 | 2.00E−84 | myosin light chain 2 |
| 274 | 3 | 153 | 3 | 461 | g1637 | 2.00E−84 | myosin light chain 2 type 2 |
| 275 | 2 | 296 | 176 | 1063 | g12407387 | 1.00E−147 | tripartite motif protein TRIM5 isoform delta |
| 275 | 2 | 296 | 176 | 1063 | g12407385 | 1.00E−47 | tripartite motif protein TRIM5 isoform gamma |
| 275 | 2 | 296 | 176 | 1063 | g12407383 | 1.00E−147 | tripartite motif protein TRIM5 isoform beta |
| 276 | 1 | 175 | 22 | 546 | g13752754 | 1.00E−26 | zinc finger 1111 |
| 276 | 1 | 175 | 22 | 546 | g14348588 | 5.00E−25 | KRAB zinc finger protein |
| 276 | 1 | 175 | 22 | 546 | g12654015 | 4.00E−24 | Similar to hypothetical protein FLJ10891 |
| 277 | 3 | 76 | 141 | 368 | g9714522 | 5.00E−13 | bA162G10.3 (zinc finger protein) |
| 277 | 3 | 76 | 141 | 368 | g5730194 | 5.00E−13 | KRAB protein domain |
| 277 | 3 | 76 | 141 | 368 | g4589588 | 8.00E−13 | KIAA0972 protein |
| 278 | 3 | 146 | 624 | 1061 | g12697318 | 5.00E−14 | PBX4 protein |
| 278 | 3 | 146 | 624 | 1061 | g7160798 | 9.00E−13 | pbxy homeodomain protein |
| 278 | 3 | 146 | 624 | 1061 | g634053 | 9.00E−13 | PBX2 |
| 279 | 3 | 391 | 3 | 1175 | g10439850 | 1.00E−116 | unnamed protein product |
| 279 | 3 | 391 | 3 | 1175 | g9968290 | 1.00E−112 | zinc finger protein 304 |
| 279 | 3 | 391 | 3 | 1175 | g14249844 | 1.00E−111 | Similar to hypothetical protein FLJ23233 |
| 280 | 3 | 80 | 156 | 395 | g12052983 | 7.00E−15 | hypothetical protein |
| 280 | 3 | 80 | 156 | 395 | g13277768 | 1.00E−11 | zinc finger protein 93 |
| 280 | 3 | 80 | 156 | 395 | g1184371 | 1.00E−11 | zinc finger protein; Method: conceptual |
| | | | | | | | translation supplied by author |
| 281 | 2 | 162 | 74 | 559 | g6467206 | 8.00E−37 | gonadotropin inducible transcription repressor-4 |
| 281 | 2 | 162 | 74 | 559 | g9187356 | 2.00E−36 | hypothetical protein, similar to (AB021644) |
| | | | | | | | GONADOTROPIN INDUCIBLE |
| | | | | | | | TRANSCRIPTION REPRESSOR-4 |
| 281 | 2 | 162 | 74 | 559 | g220637 | 2.00E−36 | zinc finger protein |
| 282 | 2 | 164 | 50 | 541 | g13752754 | 1.00E−28 | zinc finger 1111 |
| 282 | 2 | 164 | 50 | 541 | g14348588 | 5.00E−28 | KRAB zinc finger protein |
| 282 | 2 | 164 | 50 | 541 | g12654015 | 3.00E−27 | Similar to hypothetical protein FLJ10891 |
| 283 | 3 | 105 | 75 | 389 | g12052732 | 2.00E−38 | hypothetical protein |
| 283 | 3 | 105 | 75 | 389 | g3329372 | 9.00E−37 | DNA-binding protein |
| 283 | 3 | 105 | 75 | 389 | g7959207 | 3.00E−34 | KIAA1473 protein |
| 284 | 1 | 123 | 289 | 657 | g14042035 | 9.00E−64 | unnamed protein product |
| 284 | 1 | 123 | 289 | 657 | g6224922 | 1.00E−41 | BTB/POZ domain zinc finger factor HOF-S |
| 284 | 1 | 123 | 289 | 657 | g6063139 | 1.00E−41 | BTB/POZ domain zinc finger factor HOF-L |
| 285 | 1 | 174 | 199 | 720 | g7022603 | 2.00E−20 | unnamed protein product |
| 285 | 1 | 174 | 199 | 720 | g7022652 | 3.00E−10 | unnamed protein product |
| 285 | 1 | 174 | 199 | 720 | g3283350 | 3.00E−08 | calmodulin-binding protein SHA1 |
| 286 | 2 | 181 | 71 | 613 | g13591714 | 4.00E−97 | (AF343664) immunoglobulin superfamily |
| | | | | | | | receptor translocation associated |
| | | | | | | | protein 2c |
| 286 | 2 | 181 | 71 | 613 | g13591712 | 4.00E−97 | (AF343663) immunoglobulin superfamily receptor |
| | | | | | | | translocation |
| | | | | | | | associated protein 2b |
| 286 | 2 | 181 | 71 | 613 | g13591710 | 4.00E−97 | (AF343662) immunoglobulin superfamily receptor |
| | | | | | | translocation | |
| | | | | | | | associated protein 2a |
| 287 | 1 | 91 | 64 | 336 | g10047183 | 8.00E−48 | KIAA1559 protein |
| 287 | 1 | 91 | 64 | 336 | g5080758 | 8.00E−25 | BC331191_1 |
| 287 | 1 | 91 | 64 | 336 | g456269 | 1.00E−21 | zinc finger protein 30 |
| 288 | 1 | 188 | 226 | 789 | g11999277 | 4.00E−45 | solute carrier |
| 288 | 1 | 188 | 226 | 789 | g12845461 | 8.00E−34 | putative |
| 288 | 1 | 188 | 226 | 789 | g10434874 | 2.00E−33 | unnamed protein product |
| 289 | 2 | 290 | 2 | 871 | g14017771 | 1.00E−157 | fibrillin3 |
| 289 | 2 | 290 | 2 | 871 | g762831 | 1.00E−113 | fibrillin 2 |
| 289 | 2 | 290 | 2 | 871 | g4959652 | 1.00E−113 | flbrillin-2 |
| 290 | 3 | 199 | 3 | 599 | g3757719 | 1.00E−83 | dJ422F24.1 (PUTATIVE novel protein |
| | | | | | | | similar to C. elegans C0202.5) |
| 290 | 3 | 199 | 3 | 599 | g12832288 | 5.00E−83 | putative |
| 290 | 3 | 199 | 3 | 599 | g7294769 | 2.00E−38 | CG6279 gene product |
| 291 | 3 | 148 | 3 | 446 | g13489168 | 5.00E−77 | 60S ribosomal protein L17 |
| 291 | 3 | 148 | 3 | 446 | g13430182 | 3.00E−76 | ribosomal protein L17 |
| 291 | 3 | 148 | 3 | 446 | g14596111 | 2.00E−75 | (AY042843) 60S ribosomal protein L17 |
| 292 | 1 | 92 | 37 | 312 | g6002102 | 5.00E−39 | Acyl-CoA binding protein (ACBP) |
| 292 | 1 | 92 | 37 | 312 | g1938236 | 3.00E−38 | acyl-CoA-binding protein |
| 292 | 1 | 92 | 37 | 312 | g6002104 | 2.00E−37 | Acyl-CoA binding protein (ACBP) |
| 293 | 3 | 256 | 3 | 770 | g5091520 | 1.00E−127 | ESTs AU058081(E30812), AU058365(E50679), |
| | | | | | | | AU030138(E50679) correspond |
| | | | | | | | to a region of the predicted gene.; |
| | | | | | | | Similar to Spinacia oleracea mRNA for |
| | | | | | | | proteasome 37 kD subunit, (X96974) |
| 293 | 3 | 256 | 3 | 770 | g8671496 | 1.00E−127 | alpha 3 subunit of 20S proteasome |
| 293 | 3 | 256 | 3 | 770 | g8096329 | 1.00E−127 | ESTs AU058081(E3082), AU075427 |
| | | | | | | | (E30384) correspond to a region of the |
| | | | | | | | predicted gene, ˜Similar to Spinacia |
| | | | | | | | oleracea proteasome 27 kD subunit (P52427) |
| 295 | 3 | 318 | 132 | 1085 | g11602755 | 1.00E−37 | zinc finger protein |
| 295 | 3 | 318 | 132 | 1085 | g12843135 | 6.00E−33 | putative |
| 295 | 3 | 318 | 132 | 1085 | g13094151 | 3.00E−25 | zinc finger protein hRit1 alpha |
| 296 | 1 | 194 | 304 | 885 | g12856559 | 1.00E−85 | putative |
| 296 | 1 | 194 | 304 | 885 | g12856631 | 3.00E−85 | putative |
| 296 | 1 | 194 | 304 | 885 | g12849896 | 3.00E−85 | putative |
| 297 | 1 | 469 | 1 | 1407 | g9927838 | 0 | unnamed protein product |
| 297 | 1 | 469 | 1 | 1407 | g10045028 | 0 | unnamed protein product |
| 297 | 1 | 469 | 1 | 1407 | g10047295 | 1.00E−169 | KIAA1610 protein |
| 298 | 3 | 81 | 3 | 245 | g13249093 | 5.00E−37 | carbonic anhydrase XIII |
| 298 | 3 | 81 | 3 | 245 | g12845416 | 5.00E−37 | putative |
| 298 | 3 | 81 | 3 | 245 | g65332 | 4.00E−22 | carbonic anhydrase |
| 299 | 1 | 452 | 1 | 1356 | g11177164 | 0 | polydom protein |
| 299 | 1 | 452 | 1 | 1356 | g12060830 | 1.00E−142 | serologically defined breast cancer antigen |
| | | | | | | | NY-BR-38 |
| 299 | 1 | 452 | 1 | 1356 | g7292728 | 1.00E−52 | fw gene product |
| 300 | 1 | 362 | 97 | 1182 | g14594722 | 0 | (AY037298) elongation of very long chain fatty |
| | | | | | | | acids protein |
| 300 | 1 | 362 | 97 | 1182 | g12044051 | 0 | ELOVL4 |
| 300 | 1 | 362 | 97 | 1182 | g12044043 | 0 | ELOVL4 |
| 301 | 2 | 354 | 2 | 1063 | g14272764 | 1.00E−146 | unnamed protein product |
| 301 | 2 | 354 | 2 | 1063 | g561853 | 5.00E−28 | megalin |
| 301 | 2 | 354 | 2 | 1063 | g1809240 | 7.00E−27 | gp330 precursor |
| 302 | 1 | 305 | 220 | 1134 | g12483902 | 1.00E−83 | zinc finger protein HIT-10 |
| 302 | 1 | 305 | 220 | 1134 | g6002480 | 4.00E−49 | BWSCR2 associated zinc-finger protein BAZ2 |
| 302 | 1 | 305 | 220 | 1134 | g9963806 | 4.00E−47 | zinc finger protein ZNF287 |
| 303 | 2 | 659 | 164 | 2140 | g14587851 | 0 | (AB050785) Graf2 |
| 303 | 2 | 659 | 164 | 2140 | g13310137 | 0 | PSGAP-m |
| 303 | 2 | 659 | 164 | 2140 | g13310135 | 0 | PSGAP-s |
| 304 | 3 | 390 | 102 | 1271 | g13879442 | 1.00E−170 | Similar to RIKEN cDNA 2310035M22 gene |
| 304 | 3 | 390 | 102 | 1271 | g12855490 | 1.00E−165 | putative |
| 304 | 3 | 390 | 102 | 1271 | g12848905 | 1.00E−102 | putative |
| 305 | 2 | 294 | 2 | 883 | g13898617 | 1.00E−142 | serine/threonine protein kinase SSTK |
| 305 | 2 | 294 | 2 | 883 | g13540326 | 1.00E−142 | serine/threonine kinase FKSG82 |
| 305 | 2 | 294 | 2 | 883 | g13898619 | 1.00E−140 | serine/threonine protein kinase SSTK |
| 306 | 1 | 269 | 1 | 807 | g14250138 | 1.00E−139 | Similar to RIKEN cDNA 5730421E18 gene |
| 306 | 1 | 269 | 1 | 807 | g12856916 | 1.00E−120 | putative |
| 306 | 1 | 269 | 1 | 807 | g12840367 | 1.00E−120 | putative |
| 307 | 3 | 270 | 393 | 1202 | g10434082 | 1.00E−103 | unnamed protein product |
| 307 | 3 | 270 | 393 | 1202 | g12052816 | 1.00E−102 | hypothetical protein |
| 307 | 3 | 270 | 393 | 1202 | g297157 | 1.00E−74 | rab17 |
| 308 | 3 | 358 | 150 | 1223 | g12861800 | 1.00E−170 | putative |
| 308 | 3 | 358 | 150 | 1223 | g3878713 | 5.00E−73 | (Z46935) weak similarity with quinone |
| | | | | | | | oxidoreductase, contains similarity to |
| | | | | | | | Pfam domain: PF00107 (Zinc-binding |
| | | | | | | | dehydrogenases), Score = −80.6, E- |
| | | | | | | | value = 6.2e−06, N = 1˜cDNA |
| | | | | | | | EST yk164b4.5 comes from this gene˜ |
| | | | | | | | cDNA EST yk164b4.3 comes from this |
| | | | | | | | gene˜cDNA EST yk264f3.5 comes from |
| | | | | | | | this |
| 308 | 3 | 358 | 150 | 1223 | g2633069 | 2.00E−52 | similar to quinone oxidoreductase |
| 309 | 2 | 232 | 2 | 697 | g12834244 | 1.00E−71 | putative |
| 309 | 2 | 232 | 2 | 697 | g12833174 | 1.00E−71 | putative |
| 309 | 2 | 232 | 2 | 697 | g13905260 | 1.00E−36 | RIKEN cDNA 1300006C06 gene |
| 310 | 3 | 184 | 3 | 554 | g2335037 | 9.00E−67 | Tim17 |
| 310 | 3 | 184 | 3 | 554 | g4378524 | 2.00E−65 | mitochondrial inner membrane translocase |
| | | | | | | | component Tlm17a |
| 310 | 3 | 184 | 3 | 554 | g12833600 | 2.00E−65 | putative |
| 311 | 1 | 206 | 46 | 663 | g12314268 | 4.00E−82 | dJ14N1.2 (novel S-100/ICaBP type calcium |
| | | | | | | | binding domain protein, similar |
| | | | | | | | to trichohyalin) |
| 311 | 1 | 206 | 46 | 663 | g553621 | 3.00E−14 | profilaggrin |
| 311 | 1 | 206 | 46 | 663 | g12314267 | 3.00E−14 | dJ14N1.1.1 (profilaggrin 5′ end) |
| 312 | 2 | 301 | 59 | 961 | g12314195 | 1.00E−171 | bA255A11.3 (novel protein similar to KIAA1074) |
| 312 | 2 | 301 | 59 | 961 | g12314164 | 1.00E−134 | bA526D8.2 (novel protein similar to KIAA1074) |
| 312 | 2 | 301 | 59 | 961 | g12053099 | 4.00E−95 | hypothetical protein |
| 313 | 1 | 156 | 70 | 537 | g6692607 | 2.00E−65 | MGA protein |
| 313 | 1 | 156 | 70 | 537 | g5931585 | 1.00E−44 | T-box family member; T-box domain |
| 313 | 1 | 156 | 70 | 537 | g4049463 | 4.00E−16 | transcription factor TBX6 |
| 314 | 1 | 279 | 340 | 1176 | g12854977 | 1.00E−123 | putative |
| 314 | 1 | 279 | 340 | 1176 | g7267246 | 1.00E−41 | putative adenosine deaminase |
| 314 | 1 | 279 | 340 | 1176 | g7299138 | 2.00E−39 | CG11994 gene product |
| 315 | 1 | 329 | 181 | 1167 | g4582324 | 0 | dJ708F5.1 (PUTATIVE novel Collagen alpha 1 |
| | | | | | | | LIKE protein) |
| 315 | 1 | 329 | 181 | 1167 | g12052774 | 0 | hypothetical protein |
| 315 | 1 | 329 | 181 | 1167 | g2326442 | 1.00E−38 | collagen type XII alpha 1 chain |
| 316 | 3 | 757 | 3 | 2273 | g8896164 | 0 | kinesin-like protein GAKIN |
| 316 | 3 | 757 | 3 | 2273 | g10697238 | 0 | KIF13A |
| 316 | 3 | 757 | 3 | 2273 | g12054032 | 0 | KINESIN-13A2 |
| 317 | 1 | 329 | 1 | 987 | g14595658 | 8.00E−97 | (AF387815) LIM protein prickle |
| 317 | 1 | 329 | 1 | 987 | g9229890 | 3.00E−84 | prickle 2 |
| 317 | 1 | 329 | 1 | 987 | g9229888 | 3.00E−83 | prickle 1 |
| 318 | 1 | 494 | 1 | 1482 | g1763096 | 0 | glutamate pyruvate transaminase |
| 318 | 1 | 494 | 1 | 1482 | g467528 | 0 | alanine aminotransferase |
| 318 | 1 | 494 | 1 | 1482 | g1507680 | 0 | alanine aminotransferase |
| 319 | 1 | 156 | 400 | 867 | g5912051 | 2.00E−30 | hypothetical protein |
| 319 | 1 | 156 | 400 | 867 | g14278937 | 2.00E−30 | calmin |
| 319 | 1 | 156 | 400 | 867 | g10437695 | 2.00E−30 | unnamed protein product |
| 320 | 1 | 128 | 118 | 501 | g7294107 | 4.00E−44 | CG4638 gene product |
| 320 | 1 | 128 | 118 | 501 | g3169065 | 3.00E−40 | elongation factor 2-like protein |
| 320 | 1 | 128 | 118 | 501 | g1302132 | 4.00E−38 | ORF YNL163c |
| 321 | 1 | 197 | 151 | 741 | g13185203 | 2.00E−21 | unnamed protein product |
| 321 | 1 | 197 | 151 | 741 | g2463632 | 3.00E−14 | monocarboxylate transporter homologue MCT6 |
| 321 | 1 | 197 | 151 | 741 | g6103363 | 3.00E−13 | monocarboxylate transporter MCT3 |
| 322 | 1 | 560 | 1 | 1680 | g4240293 | 1.00E−141 | KIAA0902 protein |
| 322 | 1 | 560 | 1 | 1680 | g4151807 | 1.00E−141 | membrane-associated guanylate kinase- |
| | | | | | | | interacting protein 2 Maguin-2 |
| 322 | 1 | 560 | 1 | 1680 | g4151805 | 1.00E−141 | membrane-associated guanylate kinase- |
| | | | | | | | interacting protein 1 Maguin-1 |
| 323 | 2 | 163 | 2 | 490 | g10172680 | 3.00E−06 | stage V sporulation protein C |
| | | | | | | | (peptidyl-tRNA hydrolase) |
| 323 | 2 | 163 | 2 | 490 | g1001232 | 4.00E−06 | (D64003) peptidyl-tRNA hydrolase |
| 323 | 2 | 163 | 2 | 490 | g2983032 | 1.00E−05 | peptidyl-tRNA hydrolase |
| 324 | 2 | 197 | 2 | 592 | g9295345 | 2.00E−67 | HSKM-B |
| 324 | 2 | 197 | 2 | 592 | g12834773 | 5.00E−19 | putative |
| 324 | 2 | 197 | 2 | 592 | g5870834 | 3.00E−17 | skm-BOP2 |
| 325 | 3 | 520 | 3 | 1562 | g8655678 | 1.00E−139 | hypothetical protein |
| 325 | 3 | 520 | 3 | 1562 | g12382779 | 1.00E−59 | zinc transporter 1 |
| 325 | 3 | 520 | 3 | 1562 | g577843 | 2.00E−59 | ZnT-1 |
| 326 | 1 | 772 | 640 | 2955 | g13358642 | 0 | hypothetical protein |
| 326 | 1 | 772 | 640 | 2955 | g10435064 | 0 | unnamed protein product |
| 326 | 1 | 772 | 640 | 2955 | g10434826 | 0 | unnamed protein product |
| 327 | 3 | 702 | 153 | 2258 | g7264026 | 0 | dJ876B10.2 (novel protein (ortholog of rat |
| | | | | | | | EXO84)) |
| 327 | 3 | 702 | 153 | 2258 | g2827164 | 0 | exo84 |
| 327 | 3 | 702 | 153 | 2258 | g7301432 | 2.00E−38 | CG6095 gene product |
| 328 | 3 | 255 | 609 | 1373 | g13383265 | 1.00E−106 | actin related protein |
| 328 | 3 | 255 | 609 | 1373 | g13938319 | 1.00E−105 | Unknown (protein for MGC: 15664) |
| 328 | 3 | 255 | 609 | 1373 | g12840619 | 9.00E−72 | putative |
| 330 | 2 | 178 | 203 | 736 | g10435210 | 2.00E−76 | unnamed protein product |
| 330 | 2 | 178 | 203 | 736 | g14598968 | 3.00E−58 | (AX179297) 21615 ADH |
| 330 | 2 | 178 | 203 | 736 | g8895083 | 3.00E−58 | oxidoreductase UCPA |
| 331 | 1 | 217 | 1624 | 2274 | g8655648 | 1.00E−108 | hypothetical protein |
| 331 | 1 | 217 | 1624 | 2274 | g12803319 | 1.00E−108 | Unknown (protein for MGC: 3090) |
| 331 | 1 | 217 | 1624 | 2274 | g10047337 | 1.00E−108 | KIAA1630 protein |
| 332 | 1 | 191 | 823 | 1395 | g9651711 | 4.00E−72 | arsenite inducible RNA associated protein |
| 332 | 1 | 191 | 823 | 1395 | g12835478 | 3.00E−53 | putative |
| 332 | 1 | 191 | 823 | 1395 | g7295806 | 6.00E−45 | CG12795 gene product |
| 333 | 2 | 252 | 1223 | 1978 | g12856598 | 6.00E−93 | putative |
| 333 | 2 | 252 | 1223 | 1978 | g14042913 | 2.00E−19 | unnamed protein product |
| 333 | 2 | 252 | 1223 | 1978 | g14424618 | 9.00E−19 | hypothetical protein MGC2628 |
| 334 | 2 | 502 | 305 | 1810 | g12845866 | 1.00E−131 | putative |
| 334 | 2 | 502 | 305 | 1810 | g13477235 | 4.00E−80 | Similar to RIKEN cDNA 0610037N03 gene |
| 334 | 2 | 502 | 305 | 1810 | g12833017 | 6.00E−22 | putative |
| 335 | 2 | 369 | 2 | 1108 | g12856270 | 1.00E−165 | putative |
| 335 | 2 | 369 | 2 | 1108 | g6682873 | 1.00E−119 | reduced expression in cancer |
| 335 | 2 | 369 | 2 | 1108 | g7230612 | 1.00E−116 | small rec |
| 337 | 2 | 332 | 2 | 997 | g8886025 | 1.00E−166 | collapsin response mediator protein-5 |
| 337 | 2 | 332 | 2 | 997 | g8671168 | 1.00E−166 | hypothetical protein |
| 337 | 2 | 332 | 2 | 997 | g13259169 | 1.00E−166 | phosphoprotein ULIP6 |
| 338 | 1 | 215 | 73 | 717 | g12652727 | 1.00E−125 | Unknown (protein for IMAGE: 3352566) |
| 338 | 1 | 215 | 73 | 717 | g488555 | 2.00E−67 | zinc finger protein ZNF135 |
| 338 | 1 | 215 | 73 | 717 | g8050899 | 6.00E−65 | ZNF180 |
| 339 | 3 | 364 | 3 | 1094 | g9758769 | 1.00E−64 | 11-beta-hydroxysteroid dehydrogenase-like |
| 339 | 3 | 364 | 3 | 1094 | g8777393 | 1.00E−64 | 11-beta-hydroxysteroid dehydrogenase-like |
| 339 | 3 | 364 | 3 | 1094 | g8777400 | 1.00E−54 | contains similarity to oxidoreductase˜ |
| | | | | | | | gene_id: MFB16.17 |
| 340 | 3 | 229 | 21 | 707 | g12052959 | 1.00E−130 | hypothetical protein |
| 340 | 3 | 229 | 21 | 707 | g14336767 | 1.00E−128 | similar to |
| | | | | | | | homoprotocatechuate catabolism bifunctional |
| | | | | | | | isomerase/decarboxylase |
| 340 | 3 | 229 | 21 | 707 | g7670464 | 1.00E−115 | unnamed protein product |
| 341 | 1 | 164 | 235 | 726 | g2286123 | 8.00E−47 | testis specific DNAj-homolog |
| 341 | 1 | 164 | 235 | 726 | g12838392 | 8.00E−47 | putative |
| 341 | 1 | 164 | 235 | 726 | g12838396 | 4.00E−46 | putative |
| 342 | 1 | 131 | 199 | 591 | g12804323 | 5.00E−60 | Unknown (protein for MGC: 4054) |
| 342 | 1 | 131 | 199 | 591 | g3264773 | 5.00E−32 | zinc-finger protein-37; ZFP-37 |
| 342 | 1 | 131 | 199 | 591 | g10440398 | 5.00E−32 | FLJ00032 protein |
| 343 | 2 | 406 | 701 | 1918 | g12697482 | 1.00E−121 | dJ583P15.7.2 (novel zinc finger |
| | | | | | | | protein similar to rat RIN ZF) |
| 343 | 2 | 406 | 701 | 1918 | g12855580 | 1.00E−81 | putative |
| 343 | 2 | 406 | 701 | 1918 | g12847599 | 7.00E−78 | putative |
| 344 | 3 | 219 | 3 | 659 | g6091722 | 3.00E−51 | putative ribosomal protein L13 |
| 344 | 3 | 219 | 3 | 659 | g6650751 | 2.00E−49 | ribosomal protein I |
| 344 | 3 | 219 | 3 | 659 | g2984157 | 4.00E−28 | ribosomal protein L13 |
| 345 | 2 | 134 | 95 | 496 | g14042747 | 4.00E−63 | unnamed protein product |
| 345 | 2 | 134 | 95 | 496 | g10440516 | 4.00E−63 | FLJ00106 protein |
| 345 | 2 | 134 | 95 | 496 | g10445215 | 7.00E−62 | PDZ-LIM protein mystique |
| 346 | 3 | 174 | 3 | 524 | g11527997 | 1.00E−112 | NOTCH2 protein |
| 346 | 3 | 174 | 3 | 524 | g11275978 | 1.00E−112 | NOTCH 2 |
| 346 | 3 | 174 | 3 | 524 | g287990 | 1.00E−112 | Motch B |
| 347 | 1 | 306 | 202 | 1119 | g14024759 | 4.00E−32 | aldehyde dehydrogenase |
| 347 | 1 | 306 | 202 | 1119 | g13162101 | 3.00E−28 | putative aldehyde dehydrogenase |
| 347 | 1 | 306 | 202 | 1119 | g14025513 | 1.00E−23 | succinate-semialdehyde dehydrogenase |
| 348 | 3 | 283 | 819 | 1667 | g10438978 | 1.00E−156 | unnamed protein product |
| 348 | 3 | 283 | 819 | 1667 | g13358630 | 1.00E−155 | hypothetical protein |
| 348 | 3 | 283 | 819 | 1667 | g9280094 | 1.00E−36 | unnamed protein product |
| 349 | 3 | 188 | 123 | 686 | g4126809 | 5.00E−88 | glyoxalase I |
| 349 | 3 | 188 | 123 | 686 | g1808684 | 3.00E−84 | hypothetical protein |
| 349 | 3 | 188 | 123 | 686 | g2213425 | 8.00E−81 | hypothetical protein |
| 350 | 1 | 176 | 1 | 528 | g57469 | 2.00E−98 | vasopressin |
| 350 | 1 | 176 | 1 | 528 | g4469315 | 2.00E−98 | vasopressin precursor |
| 350 | 1 | 176 | 1 | 528 | g207674 | 3.00E−96 | vasopressin/neurophysin precursor |
| 351 | 1 | 52 | 253 | 408 | g3790135 | 3.00E−18 | dJ191N21.3 (proteasome subunit HC5) |
| 351 | 1 | 52 | 253 | 408 | g220026 | 3.00E−18 | proteasome subunit C5 |
| 351 | 1 | 52 | 253 | 408 | g1698584 | 3.00E−18 | proteasome beta-subunit C5 |
| 352 | 3 | 190 | 3 | 572 | g205662 | 5.00E−86 | nucleoside diphosphate kinase |
| 352 | 3 | 190 | 3 | 572 | g206580 | 4.00E−85 | RBL-NDP kinase 18 kDa subunit (p18) |
| 352 | 3 | 190 | 3 | 572 | g53354 | 5.00E−85 | nucleoside diphosphate kinase B |
| 353 | 3 | 135 | 546 | 950 | g9368839 | 1.00E−37 | hypothetical protein |
| 353 | 3 | 135 | 546 | 950 | g57690 | 9.00E−29 | ribosomal protein L23a |
| 353 | 3 | 135 | 546 | 950 | g404015 | 9.00E−29 | ribosomal protein L23a |
| 354 | 1 | 517 | 1 | 1551 | g13161184 | 0 | cytochrome P450 2S1 |
| 354 | 1 | 517 | 1 | 1551 | g14042396 | 0 | unnamed protein product |
| 354 | 1 | 517 | 1 | 1551 | g12836063 | 0 | putative |
| 355 | 2 | 135 | 203 | 607 | g7677318 | 5.00E−55 | aldehyde reductase |
| 355 | 2 | 135 | 203 | 607 | g12848322 | 5.00E−55 | putative |
| 355 | 2 | 135 | 203 | 607 | g12848318 | 5.00E−55 | putative |
| 356 | 3 | 77 | 42 | 272 | g3851089 | 1.00E−24 | guanine nucleotide binding protein gamma 5 |
| 356 | 3 | 77 | 42 | 272 | g3329380 | 1.00E−24 | G protein gamma 5 subunit |
| 356 | 3 | 77 | 42 | 272 | g204241 | 1.00E−24 | G protein gamma-5 subunit |
| 357 | 3 | 110 | 96 | 425 | g7340072 | 4.00E−15 | 40S ribosomal protein S15A |
| 357 | 3 | 110 | 96 | 425 | g495273 | 4.00E−15 | ribosomal protein S15a |
| 357 | 3 | 110 | 96 | 425 | g12859337 | 4.00E−15 | putative |
| 358 | 1 | 485 | 1 | 1455 | g10436300 | 0 | unnamed protein product |
| 358 | 1 | 485 | 1 | 1455 | g10433852 | 0 | unnamed protein product |
| 358 | 1 | 485 | 1 | 1455 | g9280029 | 0 | unnamed protein product |
| 360 | 1 | 143 | 1 | 429 | g63466 | 2.00E−56 | histone H2A |
| 360 | 1 | 143 | 1 | 429 | g6094631 | 2.00E−56 | histone H2A.F |
| 360 | 1 | 143 | 1 | 429 | g3420799 | 2.00E−56 | histone H2A.F/Z variant |
| 361 | 3 | 81 | 159 | 401 | g10437078 | 4.00E−34 | unnamed protein product |
| 361 | 3 | 81 | 159 | 401 | g12859774 | 600E−33 | putative |
| 361 | 3 | 81 | 159 | 401 | g12857408 | 6.00E−33 | putative |
| 362 | 3 | 543 | 834 | 2462 | g12849316 | 4.00E−87 | putative |
| 362 | 3 | 543 | 834 | 2462 | g6164628 | 3.00E−86 | SH3 and PX domain-containing protein SH3PX1 |
| 362 | 3 | 543 | 834 | 2462 | g5410249 | 3.00E−86 | SDP1 protein |
| 363 | 1 | 185 | 1 | 555 | g12836716 | 1.00E−103 | putative |
| 363 | 1 | 185 | 1 | 555 | g12834312 | 1.00E−103 | putative |
| 363 | 1 | 185 | 1 | 555 | g12832330 | 1.00E−103 | putative |
| 364 | 3 | 156 | 177 | 644 | g14456629 | 1.00E−31 | dJ54B20.2 (novel KRAB box containing |
| | | | | | | | C2H2 type zinc finger protein) |
| 364 | 3 | 156 | 177 | 644 | g4589588 | 9.00E−27 | KIAA0972 protein |
| 364 | 3 | 156 | 177 | 644 | g3970712 | 3.00E−23 | zinc finger protein 10 |
| 365 | 1 | 183 | 235 | 783 | g12248382 | 7.00E−31 | SEMB |
| 365 | 1 | 183 | 235 | 783 | g854326 | 7.00E−29 | semaphorin B |
| 365 | 1 | 183 | 235 | 783 | g1110599 | 1.00E−14 | semaphorin homolog = M-Sema F |
| | | | | | | | (mice, neonatal brain, Peptide, 834 aa) |
| 366 | 1 | 416 | 1 | 1248 | g13543419 | 1.00E−161 | Similar to zinc finger protein 304 |
| 366 | 1 | 416 | 1 | 1248 | g7020745 | 5.00E−53 | unnamed protein product |
| 366 | 1 | 416 | 1 | 1248 | g12652759 | 5.00E−53 | hypothetical protein FLJ20557 |
| 367 | 2 | 258 | 455 | 1228 | g1174187 | 2.00E−94 | purine nucleotide binding protein |
| 367 | 2 | 258 | 455 | 1228 | g193444 | 8.00E−88 | guanylate binding protein |
| 367 | 2 | 258 | 455 | 1228 | g829177 | 1.00E−68 | guanylate binding protein isoform II |
| 368 | 3 | 68 | 270 | 473 | g14586963 | 7.00E−10 | (AF362574) M75 |
| 368 | 3 | 68 | 270 | 473 | g57115 | 7.00E−10 | ribosomal protein L31 (AA 1-125) |
| 368 | 3 | 68 | 270 | 473 | g36130 | 7.00E−10 | ribosomal protein L31 (AA 1-125) |
| 371 | 1 | 122 | 568 | 933 | g10435150 | 2.00E−39 | unnamed protein product |
| 371 | 1 | 122 | 568 | 933 | g4982195 | 2.00E−16 | lepA protein |
| 371 | 1 | 122 | 568 | 933 | g2984041 | 6.00E−16 | G-protein LepA |
| 372 | 3 | 111 | 3 | 335 | g13752754 | 3.00E−15 | zinc finger 1111 |
| 372 | 3 | 111 | 3 | 335 | g7023216 | 2.00E−14 | unnamed protein product |
| 372 | 3 | 111 | 3 | 335 | g14348588 | 2.00E−14 | KRAB zinc finger protein |
| 373 | 3 | 1281 | 36 | 3878 | g157409 | 2.00E−95 | fat protein |
| 373 | 3 | 1281 | 36 | 3878 | g7295732 | 3.00E−94 | ft gene product |
| 373 | 3 | 1281 | 36 | 3878 | g10727403 | 2.00E−93 | ds gene product |
| 374 | 3 | 164 | 3 | 494 | g9759463 | 2.00E−60 | 40S ribosomal protein S19 |
| 374 | 3 | 164 | 3 | 494 | g6513924 | 4.00E−60 | putative 40S ribosomal protein S19 |
| 374 | 3 | 164 | 3 | 494 | g13878029 | 4.00E−60 | putative 40S ribosomal protein S19 |
| 375 | 3 | 365 | 750 | 1844 | g55628 | 0 | reading frame (preproalbumin) |
| 375 | 3 | 365 | 750 | 1844 | g3647327 | 0 | serum albumin |
| 375 | 3 | 365 | 750 | 1844 | g12845183 | 0 | putative |
| 376 | 1 | 96 | 184 | 471 | g12853416 | 5.00E−25 | putative |
| 376 | 1 | 96 | 184 | 471 | g13529497 | 8.00E−23 | Unknown (protein for MGC: 6652) |
| 376 | 1 | 96 | 184 | 471 | g4589588 | 5.00E−22 | KIAA0972 protein |
| 377 | 2 | 106 | 125 | 442 | g14042293 | 3.00E−37 | unnamed protein product |
| 377 | 2 | 106 | 125 | 442 | g12052983 | 3.00E−37 | hypothetical protein |
| 377 | 2 | 106 | 125 | 442 | g14043841 | 7.00E−34 | Unknown (protein for MGC: 14429) |
| 378 | 2 | 119 | 2 | 358 | g57710 | 2.00E−31 | ribosomal phosphoprotein P1 (AA 1-114) |
| 378 | 2 | 119 | 2 | 358 | g190234 | 2.00E−31 | acidic ribosomal phosphoprotein (P1) |
| 378 | 2 | 119 | 2 | 358 | g14043204 | 2.00E−31 | ribosomal protein, large, P1 |
| 380 | 2 | 282 | 2 | 847 | g10440398 | 8.00E−93 | FLJ00032 protein |
| 380 | 2 | 282 | 2 | 847 | g10047297 | 8.00E−93 | KIAA1611 protein |
| 380 | 2 | 282 | 2 | 847 | g10436789 | 6.00E−92 | unnamed protein product |
| 381 | 2 | 47 | 287 | 427 | g7290056 | 2.00E−10 | EG: 118B3.2 gene product (alt 2) |
| 381 | 2 | 47 | 287 | 427 | g5901822 | 2.00E−10 | EG: 118B3.2 |
| 381 | 2 | 47 | 287 | 427 | g3645991 | 2.00E−10 | /prediction = (method: ““genefinder””, |
| | | | | | | | version: ““084””, |
| | | | | | | | score: ““128.32””)˜/ |
| | | | | | | | prediction = (method: ““genscan””, |
| | | | | | | | version: ““1.0””)˜/ |
| | | | | | | | match = (desc: ““KIAA0376 PROTEIN |
| | | | | | | | (FRAGMENT)””, species: ““HOMO |
| | | | | | | | SAPIENS (HUMAN)””, |
| | | | | | | | ranges: (query:15779 . . . 16030, |
| | | | | | | | target: SPTREMBL::O15081:314 . . . 231, |
| | | | | | | | score: ““229.00””), |
| | | | | | | | (query:14786 . . . 15736, |
| | | | | | | | target: SPTREMBL::O15081: 650 . . . 334, |
| | | | | | | | score: ““319.00””), |
| | | | | | | | (query: 13868 . . . 13960, |
| | | | | | | | target: SPTREMBL::O15081: 884 . . . 854, |
| | | | | | | | score:““156.00””)), |
| | | | | | | | method: ““blastx””, |
| | | | | | | | version: ““1.4.9””)˜/ |
| | | | | | | | match = (desc: ““SPECTRIN |
| | | | | | | | BETA CHAIN, ERYTHROCYTE””, |
| | | | | | | | species: ““HOMO SAPIENS (HUMAN) |
| | | | | | | | ””, ranges: (query: 13748 . . . 13954, |
| | | | | | | | target: SWISS-PROT::P11277: 242 . . . 174, |
| | | | | | | | score: ““201.00””)), |
| | | | | | | | method:““blastx””, |
| | | | | | | | version: ““1.4.9””)˜/ |
| | | | | | | | match = (desc: ““ |
| | | | | | | | GH04661.5prime GH Drosophila |
| | | | | | | | melanogaster head pOT2 Drosophila |
| | | | | | | | melanogaster cDNA clone GH046615 |
| | | | | | | | prime, mRNA sequence””, |
| | | | | | | | species: ““Drosophila melanogaster (fruit fly)””, |
| | | | | | | | ranges: (query: 22139 . . . 22499, |
| | | | | | | | target: EMBL::AI064293: 361 . . . 1, |
| | | | | | | | score: ““1796.00””)), |
| | | | | | | | method: ““blastn””, |
| | | | | | | | version: ““1.4.9””)˜/ |
| | | | | | | | match = (desc: ““GH05563.5prime |
| | | | | | | | GH Drosophila |
| | | | | | | | melanogaster head pOT2 Drosophila |
| | | | | | | | melanogaster cDNA clone GH05563 |
| 383 | 1 | 280 | 16 | 855 | g14456629 | 9.00E−90 | dJ54B20.2 |
| | | | | | | | (novel KRAB box containing |
| | | | | | | | C2H2 type zinc finger protein) |
| 383 | 1 | 280 | 16 | 855 | g3378094 | 3.00E−72 | KRAB domain zinc finger protein |
| 383 | 1 | 280 | 16 | 855 | g1020145 | 1.00E−71 | DNA binding protein |
| 384 | 3 | 264 | 3 | 794 | g6807587 | 1.00E−124 | hypothetical protein |
| 384 | 3 | 264 | 3 | 794 | g5931821 | 1.00E−124 | dJ228H13.3 (zinc finger protein) |
| 384 | 3 | 264 | 3 | 794 | g488555 | 1.00E−96 | zinc finger protein ZNF135 |
| 385 | 3 | 561 | 135 | 1817 | g10954044 | 0 | KRAB zinc finger protein ZFQR |
| 385 | 3 | 561 | 135 | 1817 | g10442700 | 0 | zinc-finger protein ZBRK1 |
| 385 | 3 | 561 | 135 | 1817 | g10435411 | 0 | unnamed protein product |
| 386 | 2 | 288 | 206 | 1069 | g10439850 | 4.00E−70 | unnamed protein product |
| 386 | 2 | 288 | 206 | 1069 | g4894364 | 6.00E−64 | zinc finger protein 3 |
| 386 | 2 | 288 | 206 | 1069 | g12655165 | 6.00E−64 | zinc finger protein 256 |
| 387 | 3 | 476 | 171 | 1598 | g14042538 | 0 | unnamed protein product |
| 387 | 3 | 476 | 171 | 1598 | g10438630 | 0 | unnamed protein product |
| 387 | 3 | 476 | 171 | 1598 | g13096919 | 0 | Similar to zinc finger protein 135 (clone pHZ-17) |
| 389 | 3 | 60 | 78 | 257 | g5262560 | 5.00E−07 | hypothetical protein |
| 389 | 3 | 60 | 78 | 257 | g10434856 | 5.00E−07 | unnamed protein product |
| 389 | 3 | 60 | 78 | 257 | g13623354 | 6.00E−07 | Similar to zinc finger protein 136 (clone pHZ-20) |
| 390 | 2 | 355 | 254 | 1318 | g10047183 | 1.00E−94 | KIAA1559 protein |
| 390 | 2 | 355 | 254 | 1318 | g13676461 | 6.00E−81 | hypothetical protein |
| 390 | 2 | 355 | 254 | 1318 | g4589566 | 1.00E−80 | KIAA0961 protein |
| 391 | 1 | 101 | 112 | 414 | g13937999 | 3.00E−48 | Similar to DNA-binding protein |
| 391 | 1 | 101 | 112 | 414 | g3329372 | 1.00E−34 | DNA-binding protein |
| 391 | 1 | 101 | 112 | 414 | g12052732 | 3.00E−34 | hypothetical protein |
| 392 | 3 | 326 | 3 | 980 | g14017921 | 0 | KIAA1852 protein |
| 392 | 3 | 326 | 3 | 980 | g8050899 | 1.00E−133 | ZNF180 |
| 392 | 3 | 326 | 3 | 980 | g6409345 | 1.00E−133 | zinc finger protein ZNF180 |
| 393 | 3 | 263 | 792 | 1580 | g5441615 | 1.00E−96 | zinc finger protein |
| 393 | 3 | 263 | 792 | 1580 | g498721 | 1.00E−94 | zinc finger protein |
| 393 | 3 | 263 | 792 | 1580 | g8099348 | 2.00E−93 | zinc finger protein |
| 394 | 3 | 121 | 372 | 734 | g10439850 | 3.00E−18 | unnamed protein product |
| 394 | 3 | 121 | 372 | 734 | g14249844 | 7.00E−18 | Similar to hypothetical protein FLJ23233 |
| 394 | 3 | 121 | 372 | 734 | g2689441 | 4.00E−13 | F18547_1 |
| 395 | 2 | 298 | 2 | 895 | g10437560 | 0 | unnamed protein product |
| 395 | 2 | 298 | 2 | 895 | g10047305 | 0 | KIAA1615 protein |
| 395 | 2 | 298 | 2 | 895 | g498721 | 4.00E−86 | zinc finger protein |
| 396 | 1 | 287 | 220 | 1080 | g14042550 | 3.00E−94 | unnamed protein product |
| 396 | 1 | 287 | 220 | 1080 | g13937909 | 3.00E−94 | Similar to KIAA0961 protein |
| 396 | 1 | 287 | 220 | 1080 | g10047183 | 5.00E−78 | KIAA1559 protein |
| 397 | 1 | 281 | 277 | 1119 | g3540177 | 1.00E−123 | F23269_2 |
| 397 | 1 | 281 | 277 | 1119 | g5080758 | 1.00E−120 | BC331191_1 |
| 397 | 1 | 281 | 277 | 1119 | g12855931 | 5.00E−73 | putative |
| 398 | 1 | 263 | 1 | 789 | g10434650 | 4.00E−88 | unnamed protein product |
| 398 | 1 | 263 | 1 | 789 | g13623217 | 2.00E−40 | Similar to hypothetical protein FLJ12895 |
| 398 | 1 | 263 | 1 | 789 | g7020855 | 6.00E−27 | unnamed protein product |
| 399 | 2 | 646 | 200 | 2137 | g14042373 | 0 | unnamed protein product |
| 399 | 2 | 646 | 200 | 2137 | g498152 | 0 | ha0946 protein is Kruppel-related. |
| 399 | 2 | 646 | 200 | 2137 | g7243633 | 0 | RB-associated KRAB repressor |
| 400 | 2 | 199 | 140 | 736 | g204123 | 5.00E−89 | ferritin light chain |
| 400 | 2 | 199 | 140 | 736 | g204133 | 6.00E−89 | ferritin light chain |
| 400 | 2 | 199 | 140 | 736 | g193275 | 6.00E−89 | ferritin light chain |
| 401 | 1 | 210 | 25 | 654 | g9651704 | 4.00E−79 | carboxypeptidase B precursor |
| 401 | 1 | 210 | 25 | 654 | g6013463 | 8.00E−61 | carboxypeptidase homolog |
| 401 | 1 | 210 | 25 | 654 | g203295 | 8.00E−61 | carboxypeptidase B |
| 402 | 2 | 390 | 2 | 1171 | g13365901 | 0 | hypothetical protein |
| 402 | 2 | 390 | 2 | 1171 | g2104689 | 1.00E−126 | alpha glucosidase II, alpha subunit |
| 402 | 2 | 390 | 2 | 1171 | g1890664 | 1.00E−126 | glucosidase II |
| 403 | 3 | 43 | 348 | 476 | g12052884 | 3.00E−06 | hypothetical protein |
| 403 | 3 | 43 | 348 | 476 | g7023332 | 4.00E−06 | unnamed protein product |
| 403 | 3 | 43 | 348 | 476 | g183002 | 6.00E−05 | guanylate binding protein isoform I |
| 404 | 1 | 406 | 1 | 1218 | g12053281 | 0 | hypothetical protein |
| 404 | 1 | 406 | 1 | 1218 | g12836135 | 1.00E−167 | putative |
| 404 | 1 | 406 | 1 | 1218 | g3043598 | 2.00E−64 | KIAA0537 protein |
| 405 | 1 | 90 | 1 | 270 | g7981261 | 4.00E−33 | dJ50O24.4 (novel protein with DHHC |
| | | | | | | | zinc finger domain) |
| 405 | 1 | 90 | 1 | 270 | g14035816 | 4.00E−33 | unnamed protein product |
| 405 | 1 | 90 | 1 | 270 | g12224992 | 4.00E−33 | hypothetical protein |
| 407 | 2 | 192 | 44 | 619 | g7297667 | 1.00E−30 | CG5022 gene product |
| 407 | 2 | 192 | 44 | 619 | g2224617 | 4.00E−30 | KIAA0338 |
| 407 | 2 | 192 | 44 | 619 | g13277297 | 4.00E−30 | bA234K24.1.2 (Erythrocyte membrane |
| | | | | | | | protein band 4.1-like 1 protein |
| | | | | | | | (KIAA0338) Isoform 2) |
| 408 | 1 | 183 | 28 | 576 | g12314083 | 9.00E−94 | dJ1007G16.5 (novel high-mobility |
| | | | | | | | group (nonhistone chromosomal) |
| | | | | | | | protein 2 (HMG2) like protein) |
| 408 | 1 | 183 | 28 | 576 | g12838247 | 4.00E−67 | putative |
| 408 | 1 | 183 | 28 | 576 | g1304193 | 9.00E−35 | HMG2 |
| 409 | 3 | 163 | 3 | 491 | g12697320 | 8.00E−51 | Pbx4 protein |
| 409 | 3 | 163 | 3 | 491 | g7160800 | 3.00E−50 | Pbx4/Lazarus homeodomain protein |
| 409 | 3 | 163 | 3 | 491 | g5679283 | 3.00E−50 | Pbx4 homeodomain protein |
| 410 | 3 | 186 | 198 | 755 | g12840673 | 2.00E−48 | putative |
| 410 | 3 | 186 | 198 | 755 | g607003 | 4.00E−18 | beta transducin-like protein |
| 410 | 3 | 186 | 198 | 755 | g3878300 | 1.00E−14 | predicted using Genefinder˜Similarity |
| | | | | | | | to C.elegans Guanine nucleotide |
| | | | | | | | binding protein (WP: C14B1.4), |
| | | | | | | | contains similarity to Pfam domain: PF00400 |
| | | | | | | | (WD domain, G-beta repeat), Score = 196.1, |
| | | | | | | | E-value = 1.8e−55, N = 7˜cDNA |
| | | | | | | | EST yk567g12.3 comes from this |
| | | | | | | | gene˜cDNA EST yk567g12.5 comes from |
| | | | | | | | this gene |
| 411 | 1 | 134 | 286 | 687 | g11342541 | 2.00E−67 | putative white family ATP-binding |
| | | | | | | | cassette transporter |
| 411 | 1 | 134 | 286 | 687 | g9665220 | 3.00E−50 | ATP-binding cassette transporter, |
| | | | | | | | sub-family G member 1 |
| 411 | 1 | 134 | 286 | 687 | g7768742 | 3.00E−50 | white protein homolog (ATP-binding |
| | | | | | | | cassette transporter 8) |
| 412 | 2 | 97 | 35 | 325 | g57175 | 1.00E−37 | S-100 protein |
| 412 | 2 | 97 | 35 | 325 | g206825 | 1.00E−37 | S100 protein |
| 412 | 2 | 97 | 35 | 325 | g404769 | 2.00E−37 | S100 beta protein |
| 413 | 1 | 181 | 1 | 543 | g13543071 | 9.00E−64 | RIKEN cDNA 1500031N16 gene |
| 413 | 1 | 181 | 1 | 543 | g12837801 | 9.00E−64 | putative |
| 413 | 1 | 181 | 1 | 543 | g12832973 | 5.00E−60 | putative |
| 414 | 3 | 258 | 3 | 776 | g12856949 | 4.00E−98 | putative |
| 414 | 3 | 258 | 3 | 776 | g12653785 | 3.00E−97 | Unknown (protein for IMAGE: 3349601) |
| 414 | 3 | 258 | 3 | 776 | g12845723 | 4.00E−94 | putative |
| 415 | 2 | 169 | 2 | 508 | g7259240 | 1.00E-79 | unnamed protein product |
| 415 | 2 | 169 | 2 | 508 | g12845457 | 1.00E−79 | putative |
| 415 | 2 | 169 | 2 | 508 | g12834293 | 1.00E−79 | putative |
| 416 | 1 | 177 | 310 | 840 | g9502403 | 5.00E−06 | Hypothetical zinc finger-like protein |
| 417 | 1 | 81 | 451 | 693 | g14042550 | 6.00E−27 | unnamed protein product |
| 417 | 1 | 81 | 451 | 693 | g13937909 | 6.00E−27 | Similar to KIAA0961 protein |
| 417 | 1 | 81 | 451 | 693 | g487787 | 3.00E−23 | zinc finger protein ZNF140 |
| 418 | 2 | 94 | 329 | 610 | g13752754 | 7.00E−18 | zinc finger 1111 |
| 418 | 2 | 94 | 329 | 610 | g14348588 | 1.00E−16 | KRAB zinc finger protein |
| 418 | 2 | 94 | 329 | 610 | g12654015 | 1.00E−16 | Similar to hypothetical protein FLJ10891 |
| 419 | 3 | 165 | 3 | 497 | g12856090 | 2.00E−51 | putative |
| 419 | 3 | 165 | 3 | 497 | g12854104 | 2.00E-51 | putative |
| 419 | 3 | 165 | 3 | 497 | g4678718 | 4.00E−43 | dJ2013.1 (brain mitochondrial carrier |
| | | | | | | | protein-1 (BMCP1)) |
| 420 | 3 | 352 | 111 | 1166 | g13444976 | 4.00E−36 | unnamed protein product |
| 420 | 3 | 352 | 111 | 1166 | g12309630 | 4.00E−36 | bA438B23.1 (neuronal leucine-rich |
| | | | | | | | repeat protein) |
| 420 | 3 | 352 | 111 | 1166 | g9651089 | 8.00E−34 | hypothetical protein |
| 421 | 2 | 113 | 2 | 340 | g14164613 | 3.00E−27 | sialic acid binding immunoglobulin-like |
| | | | | | | | lectin 10 |
| 421 | 2 | 113 | 2 | 340 | g13991167 | 6.00E−16 | sialic acid-binding immunoglobulin-like |
| | | | | | | | lectin-like long splice variant |
| 421 | 2 | 113 | 2 | 340 | g13991166 | 6.00E−16 | sialic acid-binding immunoglobulin-like |
| | | | | | | | lectin-like short splice variant |
| 422 | 2 | 100 | 101 | 400 | g12052732 | 1.00E−38 | hypothetical protein |
| 422 | 2 | 100 | 101 | 400 | g3329372 | 8.00E−37 | DNA-binding protein |
| 422 | 2 | 100 | 101 | 400 | g7959207 | 2.00E−34 | KIAA1473 protein |
| 423 | 1 | 117 | 1 | 351 | g7959207 | 800E−39 | KIAA1473 protein |
| 423 | 1 | 117 | 1 | 351 | g3342002 | 2.00E−36 | hematopoietic cell derived zinc finger protein |
| 423 | 1 | 117 | 1 | 351 | g186774 | 5.00E−35 | zinc finger protein |
| 424 | 2 | 250 | 2 | 751 | g14042850 | 2.00E−50 | unnamed protein product |
| 424 | 2 | 250 | 2 | 751 | g12052983 | 8.00E−37 | hypothetical protein |
| 424 | 2 | 250 | 2 | 751 | g14042293 | 1.00E−36 | unnamed protein product |
| 425 | 1 | 199 | 1 | 597 | g10437560 | 1.00E−105 | unnamed protein product |
| 425 | 1 | 199 | 1 | 597 | g10047305 | 1.00E−105 | KIAA1615 protein |
| 425 | 1 | 199 | 1 | 597 | g13436440 | 2.00E−77 | Unknown (protein for MGC: 4400) |
| 426 | 3 | 166 | 3 | 500 | g14017833 | 3.00E−86 | KIAA1808 protein |
| 426 | 3 | 166 | 3 | 500 | g4240175 | 6.00E−24 | KIAA0843 protein |
| 426 | 3 | 166 | 3 | 500 | g505094 | 5.00E−21 | similar to an actin bundling protein, dematn. |
| 427 | 1 | 157 | 1168 | 1638 | g14042421 | 4.00E−11 | unnamed protein product |
| 427 | 1 | 157 | 1168 | 1638 | g14017937 | 4.00E−11 | KIAA1860 protein |
| 427 | 1 | 157 | 1168 | 1638 | g13366084 | 4.00E−11 | MAP/microtubule affinity-regulating kinase like 1 |
| 429 | 3 | 200 | 3 | 602 | g9651099 | 1.00E−118 | hypothetical protein |
| 429 | 3 | 200 | 3 | 602 | g881564 | 1.00E−55 | ZNF157 |
| 429 | 3 | 200 | 3 | 602 | g453466 | 5.00E−55 | zinc finger protein |
| 430 | 2 | 173 | 2 | 520 | g11990770 | 2.00E−67 | bA534G20.1.1 (novel protein similar to |
| | | | | | | | Lysozyme C-1 (1,4-beta-N- |
| | | | | | | | acylmuramidase C, EC 3.2.1.17) (isoform 1)) |
| 430 | 2 | 173 | 2 | 520 | g11990771 | 1.00E−56 | bA534G20.1.2 (novel protein similar to |
| | | | | | | | Lysozyme C-1 (1,4-beta-N- |
| | | | | | | | acylmuramidase C, EC 3.2.1.17) (isoform 2)) |
| 430 | 2 | 173 | 2 | 520 | g12839824 | 3.00E−49 | putative |
| 431 | 1 | 181 | 76 | 618 | g13591714 | 4.00E−97 | (AF343664) immunoglobulin |
| | | | | | | | superfamily receptor translocation |
| | | | | | | | associated protein 2c |
| 431 | 1 | 181 | 76 | 618 | g13591712 | 4.00E−97 | (AF343663) immunoglobulin |
| | | | | | | | superfamily receptor translocation |
| | | | | | | | associated protein 2b |
| 431 | 1 | 181 | 76 | 618 | g13591710 | 4.00E−97 | (AF343662) immunoglobulin |
| | | | | | | | superfamily receptor translocation |
| | | | | | | | associated protein 2a |
| 433 | 1 | 157 | 22 | 492 | g7268562 | 7.00E−54 | ribosomal protein L32-like protein |
| 433 | 1 | 157 | 22 | 492 | g5816996 | 7.00E−54 | ribosomal protein L32-like protein |
| 433 | 1 | 157 | 22 | 492 | g13899057 | 2.00E−53 | ribosomal protein L32 |
| 434 | 1 | 182 | 157 | 702 | g12855141 | 5.00E−89 | putative |
| 434 | 1 | 182 | 157 | 702 | g12852338 | 5.00E−89 | putative |
| 434 | 1 | 182 | 157 | 702 | g12849745 | 5.00E−89 | putative |
| 435 | 2 | 134 | 2 | 403 | g2624328 | 2.00E−44 | OsGRP2 |
| 435 | 2 | 134 | 2 | 403 | g2366750 | 2.00E−34 | RNA binding protein |
| 435 | 2 | 134 | 2 | 403 | g7268089 | 8.00E−34 | glycine-rich RNA-binding protein AtGRP2-like |
| 436 | 2 | 148 | 731 | 1174 | g13397122 | 1.00E−58 | unnamed protein product |
| 436 | 2 | 148 | 731 | 1174 | g12654715 | 1.00E−58 | Similar to glucose regulated protein, 58 kDa |
| 436 | 2 | 148 | 731 | 1174 | g10728195 | 3.00E−15 | CG1837 gene product |
| 437 | 2 | 296 | 2 | 889 | g12597312 | 1.00E−149 | tRNA-guanine transglycosylase |
| 437 | 2 | 296 | 2 | 889 | g12597314 | 1.00E−137 | tRNA-guanine transglycosylase |
| 437 | 2 | 296 | 2 | 889 | g7415812 | 1.00E−135 | tRNA-guanine transglycosylase |
| 438 | 3 | 169 | 9 | 515 | g12406772 | 4.00E−52 | unnamed protein product |
| 438 | 3 | 169 | 9 | 515 | g12406688 | 4.00E−52 | unnamed protein product |
| 438 | 3 | 169 | 9 | 515 | g7299372 | 4.00E−29 | CG6567 gene product |
| 439 | 2 | 170 | 200 | 709 | g14133251 | 3.00E−93 | KIAA1479 protein |
| 439 | 2 | 170 | 200 | 709 | g2623162 | 7.00E−39 | semaphorin VIa |
| 439 | 2 | 170 | 200 | 709 | g11093909 | 7.00E−39 | axon guidance signal SEMA6A1 |
| 440 | 2 | 841 | 2 | 2524 | g11177164 | 0 | polydom protein |
| 440 | 2 | 841 | 2 | 2524 | g12060830 | 1.00E−155 | serologically defined breast |
| | | | | | | | cancer antigen NY-BR-38 |
| 440 | 2 | 841 | 2 | 2524 | g14198157 | 4.00E−83 | polydomain protein |
| 441 | 1 | 271 | 70 | 882 | g13898617 | 1.00E−142 | serine/threonine protein kinase SSTK |
| 441 | 1 | 271 | 70 | 882 | g13540326 | 1.00E−142 | serine/threonine kinase FKSG82 |
| 441 | 1 | 271 | 70 | 882 | g13898619 | 1.00E−140 | serine/threonine protein kinase SSTK |
| 442 | 2 | 311 | 743 | 1675 | g8979743 | 1.00E−160 | Band4.1-like5 protein |
| 442 | 2 | 311 | 743 | 1675 | g13278193 | 1.00E−153 | Similar to EHM2 gene |
| 442 | 2 | 311 | 743 | 1675 | g10434740 | 1.00E−140 | unnamed protein product |
| 443 | 2 | 529 | 416 | 2002 | g12834087 | 1.00E−155 | putative |
| 443 | 2 | 529 | 416 | 2002 | g2463628 | 6.00E−46 | putative monocarboxylate transporter |
| 443 | 2 | 529 | 416 | 2002 | g7328162 | 1.00E−40 | hypothetical protein |
| 444 | 1 | 335 | 4 | 1008 | g13159480 | 1.00E−128 | Translation may initiate at the ATG |
| | | | | | | | codon at nucleotides 40-42 or the ATG |
| | | | | | | | at nucleotides 43-45 |
| 444 | 1 | 335 | 4 | 1008 | g9229906 | 5.00E−35 | fibrinogen-like protein |
| 444 | 1 | 335 | 4 | 1008 | g387156 | 8.00E−33 | fibrinogen-like protein |
| 445 | 1 | 329 | 196 | 1182 | g4582324 | 0 | dJ708F5.1 (PUTATIVE novel Collagen |
| | | | | | | | alpha 1 LIKE protein) |
| 445 | 1 | 329 | 196 | 1182 | g12052774 | 0 | hypothetical protein |
| 445 | 1 | 329 | 196 | 1182 | g2326442 | 1.00E−38 | collagen type XII alpha 1 chain |
| 446 | 2 | 395 | 110 | 1294 | g12642596 | 0 | nuclear receptor co-repressor/ |
| | | | | | | | HDAC3 complex subunit TBLR1 |
| 446 | 2 | 395 | 110 | 1294 | g10434648 | 0 | unnamed protein product |
| 446 | 2 | 395 | 110 | 1294 | g12006104 | 0 | IRA1 |
| 447 | 1 | 163 | 1 | 489 | g12858551 | 7.00E−67 | putative |
| 447 | 1 | 163 | 1 | 489 | g12805285 | 7.00E−67 | Similar to ribosomal protein S27a |
| 447 | 1 | 163 | 1 | 489 | g1050756 | 7.00E−67 | fusion protein: ubiquitin (bases 43_513); |
| | | | | | | | ribosomal protein S27a (bases |
| | | | | | | | 217_532) |
| 448 | 3 | 78 | 330 | 563 | g8699209 | 5.00E−06 | cyclophilin A |
| 448 | 3 | 78 | 330 | 563 | g50621 | 5.00E−06 | cyclophilin (AA 1-164) |
| 448 | 3 | 78 | 330 | 563 | g49496 | 5.00E−06 | cyclophilin (AA 1-164) |
| 449 | 1 | 314 | 277 | 1218 | g12844770 | 1.00E−130 | putative |
| 449 | 1 | 314 | 277 | 1218 | g12861366 | 1.00E−127 | putative |
| 449 | 1 | 314 | 277 | 1218 | g12857383 | 7.00E−50 | putative |
| 450 | 1 | 130 | 181 | 570 | g2843171 | 4.00E−06 | zinc finger protein |
| 450 | 1 | 130 | 181 | 570 | g5817149 | 5.00E−06 | hypothetical protein |
| 450 | 1 | 130 | 181 | 570 | g10434142 | 5.00E−06 | unnamed protein product |
| 451 | 1 | 176 | 697 | 1224 | g13383265 | 1.00E−63 | actin related protein |
| 451 | 1 | 176 | 697 | 1224 | g13938319 | 3.00E−62 | Unknown (protein for MGC: 15664) |
| 451 | 1 | 176 | 697 | 1224 | g12840619 | 2.00E−51 | putative |
| 452 | 3 | 671 | 3 | 2015 | g12698057 | 1.00E−168 | KIAA1756 protein |
| 452 | 3 | 671 | 3 | 2015 | g1177322 | 1.00E−124 | CPG2 protein |
| 452 | 3 | 671 | 3 | 2015 | g10728577 | 1.00E−82 | Msp-300 gene product |
| 453 | 3 | 293 | 3 | 881 | g4235148 | 8.00E−85 | BC41195_1 |
| 453 | 3 | 293 | 3 | 881 | g14165525 | 7.00E−71 | Similar to CG8500 gene product |
| 453 | 3 | 293 | 3 | 881 | g14388336 | 2.00E−70 | hypothetical protein |
| 455 | 1 | 253 | 478 | 1236 | g14424576 | 1.00E−118 | hypothetical protein FLJ21963 |
| 455 | 1 | 253 | 478 | 1236 | g10438188 | 1.00E−118 | unnamed protein product |
| 455 | 1 | 253 | 478 | 1236 | g14025162 | 3.00E−57 | acetyl-coa synthetase |
| 457 | 1 | 125 | 529 | 903 | g12859335 | 1.00E−26 | putative |
| 457 | 1 | 125 | 529 | 903 | g12858578 | 1.00E−26 | putative |
| 457 | 1 | 125 | 529 | 903 | g12843085 | 6.00E−21 | putative |
| 458 | 1 | 394 | 577 | 1758 | g14533376 | 1.00E−148 | (AX151207) unnamed protein product |
| 458 | 1 | 394 | 577 | 1758 | g11762100 | 1.00E−144 | myo-inositol 1-phosphate synthase |
| 458 | 1 | 394 | 577 | 1758 | g3108053 | 1.00E−144 | myo-inositol 1-phosphate synthase; INO1 |
| 459 | 3 | 149 | 1137 | 1583 | g12053149 | 2.00E−39 | hypothetical protein |
| 460 | 2 | 323 | 122 | 1090 | g7297059 | 3.00E−06 | CG9117 gene product |
| 461 | 3 | 200 | 387 | 986 | g13365897 | 1.00E−82 | hypothetical protein |
| 461 | 3 | 200 | 387 | 986 | g2636109 | 2.00E−24 | similar to metabolite transport |
| | | | | | | | protein |
| 461 | 3 | 200 | 387 | 986 | g1894771 | 2.00E−24 | product highly similar to |
| | | | | | | | metabolite transport proteins |
| 462 | 2 | 406 | 701 | 1918 | g12697482 | 1.00E−121 | dJ583P15.7.2 (novel zinc finger protein |
| | | | | | | | similar to rat RIN ZF) |
| 462 | 2 | 406 | 701 | 1918 | g12855580 | 1.00E−81 | putative |
| 462 | 2 | 406 | 701 | 1918 | g12847599 | 7.00E−78 | putative |
| 463 | 3 | 181 | 102 | 644 | g13879442 | 2.00E−78 | Similar to RIKEN cDNA 2310035M22 gene |
| 463 | 3 | 181 | 102 | 644 | g12848905 | 2.00E−78 | putative |
| 463 | 3 | 181 | 102 | 644 | g12855490 | 1.00E−73 | putative |
| 464 | 3 | 137 | 69 | 479 | g11231085 | 7.00E−29 | hypothetical protein |
| 464 | 3 | 137 | 69 | 479 | g10998425 | 6.00E−09 | NORPEG-like protein |
| 464 | 3 | 137 | 69 | 479 | g10937641 | 6.00E−09 | ankycorbin |
| 465 | 2 | 278 | 218 | 1051 | g12861800 | 1.00E−111 | putative |
| 465 | 2 | 278 | 218 | 1051 | g3878713 | 2.00E−38 | (Z46935) weak similarity with quinone |
| | | | | | | | oxidoreductase, contains similarity |
| | | | | | | | to Pfam domain: PF00107 |
| | | | | | | | (Zinc-binding dehydrogenases), |
| | | | | | | | Score = −80.6, E- |
| | | | | | | | value = 6.2e−06, N = 1˜cDNA |
| | | | | | | | EST yk164b4.5 comes from this |
| | | | | | | | gene˜cDNA EST |
| | | | | | | | yk164b4.3 comes from this gene˜ |
| | | | | | | | cDNA EST yk264f3.5 comes from this |
| 465 | 2 | 278 | 218 | 1051 | g9948219 | 4.00E−36 | conserved hypothetical protein |
| 467 | 1 | 142 | 118 | 543 | g4559318 | 4.00E−17 | BC273239_1 |
| 467 | 1 | 142 | 118 | 543 | g186774 | 9.00E−17 | zinc finger protein |
| 467 | 1 | 142 | 118 | 543 | g1017722 | 9.00E−17 | repressor transcriptional factor |
| 468 | 3 | 126 | 78 | 455 | g9963804 | 5.00E−47 | zinc finger protein ZNF286 |
| 468 | 3 | 126 | 78 | 455 | g14017965 | 5.00E−47 | KIAA1874 protein |
| 468 | 3 | 126 | 78 | 455 | g5640017 | 2.00E−46 | zinc finger protein ZFP113 |
| 469 | 3 | 246 | 228 | 965 | g13676461 | 1.00E−45 | hypothetical protein |
| 469 | 3 | 246 | 228 | 965 | g4589566 | 1.00E−45 | KIAA0961 protein |
| 469 | 3 | 246 | 228 | 965 | g487787 | 2.00E−37 | zinc finger protein ZNF140 |
| 470 | 1 | 107 | 40 | 360 | g14348591 | 2.00E−28 | KRAB zinc finger protein |
| 470 | 1 | 107 | 40 | 360 | g7959207 | 1.00E−27 | KIAA1473 protein |
| 470 | 1 | 107 | 40 | 360 | g186774 | 4.00E−27 | zinc finger protein |
| 471 | 3 | 357 | 3 | 1073 | g12052983 | 1.00E−167 | hypothetical protein |
| 471 | 3 | 357 | 3 | 1073 | g14042293 | 1.00E−135 | unnamed protein product |
| 471 | 3 | 357 | 3 | 1073 | g5262560 | 1.00E−113 | hypothetical protein |
| 472 | 3 | 90 | 3 | 272 | g13752754 | 2.00E−16 | zinc flnger 1111 |
| 472 | 3 | 90 | 3 | 272 | g14348588 | 9.00E−15 | KRAB zinc finger protein |
| 472 | 3 | 90 | 3 | 272 | g12654015 | 9.00E−15 | Similar to hypothetical protein FLJ10891 |
| 473 | 1 | 295 | 1 | 885 | g14495650 | 1.00E−75 | (BC009433) zinc finger protein 331; |
| | | | | | | | zinc finger protein 463 |
| 473 | 1 | 295 | 1 | 885 | g8575775 | 1.00E−75 | KRAB zinc finger protein |
| 473 | 1 | 295 | 1 | 885 | g13939858 | 1.00E−75 | RITA |
| 474 | 1 | 195 | 1 | 585 | g7020503 | 9.00E−68 | unnamed protein product |
| 474 | 1 | 195 | 1 | 585 | g8453103 | 1.00E−67 | zinc finger protein |
| 474 | 1 | 195 | 1 | 585 | g8099348 | 3.00E−67 | zinc finger protein |
| 475 | 2 | 232 | 257 | 952 | g14042293 | 4.00E−53 | unnamed protein product |
| 475 | 2 | 232 | 257 | 952 | g14042850 | 3.00E−47 | unnamed protein product |
| 475 | 2 | 232 | 257 | 952 | g12052983 | 6.00E−38 | hypothetical protein |
| 476 | 2 | 282 | 2 | 847 | g10440398 | 1.00E−93 | FLJ00032 protein |
| 476 | 2 | 282 | 2 | 847 | g10047297 | 1.00E−93 | KIAA1611 protein |
| 476 | 2 | 282 | 2 | 847 | g10436789 | 8.00E−93 | unnamed protein product |
| 477 | 3 | 149 | 3 | 449 | g12656631 | 7.00E−20 | Kruppel-like zinc finger protein GLIS2 |
| 477 | 3 | 149 | 3 | 449 | g13507039 | 2.00E−19 | Gli-Kruppel zinc-finger protein NKL |
| 477 | 3 | 149 | 3 | 449 | g13507037 | 2.00E−19 | Gli-Kruppel zinc-finger protein NKL |
| 478 | 3 | 283 | 3 | 851 | g13623633 | 1.00E−174 | Unknown (protein for MGC: 13105) |
| 478 | 3 | 283 | 3 | 851 | g488555 | 3.00E−98 | zinc finger protein ZNF135 |
| 478 | 3 | 283 | 3 | 851 | g10437767 | 9.00E−97 | unnamed protein product |
| 479 | 3 | 264 | 3 | 794 | g6807587 | 1.00E−124 | hypothetical protein |
| 479 | 3 | 264 | 3 | 794 | g5931821 | 1.00E−124 | dJ228H13.3 (zinc finger protein) |
| 479 | 3 | 264 | 3 | 794 | g488555 | 1.00E−96 | zinc finger protein ZNF135 |
| 480 | 1 | 201 | 430 | 1032 | g3540177 | 1.00E−117 | F23269_2 |
| 480 | 1 | 201 | 430 | 1032 | g5080758 | 6.00E−92 | BC331191_1 |
| 480 | 1 | 201 | 430 | 1032 | g12855931 | 1.00E−55 | putative |
| 481 | 1 | 283 | 880 | 1728 | g14017871 | 1.00E−143 | KIAA1827 protein |
| 481 | 1 | 283 | 880 | 1728 | g10436789 | 2.00E−97 | unnamed protein product |
| 481 | 1 | 283 | 880 | 1728 | g13752754 | 1.00E−95 | zinc finger 1111 |
| 482 | 2 | 101 | 131 | 433 | g14042373 | 1.00E−24 | unnamed protein product |
| 482 | 2 | 101 | 131 | 433 | g1389741 | 7.00E−24 | KRAB/zinc finger suppressor protein 1 |
| 482 | 2 | 101 | 131 | 433 | g9800824 | 2.00E−20 | bA179N14.1 (novel zinc finger protein) |
| 483 | 1 | 101 | 112 | 414 | g13937999 | 3.00E−48 | Similar to DNA-binding protein |
| 483 | 1 | 101 | 112 | 414 | g3329372 | 1.00E−34 | DNA-binding protein |
| 483 | 1 | 101 | 112 | 414 | g12052732 | 3.00E−34 | hypothetical protein |
| 484 | 2 | 297 | 215 | 1105 | g12862320 | 2.00E−51 | WDC146 |
| 486 | 3 | 108 | 462 | 785 | g12856025 | 1.00E−51 | putative |
| 486 | 3 | 108 | 462 | 785 | g12846941 | 4.00E−37 | putative |
| 486 | 3 | 108 | 462 | 785 | g13938537 | 2.00E−36 | Similar to RIKEN cDNA 4933430F16 gene |
| 487 | 2 | 122 | 122 | 487 | g4235144 | 4.00E−47 | BC39498_1 |
| 487 | 2 | 122 | 122 | 487 | g4235143 | 7.00E−34 | BC39498_3 |
| 487 | 2 | 122 | 122 | 487 | g8163824 | 4.00E−30 | krueppel-like zinc finger protein HZF2 |
| 488 | 1 | 182 | 148 | 693 | g12854977 | 1.00E−41 | putative |
| 488 | 1 | 182 | 148 | 693 | g7267246 | 1.00E−21 | putative adenosine deaminase |
| 488 | 1 | 182 | 148 | 693 | g7299138 | 1.00E−16 | CG11994 gene product |
| 489 | 2 | 114 | 293 | 634 | g5262523 | 3.00E−19 | hypothetical protein |
| 489 | 2 | 114 | 293 | 634 | g340170 | 3.00E−19 | orotidine 5′-monophosphate |
| | | | | | | | decarboxylase (EC4.1.1.23) |
| 489 | 2 | 114 | 293 | 634 | g340168 | 3.00E−19 | UMP synthase |
| 490 | 3 | 415 | 3 | 1247 | g2689442 | 0 | R28830_1 |
| 490 | 3 | 415 | 3 | 1247 | g1572600 | 1.00E−172 | Zik1 |
| 490 | 3 | 415 | 3 | 1247 | g12652759 | 1.00E−141 | hypothetical protein FLJ20557 |
| 491 | 3 | 43 | 348 | 476 | g12052884 | 3.00E−06 | hypothetical protein |
| 491 | 3 | 43 | 348 | 476 | g7023332 | 4.00E−06 | unnamed protein product |
| 491 | 3 | 43 | 348 | 476 | g183002 | 6.00E−05 | guanylate binding protein isoform I |
| 492 | 1 | 134 | 49 | 450 | g220637 | 4.00E−52 | zinc finger protein |
| 492 | 1 | 134 | 49 | 450 | g5730196 | 2.00E−51 | Kruppel-type zinc finger |
| 492 | 1 | 134 | 49 | 450 | g14456631 | 8.00E−51 | dJ54B20.4 (novel KRAB box containing |
| | | | | | | | C2H2 type zinc finger protein) |
| 493 | 3 | 239 | 3 | 719 | g14042330 | 2.00E−96 | unnamed protein product |
| 493 | 3 | 239 | 3 | 719 | g10954044 | 8.00E−92 | KRAB zinc finger protein ZFQR |
| 493 | 3 | 239 | 3 | 719 | g10442700 | 8.00E−92 | zinc-finger protein ZBRK1 |
| 495 | 2 | 185 | 134 | 688 | g13560888 | 2.00E−38 | EZFIT-related protein 1 |
| 495 | 2 | 185 | 134 | 688 | g7243243 | 2.00E−37 | KIAA1431 protein |
| 495 | 2 | 185 | 134 | 688 | g4567178 | 5.00E−35 | R31665_2 (AA 1-673) |
| 496 | 1 | 277 | 178 | 1008 | g4589588 | 4.00E−67 | KIAA0972 protein |
| 496 | 1 | 277 | 178 | 1008 | g498152 | 4.00E−51 | ha0946 protein is Kruppel-related. |
| 496 | 1 | 277 | 178 | 1008 | g6467204 | 3.00E−38 | gonadotropin inducible transcription |
| | | | | | | | repressor-3 |
| 497 | 3 | 241 | 3 | 725 | g14043841 | 3.00E−41 | Unknown (protein for MGC: 14429) |
| 497 | 3 | 241 | 3 | 725 | g14042293 | 6.00E−41 | unnamed protein product |
| 497 | 3 | 241 | 3 | 725 | g12052983 | 2.00E−40 | hypothetical protein |
| 498 | 2 | 251 | 1139 | 1891 | g12052983 | 1.00E−114 | hypothetical protein |
| 498 | 2 | 251 | 1139 | 1891 | g5262560 | 4.00E−68 | hypothetical protein |
| 498 | 2 | 251 | 1139 | 1891 | g10434856 | 1.00E−61 | unnamed protein product |
| 499 | 3 | 282 | 54 | 899 | g5080758 | 1.00E−103 | BC331191_1 |
| 499 | 3 | 282 | 54 | 899 | g3540177 | 1.00E−103 | F23269_2 |
| 499 | 3 | 282 | 54 | 899 | g12855931 | 1.00E−58 | putative |
| 500 | 1 | 442 | 28 | 1353 | g12652727 | 0 | Unknown (protein for IMAGE: 3352566) |
| 500 | 1 | 442 | 28 | 1353 | g488555 | 1.00E−97 | zinc finger protein ZNF135 |
| 500 | 1 | 442 | 28 | 1353 | g3378094 | 1.00E−95 | KRAB domain zinc finger protein |
| 501 | 3 | 292 | 3 | 878 | g488551 | 1.00E−97 | zinc finger protein ZNF132 |
| 501 | 3 | 292 | 3 | 878 | g13543419 | 2.00E−97 | Similar to zinc finger protein 304 |
| 501 | 3 | 292 | 3 | 878 | g1199604 | 3.00E−97 | zinc finger protein C2H2-25 |
| 502 | 1 | 166 | 1 | 498 | g12804419 | 2.00E−65 | Unknown (protein for MGC: 1136) |
| 502 | 1 | 166 | 1 | 498 | g12844790 | 5.00E−64 | putative |
| 502 | 1 | 166 | 1 | 498 | g13111895 | 3.00E−33 | Unknown (protein for MGC: 2627) |
| 504 | 1 | 406 | 1 | 1218 | g12053281 | 0 | hypothetical protein |
| 504 | 1 | 406 | 1 | 1218 | g12836135 | 1.00E−167 | putative |
| 504 | 1 | 406 | 1 | 1218 | g3043598 | 2.00E−64 | KIAA0537 protein |
| 505 | 1 | 332 | 1 | 996 | g10438696 | 1.00E−140 | unnamed protein product |
| 505 | 1 | 332 | 1 | 996 | g12847740 | 7.00E−11 | putative |
| 505 | 1 | 332 | 1 | 996 | g7022551 | 1.00E−09 | unnamed protein product |
| 506 | 2 | 183 | 2 | 550 | g4105619 | 1.00E−94 | SPAF |
| 506 | 2 | 183 | 2 | 550 | g12847023 | 1.00E−94 | putative |
| 506 | 2 | 183 | 2 | 550 | g7297973 | 5.00E−67 | CG5776 gene product |

TABLE 8

| | | | Parameter |
|---|---|---|---|
| Program | Description | Reference | Threshold |
| ABIFACTURA | A program that removes vector sequences and | Applied Biosystems, Foster City, CA. | |
| | masks ambiguous bases in nucleic acid sequences. | | |
| ABI/ | A Fast Data Finder useful in comparing and | Applied Biosystems, Foster City, CA; | Mismatch |
| PARACEL | annotating amino acid or nucleic acid sequences. | Paracel Inc., Pasadena, CA. | <50% |
| FDF | | | |
| ABI | A program that assembles nucleic acid sequences. | Applied Biosystems, Foster City, CA. | |
| AutoAssembler | | | |
| BLAST | A Basic Local Alignment Search Tool useful in | Altschul, S. F. et al. (1990) J. Mol. Biol. | ESTs: |
| | sequence similarity search for amino acid and | 215: 403-410; Altschul, S. F. et al. (1997) | Probability |
| | nucleic acid sequences. BLAST includes five | Nucleic Acids Res. 25: 3389-3402. | value = 1.0E−8 |
| | functions: blastp, blastn, blastx, tblastn, and tblastx. | | or less Full |
| | | | Length |
| | | | sequences: |
| | | | Probability |
| | | | value = |
| | | | 1.0E−10 or less |
| FASTA | A Pearson and Lipman algorithm that searches for | Pearson, W. R. and D. J. Lipman (1988) Proc. | ESTs: fasta E |
| | similarity between a query sequence and a group of | Natl. Acad Sci. USA 85: 2444-2448; Pearson, | value = |
| | sequences of the same type. FASTA comprises as | W. R. (1990) Methods Enzymol. 183: 63-98; | 1.06E−6 |
| | least five functions: fasta, tfasta, fastx, tfastx, and | and Smith, T. F. and M. S. Waterman (1981) | Assembled |
| | ssearch. | Adv. Appl. Math. 2: 482-489. | ESTs: fasta |
| | | | Identity = 95% |
| | | | or greater and |
| | | | Match length = |
| | | | 200 bases or |
| | | | greater; fastx E |
| | | | value = 1.0E−8 |
| | | | or less Full |
| | | | Length |
| | | | sequences: |
| | | | fastx score = |
| | | | 100 or greater |
| BLIMPS | A BLocks IMProved Searcher that matches a | Henikoff, S. and J. G. Henikoff (1991) Nucleic | Probability |
| | sequence against those in BLOCKS, PRINTS, | Acids Res. 19: 6565-6572; Henikoff, J. G. and | value = 1.0E−3 |
| | DOMO, PRODOM, and PFAM databases to search | S. Henikoff (1996) Methods Enzymol. | or less |
| | for gene families, sequence homology, and structural | 266: 88-105; and Attwood, T. K. et al. (1997) J. | |
| | fingerprint regions. | Chem. Inf. Comput. Sci. 37: 417-424. | |
| HMMER | An algorithm for searching a query sequence against | Krogh, A. et al. (1994) J. Mol. Biol. | PEAM hits: |
| | hidden Markov model (HMM)-based databases of | 235: 1501-1531; Sonnhammer, E. L. L. et al. | Probability |
| | protein family consensus sequences, such as PFAM. | (1988) Nucleic Acids Res. 26: 320-322; | value = 1.0E−3 |
| | | Durbin, R. et al. (1998) Our World View, in a | or less |
| | | Nutshell, Cambridge Univ. Press, pp. 1-350. | Signal peptide |
| | | | hits: Score = 0 |
| | | | or greater |
| ProfileScan | An algorithm that searches for structural and sequence | Gribskov, M. et al. (1988) CABIOS 4: 61-66; | Normalized |
| | motifs in protein sequences that match sequence patterns | Gribskov, M. et al. (1989) Methods Enzymol. | quality score ≧ |
| | defined in Prosite. | 183: 146-159; Bairoch, A. et al. (1997) | GCG-specified |
| | | Nucleic Acids Res. 25: 217-221. | “HIGH” value |
| | | | for that |
| | | | particular |
| | | | Prosite motif. |
| | | | Generally, |
| | | | score = |
| | | | 1.4-2.1. |
| Phred | A base-calling algorithm that examines automated | Ewing, B. et al. (1998) Genome Res. | |
| | sequencer traces with high sensitivity and probability. | 8: 175-185; Ewing, B. and P. Green | |
| | | (1998) Genome Res. 8: 186-194. | |
| Phrap | A Phils Revised Assembly Program including SWAT and | Smith, T. F. and M. S. Waterman (1981) Adv. | Score = 120 or |
| | CrossMatch, programs based on efficient implementation | Appl. Math. 2: 482-489; Smith, T.F. and M.S. | greater; |
| | of the Smith-Waterman algorithm, useful in searching | Waterman (1981) J. Mol. Biol. 147: 195-197; | Match length = |
| | sequence homology and assembling DNA sequences. | and Green, P., University of Washington, | 56 or greater |
| | | Seattle, WA. | |
| Consed | A graphical tool for viewing and editing Phrap assemblies. | Gordon, D. et al. (1998) Genome Res. 8: 195-202. | |
| SPScan | A weight matrix analysis program that scans protein | Nielson, H. et al. (1997) Protein Engineering | Score = 3.5 or |
| | sequences for the presence of secretory signal peptides. | 10: 1-6; Claverie, J.M. and S. Audic (1997) | greater |
| | | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos (1996) | |
| | determine orientation. | Protein Sci. 5: 363-371. | |
| TMHMMER | A program that uses a hidden Markov model (HMM) to | Sonnhammer, E. L. et al. (1998) Proc. Sixth Intl. | |
| | delineate transmembrane segments on protein sequences | Conf. on Intelligent Systems for Mol. Biol., | |
| | and determine orientation. | Glasgow et al., eds., The Am. Assoc. for Artificial | |
| | | Intelligence(AAAI) Press, Menlo Park, CA, and | |
| | | MIT Press, Cambridge, MA, pp. 175-182. | |
| Motifs | A program that searches amino acid sequences for patterns | Bairoch, A. et al. (1997) Nucleic Acids | |
| | that matched those defined in Prosite. | Res. 25: 217-221; Wisconsin Package | |
| | | Program Manual, version 9, page M51-59, Genetics | |
| | | Computer Group, Madison, WI. | |

What is claimed is:

1. An isolated polynucleotide selected from the group consisting of:
a) a polynucleotide comprising a polynucleotide sequence selected from the group consisting of SEQ ID NO:1-252,
b) a polynucleotide comprising a naturally occurring polynucleotide sequence at least 90% identical to a polynucleotide sequence selected from the group consisting of SEQ ID NO:1-252,
c) a polynucleotide complementary to the polynucleotide of a),
d) a polynucleotide complementary to the polynucleotide of b), and
e) an RNA equivalent of a)-d).

2. An isolated polynucleotide of claim 1, selected from the group consisting of SEQ ID NO:1-252.

3. An isolated polynucleotide comprising at least 30 contiguous nucleotides of a polynucleotide of claim 1.

4. An isolated polynucleotide comprising at least 60 contiguous nucleotides of a polynucleotide of claim 1.

5. A composition for the detection of expression of disease detection and treatment polynucleotides comprising at least one of the polynucleotides of claim 1 and a detectable label.

6. A method for detecting a target polynucleotide in a sample, said target polynucleotide having a sequence of a polynucleotide of claim 1, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

7. A method for detecting a target polynucleotide in a sample, said target polynucleotide having a sequence of a polynucleotide of claim 1, the method comprising:
a) hybridize [symbol] sample with a probe comprising at least [symbol] contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

8. A method of claim 7, wherein the probe comprises at least 30 contiguous nucleotides.

9. A method of claim 7, wherein the probe comprises at least 60 contiguous nucleotides.

10. A recombinant polynucleotide comprising a promoter sequence operably linked to a polynucleotide of claim 1.

11. A cell transformed with a recombinant polynucleotide of claim 10.

12. A transgenic organism comprising a recombinant polynucleotide of claim 10.

13. A method for producing a disease detection and treatment polypeptide encoded by a polynucleotide of claim 1, the method comprising:
a) culturing a cell under conditions suitable for expression of the disease detection and treatment polypeptide, wherein said cell is transformed with a recombinant polynucleotide comprising a promoter sequence operably linked to a polynucleotide of claim 1, and
b) recovering the disease detection and treatment polypeptide so expressed.

14. A method of claim 13, wherein the polypeptide has an amino acid sequence selected from the group consisting of SEQ ID NO:253-506.

15. An isolated disease detection and treatment polypeptide (MDDT) encoded by at least one of the polynucleotides of claim 2.

16. A method of screening for a test compound that specifically binds to the polypeptide of claim 15, the method comprising:
a) combining the polypeptide of claim 15 with at least one [symbol] compound under suitable conditions, and
b) detecting binding of the polypeptide of claim 15 to the test compound, thereby identifying a compound that specifically binds to the polypeptide of claim 15.

17. A microarray wherein at least one element of the microarray is a polynucleotide of claim 3.

18. A method for generating a transcript image of a sample which contains polynucleotides, the method comprising:
a) labeling the polynucleotides of the sample,
b contacting the elements of the microarray of claim 17 with the labeled polynucleotides of the sample under conditions suitable for the formation of a hybridization complex, and
c) quantifying the expression of the polynucleotides in the sample.

19. A method for screening a compound for effectiveness in altering expression of a target polynucleotide, wherein said target polynucleotide comprises a polynucleotide sequence of a polynucleotide of claim 1, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

20. A method for assessing toxicity of a test compound, said method comprising:
a) treating a biological sample containing nucleic acids with the test compound,
b) hybridizing the nucleic acids of the treated biological sample with a probe comprising at least 20 contiguous nucleotides of a polynucleotide of claim 1 under conditions whereby a specific hybridization complex is formed between said probe and a target polynucleotide in the biological sample, said target polynucleotide comprising a polynucleotide sequence of a polynucleotide of claim 1 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the untreated biological sample is indicative of toxicity of the test compound.

21. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, and wherein said target polynucleotide is a polynucleotide of claim 1.

22. An array of claim 21, wherein said first oligonucleotide or polynucleotide sequence is completely complementary to at least 30 contiguous nucleotides of said target polynucleotide.

23. An array of claim 21, wherein said first oligonucleotide or polynucleotide sequence is completely complementary to at least 60 contiguous nucleotides of said target polynucleotide

24. An array of claim 21, wherein said first oligonucleotide or polynucleotide sequence is completely complementary to said target polynucleotide.

25. An array of claim 21, which is a microarray.

26. An array of claim 21, further comprising said target polynucleotide hybridized to a nucleotide molecule comprising said first oligonucleotide or polynucleotide sequence.

27. An array of claim 21, wherein a linker joins at least one of said nucleotide molecules to said solid substrate.

28. An array of claim 21, wherein each distinct physical location on the substrate contains multiple nucleotide molecules, and the multiple nucleotide molecules at any single distinct physical location have the same sequence, and each distinct physical location on the substrate contains nucleotide molecules having a sequence which differs from the sequence of nucleotide molecules at another distinct physical location on the substrate.

29. An isolated polypeptide selected from the group consisting of:
a) a polypeptide comprising an amino acid sequence selected from the group consisting of SEQ ID NO:253-506,
b) a polypeptide comprising a naturally occurring amino acid sequence at least 90% identical [symbol] amino acid sequence selected from the g [symbol] consisting of SEQ ID NO:253-506,
c) a biologically active fragment of a polypeptide having an amino acid sequence selected from the group consisting of SEQ ID NO:253-506, and
d) an immunogenic fragment of a polypeptide having an amino acid sequence selected from the group consisting of SEQ ID NO:253-506.

30. An isolated polypeptide of claim 29, having a sequence selected from the group consisting of SEQ ID NO:253-506.

31. An isolated polynucleotide encoding a polypeptide of claim 29.

32. An isolated polynucleotide encoding a polypeptide of claim 30.

33. An isolated polynucleotide of claim 32, having a sequence selected from the group consisting of SEQ ID NO:1-252.

34. An isolated antibody which specifically binds to a disease detection and treatment polypeptide of claim 29.

35. A diagnostic test for a condition or disease associated with the expression of MDDT in a biological sample, the method comprising:
a) combining the biological sample with an antibody of claim 34, under conditions suitable for the antibody to bind the polypeptide and form an antibody:polypeptide complex, and
b) detecting the complex, wherein the presence of the complex correlates with the presence of the polypeptide in the biological sample.

36. The antibody of claim 34, wherein the antibody is:
a) a chimeric antibody,
b) a single chain antibody,
c) a Fab fragment,
d) a F(ab′)₂ fragment, or
e) a humanized antibody.

37. A composition comprising an antibody of claim 34 and an [symbol] ptable excipient.

38. A method of diagnosing a condition or disease associated with the expression of MDDT in a subject, comprising administering to said subject an effective amount of the composition of claim 37.

39. A composition of claim 37, wherein the antibody is labeled.

40. A method of diagnosing a condition or disease associated with the expression of MDDT in a subject, comprising administering to said subject an effective amount of the composition of claim 39.

41. A method of preparing a polyclonal antibody with the specificity of the antibody of claim 34, the method comprising:
a) immunizing an animal with a polypeptide having an amino acid sequence selected from the group consisting of SEQ ID NO:253-506, or an immunogenic fragment thereof, under conditions to elicit an antibody response,
b) isolating antibodies from said animal, and
c) screening the isolated antibodies with the polypeptide, thereby identifying a polyclonal antibody which binds specifically to a polypeptide having an amino acid sequence selected from the group consisting of SEQ ID NO:253-506.

42. An antibody produced by a method of claim 41.

43. A composition comprising the antibody of claim 42 and a suitable carrier.

44. A method of making a monoclonal antibody with the specificity of the antibody of claim 34, the method comprising:
a) immunizing an animal with a polypeptide having an amino acid sequence selected from the group consisting of SEQ ID NO:253-506, or an immunogenic fragment thereof, under conditions to elicit an antibody response,
b) isolating antibody producing cells from the animal,
c) fusing the antibody producing cells with immortalized cells to form monoclonal antibody-producing hybridoma cells,
d) culturing [symbol] hybridoma cells, and
e) isolating from the culture monoclonal antibody which binds specifically to a polypeptide having an amino acid sequence selected from the group consisting of SEQ ID NO:253-506.

45. A monoclonal antibody produced by a method of claim 44.

46. A composition comprising the antibody of claim 45 and a suitable carrier.

47. The antibody of claim 34, wherein the antibody is produced by screening a Fab expression library.

48. The antibody of claim 34, wherein the antibody is produced by screening a recombinant immunoglobulin library.

49. A method of detecting a polypeptide having an amino acid sequence selected from the group consisting of SEQ ID NO:253-506 in a sample, the method comprising:
a) incubating the antibody of claim 34 with a sample under conditions to allow specific binding of the antibody and the polypeptide, and
b) detecting specific binding, wherein specific binding indicates the presence of a polypeptide having an amino acid sequence selected from the group consisting of SEQ ID NO:253-506 in the sample.

50. A method of purifying a polypeptide having an amino acid sequence selected from the group consisting of SEQ ID NO:253-506 from a sample, the method comprising:
a) incubating the antibody of claim 34 with a sample under conditions to allow specific binding of the antibody and the polypeptide, and
b) separating the antibody from the sample and obtaining the purified polypeptide having an amino acid sequence selected from the group consisting of SEQ ID NO:253-506.

51. A composition comprising a polypeptide of claim 29 and a pharmaceutically acceptable excipient.

52. A composition of claim 51, wherein the polypeptide has an amino acid sequence of SEQ ID NO:253-506.

53. A method for treating a disease or condition associated with increased expression of functional MDDT, comprising administering to a patient in need of such treatment the composition of claim 51.

54. A method for screening a compound for effectiveness as an agonist of a polypeptide of claim 29, the method comprising:
a) exposing a sample comprising a polypeptide of claim 29 to a compound, and
b) detecting agonist activity in the sample.

55. A composition comprising an agonist compound identified by a method of claim 54 and a pharmaceutically acceptable excipient.

56. A method for treating a disease or condition associated with decreased expression of functional MDDT, comprising administering to a patient in need of such treatment a composition of claim 55.

57. A method for screening a compound for effectiveness as an antagonist of a polypeptide of claim 29, the method comprising:
a) exposing a sample comprising a polypeptide of claim 29 to a compound, and
b) detecting antagonist activity in the sample.

58. A composition comprising an antagonist compound identified by a method of claim 57 and a pharmaceutically acceptable excipient.

59. A method for treating a disease or condition associated with overexpression of functional MDDT, comprising administering to a patient in need of such treatment a composition of claim 58.

60. A method of screening for a compound that modulates the activity of the polypeptide of claim 29, said method comprising:
a) combining the polypeptide of claim 29 with at least one test compound under conditions permissive for the activity of the polypeptide of claim 29,
b) assessing the activity of the polypeptide of claim 29 in the presence of the test compound, and
c) comparing the activity of the polypeptide of claim 29 in the presence of the test compound with the activity of the polypeptide of claim 29 in the absence of the test compound wherein a change in the activity of the polypeptide of claim 29 in the presence of the test compound is indicative of a compound that modulates the activity of the polypeptide of claim 29.
